(19) United States
(12) Patent Application Publication
(10) Pub. No.: US 2018/0135108 A1
(43) Pub. Date: May 17, 2018

(54) METHOD FOR DETECTING BACTERIAL AND FUNGAL PATHOGENS

(71) Applicant: Board of Trustees of Michigan State University, East Lansing, MI (US)

(72) Inventor: Brett Eric Etchebarne, Okemos, MI (US)

(21) Appl. No.: 15/821,586

(22) Filed: Nov. 22, 2017

Related U.S. Application Data
(63) Continuation-in-part of application No. 14/600,696, filed on Jan. 20, 2015.
(60) Provisional application No. 61/929,175, filed on Jan. 20, 2014.

(51) Int. Cl. C12Q 1/689; C12Q 1/6895; C12Q 1/6806

(52) U.S. Cl. CPC C12Q 1/689; C12Q 1/6895; C12Q 2600/158; C12Q 2600/16; C12Q 1/6806

(57) ABSTRACT

This disclosure provides a method for detecting bacterial and fungal pathogens at clinically relevant concentrations in a clinical fluid sample using a visual color change test. The method includes the steps of: preparing the clinical fluid sample for heat lysing with water; heat lysing the clinical fluid sample to form a lysate that includes DNA and RNA forming a first mixture; mixing the lysate with a target primer set, EBT dye, a polymerase enzyme, and a chemical reaction buffer in a vial to form a reaction mixture; incubating the reaction mixture; amplifying the optional DNA and RNA and the target primer in the reaction mixture using LAMP; cooling the reaction mixture for a predetermined amount of time stopping the amplification; and identifying a color change in the reaction mixture that is indicative of the presence of bacterial or fungal pathogens.

### CROSS-REFERENCE TO RELATED APPLICATION

This application is a Continuation-in-Part of U.S. patent application Ser. No. 14/600,696 filed Jan. 20, 2015, which claims priority to and all the benefits of U.S. Provisional Patent Application No. 61/929,175, filed on Jan. 20, 2014, both of which are herein expressly incorporated by reference in their entireties.

### FIELD OF THE DISCLOSURE

The present disclosure relates to methods of pathogen detection and, more specifically, to particular methods of identification of pathogenic species and their antibiotic resistance.

### SEQUENCE LISTING

This disclosure, in accordance with 37 CFR § 1.52, incorporates by reference the sequence listing material contained within text file titled “063000-00124_ST25.txt”, created on Nov. 8, 2017 and totaling 11,000 bytes.

### BACKGROUND

Infection is one of the greatest problems faced by humanity and directly impacts care provided from every healthcare field. Care for the critically infected accounts for an enormous amount of health care resource expenditure. Every minute passing with untreated clinical infection can be reasonably expected to increase the total morbidity and mortality of the infected patient, while increasing the potential strength and pathogenicity of the infecting organism(s) as it survives, proliferates and spreads to other hosts while untreated. While the human body can overcome infection from most potential microbial invaders, those who are already sick or immunocompromised can fall victim to infection. Infections by dangerous pathogens are difficult to eliminate without proper antimicrobial therapy and spread readily among us. The length of time required for standard hospital testing of most microbial pathogens limits accurate and effective diagnosis and treatment of infection.

Battling infection is a major healthcare objective. Untreated infections can rapidly evolve toward the condition of sepsis in which the body begins to fail and resuscitation becomes critical and tenuous. Identification of infection with rapid antimicrobial treatment are primary goals of medical care, but precise identification of offending organisms is slow and broad spectrum empirical therapy is employed to cover most potential pathogens. Current methods for identification of bacterial pathogens in a clinical setting typically require days of time, or a four-to-eight hour growth phase followed by DNA extraction, purification and nucleic acid based amplification.

Sepsis, an overwhelming infection, causes millions of deaths worldwide yearly and is the most common cause of fatality in hospitalized patients. Sepsis and infectious disease management represents a large and growing challenge in the healthcare setting due to increased prevalence of antibiotic resistant microorganisms and is severely limited by any inability to rapidly diagnose the pathogen(s) responsible for a critical illness. Revolutionary acceleration in detection not only of specific pathogens but also their antibiotic resistance profiles from days to hours benefits individual patient health and also protects humans from growth and spread of highly pathogenic multi-drug-resistant organisms.

If untreated, septic patients may have hours to live. Thus, cultures of blood, and any area of the body suspected to be a primary site of infection, are drawn at the time that a septic patient is identified and broad-spectrum intravenous antibiotics are introduced to eliminate virtually all potential pathogens. Treatments are de-escalated in three to five days as laboratory results return, indicating the pathogenic strain(s) and antibiotic sensitivity profiles.

Delivering immediate, state-of-the-art molecular methods at the point-of-care (POC) for precise early determination of pathogens can greatly enhance the ability to diagnose and treat infection to combat the rise of multi-drug-resistant strains. Current sepsis management is severely limited by an inability to rapidly diagnose the pathogen(s) responsible for a critically ill patient's infection. When untreated, septic patients typically have hours to live. Thus, blood cultures are drawn from a patient at the time that sepsis is suspected and 3-4 broad-spectrum intravenous antibiotics are introduced to eliminate virtually all potential pathogens. Treatments are only de-escalated three to five days later as laboratory results return, indicating the pathogenic strain and its antibiotic sensitivity profile. Due to the extended length between diagnosis and de-escalation of treatment, there remains opportunity for improvement.

Rapid and accurate diagnoses, paired with aggressive and effective interventions, are important to stemming the disease process, maintaining economically feasible care and reducing long-term morbidity for infected patients. Sepsis is recognized as a major cause of morbidity and mortality in infected patients and is estimated to occur in 300 cases per 100,000 people in the United States and 18 million cases occur per year worldwide. Systematic approaches to early sepsis identification and intervention including timely broad-spectrum antibiotic coverage and adequate fluid volume resuscitation have yielded definite improvements in patient outcomes and health care resource utilization. It has been recognized that one of the limiting factors in treatment of sepsis in the hospital setting is the timeliness of pathogen identification and implementation of appropriate antimicrobial therapy. A recent review and meta-analysis of mortality of patients presenting to the emergency department and diagnosed with sepsis indicated that immediate antibiotic administration reduced patient mortality by up to 33%. The current “gold standard” of sepsis microbial identification is blood culture, which takes between two and five days for a definitive species identification. Antimicrobial agent susceptibility for the given organism is generally garnered within this same timeframe. However, in the period it takes for culture results to be obtained, broad-spectrum antibiotics may be provided to ensure organism eradication. This method of nonspecific antimicrobial coverage and lengthy identification period can be improved upon with rapid and accurate species identification as well as antibiotic resistance gene identification. Any chronological delay in successful treatment has consequences for the infected patient, strengthening of microbial populations against future drug administration, not to mention raising health care costs.

Treatment of systemic microbial infections is complicated by the inborn abilities of bacteria to develop resistance to antibiotic treatments by mutating the genes targeted by them or acquisition of resistance genes from other pathogens. Together, these problems can be further compounded by the development of multi-drug resistant “super bugs” that have increased pathogenicity and drive up patient morbidity, mortality and health care costs. Methicillin-resistant S. aureus (MRSA) and Vancomycin-resistant Enterococcus (VRE) are just two examples of commonly encountered sources of sepsis that pose difficulty in antibiotic eradication. By identifying the organism(s) responsible for infection one could potentially tailor an antibiotic regimen to facilitate immediate and optimal treatment to achieve early goal-directed therapy of patients on a level not previously achieved while stemming the antibiotic resistance epidemic that is spreading worldwide.

Traditional microbial gene sequencing has relied upon cultivated clonal cultures to produce the profile of diversity in a natural sample. These methods have determined that most pathogenic microbes found in sepsis patients can be relegated to a small number of infectious organisms. High-throughput genomic sequencing projects continuing over the past four decades have provided a thorough knowledge of the genomes of these pathogenic organisms. Using various methodologies, including traditional DNA PCR, RT-PCR, and mass spectroscopy, microbial species can be identified using non-culture based methods.

Previous attempts at achieving the goal of pathogen identification have been met with moderate success using a multiplex polymerase chain reaction approach, which is somewhat challenging given the technical expertise and limitations involved with this diagnostic approach. Recent publications aiming at rapid diagnostics show strong real-time PCR results after either culture and DNA extraction for specific detection of uropathogens, or from labor intensive Gas Chromatography—Mass Spectroscopy based diagnostics for respiratory pathogens. Accordingly, there remains opportunity for improvement.

### SUMMARY OF THE DISCLOSURE

This disclosure provides a method for detecting bacterial and fungal pathogens at clinically relevant concentrations in a clinical fluid sample using a visual color change test that includes the steps of: preparing the clinical fluid sample for heat lysing with water; heat lysing the clinical fluid sample to form a lysate that optionally includes bacterial and/or fungal DNA and RNA forming a first mixture; mixing the lysate with a target DNA primer and/or target RNA primer, Eriochrome Black T dye, a polymerase enzyme, and a chemical reaction buffer in a vial to form a reaction mixture; incubating the reaction mixture; amplifying the optional bacterial and/or fungal DNA and RNA and the target DNA primer and/or the target RNA primer in the reaction mixture using loop mediated isothermal amplification; cooling the reaction mixture for a predetermined amount of time thereby stopping the amplification; and identifying a color change in the reaction mixture which is indicative of the presence of bacterial or fungal pathogens in the clinical fluid sample at the clinically relevant concentrations.

In one embodiment, the disclosure provides a method for detecting bacterial and fungal pathogens at clinically relevant concentrations in a clinical fluid sample that includes the steps of: preparing the clinical fluid sample for heat lysing with water; heat lysing the clinical fluid sample to form a lysate that optionally includes bacterial and/or fungal DNA and RNA forming a first mixture; mixing the lysate with a target DNA primer and/or a target RNA primer, Syto 82 dye, a polymerase enzyme, and a chemical reaction buffer into a vial to form a reaction mixture; incubating the reaction mixture; amplifying the optional bacterial and/or fungal DNA and/or RNA and the target DNA primer and/or the RNA primer in the reaction mixture using loop mediated isothermal amplification; and analyzing the reaction mixture by thermocycler to detect the presence of bacterial or fungal pathogens in the clinical fluid sample at the clinically relevant concentrations.

In another embodiment, the disclosure provides a method for detecting bacterial and fungal pathogens at clinically relevant concentrations in clinical fluid samples using a visual color change test and a fluorescence test the method includes the steps of: preparing a first clinical fluid sample for heat lysing with water; heat lysing the first clinical fluid sample to form a first lysate that optionally includes bacterial and/or fungal DNA and/or RNA forming a first mixture; mixing the first lysate with a target DNA primer and/or a target RNA primer, Eriochrome Black T dye, a polymerase enzyme, and chemical reaction buffer in a vial to form a first reaction mixture; incubating the first reaction mixture; amplifying the optional bacterial and/or fungal DNA and/or RNA and the target DNA primer and/or the target RNA primer in the first reaction mixture using loop mediated isothermal amplification; cooling the first reaction mixture for a predetermined amount of time thereby stopping the amplification; identifying a color change in the first reaction mixture which is indicative of the presence of bacterial or fungal pathogens in the first clinical fluid sample at the clinically relevant concentrations; preparing a second clinical fluid sample for heat lysing with water; heat lysing the second clinical fluid sample to form a second lysate that optionally includes bacterial and/or fungal DNA and/or RNA forming a second mixture; mixing the second lysate with a second target DNA primer and/or a second target RNA primer, Syto 82 dye, a polymerase enzyme and a chemical reaction buffer in a vial to form a second reaction mixture; amplifying the optional bacterial and/or fungal DNA and/or RNA and the second target DNA primer and/or the second target RNA primer in the second reaction mixture using loop mediated isothermal amplification; and analyzing the second reaction mixture by thermocycler to detect the presence of bacterial or fungal pathogens in the second clinical fluid sample at the clinically relevant concentrations.

In various embodiments, the disclosure provides a method for improving a shelf stable target DNA/RNA primer for the detection of bacterial and fungal pathogens at clinically relevant concentrations in a clinical fluid sample. The method includes the steps of: adding a plurality of components to a vial, where the components are chosen from a predetermined amount of: trehalose, polymerase, a primer mix, glycerol, a surfactant, a serum albumin, dNTP, and magnesium sulfate; adding an indicator and a reaction buffer to the vial thereby forming a mixture; vortexing the vial including the mixture for a predetermined amount of time; exposing the mixture to room temperature ±5° C. for 24 hours; and sealing the vial for future use of the detection of bacterial and fungal pathogens at clinically relevant concentrations in a clinical fluid sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other advantages of the present disclosure will be readily appreciated, as the same becomes better understood by reference to the following detailed description when considered in connection with the accompanying drawings wherein:

FIG. 1 is a representative flowchart of one embodiment of the method;

FIG. 2 is a representative flowchart of an alternate embodiment of the method;

FIG. 3 is a table of the identified microorganism species and their particular genetic markers identified by embodiments of the method;

FIG. 4 is a table of pathogens with example LAMP primer sequences;

FIGS. 5(a) and 5(a)(continued) illustrate a table of various microorganisms and their antibiotic resistance levels;

FIG. 6 illustrates the naked-eye visual detection of color change from purple to blue using EBT-dye chelation for positive and negative results;

FIG. 7 is a flowchart of a method for improving the detection of bacterial and fungal pathogens at clinically relevant concentrations in a clinical fluid sample using a visual color change test;

FIG. 8 is a flowchart of a method for improving the detection of bacterial and fungal pathogens at clinically relevant concentrations in a clinical fluid sample;

FIG. 9 is a flowchart of a method for improving the detection of bacterial and fungal pathogens at clinically relevant concentrations in a clinical fluid sample using both a visual color change test and a fluorescence test;

FIG. 10 is a flowchart of a method for improving a shelf stable target DNA/RNA primer for the detection of bacterial and fungal pathogens at clinically relevant concentrations in a clinical fluid sample;

FIGS. 11(a) and 11(a)(continued) illustrate a table of target organisms by genus and species, target primers and sensitivity of assays in Colony Forming Units (CFU) per mL of a reaction derived from purified DNA, with in vitro dilution from urine and blood results, and LAMP primers;

FIG. 12(a) illustrates the clinical sample processing and analysis by illustrating the S. aureus detection by EBT-color change from purple to blue—Tubes 1 and 2 are negative and Tubes 3 and 4 are positive;

FIG. 12(b) illustrates the clinical sample analysis by illustrating the positive S. aureus clinical sample amplification by thermocycler (pink tracing) and the positive control S. aureus amplification (blue tracing);

FIGS. 13(a) and 13(a)(continued) illustrate a table of the LightCycler96 primer validation analysis per organism where the bold values represent time in minutes to the threshold for triplicate wells and triplicate experiments for spiked in purified DNA;

FIG. 14(a) illustrates the Eriochrome Black T (EBT) dye spectrophotometric analysis at wavelength 475 nm;

FIG. 14(b) illustrates the Eriochrome Black T (EBT) dye spectrophotometric analysis at wavelength 420 nm;

FIG. 14(c) illustrates the Eriochrome Black T (EBT) dye spectrophotometric analysis at wavelength 400 nm;

FIG. 14(d) illustrates the Eriochrome Black T (EBT) dye spectrophotometric analysis at wavelength 380 nm;

FIGS. 15(a)-(m) illustrate logs of genomic copies per reaction well detection in 5 minute intervals per organism for LightCycler96 primer validation;

FIG. 16(a) illustrates the minimum concentration for detection of the presence of Eriochrome Black T dye for ten-fold serial dilutions of purified genomic DNA from 500 pg/μL to 5 fg/μL with visual and spectrophotometric analysis at 5 minute intervals from 0-20 minutes;

FIG. 16(b) illustrates the minimum concentration for detection of the presence of Eriochrome Black T dye for ten-fold serial dilutions of purified genomic DNA from 500 pg/μL to 5 fg/μL with visual and spectrophotometric analysis at 5 minute intervals from 25-45 minutes;

FIGS. 17 and 17(continued) illustrates a table including data from the testing of the methods and clinical samples with hospital culture or equivalent testing and with a corresponding duplicate sample for LAMP testing and a key for the table;

FIG. 18 illustrates the matched clinical data between the hospital samples and the results of the present disclosure;

FIG. 19 illustrates a table of the plate stability from room temperature stable Syto 82 assay plate preparation;

FIG. 20 illustrates a table of the results generated from one embodiment of the method of this disclosure versus the hospital culture results by culture type;

FIG. 21 illustrates a table of a urinalysis comparison of one embodiment of this disclosure versus the hospital culture results by organism;

FIG. 22 illustrates a table of a plurality of mucocutaneous swab culture results versus the hospital culture results by organism generated by one embodiment this disclosure;

FIG. 23(a) illustrates a table of a urine culture summary of the clinical fluid samples tested in 2013 from January-December generated by one embodiment of this disclosure;

FIG. 23(b) illustrates a table of a blood and urine culture summary of the clinical fluid samples from Sparrow Hospital in 2013 from January-December generated by one embodiment of this disclosure;

FIGS. 24(a) and 24(a)(continued) illustrate a table of an antibiotic sensitivity summary from Sparrow and McLaren of Greater Lansing Hospital in 2015 generated by one embodiment of this disclosure.

### DETAILED DESCRIPTION OF THE DISCLOSURE

Acceleration in laboratory time-to-completion for accurate pathogen detection using a targeted approach can advance point-of-care infectious disease treatment from empiric to prescriptive medicine. The present disclosure demonstrates rapid genetic diagnostics methods utilizing loop-mediated isothermal amplification (LAMP) to test for 15 common infection pathogen targets, called the Infection Diagnosis Panel (In-Dx). Point-of-care (POC) diagnostics utilizing loop-mediated isothermal polymerase chain reaction amplification (LAMP) methods allow detection of 14 common pathogens as well as the methicillin resistance genetic marker mecA in less than one hour directly from human clinical samples. The Infection Diagnosis (In-Dx) panel is an in vitro diagnostic test. The method is utilized for detection of pathogens directly from clinical blood, urine, wound, sputum, stool and cerebrospinal fluid culture samples. The 15 common infection pathogen targets include Escherichia coli, Staphylococcus aureus, Enterococcus faecalis, Enterococcus faecium, Streptococcus pyogenes, Pseudomonas aeruginosa, Klebsiella pneumonia, Proteus mirabilis, Staphylococcus epidermidis, Streptococcus agalactiae, Candida albicans, Enterococcus casseliflavus, Enterococcus gallinarum, Clostridium difficile and the mecA methicillin resistance gene, which are targeted by the methods in the present disclosure. Included organisms account for greater than 68% of the bloodstream and 85% of the urinary tract infections in the Lansing, Mich. area.

In many embodiments, processing and analysis of samples is reliably completed within 60 minutes of sample collection for the target organisms. The results reveal sufficient concentrations of pathogen DNA, isolated from infected samples collected in parallel with hospital cultures, for extensive molecular diagnostic analysis. Pathogen targets on a panel can be substituted for any nucleic acid probed desired.

In another embodiment, a single-tube method for amplification of DNA at an isothermal temperature is known as loop-mediated isothermal amplification (LAMP). LAMP technology obviates the need for thermocycling for DNA/RNA amplification, which dramatically decreases the time to detection of low abundance DNA templates, typically lowering the threshold for identification from approximately three hours to less than 30 minutes.

LAMP reactions allow amplification of templates at a target temperature of between 60 to 67° C. utilizing a polymerase enzyme with high strand displacement and replicative activity amplifying two to three sets of DNA primers. LAMP generally employs at least four primers targeted precisely to targeting six distinct regions on the gene to maximize specificity. The high degree of amplification of a target DNA (or RNA, with transcription/replication enabled enzymes) achieved due to the high target specificity allows detectable signal to be produced via fluorescent or colorimetric dyes that intercalate or directly label DNA, allowing a correlation with the initial copy number and therefore quantitative measurement.

One of the advantages of LAMP is the potential for amplification with minimal sample preparation. Previous studies have shown LAMP amplification results after minimal processing from positive blood cultures and DNA purification for MRSA and for Chlamydia trachomatis from urine. In previous studies, minimal inhibition of the reaction was observed in blood, saliva, and urine samples spiked with bacterial pathogens. Thus, LAMP has high potential in the field of medical diagnostics. In terms of minimal sample processing and rapid turnover from sample collection to data. LAMP can also be used for the detection of the Human Immunodeficiency Virus, Mycobacterium tuberculosis, Plasmodium falciparum, Bacillus anthracis, Pseudomonas aeruginosa, Escherichia coli, Zika Virus, C. difficile, and Acinetobacter baumanii.

In various embodiment, the present disclosure rapidly tests the presence of common infectious pathogens from human clinical samples with minimal sample processing. The various embodiments of the present disclosure target the more common microbes present in Lansing, Mich. area hospital patients and compare two separate methodologies to determine detection limits with unique genes characteristic of each microbe.

In one embodiment, clinical pathogen concentrations may be estimated using both a fluorescent-based thermocycler unit (Roche LightCycler 96) and a parallel visual discrimination test utilizing EBT Dye-based reaction color change. In another embodiment, the presence of molecular targets in patient samples using one embodiment of this disclosure against hospital culture methods from prospectively-collected clinical patient samples across clinical culture types can be analyzed.

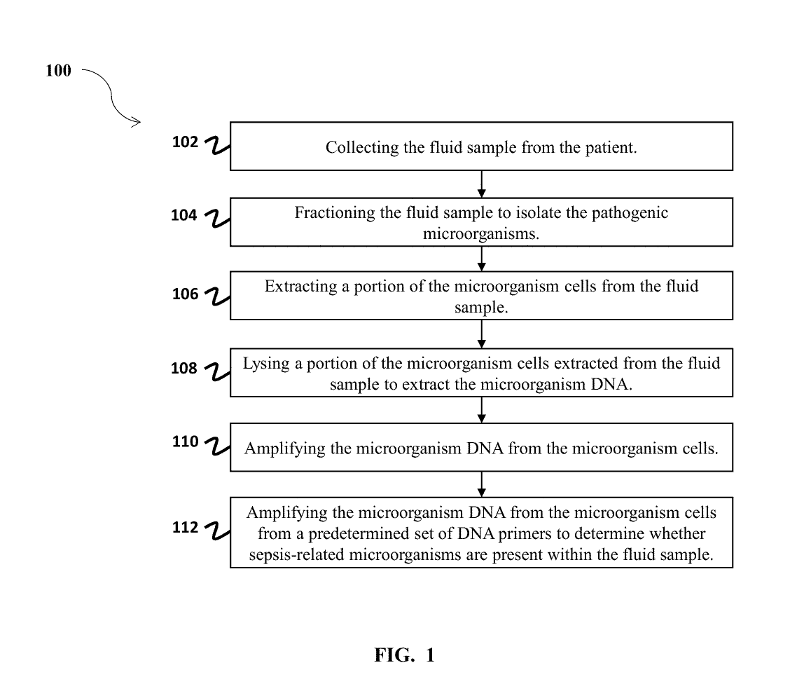

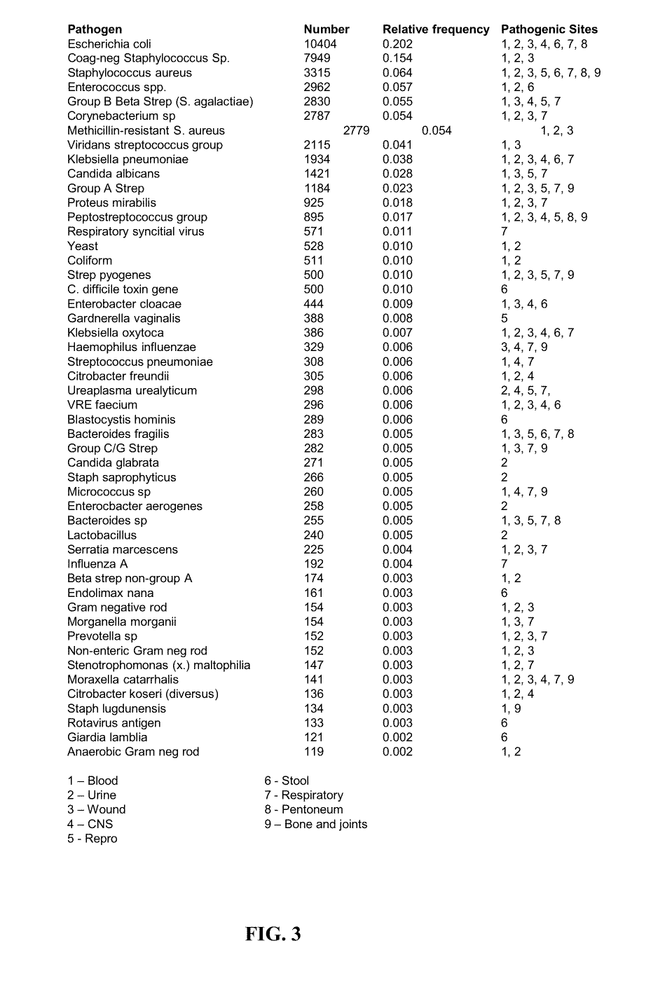

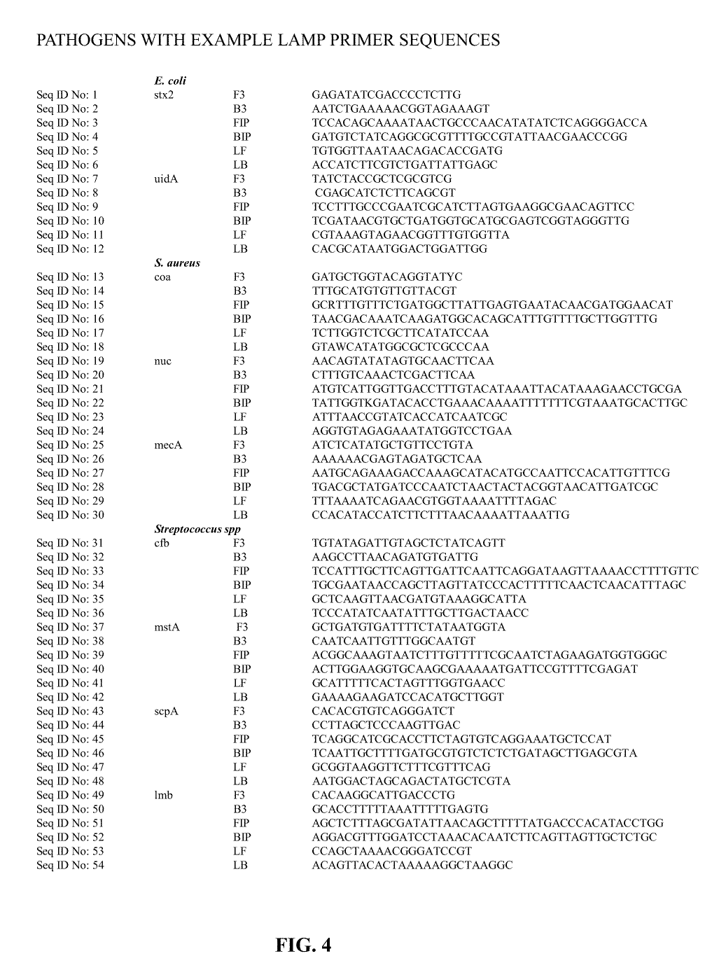

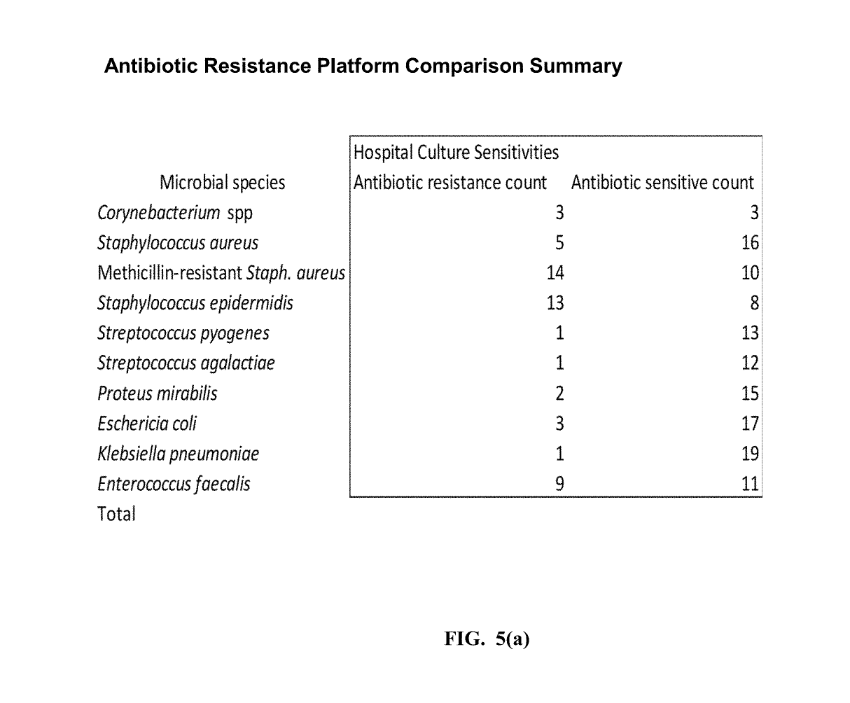

The disclosure provides a method 100, an embodiment of which is set forth in FIG. 1. The method 100 detects sepsis-related microorganisms that are present in a fluid sample. Currently, twenty microorganisms account for about 87% of sepsis-related microbial infections identified. Moreover, almost all known microbes that cause sepsis can be accounted for in a list of 50, as shown in FIG. 3. FIG. 4 includes the pathogens as identified with example LAMP primer sequences.

The method 100 begins by collecting 102 the fluid sample from a patient. A patient may be a human that has been identified with sepsis. Other animals, such as livestock, that are also susceptible to sepsis may also be “patients” for the purposes of this disclosure. The most common fluid sample that can be used is blood (e.g. collected by way of a syringe from the patient). Typically, the sample size of blood may be from one to ten milliliters but can vary depending on the size of the patient and a person of skill in the art's decision to run additional analysis.

In another embodiment, the fluid sample may be any other bodily fluid type that could be used for identification of sepsis-related microorganisms. Fluid samples from urine, cerebral spinal fluid, stool, and/or mucous membranes (i.e. mammary milk, sputum and genitourinary swab) may be utilized in order to provide more localized analysis for sepsis-related organisms. This list of fluids is not meant to be limiting and any additional fluids and or combination of fluids (i.e. aspiration from an abscess or wound) may also be used. Greater than 1 ml may be needed from each biologic fluid type. No upper limit regarding sample size theoretically exists, though approximately 3 mL is typically used for urine and blood. Smaller sample volume is generally available for wound and cerebrospinal fluid samples, though absolute concentration of pathogen is increased, allowing detection with a lower sample abundance. Blood samples are typically concentrated to 30× of reaction mixture. Urine samples are typically concentrated to 30× of reaction mixture. Sputum samples are typically diluted to 25% of reaction mixture. Stool samples are typically diluted to 2 to 5% of total reaction mixture. Wound samples are typically directly amplified from 200 μL of total reaction mixture. Cerebrospinal fluid is typically concentrated to 30× of reaction mixture.

Next, the fluid sample is fractioned 104 in order to isolate a quantity of microorganism cells. This promotes an initial concentration of cells and DNA in order to continue on with the amplification process. In one embodiment, centrifugal force is applied to the fluid sample in order to isolate the quantity of microorganisms within one of three visible fractions of the fluid sample. In another embodiment, additional filtration methods and or variations on isolating the microorganism cells are also applicable depending on the fluid sample type. An alternative method for enhanced bacterial concentration for improved detection includes use of a micropillar microfluidics peripheral filtration device. This would be expected to fractionate and concentrate microorganisms (size <3 micron diameter). Additional fractions of white blood cells, red blood cells, and plasma would be separated for possible use with other medical diagnostic tests. Another alternative method could incorporate magnetic microbeads or nanoparticles for bacterial cell extraction and concentration. Next, the method 100 includes the step of extracting 106 a portion of the microorganism cells from the fractionalized fluid sample. This is to promote optimal concentration of microorganism cells and microorganism DNA for the remaining steps within the method 100. It is expected that >1 ng of DNA per reaction well is needed for reliable and accurate detection of microorganisms.

Next, the method 100 includes the step of lysing 108 a portion of the microorganism cells extracted from the fluid sample to extract the microorganism DNA therefrom. In one embodiment, this involves heat lysis for 95° C. for 5 minutes of the extracted portion of microorganism cells to break down cell membranes and suspend the microorganism DNA within the sample fraction. Other methods are possible (mechanical, liquid homogenization, sonication, freezer-thaw).

Next, the method 100 includes the step of amplifying 110 the microorganism DNA from the microorganism cells. In one embodiment, a polymerase chain reaction (PCR) is used in order to increase the amount of DNA within the extracted sample. For example, the industrial application of the method within this disclosure may utilize isothermal loop-mediated polymerase chain reaction (LAMP) DNA amplification to accurately identify and replicate the microorganism DNA within the fluid sample. LAMP typically proceeds by high temperature isothermal amplification of a microorganism DNA template at a target temperature of from 55 to 67° C. with two to three pairs of primers used and a polymerase enzyme with high strand displacement and replicative activity (the recombinant DNA polymerase is able to displace downstream DNA encountered during synthesis, and proceeds at a rapid rate). In one embodiment, the method 100 employs four primers targeted precisely to five to six distinct regions on the gene to maximize specificity to the sepsis-related microorganisms. In another embodiment, the method 100 employs six primers related to six distinct regions on a gene to maximize the specificity to the sepsis-related microorganism. Alternate PCR techniques may also be used in order to account for lab conditions and available time-frames in conducting the method 100.

In another embodiment of the present disclosure, the amplification of the microorganism DNA is conducted until an identifiable concentration is reached. Having an identified concentration of the microorganism DNA promotes identification of the possible microorganisms within the fluid sample. In various embodiments, approximately 0.5 ng DNA/reaction is needed for successful amplification.

The industrial application of the method 100 within this disclosure may utilize a Gene-Z POC analysis machine to return data related to the positive identification of microorganism based on amplification of microbial DNA within the fluid sample by specific primers in the Gene-Z plate reaction wells. For example, data may be delivered in the form of time to threshold and estimated copy number of microorganism nucleotide sequences based on calibration curves that have been generated by lab sample serial dilution testing. Baseline signal intensity can be generated during the first 6 minutes of an amplification run. The baseline signal can then be subtracted from raw signals and the difference curves are smoothed using average signal intensity from 20 consecutive points. Dividing the threshold difference by the maximum difference then normalizes curves. Time to threshold can then be calculated as the time to normalized difference in threshold exceeding an arbitrary cut-off of 0.1.

Finally, the method 100 includes the step of amplifying 112 the microorganism DNA by a predetermined set of DNA primers to determine whether sepsis-related microorganisms are present within the fluid sample. In one embodiment, many or all sepsis-related microorganisms can be determined based on particular primers. For example, Presence of Staphylococcus aureus can be detected with LDH1 gene amplification, methicillin resistance detected by mecA amplification. Staphylococcus epidermidis can be identified by gehD with methicillin resistance detected by mecA amplification. Streptococcus agalactiae species determination can be made based on CspA2 amplification. Streptococcus pyogenes can be identified through mstA amplification. E. coli species can be identified based on amplification and determined to be nonpathogenic (O194 strain) based on stx1, stx2 and eaeA negativity or generally by pflB amplification. Klebsiella pneumoniae can be identified by Khe gene amplification. Enterococcus faecalis can be identified by BckdE1. In various embodiments, up to 50 sepsis-related microorganisms can have primers for testing through the methods of this disclosure. The primers are typically designed from a consensus of alleles for a gene unique to the microbial species. Primers targeting virulence and antibiotic resistance markers for bacterial pathogens can be designed using PrimerExplorer4 or retrieved from the literature. Additional methods of microbial signature identification include employment of open source resources such as the Tool for PCR Signature Identification (TOP SI) (http://www.bhsai.org/downloads/topsi.tar.gz), the Insignia Center for Bioinformatics and Computational Biology (http://insignia.cbcb.umd.edu). High throughput primer generation is also possible using the open-source program LAVA (LAMP Assay Veratile Analysis) (http://lava-dna.googlecode.com/) or through freely available PrimerExplorer V4 (http://primerexplorer.jp/elamp4.0.0/index.html). Primer specificity is specifically checked against the NCBI GeneBank database using NCBI BLAST. These primers can be supplied and PCR validation reactions performed according to standard protocols for both conventional RT-PCR thermocycler analysis and color-based EBT testing.

### Antibiotic Resistance Analysis:

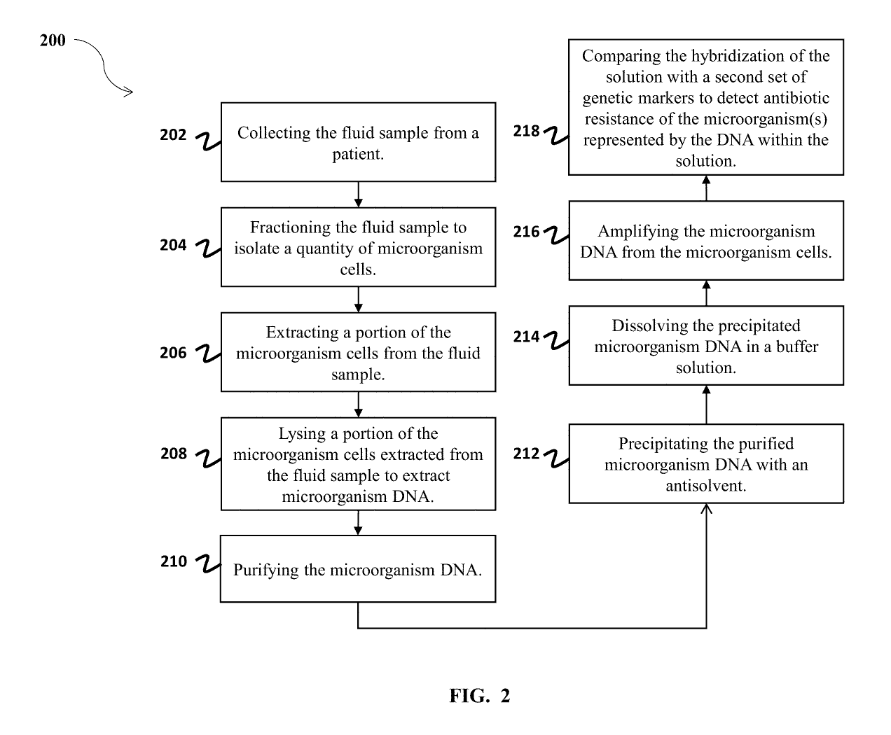

In another embodiment of the present disclosure, e.g. as shown in FIG. 2, the disclosure describes an alternate improved method 200 for detecting whether antibiotic-resistant sepsis-related microorganisms are present in a fluid sample.

The method 200 includes the steps of collecting 202 the fluid sample from a patient (as described at step 102 above); fractioning 204 the fluid sample to isolate a quantity of microorganism cells (as described at step 104 above); extracting 206 a portion of the microorganism cells from the fluid sample (as described at step 106 above); and lysing 208 a portion of the microorganism cells extracted from the fluid sample to extract microorganism DNA (as described at step 108 above). Any one or more of these steps may be the same or different from those described above.

Then, the method 200 further includes the step of purifying 210 the microorganism DNA. In one embodiment this is done through a phenol cholorform extraction (by mixing the sample with equal volumes of a phenol chloroform mixture), in order to concentrate the nucleic acids and reduce the presence of proteins attached to the microorganism DNA from the fluid solution.

Next, the method 200 includes the step of precipitating 212 the purified microorganism DNA with an antisolvent. In one embodiment ethanol is used as the antisolvent. This step forms precipate from the purified solution containing a higher concentration of the microorganism DNA for analysis.

Next, the method 200 continues by dissolving 214 the precipitated microorganism DNA in a buffer solution. In one embodiment, the buffer solution is 50 μl of Tris-EDTA (TE) buffer. The quantity and particular buffer may vary in based on the current conditions. Particularly, other fluid sample types may include additional purification steps as well as other buffers, such as phosphate buffered saline, in order to effectively dissolve the extracted microorganism DNA.

Next, the method 200 amplifies 216 the microorganism DNA from the microorganism cells. This is analogous to step 110 above, but may include additional or different steps as well as appreciated by those of skill in the art.

Next, the method 200 further includes the step of amplification and hybridization of 218 a nucleotide sequence of the extracted microorganism DNA. In another embodiment, this step is conducted through the use of a parallel PCR method. Other PCR methods may also be available for use during this step in the method 200.

Finally, the method 200 further includes the step of amplifying 220 the solution and hybridization to a predetermined second set of genetic markers in order to detect the antibiotic resistance genes of the microorganism represented by the DNA within the solution. In one embodiment, the predetermined second set of genetic markers includes a plurality of antibiotic resistance genes found within sepsis-related microorganisms. In the industrial application of method 200, antibiotic-specific resistant genes most relevant to the hospital setting can be determined by profiling either the Antibiotic Resistant Gene Database with the OpenArray PCR system, or the WaferGen platform (http://www.wafergen.com/applications/gene-expression-profiling/) or creating an additional database from obtained results over time. Each sample is tested in technical triplicates. If at least two of the assays are positive, the gene will be determined as present. Resistance gene profiles will be analyzed, interrogating for resistance to certain antibiotics or classes of antibiotics in an effort to identify the drug resistance profile.

In one embodiment, the present disclosure directly addresses the need for fast and accurate diagnosis of offending pathogens in the diagnosis of sepsis. In another embodiment, the present disclosure directly addresses the need for fast and accurate diagnosis of offending pathogens by adapting a POC device to the diagnosis of sepsis. Synergistic implementation of both methods (100 and 200) can enable physicians to identify the microorganisms responsible for a patient's septic state in 20 to 30 minutes rather than three days, and reveal an organism's genetic weaknesses in seven hours. This will maximize antibiotic utility, eradicate infection, and help conserve important antibiotics by eliminating the guesswork involved in treating septic patients.

It should be noted that the timeframes mentioned are not meant to be limiting. Although the times of 20 to 30 minutes and 7 hours are used here, the disclosure should not be restricted to any specific time period at this time, but should be viewed as changing the range from several days to a first step in a relatively short waiting time followed by a comprehensive analysis in another longer waiting time, but still relatively shorter than several days. Further, the molecular analyses conducted through these methods tend to be both more accurate and more sensitive than culture-based analysis.

FIG. 3 is a representative table listing the 50 sepsis-related microorganisms that may be identified using method 100. Along with each microorganism is also the associated genetic marker(s) that are used to identify the particular microorganism with the fluid sample. It should be noted that this list is not meant to limiting and can be modified in order to account for additional, relevant microorganisms. As discussed above, FIG. 4 indicates the pathogens as identified with example LAMP primer sequences.

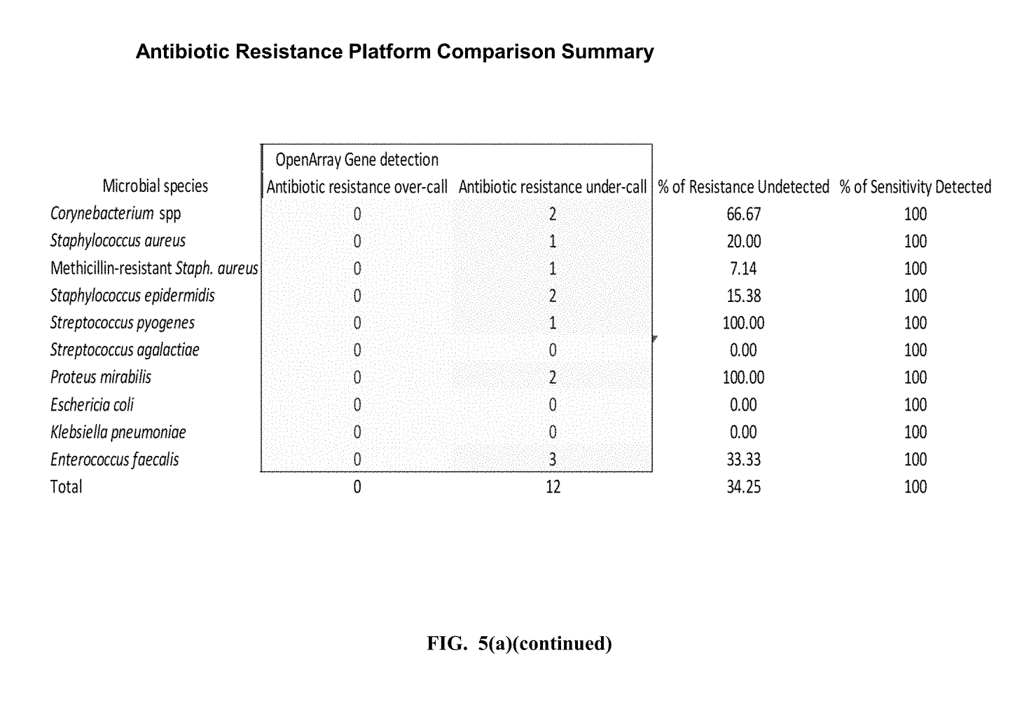

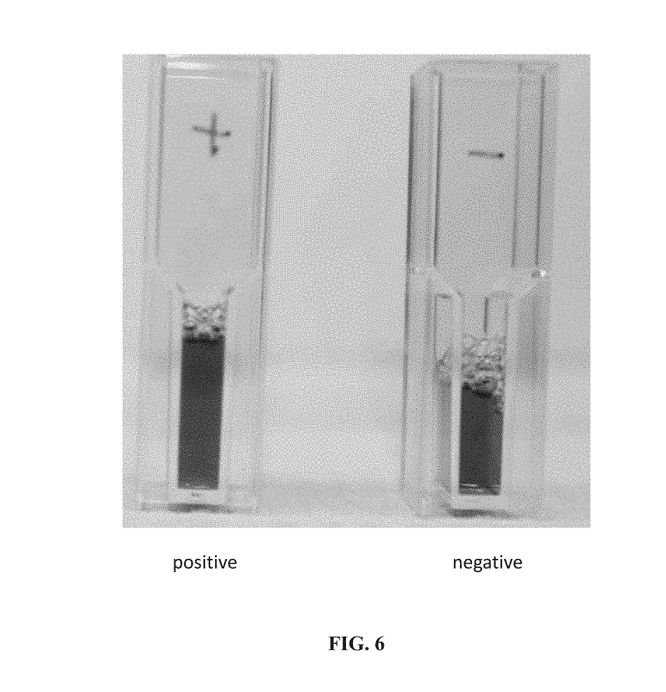

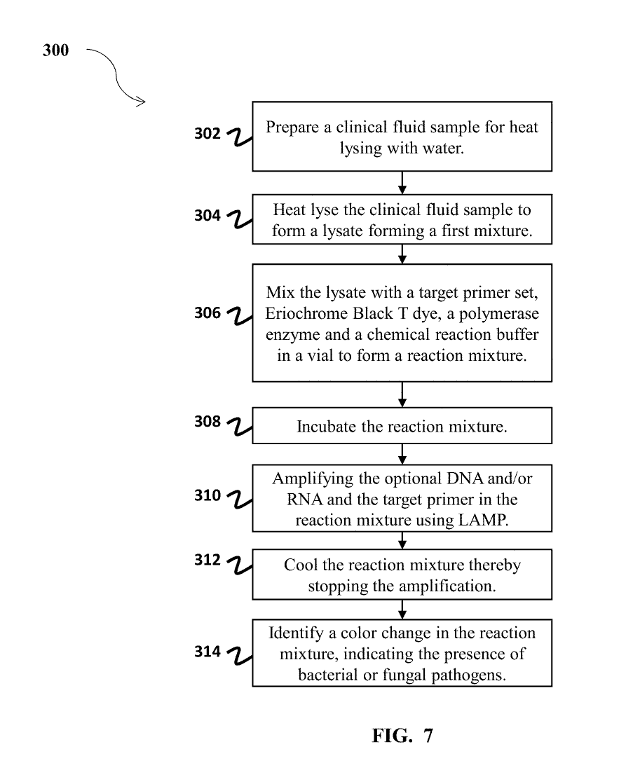

FIG. 5 is a representative output table of the antibiotic resistance analysis conducted through method 200. Along with each identified microorganism are their known antibiotic resistance count, their antibiotic sensitivity count, as well as the analysis and identification of antibiotic gene resistance markers. The identified markers as compared to the prior columns are compared in order to give a percentage of resistance undetected by the identified microorganism.

### A Method for Detecting Bacterial and Fungal Pathogens:

In various embodiments, the methodologies for direct amplification of DNA and RNA sequences to target genetic regions of interest can allow rapid discrimination of microbial pathogens in a point-of-care time-frame. By utilizing either fluorescence detection by a real-time PCR instrument or naked-eye visual detection of color change from purple to blue using EBT-dye chelation (as illustrated in FIG. 6 and described herein), the direct amplification methods used here offer diagnostic capabilities that are improved compared to the gold standards of clinical microbial pathogen identification, but in a significantly reduced time frame and with lower resource use.

The methods described herein typically utilize filtration to rapidly concentrate bacteria in sample matrices with lower bacterial loads and direct LAMP amplification without DNA purification from clinical blood, urine, mucocutaneous swab/wound, sputum and stool samples, that takes 10 to 35, 35 to 120, 60 to 120, or 30 to 60, or from 45 to 90 minutes to the final results. The methods described below can be tested using at least two methods of detection: 1) visual discrimination of color change in the presence of Eriochrome Black T (EBT) dye following amplification and 2) real-time thermocycler fluorescent detection of LAMP amplification.

### EBT Dye Method:

In one embodiment of the present disclosure, a method 300 for detecting bacterial and fungal pathogens at clinically relevant concentrations in a clinical fluid sample using a visual color change test is illustrated in FIG. 7 and described herein.

In another embodiment, the method 300 includes the steps of: preparing the clinical fluid sample for heat lysing with water 302; heat lysing the clinical fluid sample to form a lysate that optionally includes bacterial and/or fungal DNA and RNA forming a first mixture 304; mixing the lysate with a target DNA primer and/or target RNA primer, Eriochrome Black T dye, a polymerase enzyme and a chemical reaction buffer in a vial to form a reaction mixture 306; incubating the reaction mixture 308; amplifying the optional bacterial and/or fungal DNA and RNA and the target DNA primer and/or the target RNA primer in the reaction mixture using loop mediated isothermal amplification 310; cooling the reaction mixture for a predetermined amount of time thereby stopping the amplification 312; and identifying a color change in the reaction mixture which is indicative of the presence of bacterial or fungal pathogens in the clinical fluid sample at the clinically relevant concentrations 314.

In another embodiment, the step of preparing the clinical fluid sample can include filtrating the clinical fluid sample to rapidly concentrate bacteria in the clinical fluid sample. The filtrating step also applies to methods 400 and 500 described herein.

### Thermocycler Method:

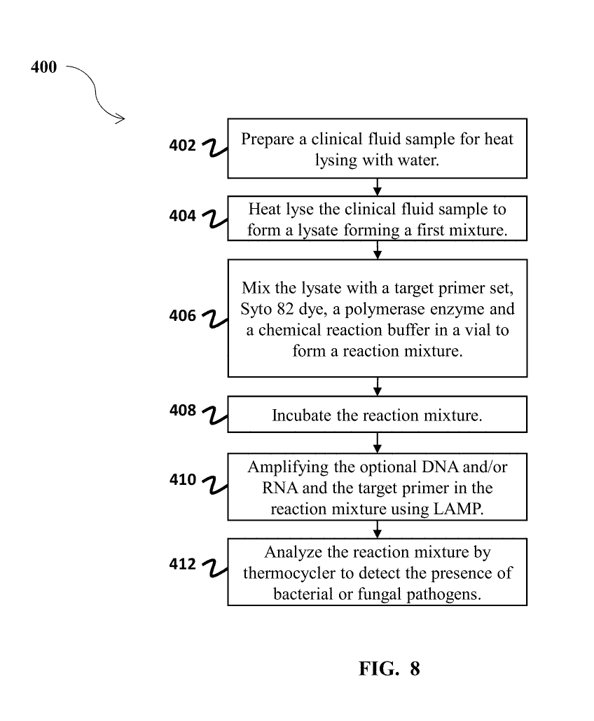

In yet another embodiment, the present disclosure provides a method 400 for detecting bacterial and fungal pathogens at clinically relevant concentrations in a clinical fluid sample (as shown in FIG. 8).

In one embodiment, the method 400 includes the steps of: preparing the clinical fluid sample for heat lysing with water 402; heat lysing the clinical fluid sample to form a lysate that optionally includes bacterial and/or fungal DNA and RNA forming a first mixture 404; mixing the lysate with a target DNA primer and/or a target RNA primer, Syto 82 dye, a polymerase enzyme, and a chemical reaction buffer in a vial to form a reaction mixture 406; incubating the reaction mixture 408; amplifying the optional bacterial and/or fungal DNA and/or RNA and the target DNA primer and/or the RNA primer in the reaction mixture using loop mediated isothermal amplification 410; and analyzing the reaction mixture by thermocycler to detect the presence of bacterial or fungal pathogens in the clinical fluid sample at the clinically relevant concentrations 412.

### EBT Dye and Thermocycler Method:

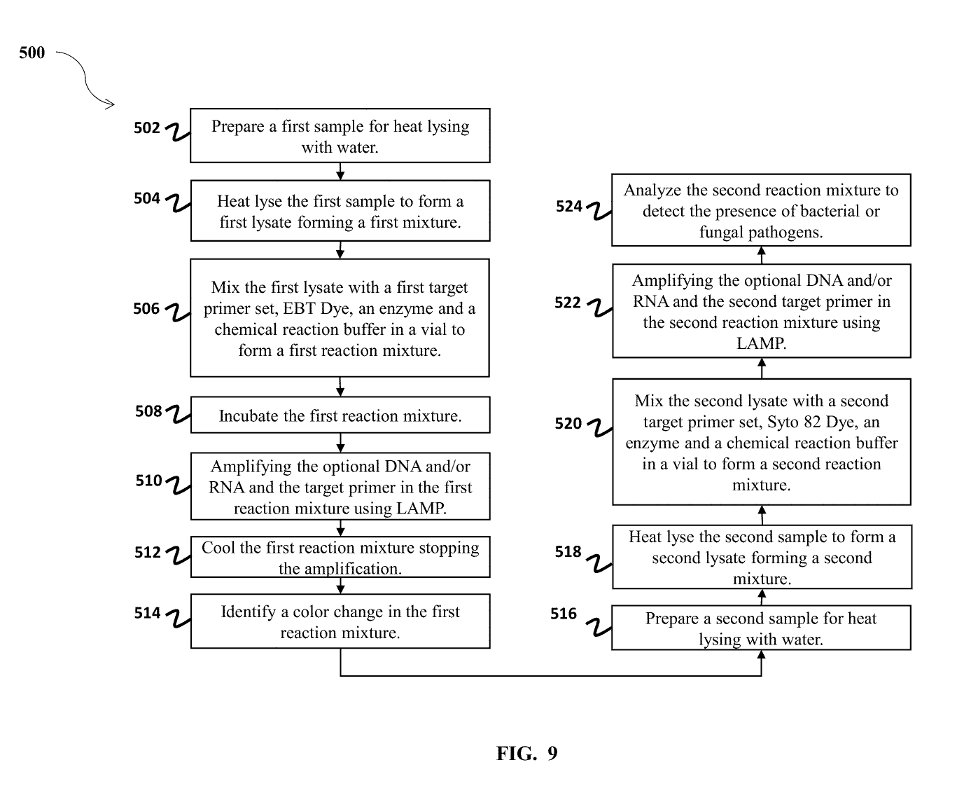

In still another embodiment, the present disclosure provides a method 500 for detecting bacterial and fungal pathogens at clinically relevant concentrations in clinical fluid samples using a visual color change test and a fluorescence test, as illustrated in FIG. 9.

In a third embodiment, the method 500 includes the steps of: preparing a first clinical fluid sample for heat lysing with water 502; heat lysing the first clinical fluid sample to form a first lysate that optionally includes bacterial and/or fungal DNA and/or RNA forming a first mixture 504; mixing the first lysate with a target DNA primer and/or a target RNA primer, Eriochrome Black T dye, a polymerase enzyme, and a chemical reaction buffer in a vial to form a first reaction mixture 506; incubating the first reaction mixture 508; amplifying the optional bacterial and/or fungal DNA and or RNA and the target DNA primer and/or the target RNA primer in the first reaction mixture using loop mediated isothermal amplification 510; cooling the first reaction mixture for a predetermined amount of time thereby stopping the amplification 512; identifying a color change in the first reaction mixture which is indicative of the presence of bacterial or fungal pathogens in the first clinical fluid sample at the clinically relevant concentrations 514; preparing a second clinical fluid sample for heat lysing with water 516; heat lysing the second clinical fluid sample to form a second lysate that optionally includes bacterial and/or fungal DNA and/or RNA forming a second mixture 518; mixing the second lysate with a second target DNA primer and/or a second target RNA primer, Syto 82 dye, a polymerase enzyme, and a chemical reaction buffer in a vial to form a second reaction mixture 520; amplifying the optional bacterial and/or fungal DNA and/or RNA and the second target DNA primer and/or the second target RNA primer in the second reaction mixture using loop mediated isothermal amplification 522; and analyzing the second reaction mixture by thermocycler to detect the presence of bacterial or fungal pathogens in the second clinical fluid sample at the clinically relevant concentrations 524.

### Method for Preparing a Shelf Stable Target Primer:

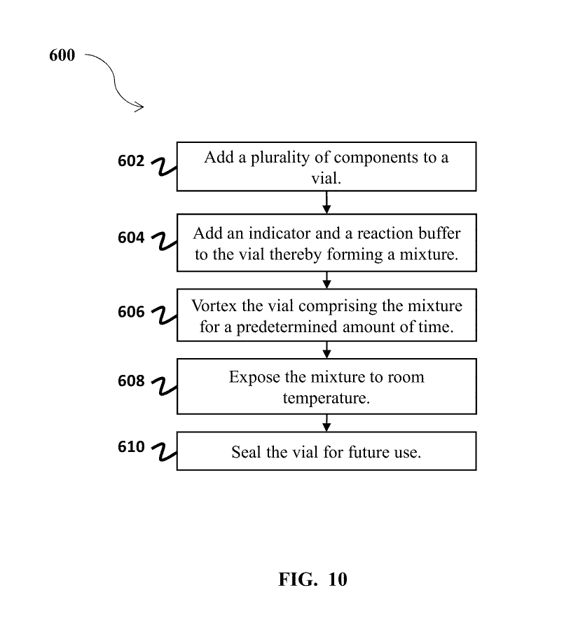

The disclosure provides a method 600 for improving a shelf stable target DNA/RNA primer for the detection of bacterial and fungal pathogens at clinically relevant concentrations in a clinical fluid sample, as illustrated in FIG. 10.

In one embodiment, the method 600 includes the steps of: adding a plurality of components to a vial 602, where the components are chosen from a predetermined amount of: trehalose, polymerase, a primer mix, glycerol, a surfactant, a serum albumin, dNTP, and magnesium sulfate; adding an indicator and a reaction buffer to the vial thereby forming a mixture 604; vortexing the vial including the mixture for a predetermined amount of time 606; exposing the mixture to room temperature ±5° C. for 24 hours 608; and sealing the vial for future use of the detection of bacterial and fungal pathogens at clinically relevant concentrations in a clinical fluid sample 610.

### Bacterial and Fungal Pathogens:

The bacterial and fungal pathogens can be any known in the art. In one embodiment, the bacterial and fungal pathogens are chosen from Escherichia coli, Staphylococcus aureus, Enterococcus faecalis, Enterococcus faecium, Streptococcus pyogenes, Pseudomonas aeruginosa, Klebsiella pneumonia, Proteus mirabilis, Staphylococcus epidermidis, Streptococcus agalactiae, Candida albicans, Enterococcus casseliflavus, Enterococcus gallinarum, Clostridium difficile. Moreover, any one of more of these pathogens may be focused on using the methods of this disclosure.

In another embodiment, the methods for detecting bacterial and fungal pathogens can detect mecA methicillin resistance gene that is found in bacterial cells. The mecA methicillin resistance gene allows a bacterium to be resistant to antibiotics such as methicillin, penicillin and other penicillin-like antibiotics. The most common carrier of the mecA gene is the bacterium known as MRSA. The mecA gene can also be found in Streptococcus pneumonia and other strains of microorganisms resistant to antibiotics.

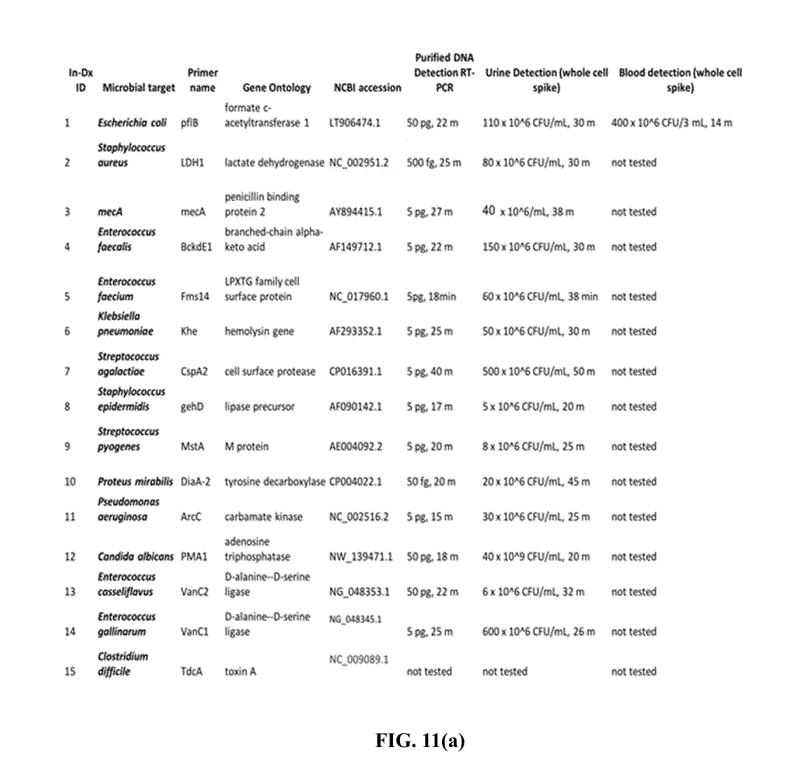

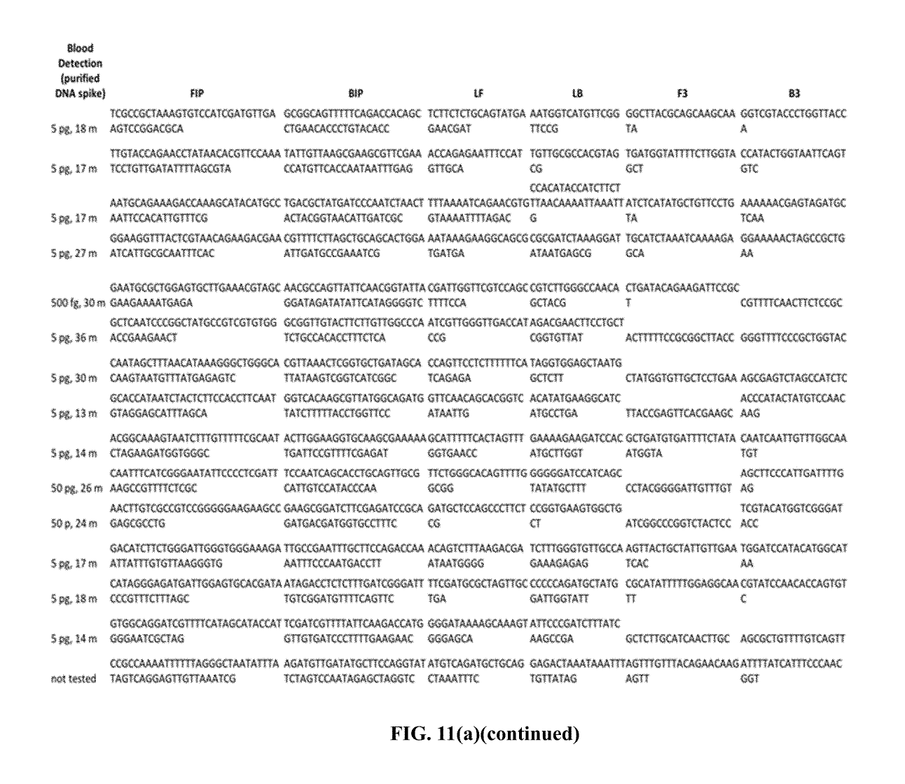

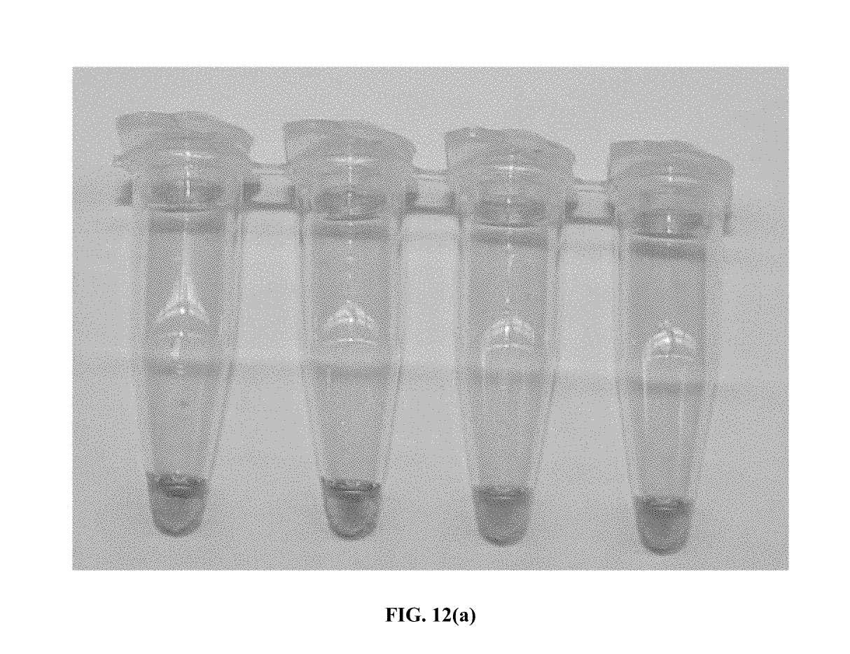

In various embodiments, single gene targets can be selected for comparative quantification of pathogens to enable to effectively rule in or rule out the presence of each pathogen (e.g. those shown in FIG. 11). Primers can be designed to be exclusive to one organism of interest except for mecA, which is a generalized antibiotic resistance gene target with known associations to a number of clinical pathogens including S. aureus and S. epidermidis, a coagulase negative Staphylococcus species, which has been clinically recognized as a commensal skin contaminant, an opportunistic pathogen, and potential microbial gene-transfer reservoir.

Preparing the Clinical Fluid Sample for Heat Lysing with Water:

The clinical fluid sample is not particularly limited and may be of any type known in the art. In one embodiment, the clinical fluid sample is a clinical blood sample, a clinical urine sample, a clinical mucocutaneous swab/wound sample, a clinical sputum sample, a clinical stool sample and/or a clinical cerebrospinal culture sample.

First introduced above, each of the methods 300, 400, 500, 600, includes the step of filtrating the clinical fluid sample before preparing the sample for heat lysing with water. The amount of the clinical fluid sample may be 2 to 4, or 3 to 5, or 4 to 6, 5 to 7, or 6 to 8, mL per each clinical sample. In addition, the clinical fluid sample may be stored before preparation at a temperature of from 3 to 5, or 4 to 6, or from 5 to 7, ° C. For the preparation of the clinical fluid sample, the sample may go through a filtration/concentration system. For example, a blood or urine sample may be filtrated through an EconoSpin spin Column filter tube for 8 to 10, or 9 to 11, or 10 to 12, minutes. Blood may variably be extracted with magnetic nanoparticles. Other clinical fluid samples may be collected from the initial container and are ready to begin the preparation for heat lysing. Next, a liquid can be added to the collection tube including the sample, wherein the liquid can be but is not limited to water. The sample is then aspirated from the collection tube and transferred by a pipette to an Eppendorf tube or a tube of similar design. The tube including the liquid and the sample may then be heated on a heating block of from 80 to 100, or 90 to 110, or 100 to 120, ° C. Last, the step of heating of the filtrate may be completed at any time from 5 to 15, or 10 to 20, or 15 to 25, minutes.

### Heat Lysing the Clinical Fluid Sample to Form a Lysate Forming a First Mixture:

As first introduced above, each of the methods 300, 400, 500, 600, includes the step of heat lysing the clinical fluid sample to form a lysate that optionally comprises bacterial and/or fungal DNA and RNA forming a first mixture. The step of heat lysing may be completed in any way known in the art. For example, the step of heat lysing may be completed at any time from 5 to 15, from 10 to 20, or from 15 to 25, minutes. Similarly, the step of heat lysing may be completed at a temperature of from 85 to 105, from 90 to 110, or from 95 to 115, ° C. Any one or more portions of the lysate may be combined to form the reaction mixture. Typically, an amount of the lysate of from 100 μL to 500 μL, from 200 μL to 1 mL, or from 1 mL to 5 mL forms the first mixture. It is also contemplated that the method may utilize one or more variables outside of the aforementioned ranges. Moreover, the apparatus used to heat lyse the clinical fluid sample may be any known in the art.

Lysis is the process by which a cell membrane is opened up to release its genetic material. Generally, lysing occurs by adding a chemical to the sample creating a mixture and heating the mixture. In one embodiment, lysis can occur by mechanical methods (e.g. glass or ceramic beads, sonication, and/or freezing), or through high temperatures (heat lysis) which disrupts the bonds within the cell walls or by acid-base disruption by dilute acids of pH<7.2 or bases of pH >7.8. In addition, lysis naturally occurs through enzymes or through organic solvents like alcohols, ether or chloroform. It is also contemplated that the method may utilize one or more variables outside of the aforementioned ranges. Moreover, the apparatus used to heat lyse the clinical fluid sample may be any known in the art.

Mixing the Lysate with a Target DNA Primer and/or Target RNA Primer, an Indicator, a Polymerase Enzyme and a Chemical Reaction Buffer in a Vial to Form a Reaction Mixture:

Each of the methods 300, 400, 500, 600, also includes the step of mixing the lysate with a target DNA primer and/or target RNA primer, an indicator, a polymerase enzyme, and a chemical reaction buffer in a vial to form a reaction mixture. The step of mixing the lysate may be completed in any way known in the art. The lysate itself is not particularly limited and may be any formed from the aforementioned step of heat lysing. The step of mixing may be any known in the art and typically includes combining the lysate, the target DNA primer and/or target RNA primer, the indicator, the polymerase enzyme, and the chemical reaction. The reaction mixture may also include a reagent that is also not particularly limited and may include or be acetic acid, chloroform, or formic acid. Any one or more portions of the lysate may be combined with any one or more portions of the target DNA primer and/or target RNA primer, the indicator, the polymerase enzyme, and the chemical reaction to form the reaction mixture. The volume of the lysate, the target DNA primer and/or target RNA primer, the indicator, the polymerase enzyme, and the chemical reaction buffer, and the first mixture is not particularly limited. The reaction mixture is not limited and may be any mixture formed from the aforementioned step of heat lysing forming a first mixture. The step of mixing may be any known in the art and typically includes pipetting the reaction mixture into a vial and vortexing the vial. The target DNA and/or RNA primer is also not particularly limited and may include or be any combination of DNA and/or RNA necessary. Similarly, the indicator is not particularly limited to and may include or be Eriochrome Black T Dye (EBT) dye, Syto-82 Dye, or Azo Dye. EBT Dye may include any complexometric indicator that is used in complexometric titrations. EBT dye is also known as but not limited to azo dye. Syto-82 is an orange fluorescent nucleic acid stain that exhibits bright, orange fluorescence upon binding to nucleic acids. The buffers are also not particularly limited to and may include or be an enzymatic buffer or a reaction stabilizer element such as Pluronic F68. Similarly, the polymerase enzyme is also not particularly limited to and may include or be DNA polymerase, RNA polymerase, or any combination of DNA/RNA polymerase. Any one or more portions or amount of the first mixture may be combined with any one or more portions of amount of the target DNA and/or RNA primer, any portion or amount of the indicator, any portion or amount of the polymerase enzyme and any amount of the reaction buffers to form the reaction mixture.

The volume of the reaction mixture including the first mixture, the target DNA and/or RNA primer, the indicator and the reaction buffer is not particularly limited. Typically, an amount of the first mixture of from 100 μL to 500 μL, from 200 μL to 1 mL, or from 1 mL to 5 mL includes an amount of the lysate of from 100 μL to 500 μL, from 200 μL to 1 mL, or from 1 mL to 5 mL, is optionally combined with an amount of the reagent of from 100 μL to 500 μL, from 200 μL to 1 mL, or from 1 mL to 5 mL, is combined with an amount of the chemical reaction buffer of from 100 μL to 500 μL, from 200 μL to 1 mL, or from 1 mL to 5 mL, an amount of the target DNA and/or RNA primer of from 100 μL to 500 μL, from 200 μL to 1 mL, or from 1 mL to 5 mL, is combined with an amount of the indicator of from 100 μL to 500 μL, from 200 μL to 1 mL, or from 1 mL to 5 mL is combined with an amount of the polymerase enzyme of from 100 μL to 500 μL, from 200 μL to 1 mL, or from 1 mL to 5 mL, to form the reaction mixture. It is also contemplated that the method may utilize one or more variables outside of the aforementioned ranges as this operation is scalable depending upon the number of targets and desired time to detection. Moreover, the apparatus used to mix the lysate to form a reaction mixture may be any known in the art.

### Incubating the Reaction Mixture:

Both of the methods 300 and 500, also includes the step of incubating the reaction mixture. The step of incubating may be completed in any way known in the art. For example, the step of incubating may be completed at any time from 0 to 20, from 10 to 20, from 20 to 30, or from 40 to 50, minutes. Similarly, the step of incubating may be completed at a temperature of from 50 to 60, from 60 to 70, or from 70 to 80, ° C. It is also contemplated that the method may utilize one or more variables outside of the aforementioned ranges. Moreover, the apparatus used to incubate the reaction mixture may be any known in the art.

Amplifying the Optional Bacterial and/or Fungal DNA and RNA and the Target DNA Primer and/or the Target RNA Primer in the Reaction Mixture Using Loop Mediated Isothermal Amplification:

As first introduced above, each of the methods 300, 400, 500, 600, includes the step of amplifying the optional bacterial and/or fungal DNA and RNA and the target DNA primer and/or the target RNA primer in the reaction mixture using loop mediated isothermal amplification (LAMP). The step of amplifying the optional bacterial and/or fungal DNA and RNA and the target DNA primer and/or the target RNA primer in the reaction mixture using LAMP may be completed in any way known in the art. For example, the step of amplifying the optional bacterial and/or fungal DNA and RNA and the target DNA primer and/or the target RNA primer in the reaction mixture using LAMP may be completed at any time from 0 to 10, 10 to 20, 20 to 30, from 30 to 40, or from 40 to 50, minutes. Similarly, the step of amplifying the optional bacterial and/or fungal DNA and RNA and the target DNA primer and/or the target RNA primer in the reaction mixture using LAMP may be completed at a temperature of from 55 to 60, from 60 to 65, or from 65 to 70, ° C. It is also contemplated that the method may utilize one or more variables outside of the aforementioned ranges. Moreover, the apparatus used to amplify the optional bacterial and/or fungal DNA and RNA and the target DNA primer and/or the target RNA primer in the reaction mixture using LAMP may be any known in the art.

### Cooling the Reaction Mixture for a Predetermined Amount of Time Thereby Stopping the Amplification:

First mentioned above, both of the methods 300 and 500, include the step of cooling the reaction mixture for a predetermined amount of time thereby stopping the amplification. The step of immersing the reaction may be completed in any way known in the art. For example, the step of cooling the reaction mixture for a predetermined amount of time thereby stopping the amplification may be completed at any time from 45 to 50, from 50 to 60, or from 60 to 70, seconds. Similarly, the step of cooling the reaction mixture for a predetermined amount of time thereby stopping the amplification may be completed at a temperature of from 2 to 4, from 4 to 8, or from 8 to 10, ° C. The cooling of the reaction mixture may be completed by immersing the reaction mixture in an ice. A cooling bath or an ice bath is a liquid mixture, which is used to maintain a low temperature. The low temperatures are used to perform a chemical reaction below room temperature. The addition of a cooling step is not necessary for identification of color change after incubation at the predetermined time steps but will improve product visualization in incubation chambers (tubes). A refrigerator or freezer or other cooling unit can also accomplish the reaction termination. It is also contemplated that the method may utilize one or more variables outside of the aforementioned ranges. Moreover, the apparatus used to a cooling bath or an ice bath is a liquid mixture, which is used to maintain a low temperature. The low temperature are used to perform a chemical reaction below room temperature may be any known in the art.

Identifying a Color Change in the Reaction Mixture which is Indicative of the Presence of Bacterial or Fungal Pathogens in the Clinical Fluid Sample at the Clinically Relevant Concentrations:

As first introduced above, both of the methods 300 and 500, includes the step of identifying a color change in the reaction mixture which is indicative of the presence of bacterial or fungal pathogens in the clinical fluid sample at the clinically relevant concentrations. The step of identifying a color change in the reaction mixture may be completed in any way known in the art. For example, the step of identifying a color change in the reaction mixture may be completed at any time on 5 minute intervals from 0 to 5, from 5 to 10, 10 to 15, 15 to 20, 20 to 25, 25 to 30, 30 to 35, 35 to 40, or from 40 to 45, minutes. Similarly, the step of identifying a color change in the reaction mixture may be completed at a temperature of from 55 to 60, from 60 to 65, or from 65 to 70, ° C. The step of identifying a color change is not particularly limited to a particular method but may include or be a non-color blind human, a spectrophotometer, or any instrument that may read particular wavelengths. The step of identifying a color change in the reaction using a spectrophotometer may be completed at a wavelength of from 360 to 380, 380 to 400, 400 to 420, 420 to 440, 440 to 460, 460 to 470, or from 470 to 480, nm. It is also contemplated that the method may utilize one or more variables outside of the aforementioned ranges. Moreover, the apparatus used to identify a color change in the reaction mixture may be any known in the art.

### Analyzing the Reaction Mixture by Thermocycler to Detect the Presence of Bacterial or Fungal Pathogens in the Clinical Fluid Sample at the Clinically Relevant Concentrations:

As first introduced above, each of the methods 300, 400, 500, 600, includes the step of analyzing the reaction mixture by thermocycler to detect the presence of bacterial or fungal pathogens in the clinical fluid sample at the clinically relevant concentrations. The step of analyzing the reaction mixture by thermocycler may be completed in any way known in the art. For example, the step of analyzing the reaction mixture by thermocycler may be completed at any time from 30 to 40, from 40 to 50, or from 50 to 60, minutes. Similarly, the step of analyzing the reaction mixture by thermocycler may be completed at a temperature of from 50 to 60, from 60 to 70, or from 70 to 80, ° C. It is also contemplated that the method may utilize one or more variables outside of the aforementioned ranges. Moreover, the apparatus used to analyze the reaction mixture by thermocycler may be any known in the art.

### Adding a Plurality of Components to a Vial:

As described above, each of the methods 300, 400, 500, 600, includes the step of adding a plurality of components to a vial. The step of adding a plurality of components to a vial may be completed in any way known in the art. For example, the step of adding a plurality of components to a vial may be completed at any time from 0 to 20, from 10 to 20, or from 20 to 30, or from 40 to 50 minutes. Similarly, the step of adding a plurality of components to a vial may be completed at a temperature of from 1 to 35, from 5 to 35, or from 15 to 35, ° C. The plurality of components includes at least two components. In addition, the components themselves are not particularly limited to and may include or be a predetermined amount of trehalose, polymerase, a primer mix, glycerol, a surfactant, a serum albumin, a nucleoside triphosphate, or magnesium sulfate. Any one or more portions of the components may be combined with any one or more portions of at least one other component to form the plurality of components. The polymerase is also not particularly limited and may include or be DNA polymerase, RNA polymerase, or any other polymerase. Similarly, the primer mix is also not particularly limited and may include or be 10× Isothermal Amplification Buffer (New England Biolabs). The surfactant is also not particularly limited and may include or be Pluonic F-68. In addition, the serum albumin is also not particularly limited and may include or be Bovine Serum Albumin or other mammalian forms. Similarly, the nucleoside triphosphate is also not particularly limited and may include or be adenosine triphosphate, guanosine triphosphate, cytidine triphosphate, 5-methyluridine triphosphate, or uridine triphosphate. The volume of the plurality of components is not particularly limited. Typically, an amount of the plurality of components is from 1 μL to 10 μL, from 5 μL to 100 μL, or from 50 μL to 5 mL. The step of adding a plurality of components may be any known in the art. It is also contemplated that the method may utilize one or more variables outside of the aforementioned ranges. Moreover, the apparatus used to add a plurality of components to a vial may be any known in the art.

### Adding an Indicator and a Reaction Buffer to the Vial Thereby Forming a Mixture:

As first introduced above, each of the methods 300, 400, 500, 600, includes the step of adding an indicator and a reaction buffer to the vial thereby forming a mixture. The step of adding an indicator and a reaction buffer may be completed in any way known in the art. The components in the vial itself are not particularly limited and may be any combination formed from the aforementioned step of adding a plurality of components to the vial and vortexing the vial. The step of adding an indicator and a reaction buffer to the vial forming a mixture may be any known in the art and typically includes adding or combining the indicator and the reaction buffer to the vial. The indicator is also not particularly limited and may include or be EBT Dye, Syto-82 Dye, or any other dye for detecting bacterial and fungal pathogens in clinical fluid samples. Similarly, the reaction buffer is also not particularly limited and may include or be optimized for polymerase enzyme reactions such as 10× Isothermal Amplification Buffer (New England Biolabs). Any one or more portions of the indicator may be combined with any one or more portions of the reaction buffer to form a mixture. The volume of the plurality of components, the indicator, and the reaction buffer is not particularly limited. Typically, an amount of the indicator of from 1 to 50, from 15 to 50, or from 25 to 50, mL is combined with an amount of the reaction buffer of from 1 to 50 or from 10 to 1000 μL, or from 50 μL to 5 mL, to form the first mixture. In addition, the step of adding an indicator and a reaction buffer to the vial forming a mixture may be completed at any time from 1 to 60, from 20 to 60, or from 30 to 60, minutes. Similarly, the step of adding an indicator and a reaction buffer to the vial forming a mixture may be completed at a temperature of from 1 to 35, from 15 to 35, or from 25 to 35, ° C. It is also contemplated that the method may utilize one or more variables outside of the aforementioned ranges. Moreover, the apparatus used to add an indicator and a reaction buffer to the vial forming a mixture may be any known in the art.

### Vortexing the Vial Comprising the Components for a Predetermined Amount of Time:

As first introduced above, each of the methods 300, 400, 500, 600, includes the step of vortexing the vial comprising the components for a predetermined amount of time. The step of vortexing the vial may be completed in any way known in the art. For example, the step of vortexing the vial may be completed at any time from 2 to 5, from 5 to 10, or from 10 to 15, seconds. Similarly, the step of vortexing the vial may be completed at a temperature of from 1 to 35, from 5 to 35, or from 15 to 35, ° C. It is also contemplated that the method may utilize one or more variables outside of the aforementioned ranges. Moreover, the apparatus used to vortex the vial comprising the components for a predetermined amount of time may be any known in the art.

### Exposing the Mixture to Room Temperature:

As described above, each of the methods 300, 400, 500, 600, includes the step of exposing the mixture to room temperature. The step of exposing the mixture to room temperature may be completed in any way known in the art. For example, the step of exposing the mixture to room temperature may be completed at any time from 12 to 24, from 24 to 36, or from 36 to 48, hours. Similarly, the step of exposing the mixture to room temperature may be completed at a temperature of from 2 to 5, from 5 to 10, or from 10 to 12, ° C. In addition, the step of exposing the mixture to room temperature may be completed at a temperature ±5° C. It is also contemplated that the method may utilize one or more variables outside of the aforementioned ranges. Moreover, the apparatus used to expose the mixture to room temperature may be any known in the art.

Sealing the Vial that Comprises the Components:

As first introduced above, each of the methods 300, 400, 500, 600, includes the step of sealing the vial that comprises the components. The step of sealing the vial may be completed in any way known in the art. For example, the step of sealing the vial may be completed at any time from 0 to 1000, from 0 to 2000, or from 0 to 3000, minutes. Similarly, the sealing the vial comprising the components may be completed at a temperature of from 1 to 35, from 5 to 35, or from 15 to 35, ° C. The step of sealing the vial may be completed to further prepare the vial for bacterial and fungal pathogen detection, but the vial may also be sealed for future use from up to 5 to 10 days, 10 to 12 days, or from 12 to 14 days. It is also contemplated that the method may utilize one or more variables outside of the aforementioned ranges. Moreover, the apparatus used to seal the vial comprising the components may be any known in the art.

### EXAMPLES

A series of clinical fluid samples were obtained from numerous different patients over the course of months. These clinical fluid samples were of many different types, as is described in greater detail below. Moreover, their volumes were also of different sizes, as is also described in detail below. These clinical samples were analyzed using various embodiments of the methods described herein and in greater detail below.

In total, 239 samples were collected across culture types (31 blood, 122 urine, 73 mucocutaneous wound/swab, 11 sputum and two stool samples) from 229 consecutively enrolled patients with suspected clinical infection with samples analyzed both at the hospital and by one embodiment of this disclosure. The overall sensitivity of the EBT method and the Thermocycler method was 76.4% with a specificity of 98.3%. The positive predictive value for all samples was 63.5% and negative predictive value was 99.1%. The results indicate the LAMP-based embodiments allows rapid and precise diagnosis of clinical infections by targeted pathogens across multiple culture types for point-of-care utilization.

A multicenter evaluation of prospectively collected clinical samples across different culture types are performed in a 96-well plate format utilizing: (1) human visual discrimination of color changes after sample lysis and reaction incubation at 63° C. in the presence of Eriochrome Black T (EBT) dye and/or (2) LAMP isothermal real-time polymerase chain reaction (RT-PCR). Either method allows detection of bacterial and fungal pathogens at clinical relevant concentrations after minimal processing with 45 to 50 minutes. Duplicate clinical samples collected from prospectively-enrolled patients with suspected infection analyzed by the at least two methods were compared to hospital testing for detection of the targeted clinical pathogens across culture types.

### Primer Preparation and Testing:

In various embodiments, candidate isothermal amplification primers were generated from the review of literature and through NCBI BLAST analysis of potential candidate gene regions. LAMP primers were designed using PrimerExplorer V4 software based on consensus sequences obtained for target genes. Sensitivity, specificity and reproducibility for LAMP primers were first evaluated using purified genomic DNA isolated from known cultured strains of target pathogenic microbes with known hospital antibiotic sensitivity and resistance results. The strongest performing primer sets for each target gene were evaluated against unprocessed duplicate clinical culture samples on 96-well plate format using real-time PCR detection by Roche LightCycler 96 System analysis and visual discrimination of color change of reactions in the presence of Eriochrome Black Dye (EBT) after incubation on a 96-well plate-adapted heat block.

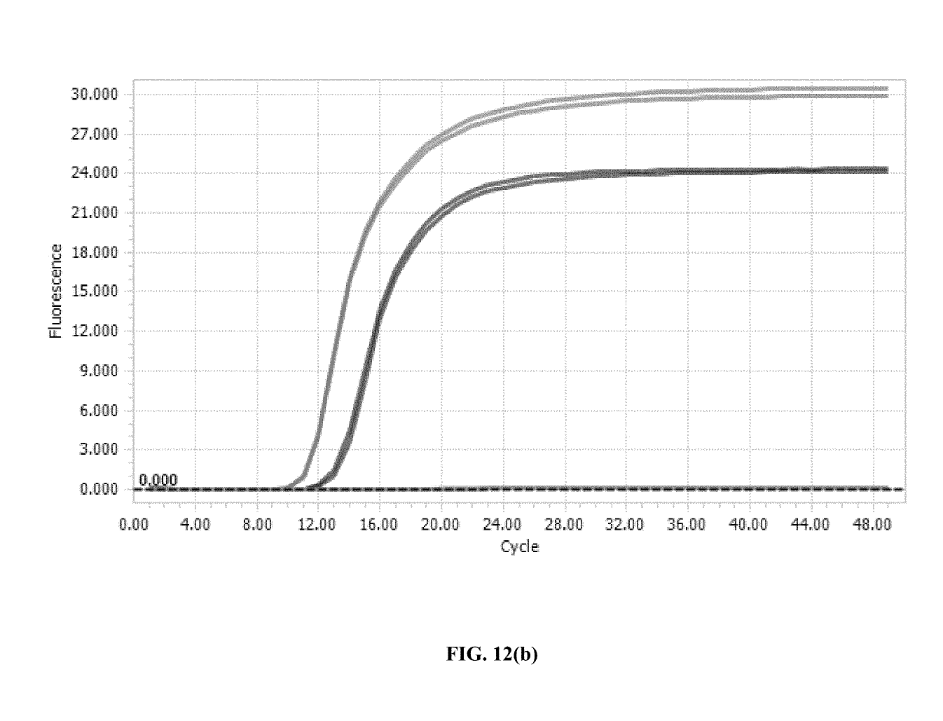

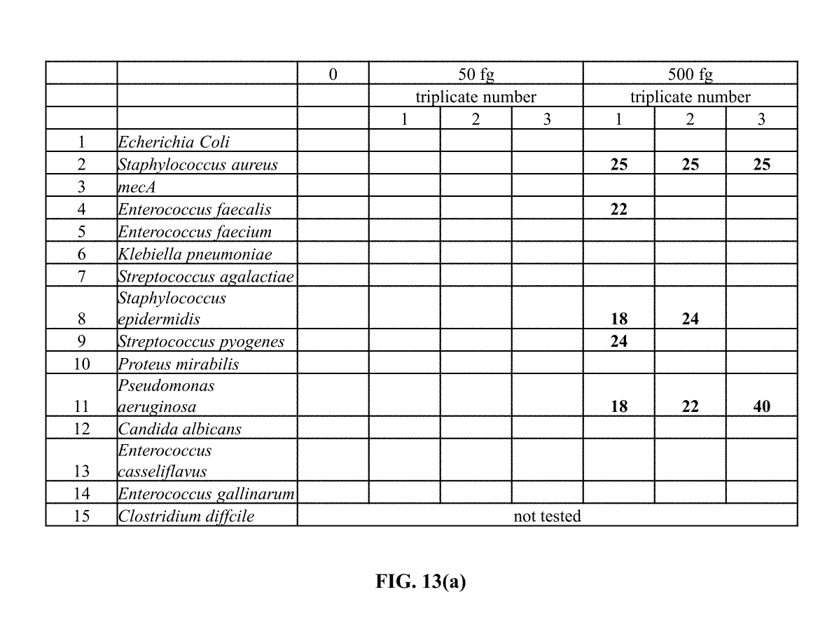

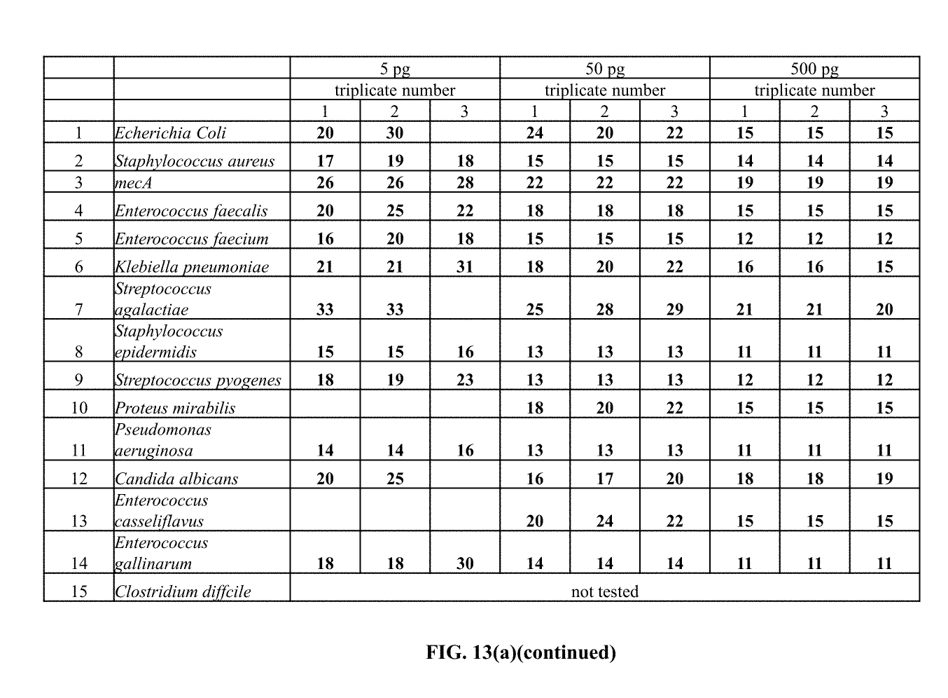

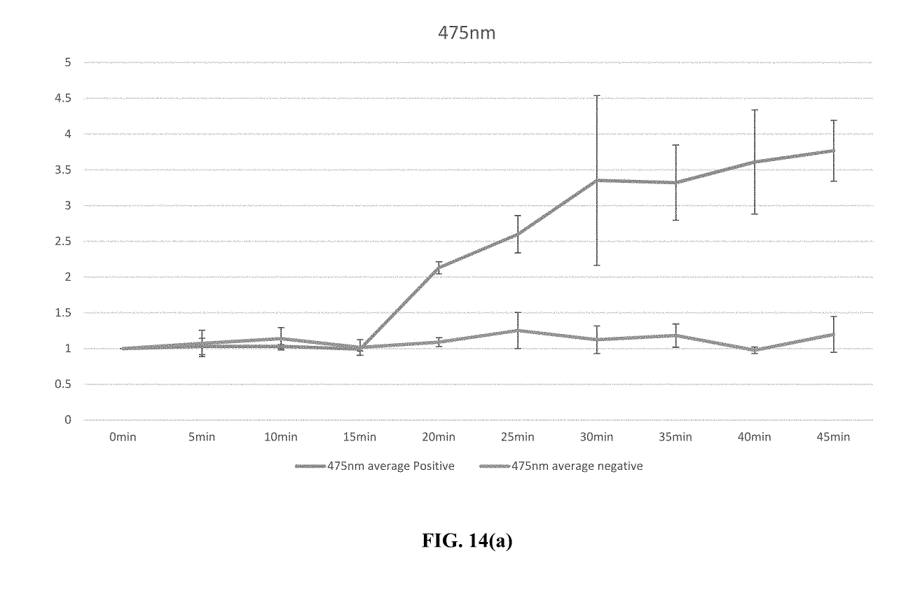

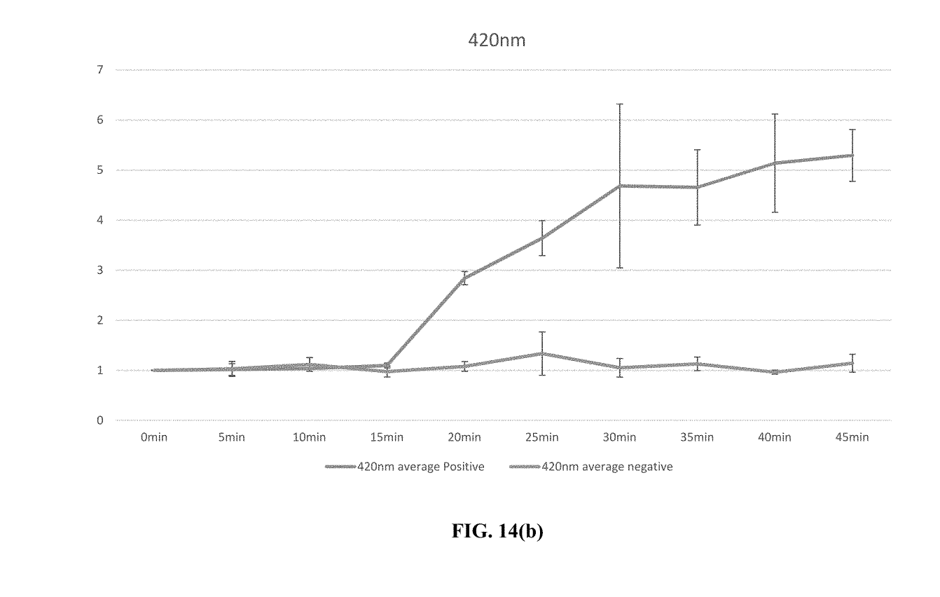

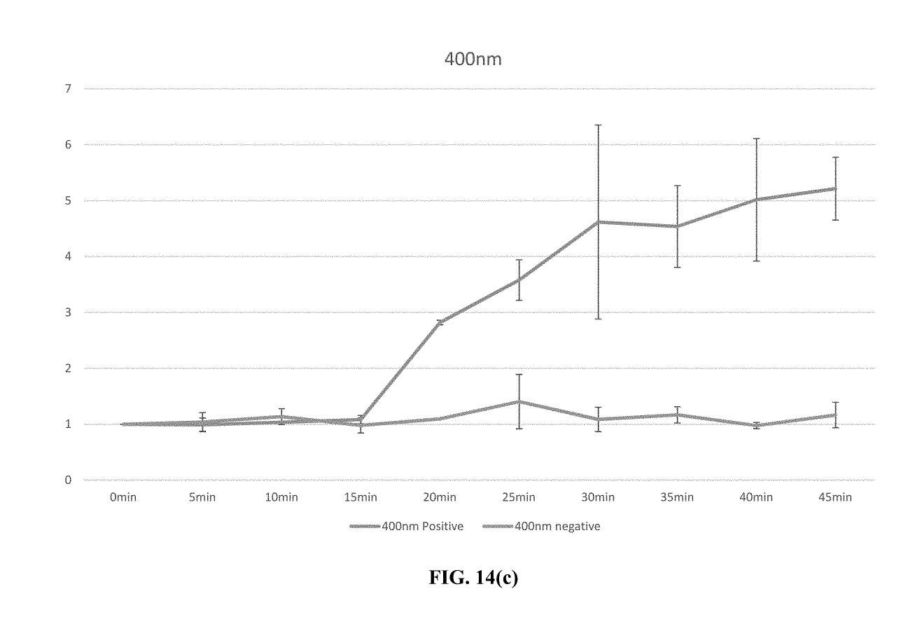

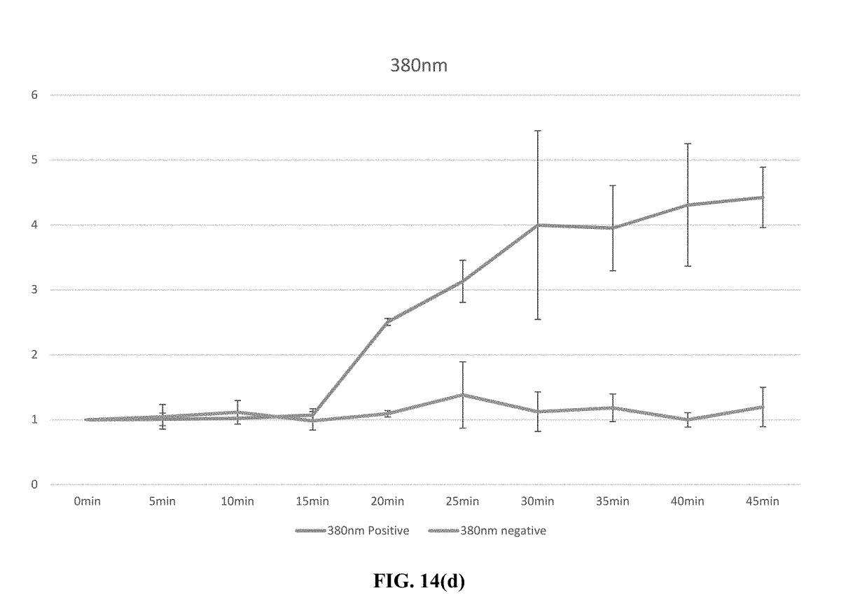

In one embodiment, quantification of purified genomic DNA from cultured hospital and ATCC sources for the methods 300, 400, and 500 was performed through standard curve generation derived using LightCycler and EBT-based color change analysis (e.g. as shown in FIGS. 12(a) and 12(b)). Spiked purified DNA in urine and blood was analyzed with a LightCycler instrument to estimate primer amplification stability in human derived samples. Spiked whole cultured pathogen cells (except for C. difficile) were diluted into urine. Only E. coli was spiked into blood, concentrated using EconoSpin, and quantified using LAMP analysis (as shown in FIG. 11). Previous studies investigated whole spiked pathogens into blood for LAMP analysis using an alternate platform. Primers showing positive amplification from non-target DNA within 50 min were excluded from the subsequent tests. Detection thresholds for primer sets using purified DNA ranged from 5 pg at 27 min (mecA) to 50 pg at 20 min (P. mirabilis) by the LightCycler analysis (as shown in FIG. 13). In another embodiment, isolates were positive from 5 pg to 500 pg at 35 min reaction time with EBT-LAMP analysis (FIG. 14). Color change was found for the mecA primers by EBT-LAMP analysis at 500 pg concentration of purified DNA at 35 minutes of incubation time. No EBT-LAMP samples were incubated greater than 35 minutes to avoid nonspecific primer amplification.

In another embodiment, a six-primer system was employed for the LAMP reaction detection of clinical pathogens. The primer targets were selected from literature or designed using PrimerExplorer V4 online software. In one embodiment, between two and nine primer sets, including a Forward 3 (F3), Backward 3 (B3), Forward Inner Primer (FIP), Backward Inner Primer (BIP), Loop Forward (LF) and Loop Backward (LB) for a total of six primers per target, were developed for each strain and optimized for sensitivity and specificity. Each primer set was tested with purified genomic DNA to achieve detection of >5 pg within 30 min. The most sensitive and specific primer set for each microbial target was selected for clinical sample detection (as shown in FIG. 11). For generating standard curves, genomic copies per reaction for each isolate was estimated based on mass of gDNA used per reaction and the average genome size for the respective species.

In another embodiment, direct amplification of target nucleic acids utilizing isothermal PCR techniques are adapted toward direct and rapid processing of clinical samples for accurate detection of the primary pathogens responsible for clinical infection across a wide variety of clinical types at the point-of-care. Moreover, additional samples to elucidate testing limitations and generalizability among the pathogens to allow a paradigm change in clinical microbiological testing and infection surveillance and control are determined.

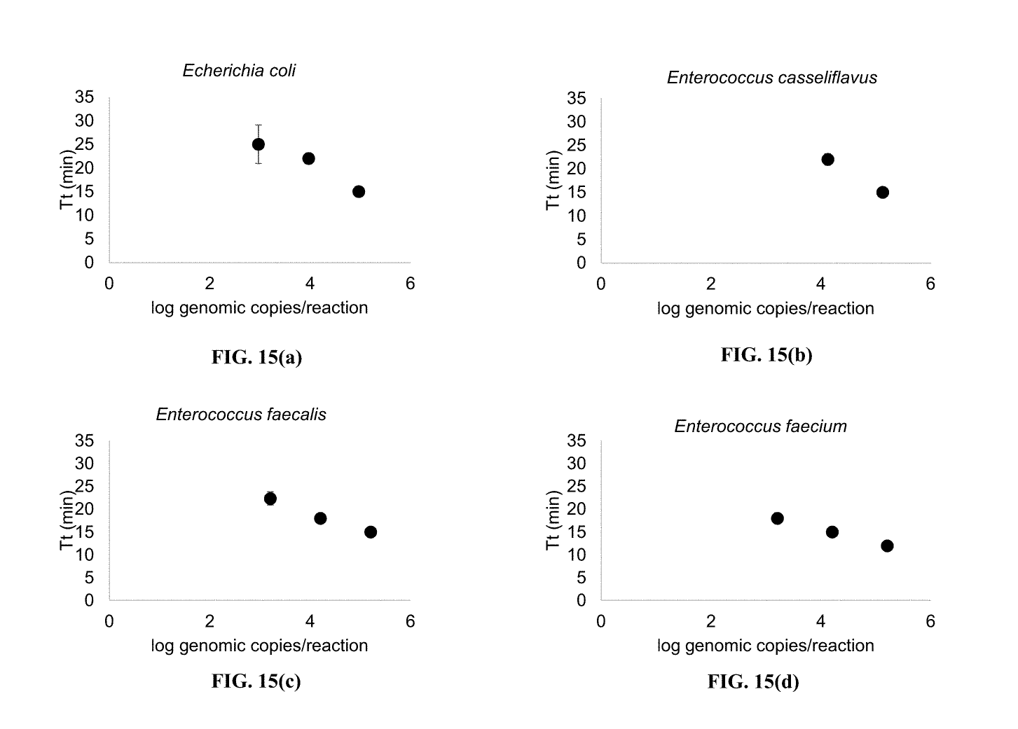

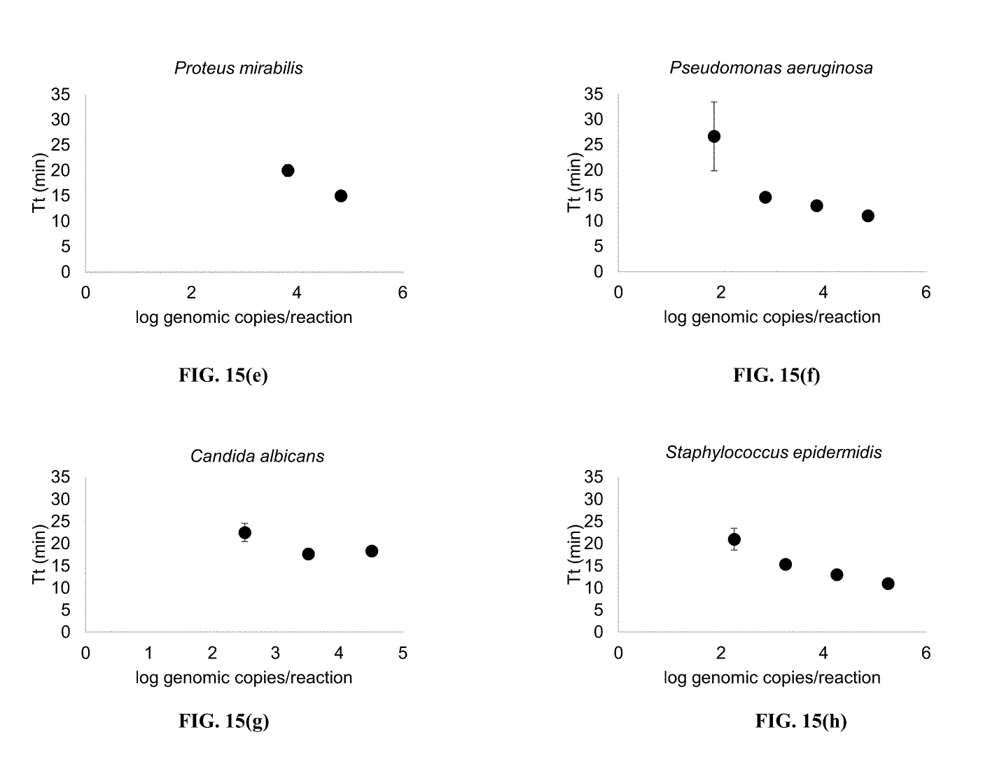

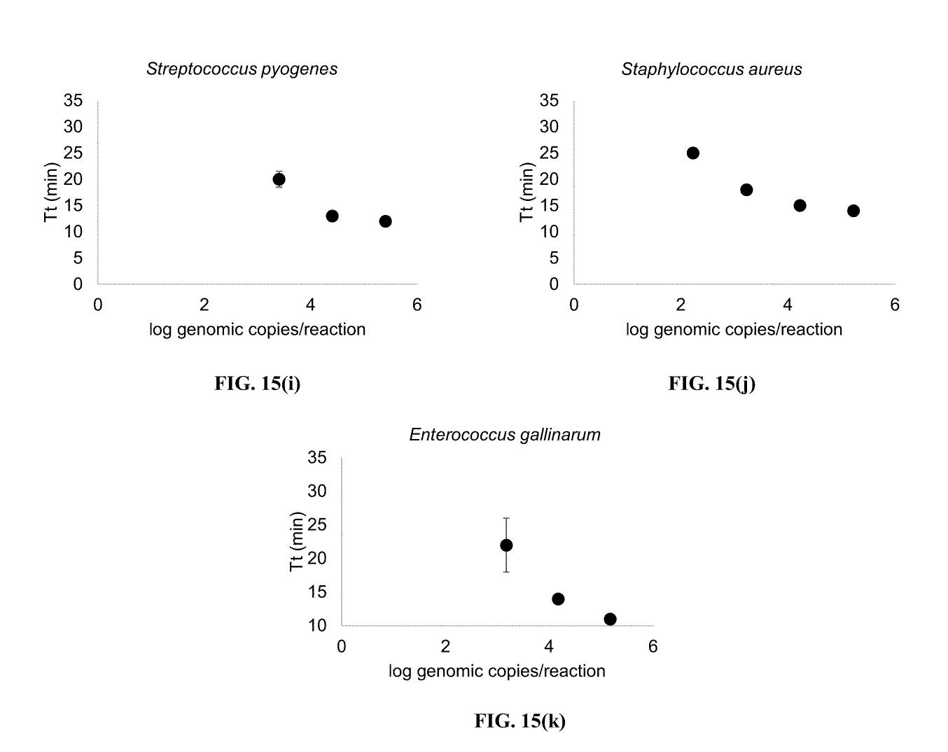

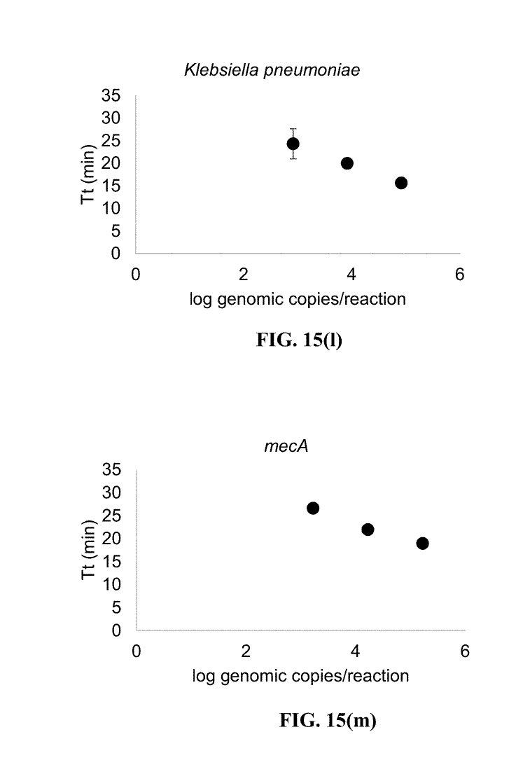

In various embodiments, the extraction of DNA from bacterial strains for initial primer testing was performed with a DNeasy Blood & Tissue Kit (Qiagen) according to the manufacturer's instructions. The extraction includes 1.5 mL of bacteria growth culture for each strain. The elution volume was 100 μL and the concentration was finally adjusted to 5 ng/μl. In another embodiment, ten-fold serial dilutions of purified genomic DNA from 500 pg/μL to 5 fg/μL for each strain were used for DNA standard curve control generation (as shown in FIG. 15(a)-(m)).

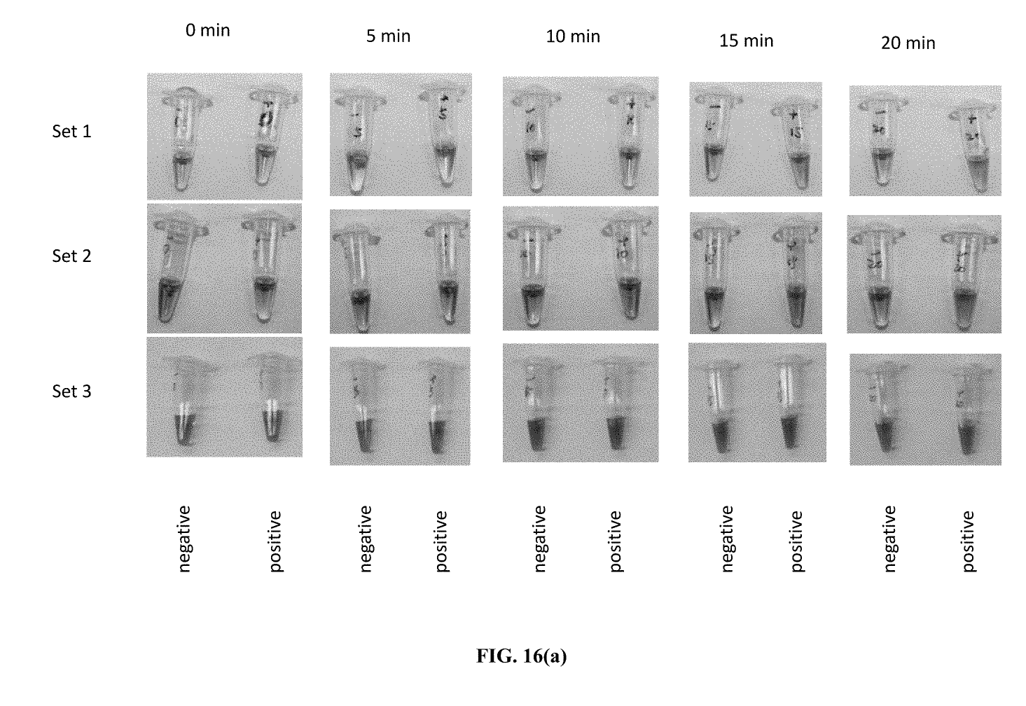

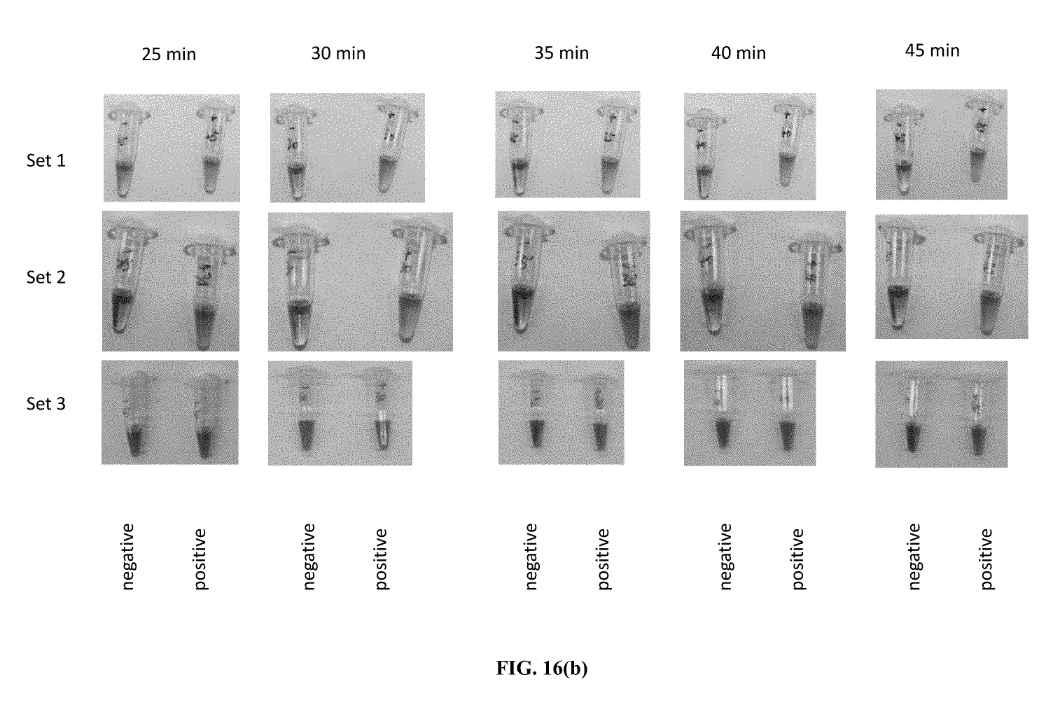

In one embodiment, the minimum concentration for detection was determined for reactions in the presence of Eriochrome Black T dye for ten-fold serial dilutions of purified genomic DNA from 500 pg/μL to 5 fg/μl for each strain with visual and spectrophotometric analysis at 5 minute intervals from 0 to 45 minutes to determine the EBT sensitivity standard curve generation (as shown in FIGS. 16(a) and 16(b)).

### Clinical Fluid Sample:

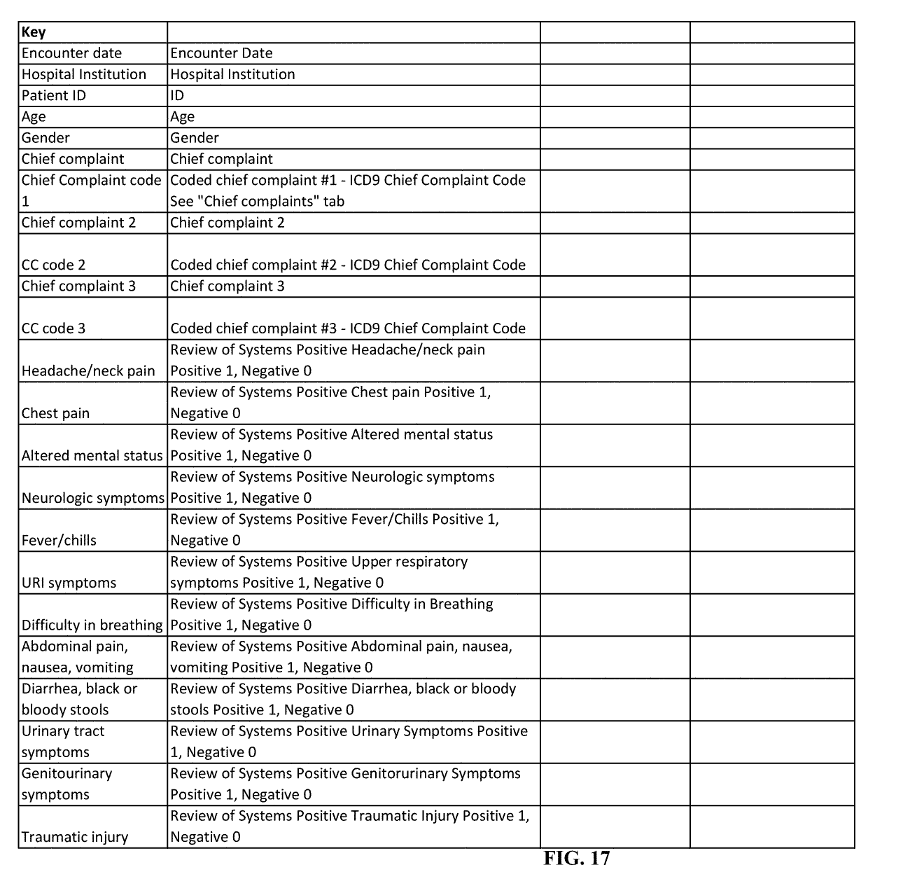

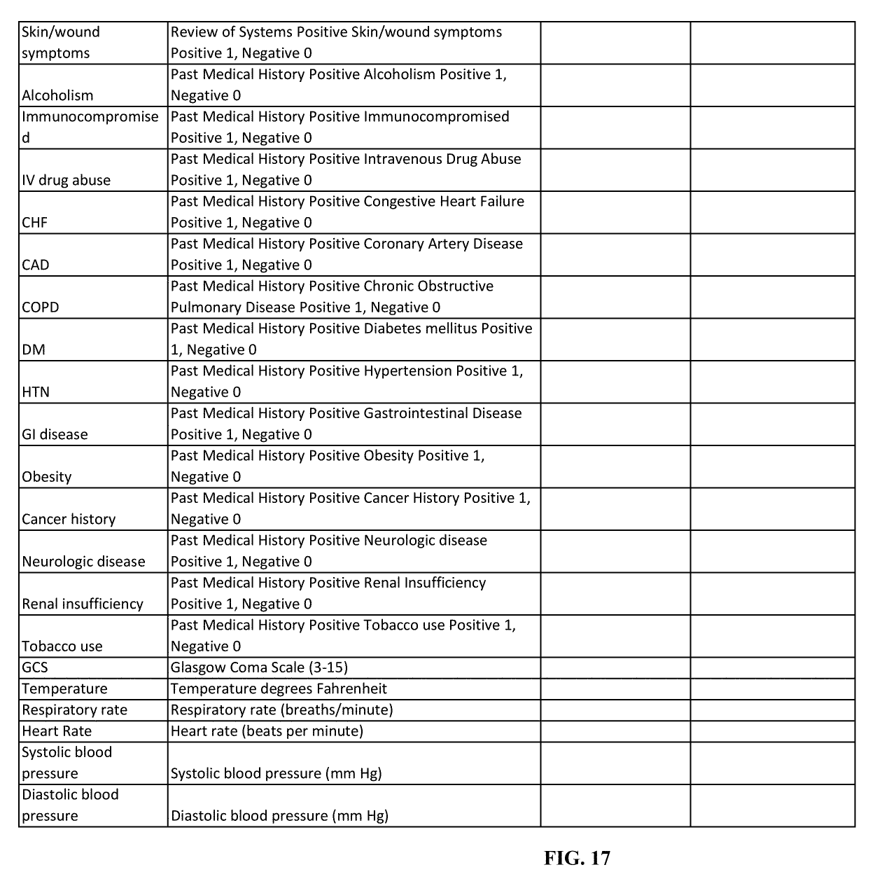

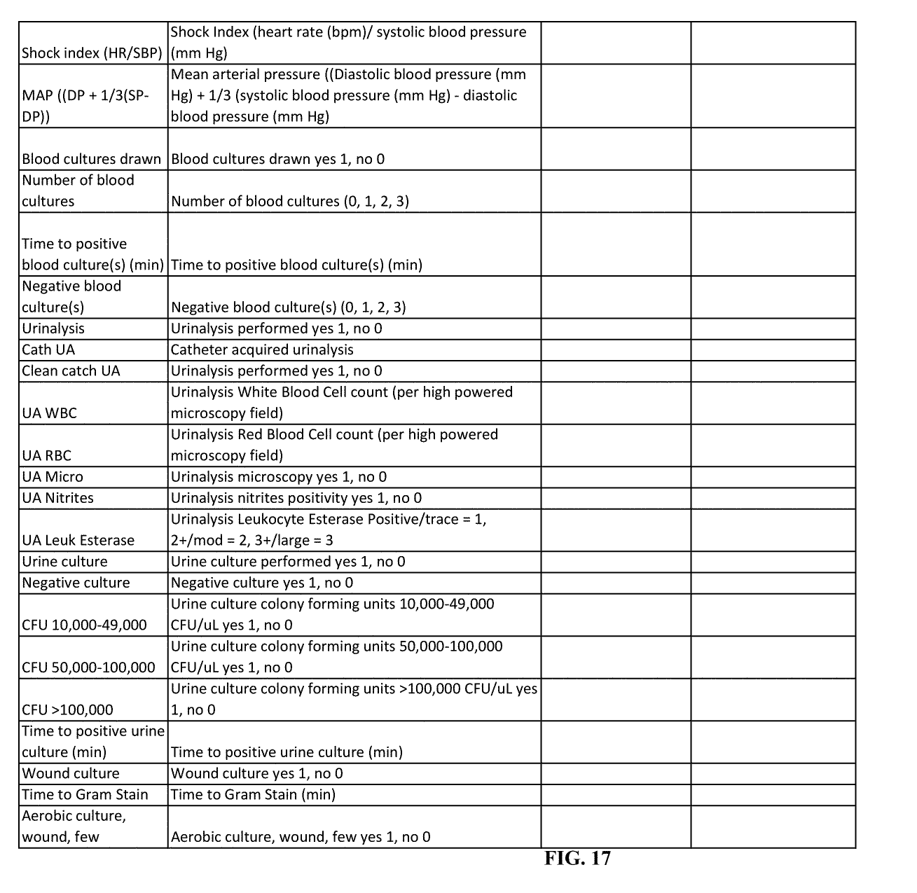

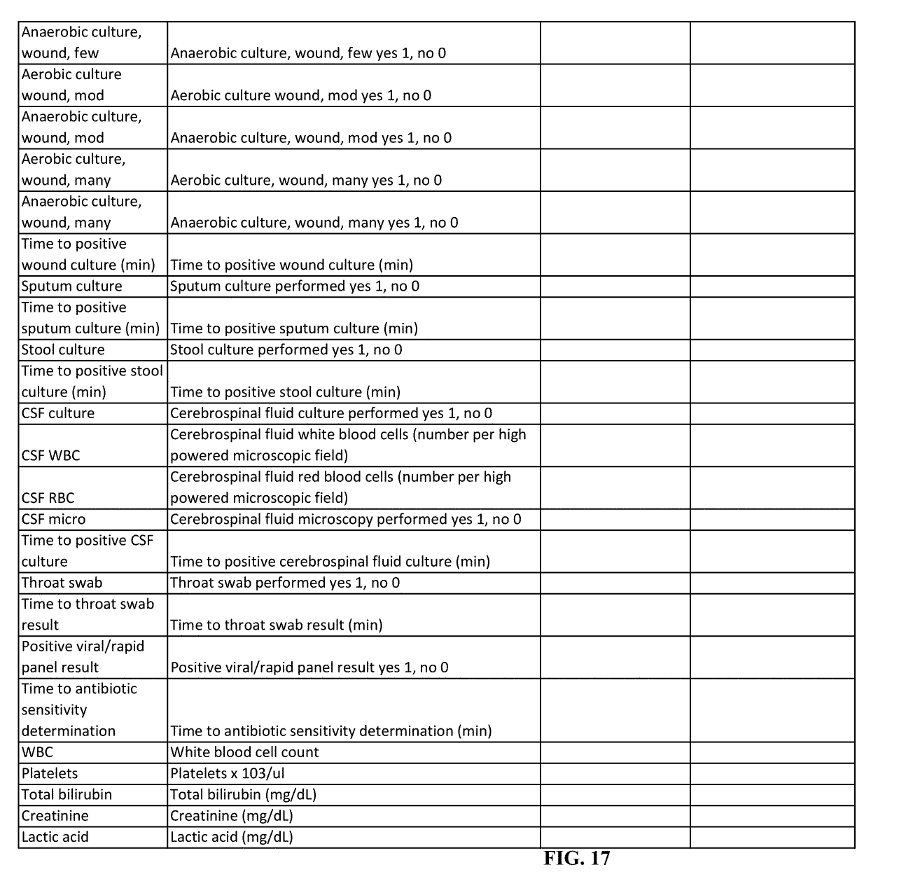

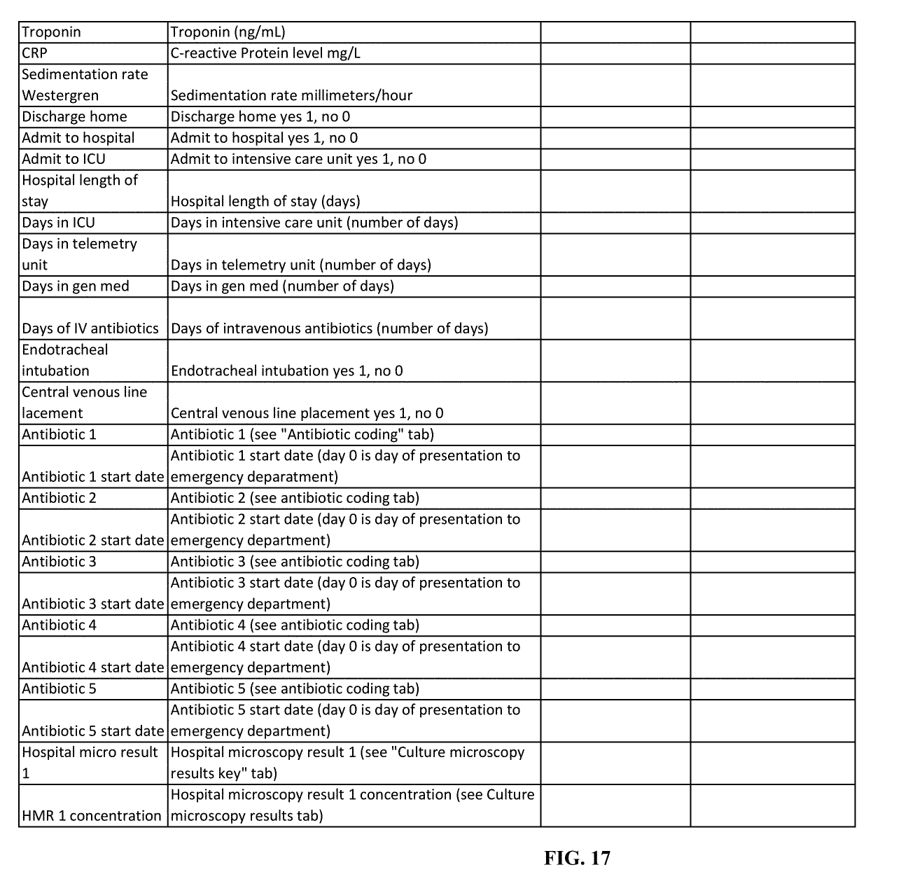

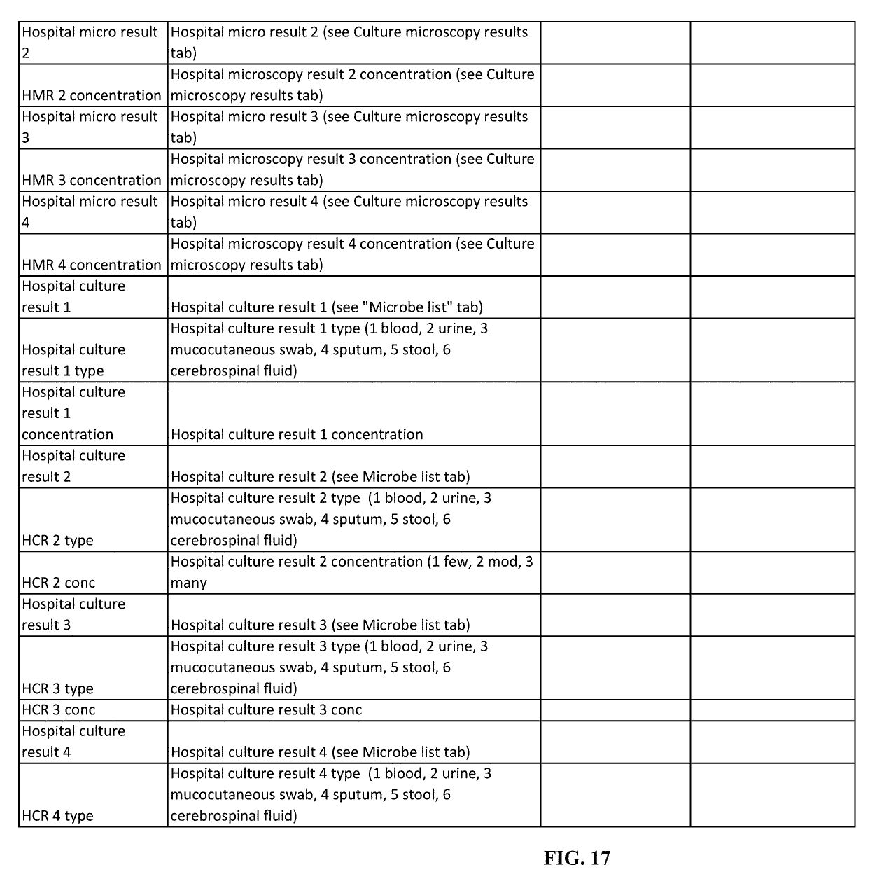

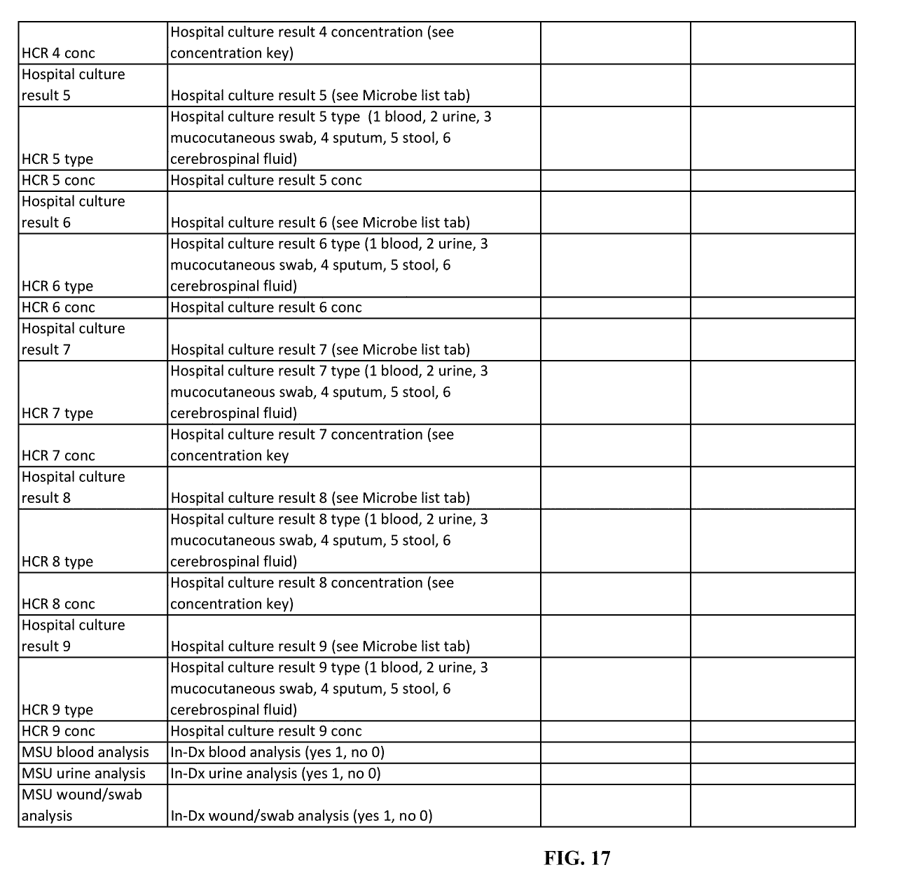

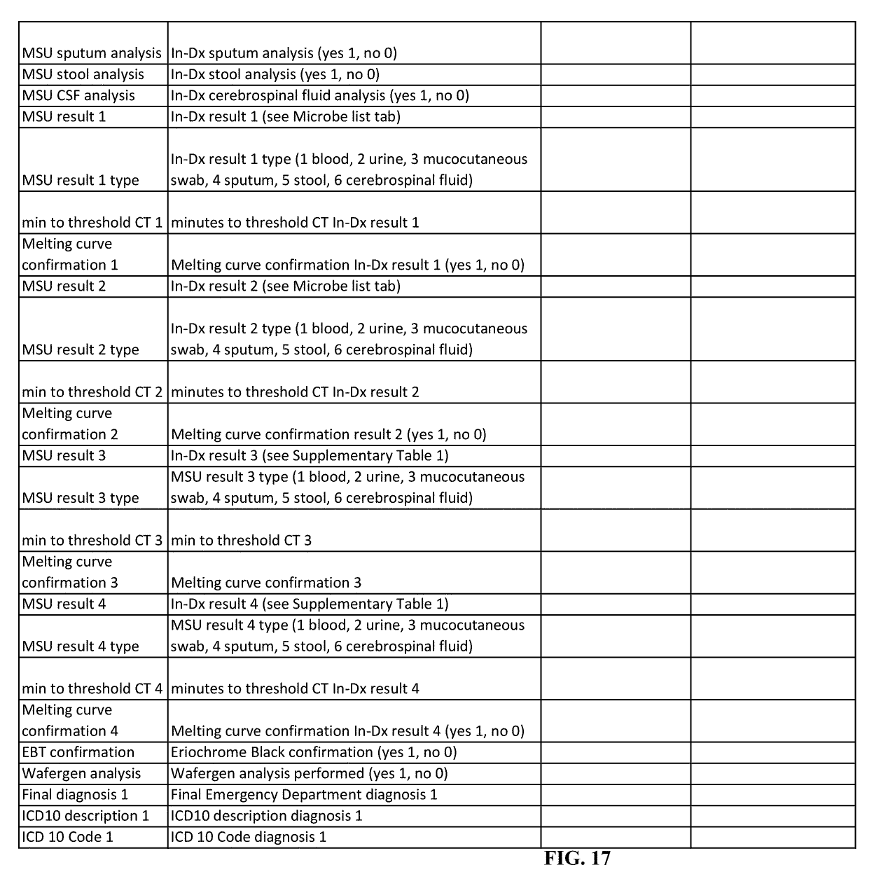

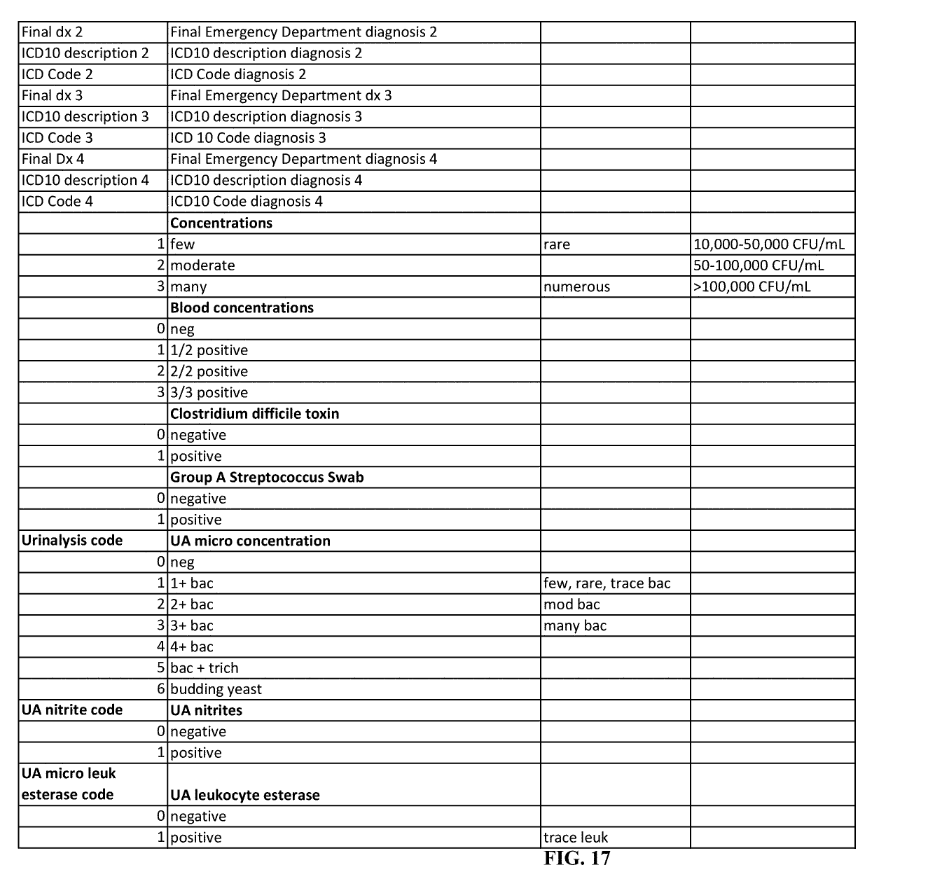

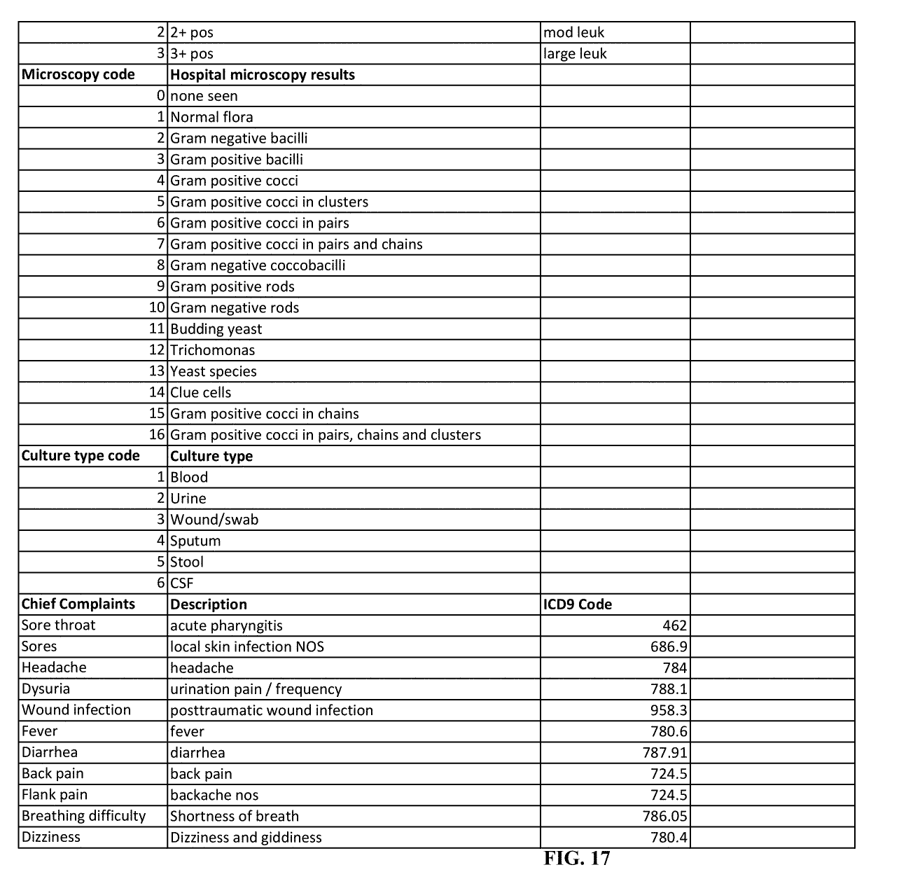

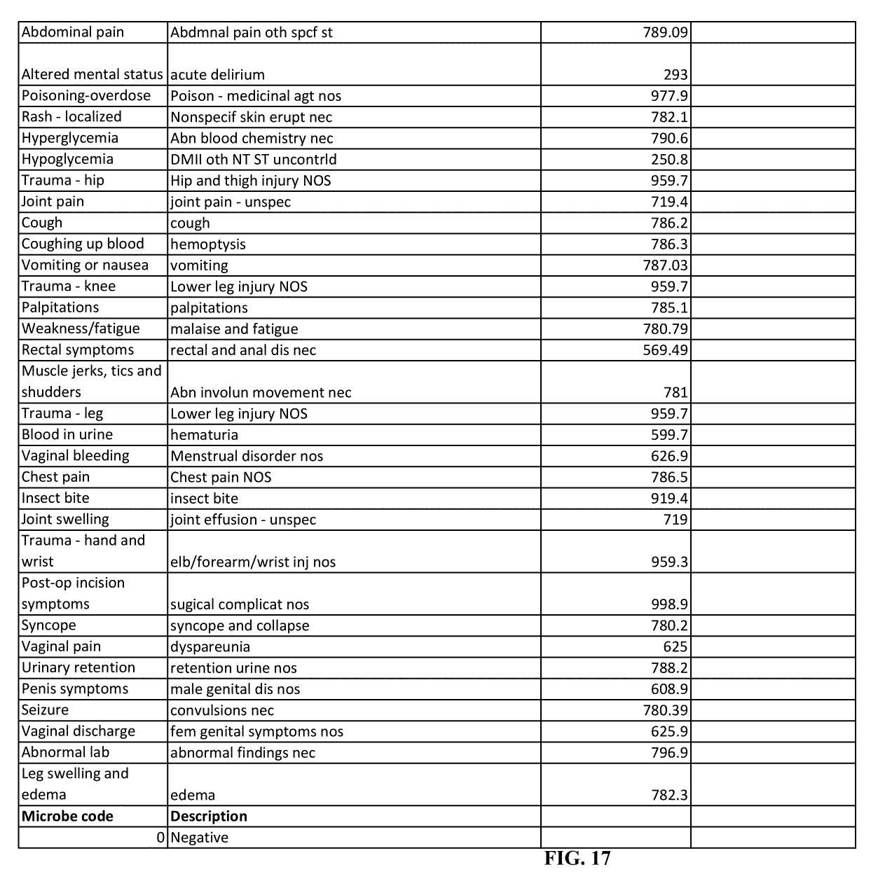

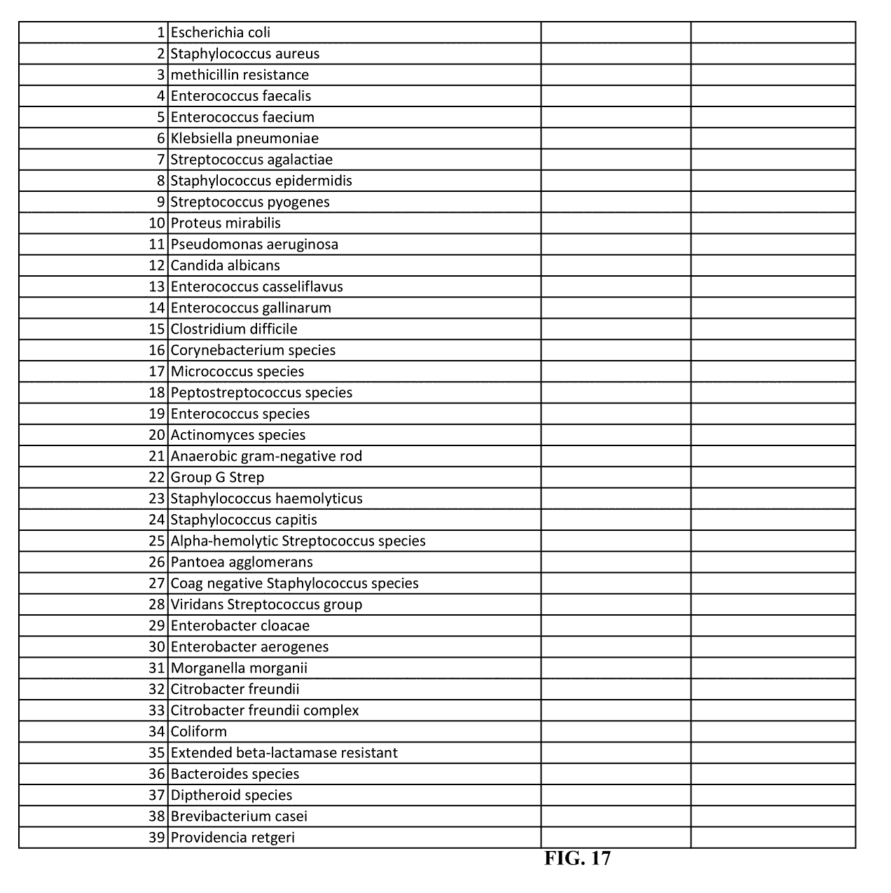

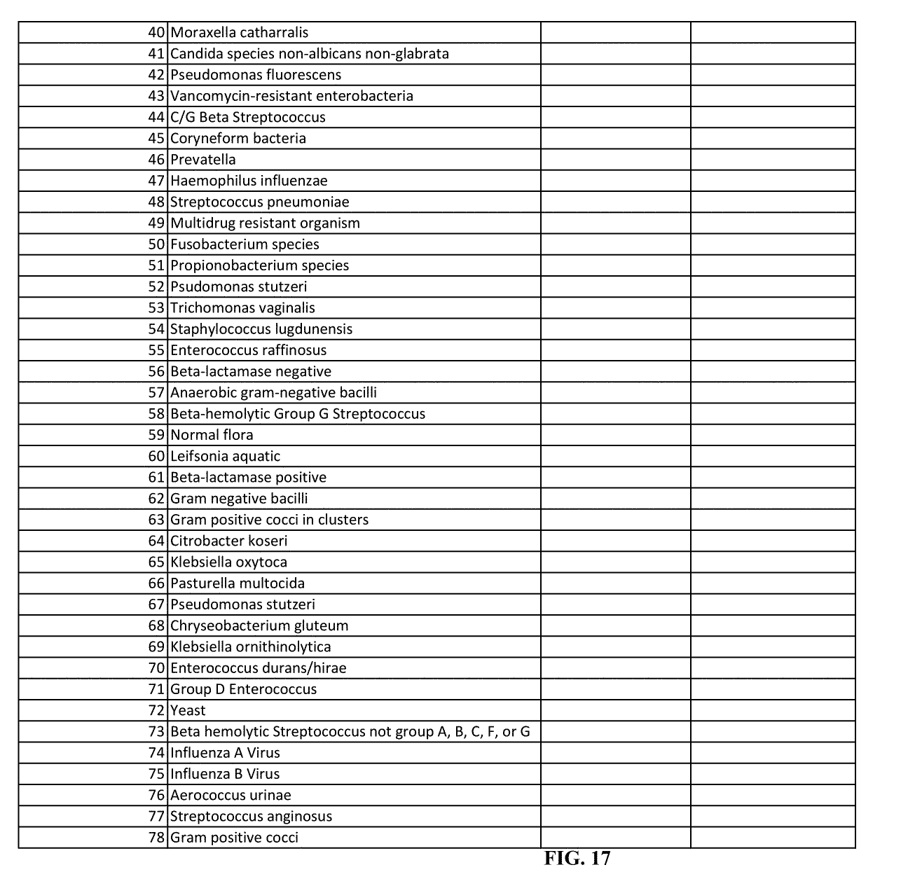

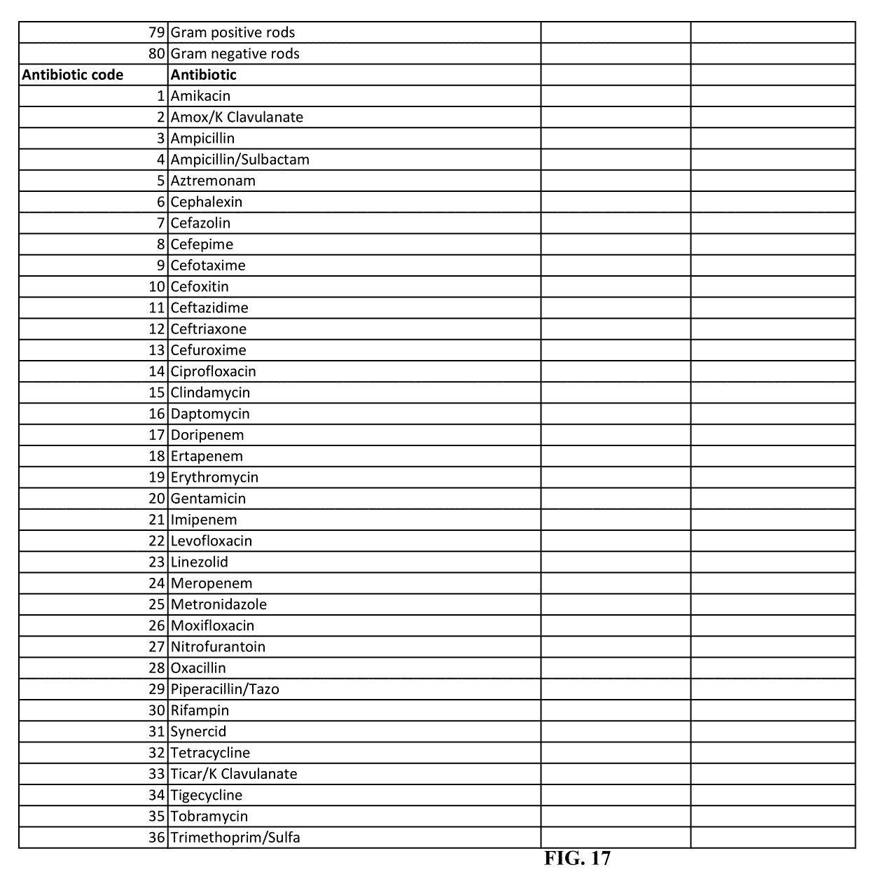

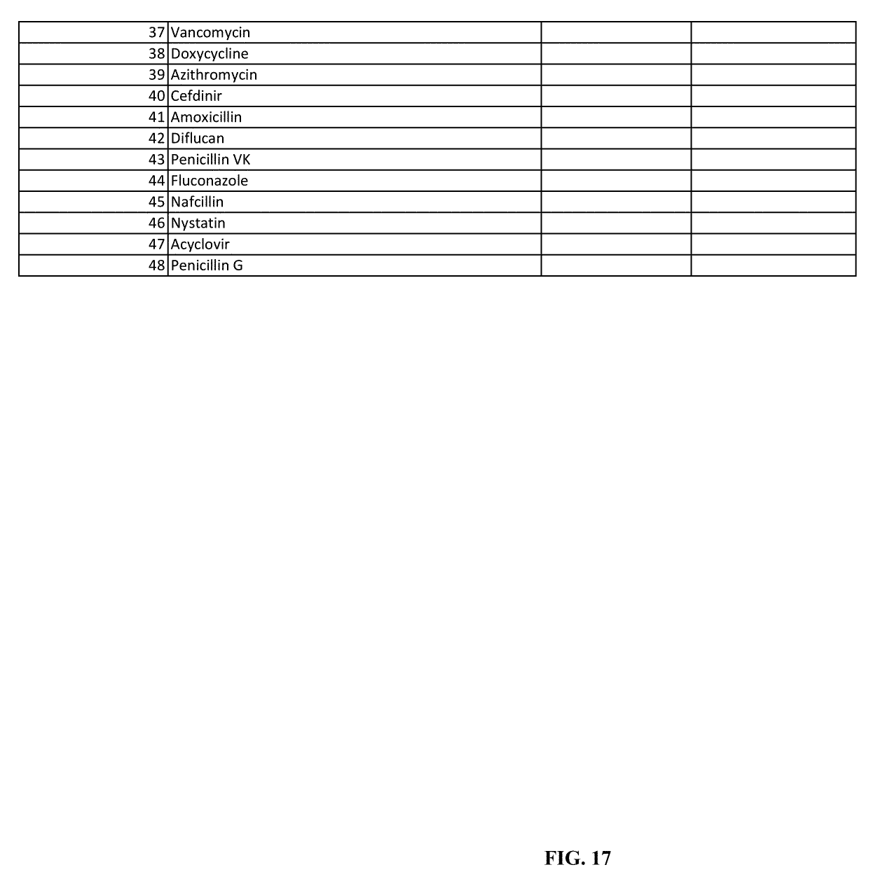

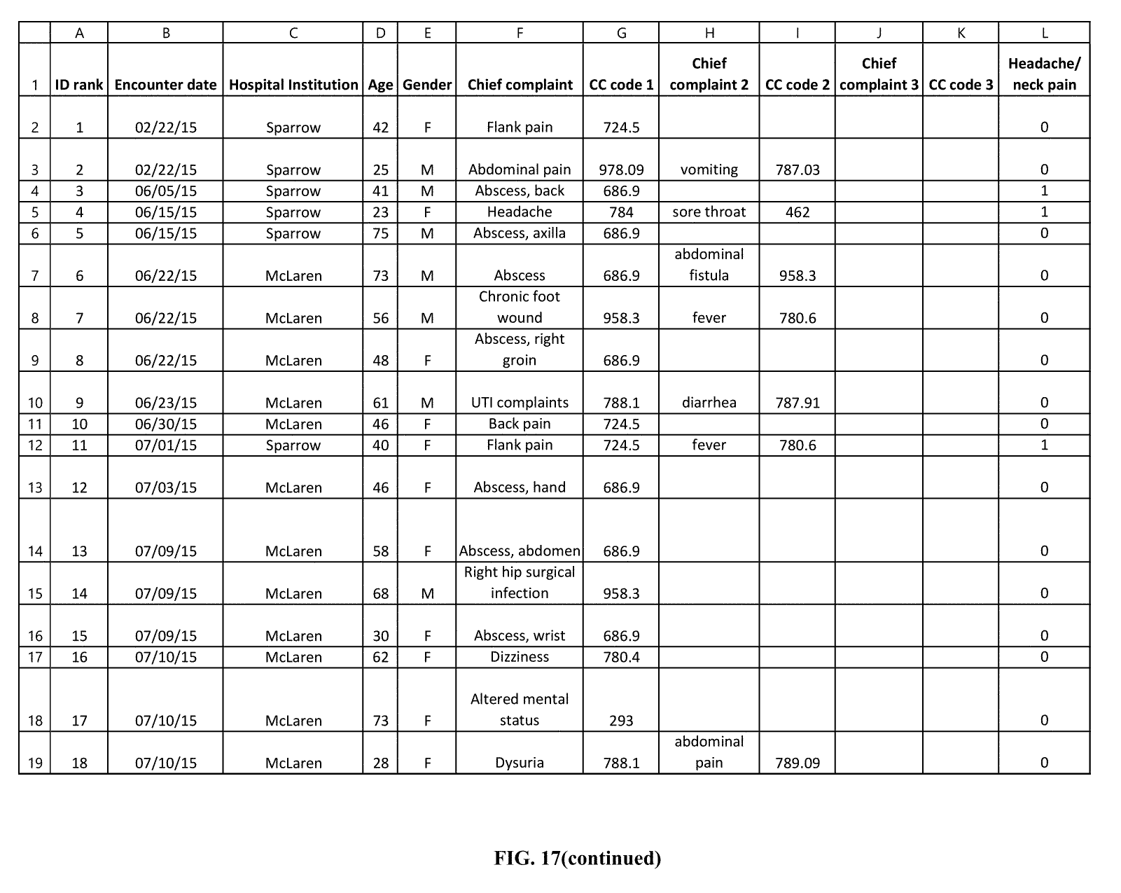

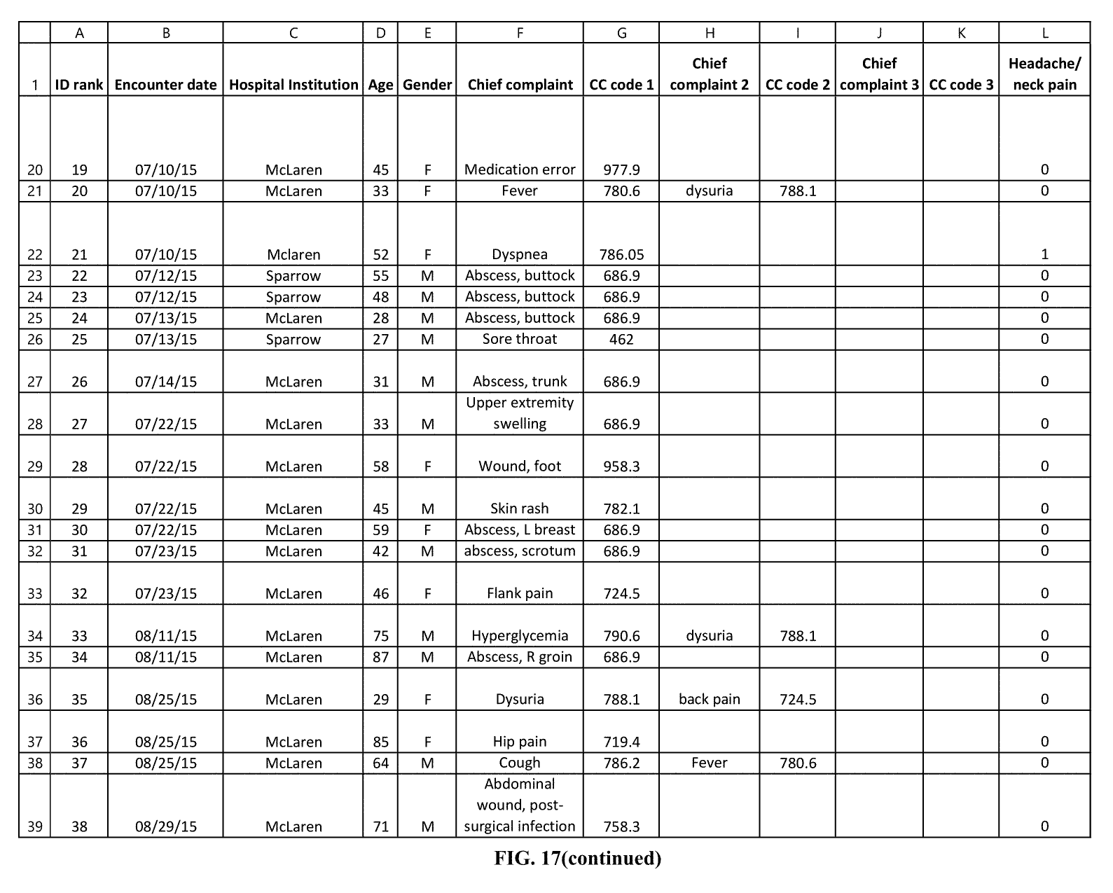

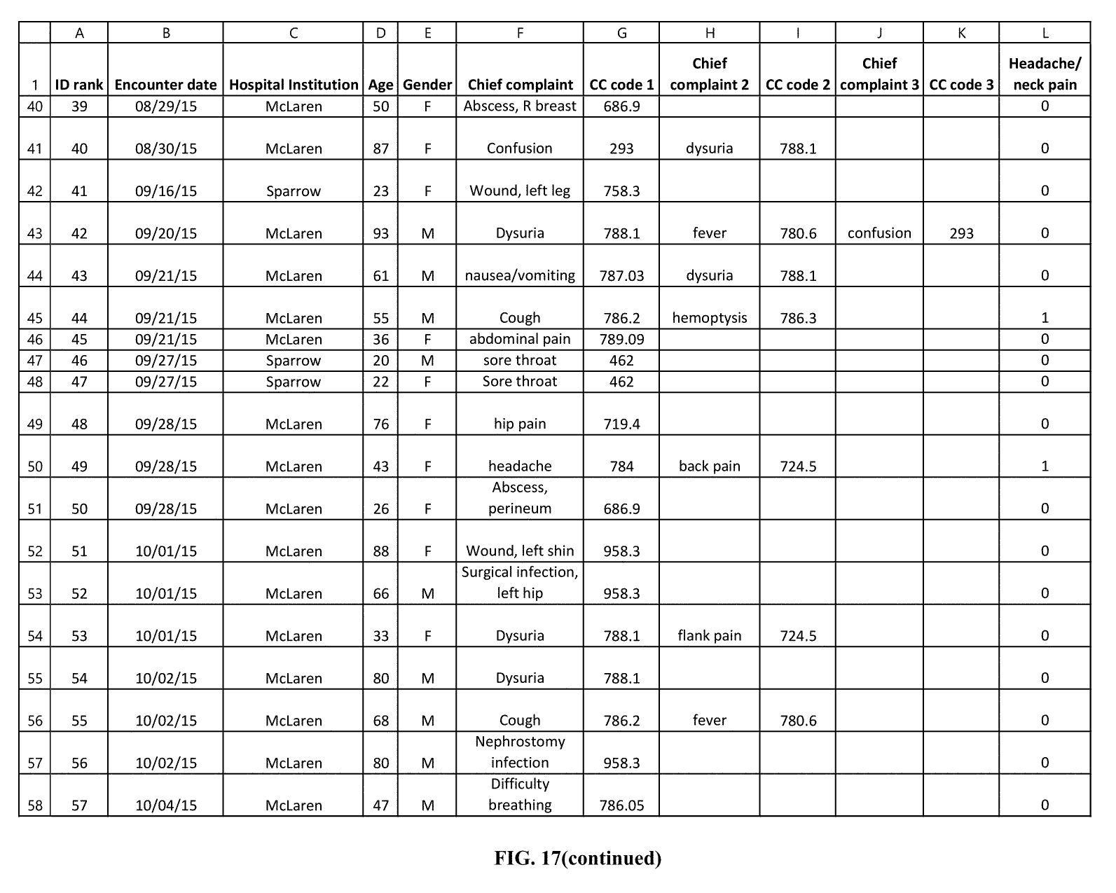

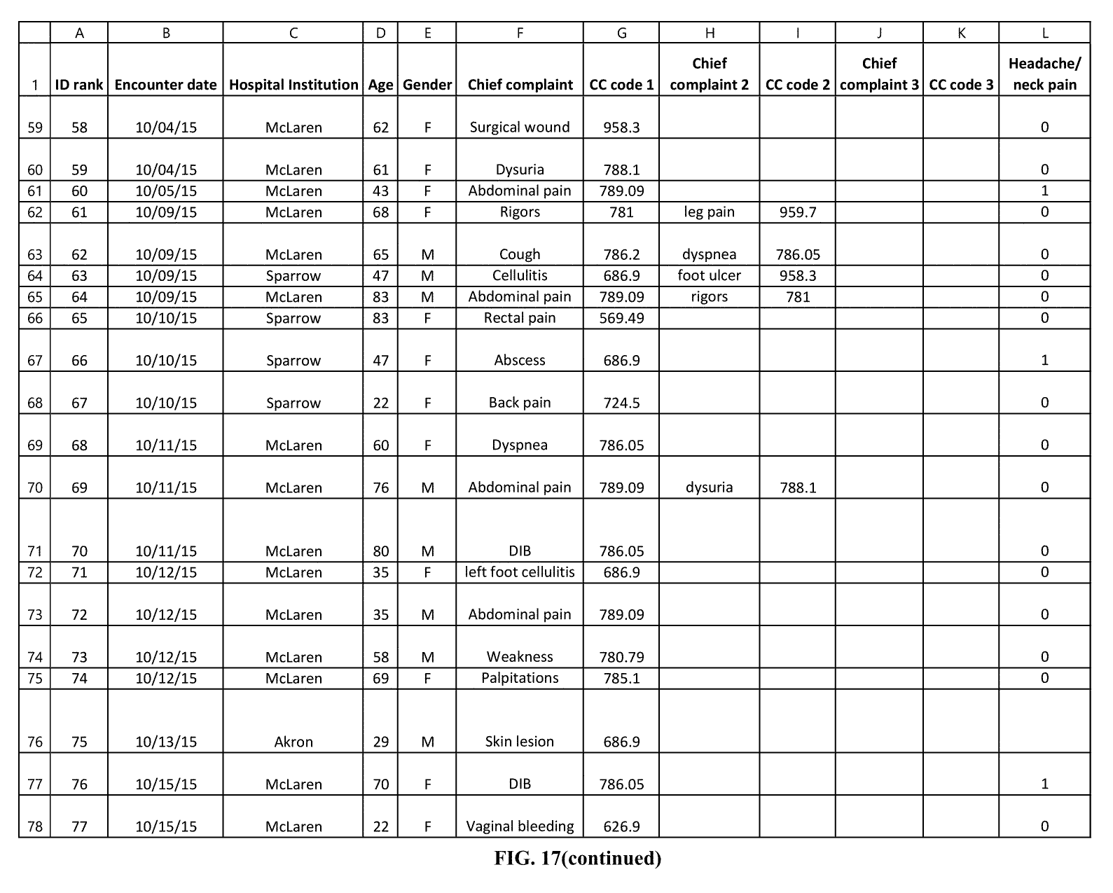

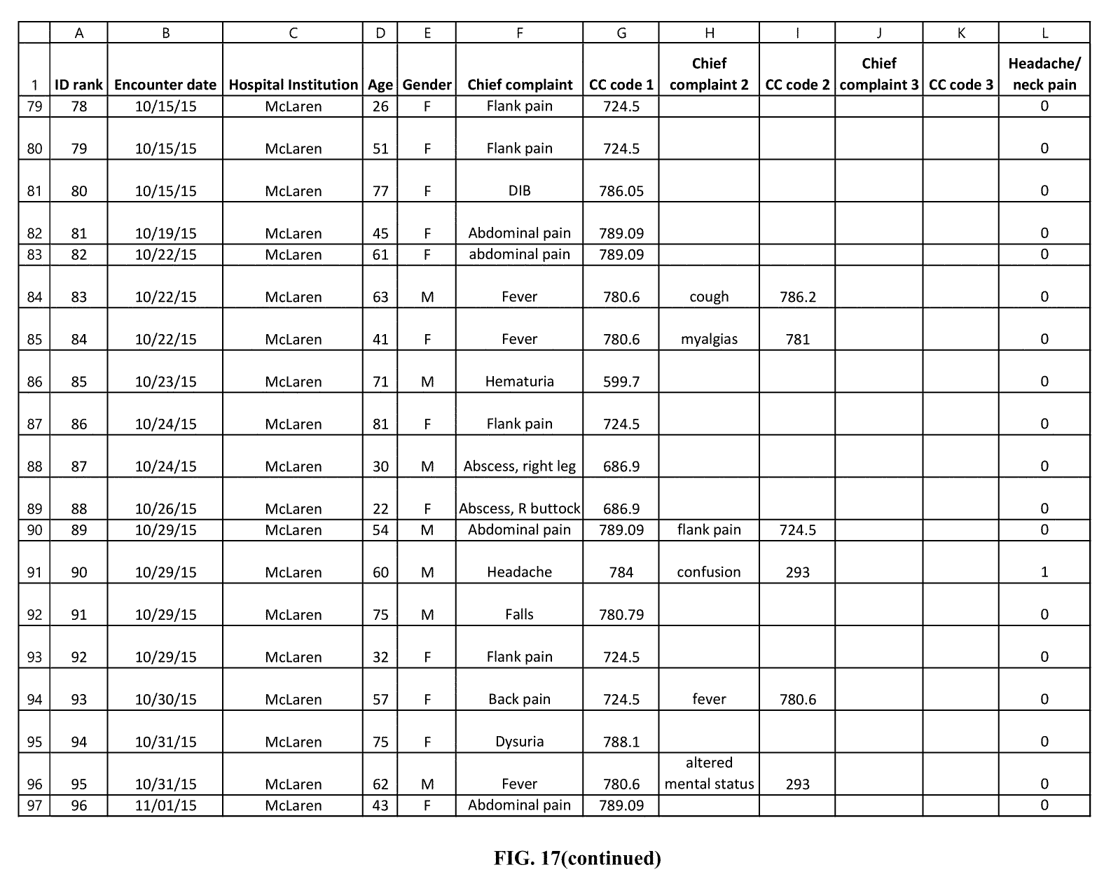

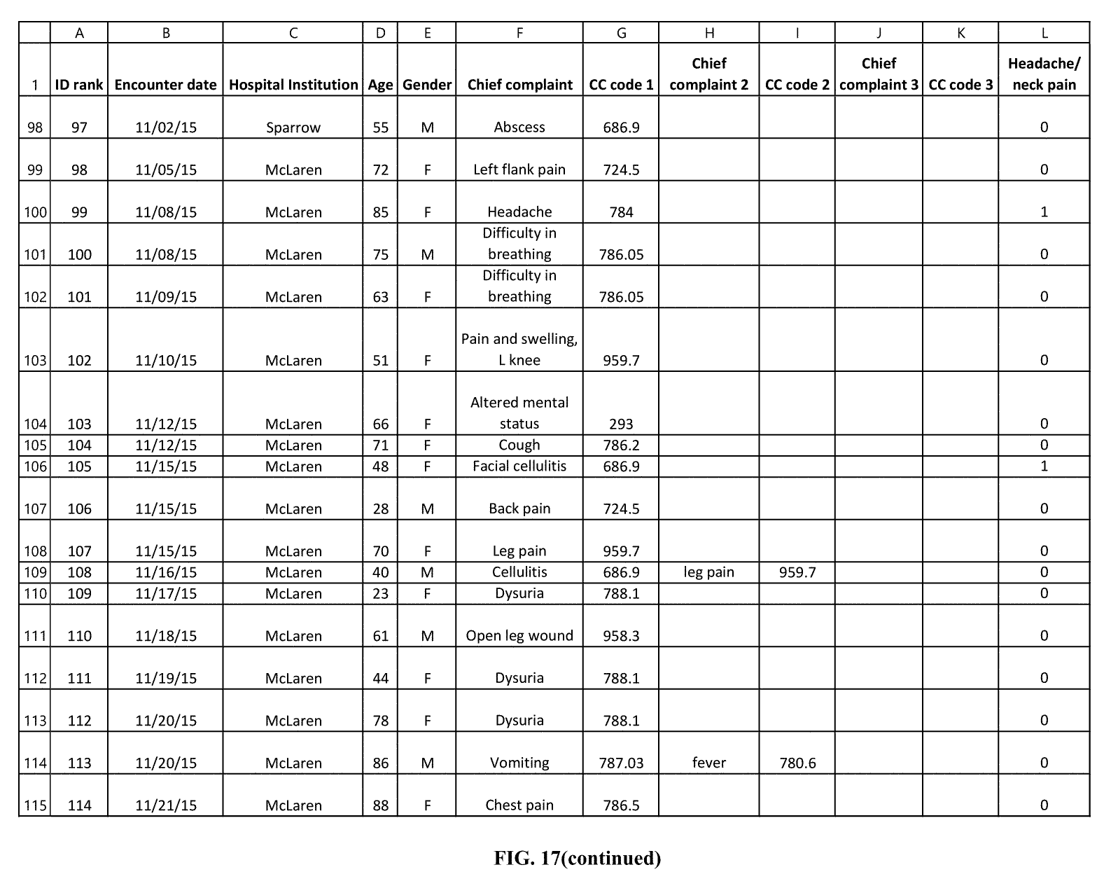

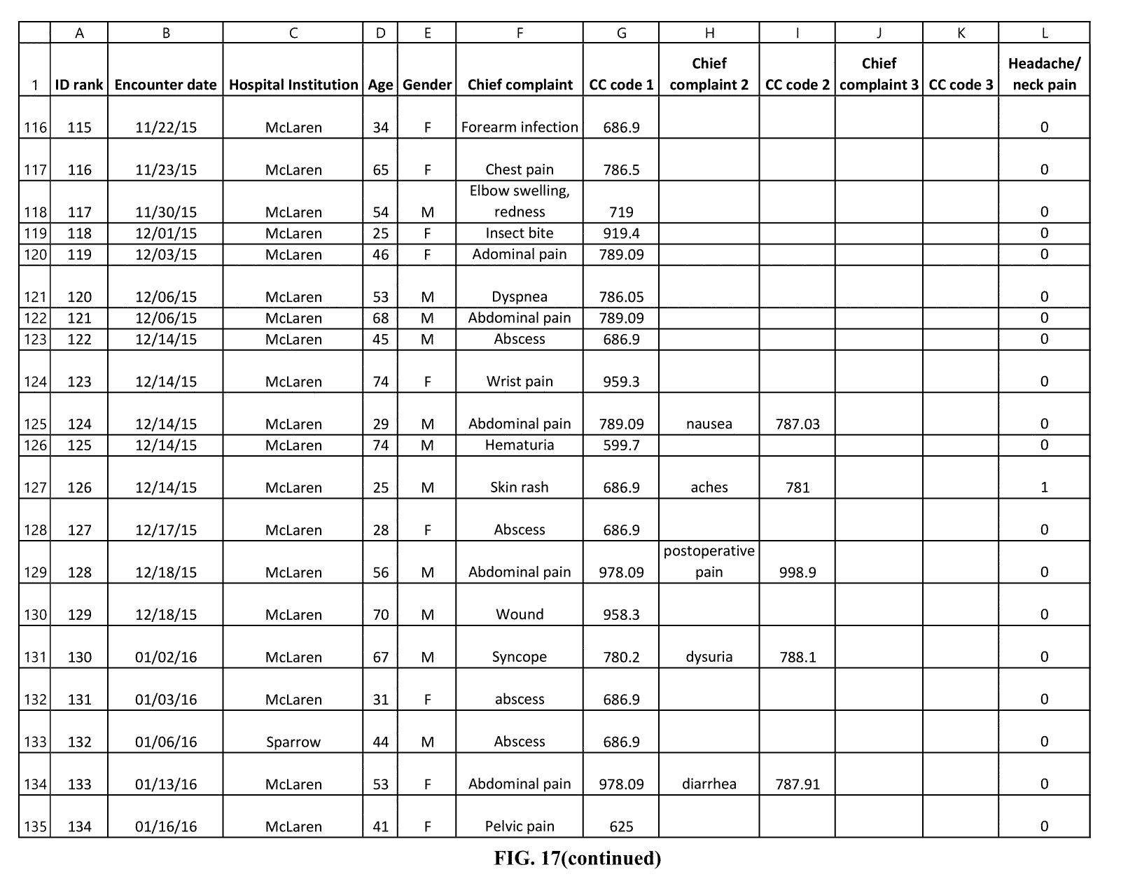

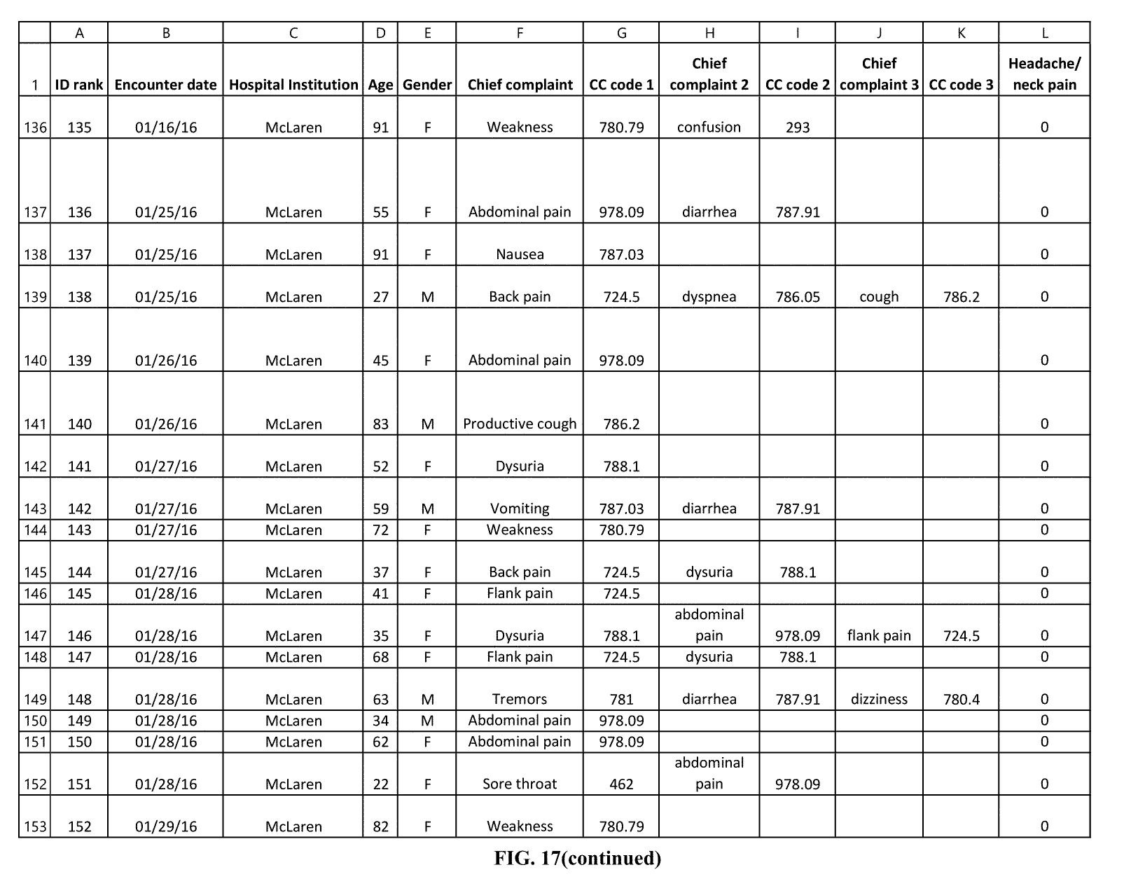

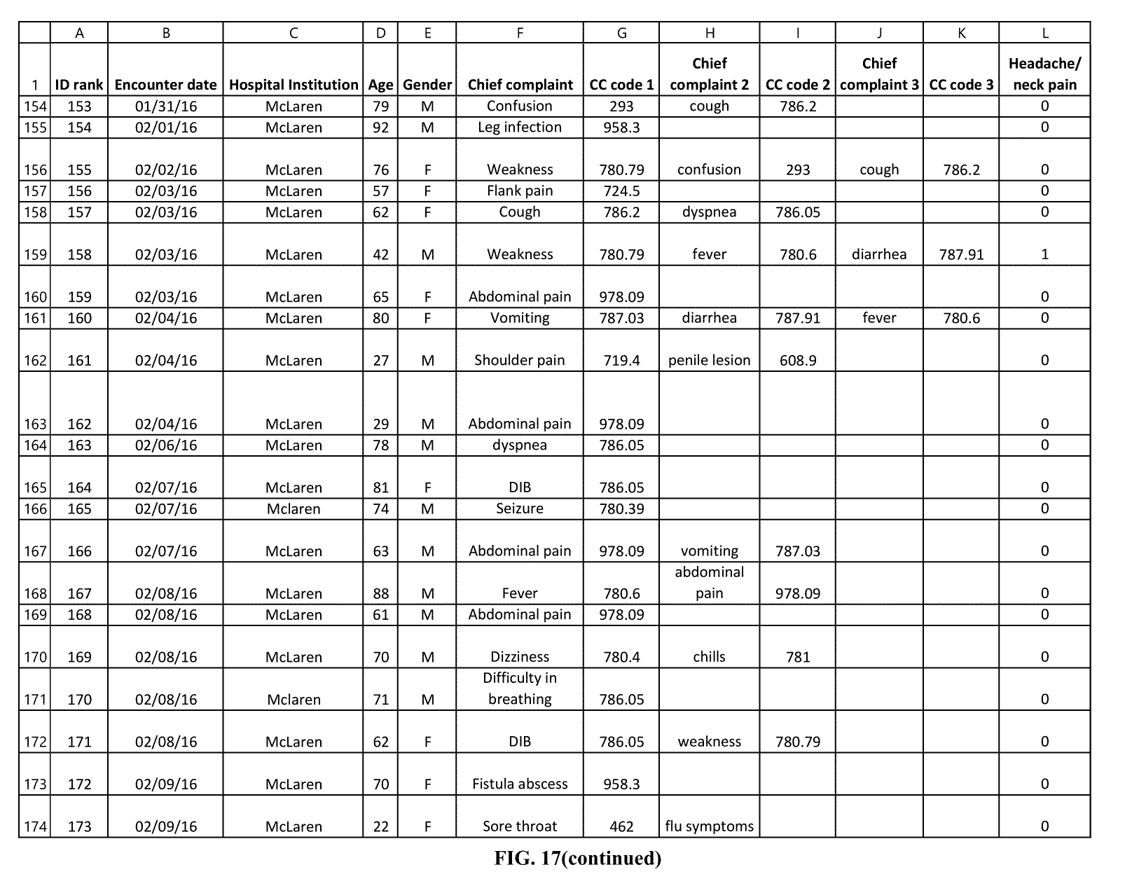

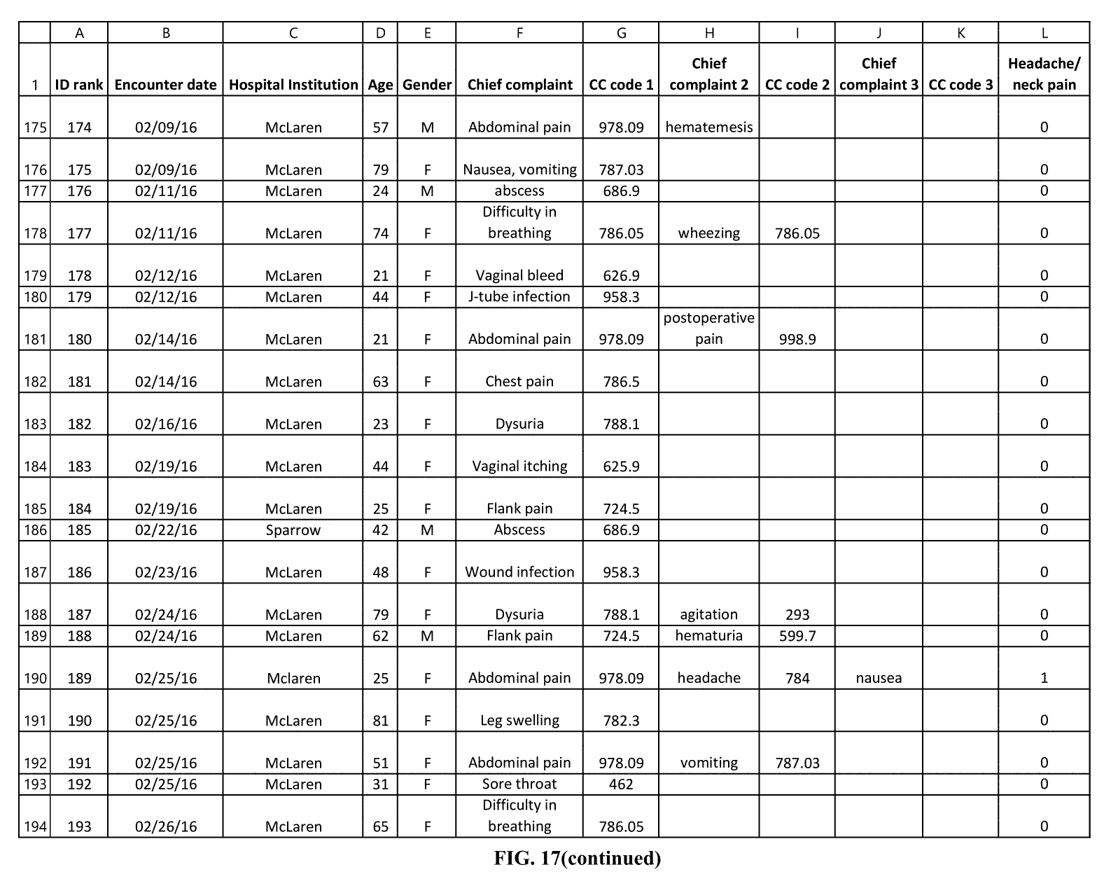

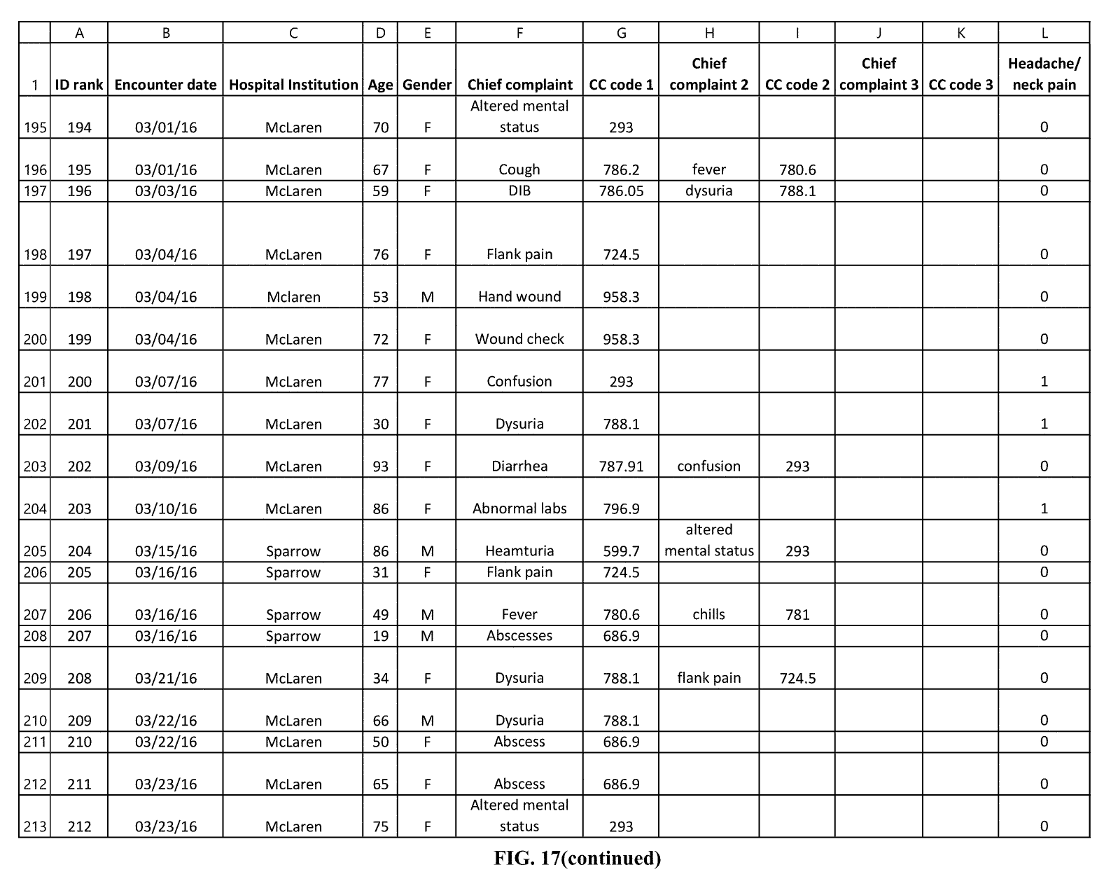

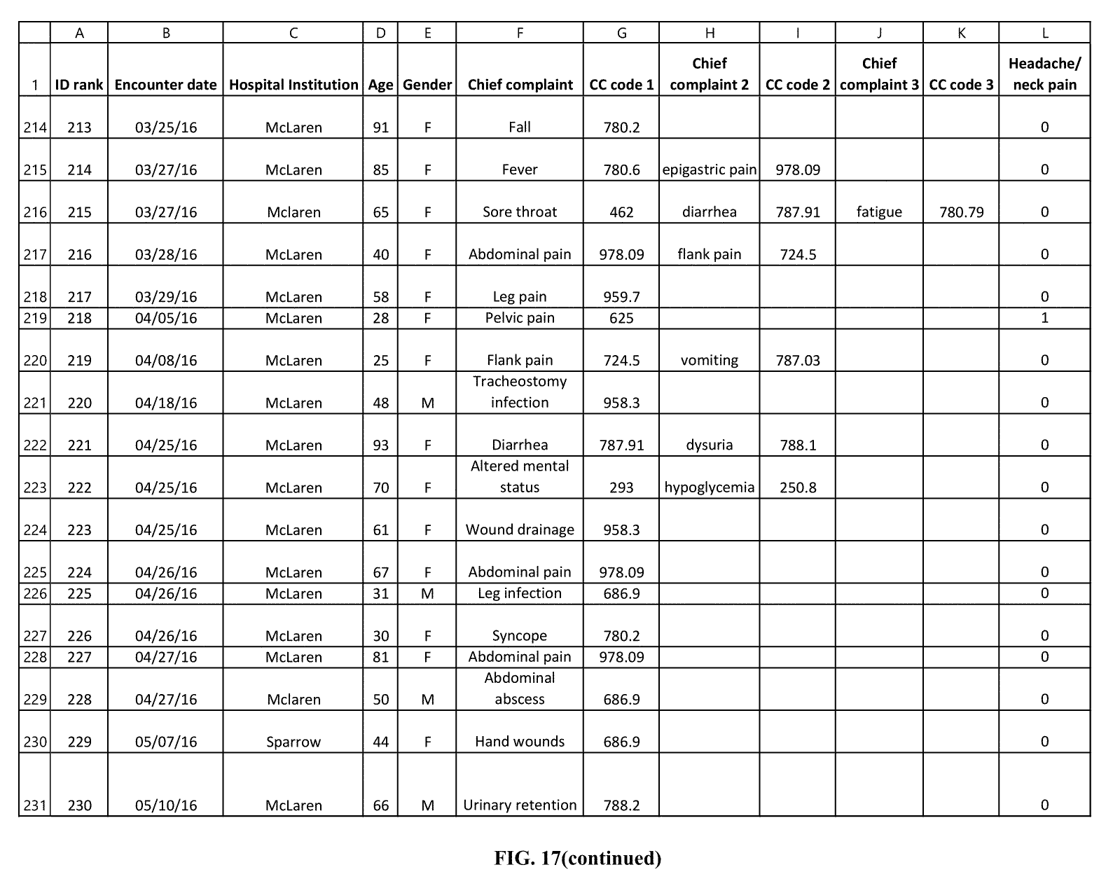

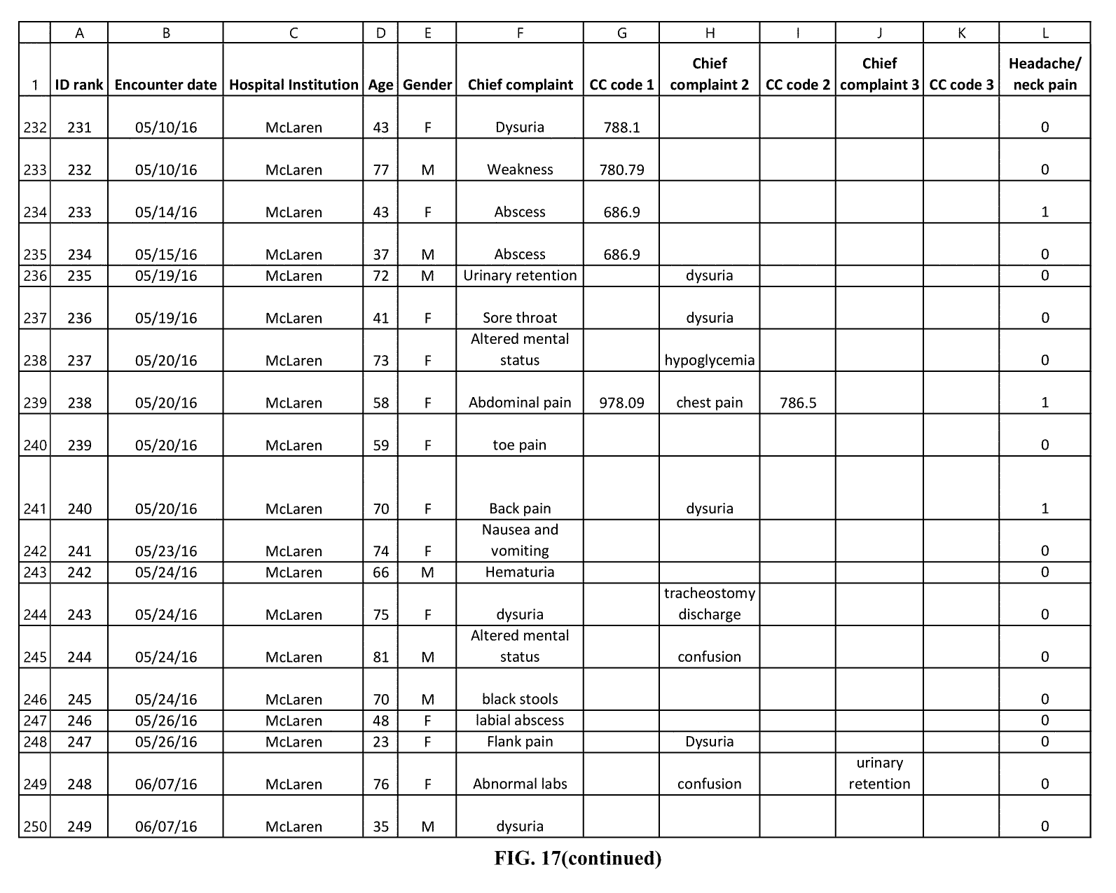

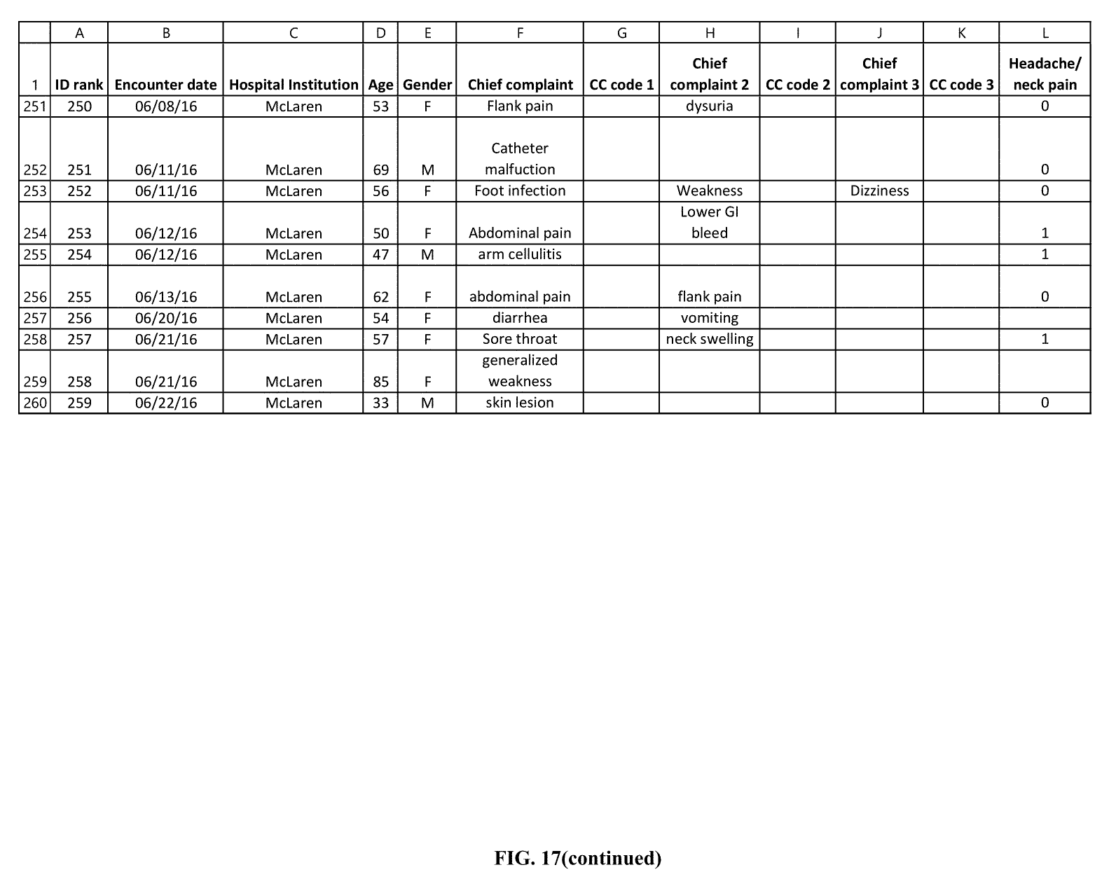

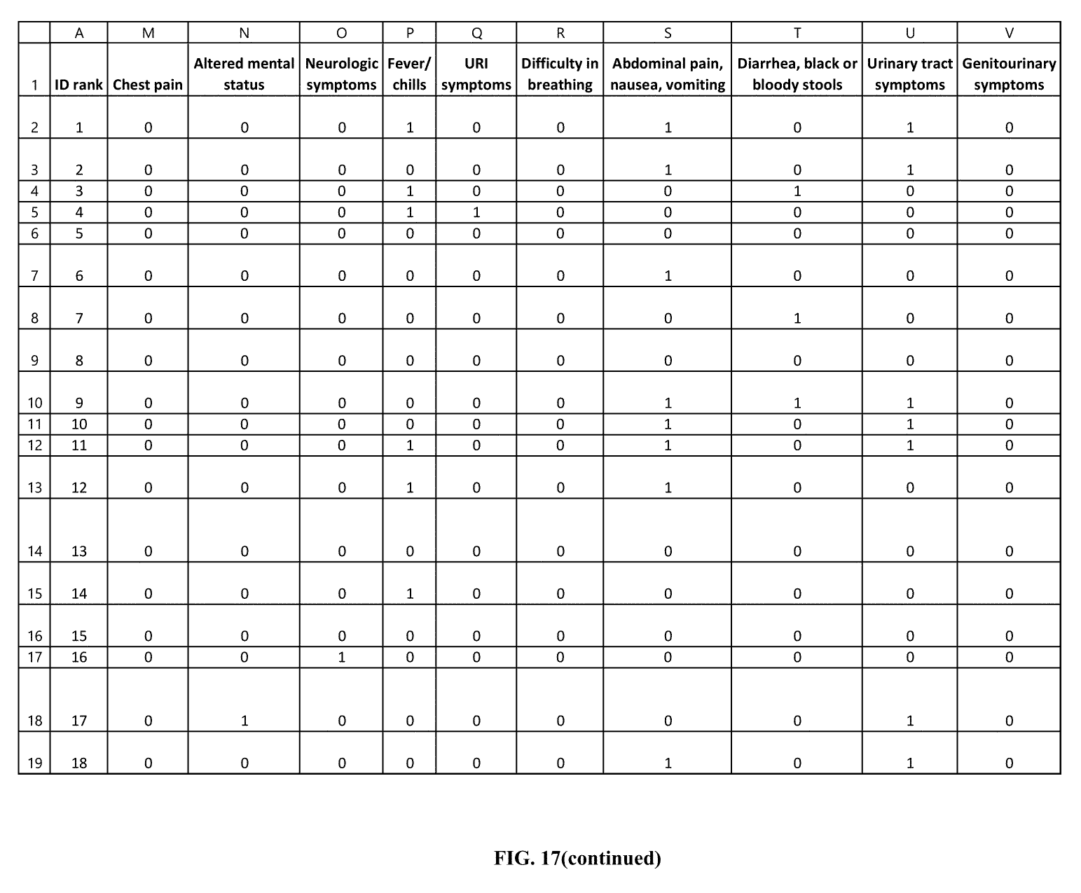

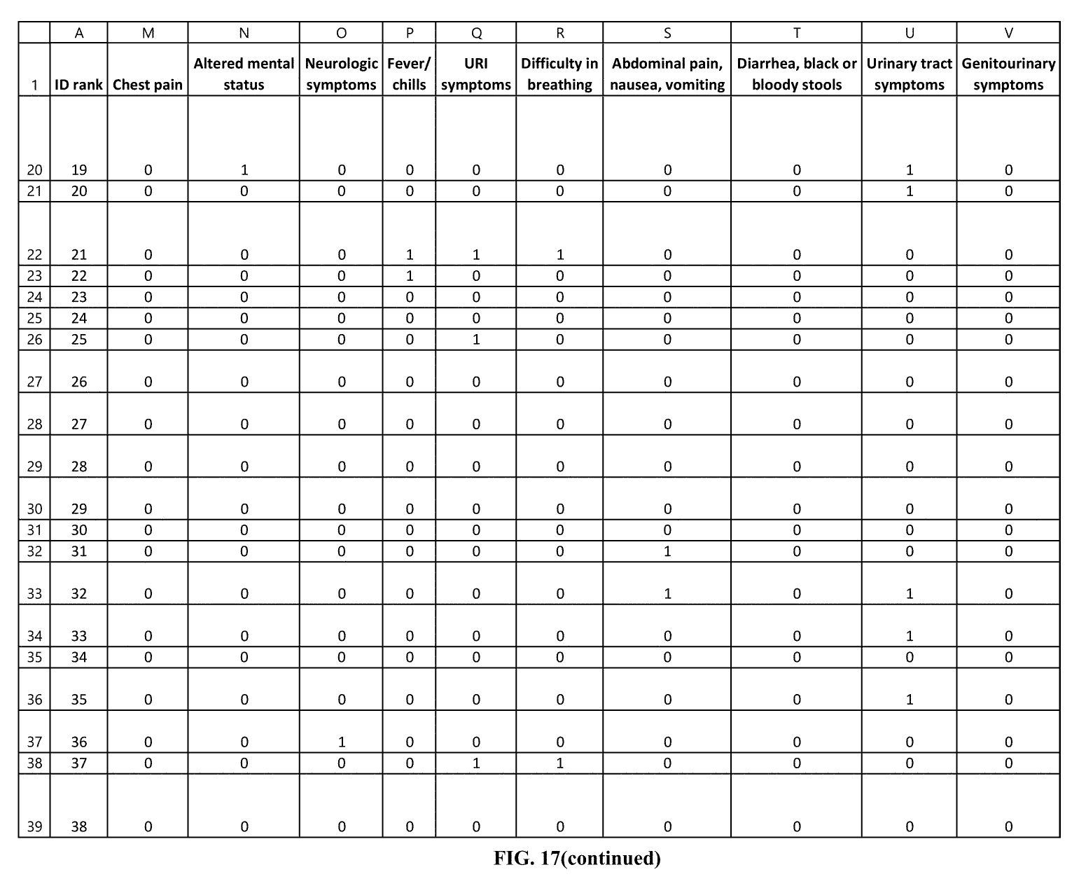

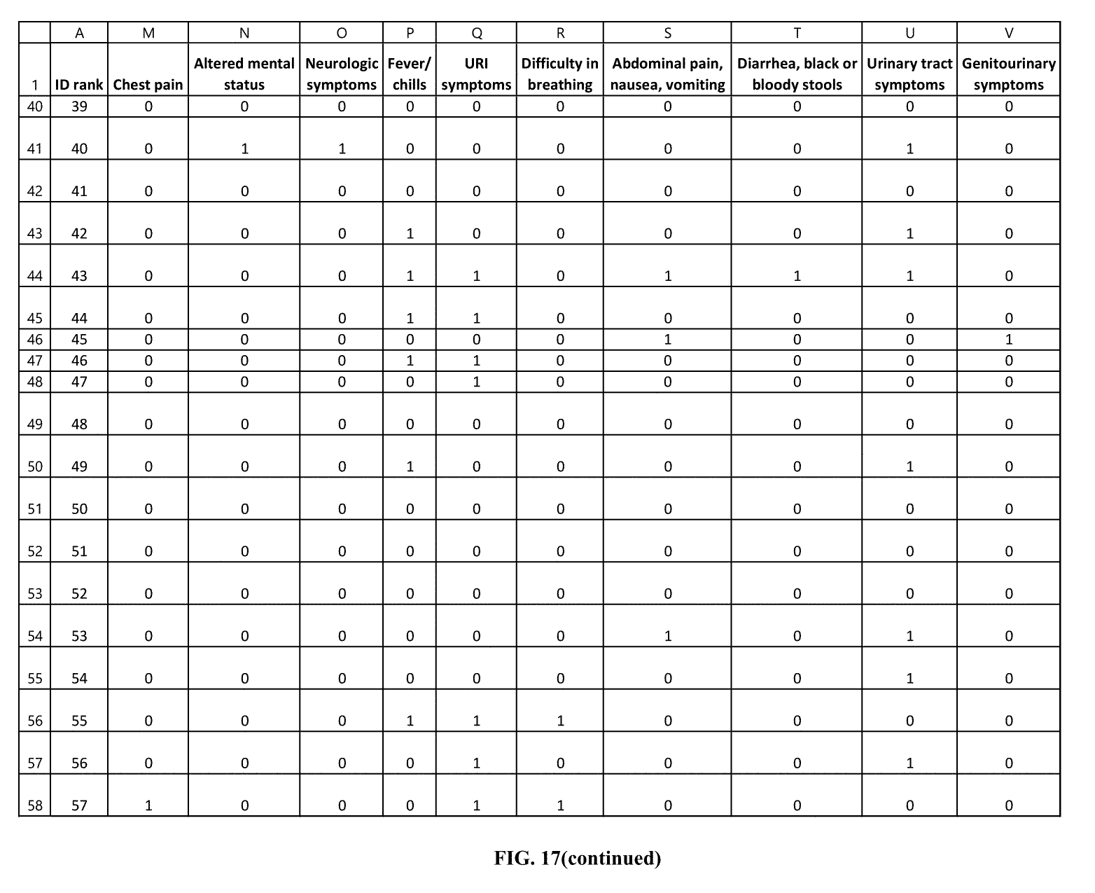

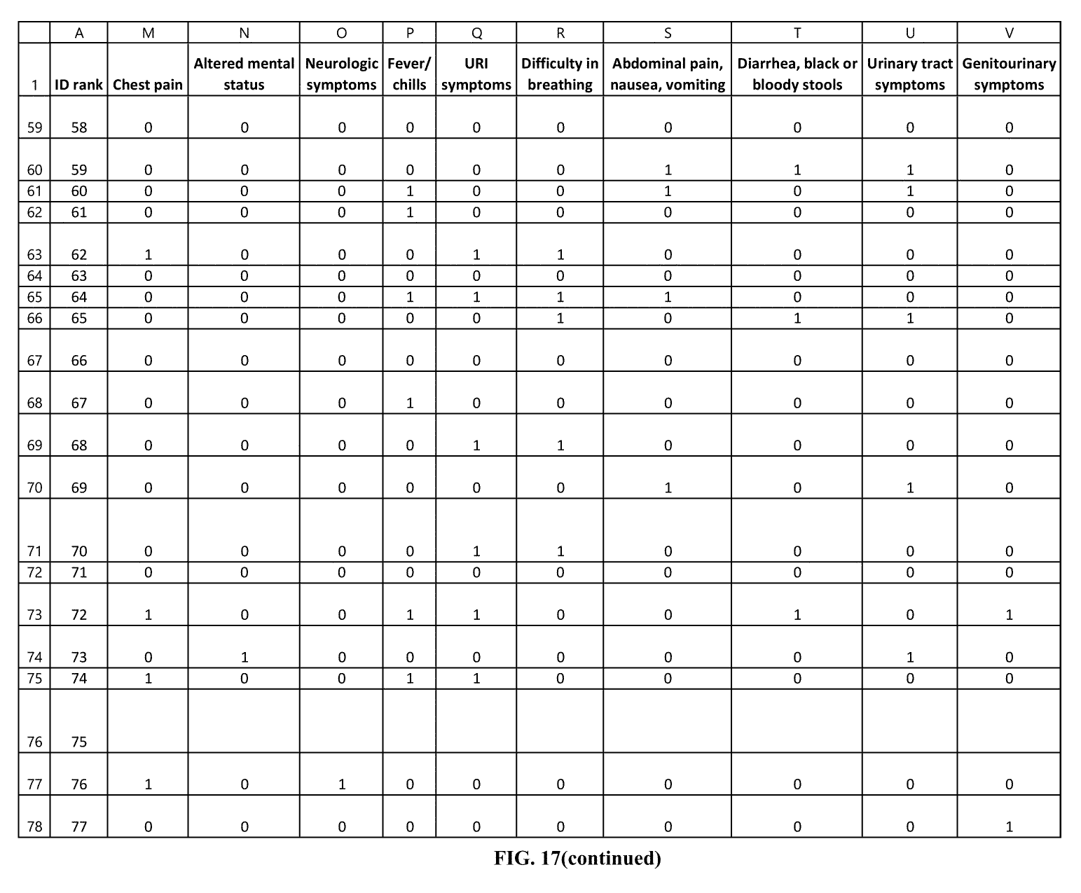

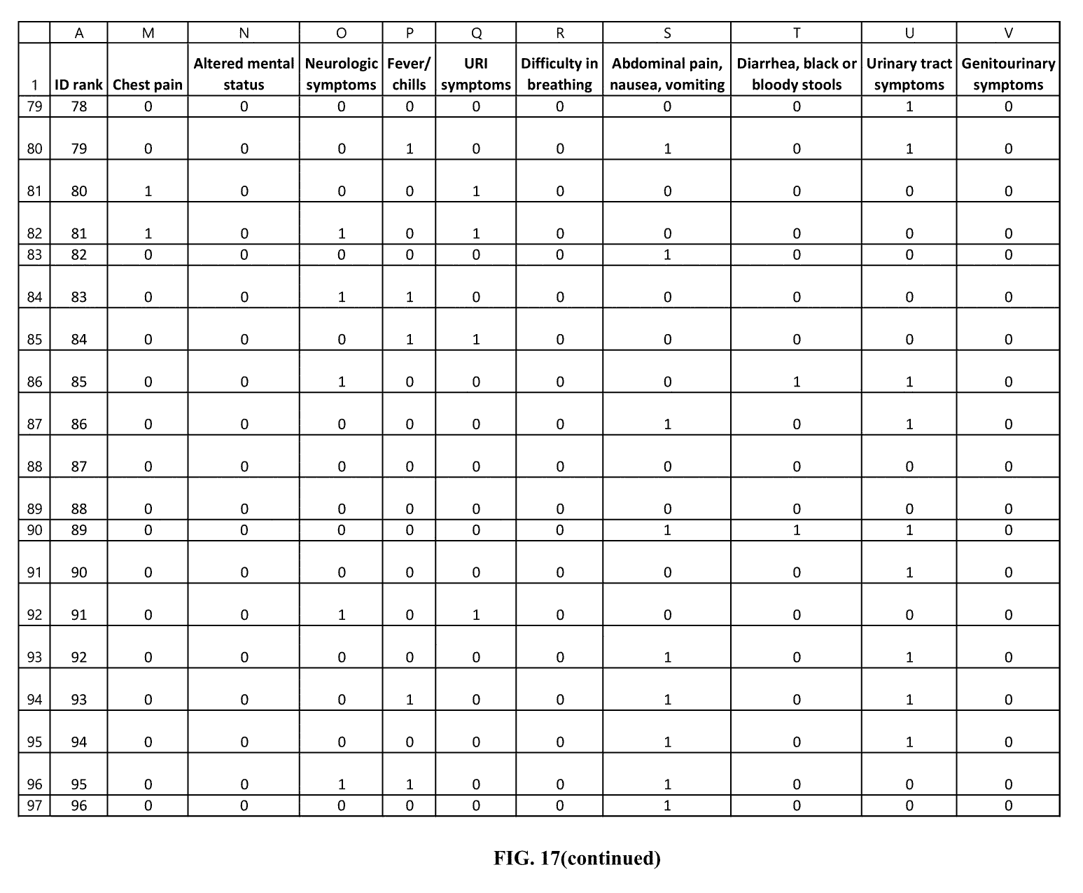

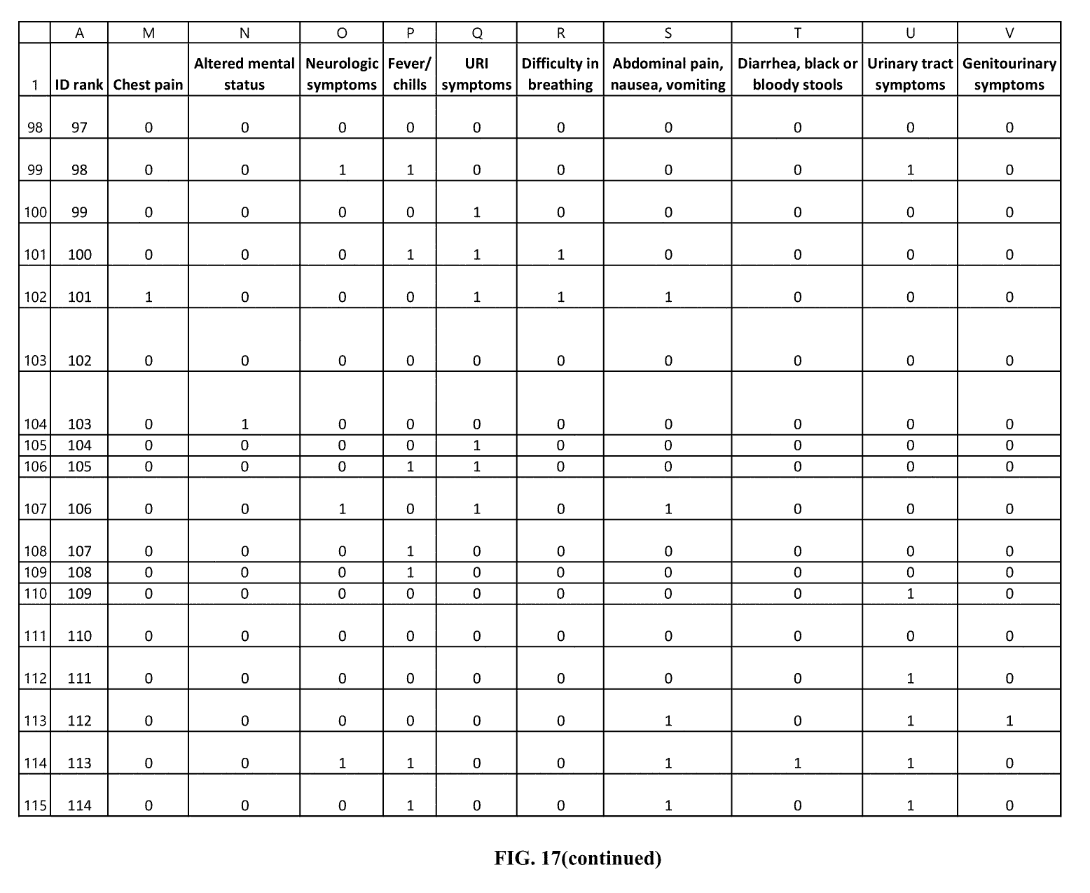

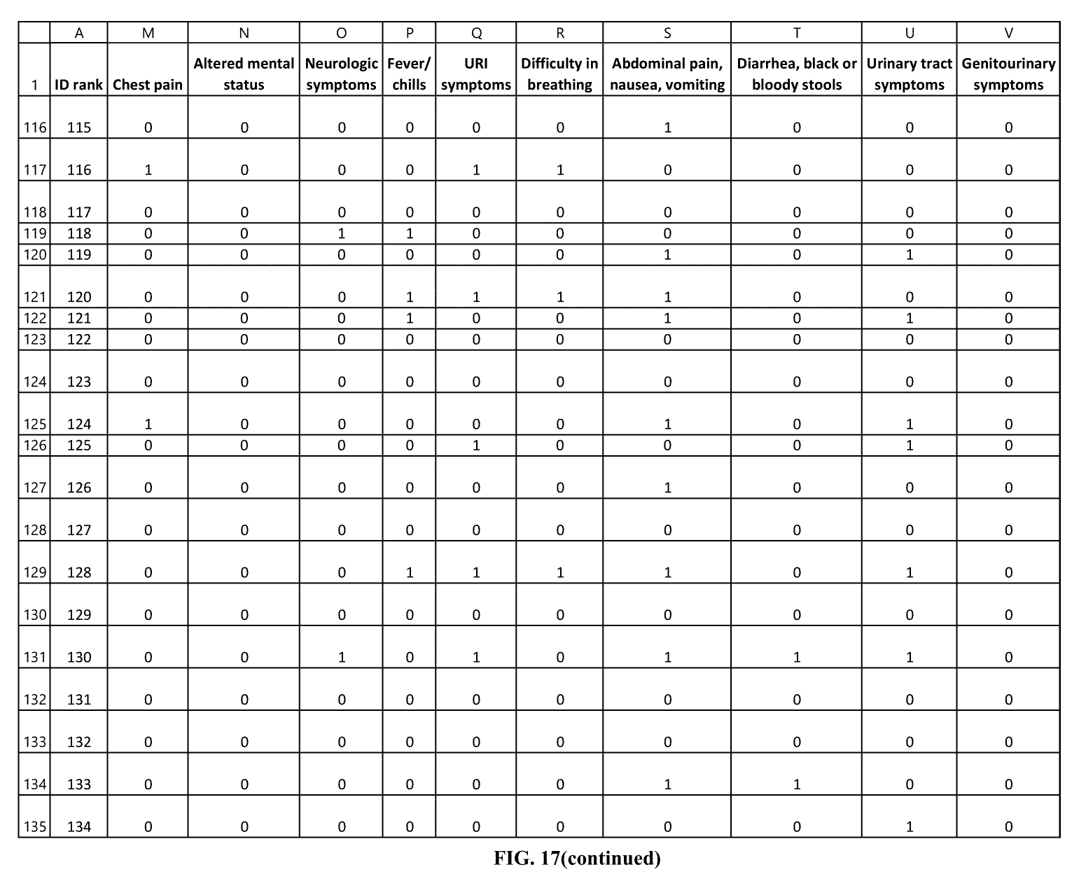

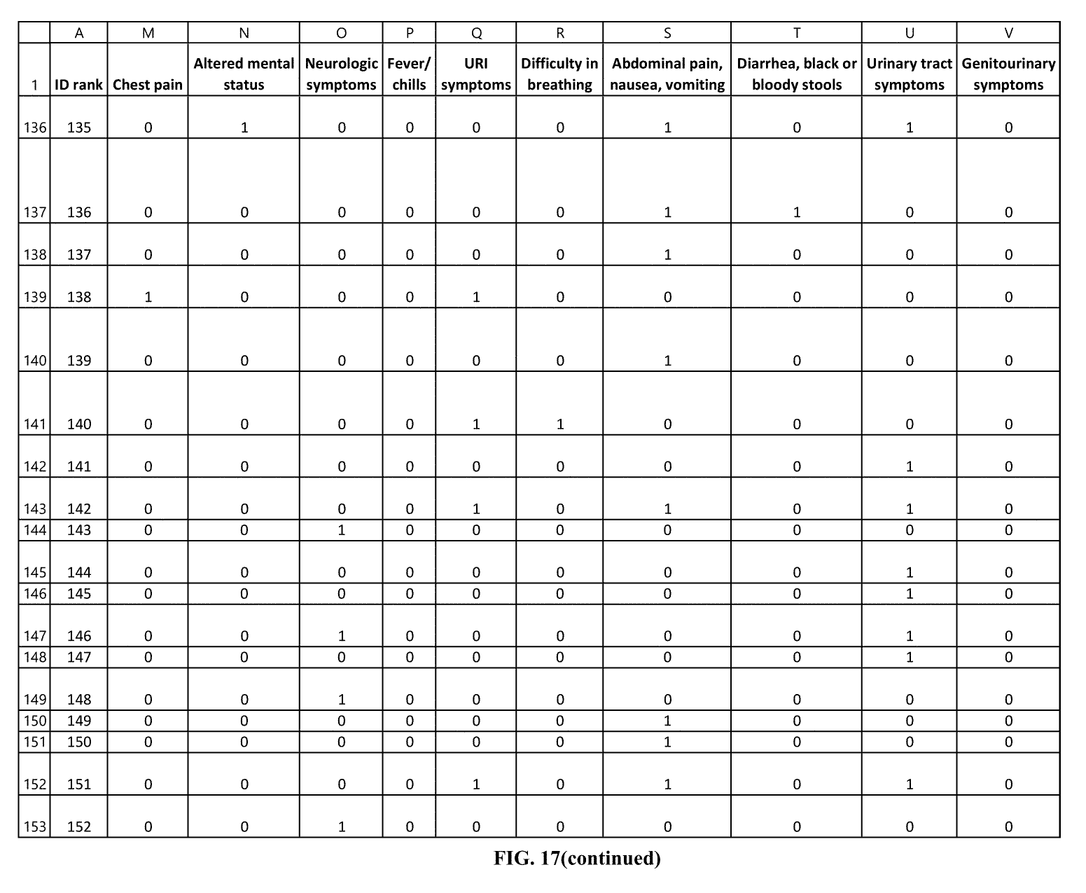

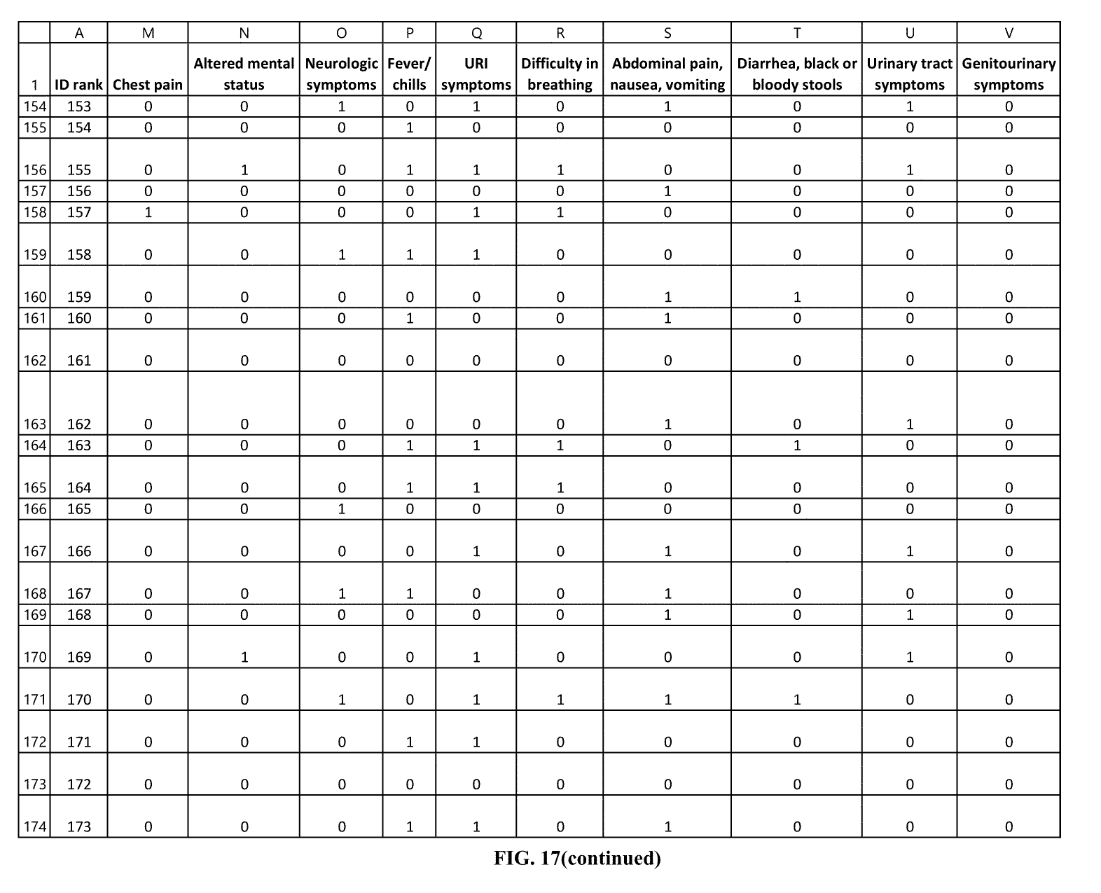

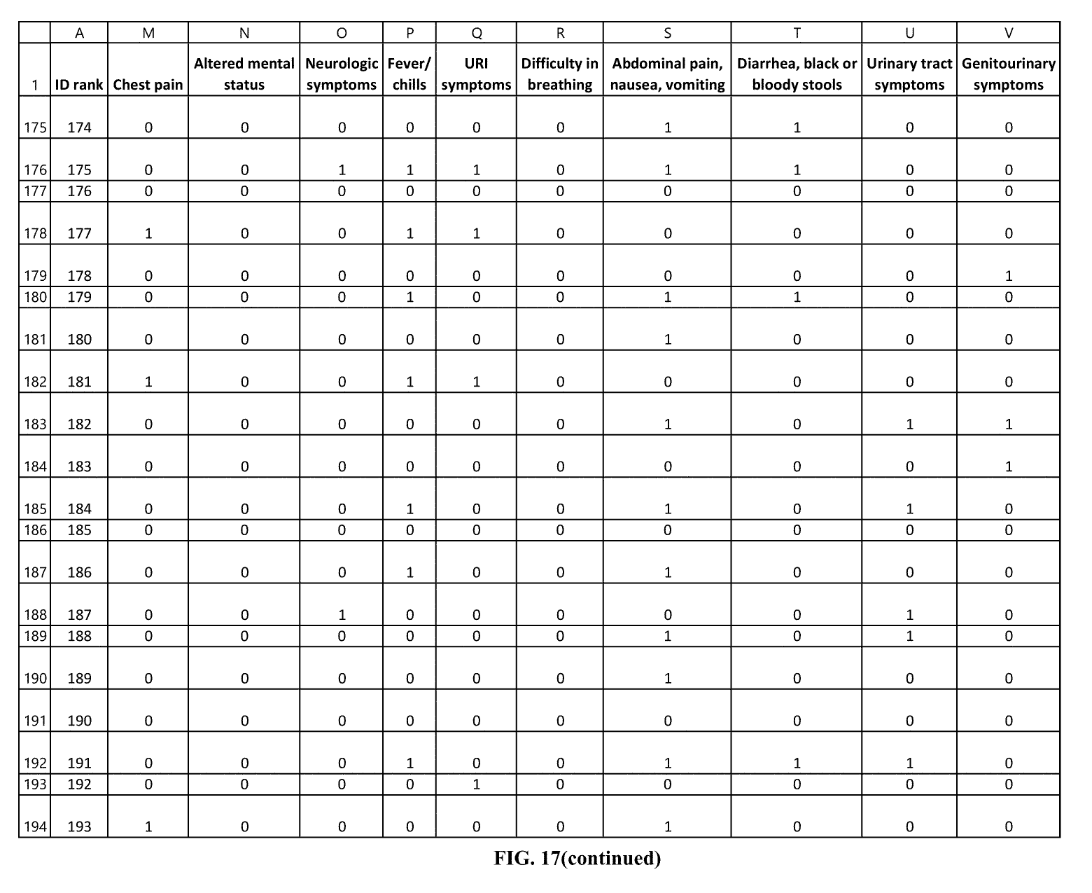

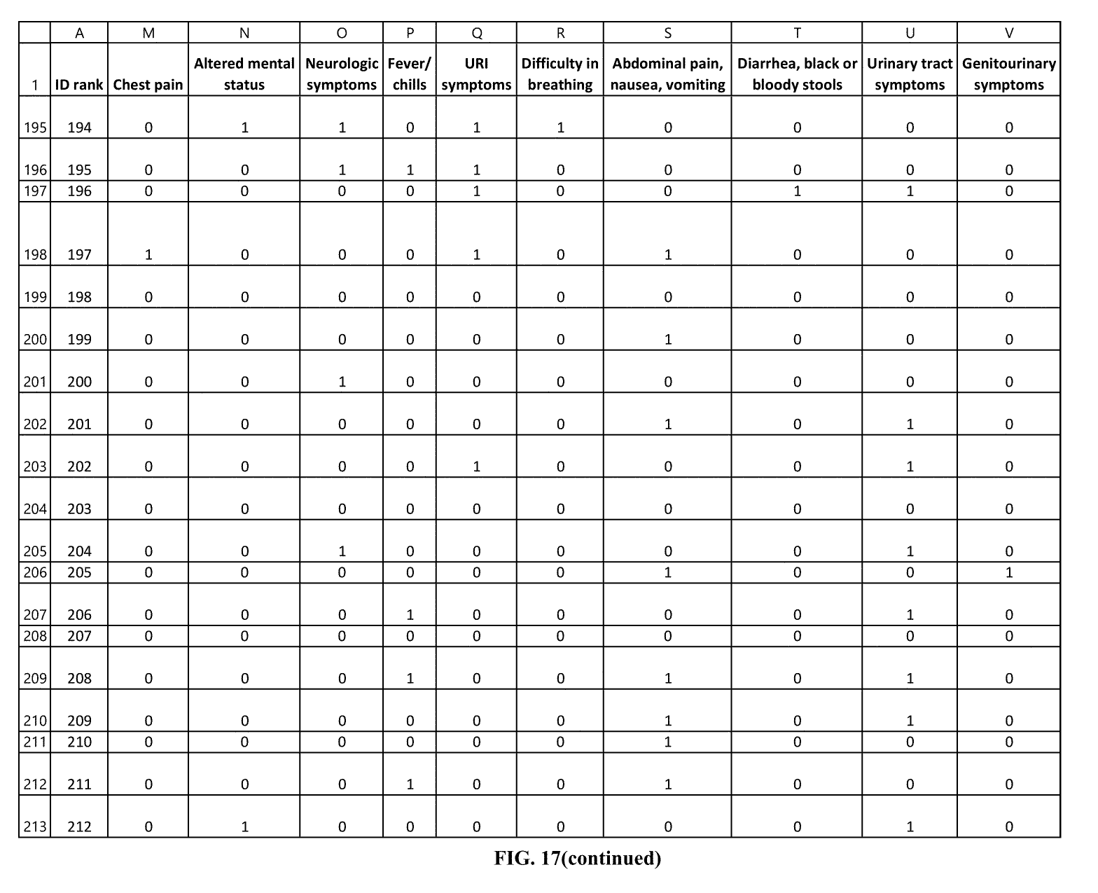

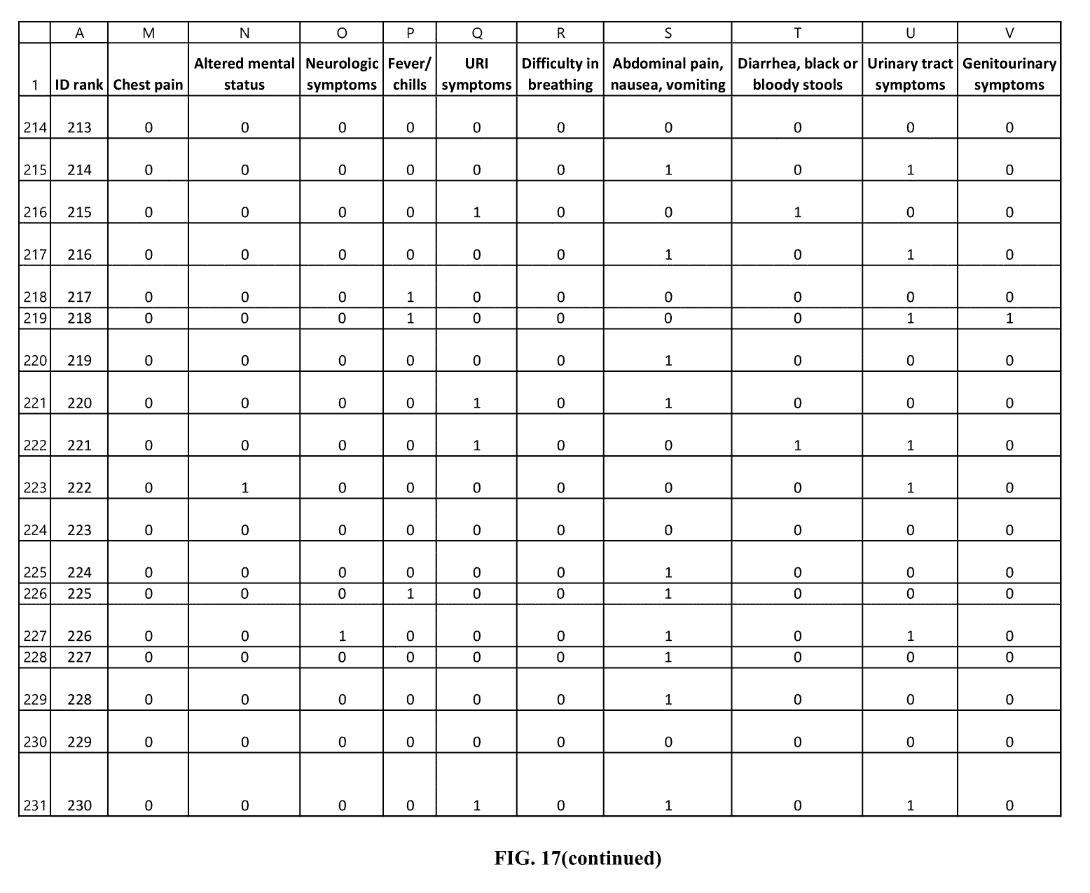

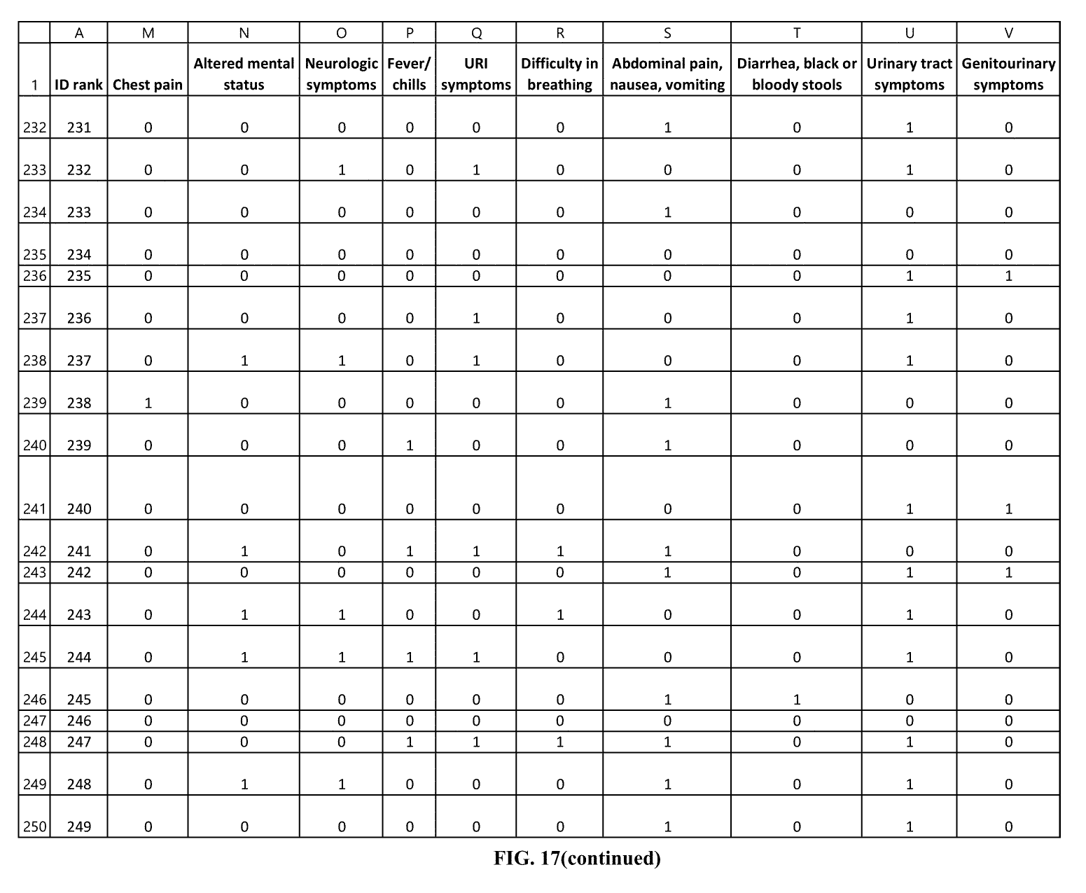

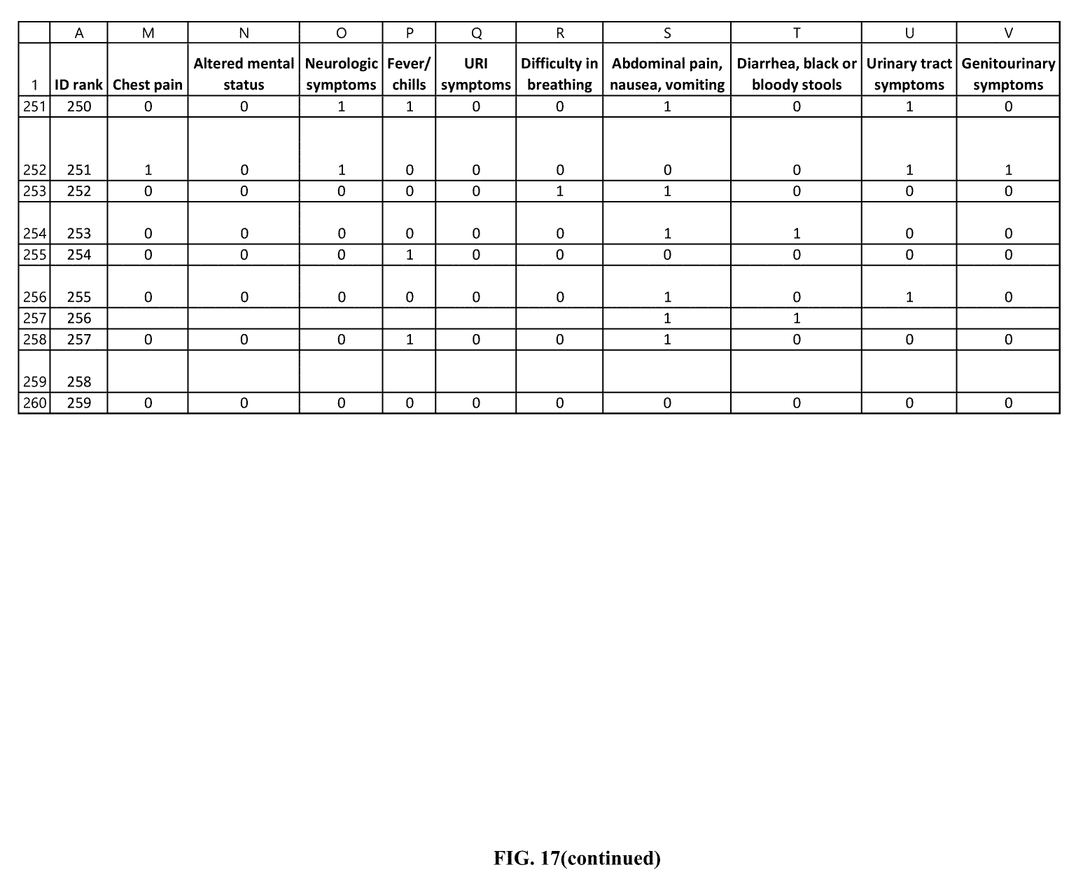

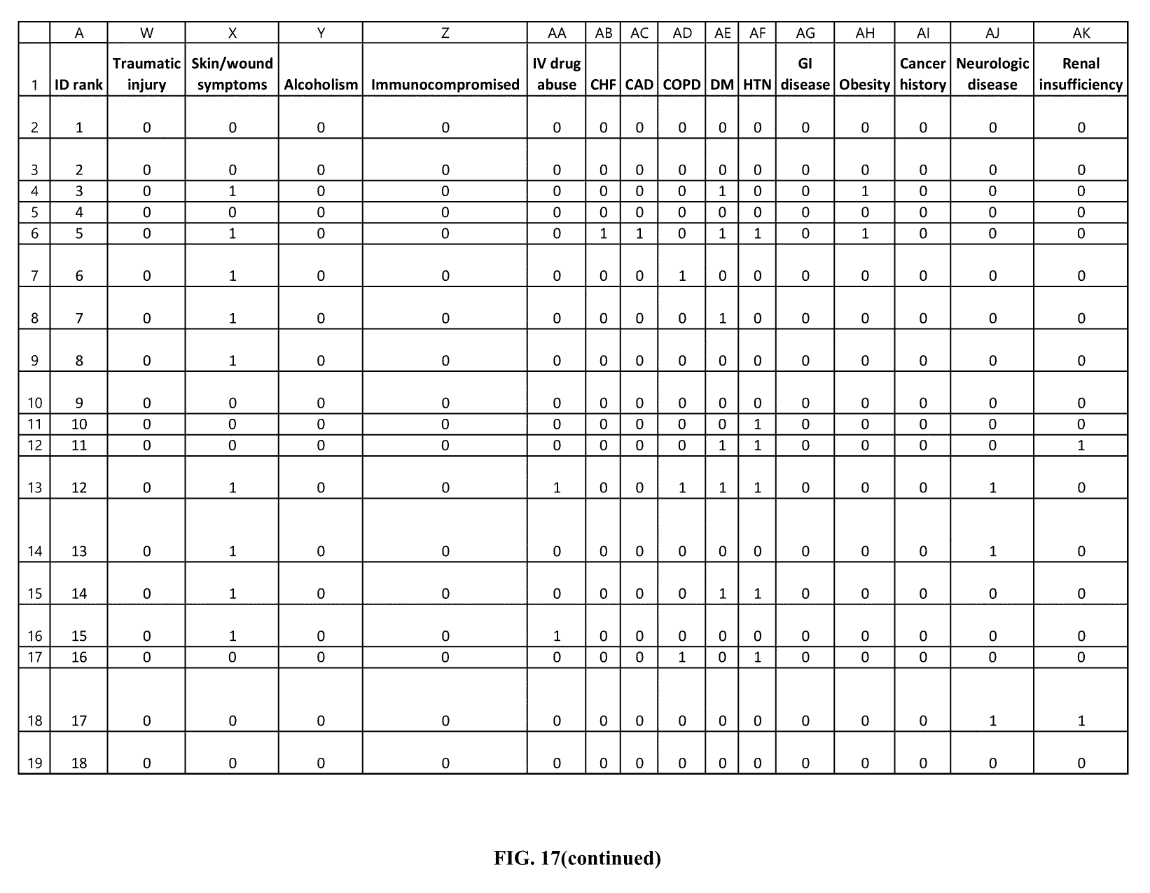

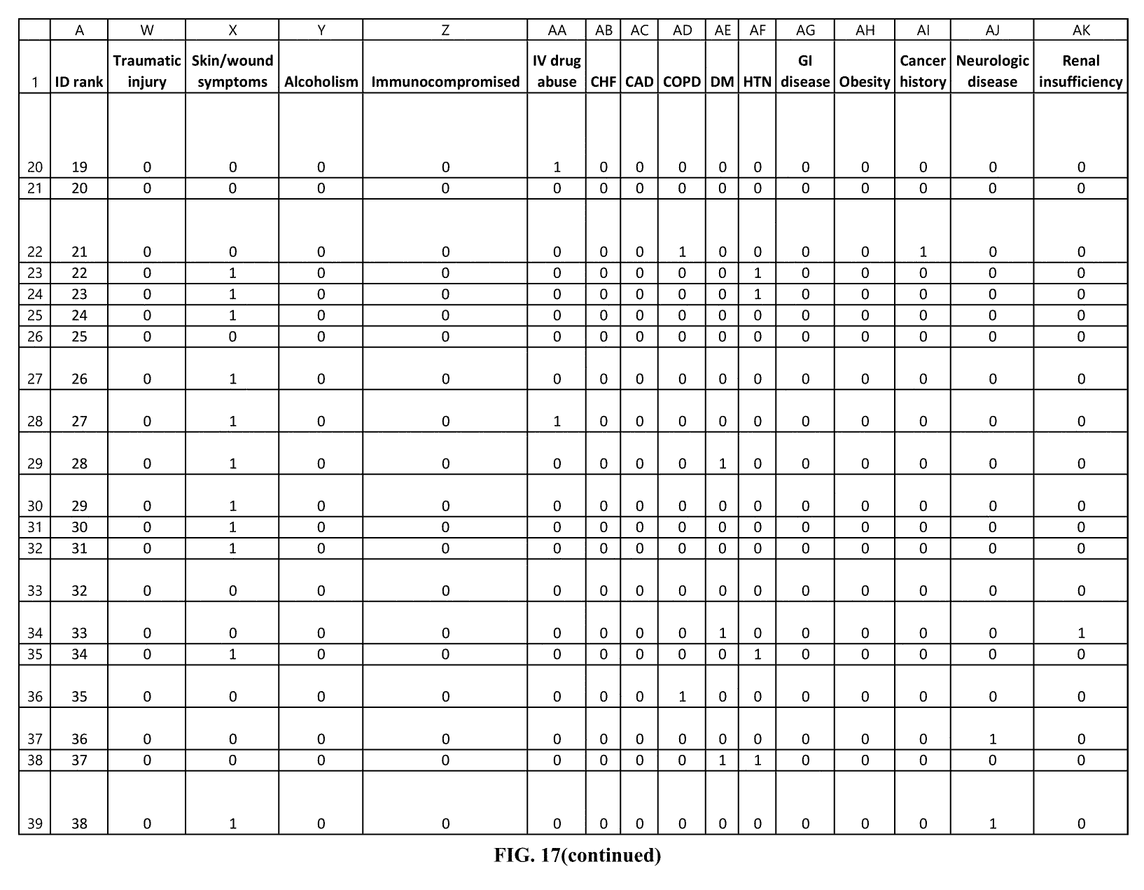

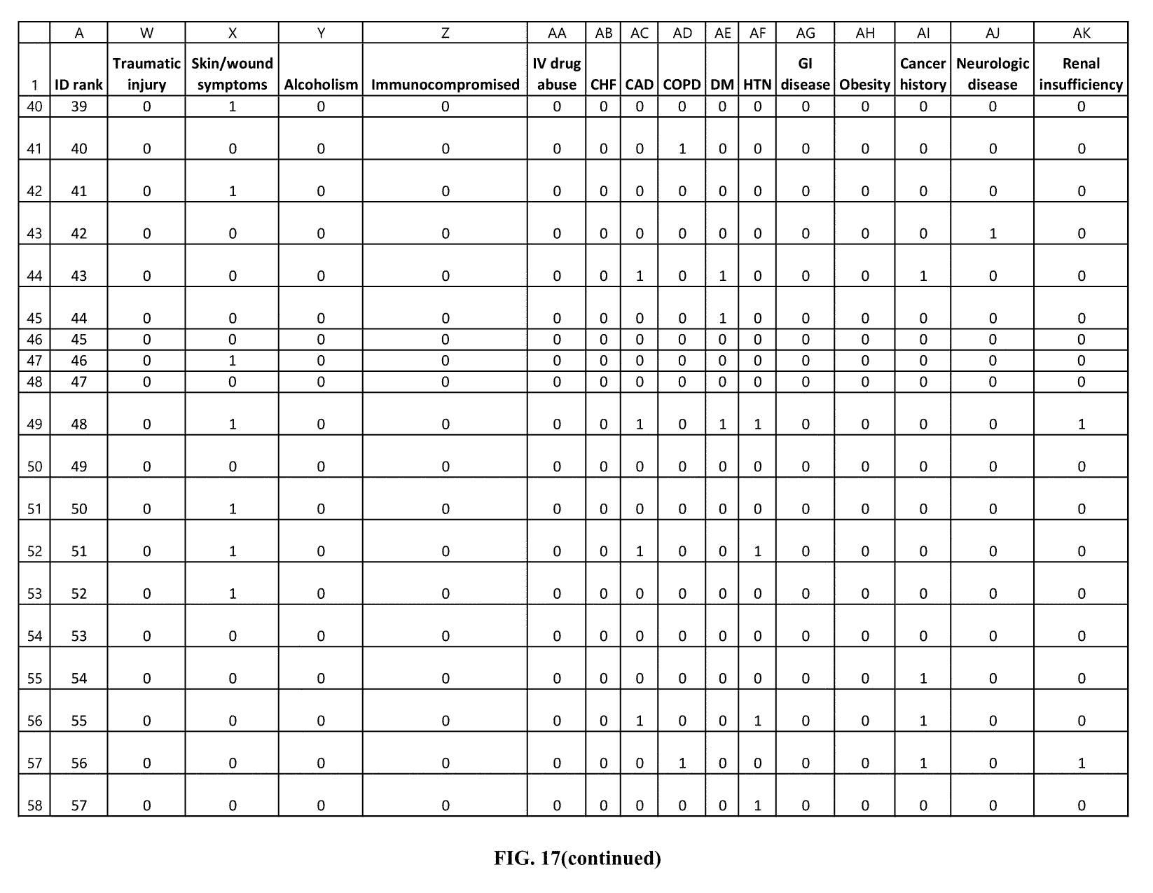

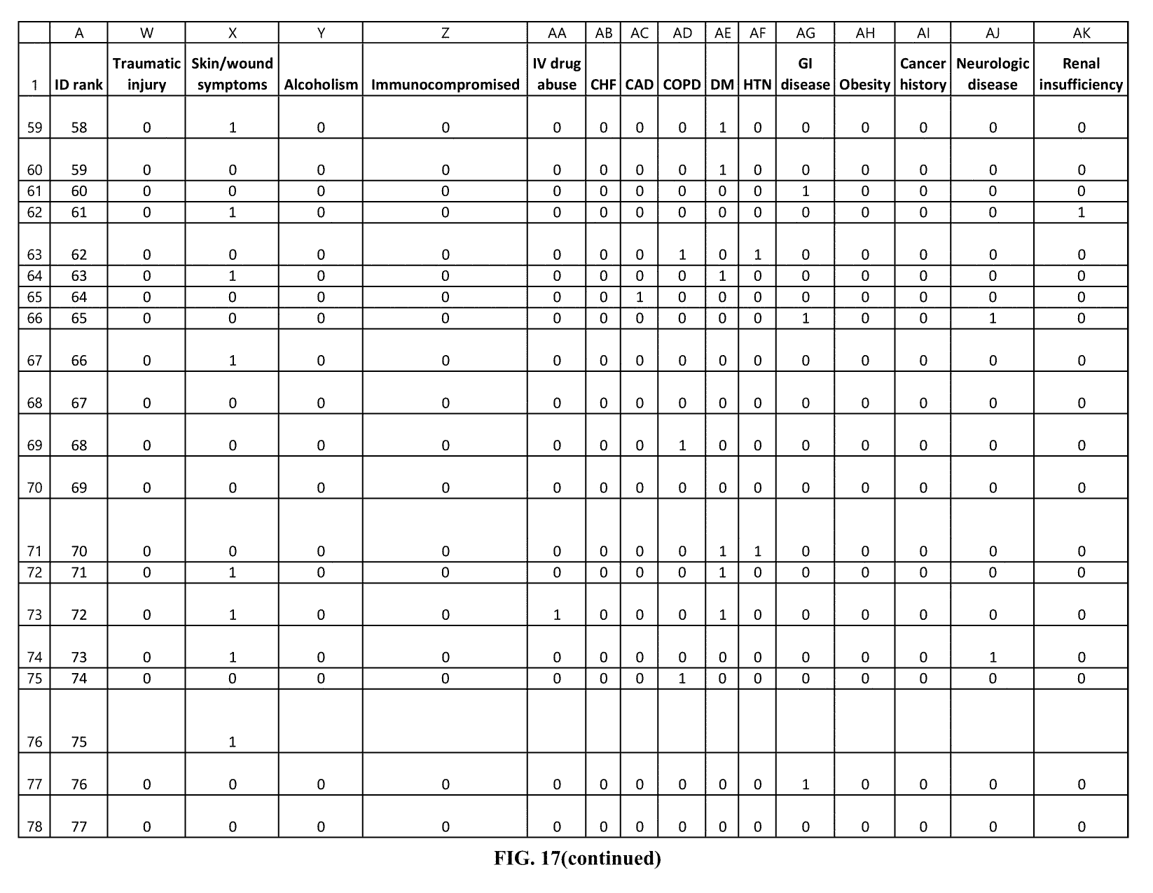

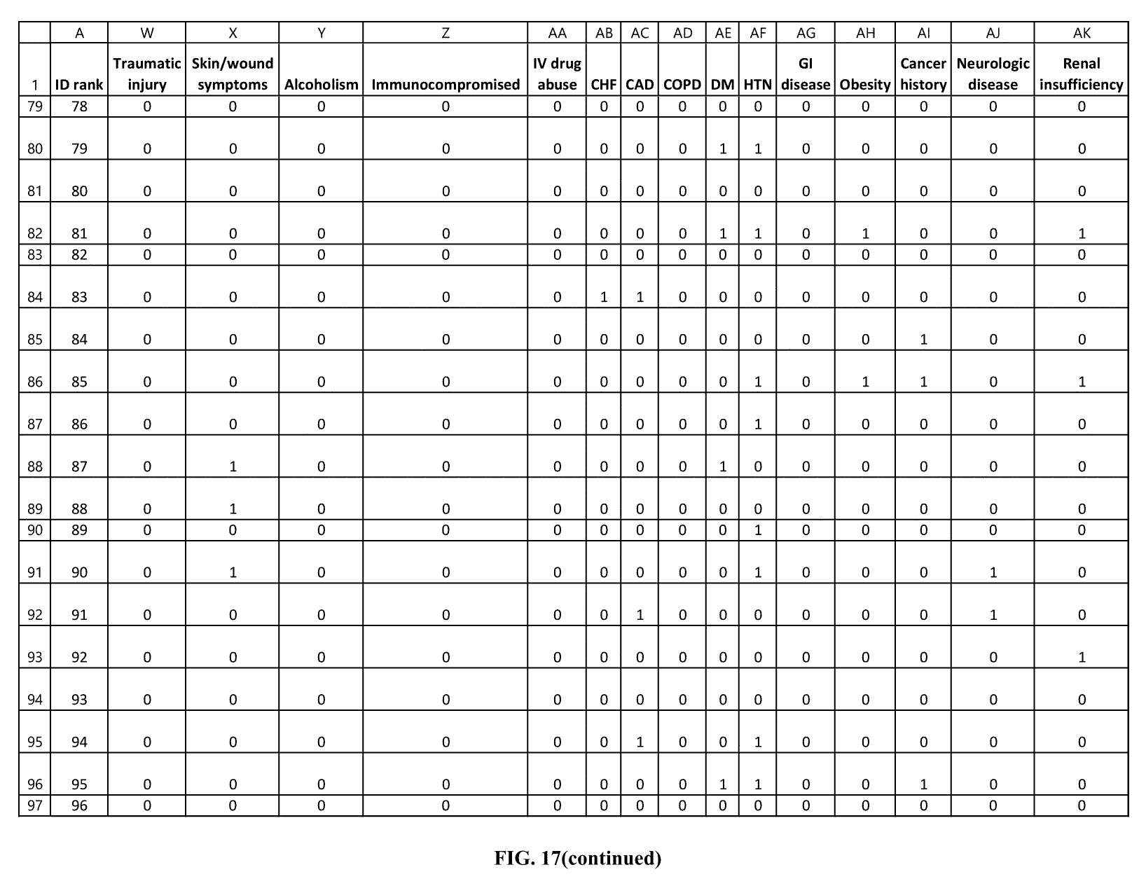

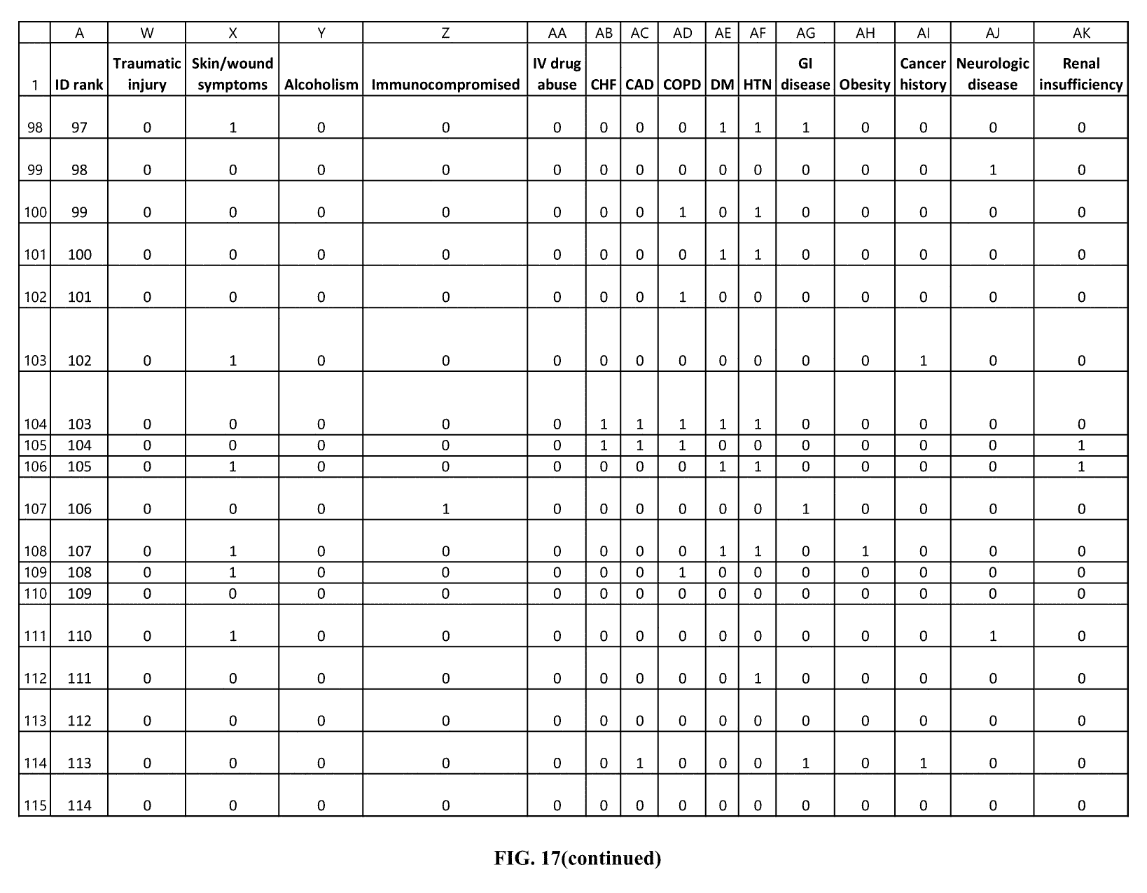

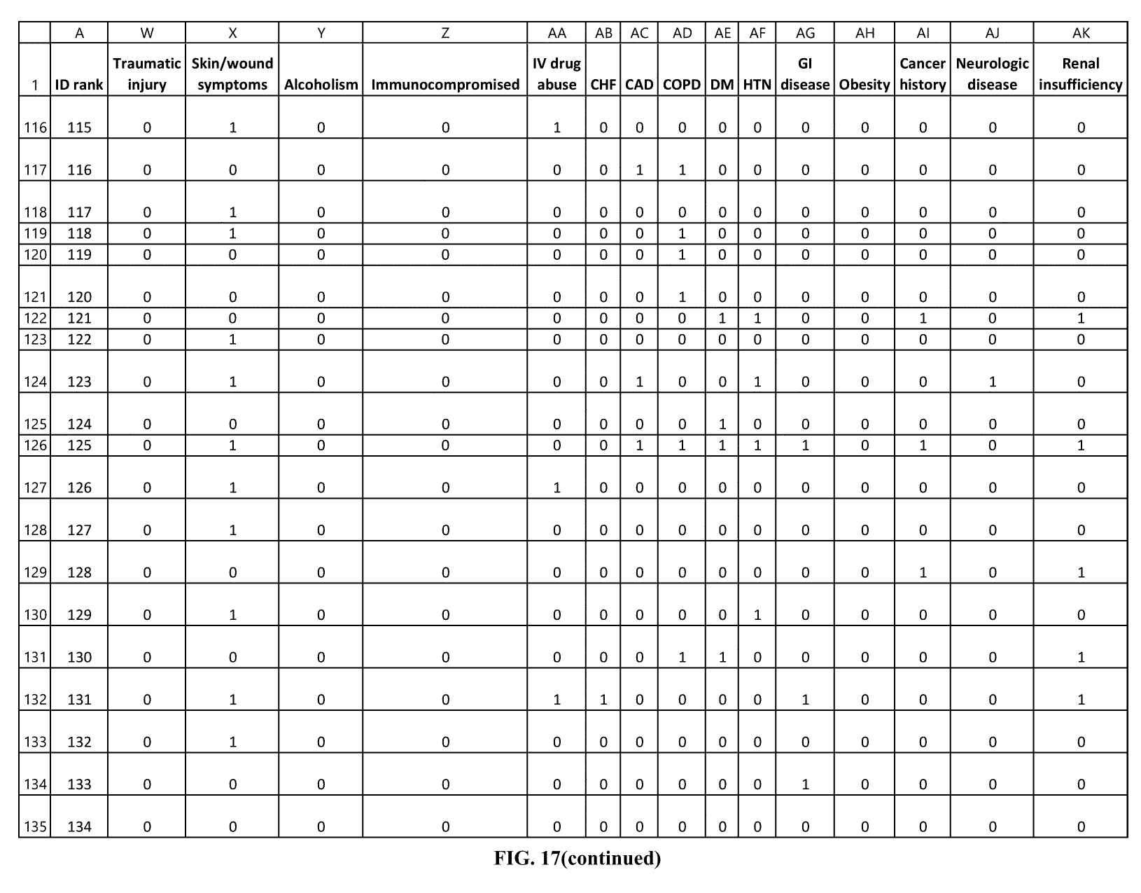

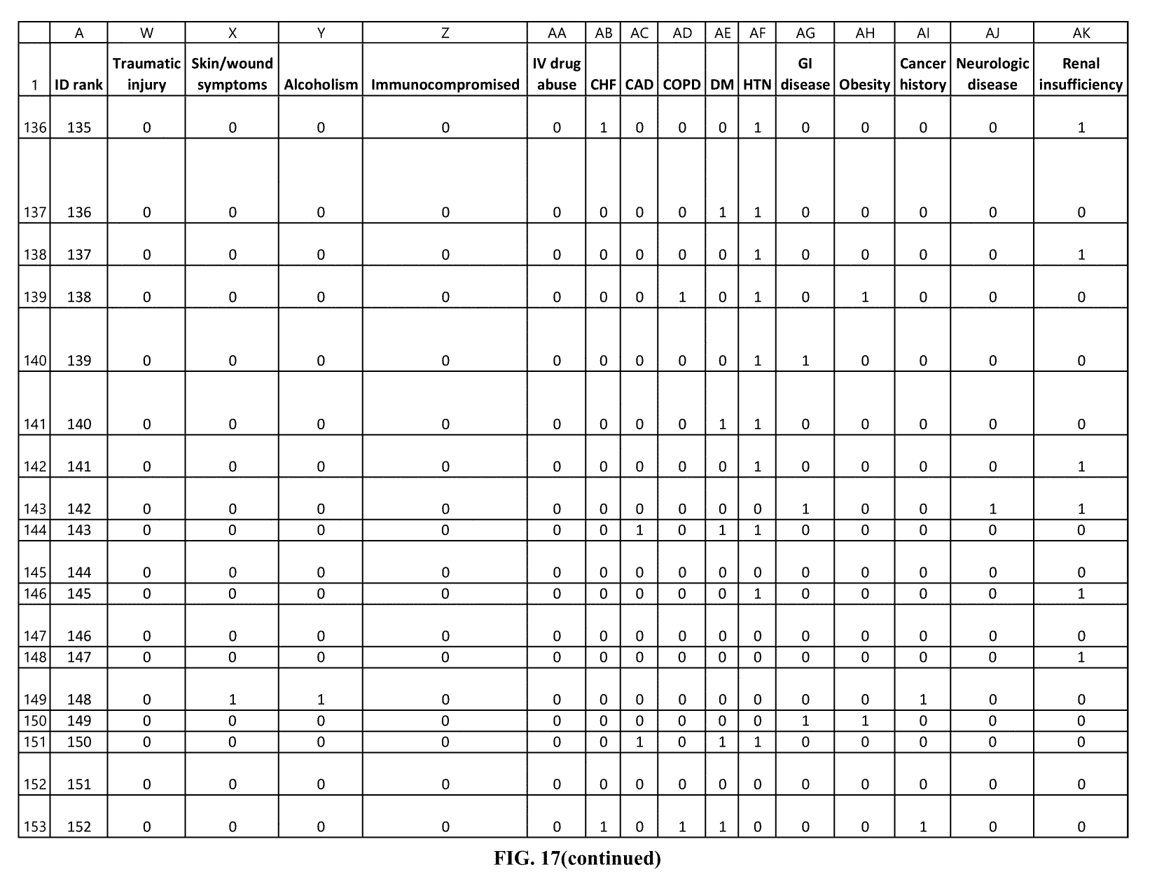

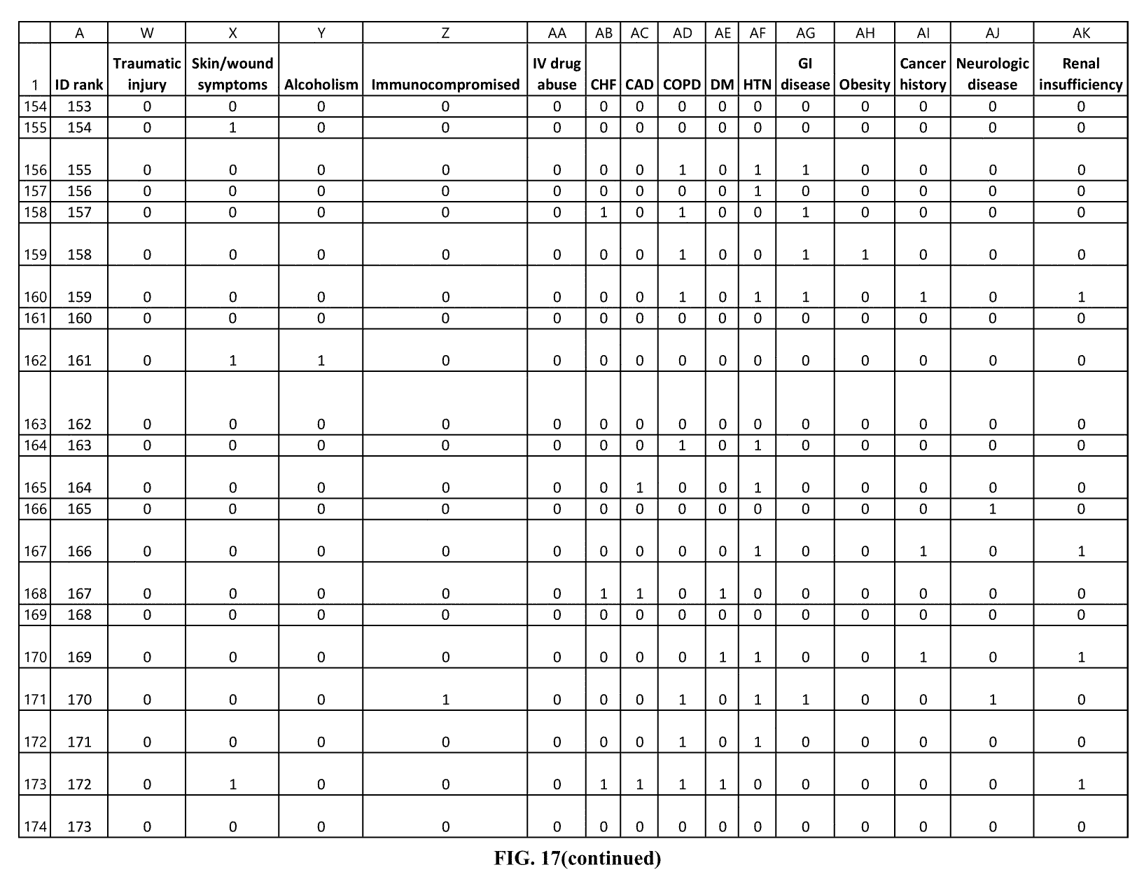

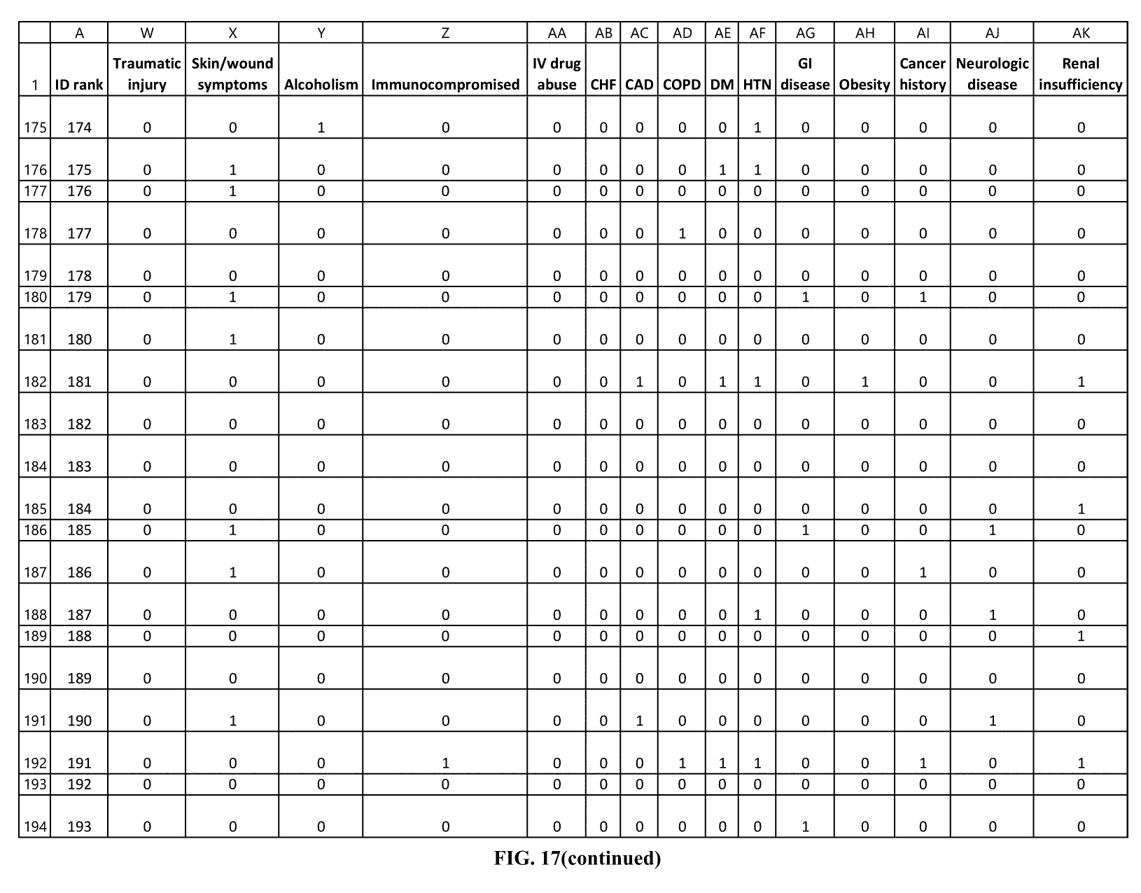

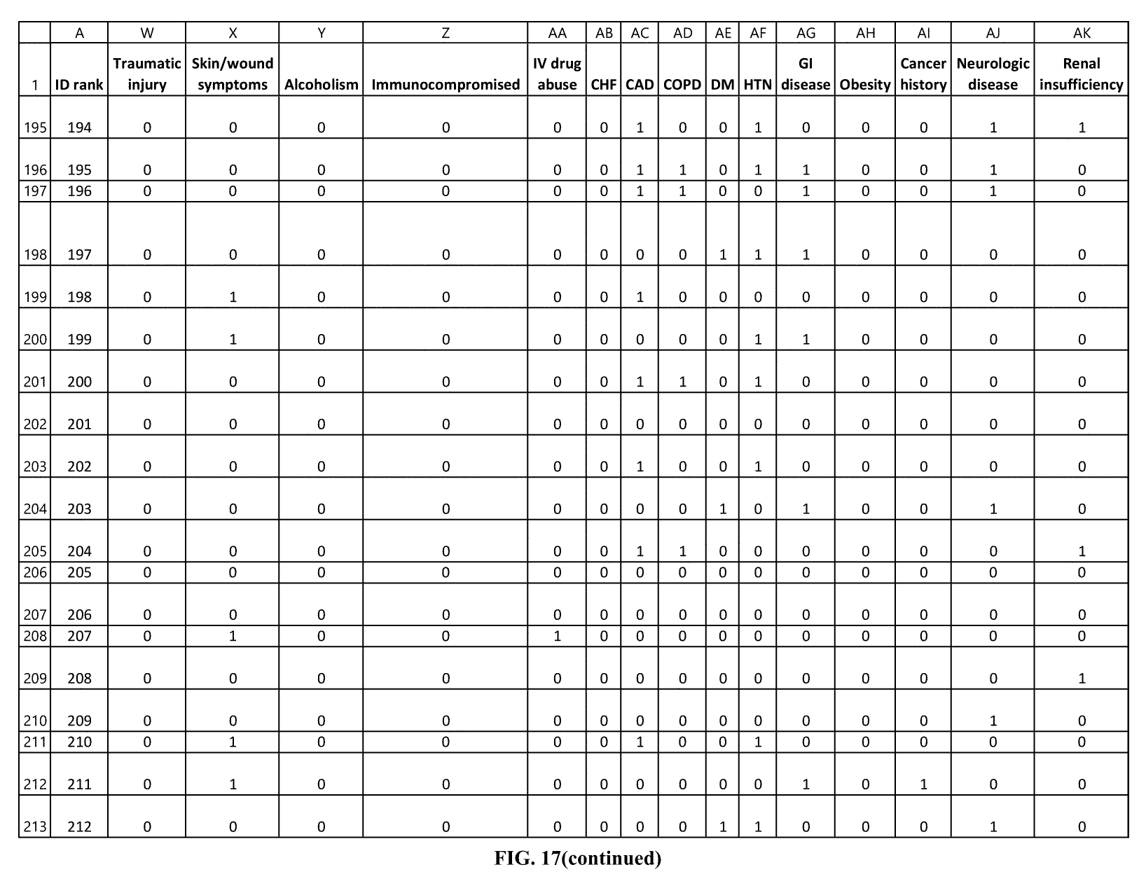

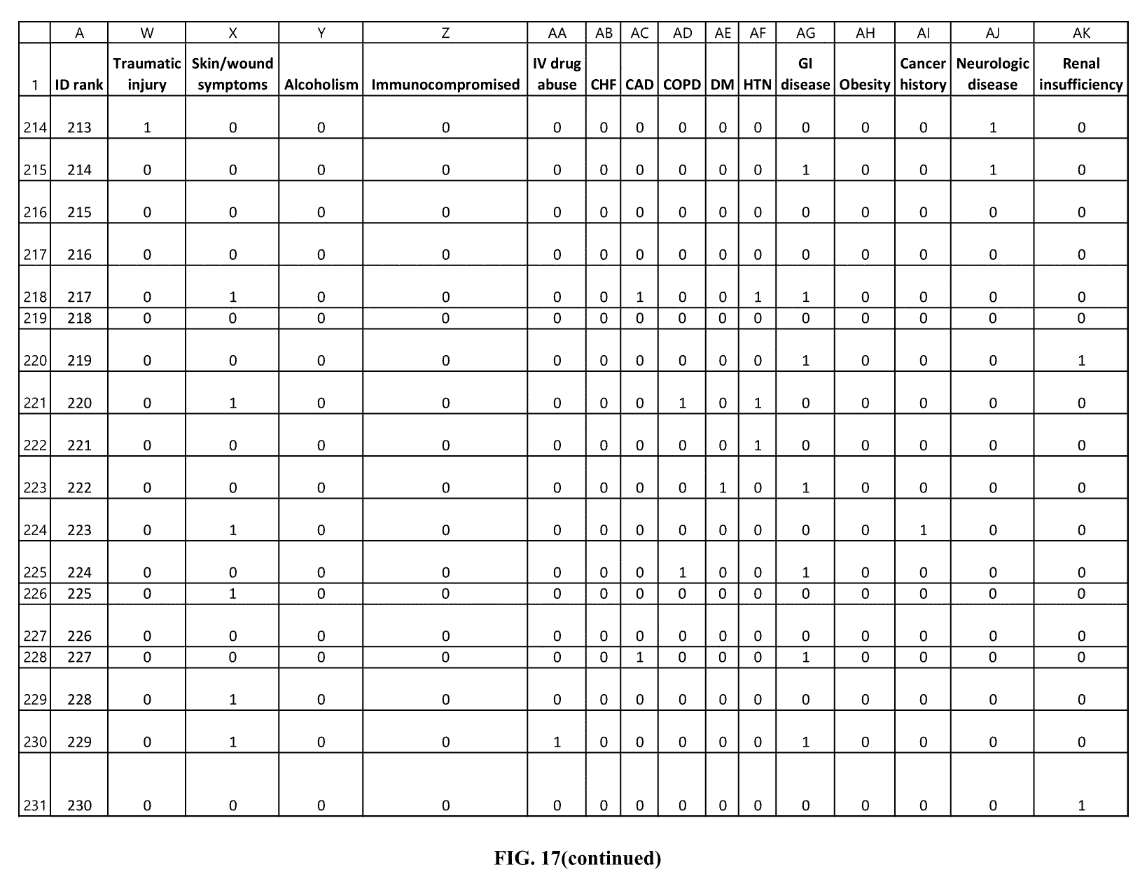

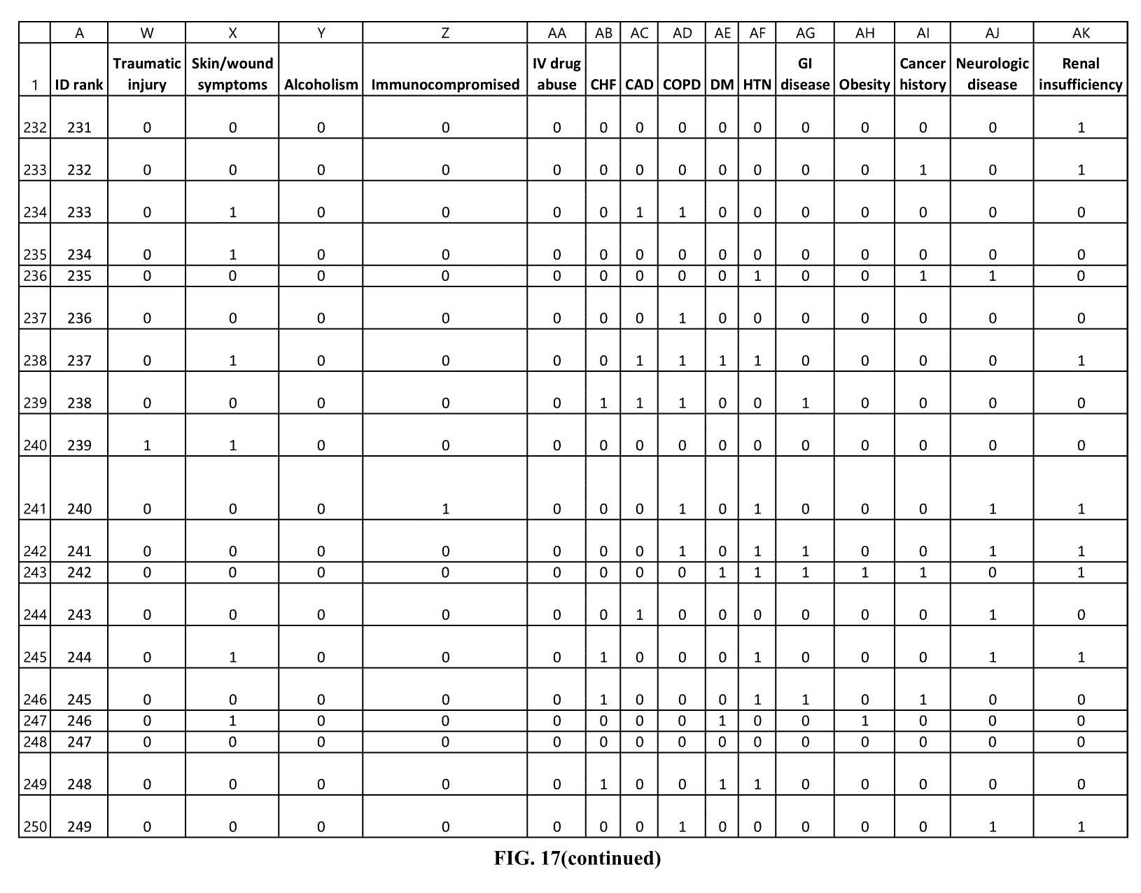

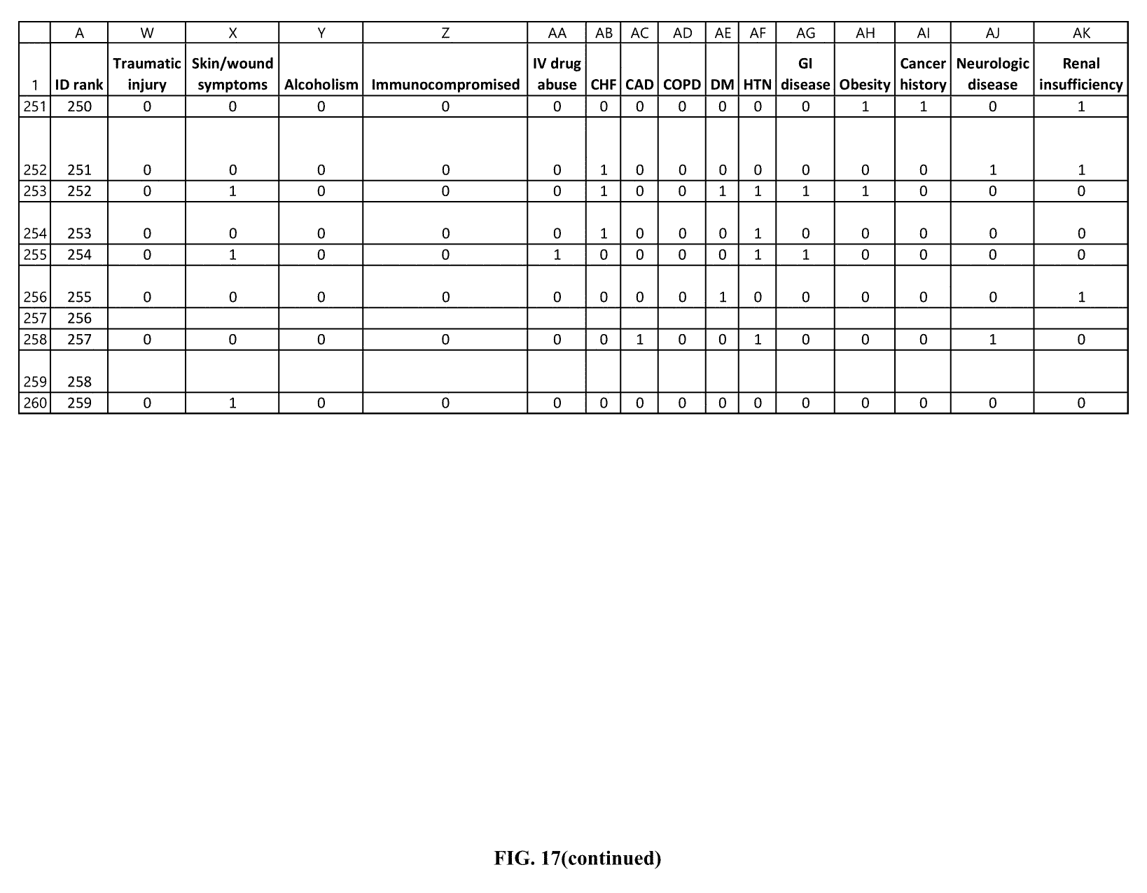

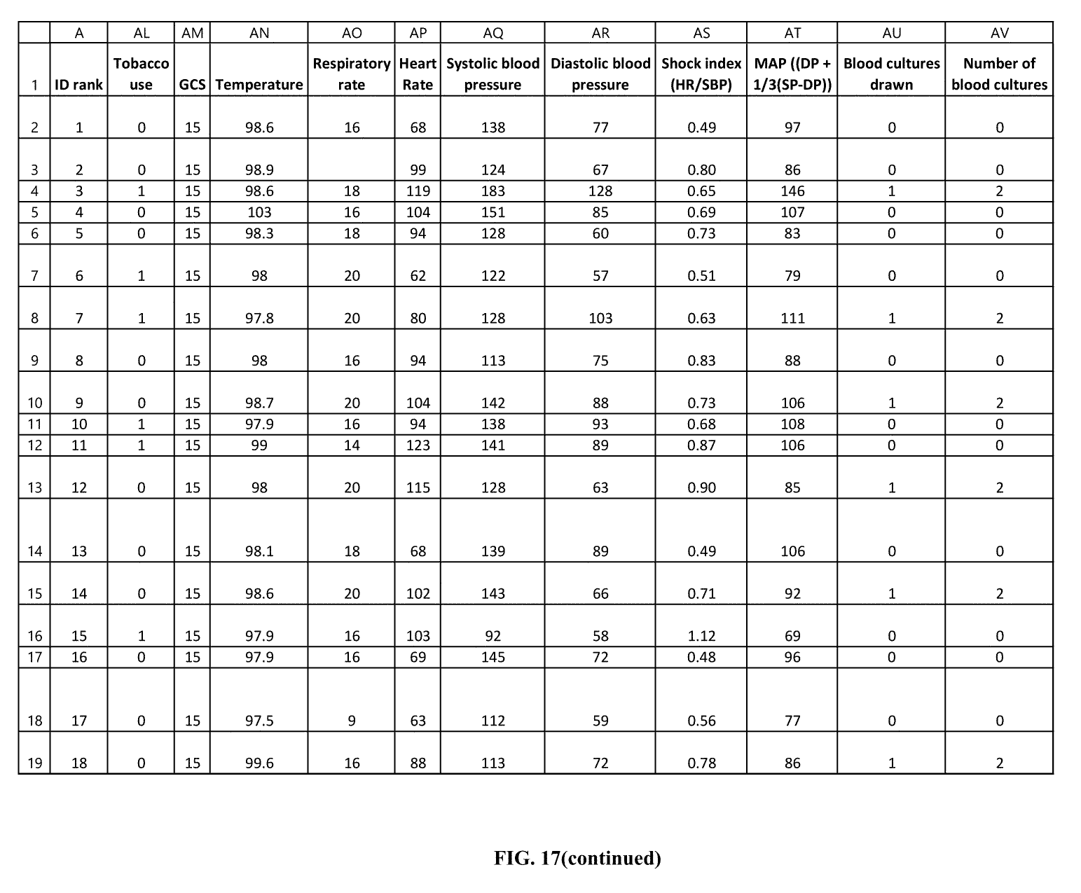

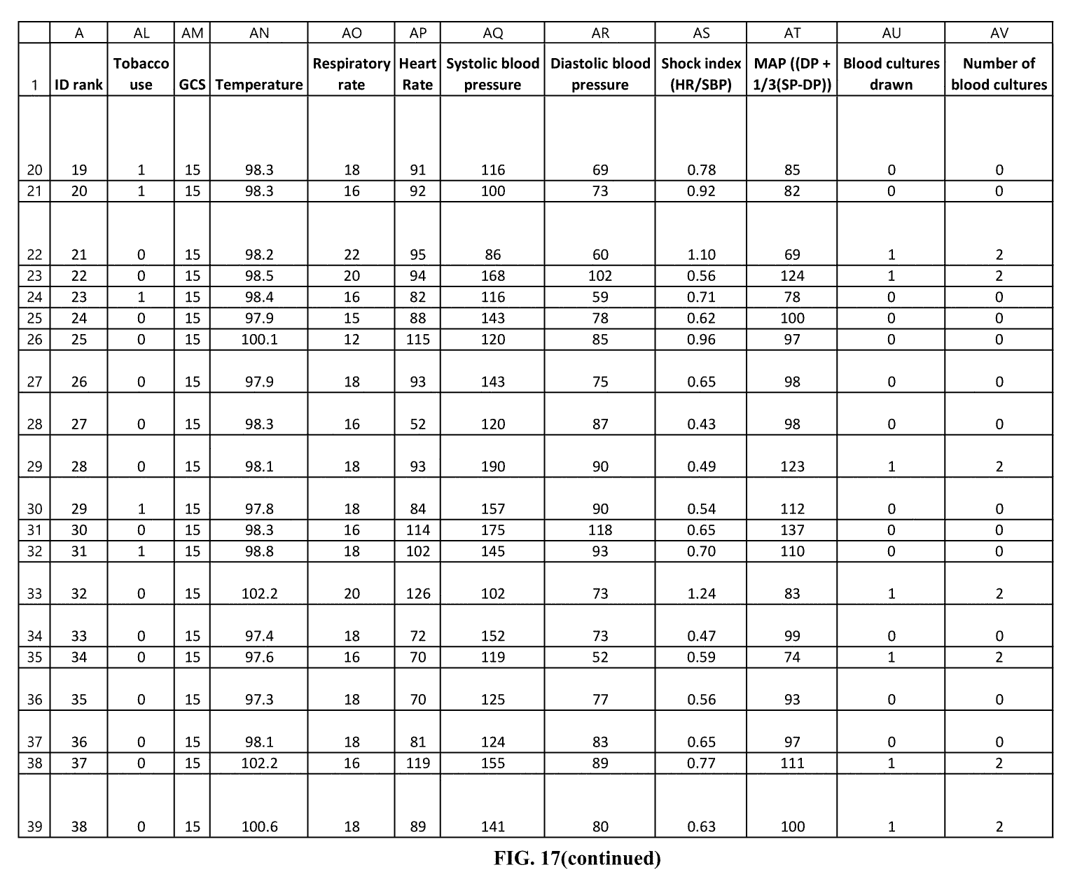

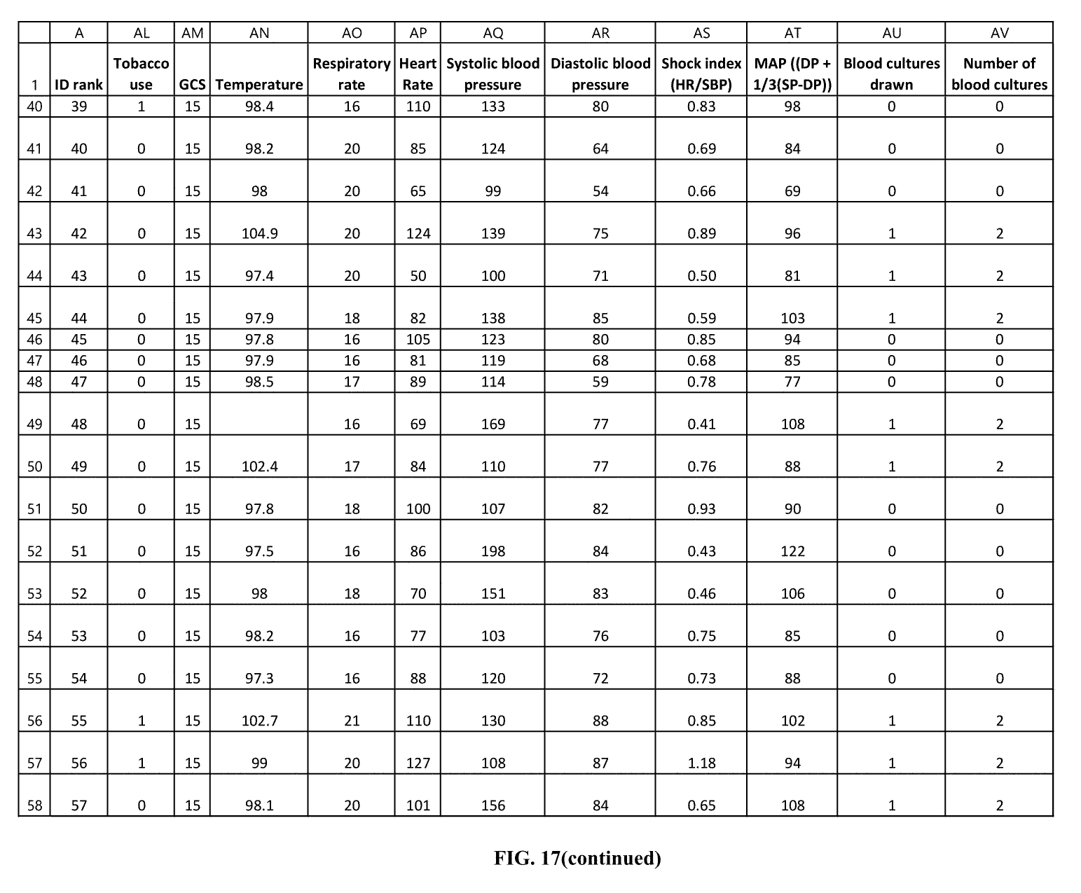

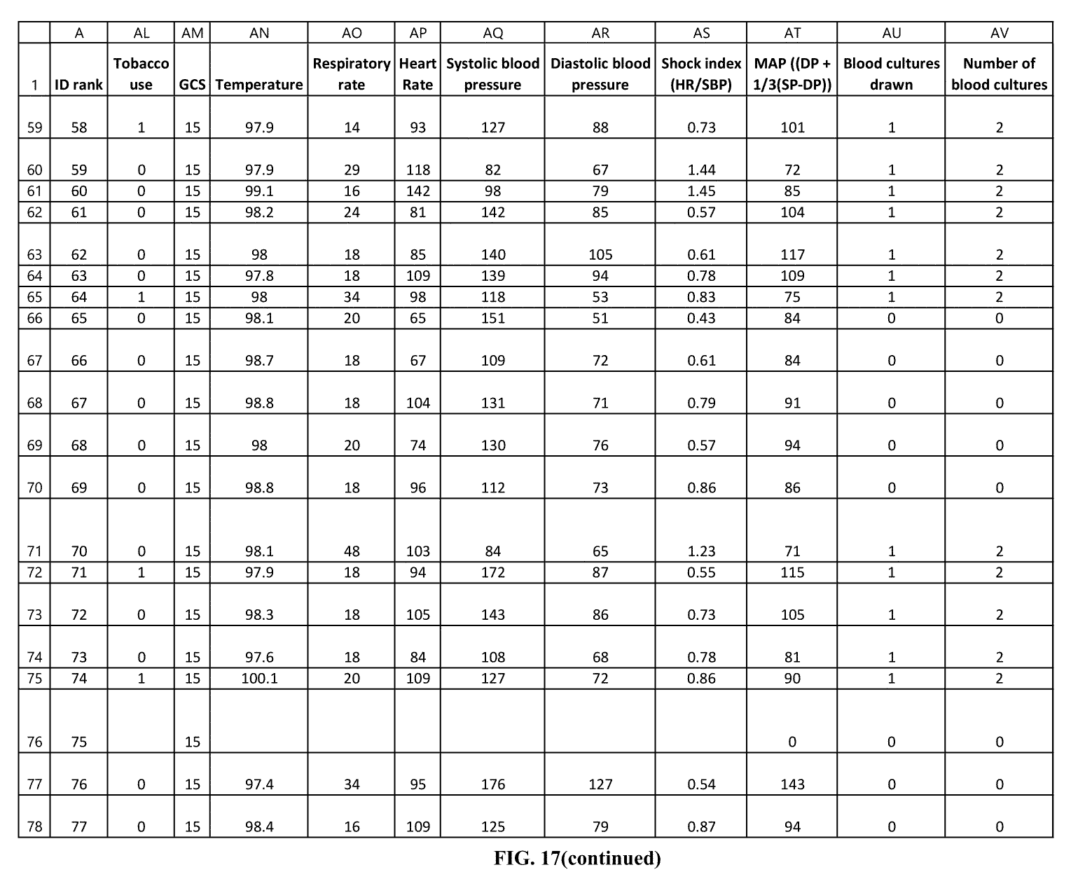

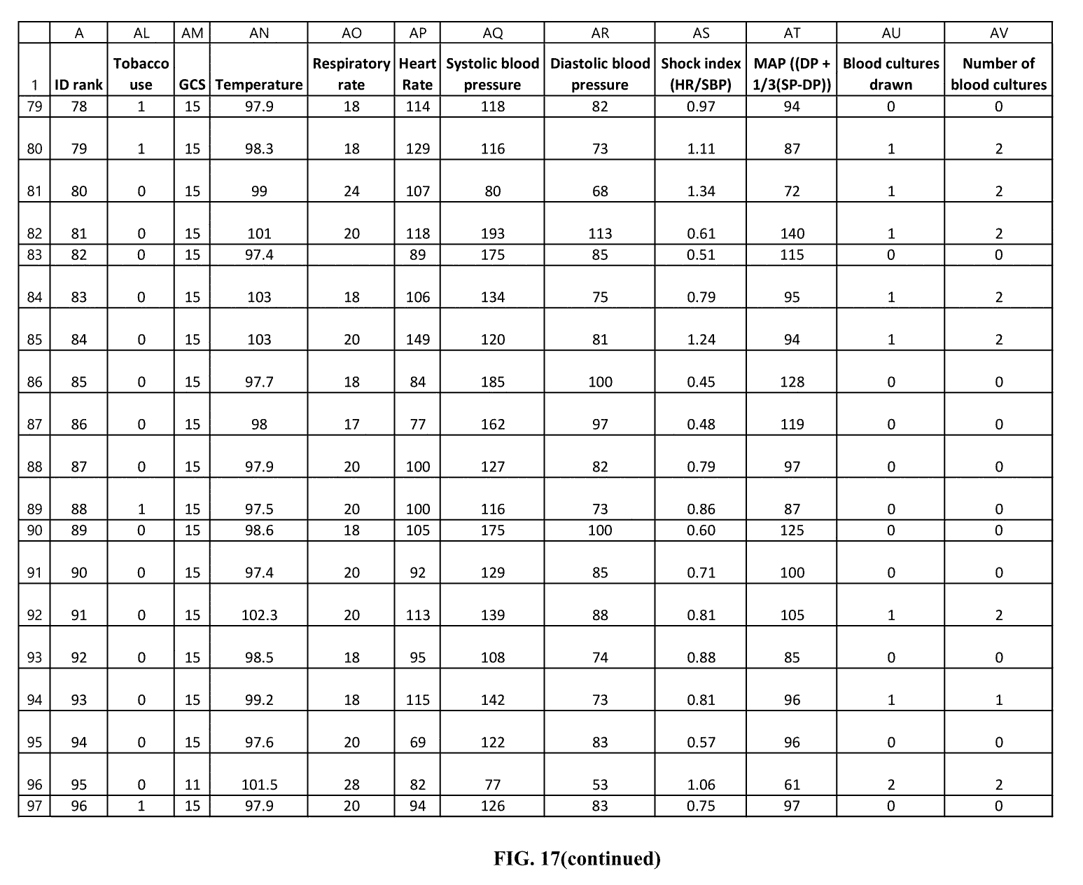

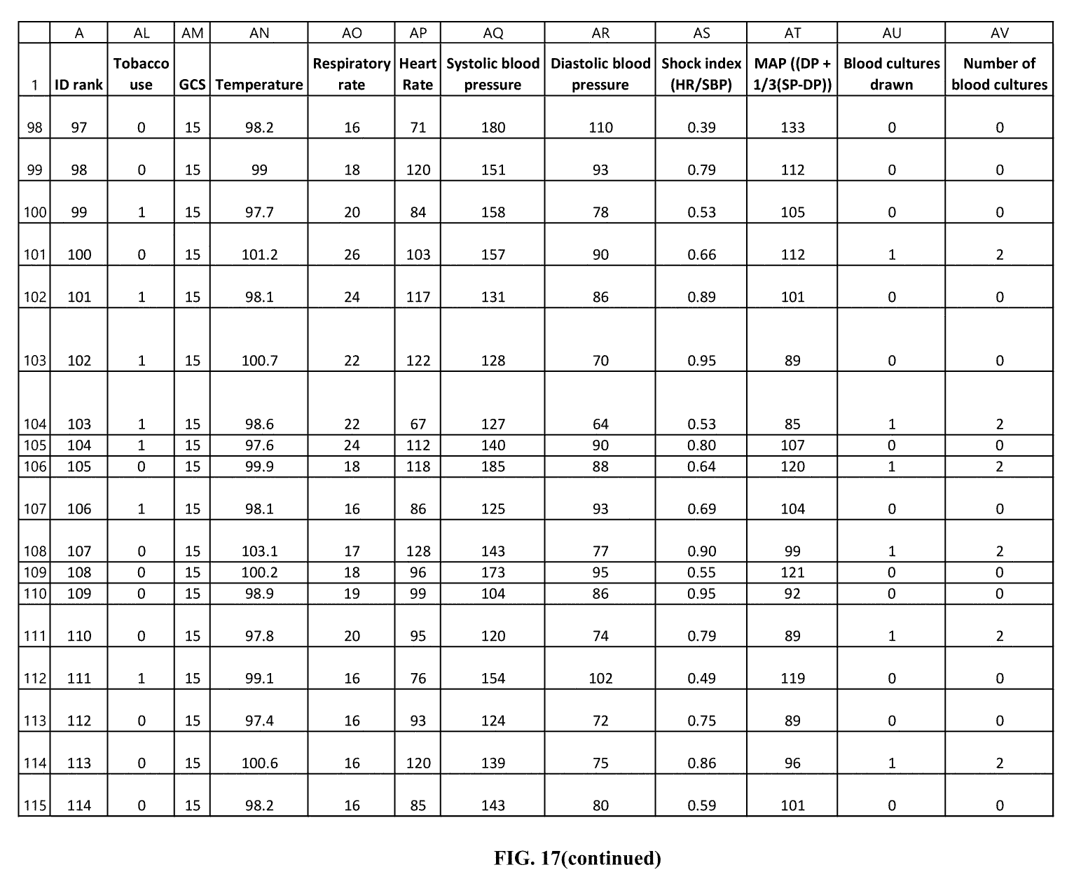

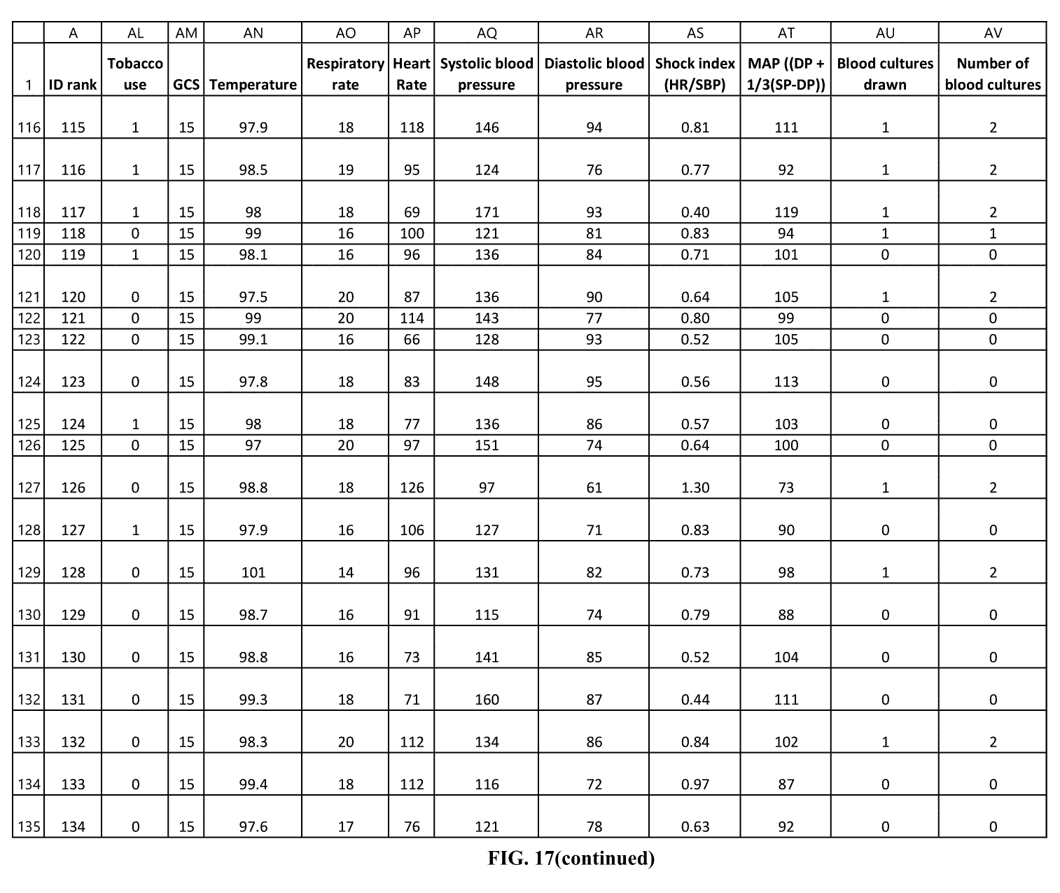

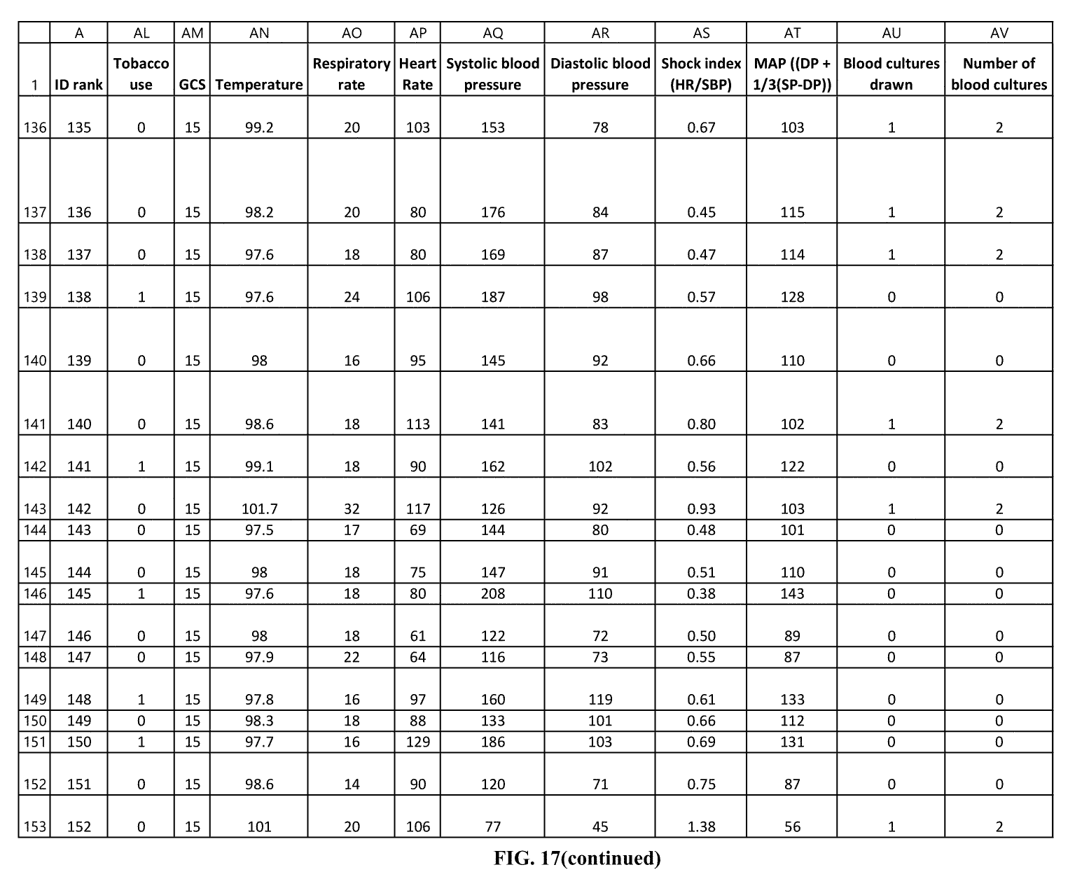

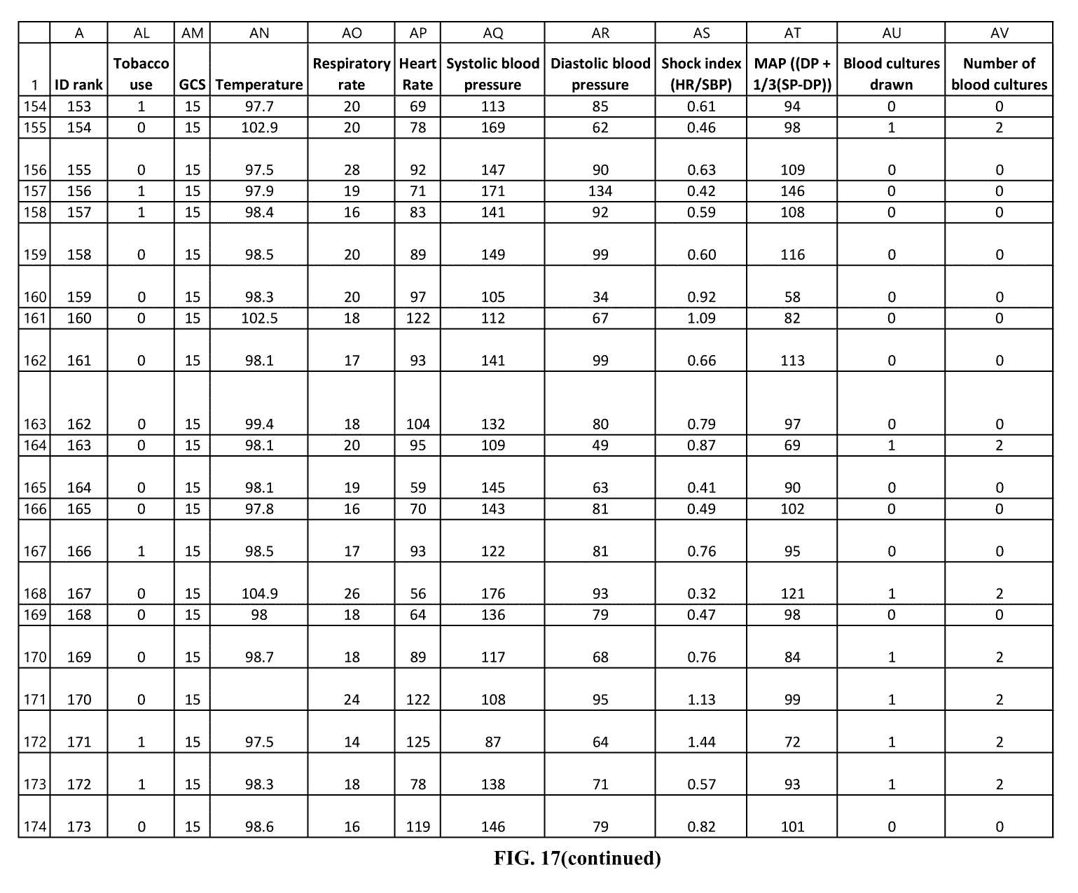

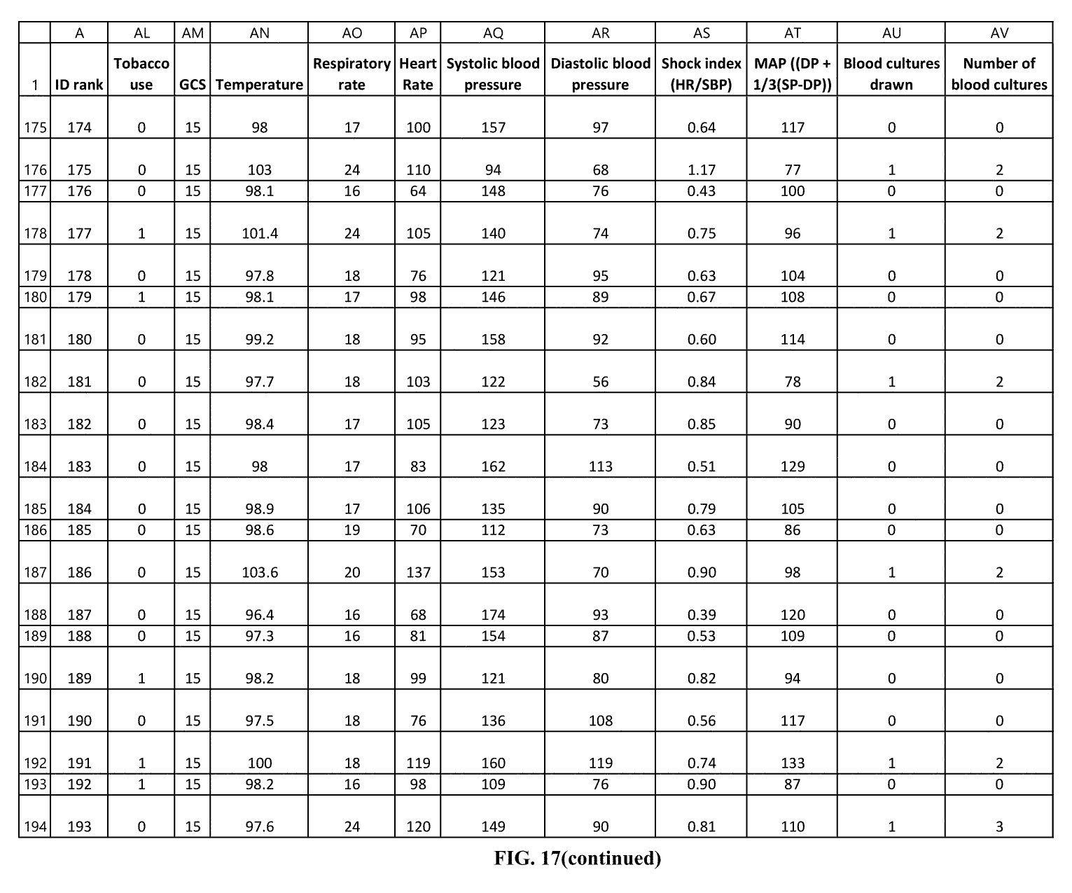

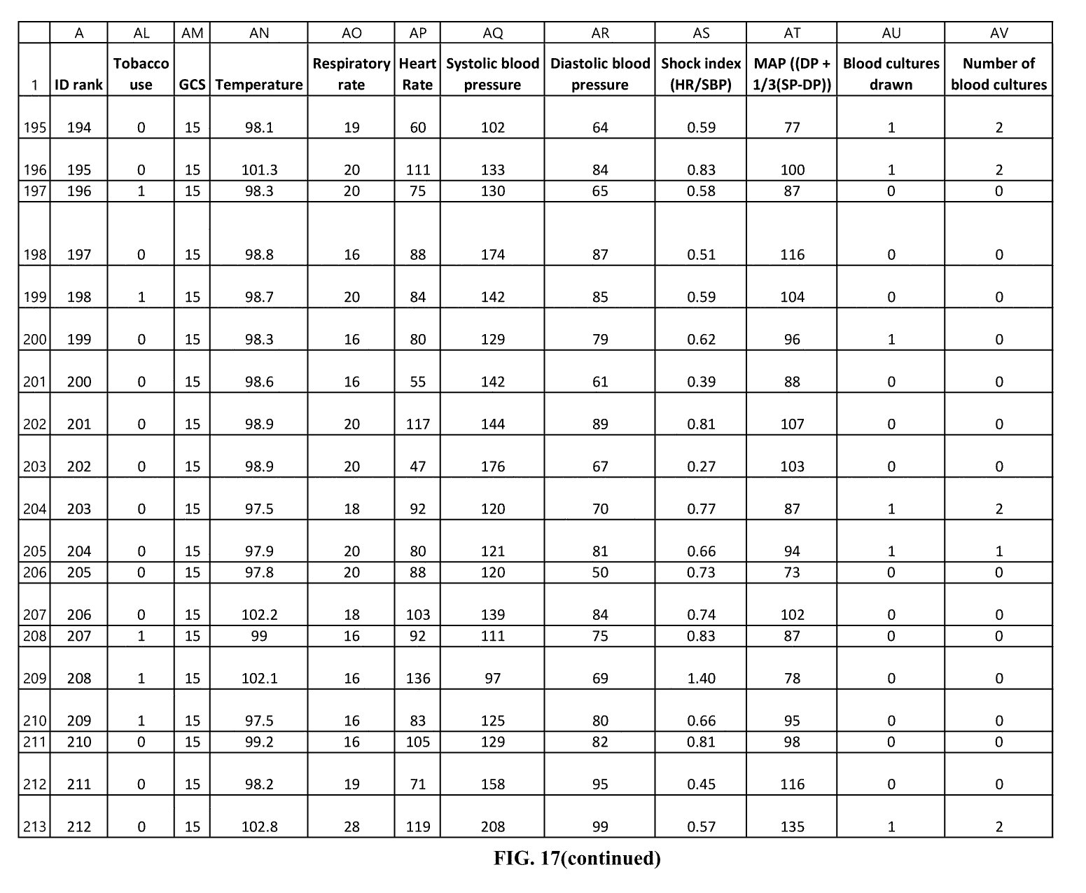

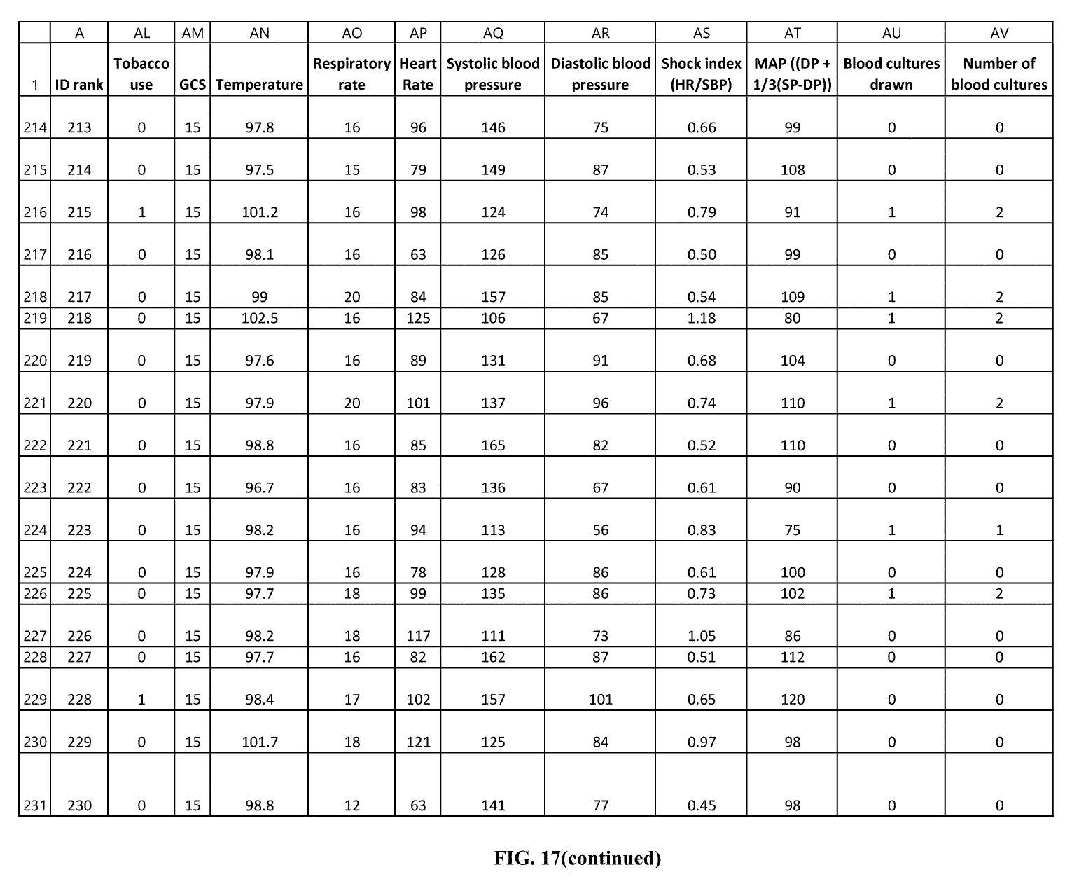

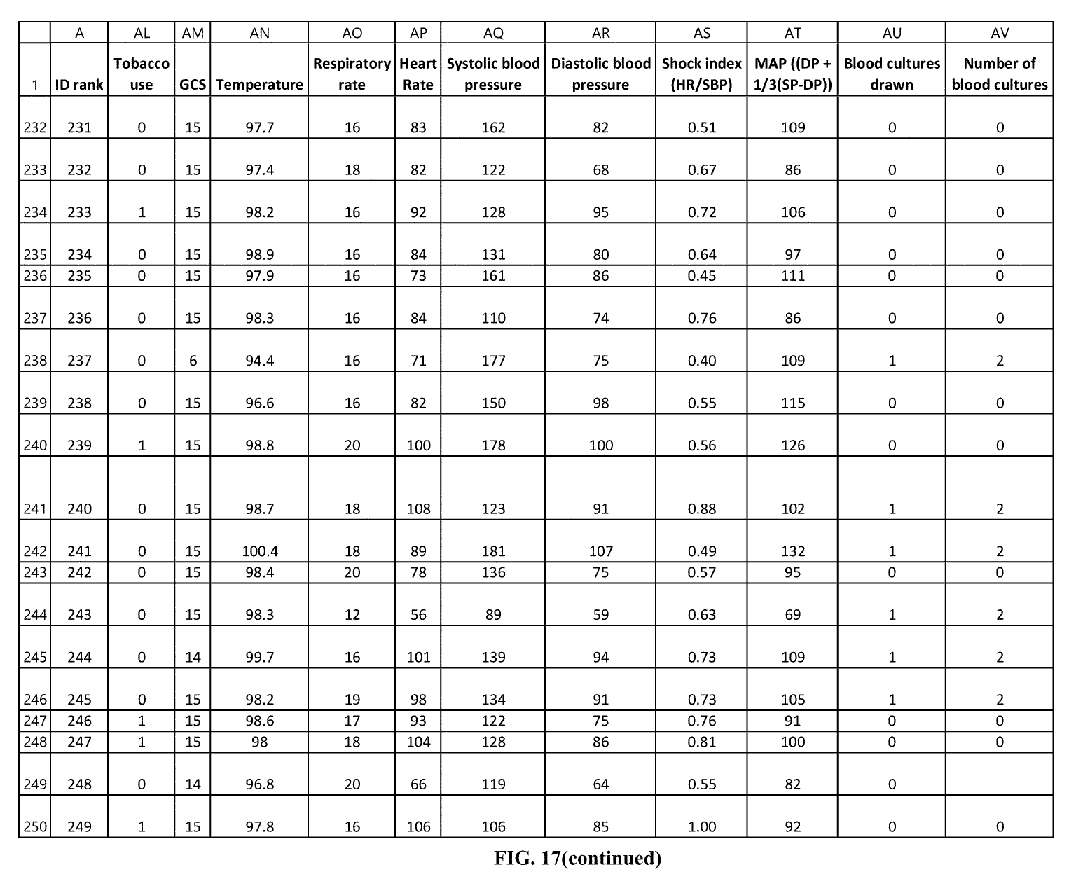

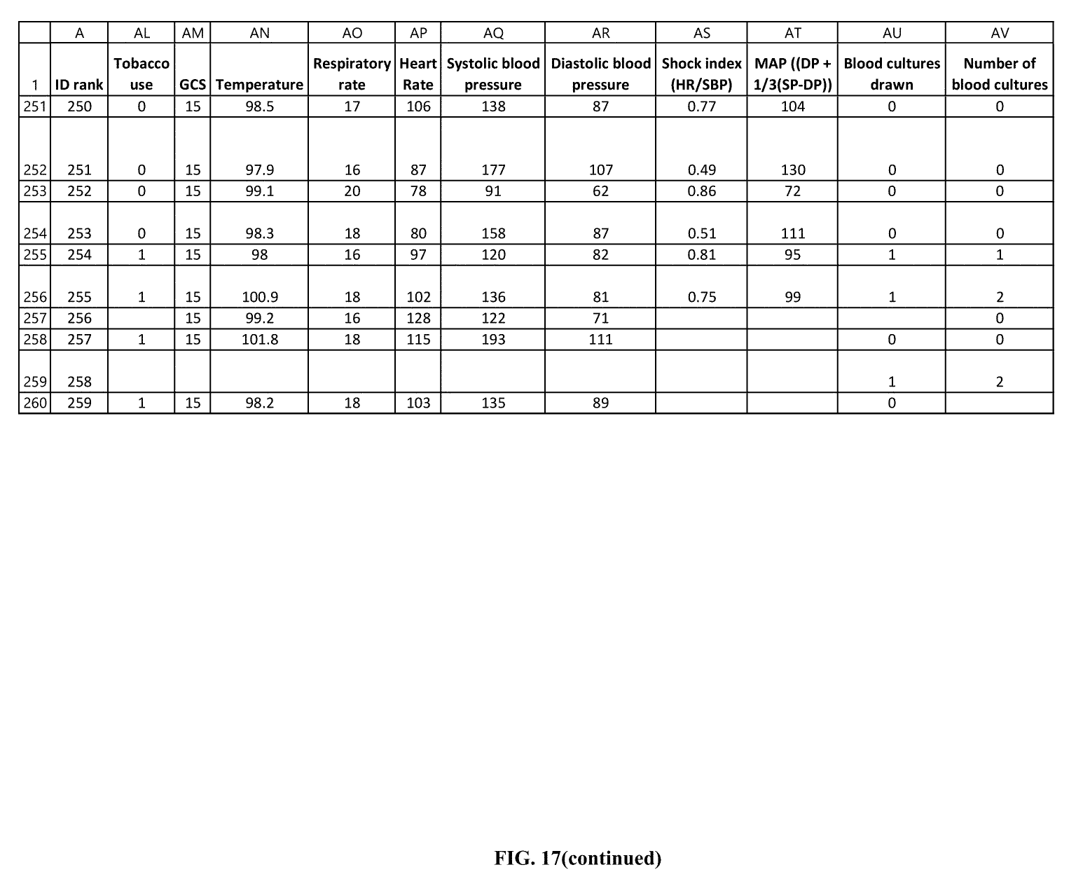

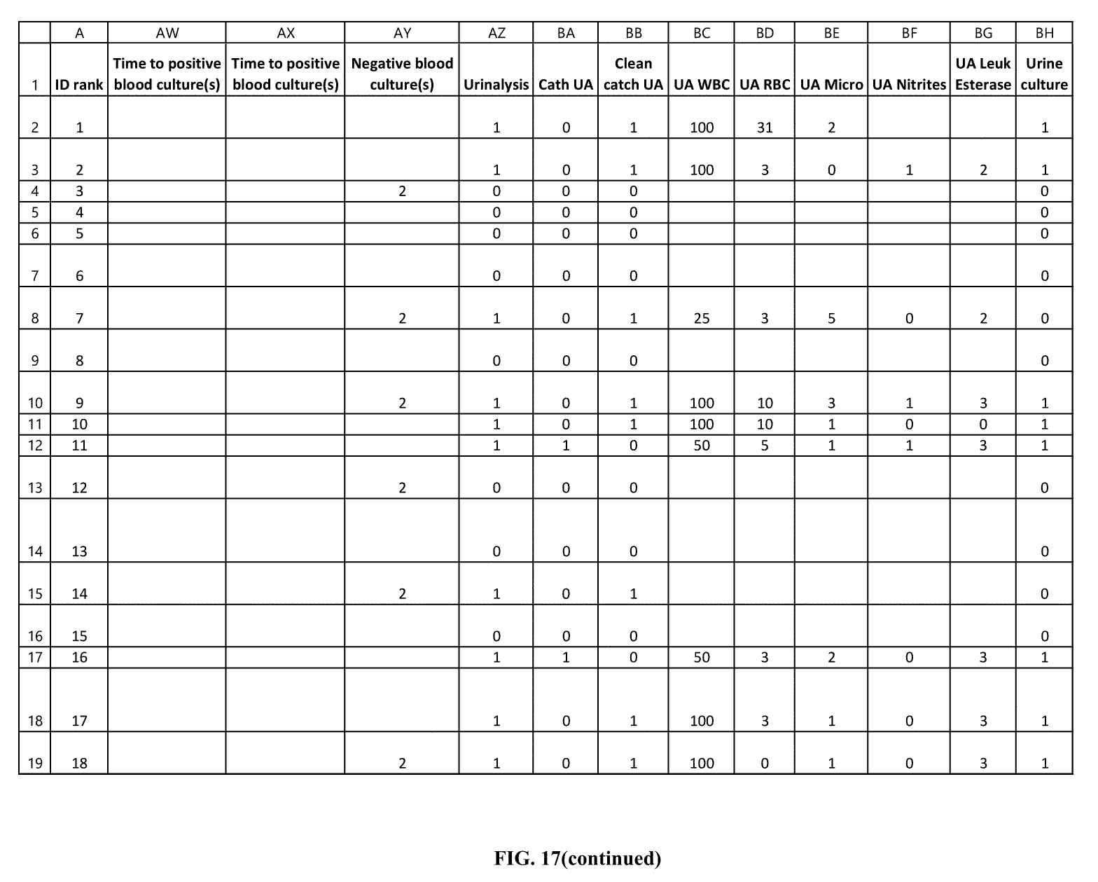

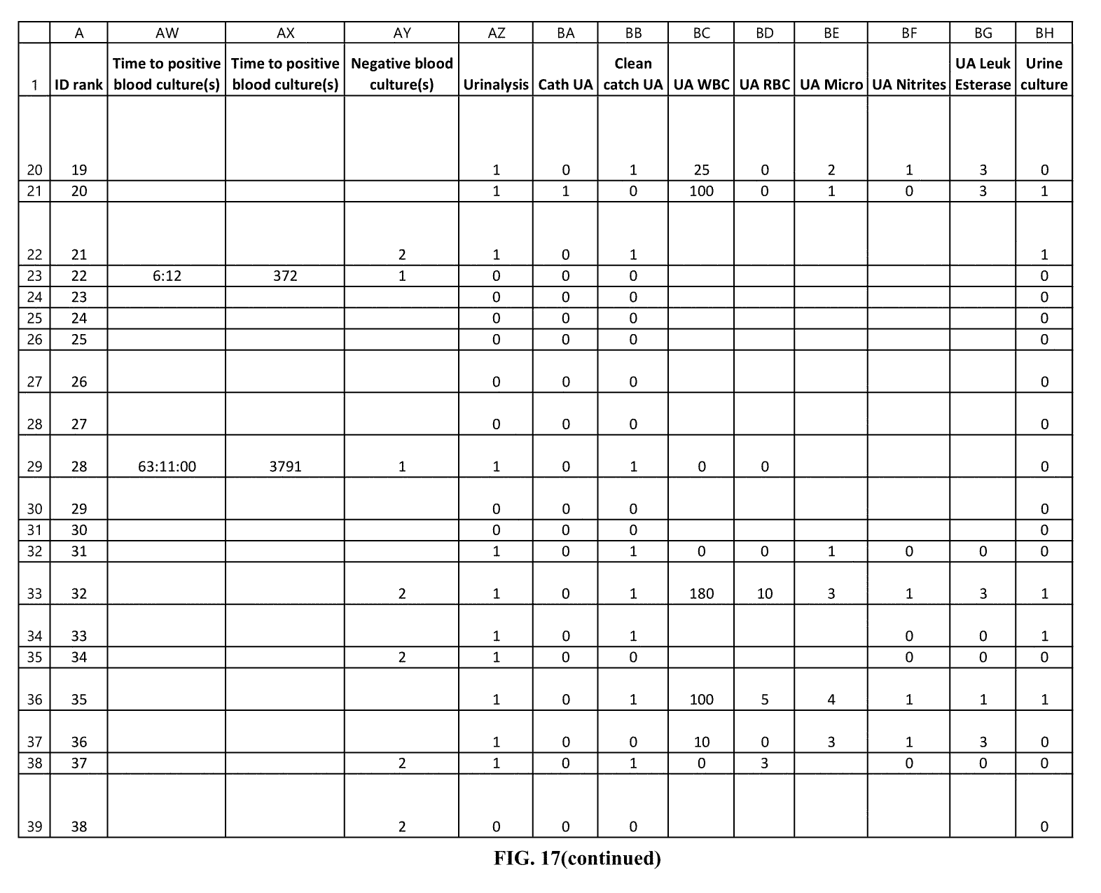

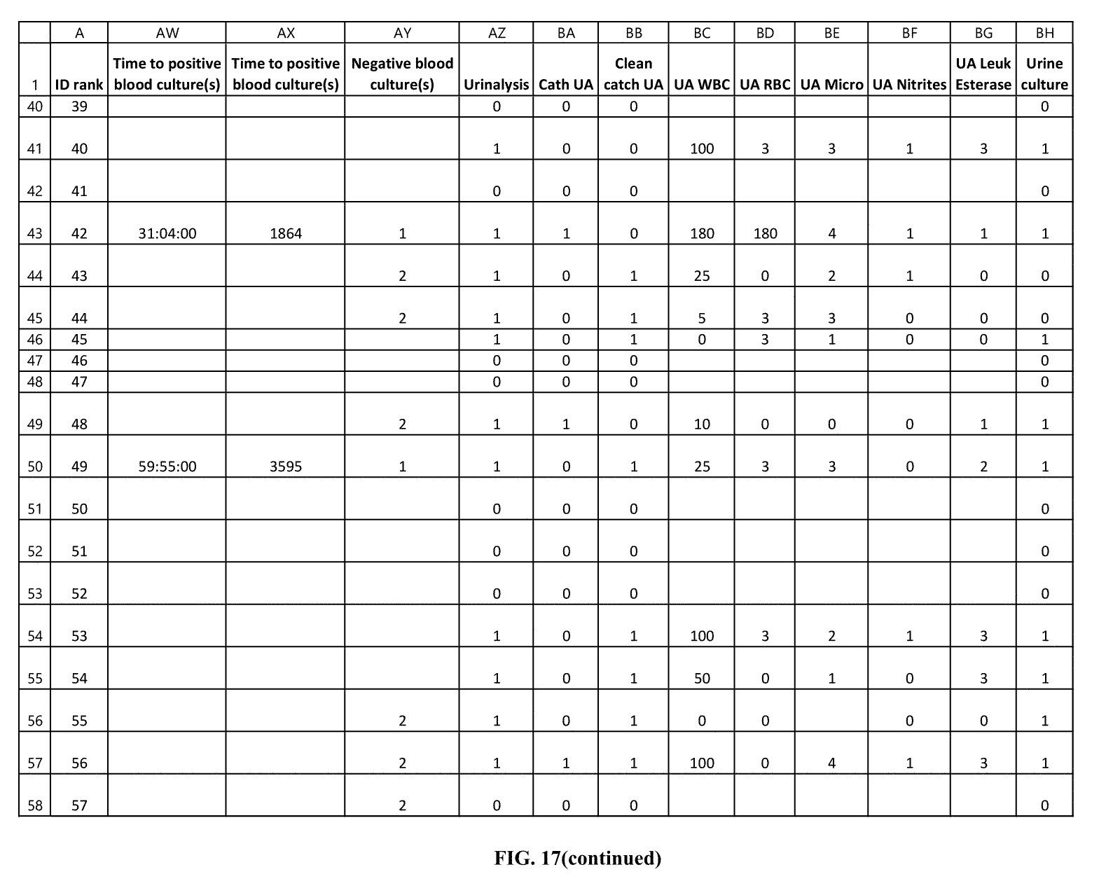

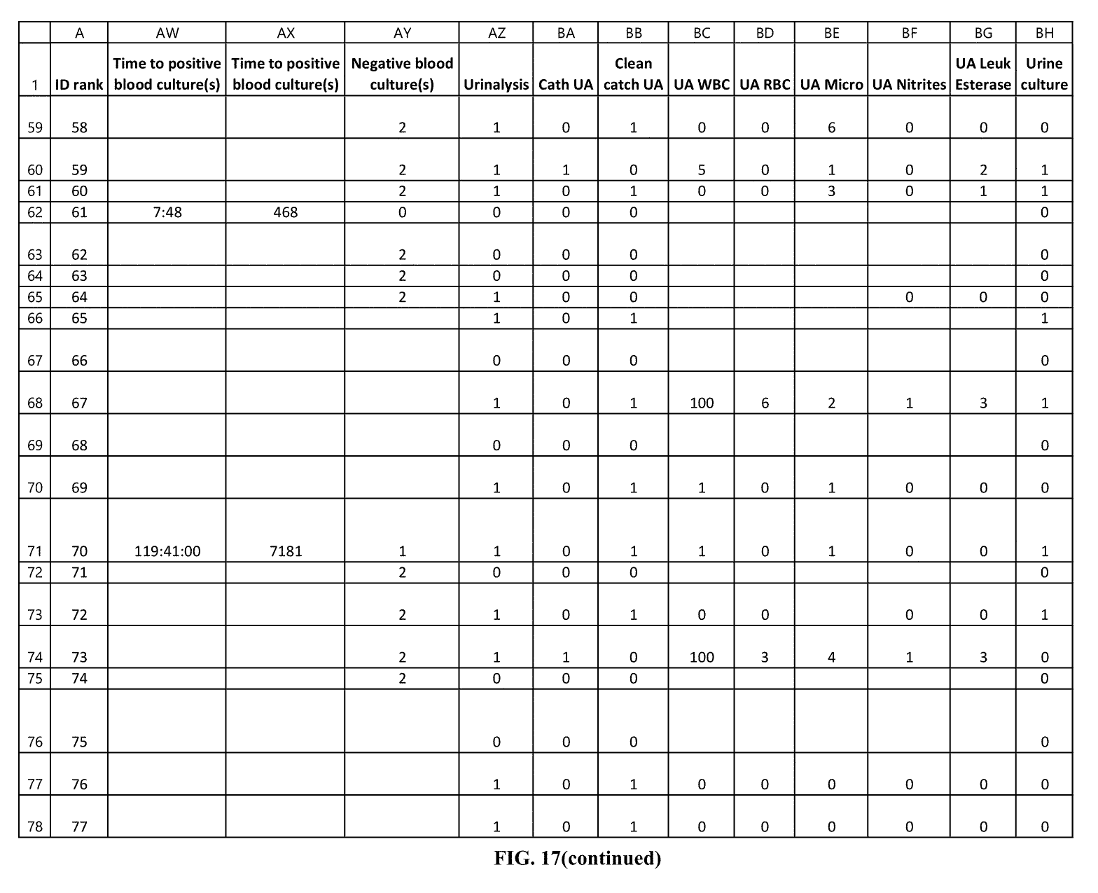

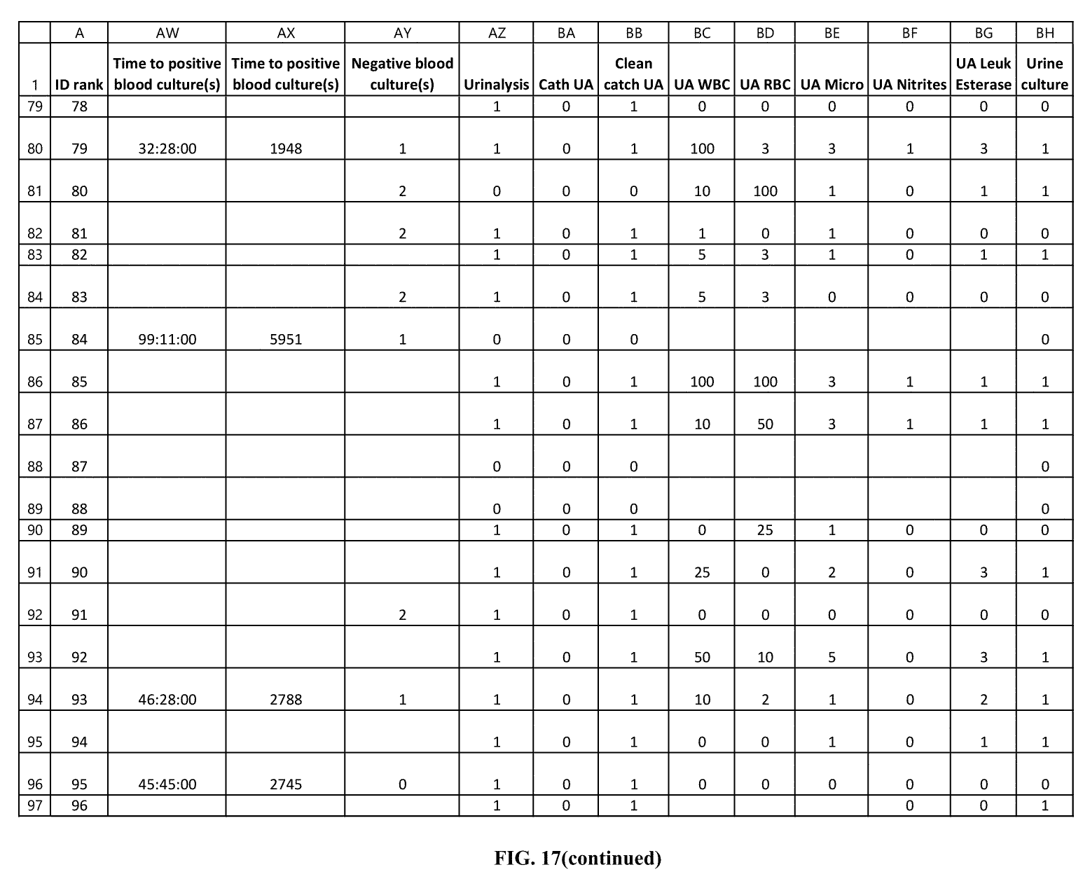

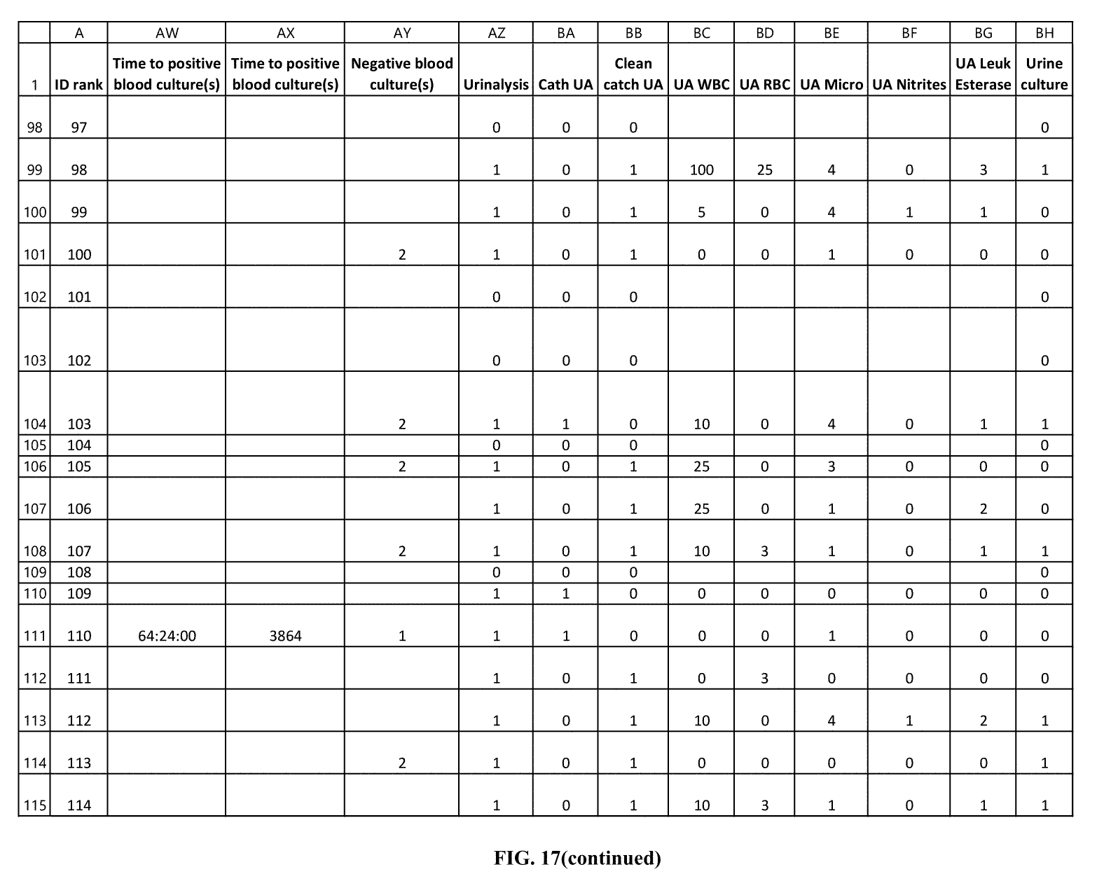

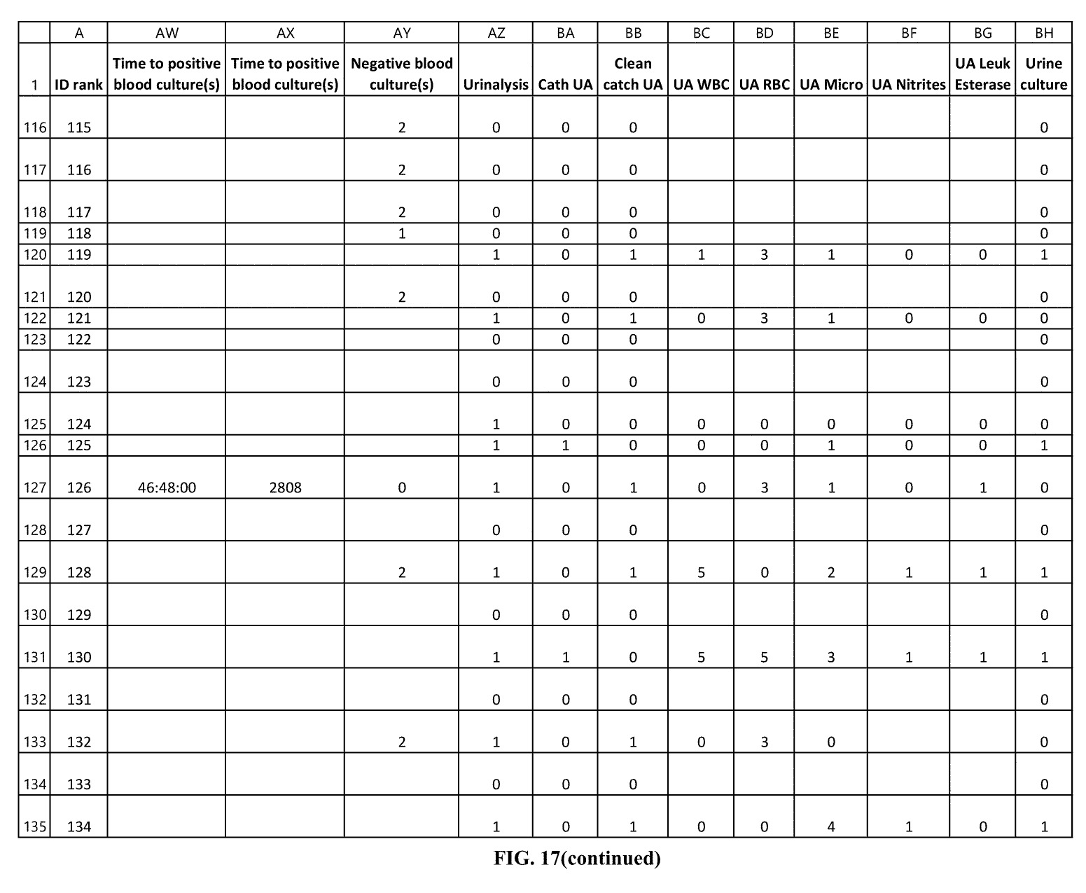

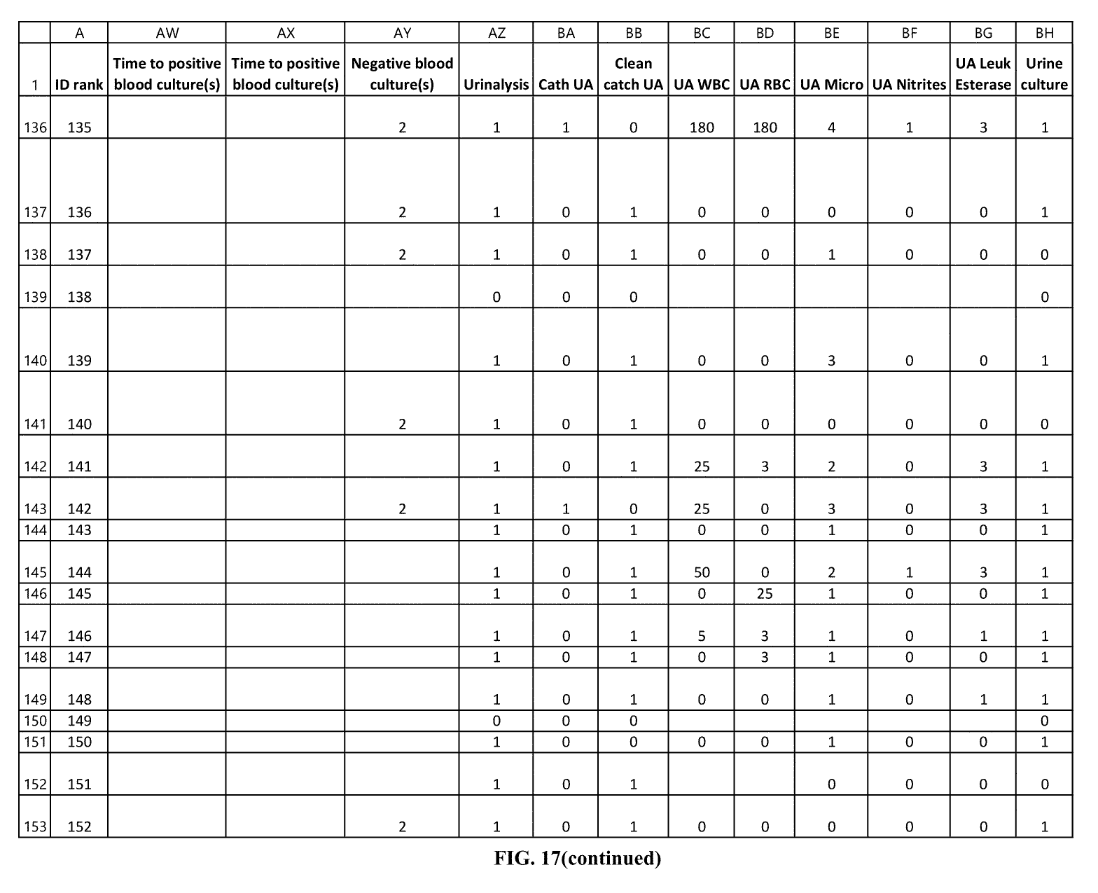

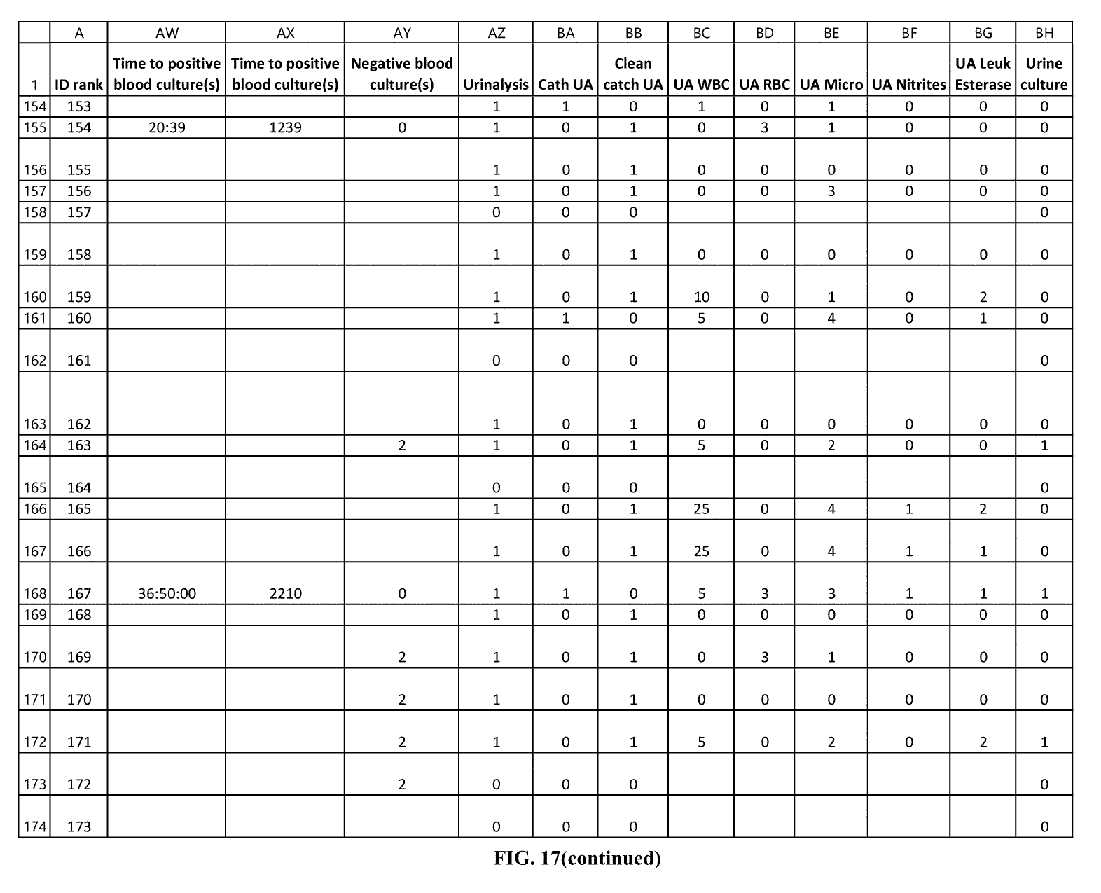

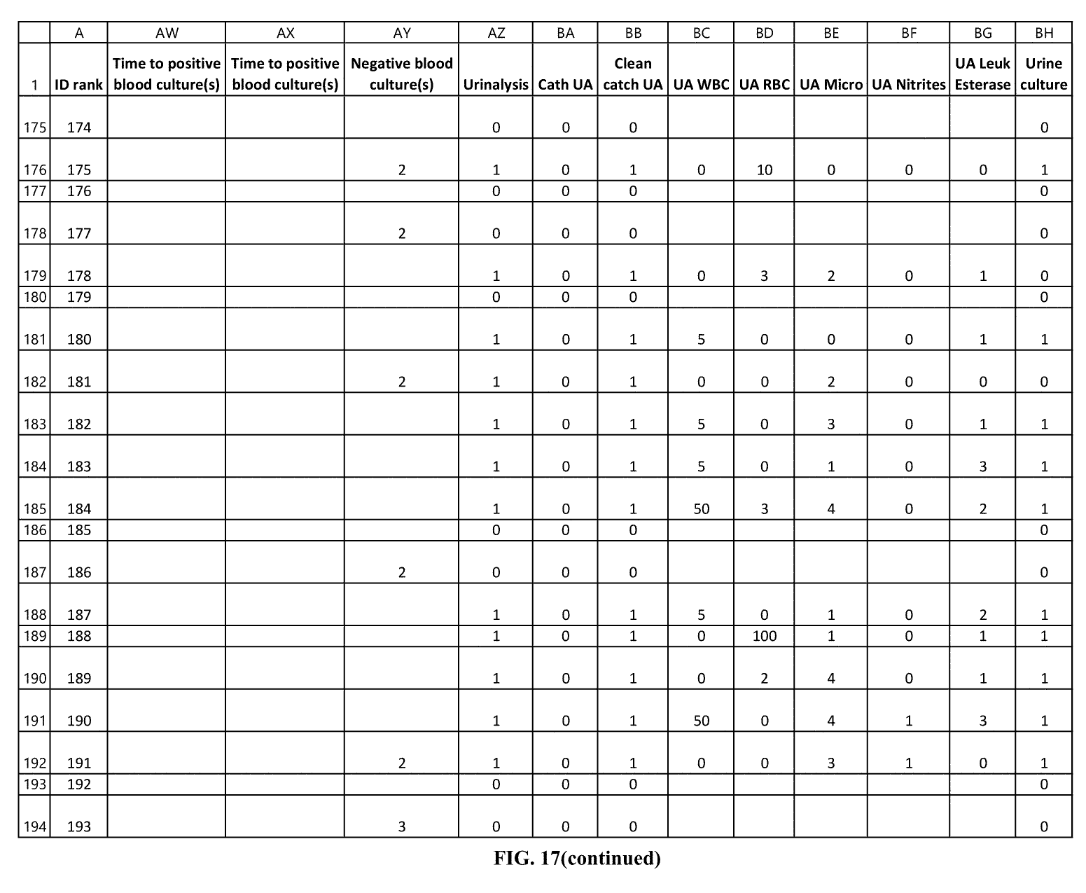

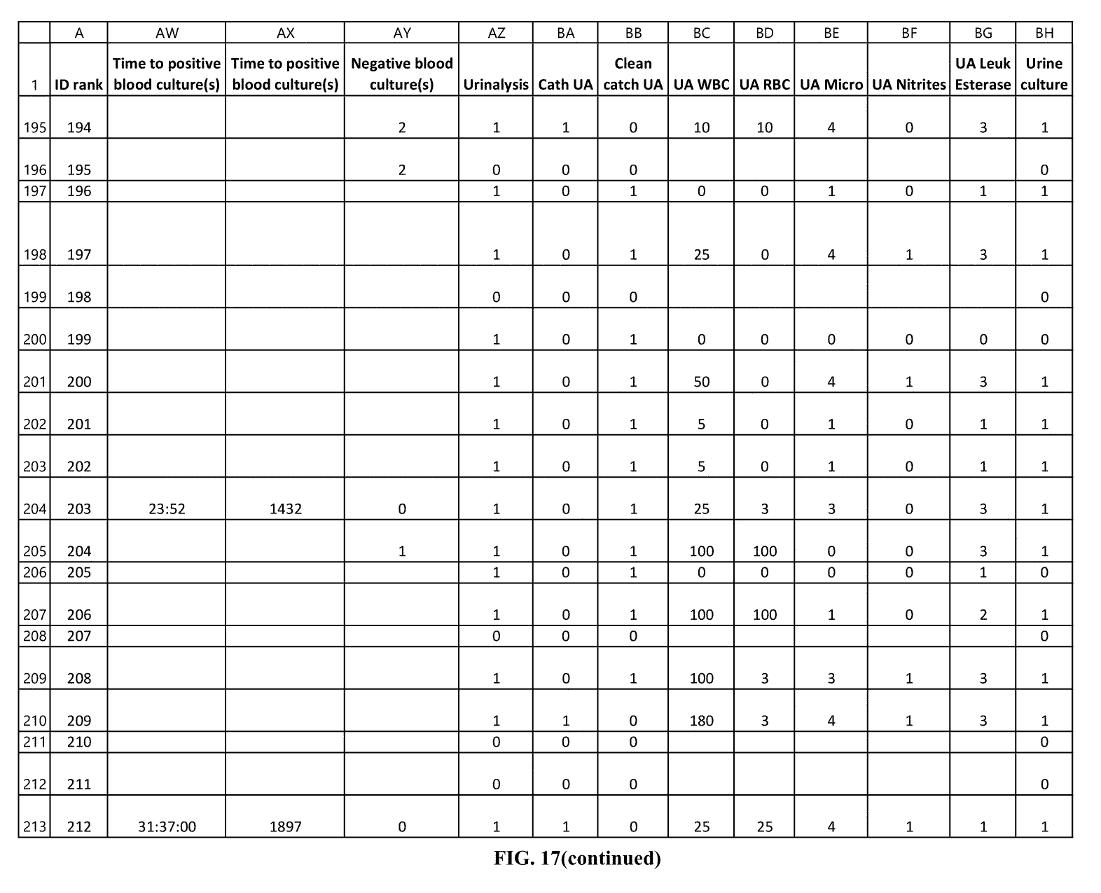

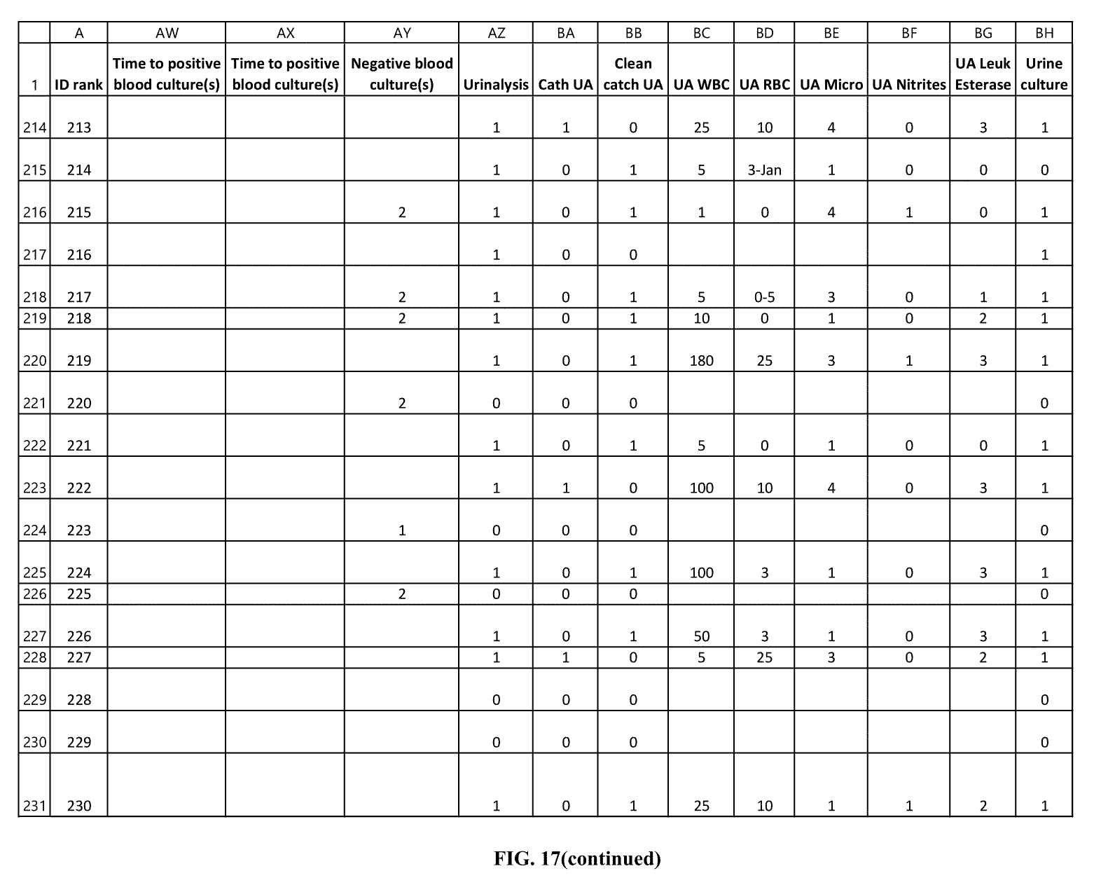

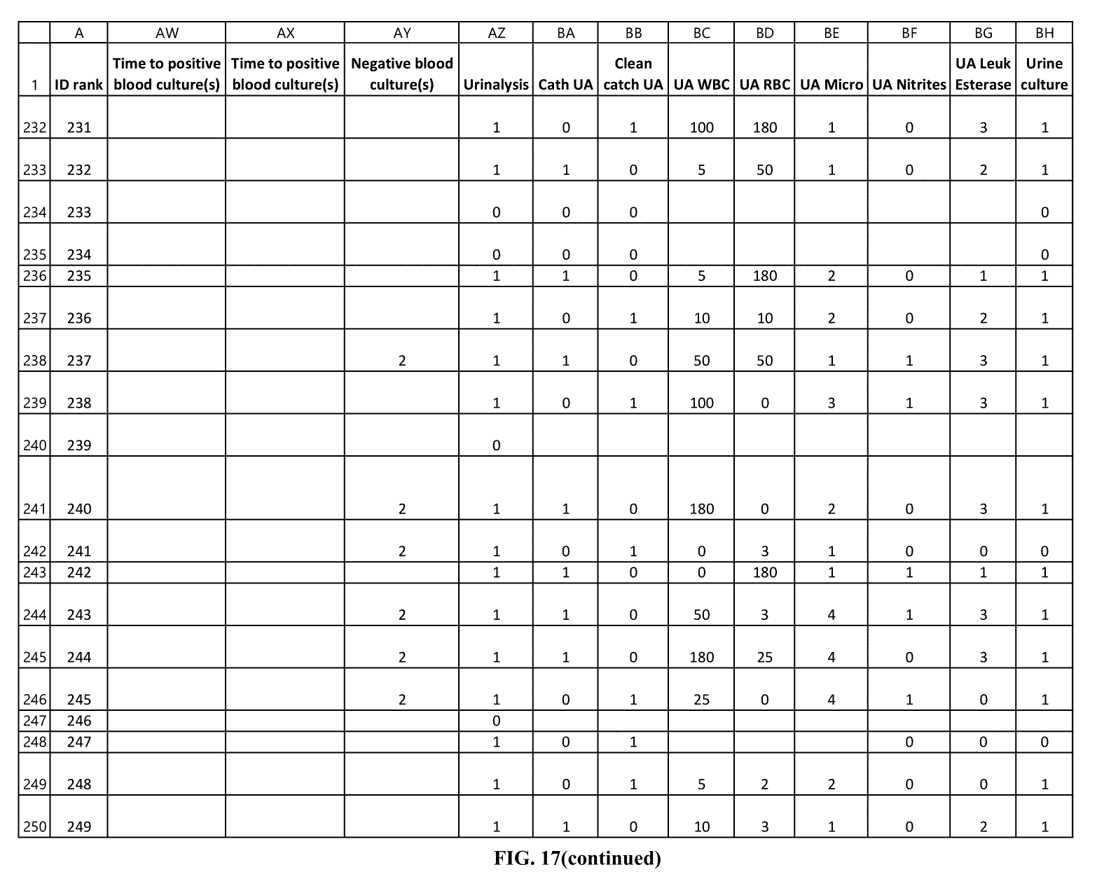

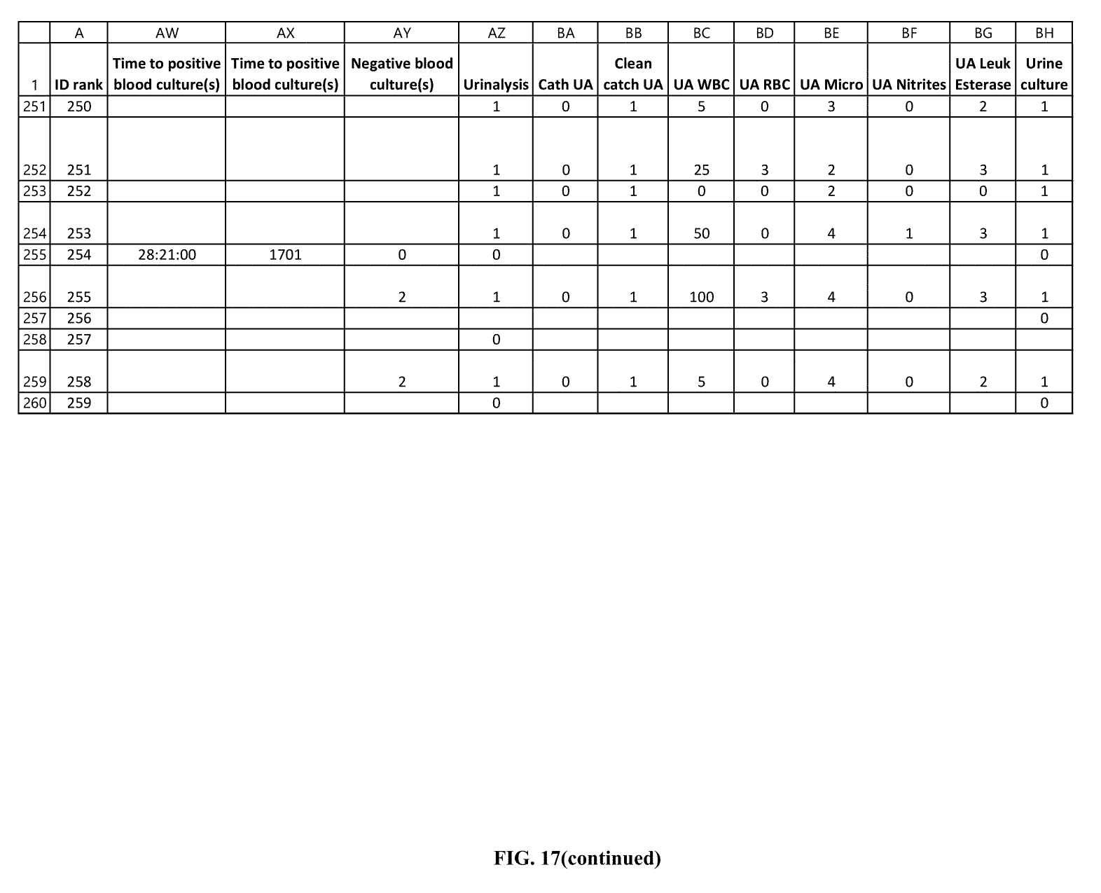

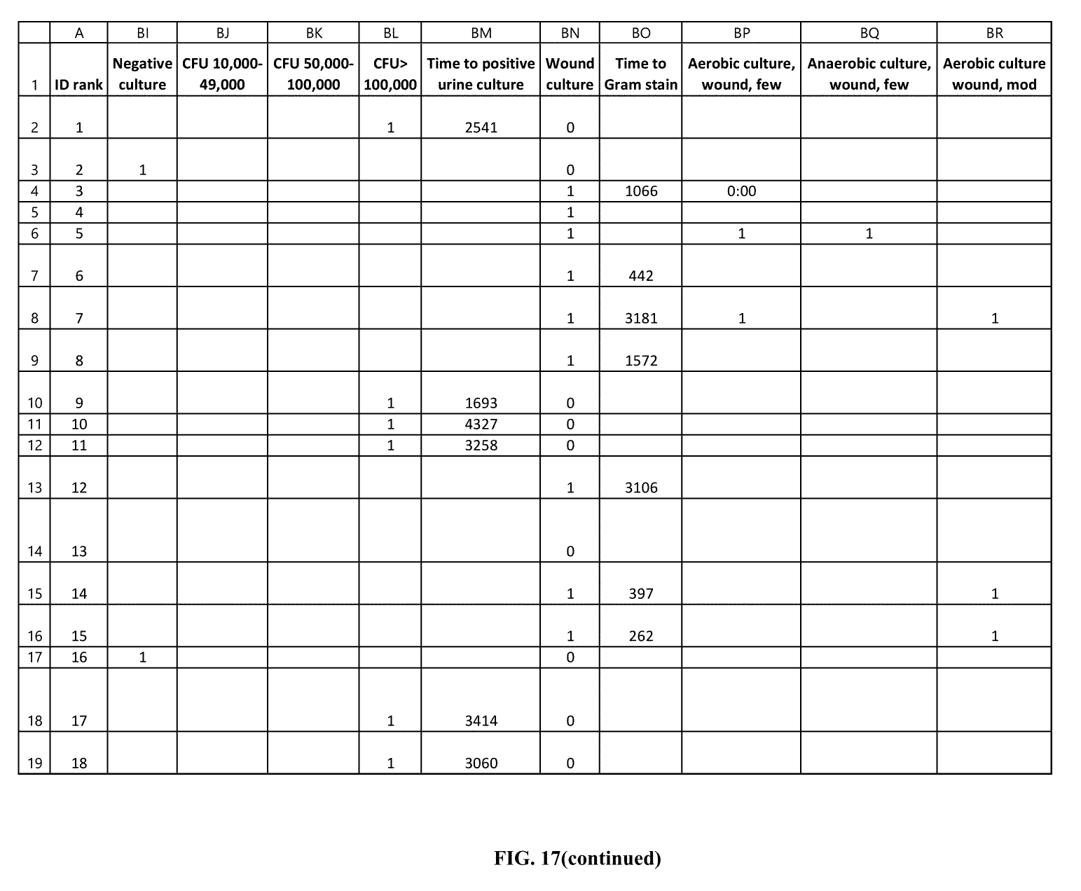

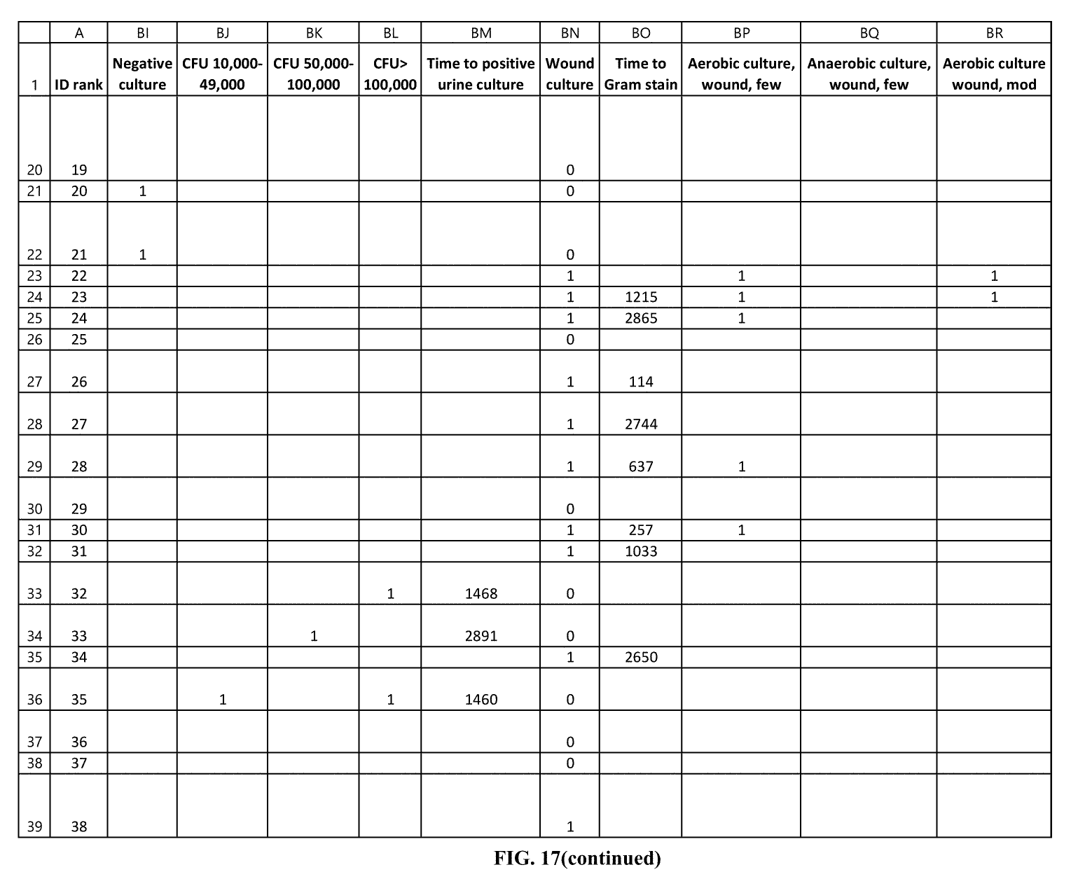

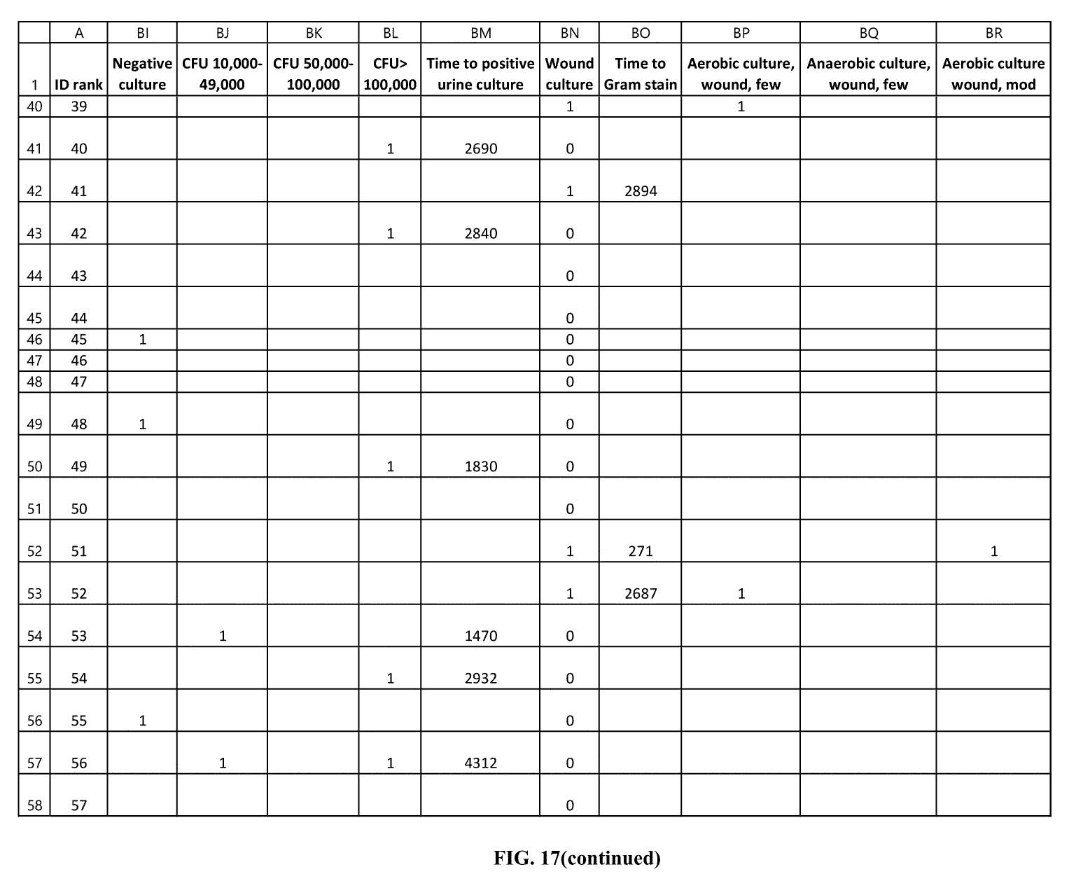

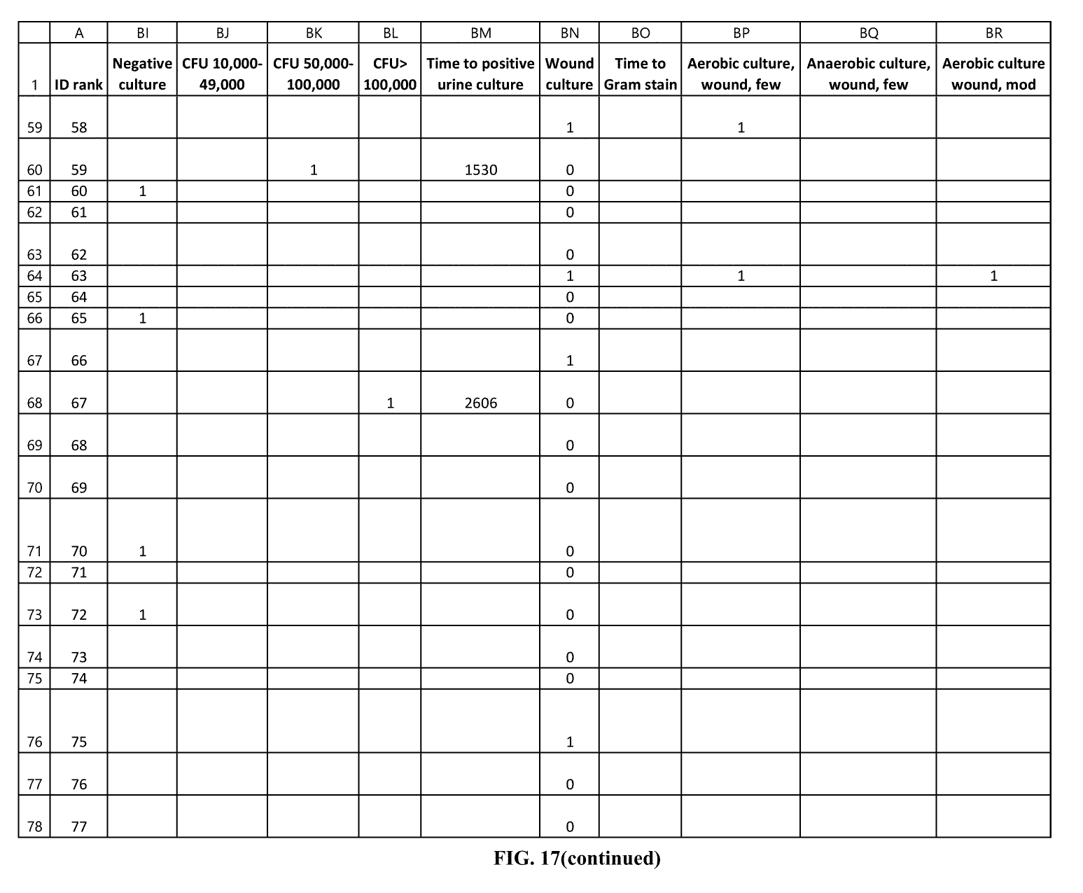

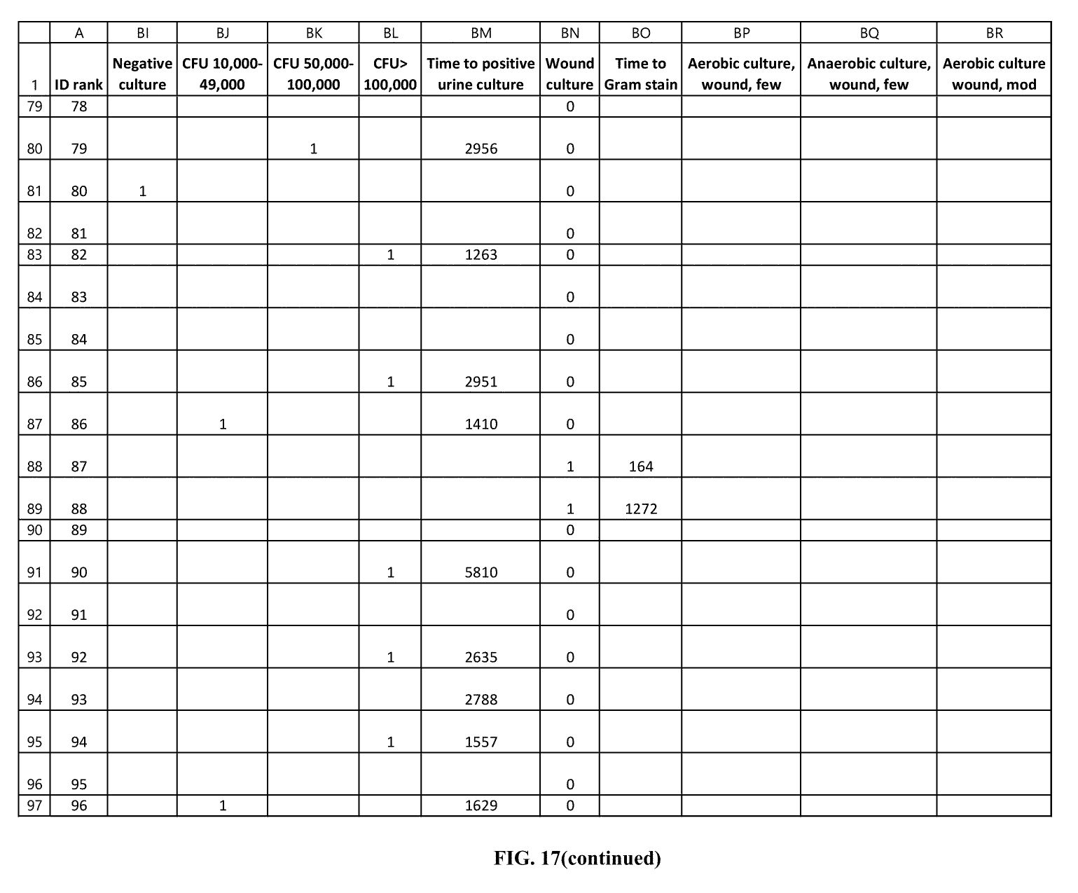

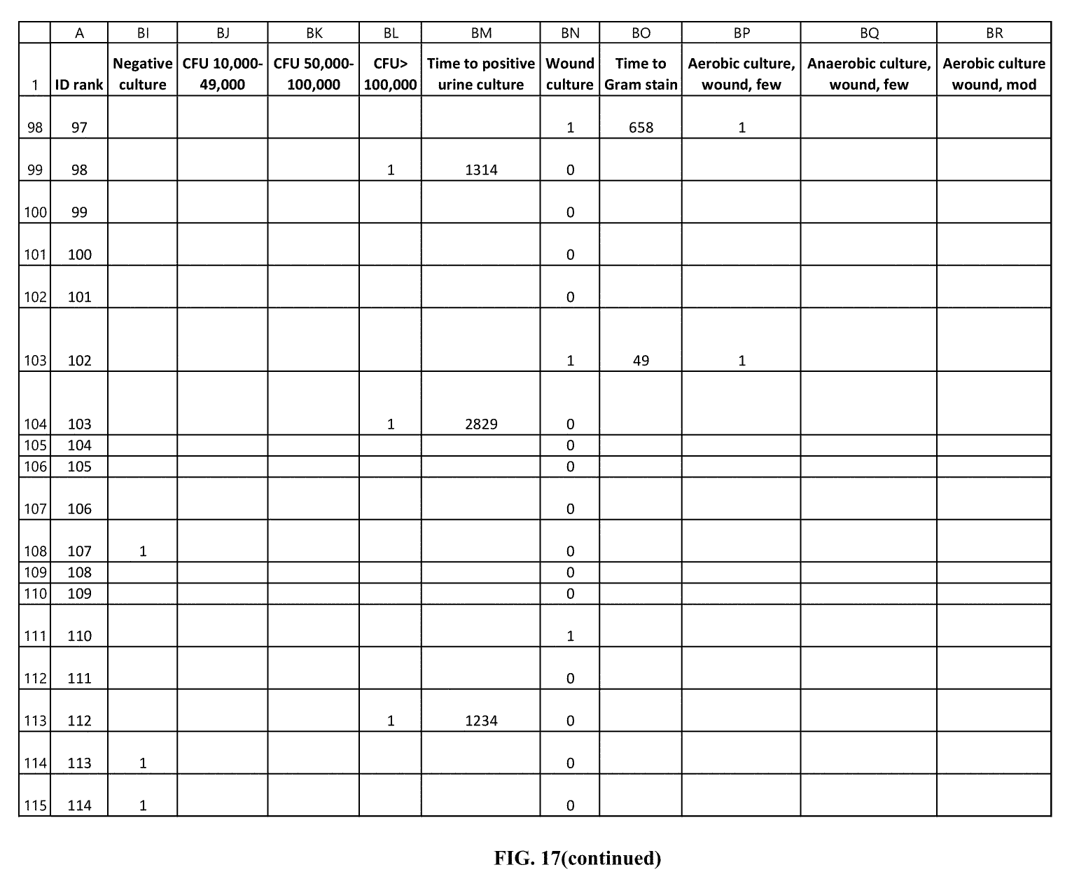

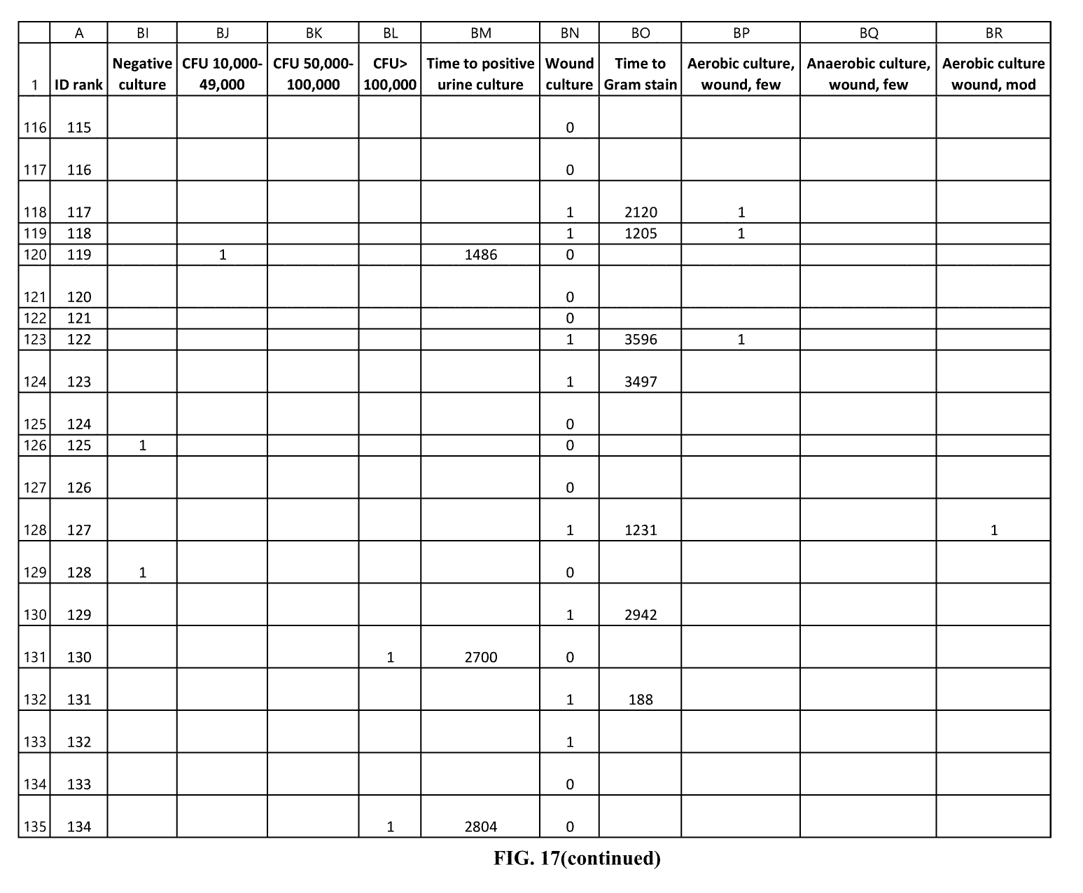

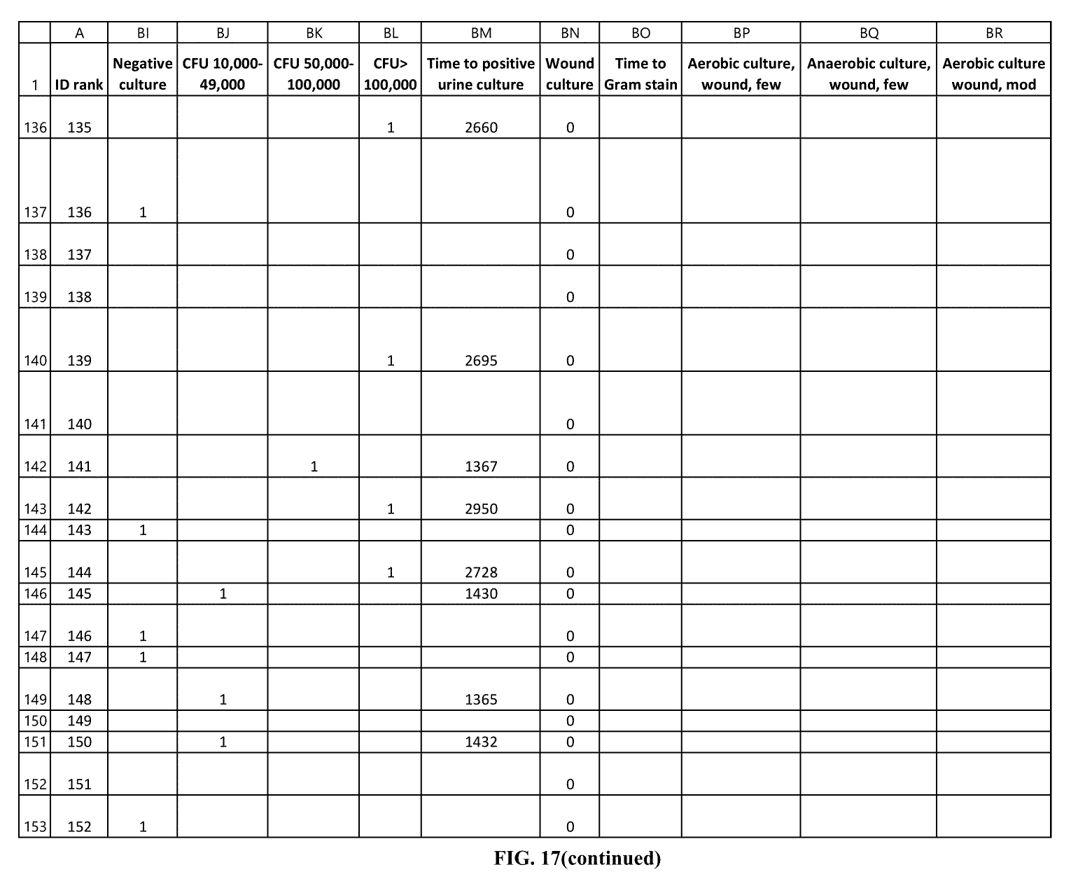

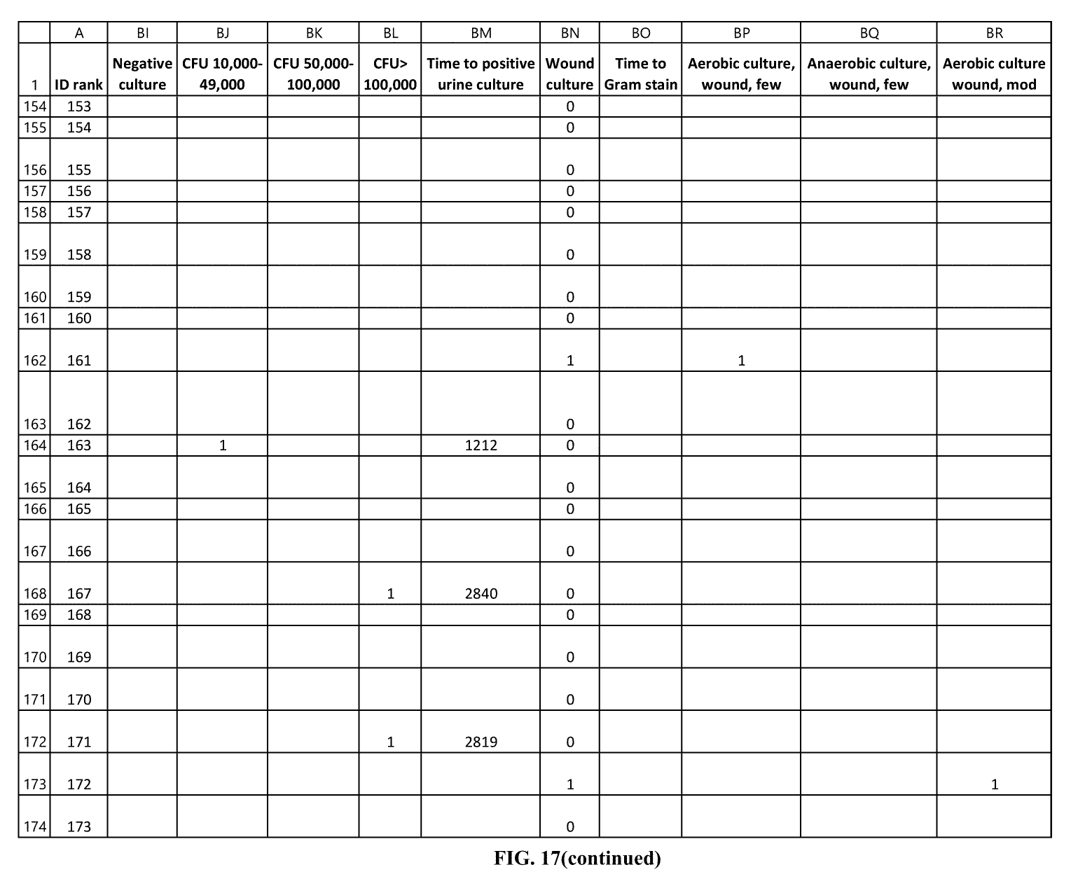

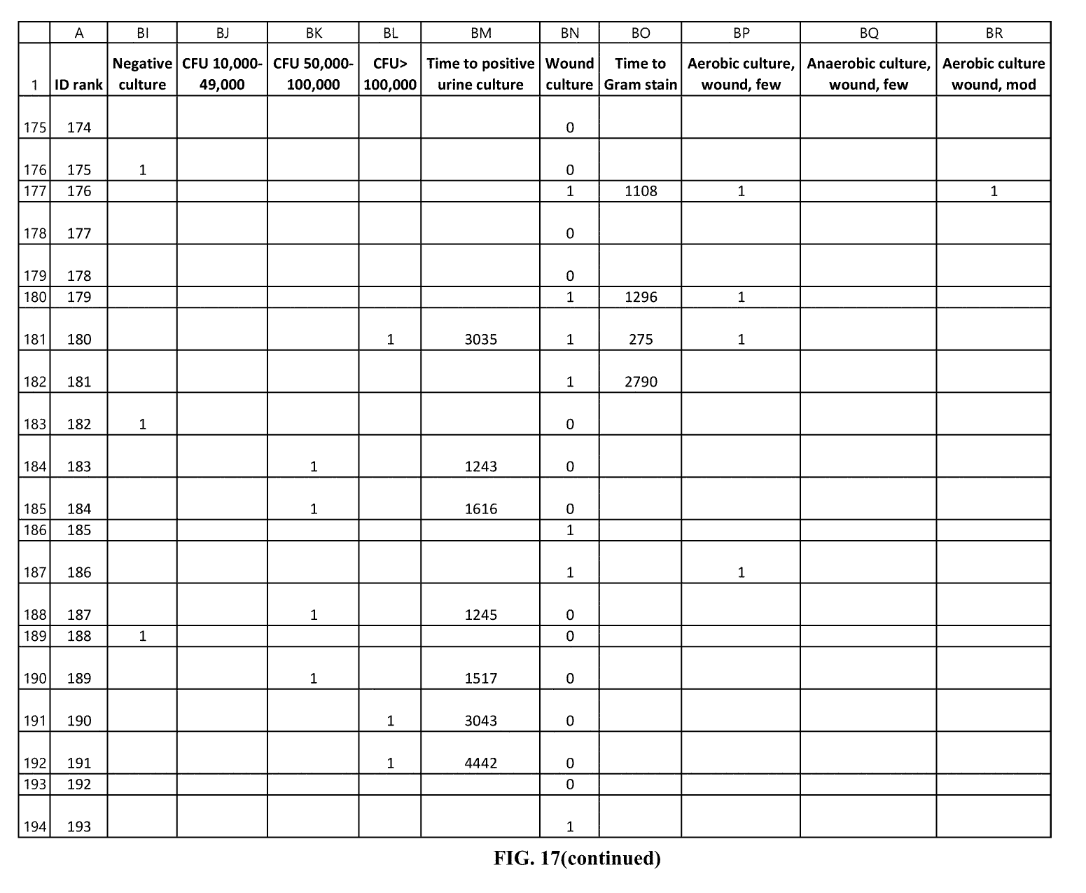

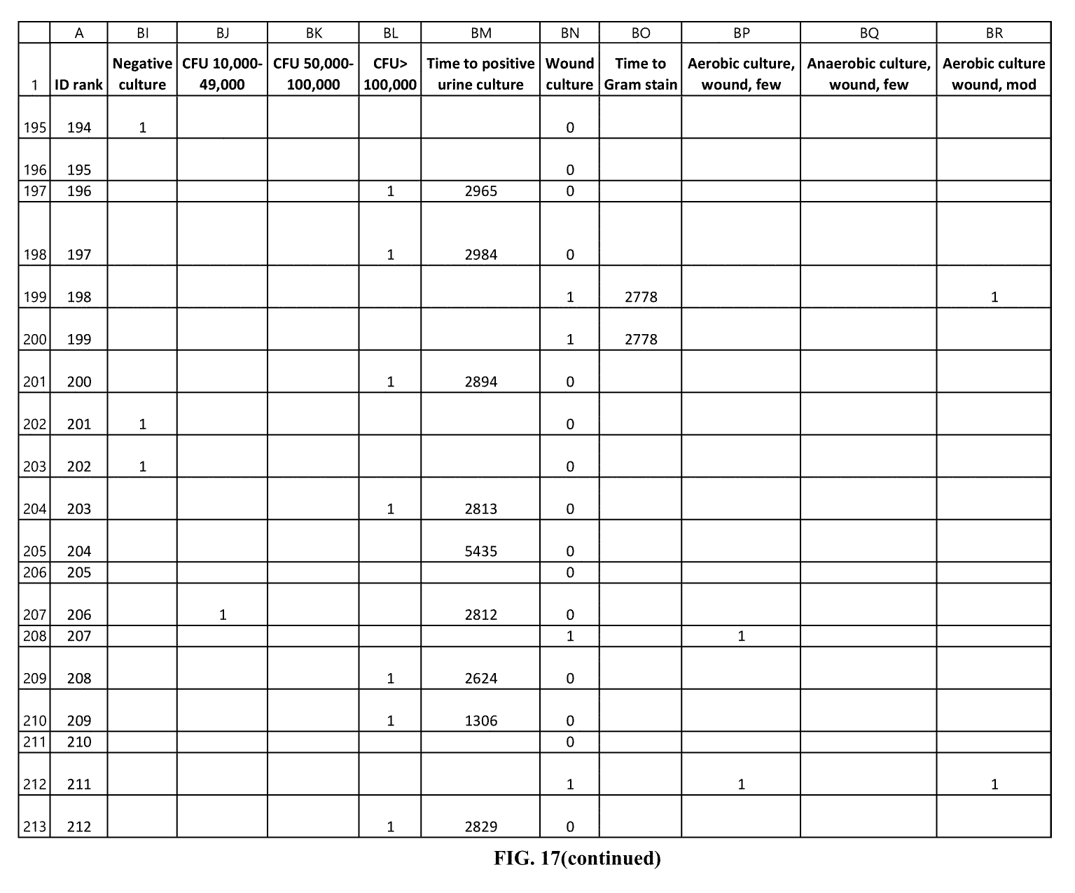

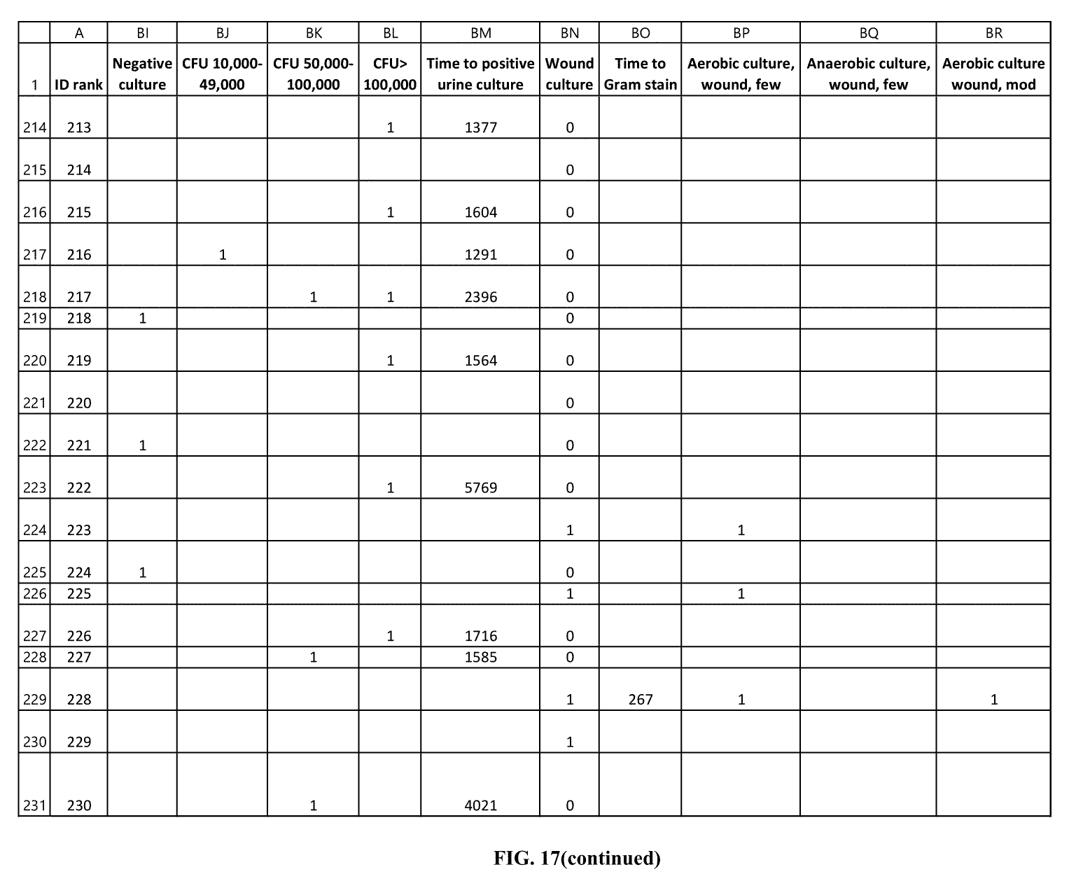

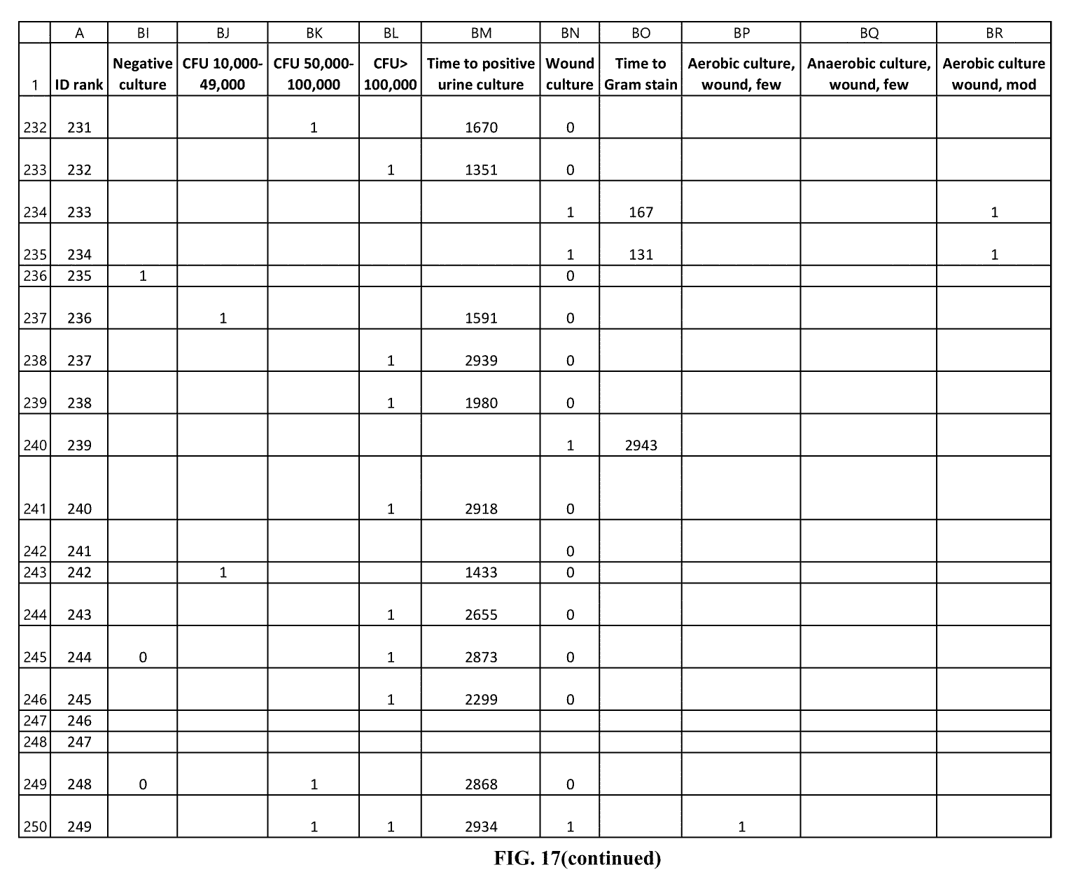

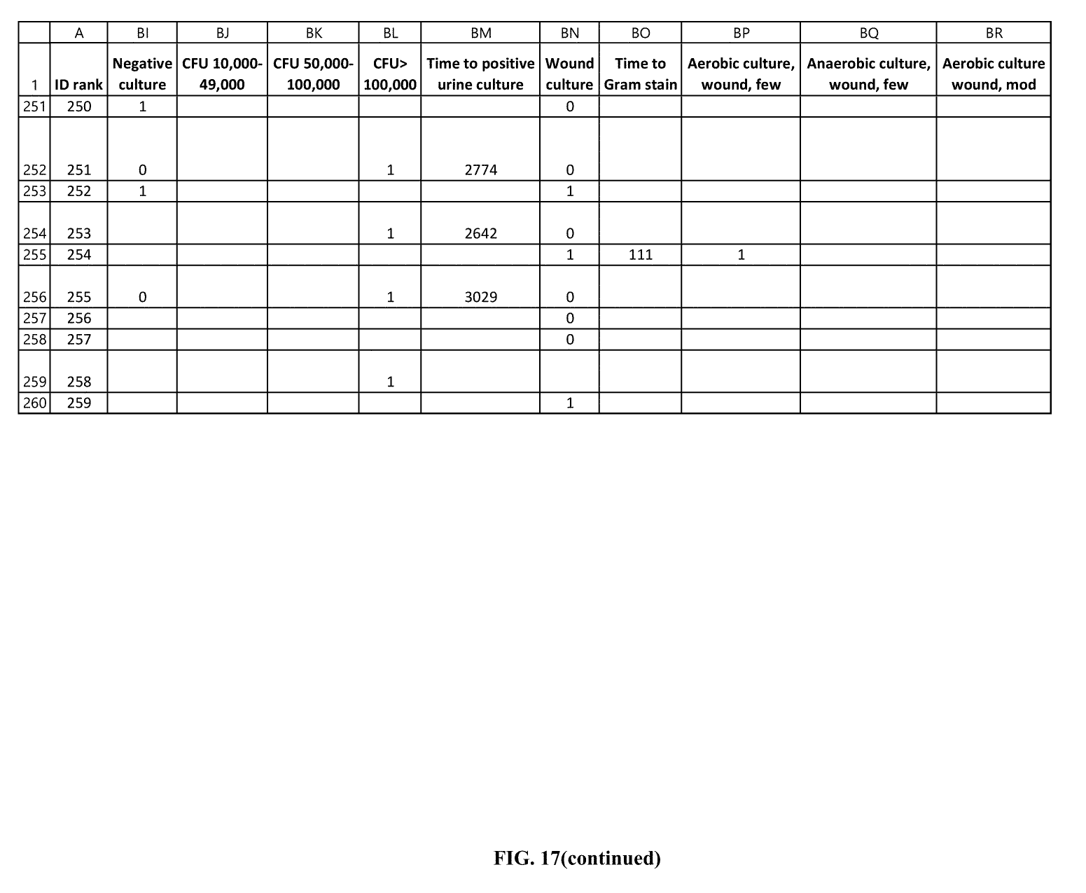

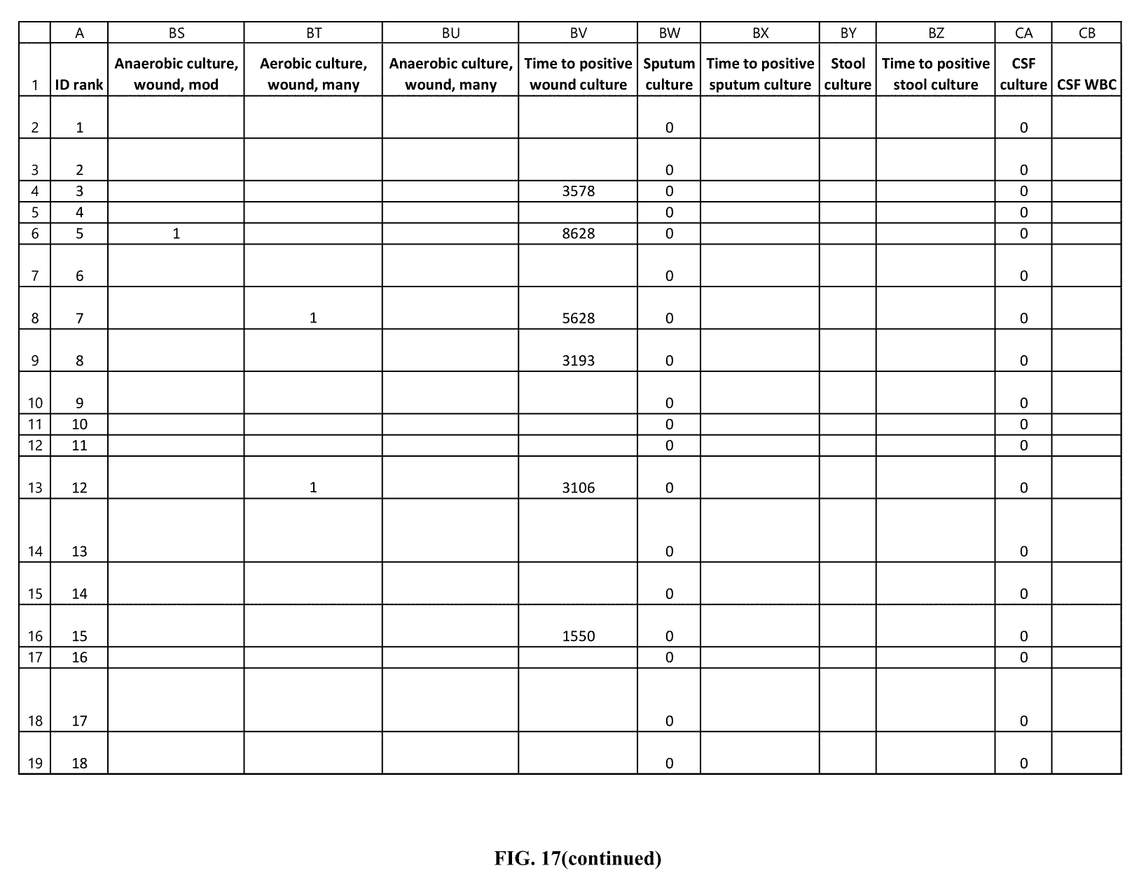

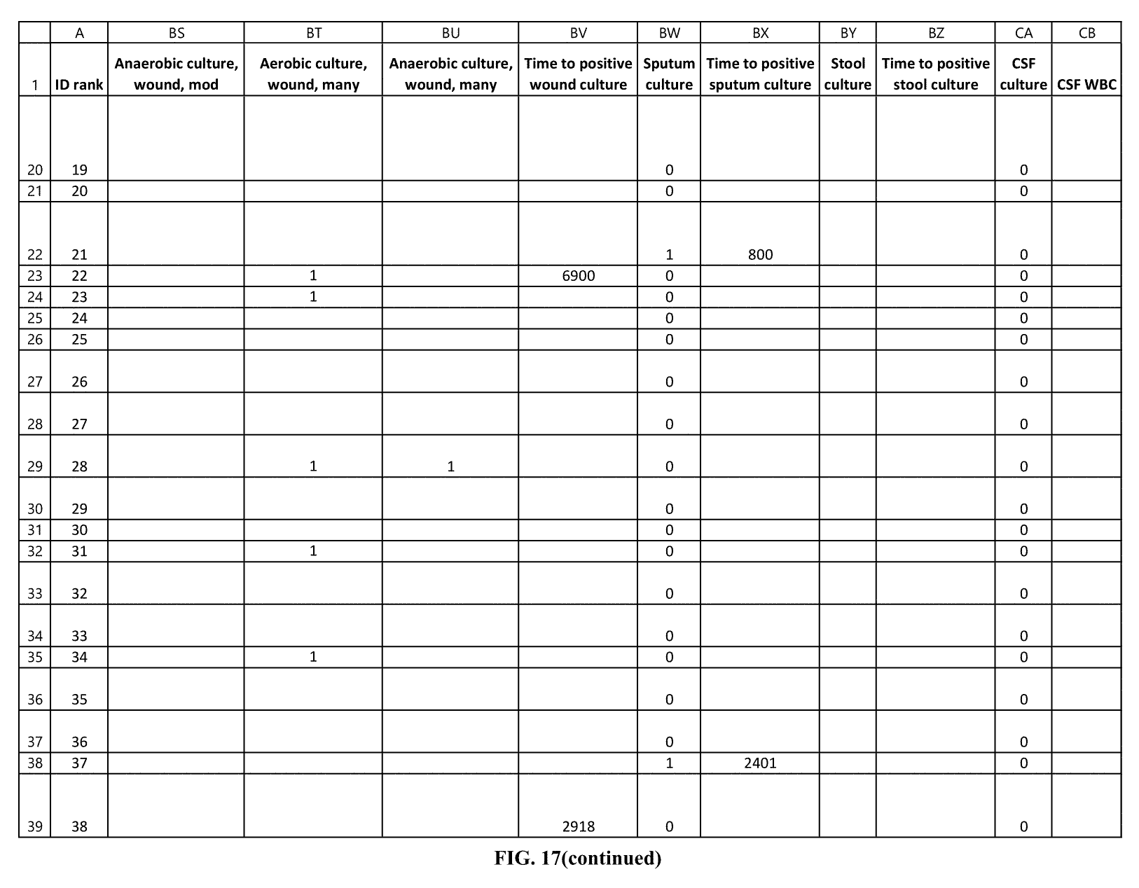

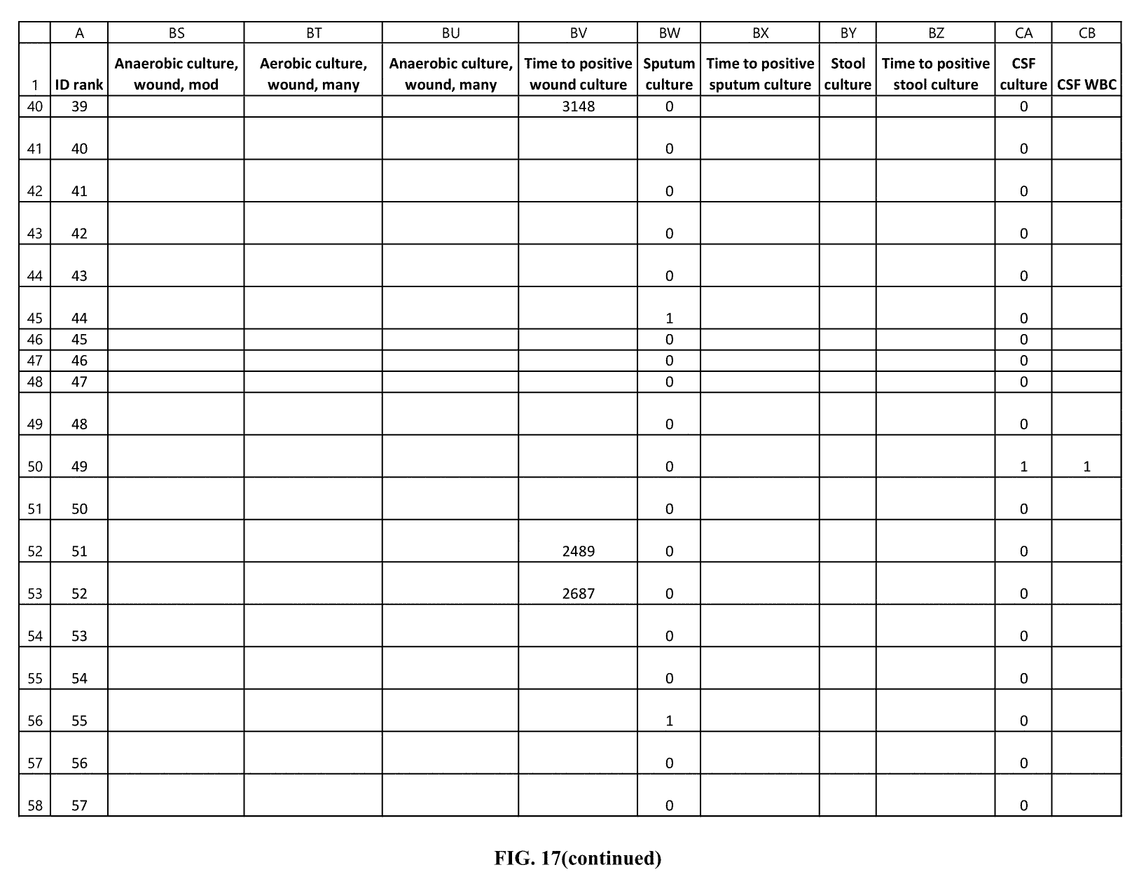

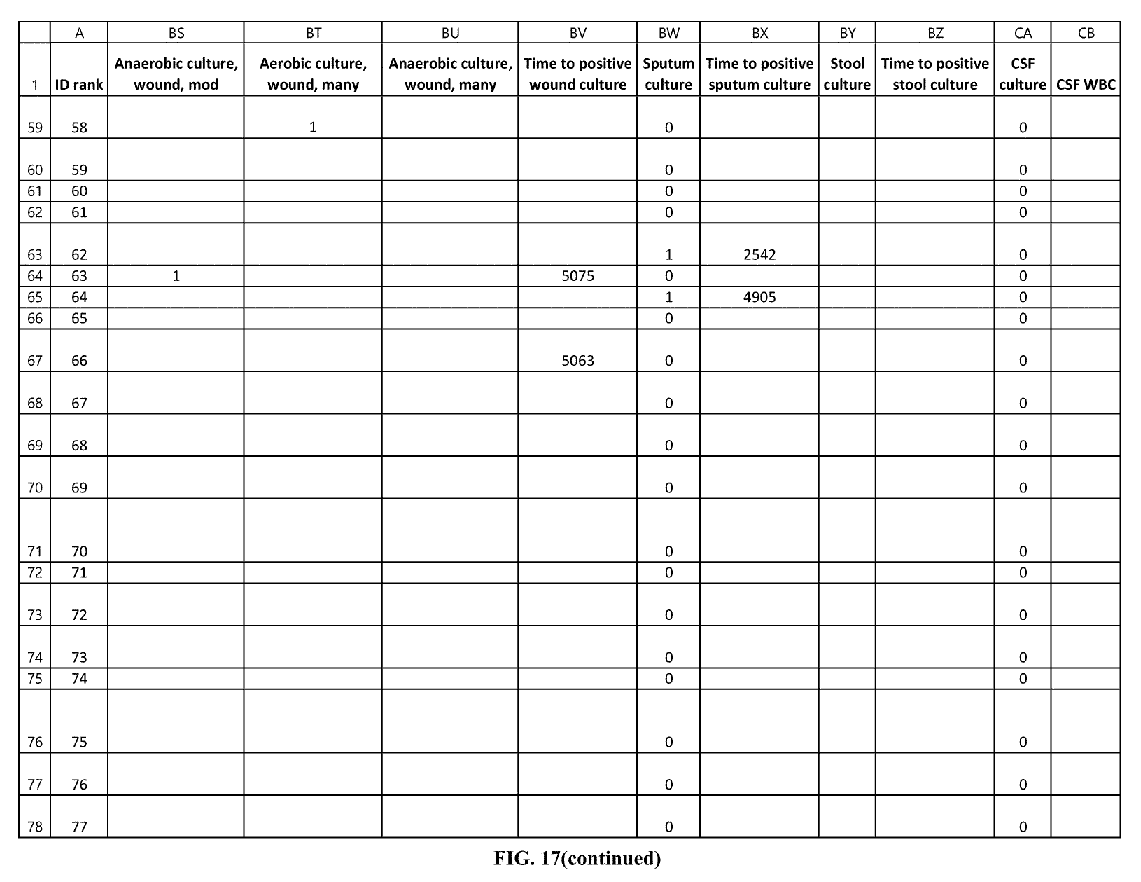

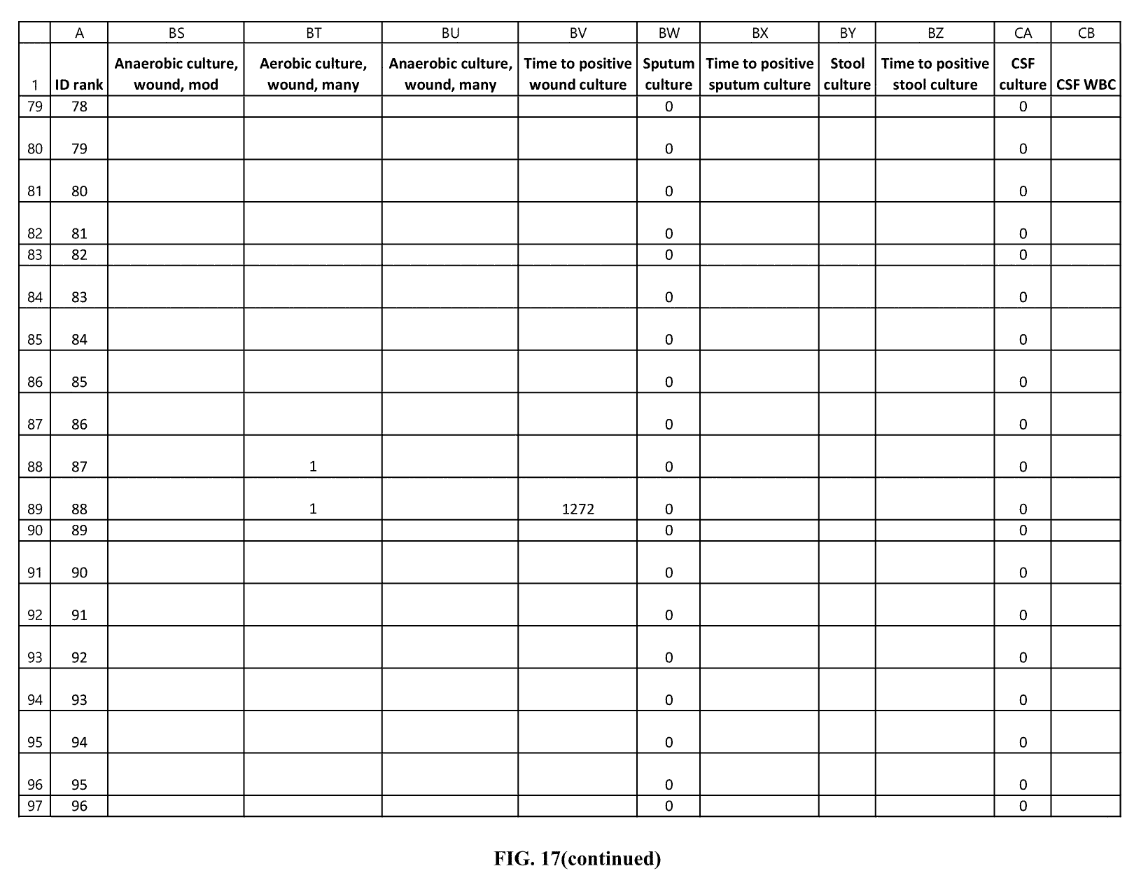

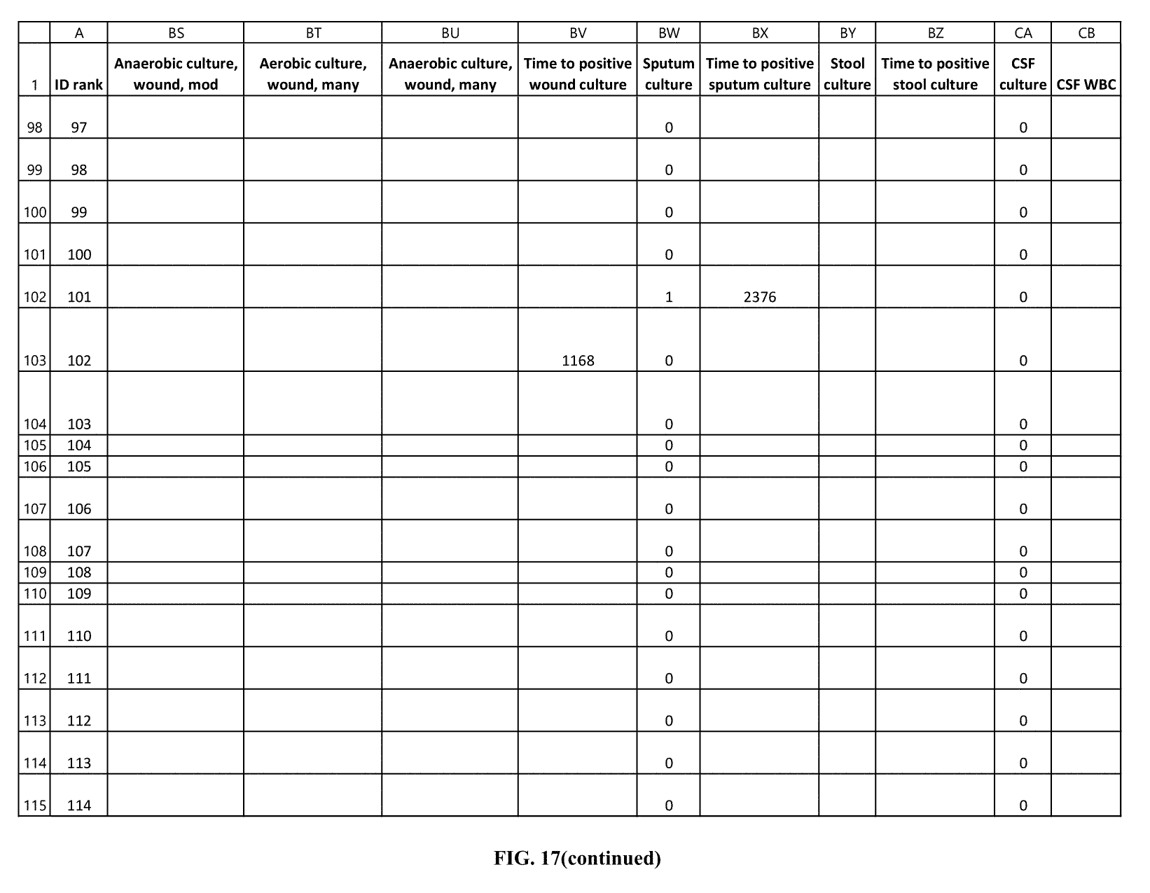

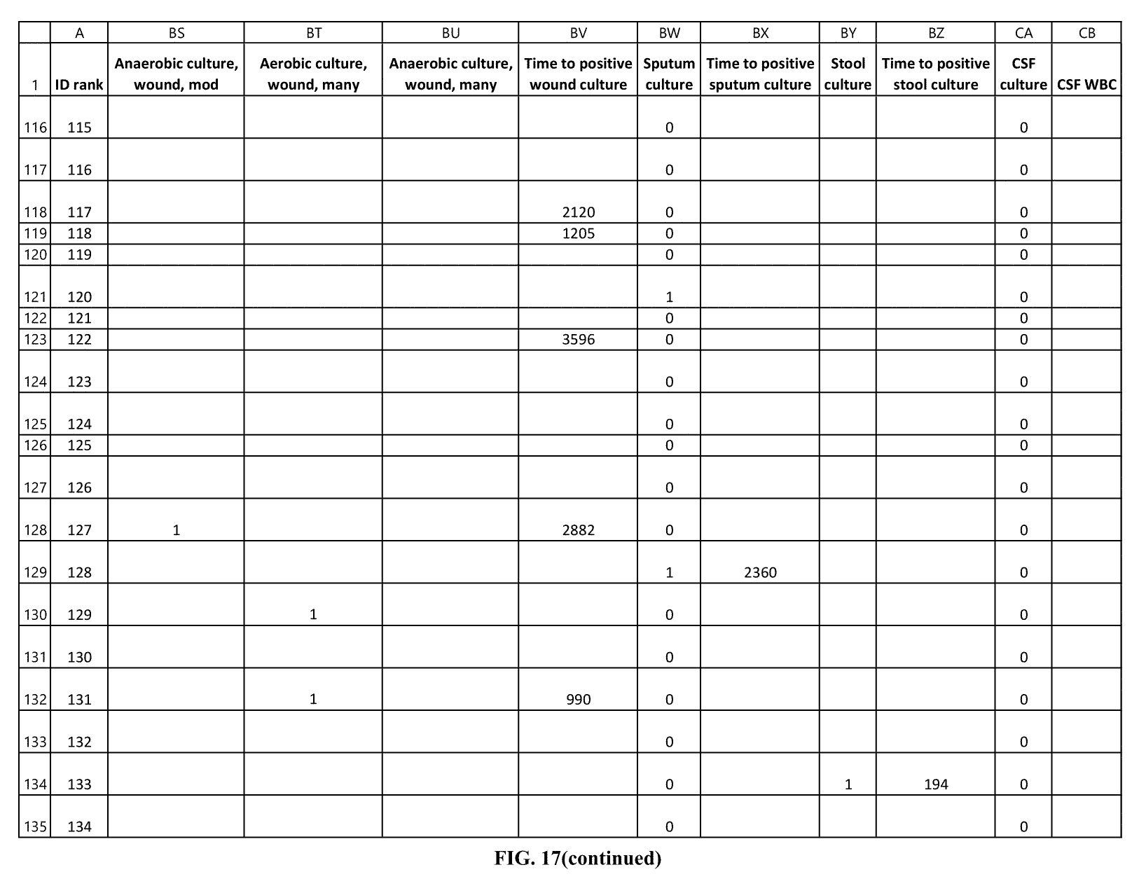

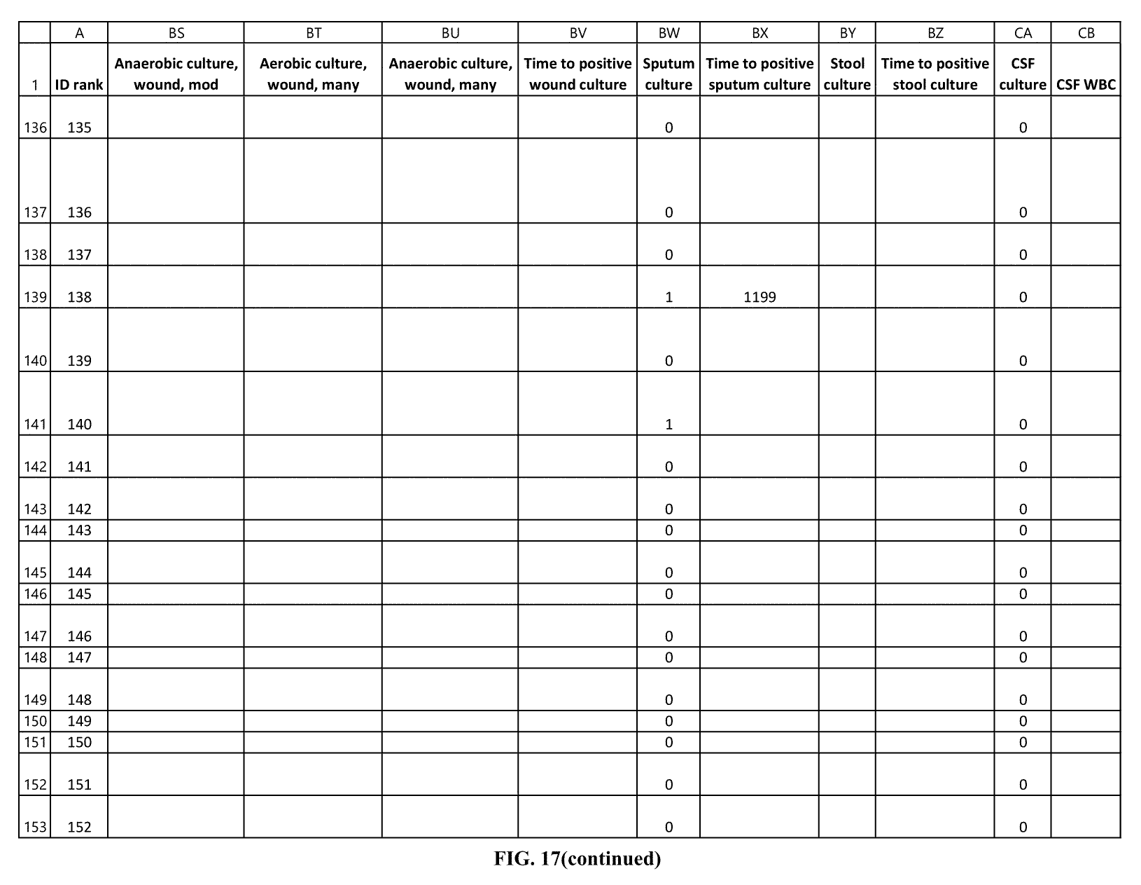

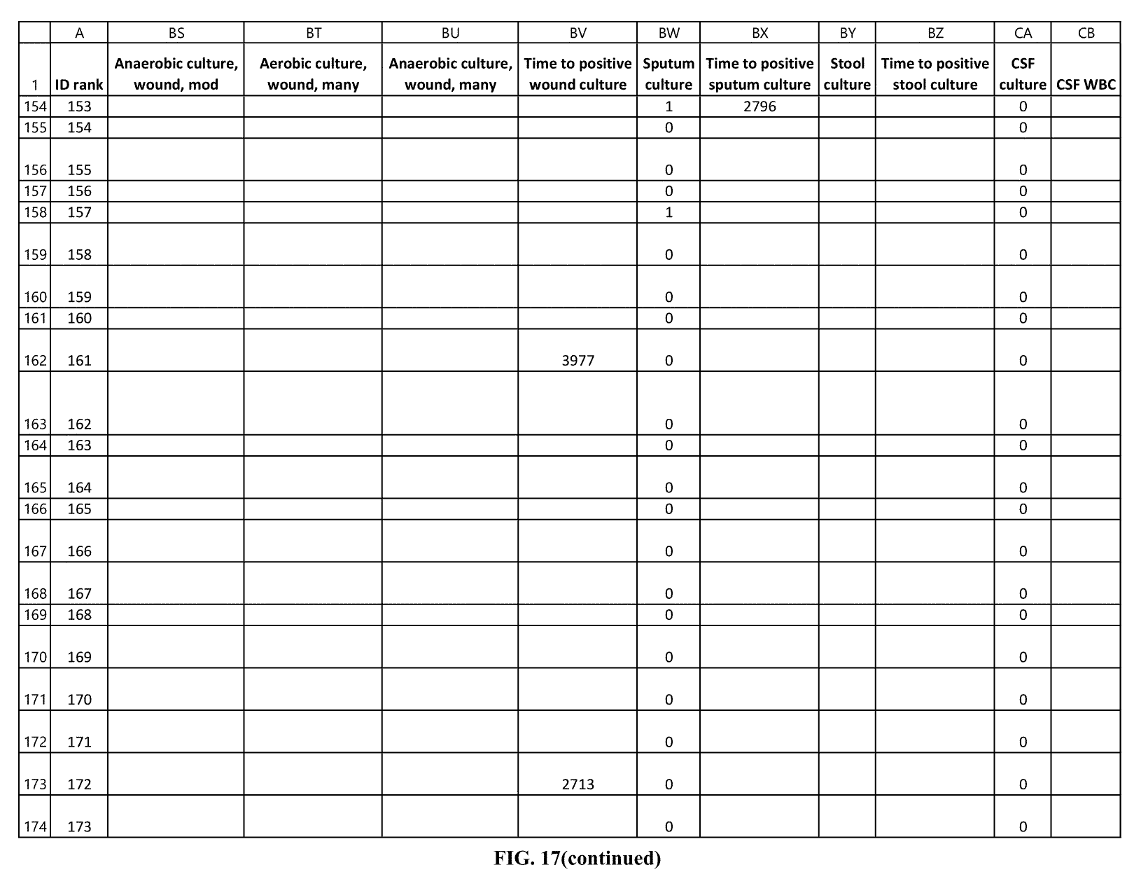

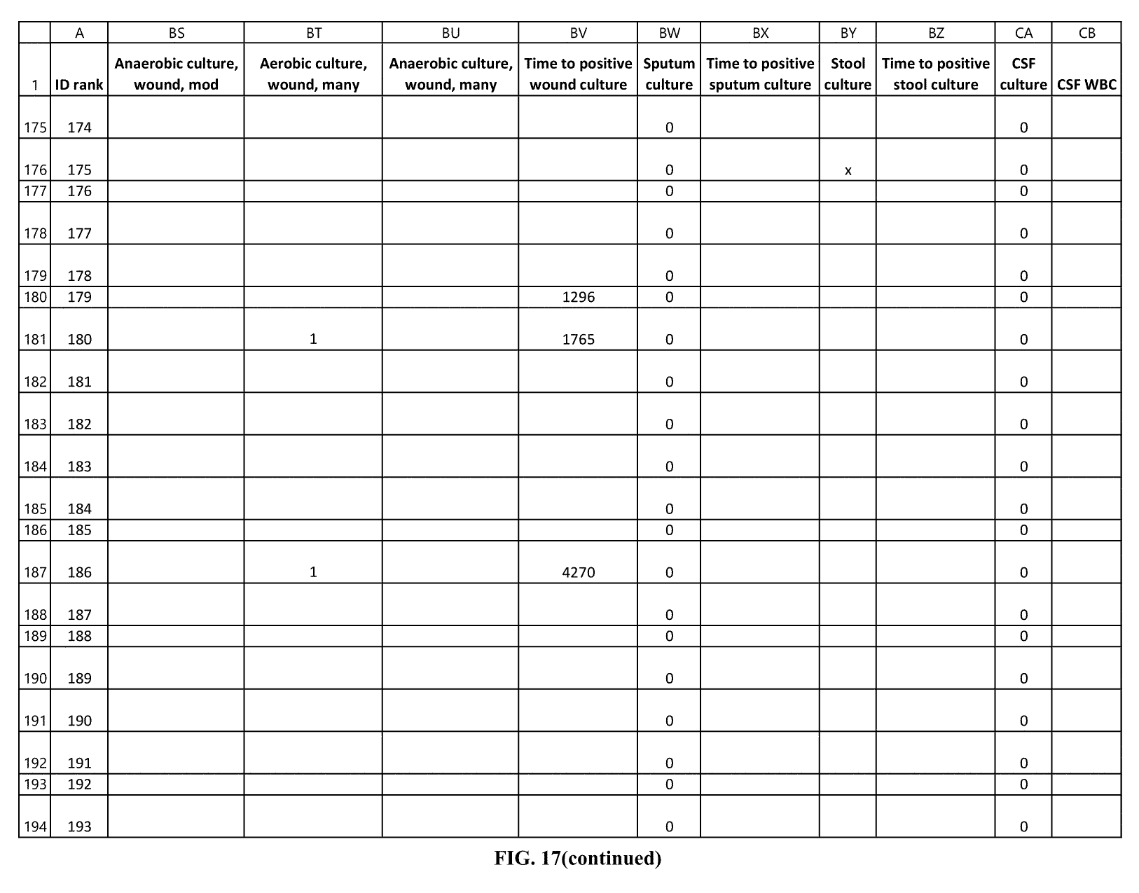

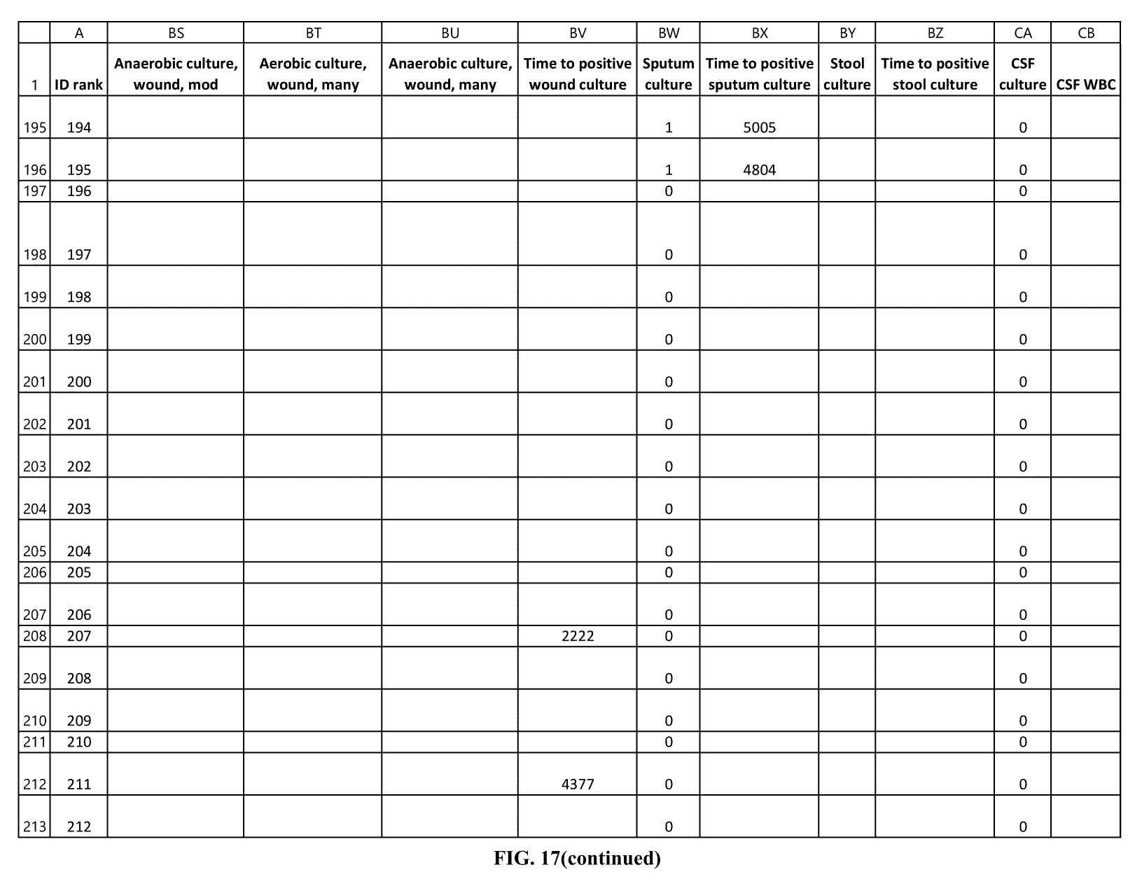

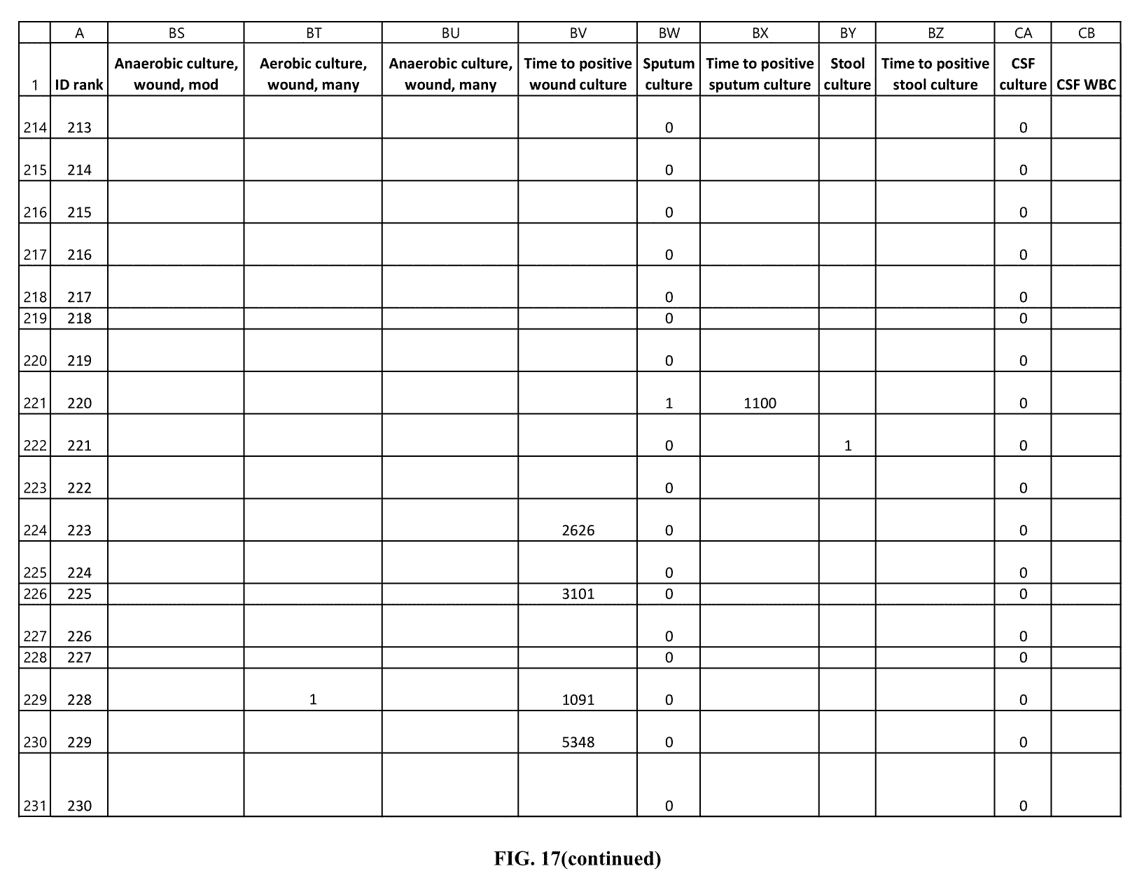

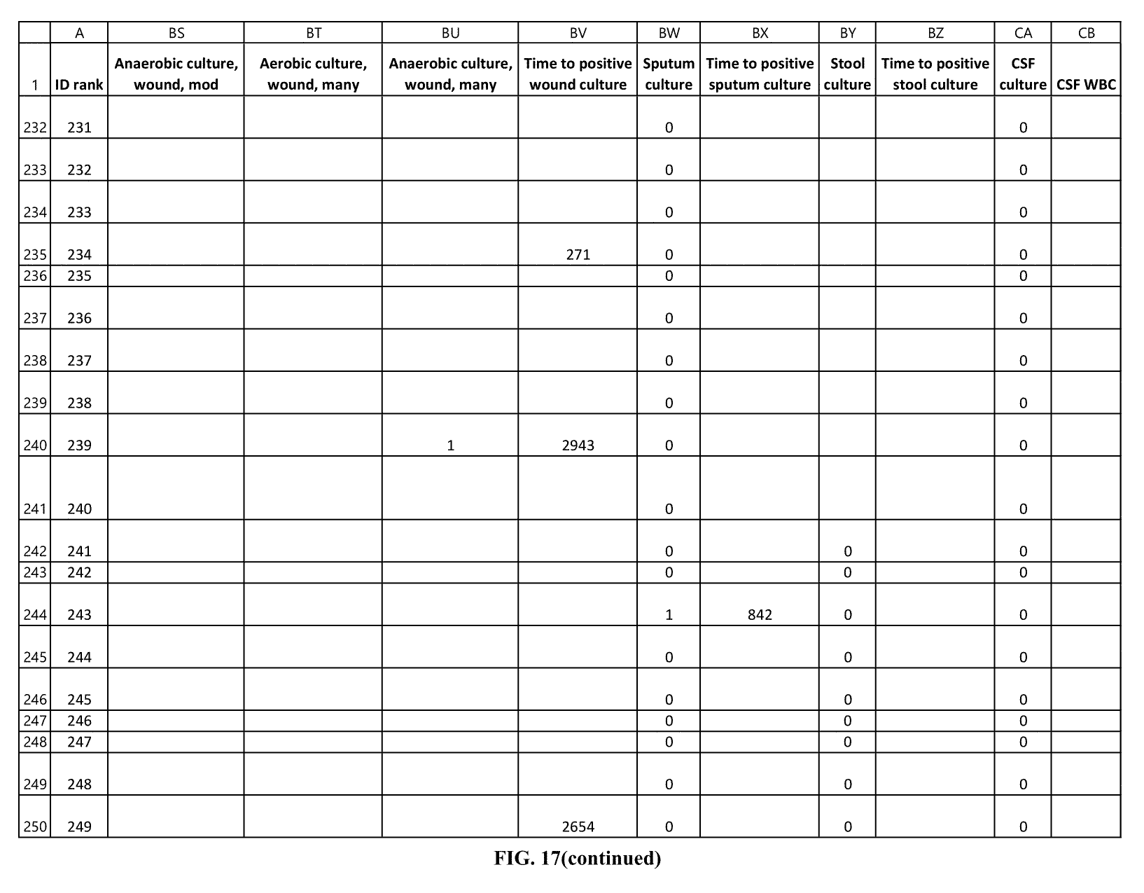

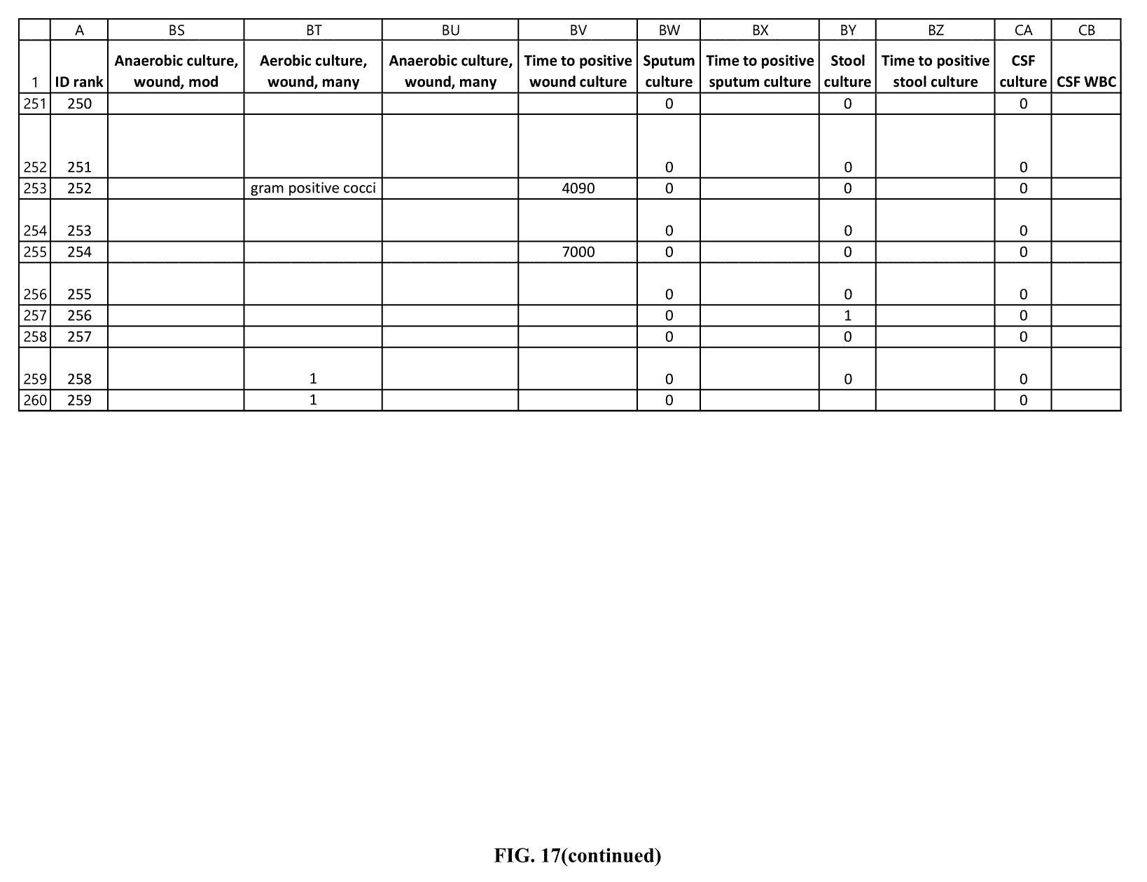

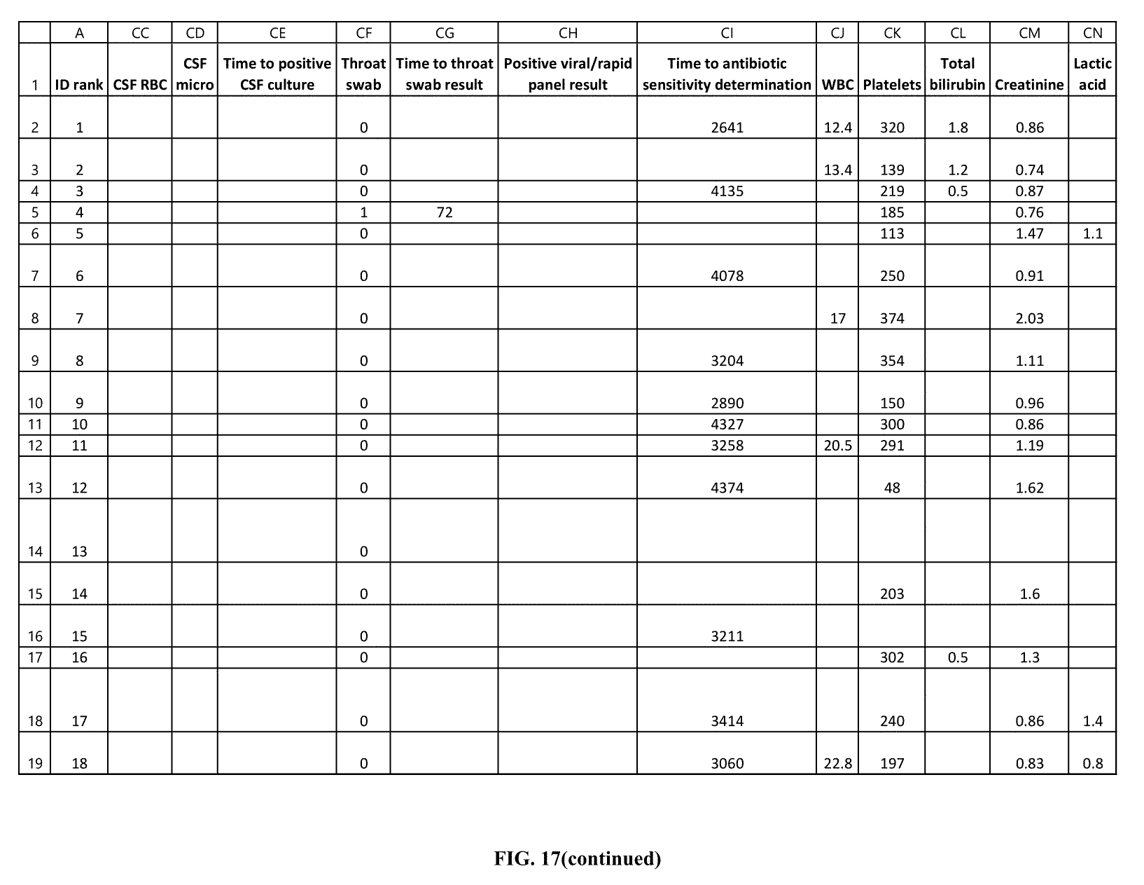

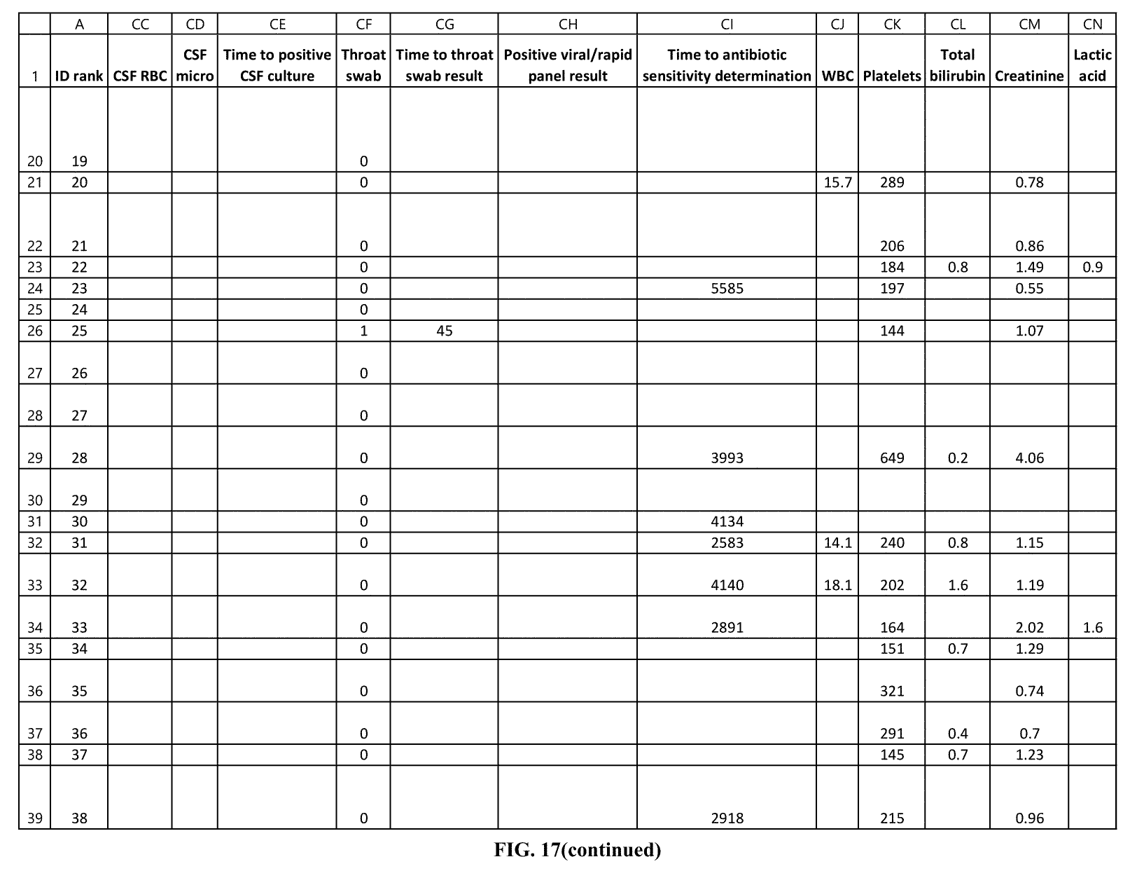

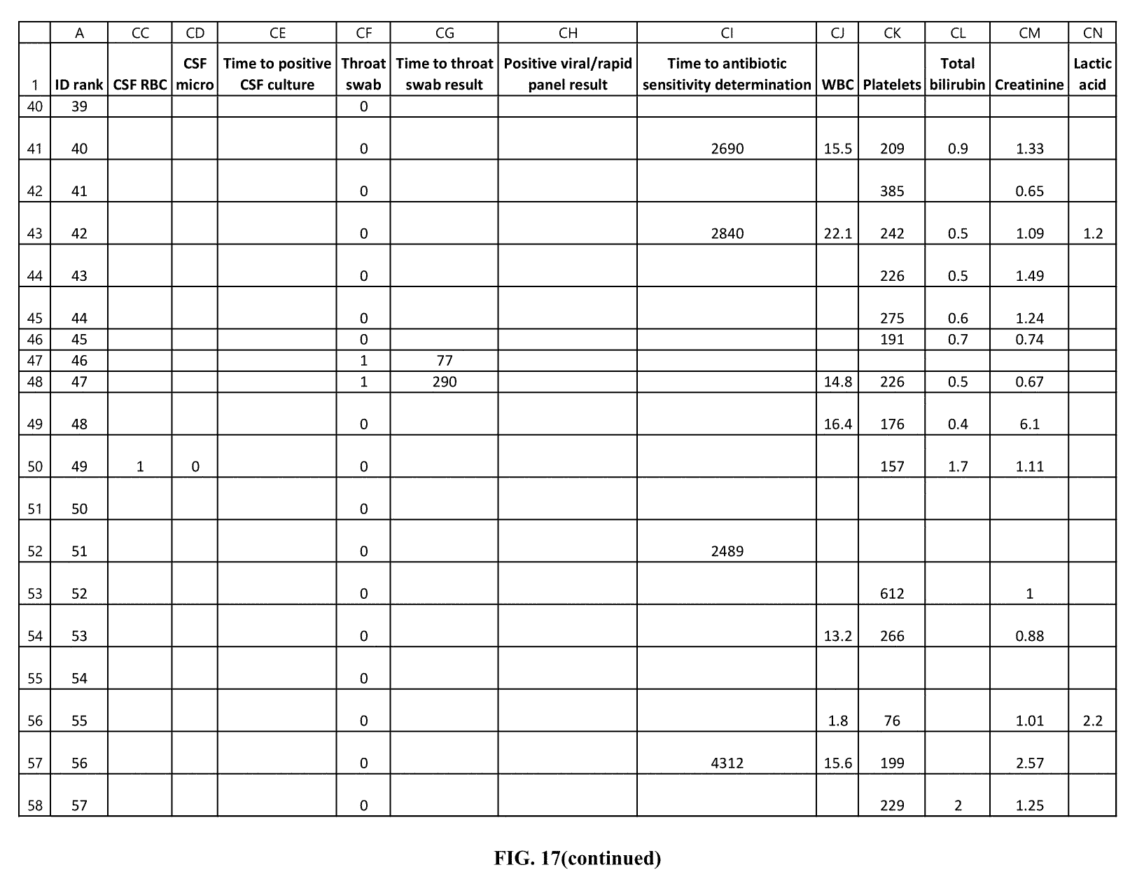

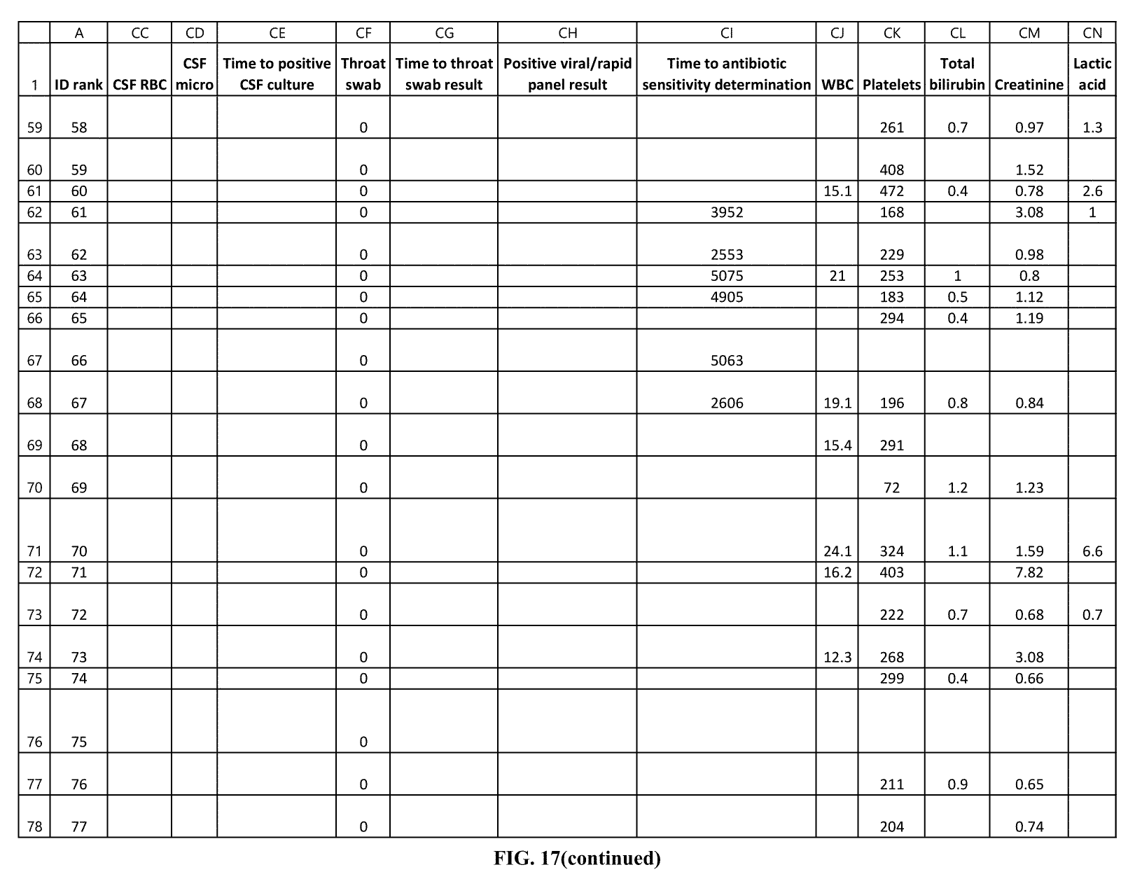

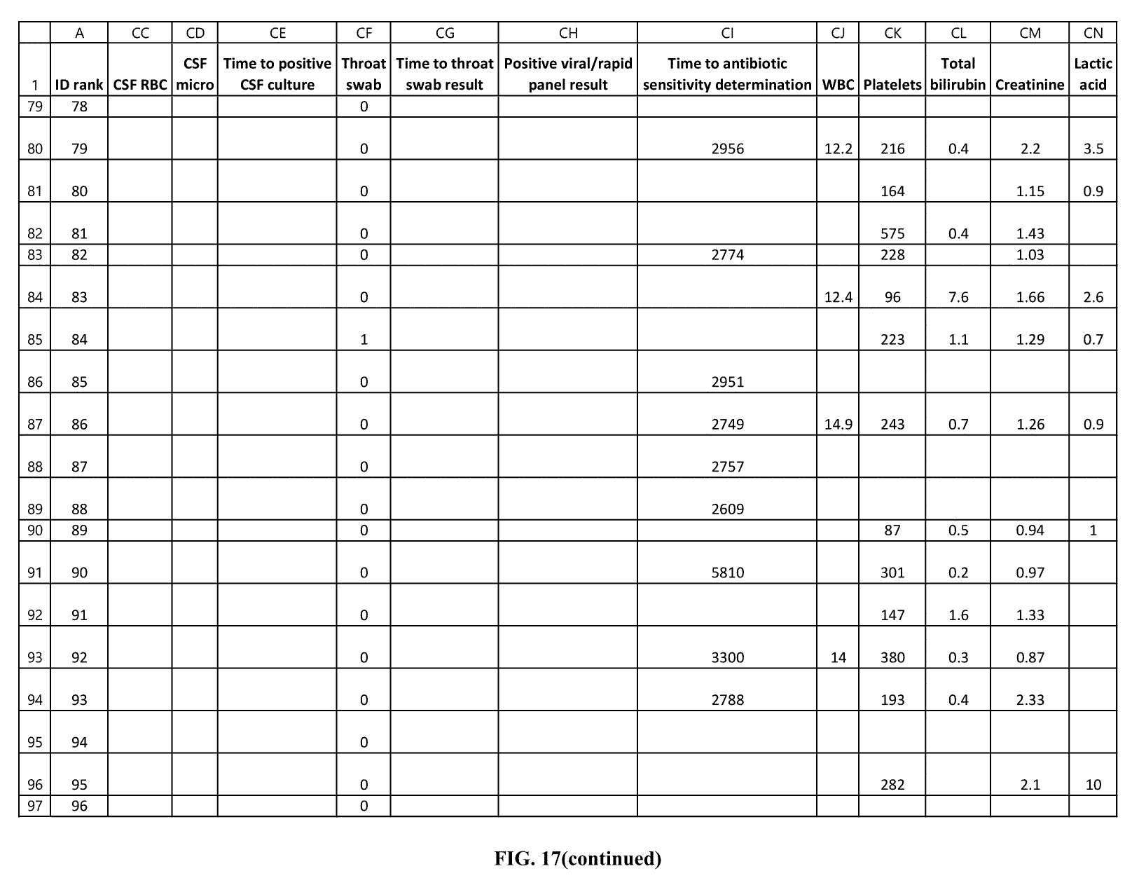

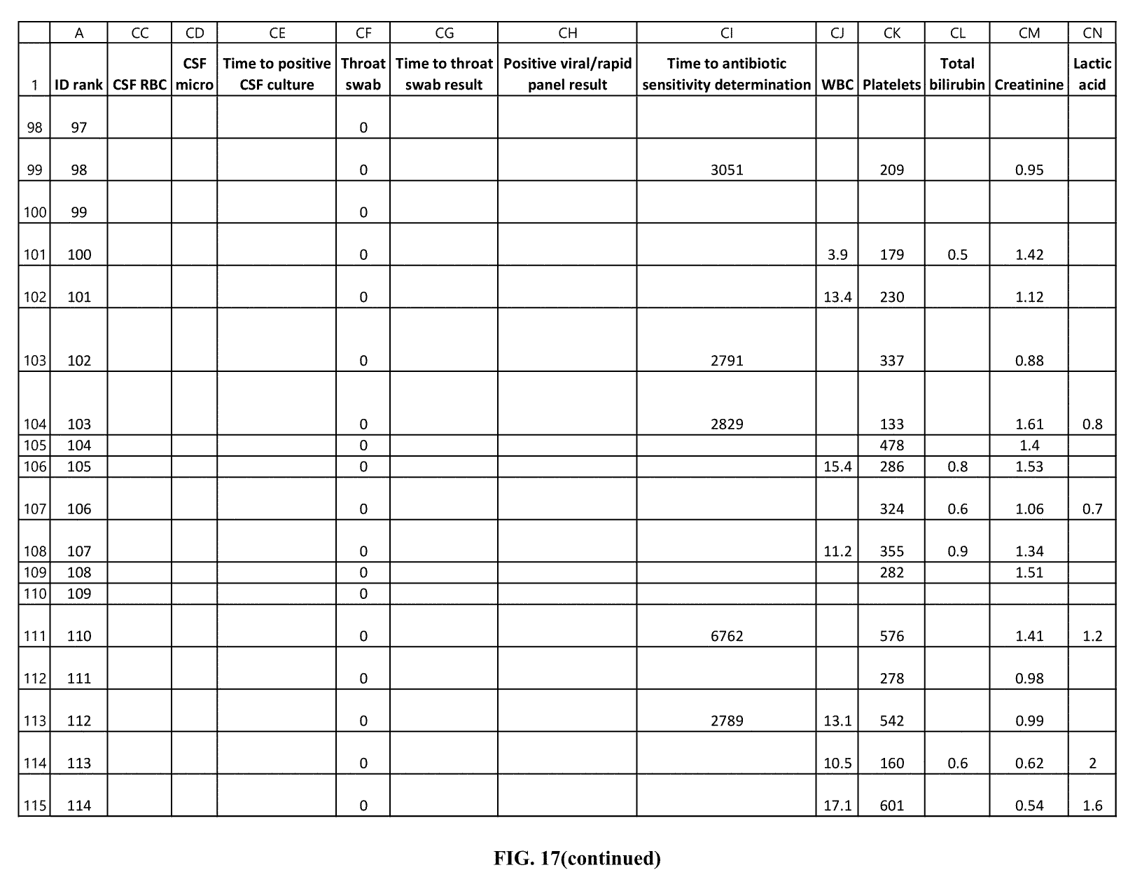

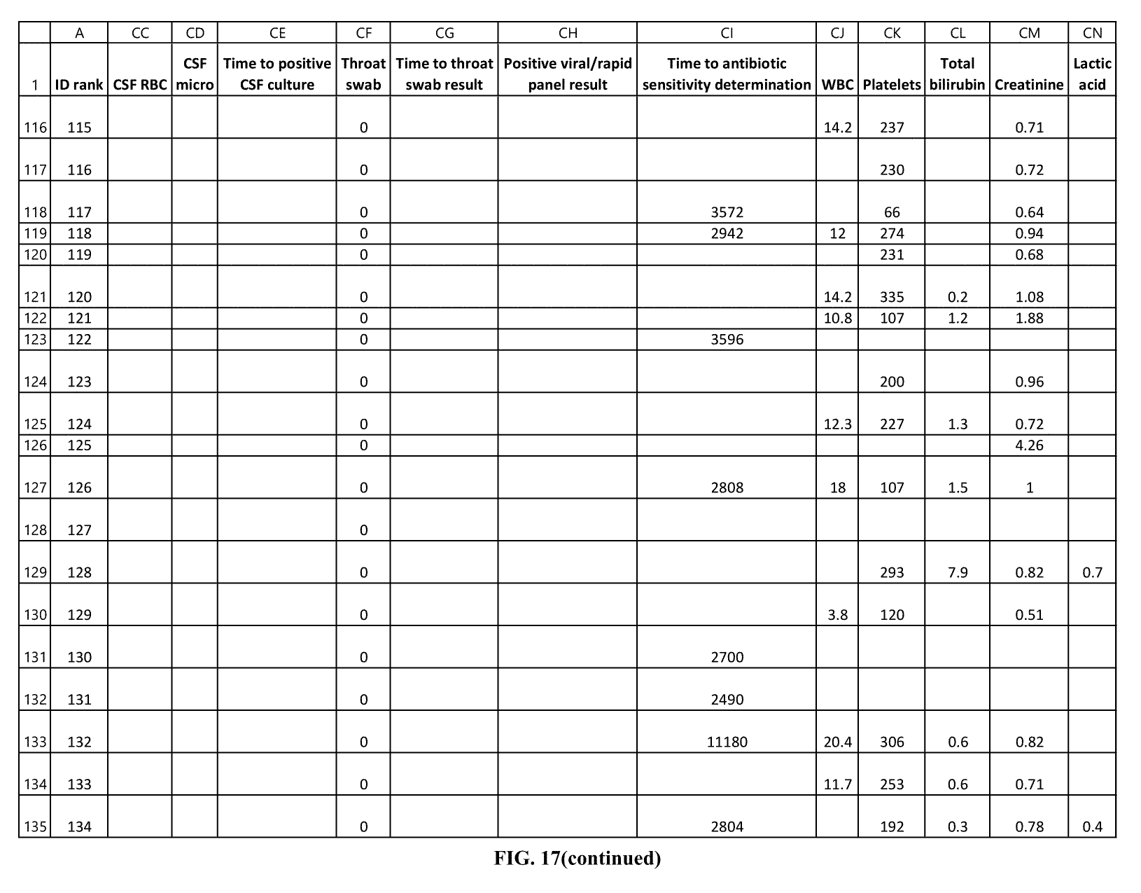

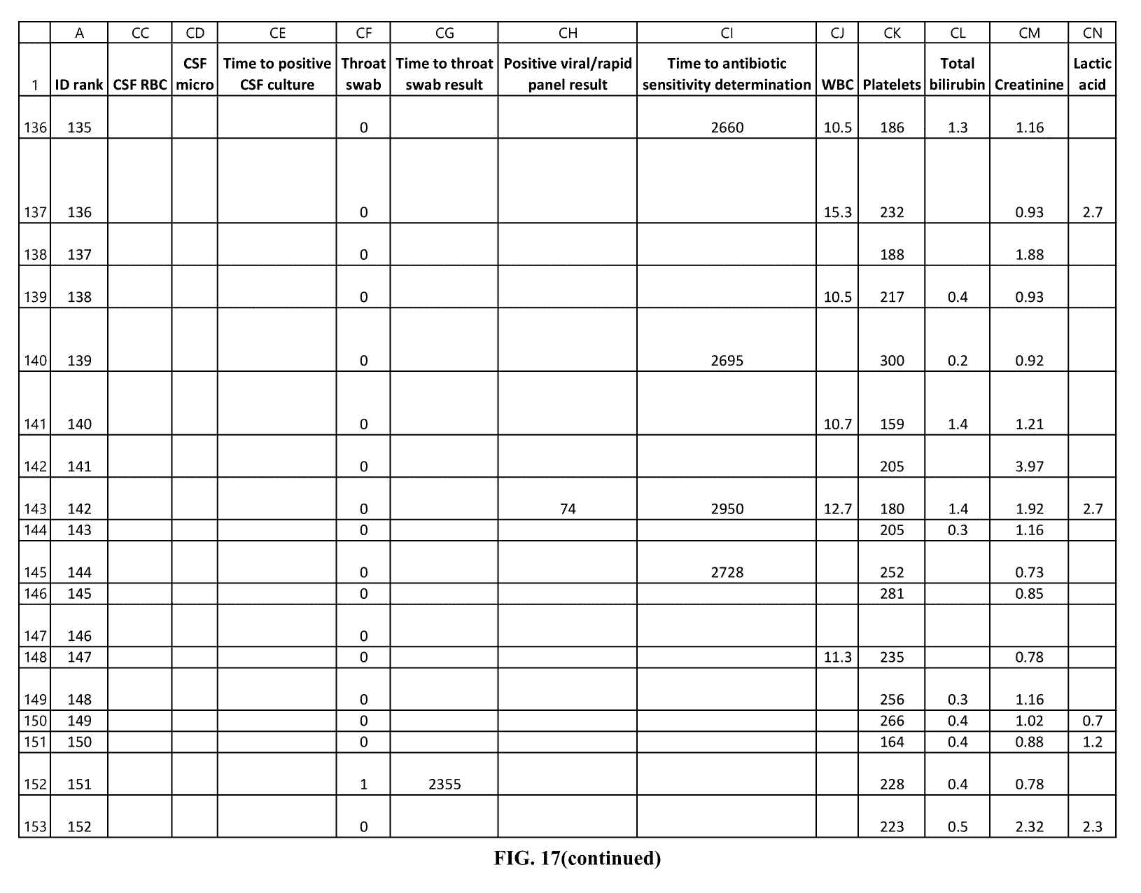

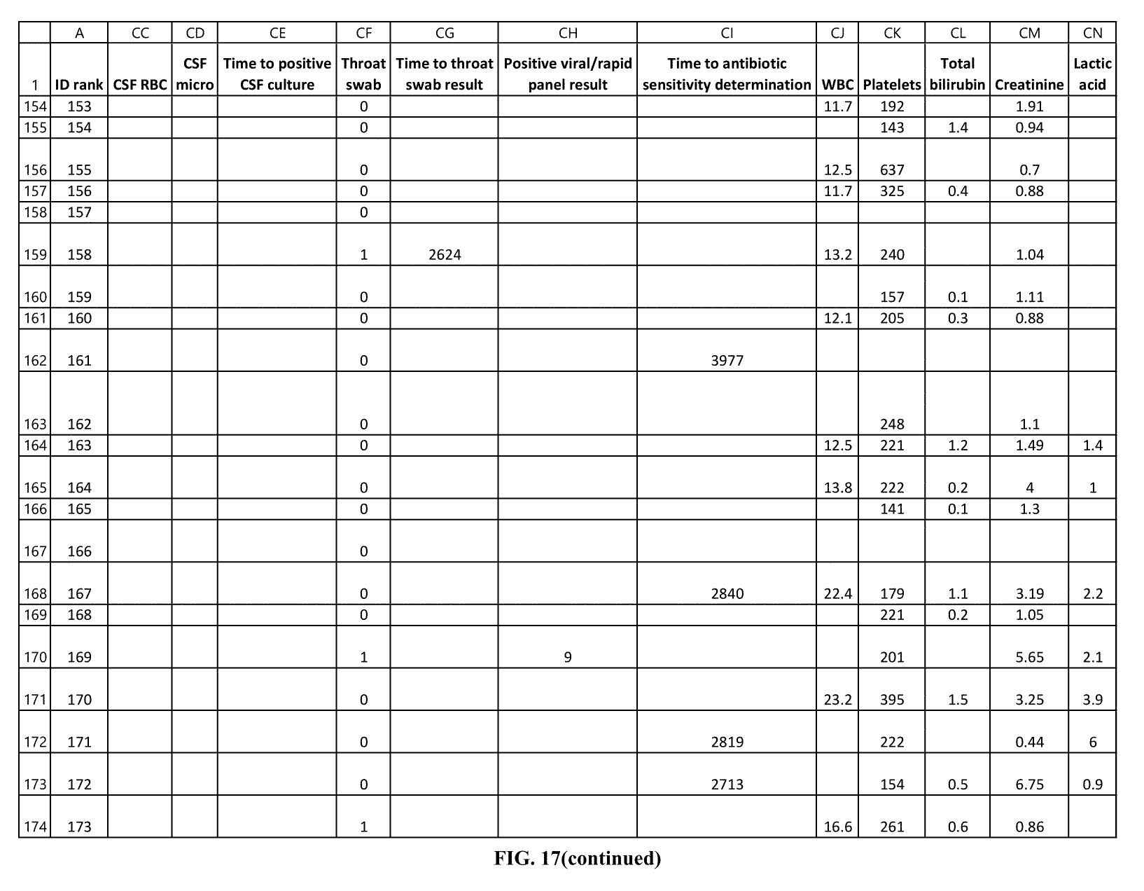

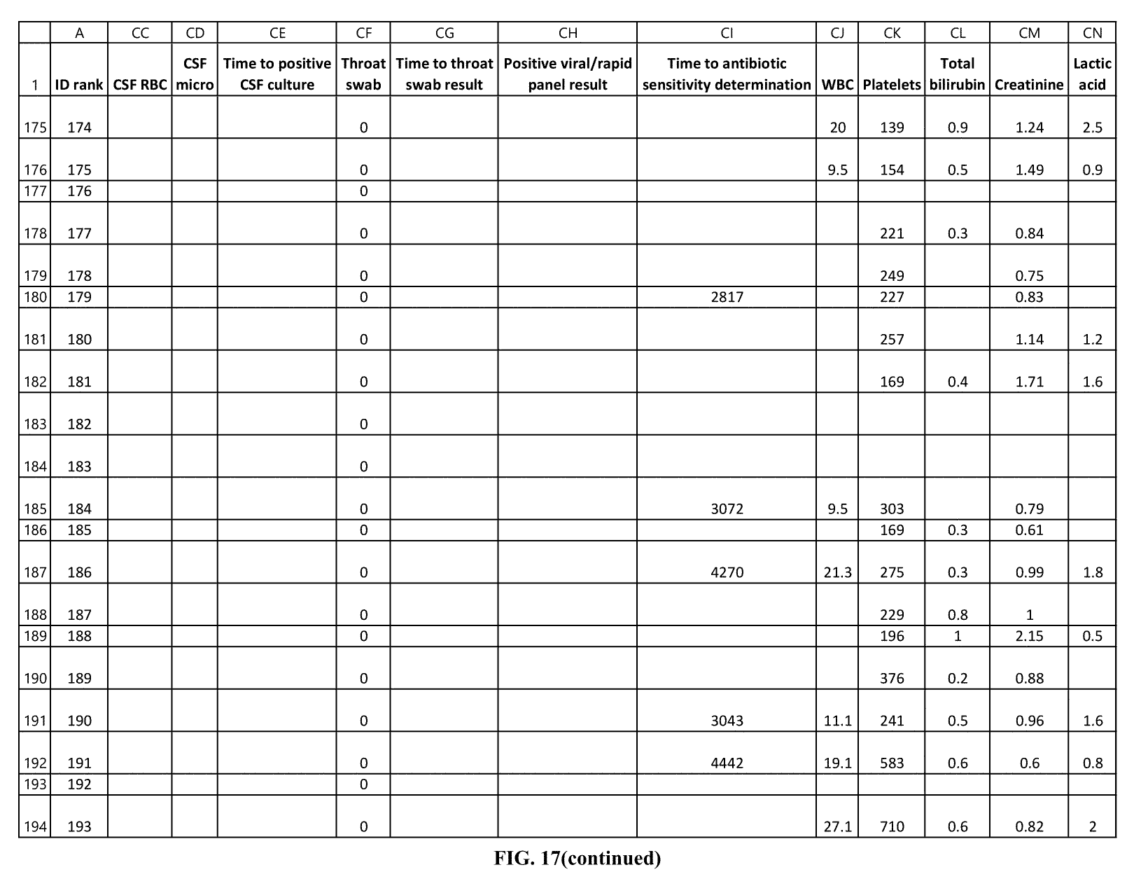

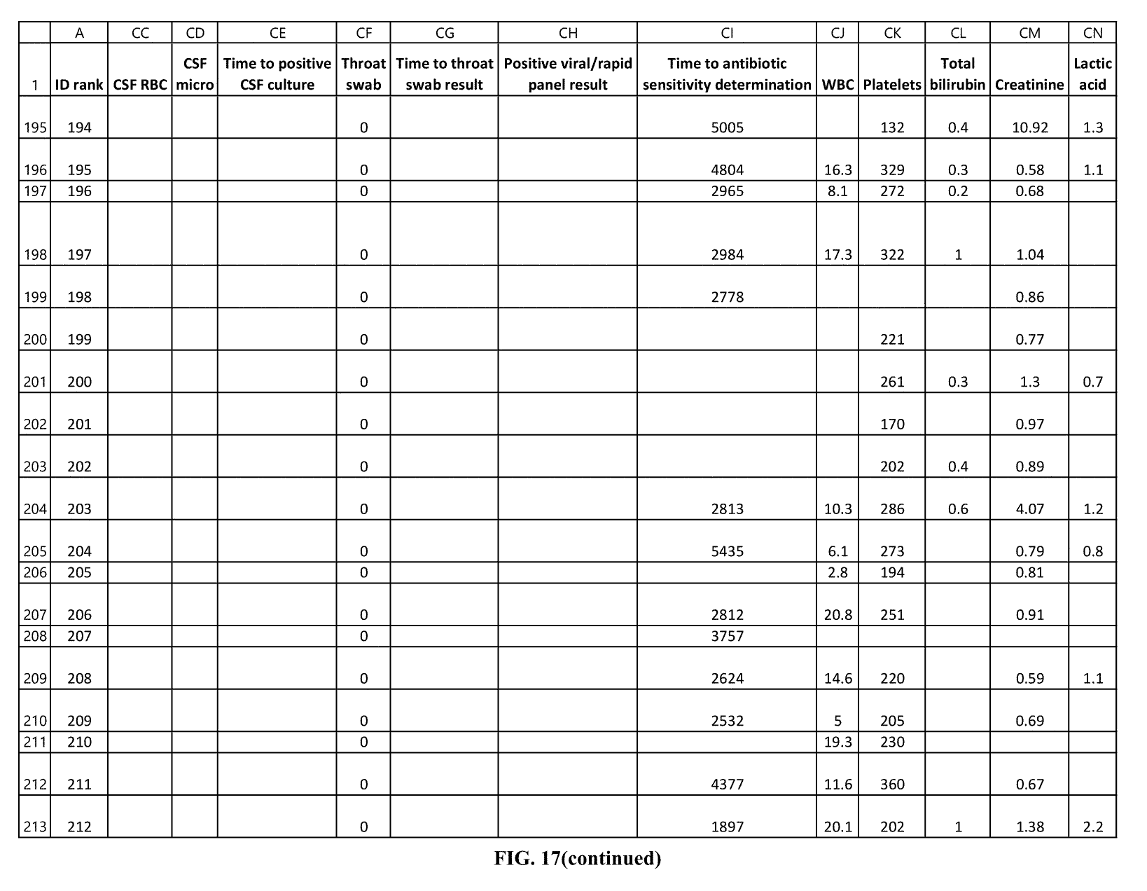

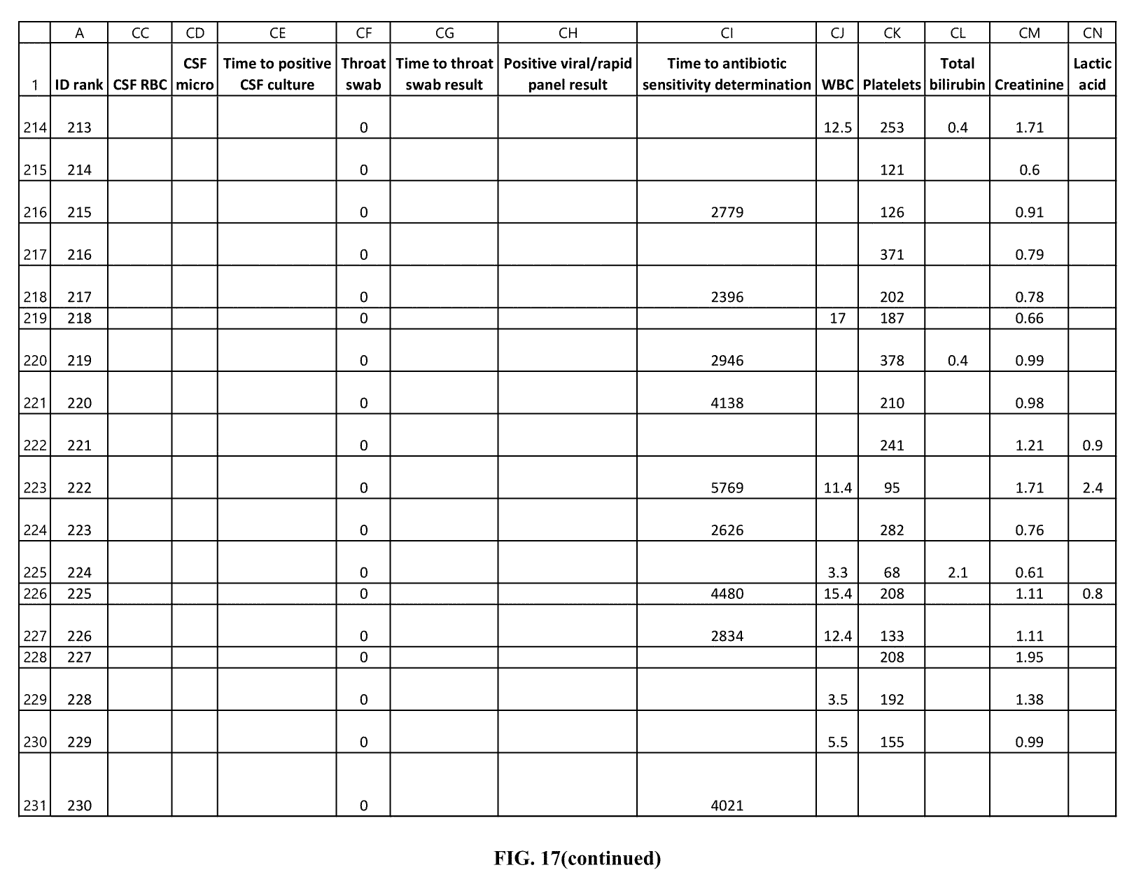

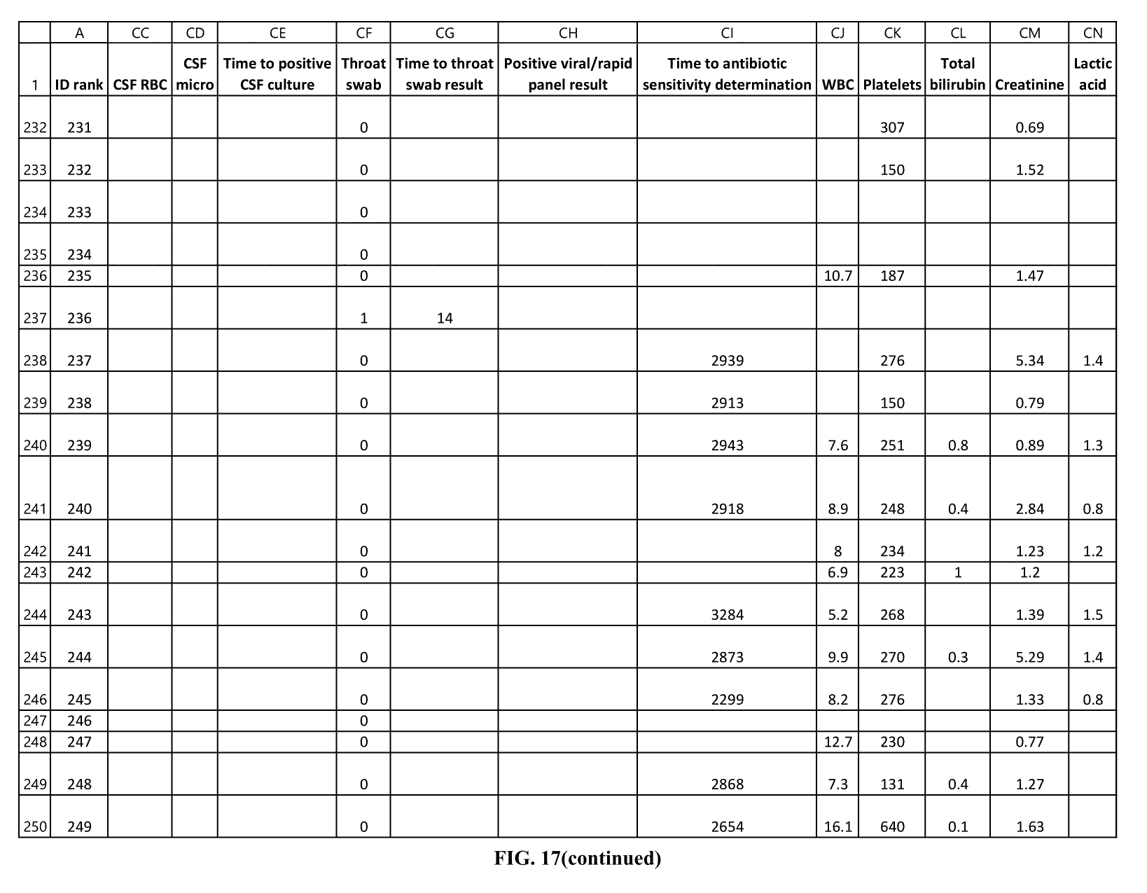

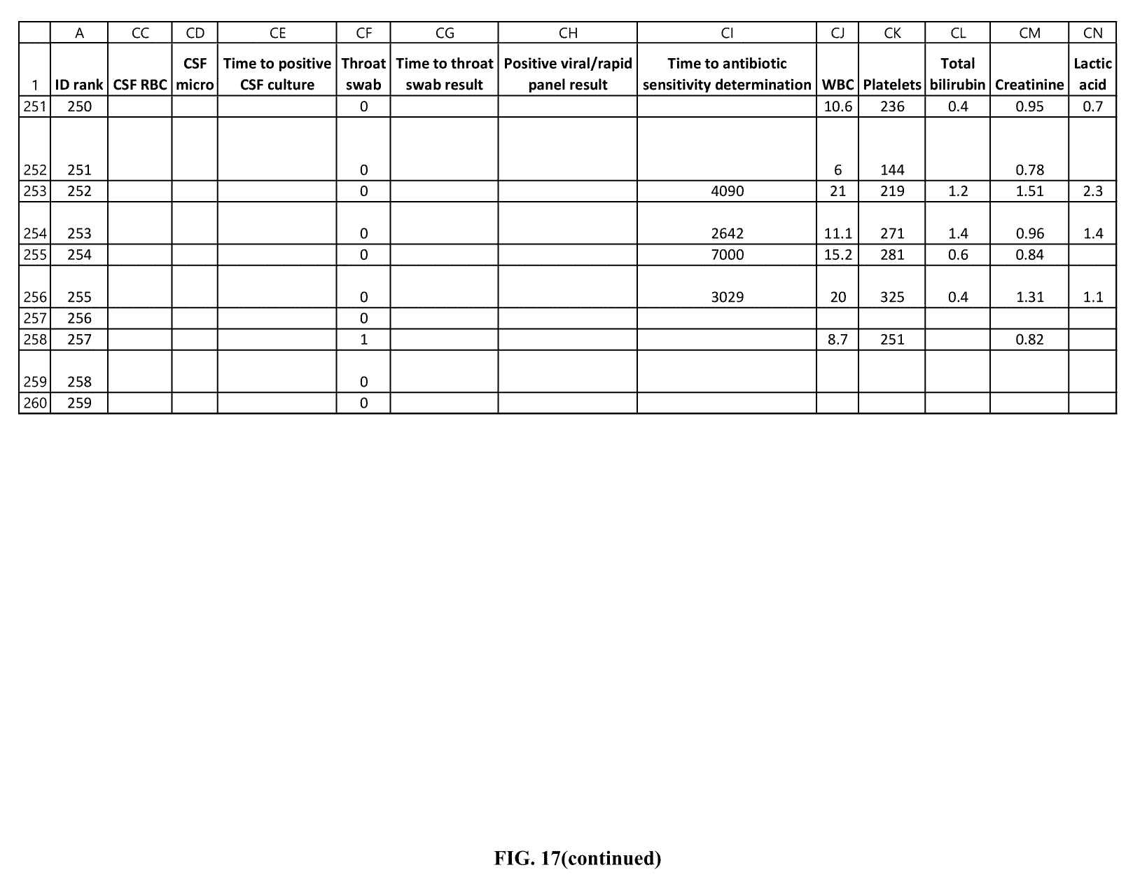

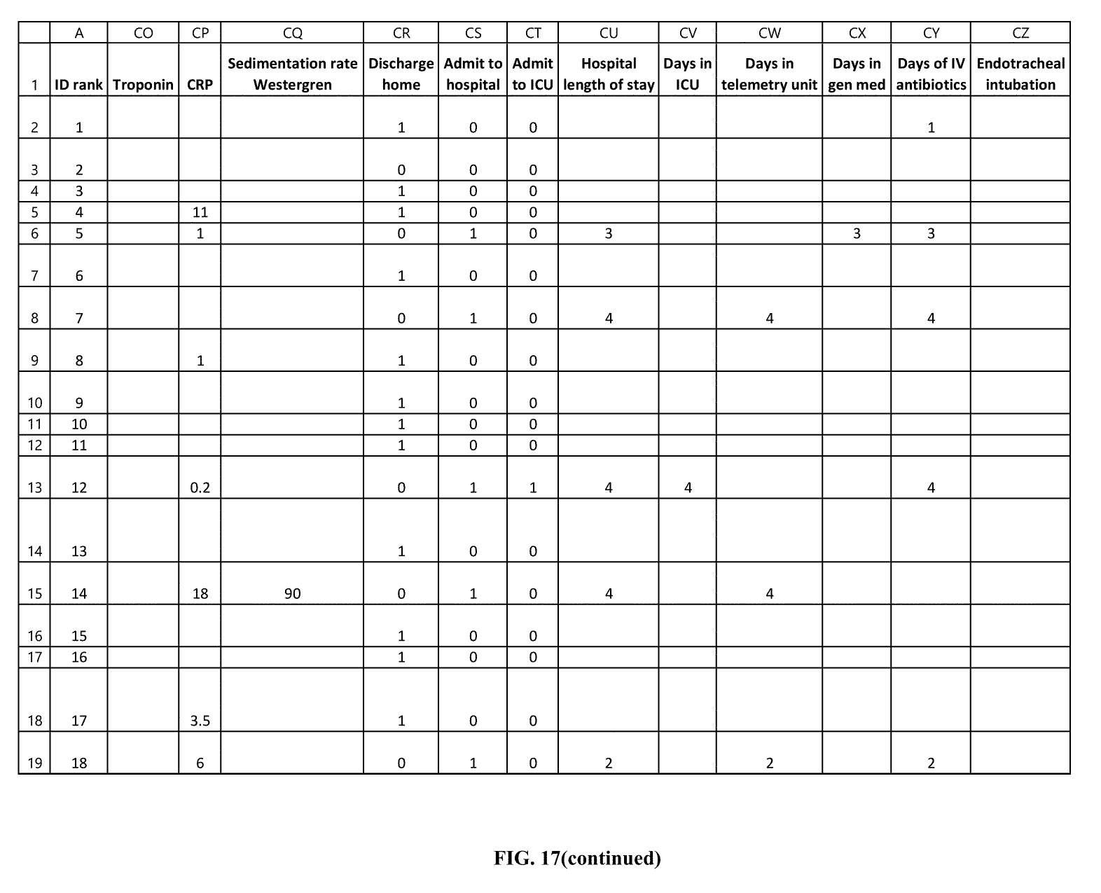

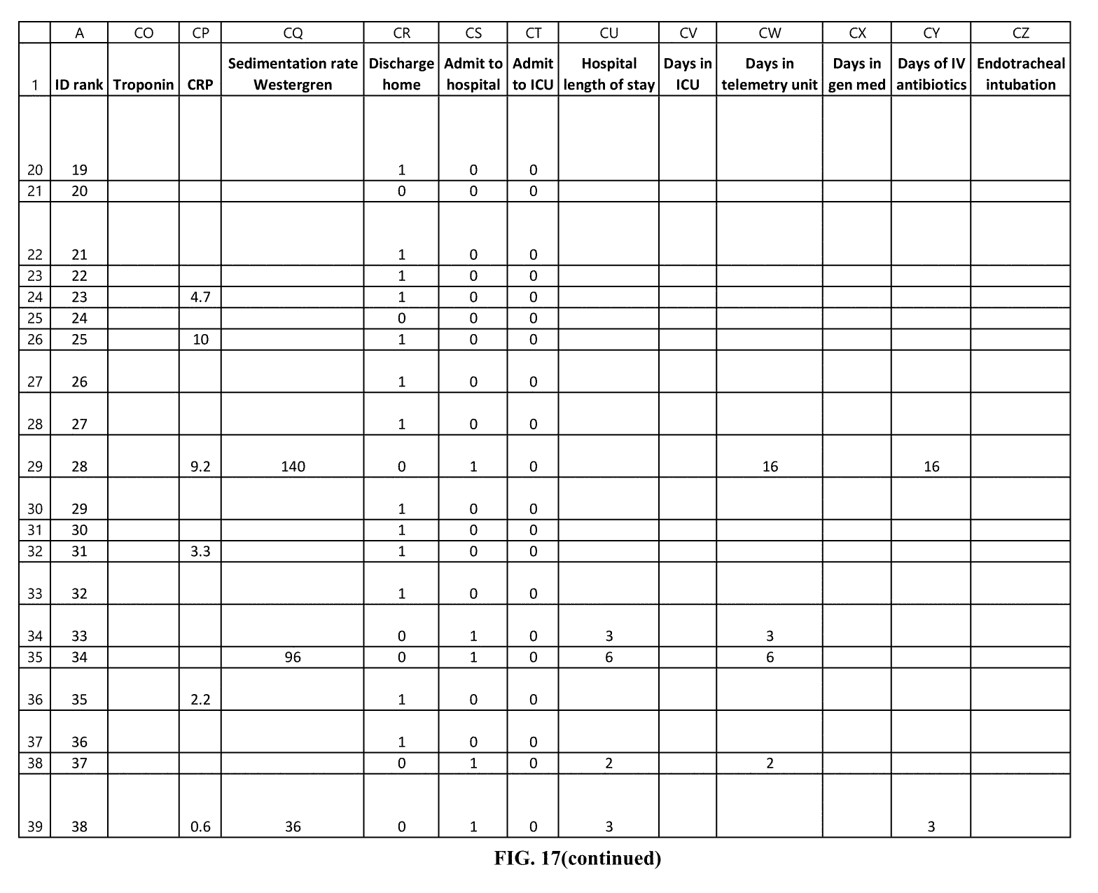

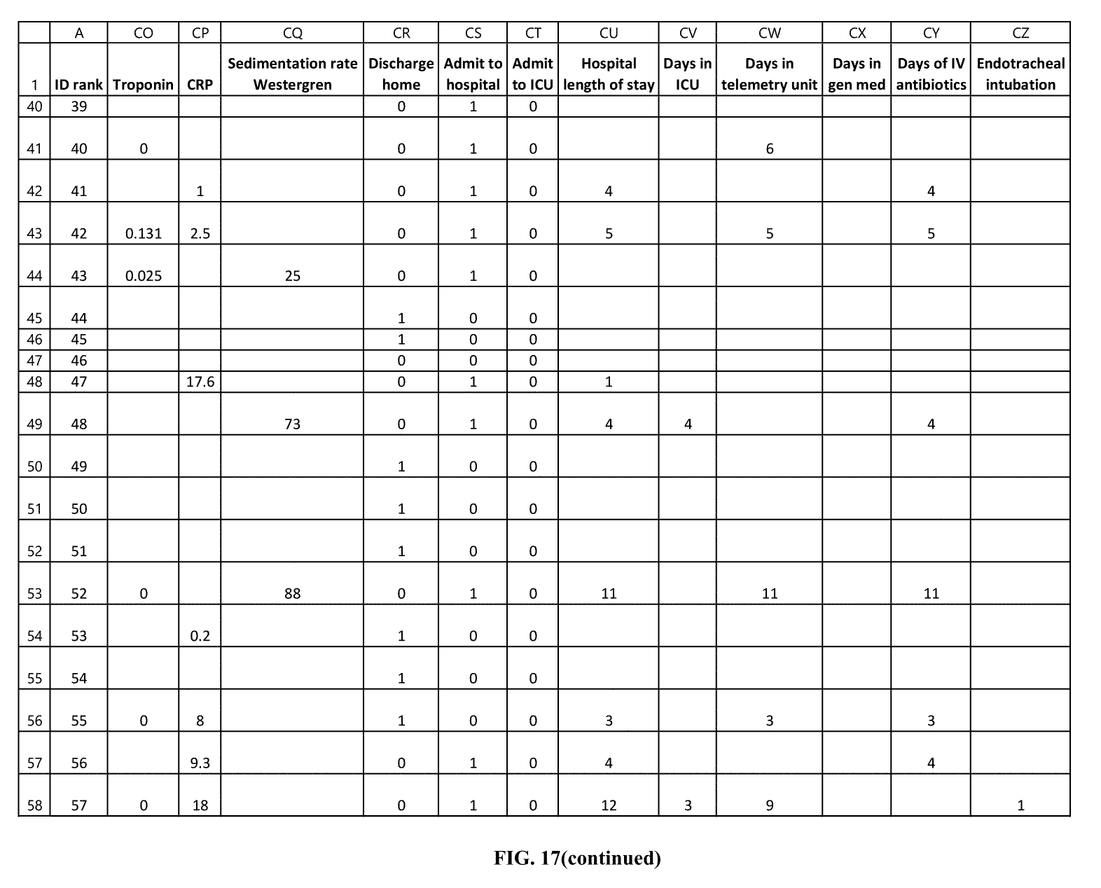

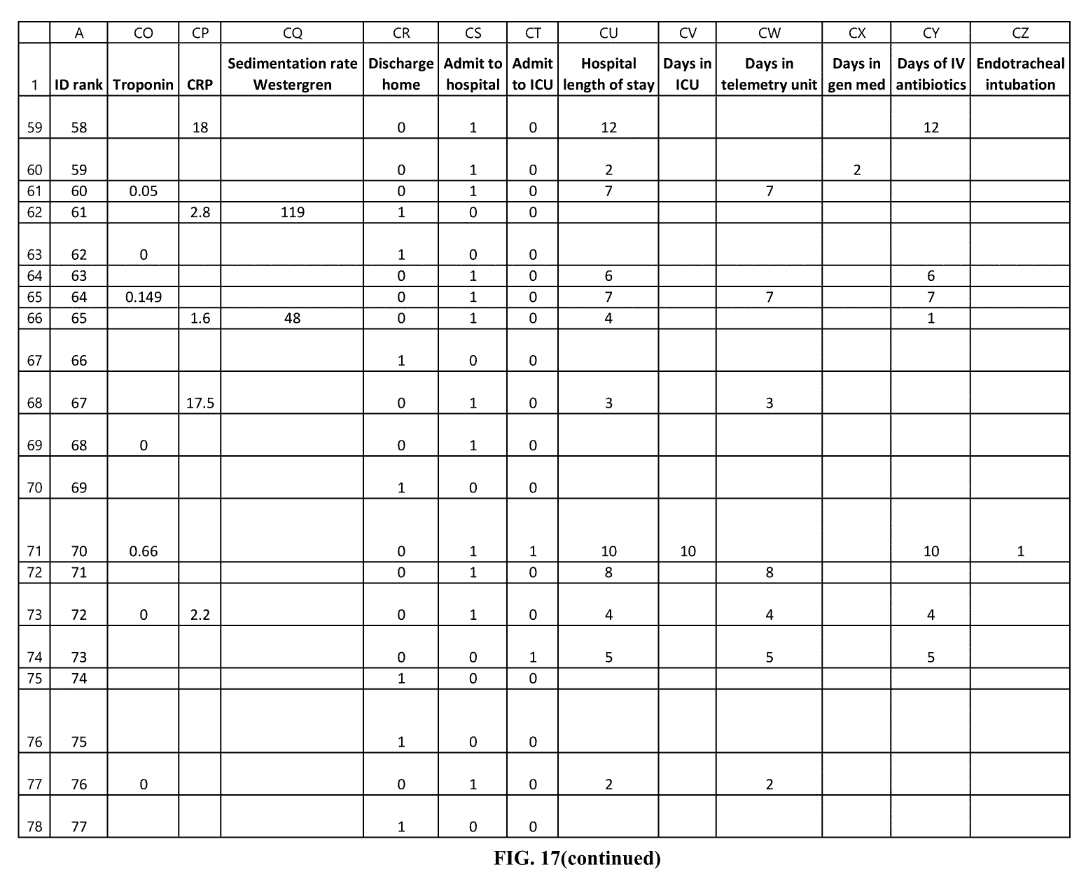

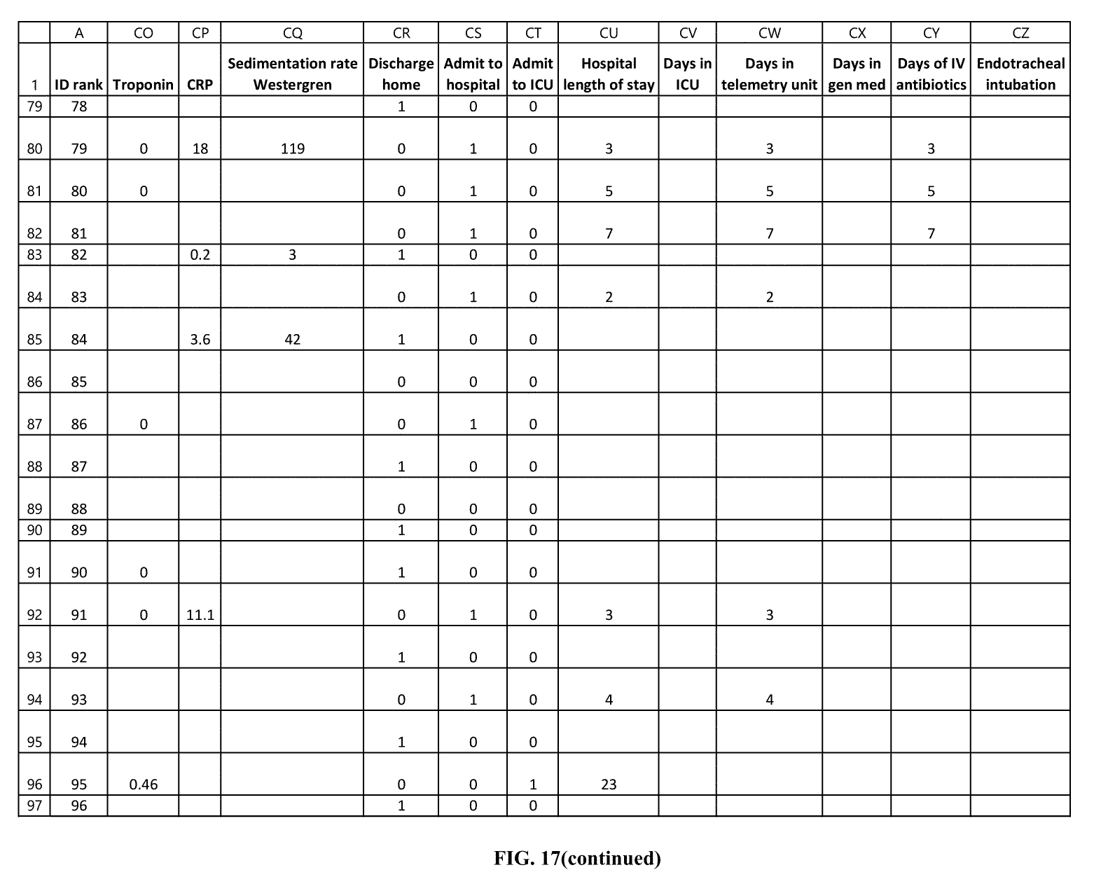

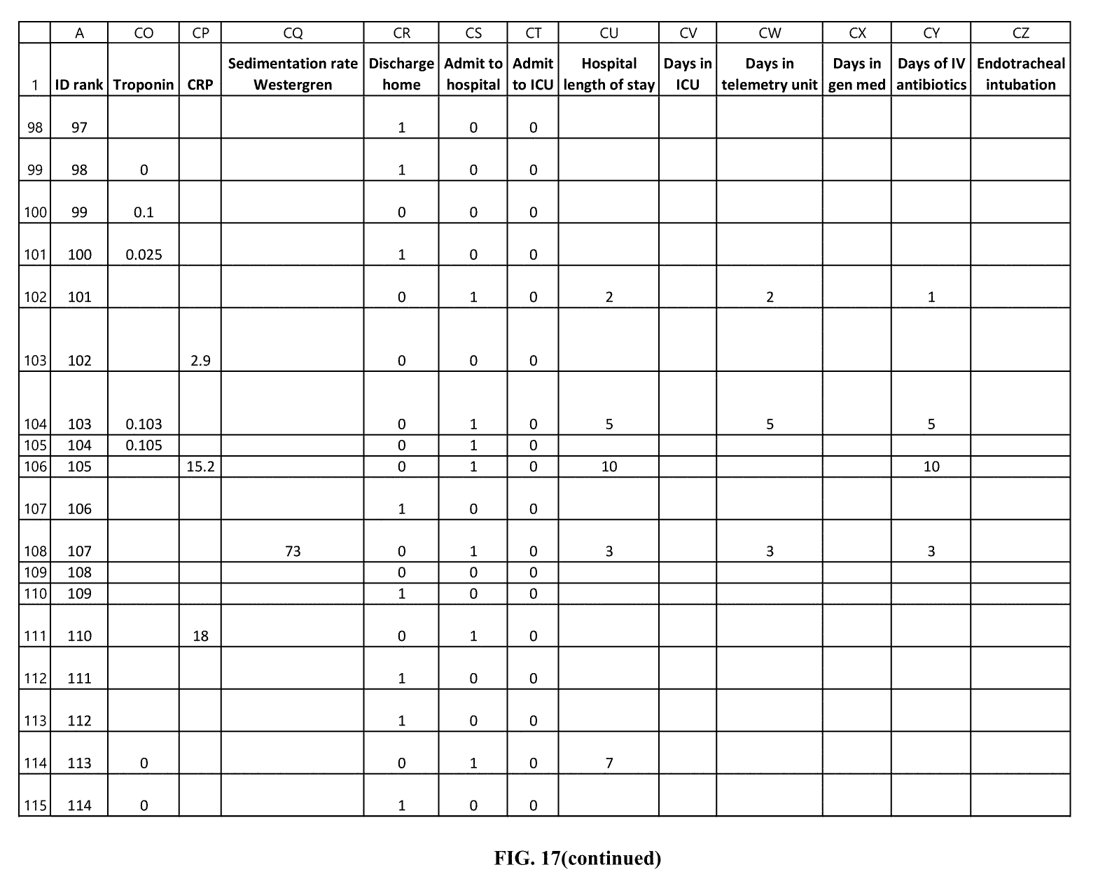

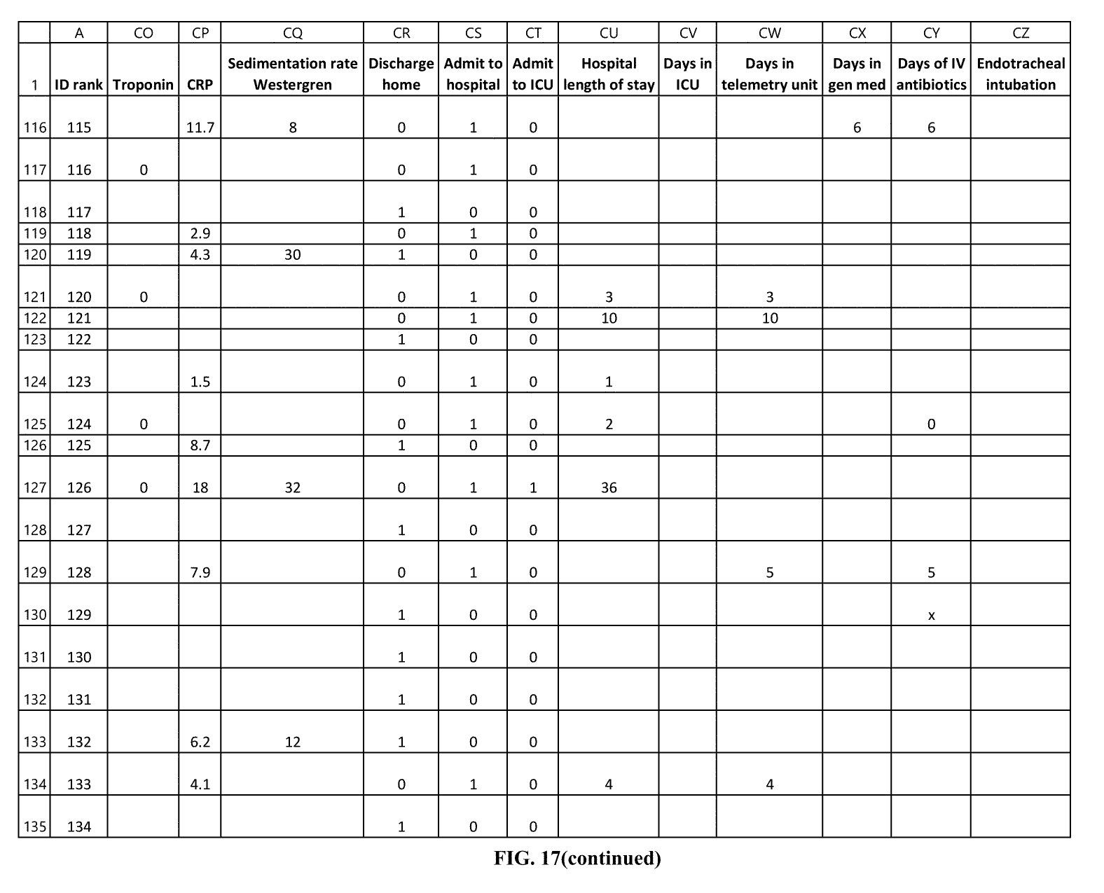

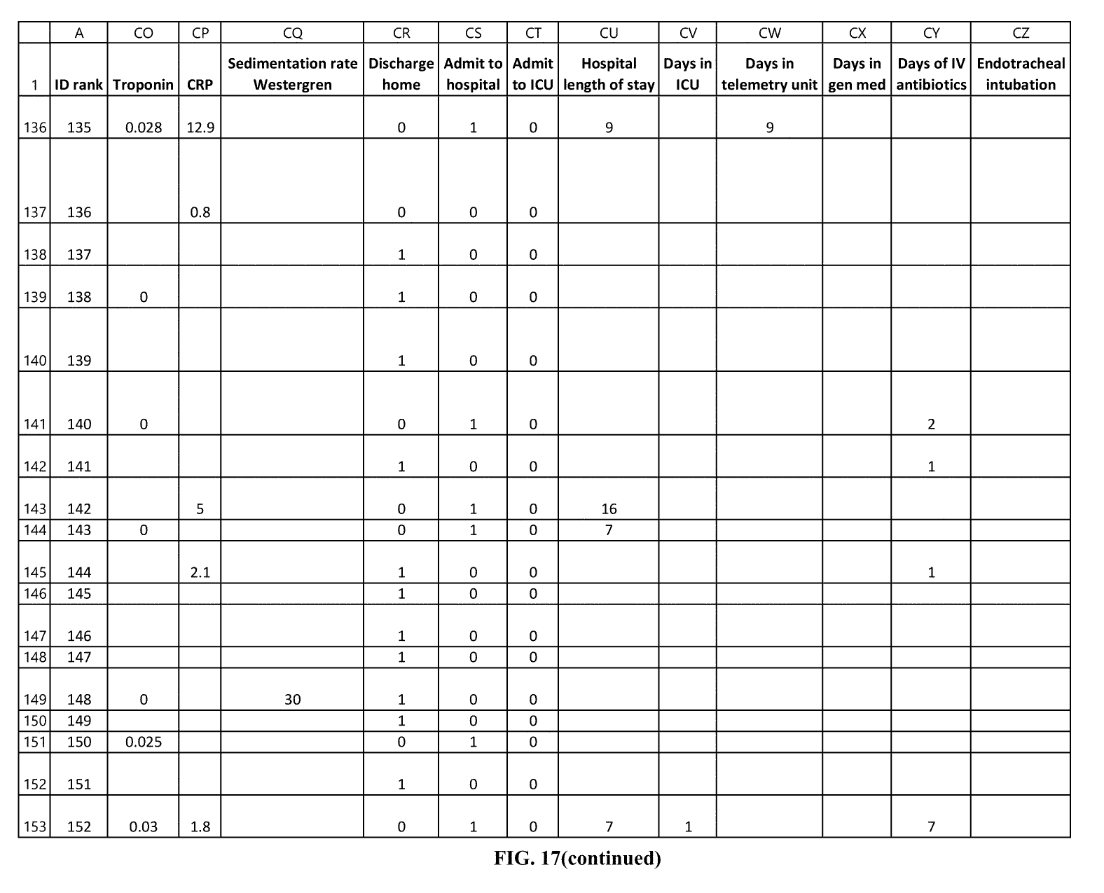

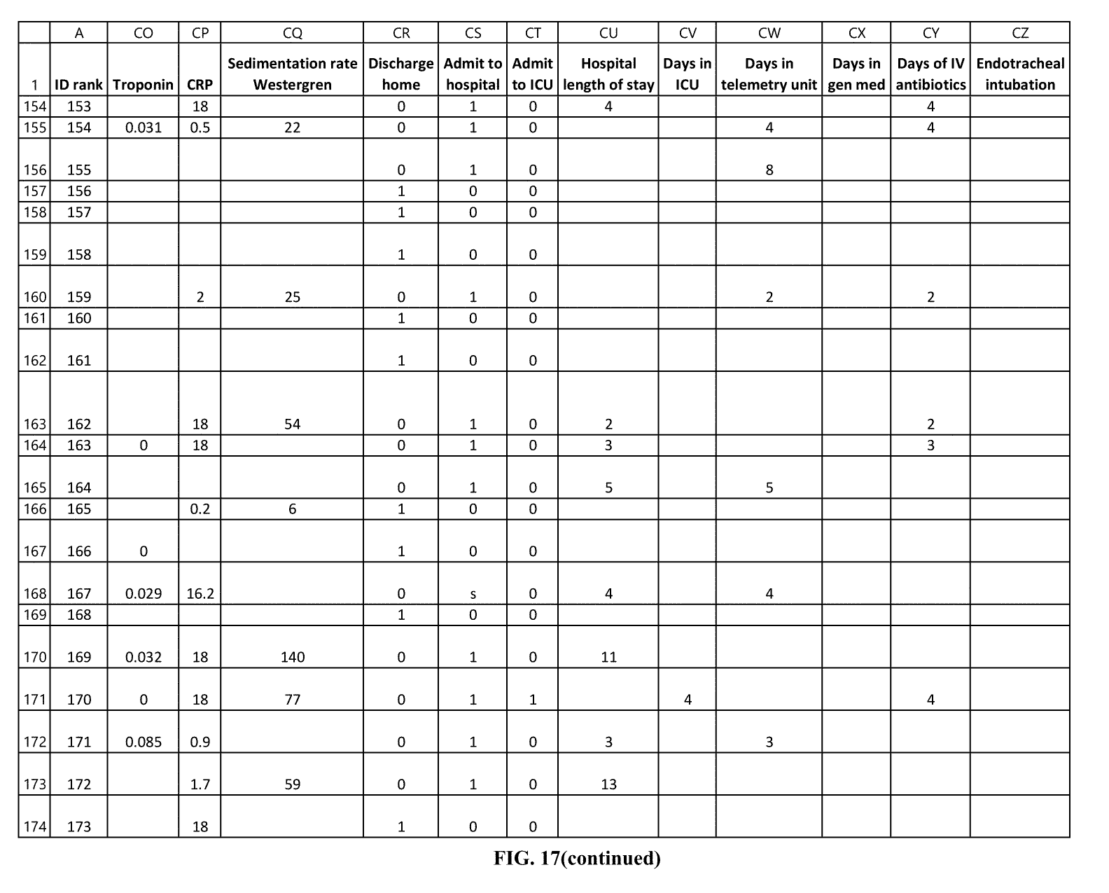

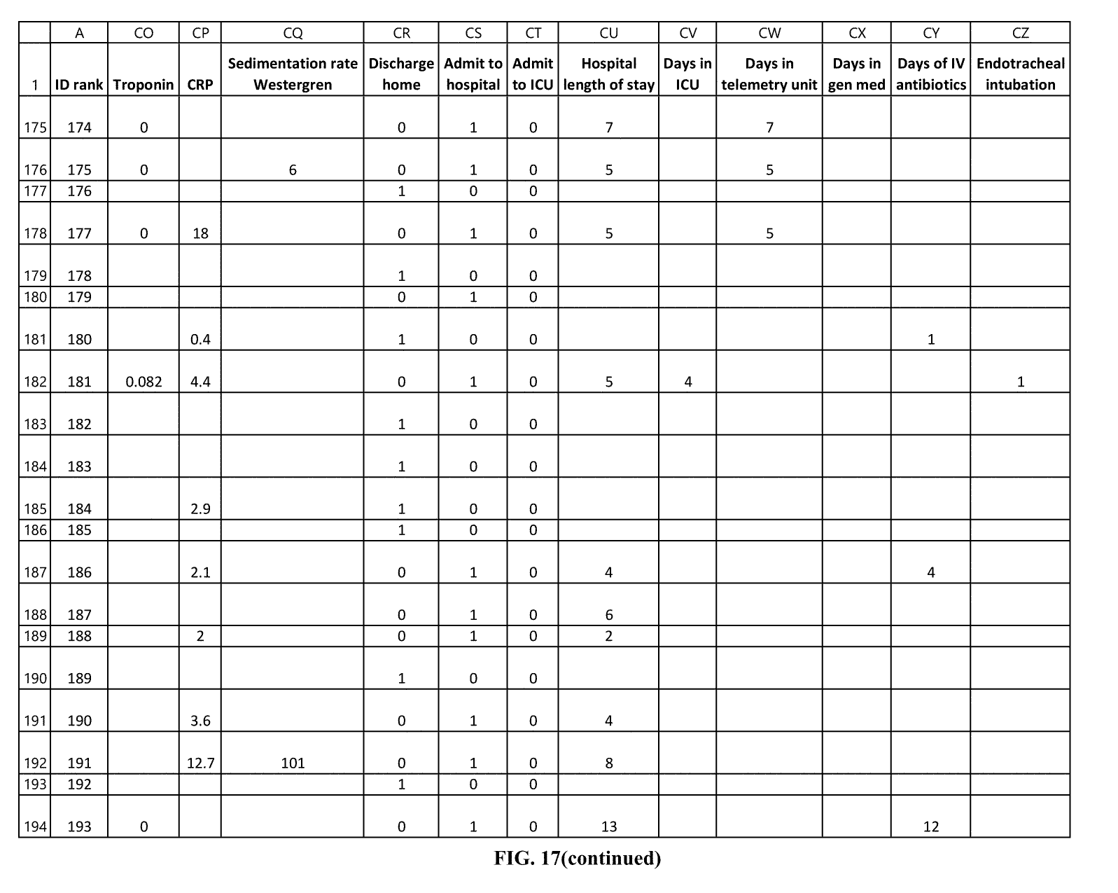

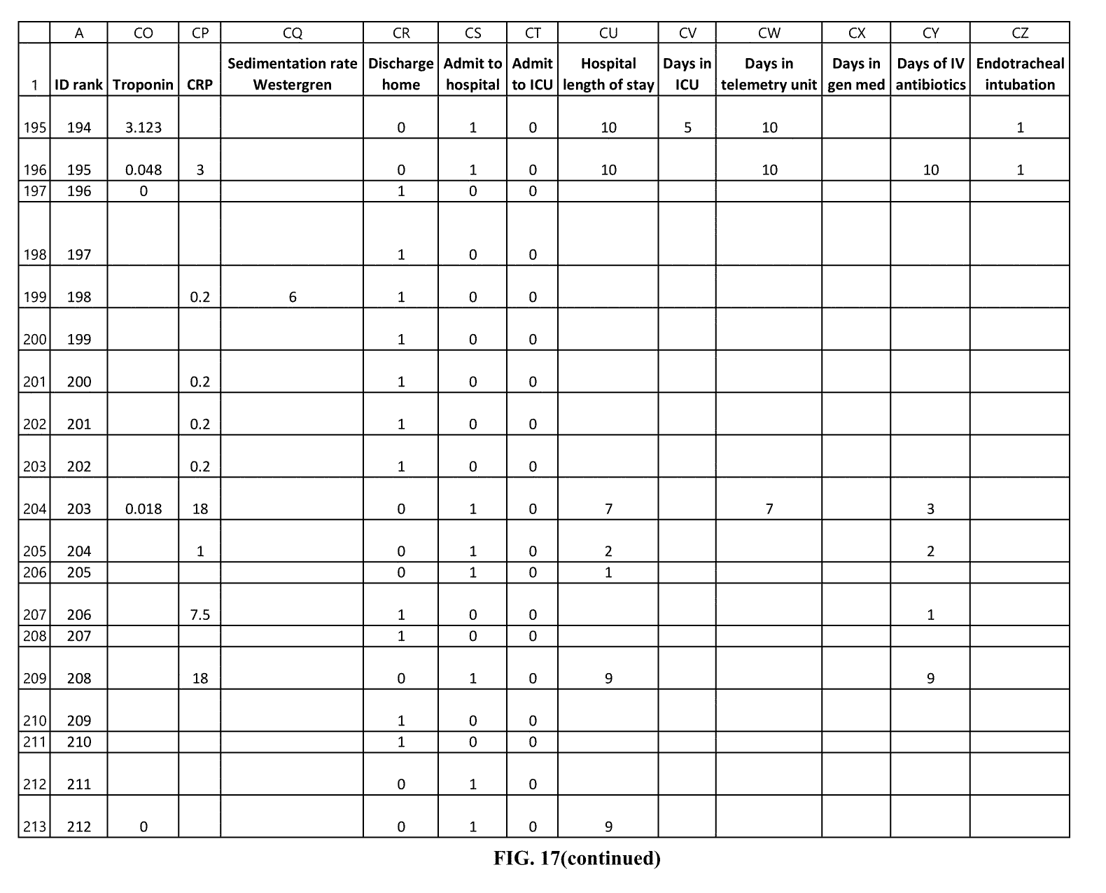

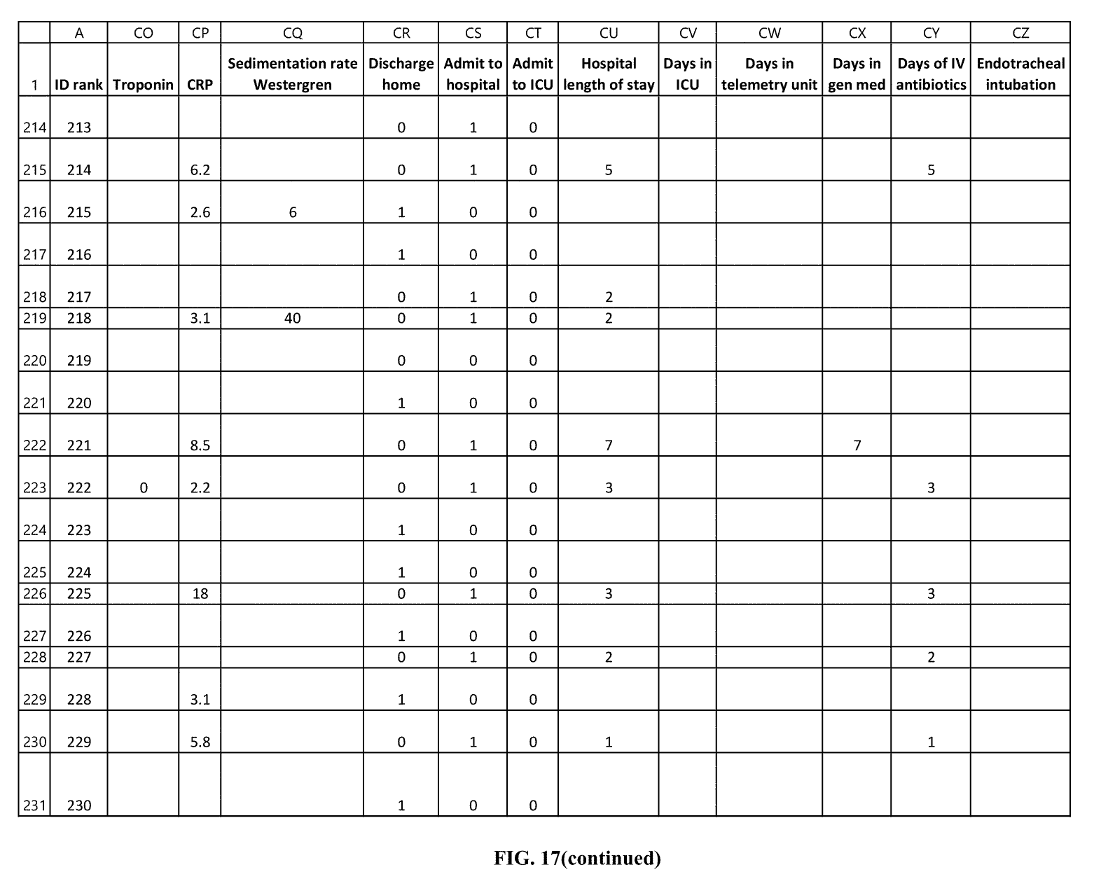

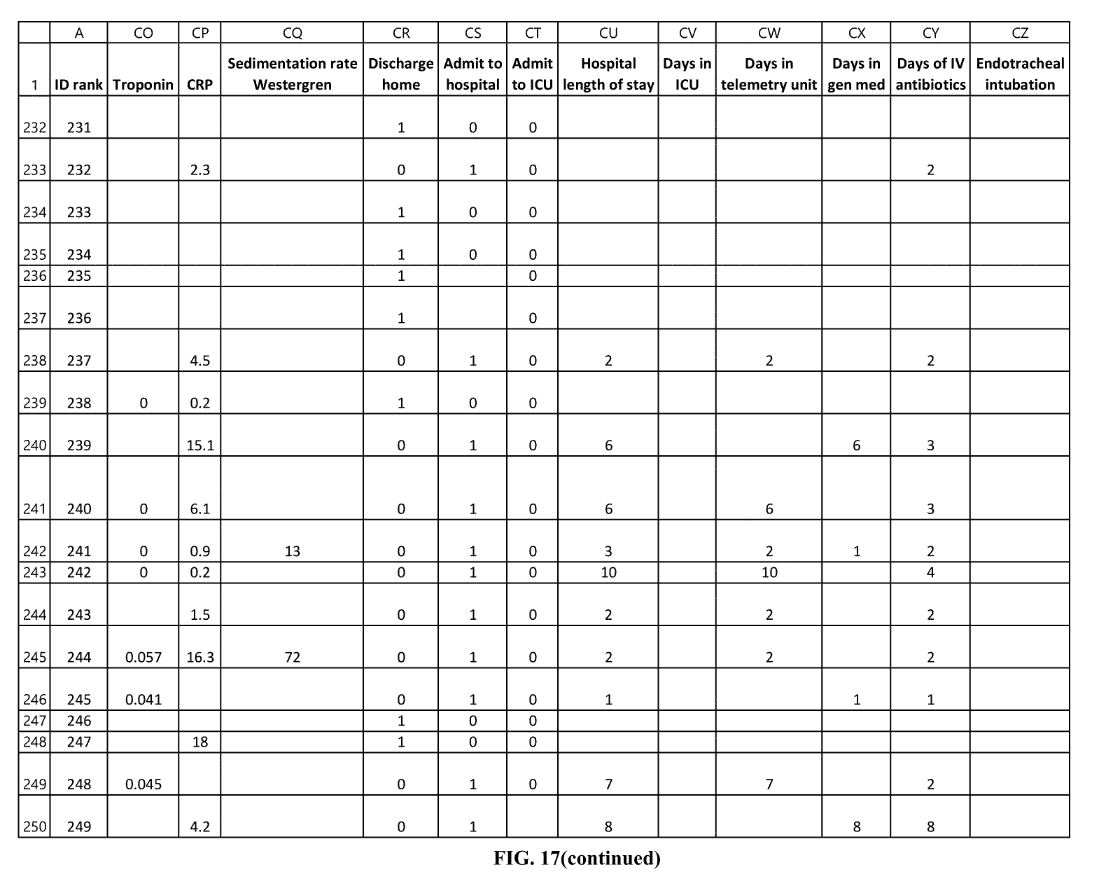

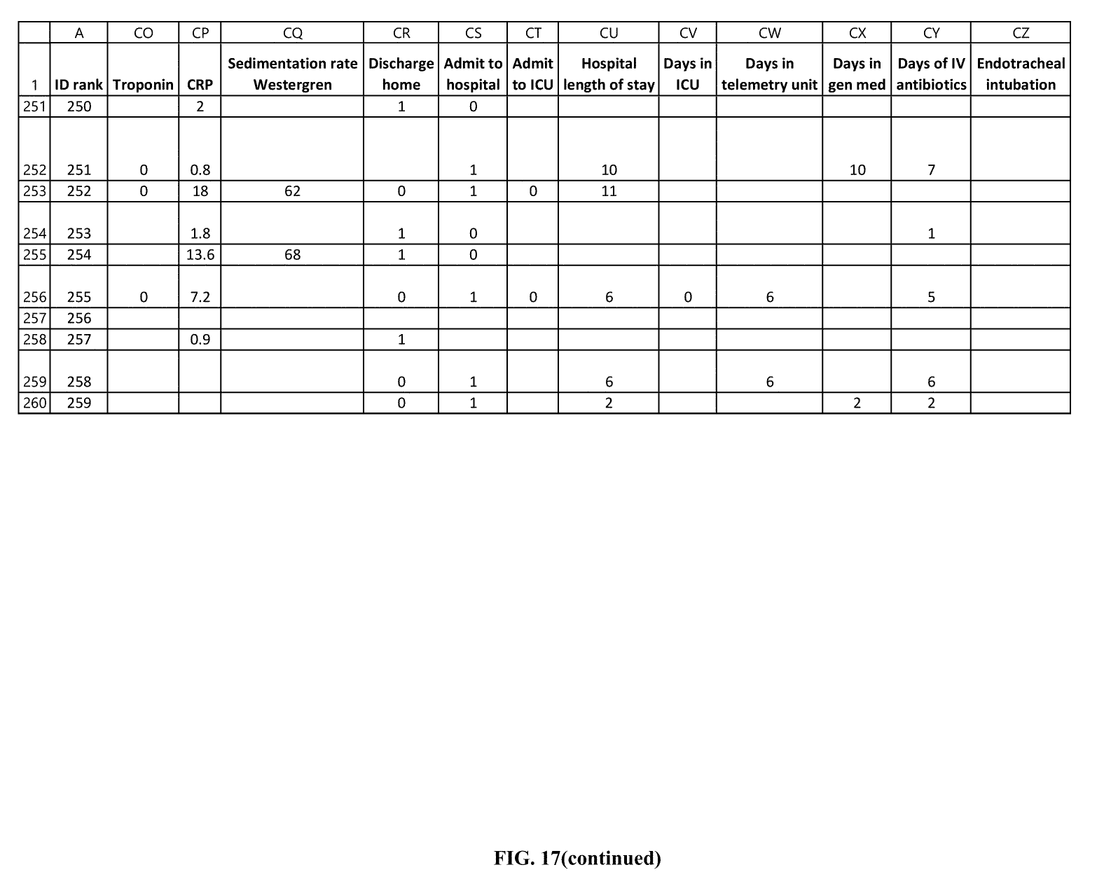

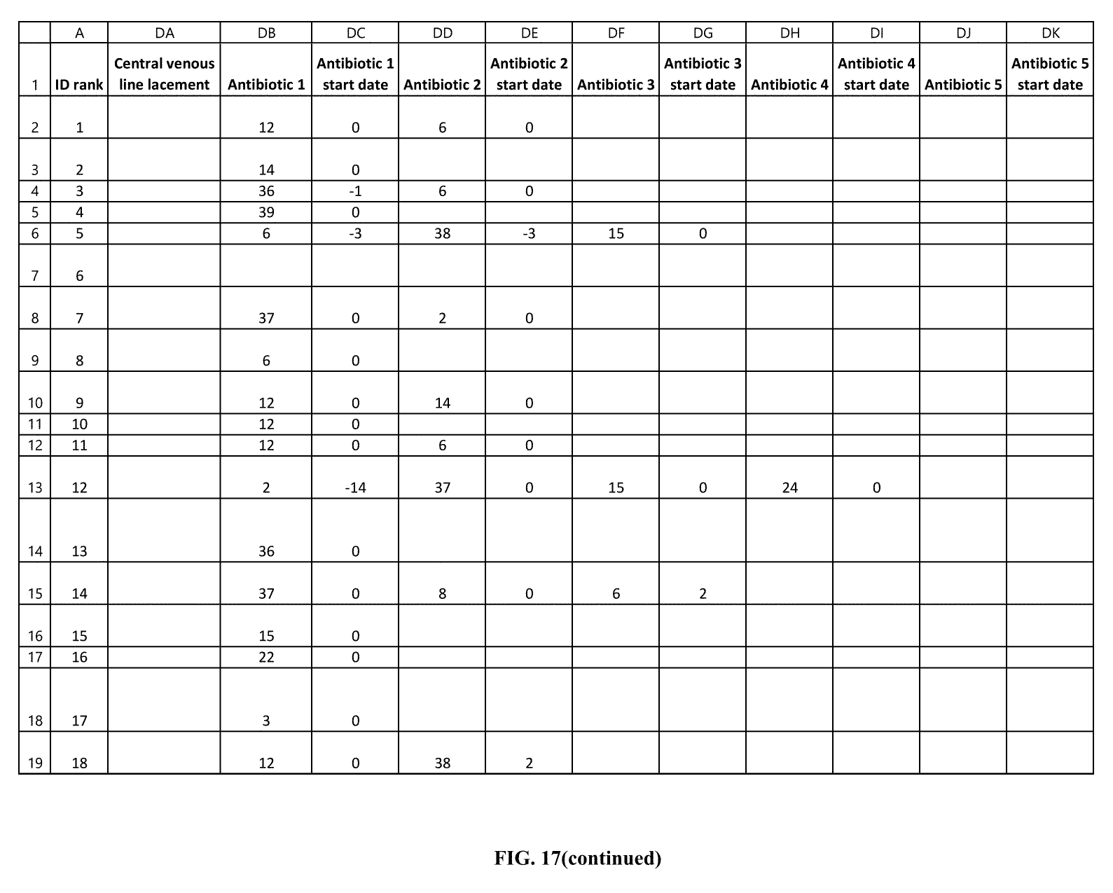

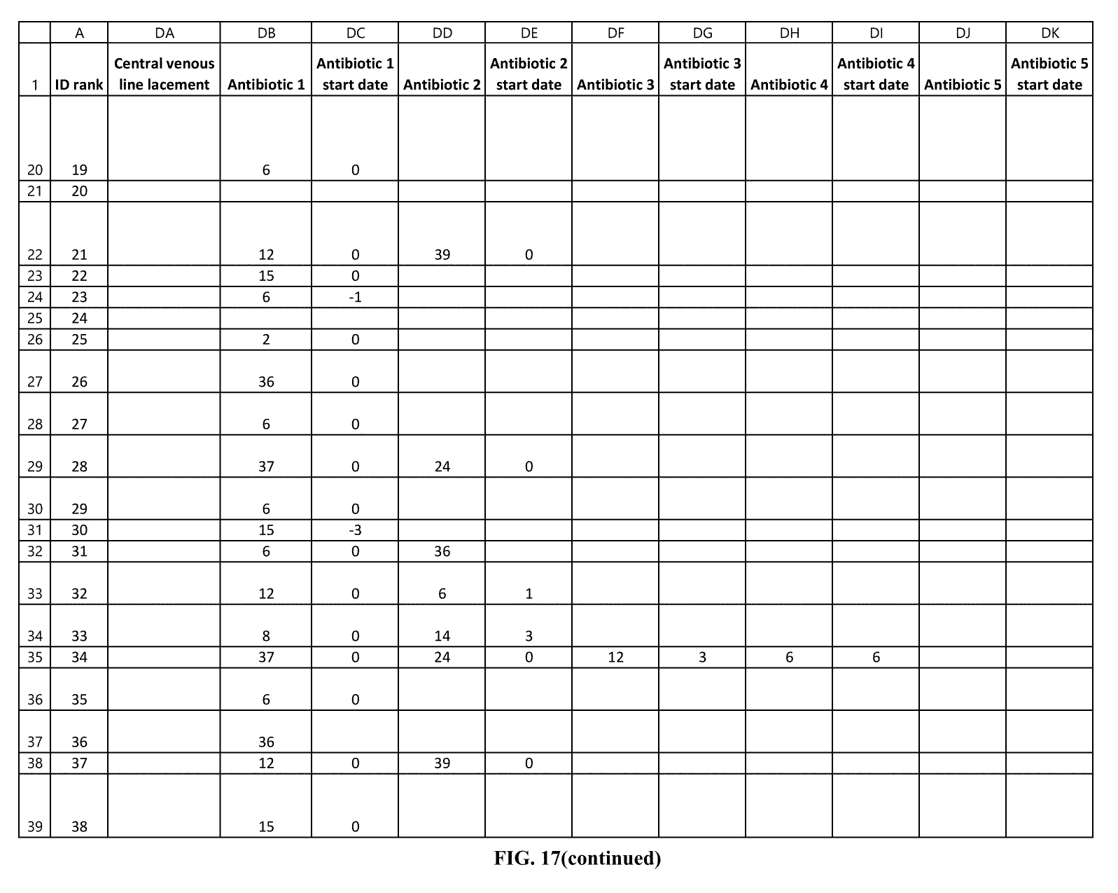

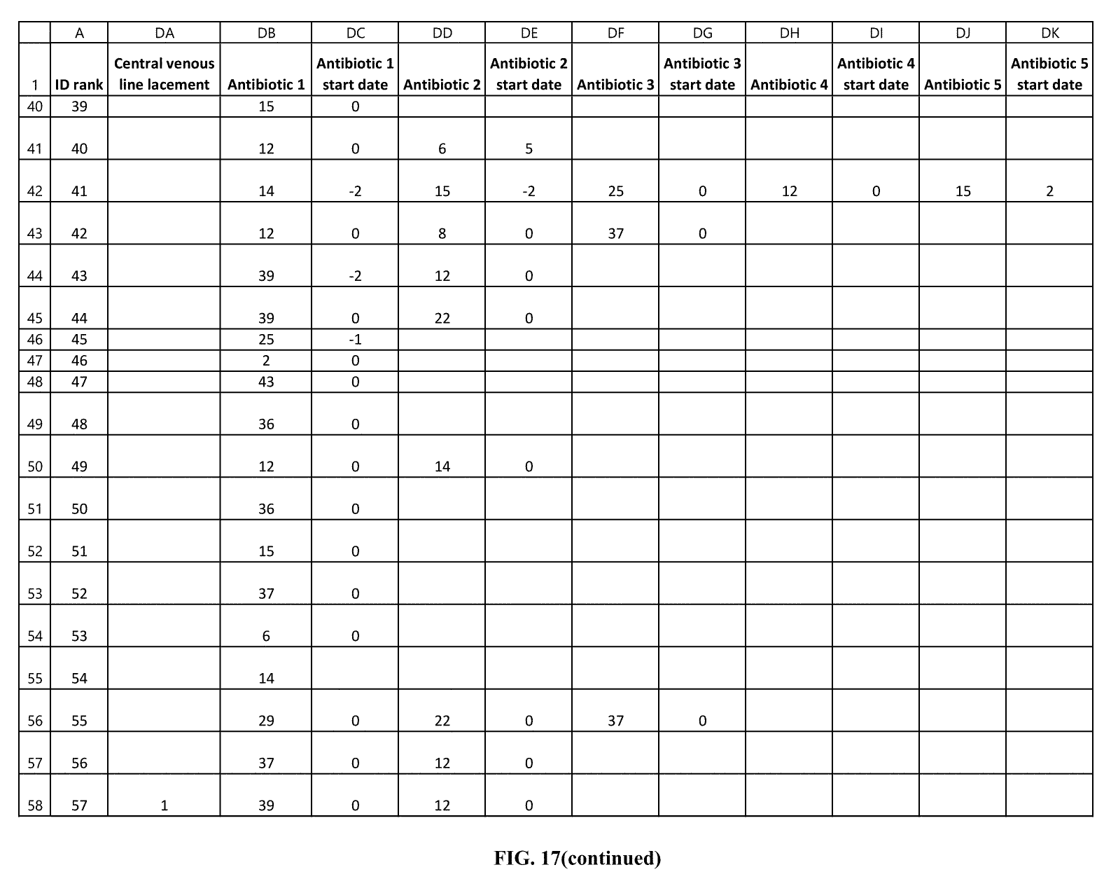

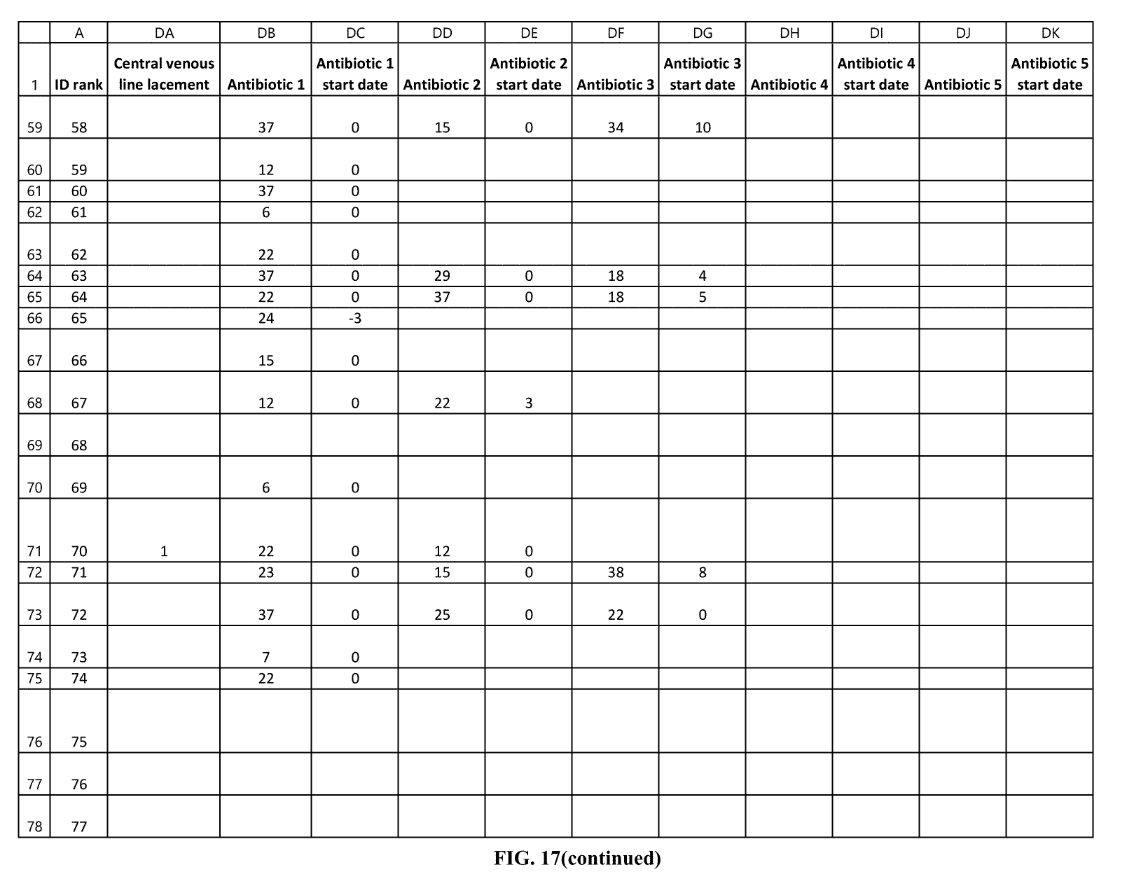

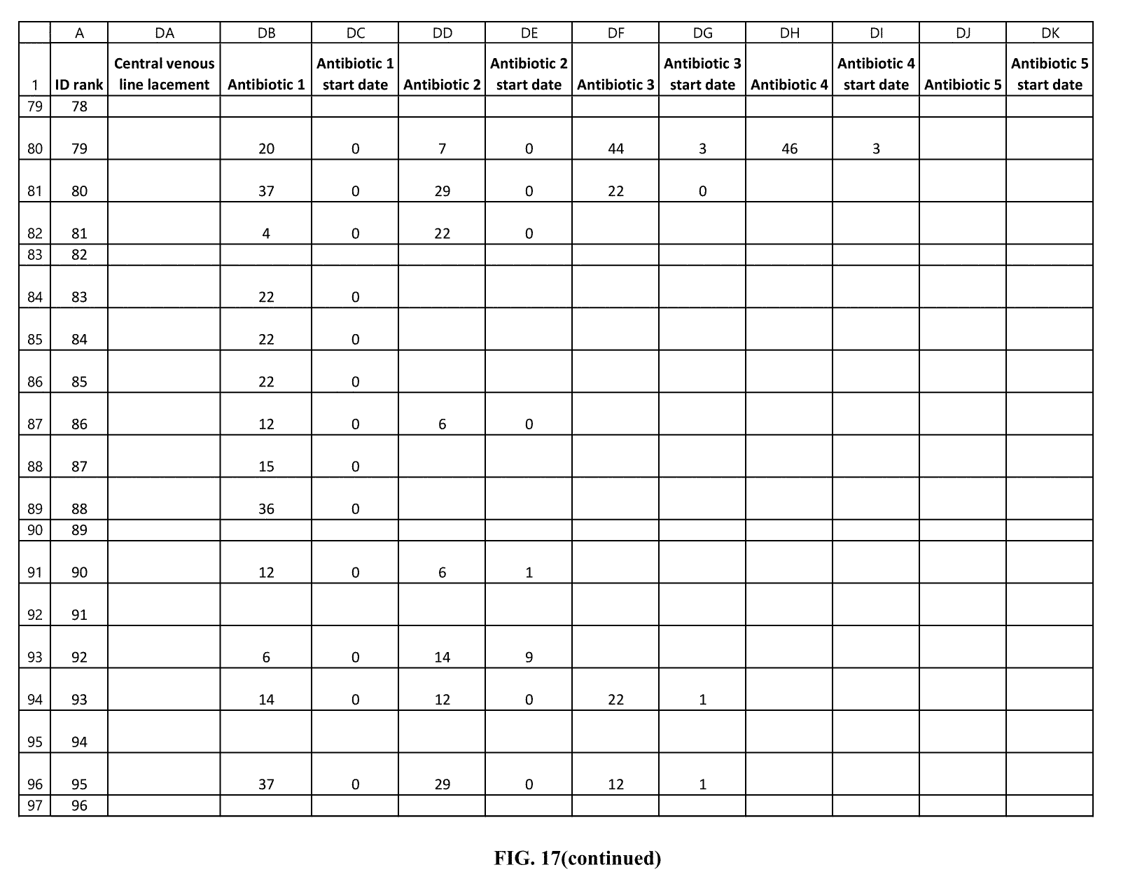

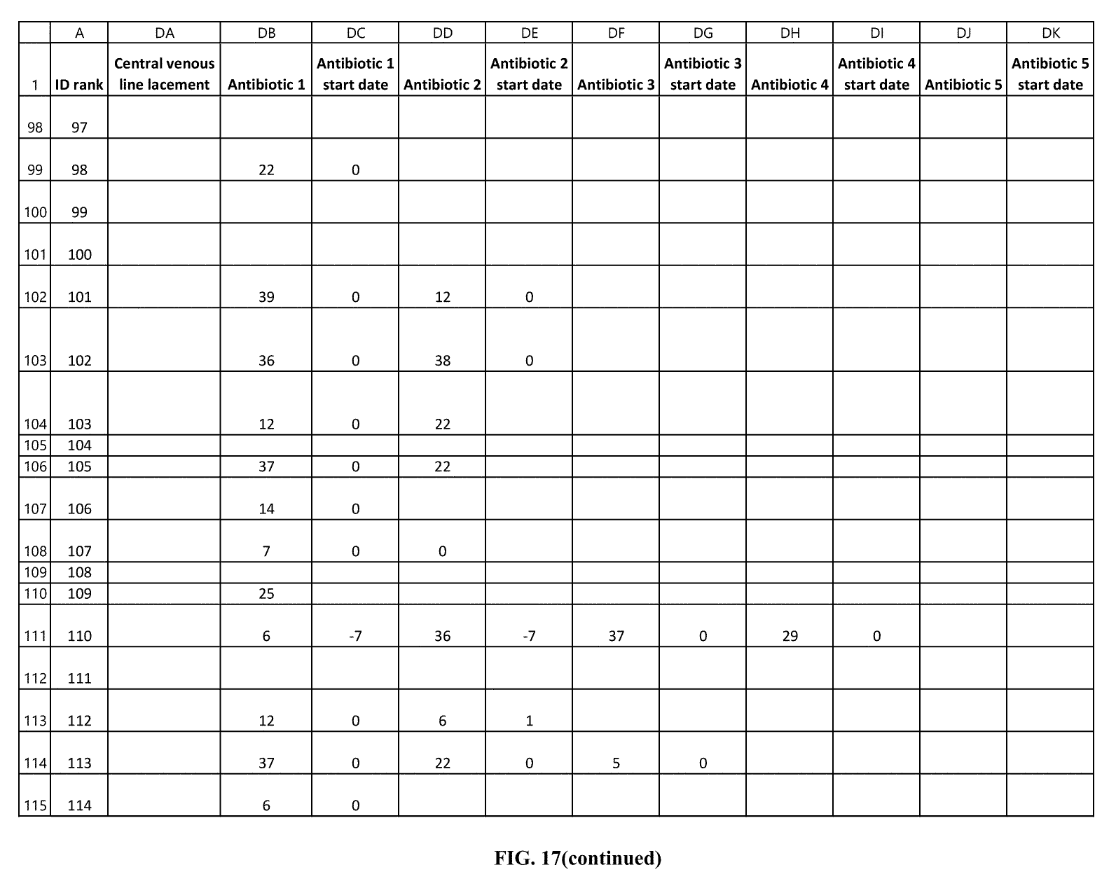

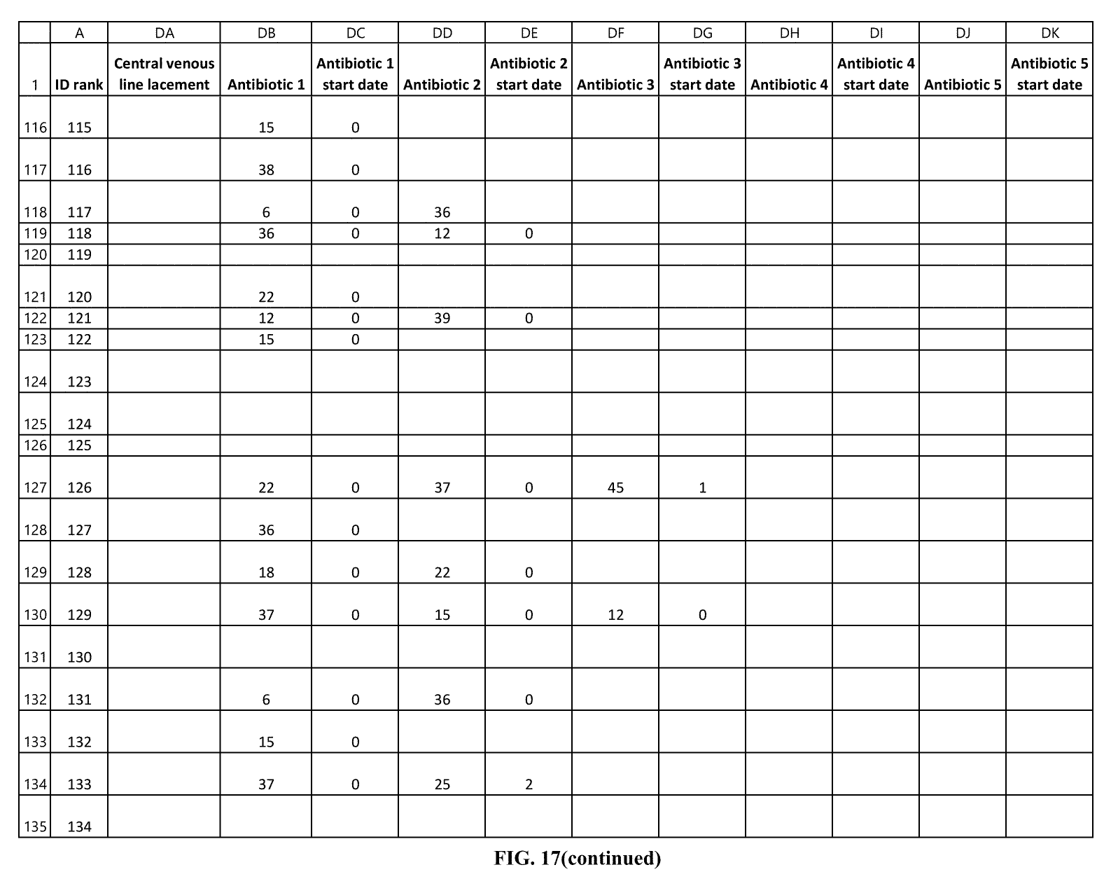

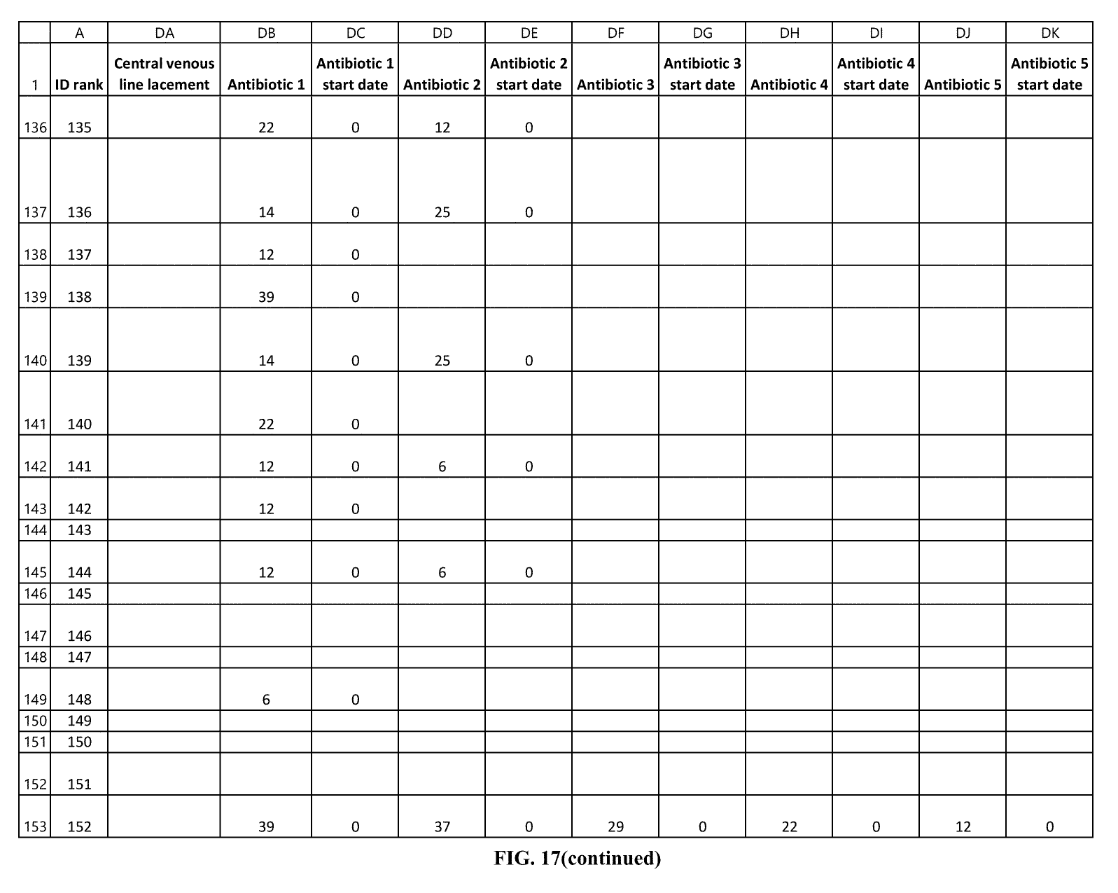

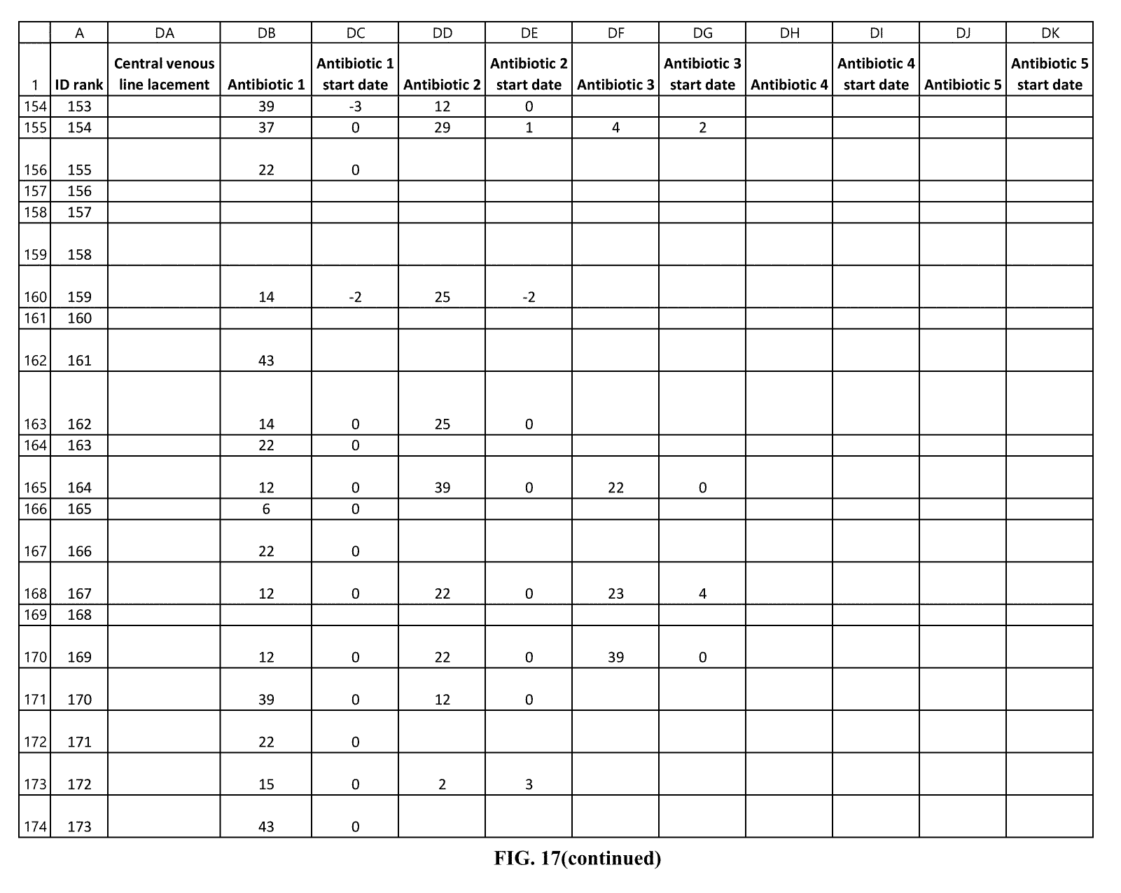

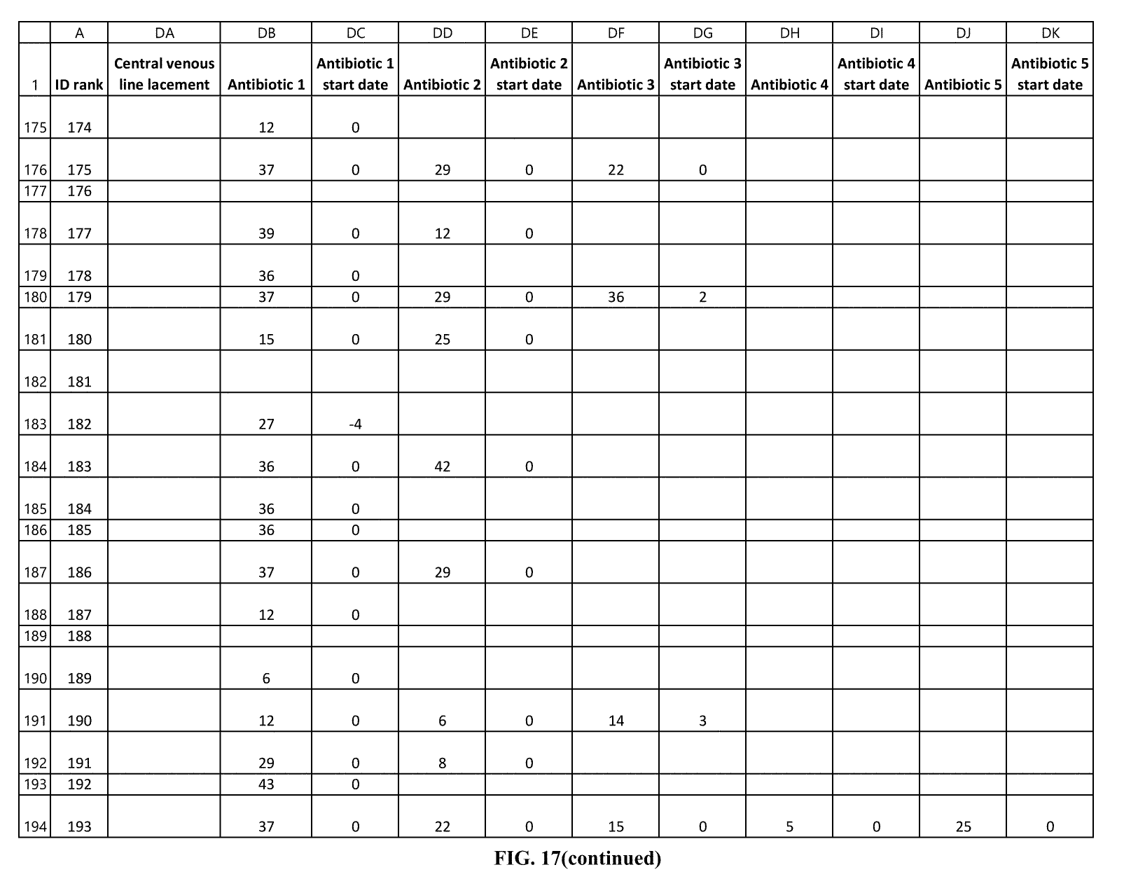

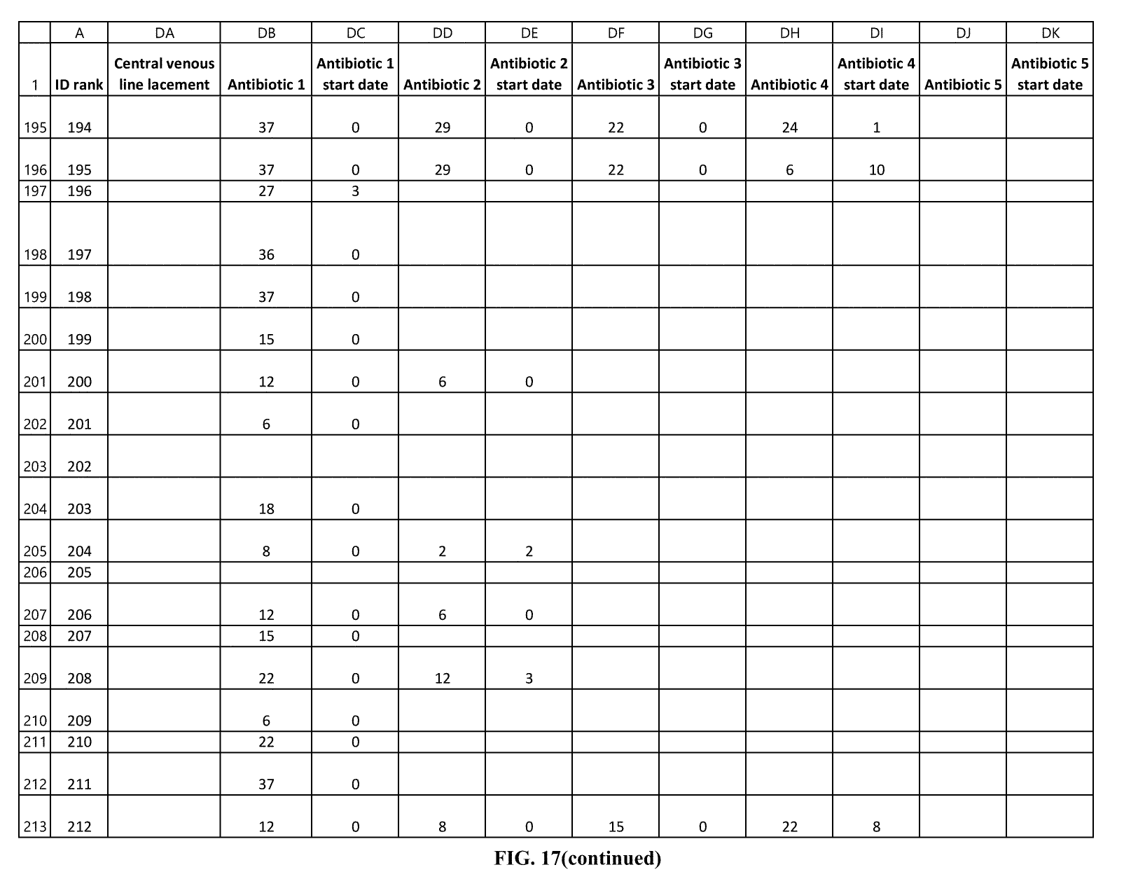

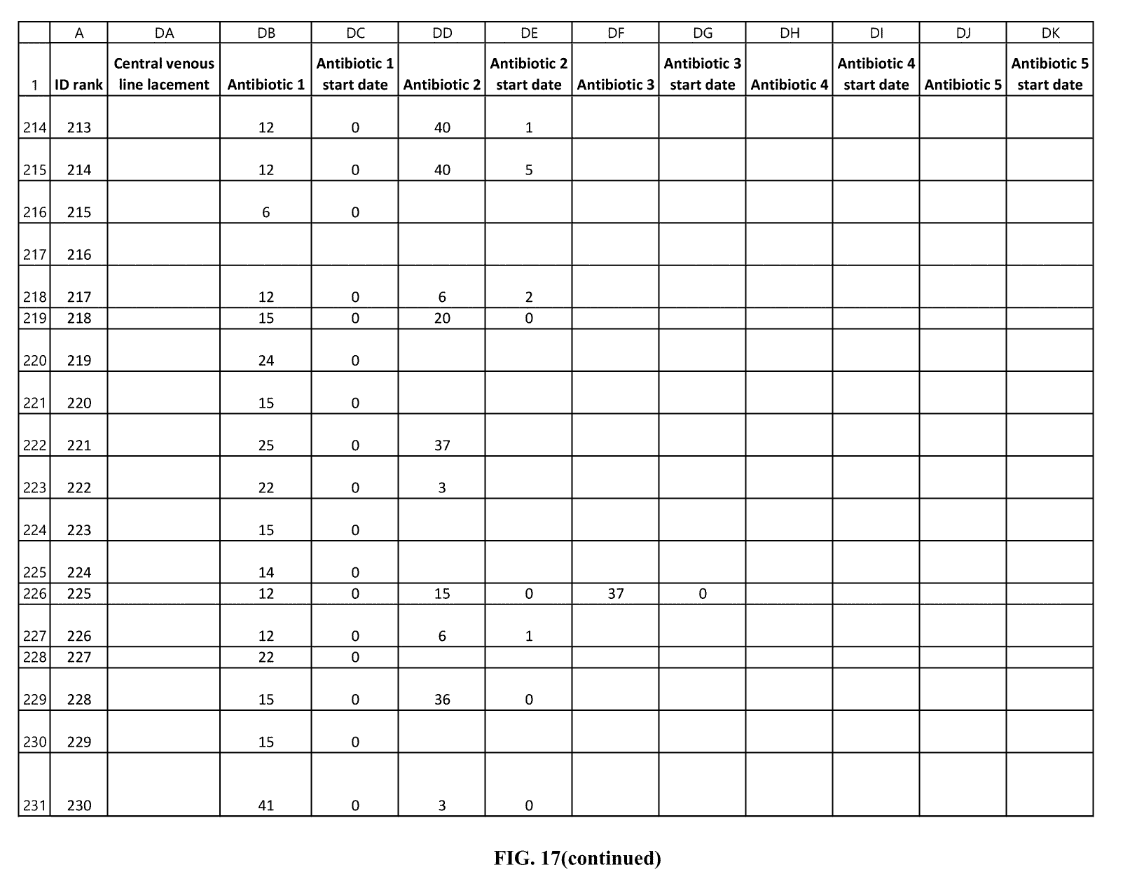

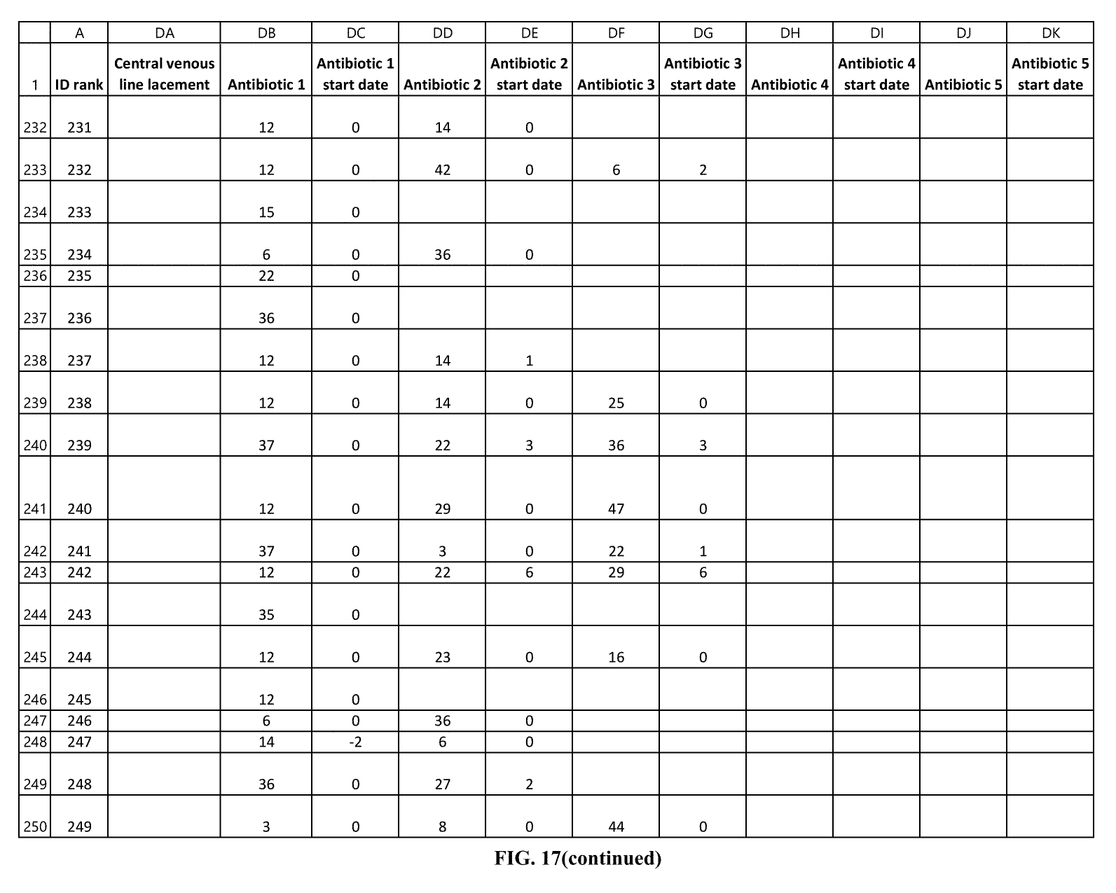

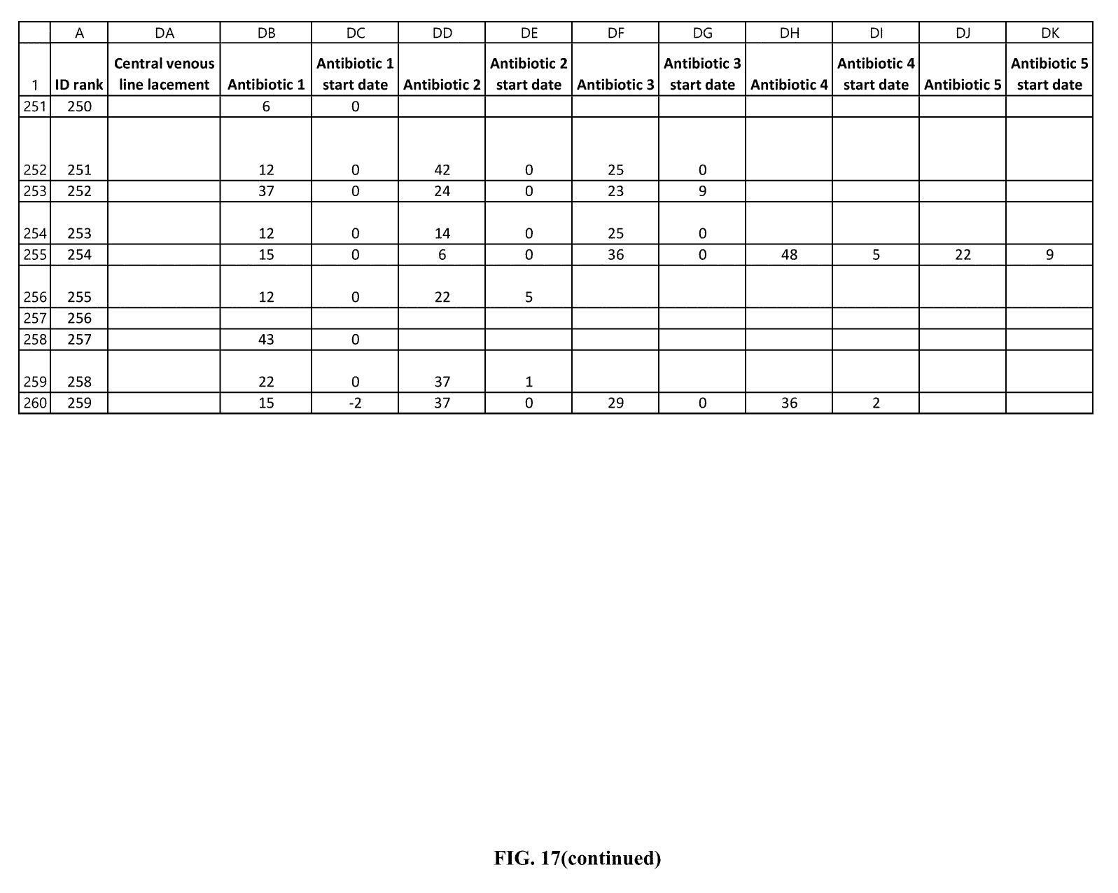

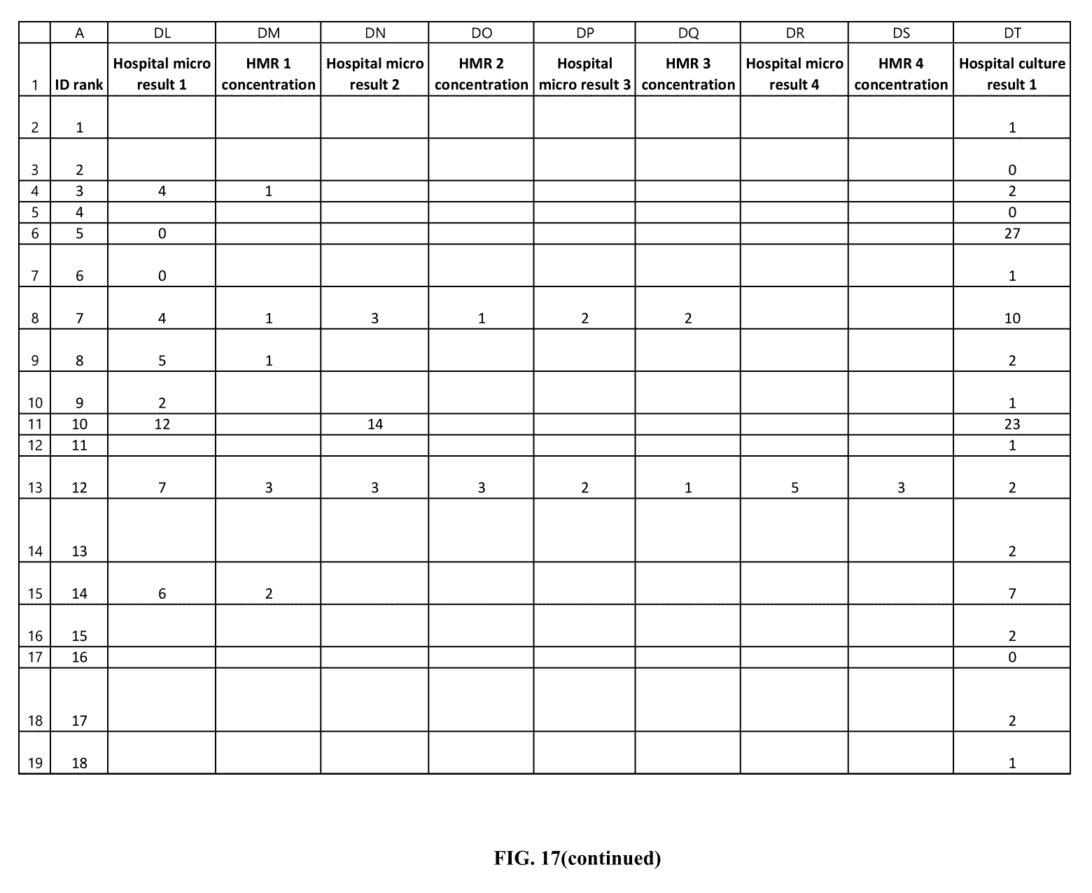

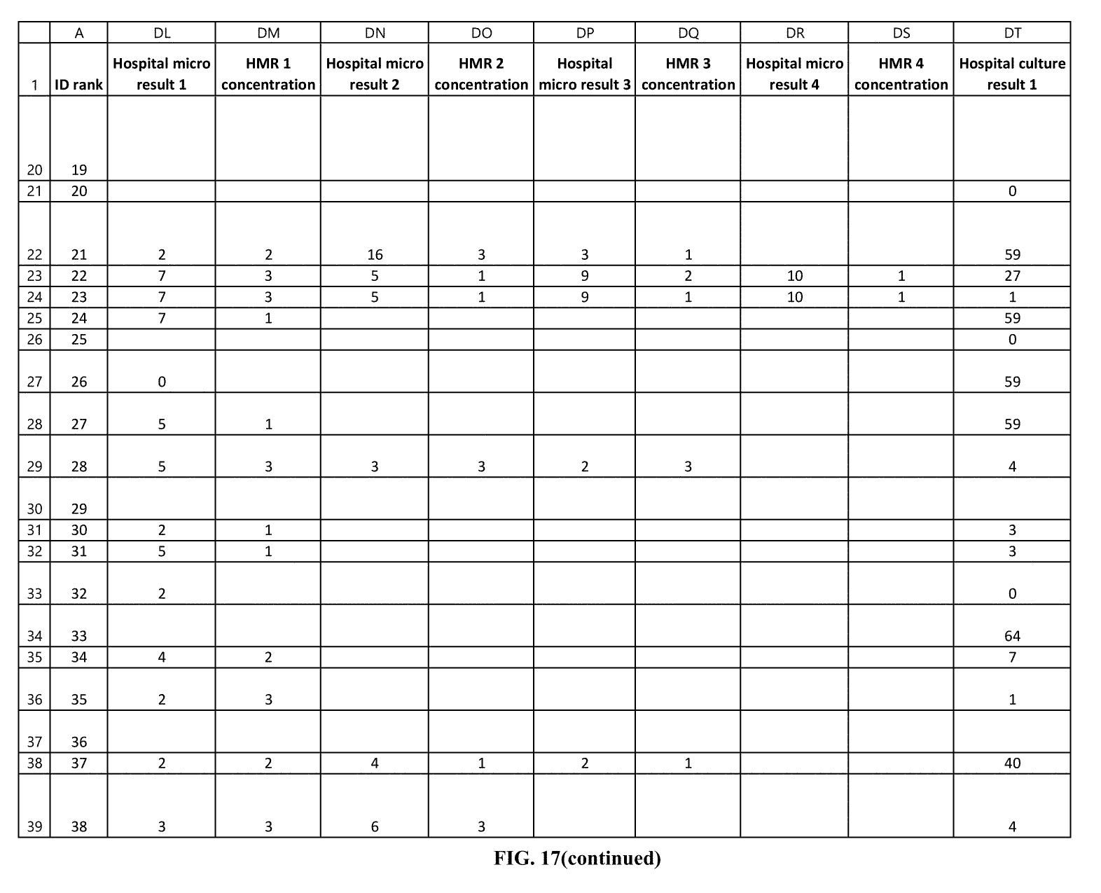

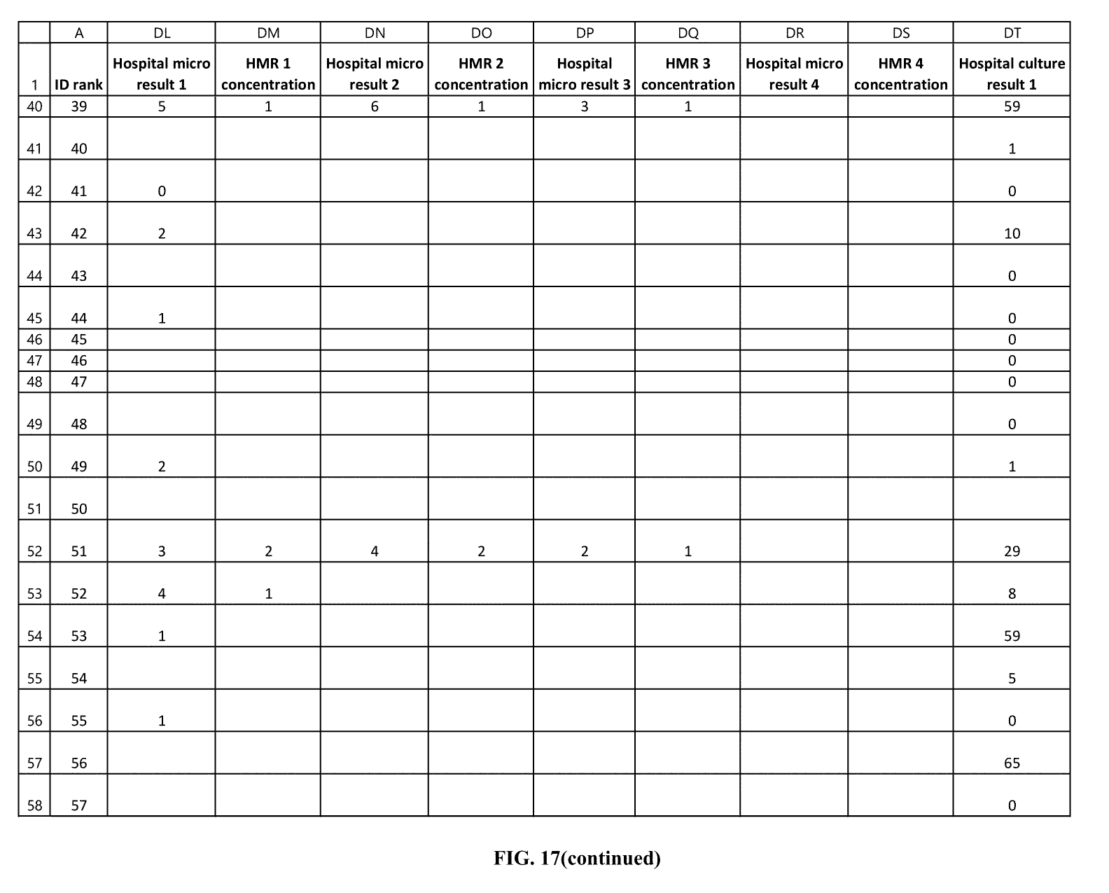

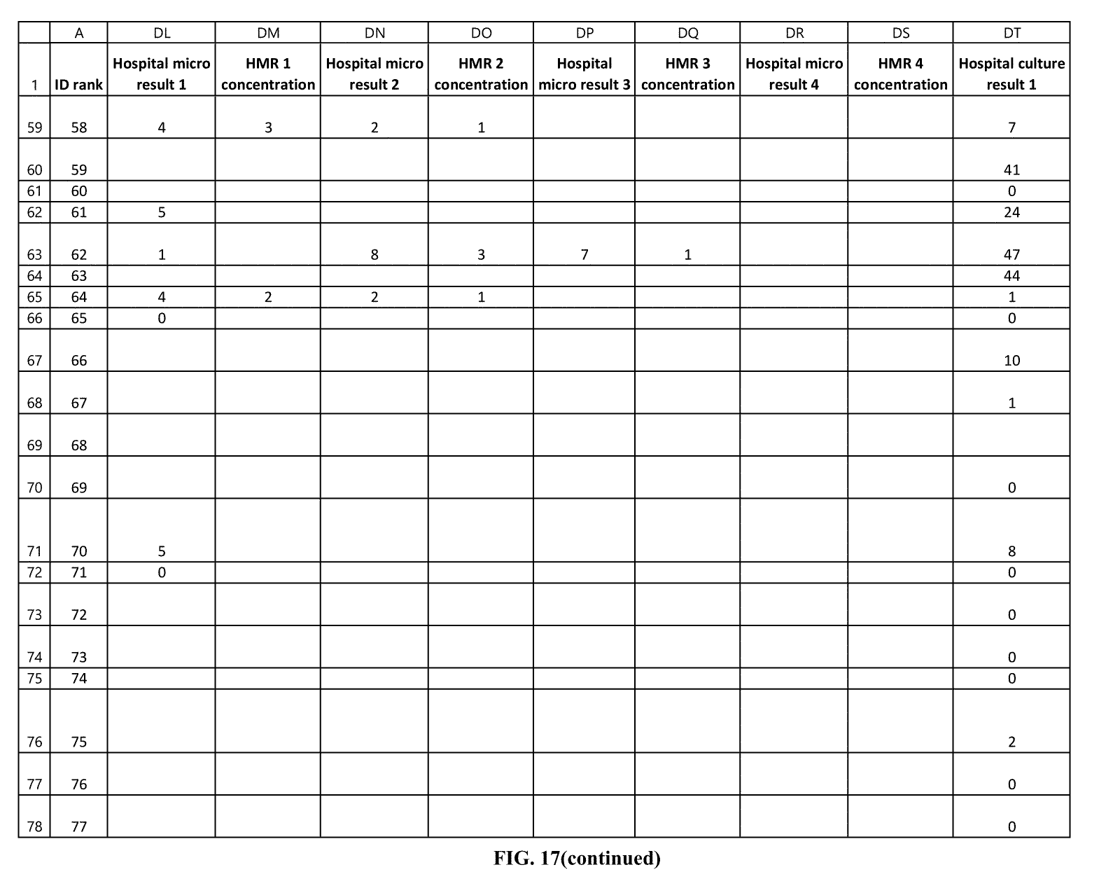

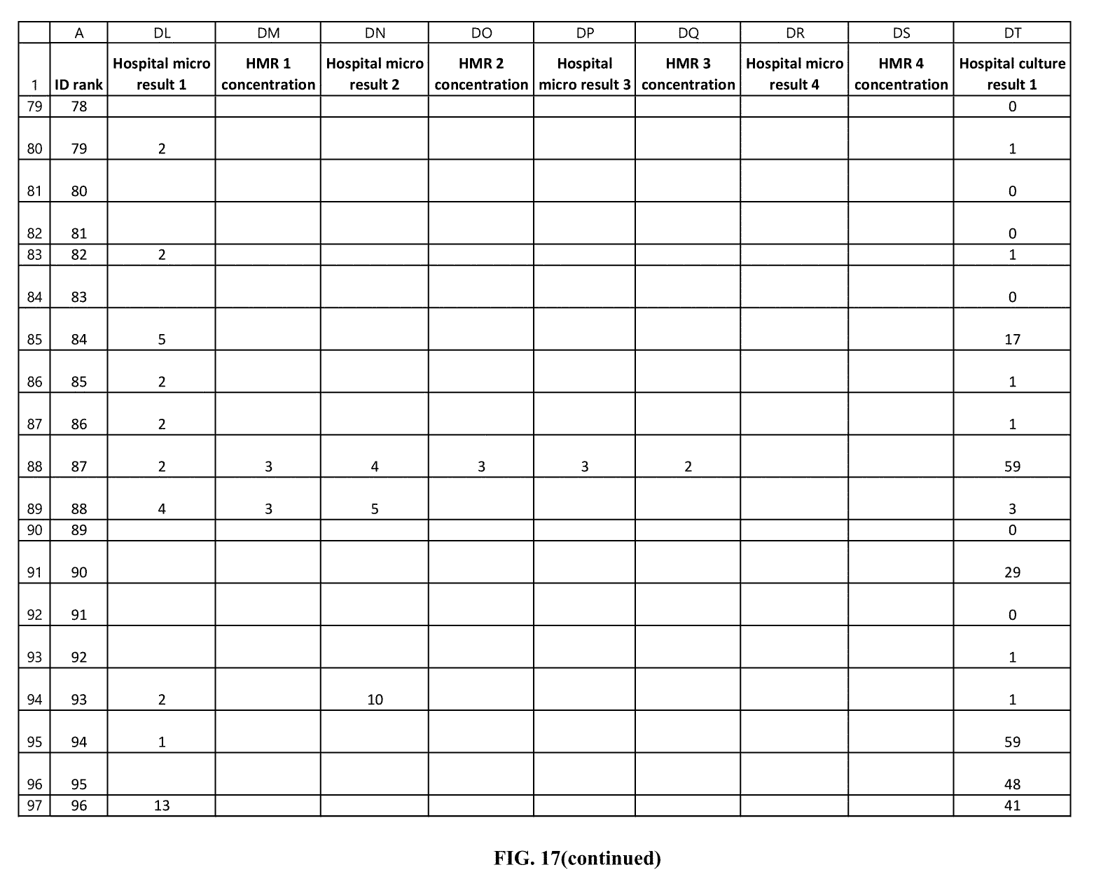

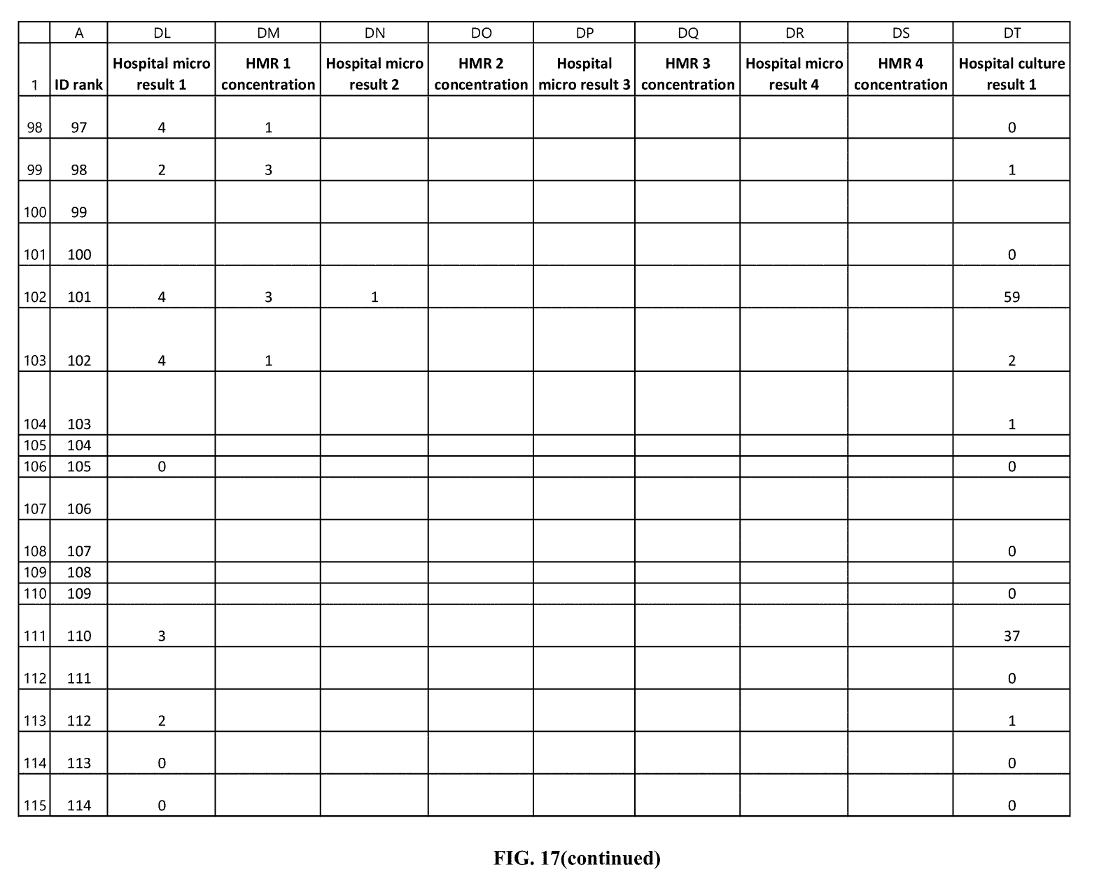

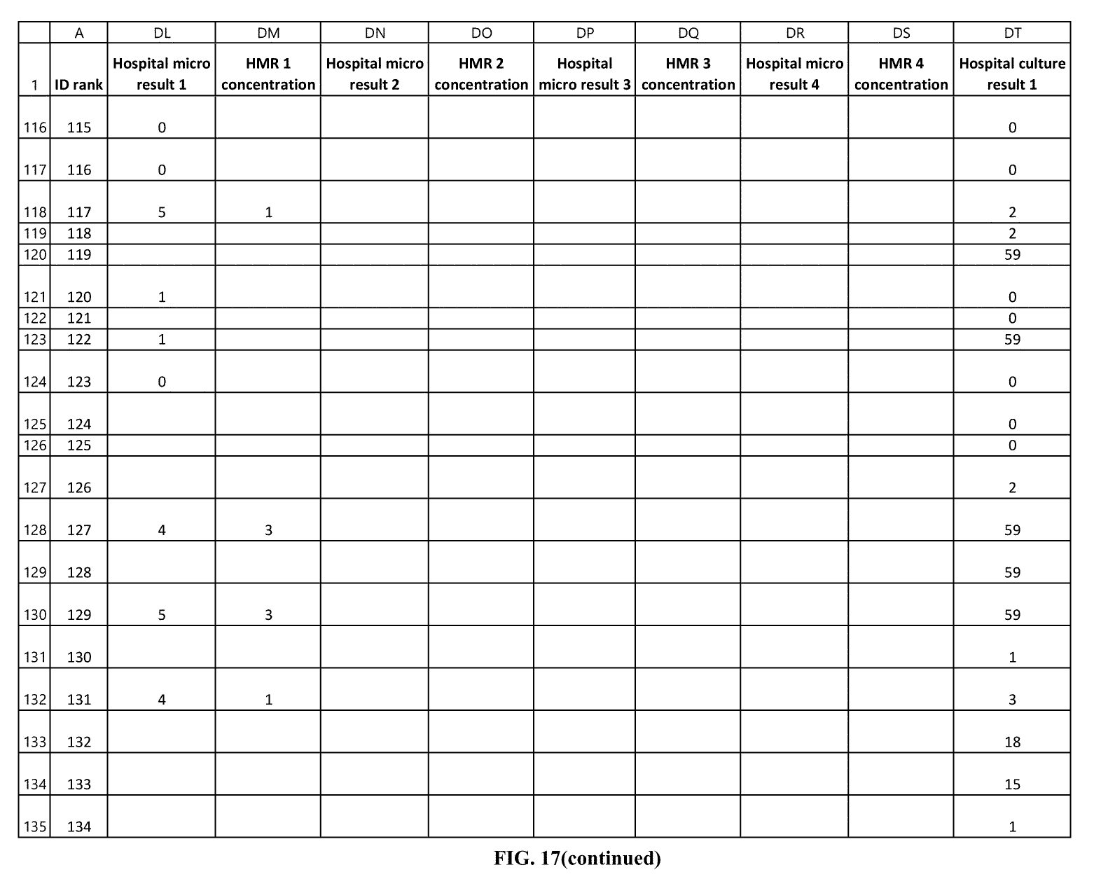

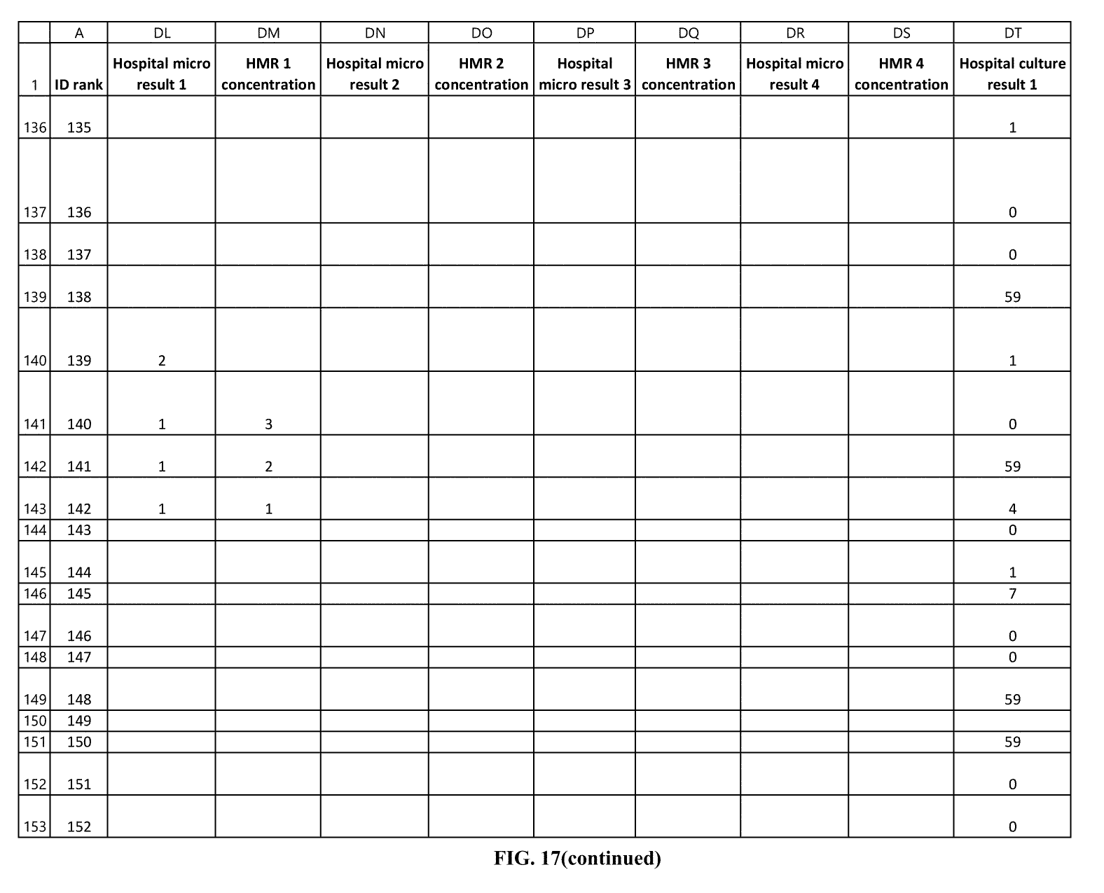

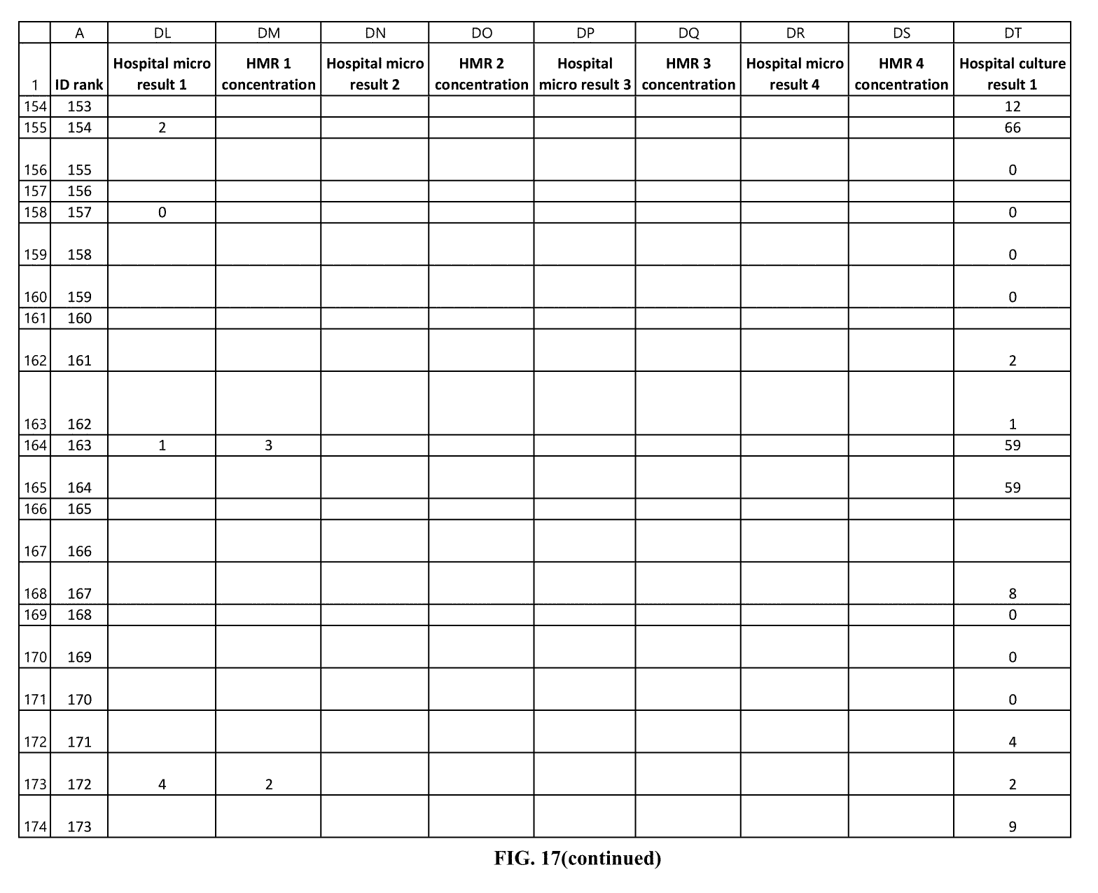

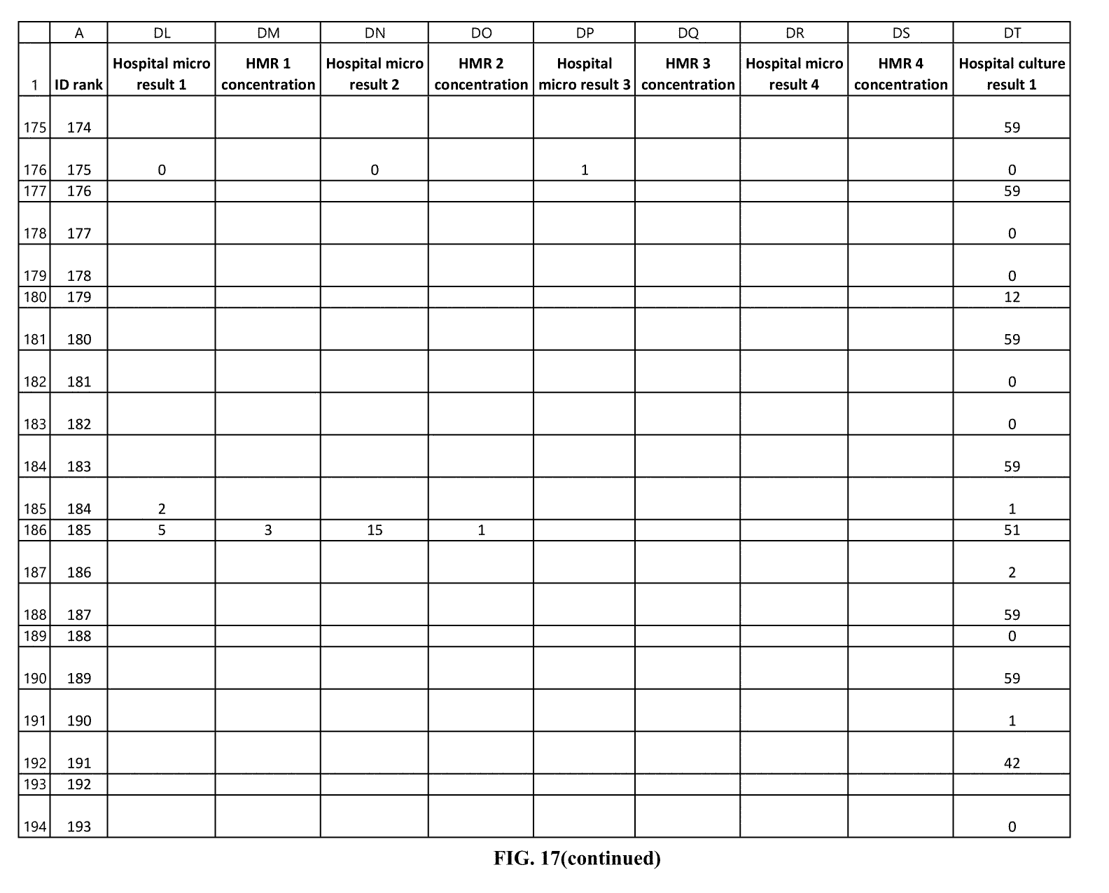

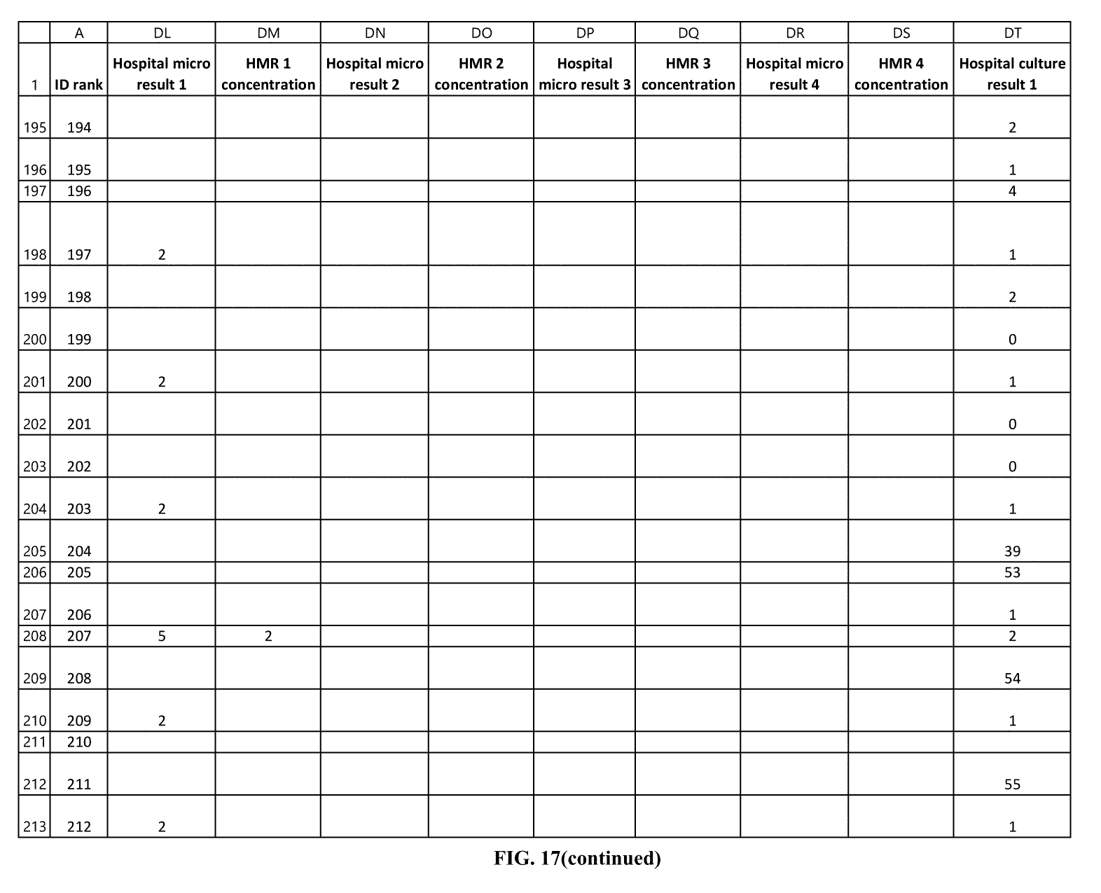

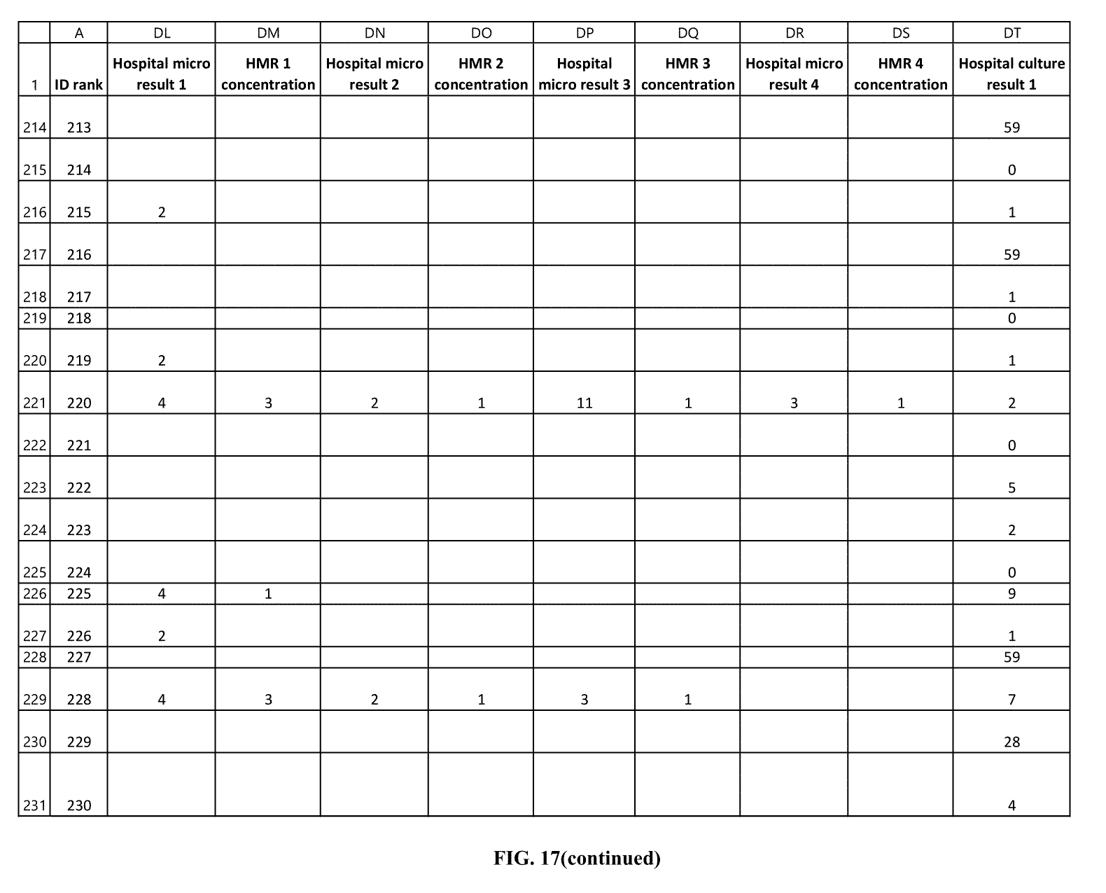

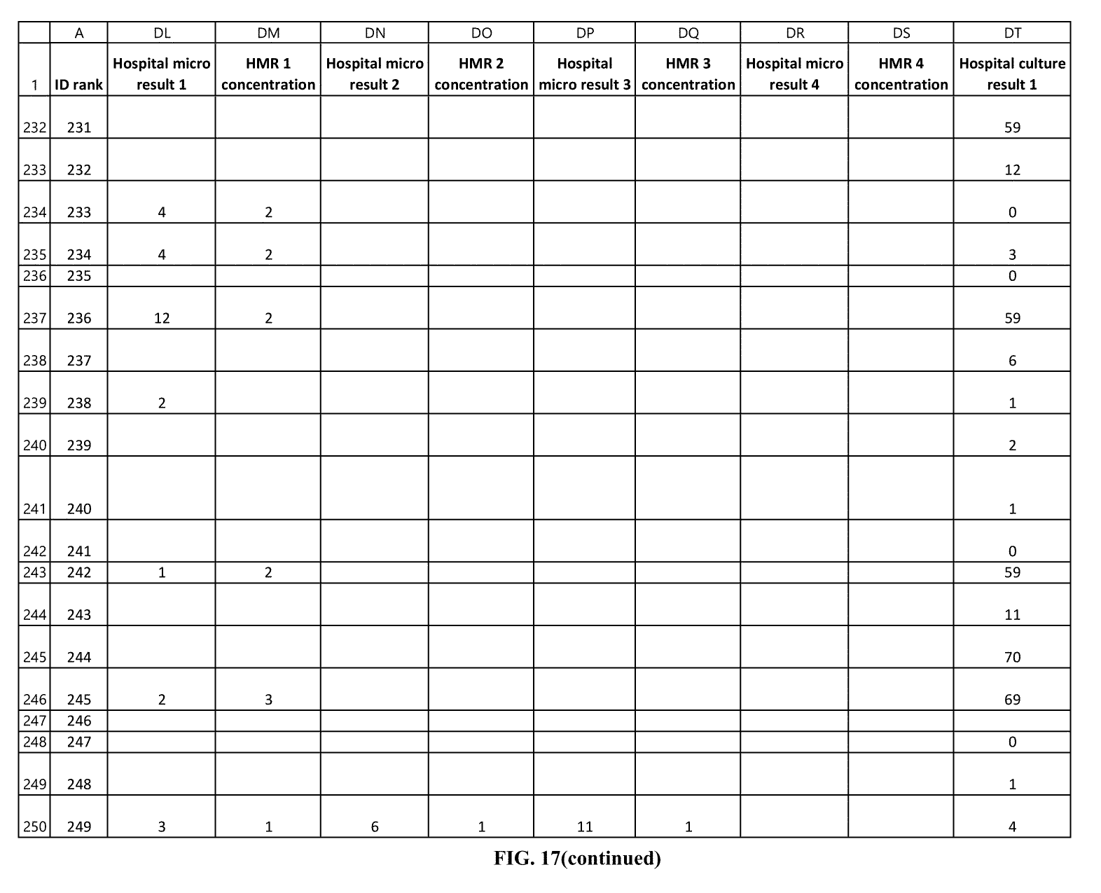

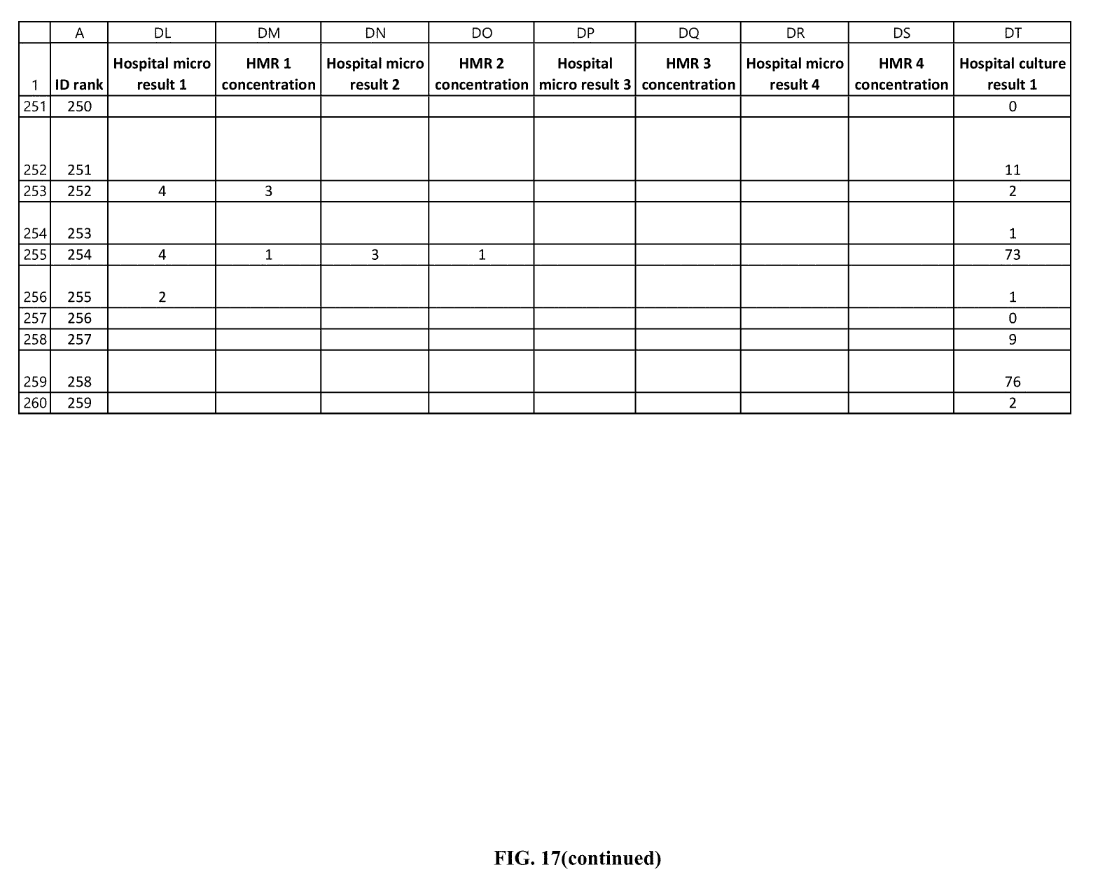

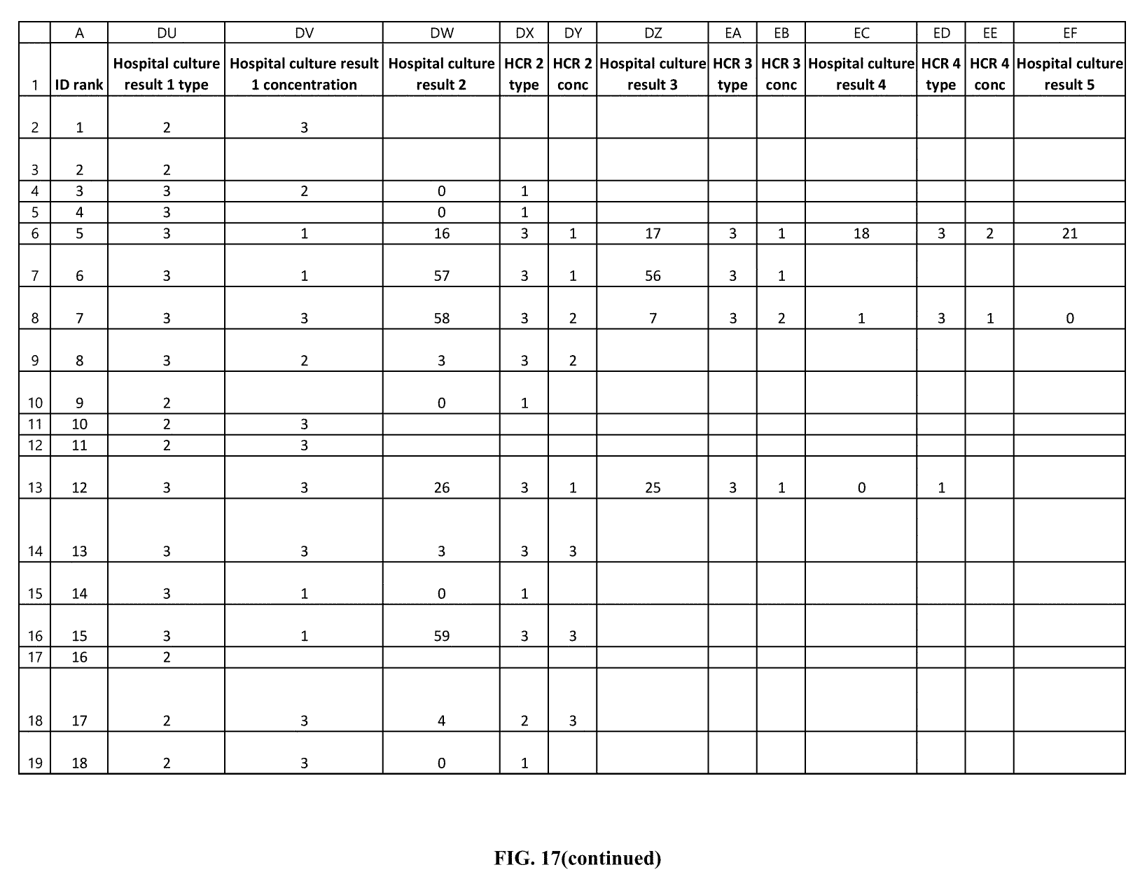

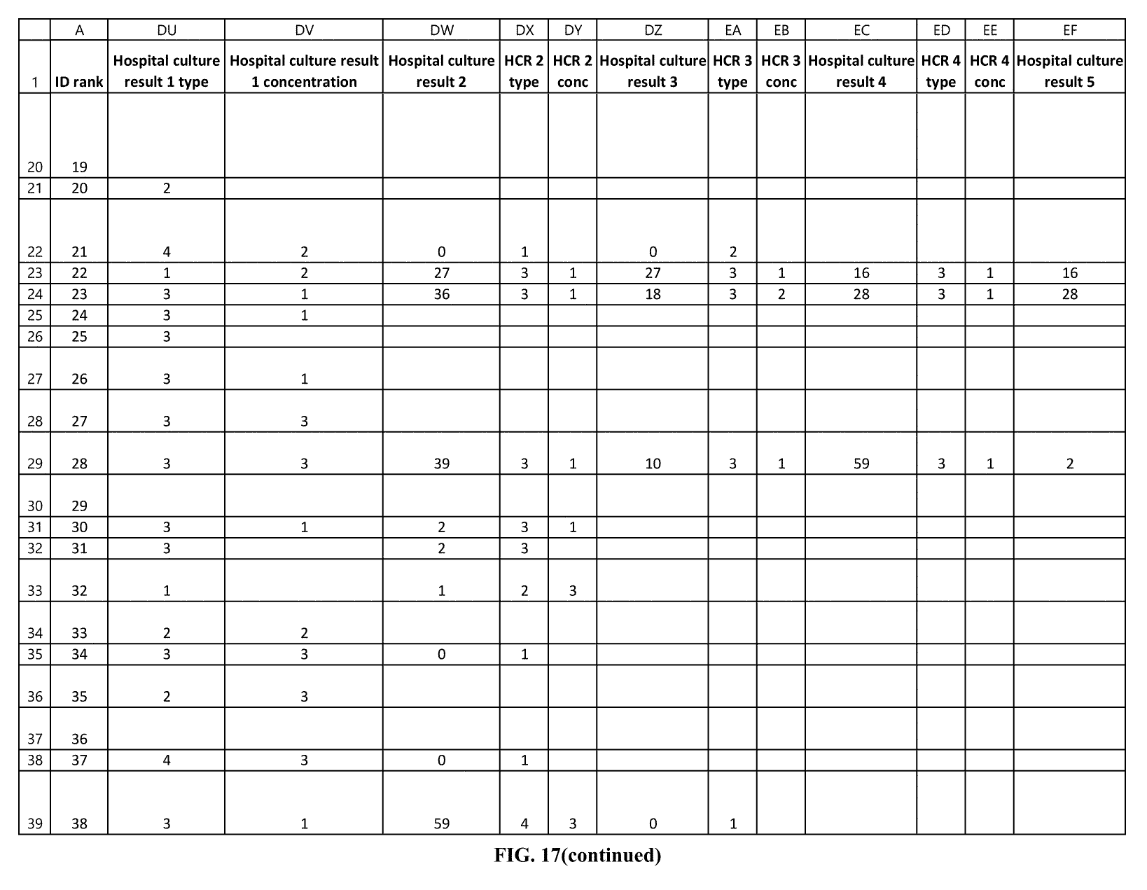

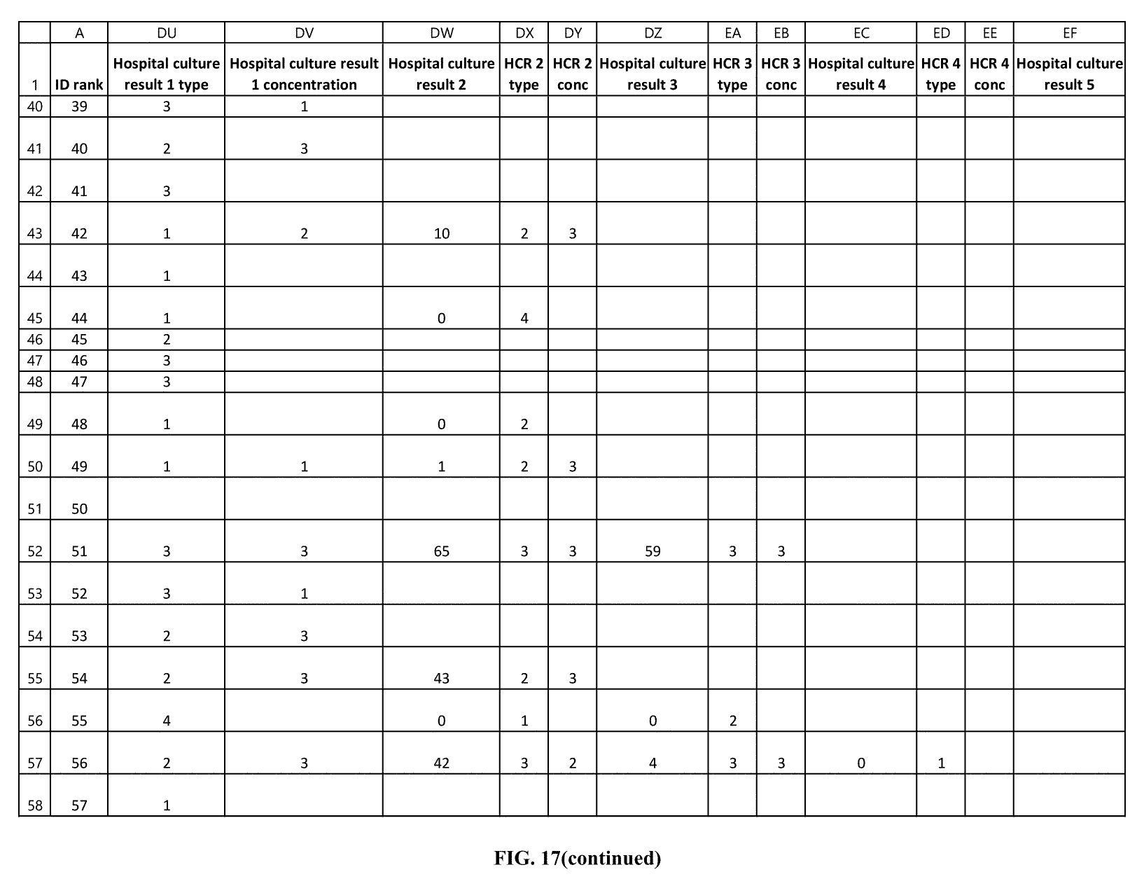

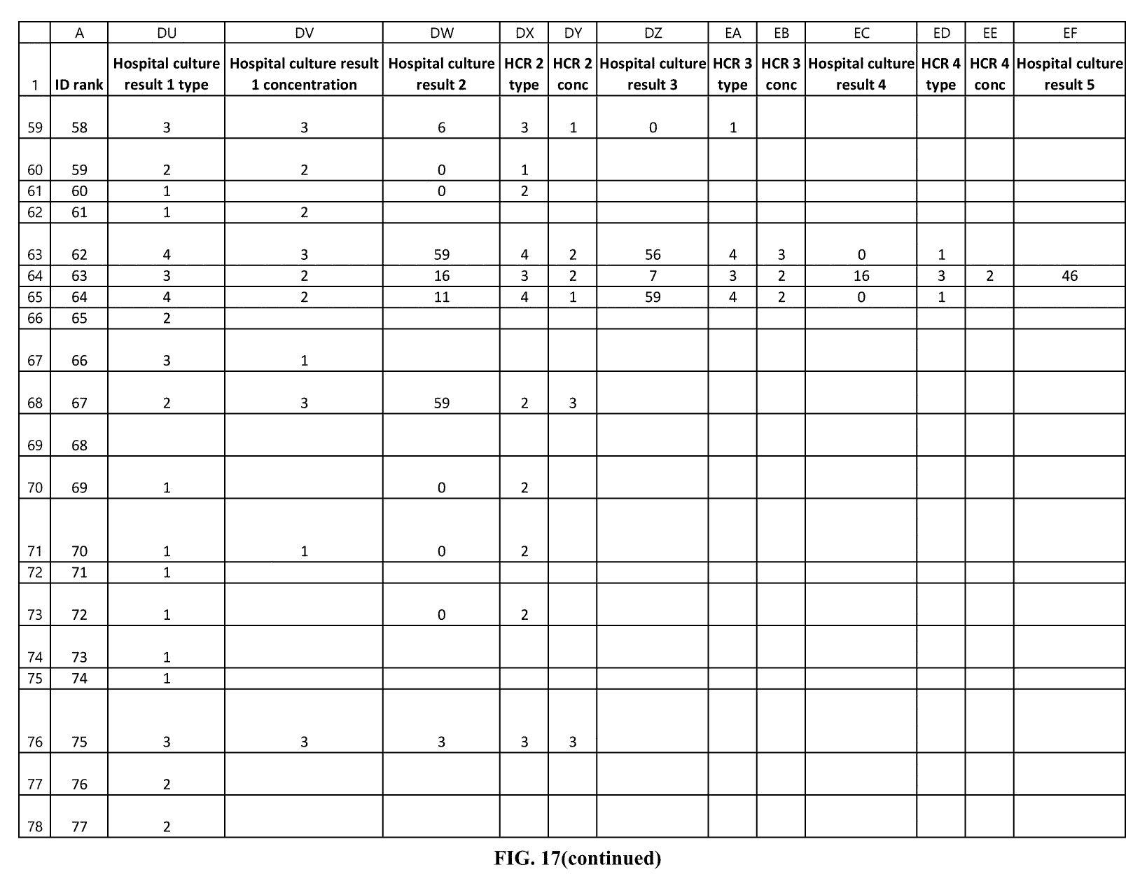

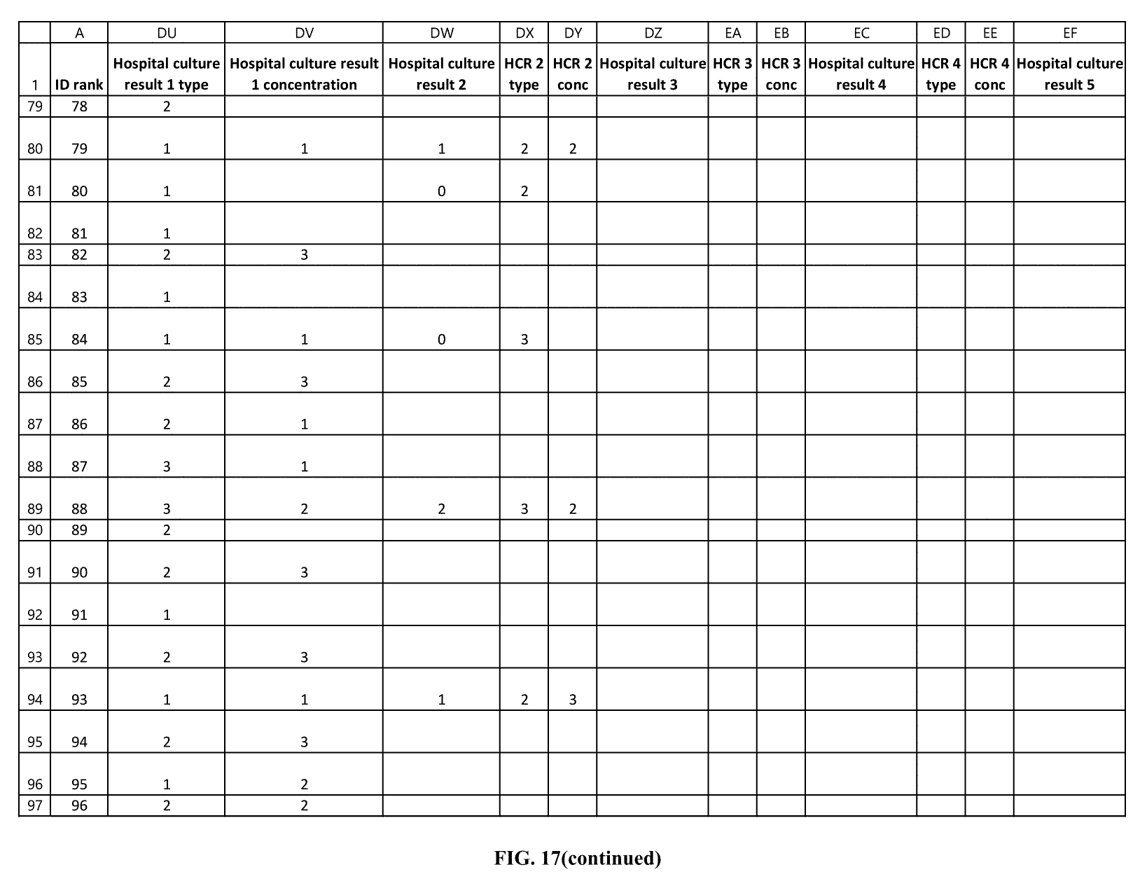

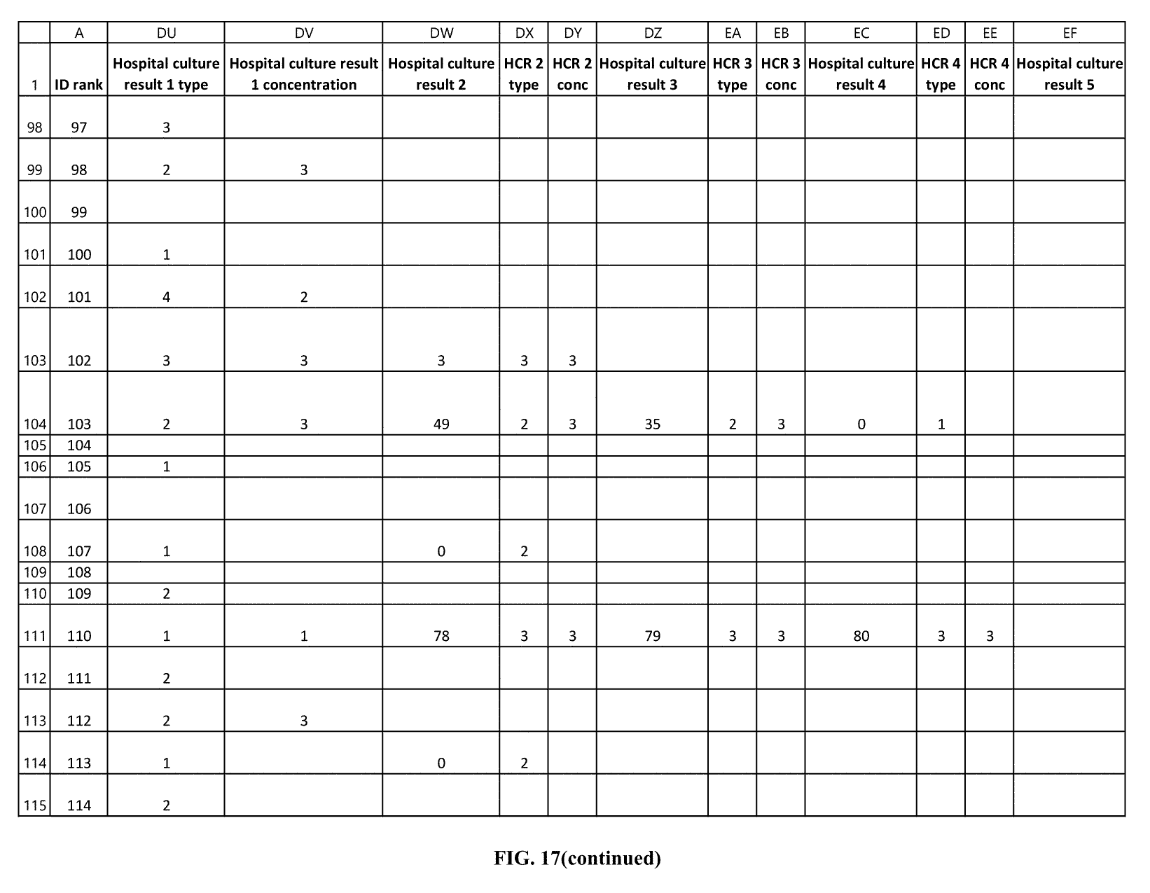

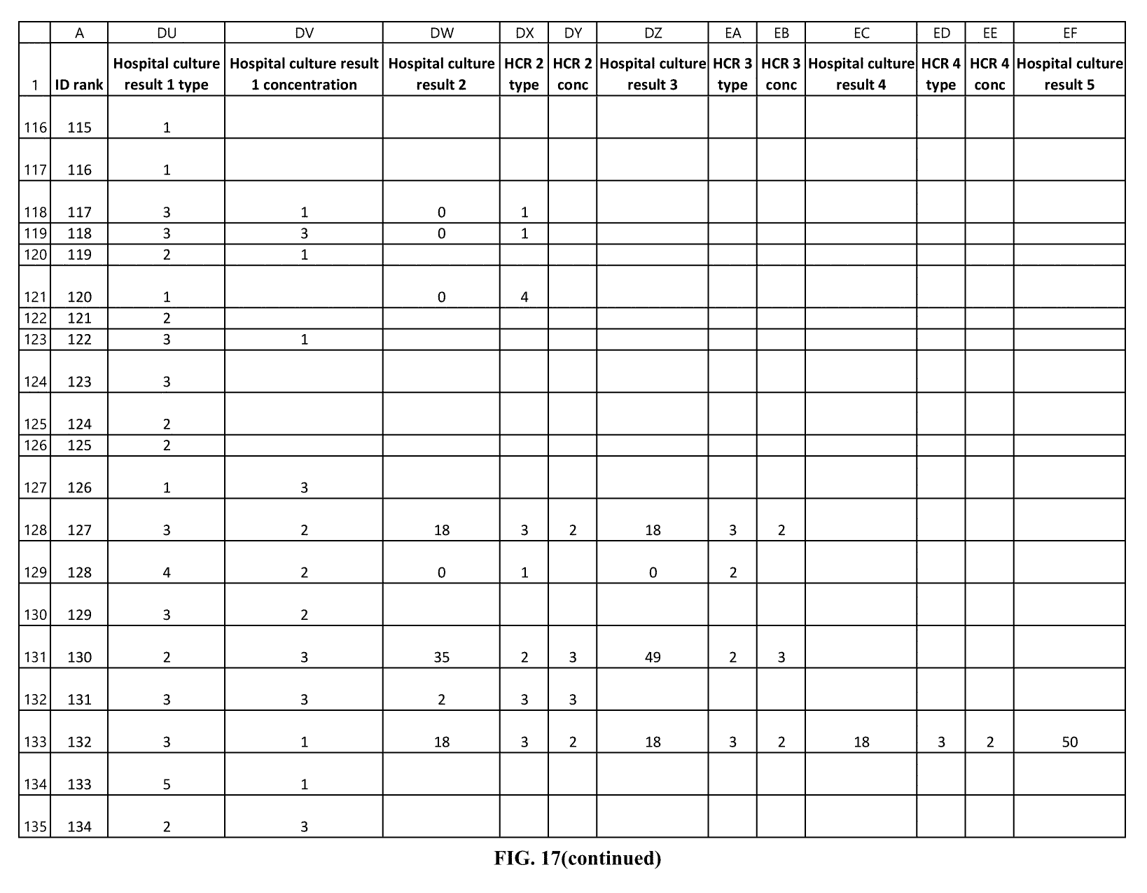

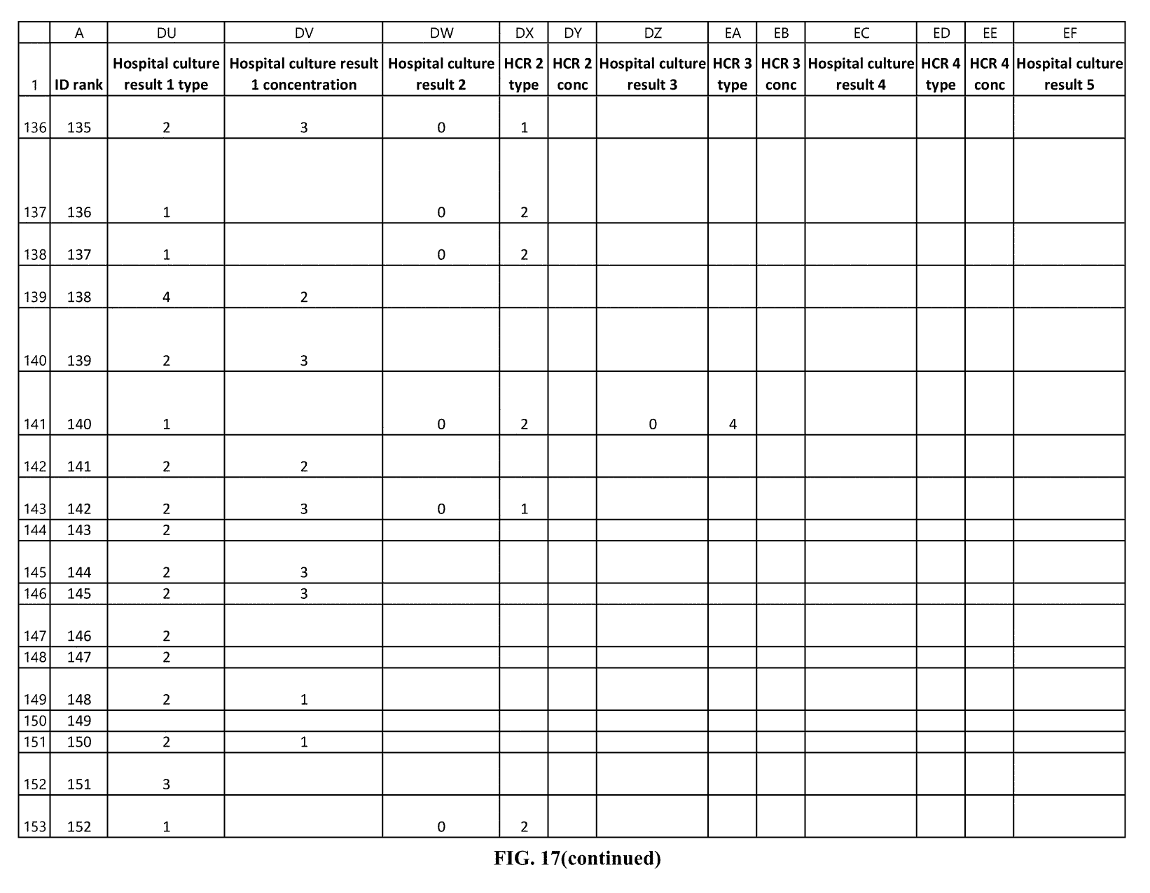

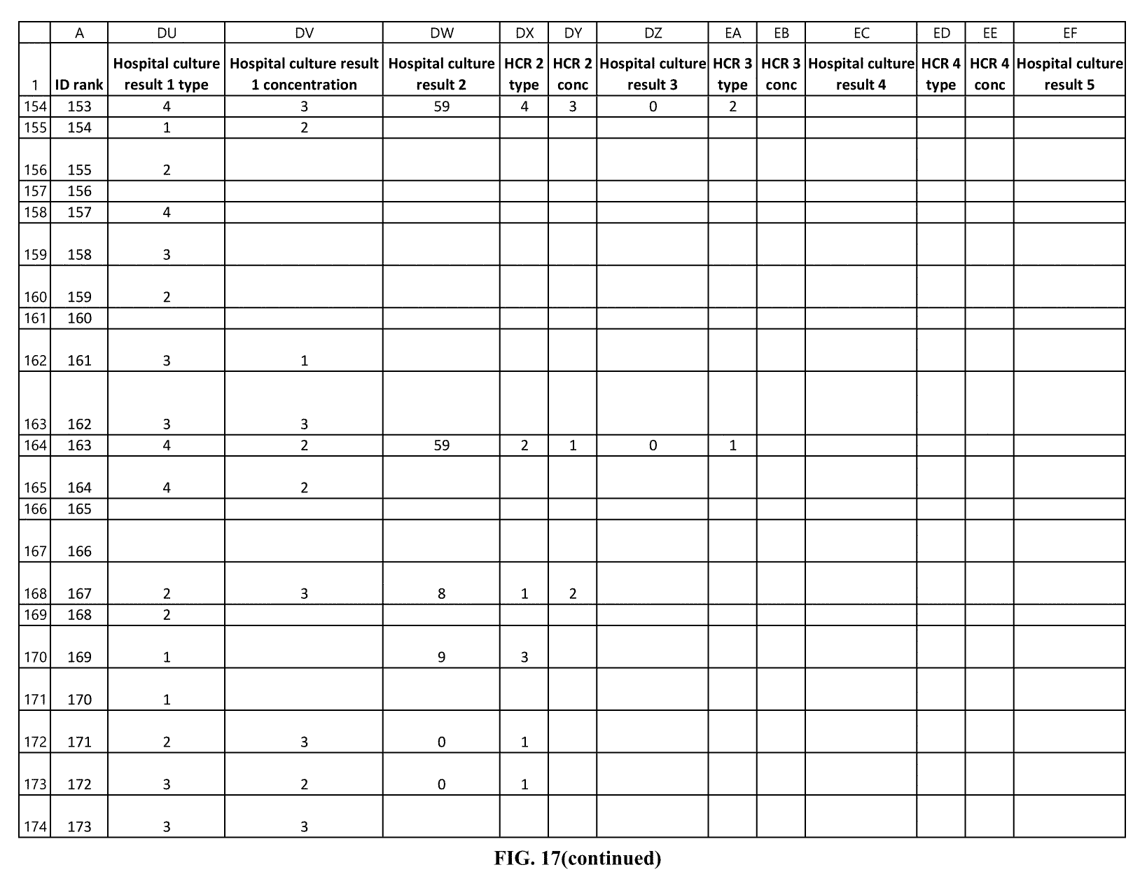

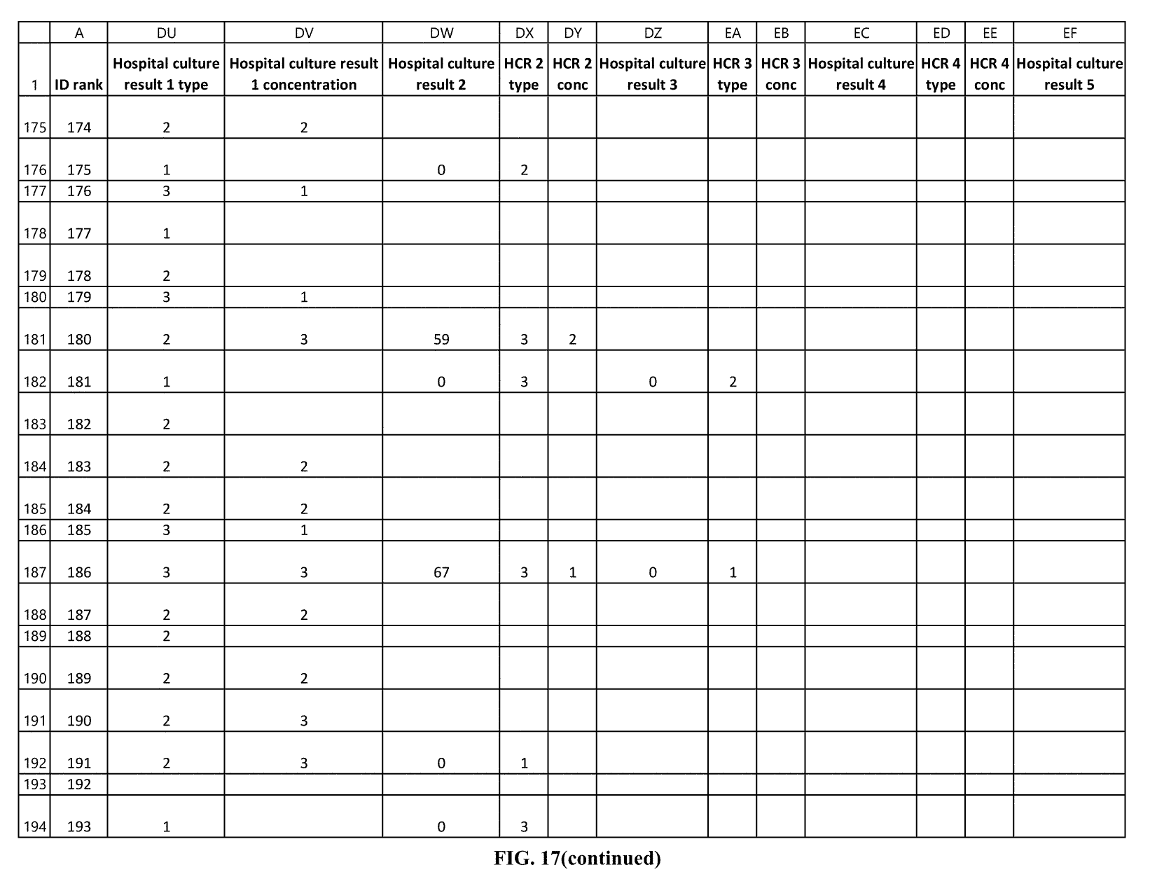

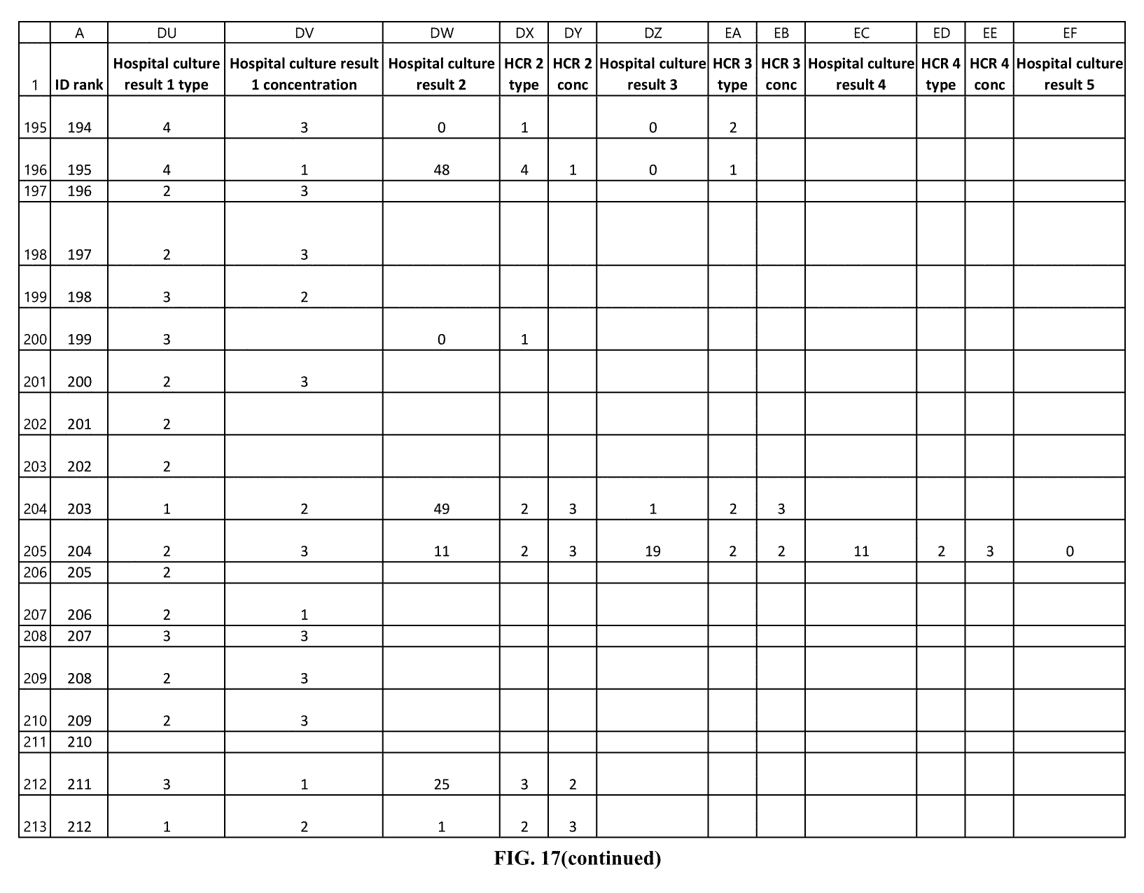

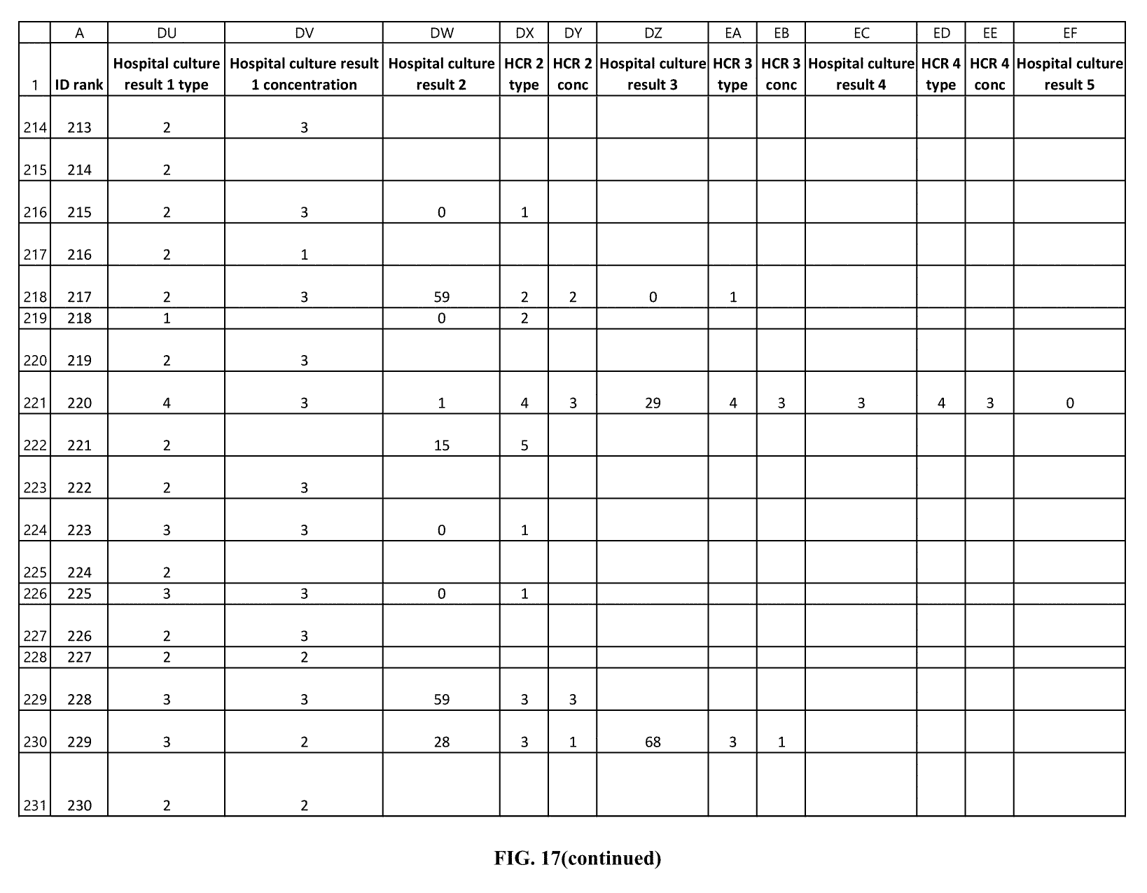

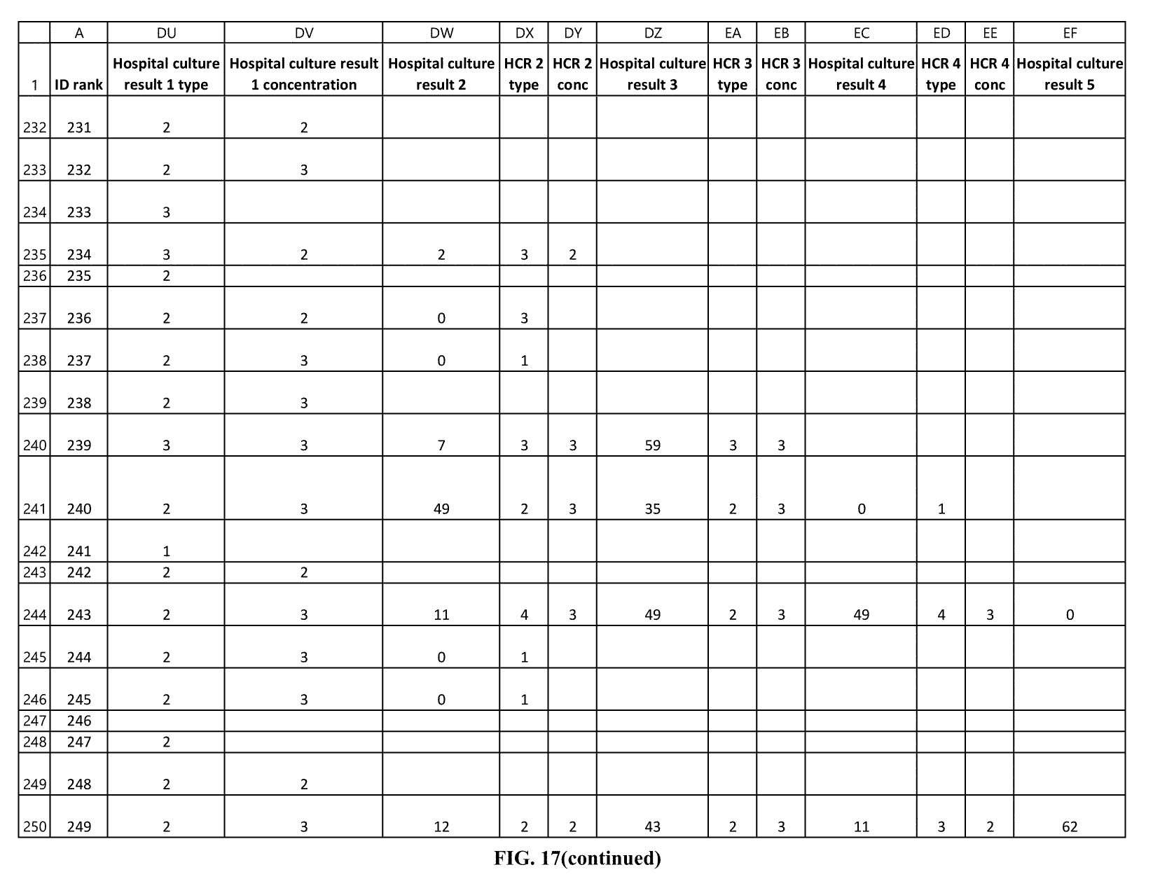

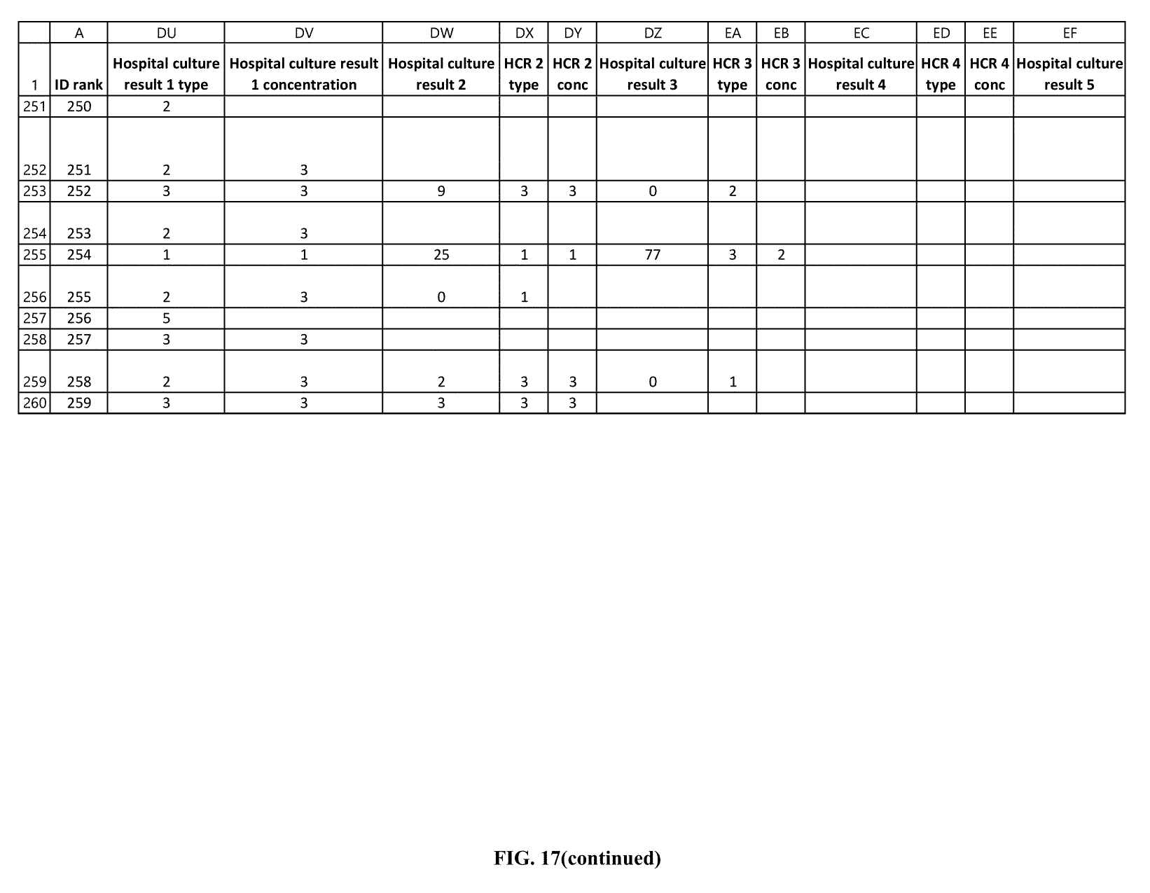

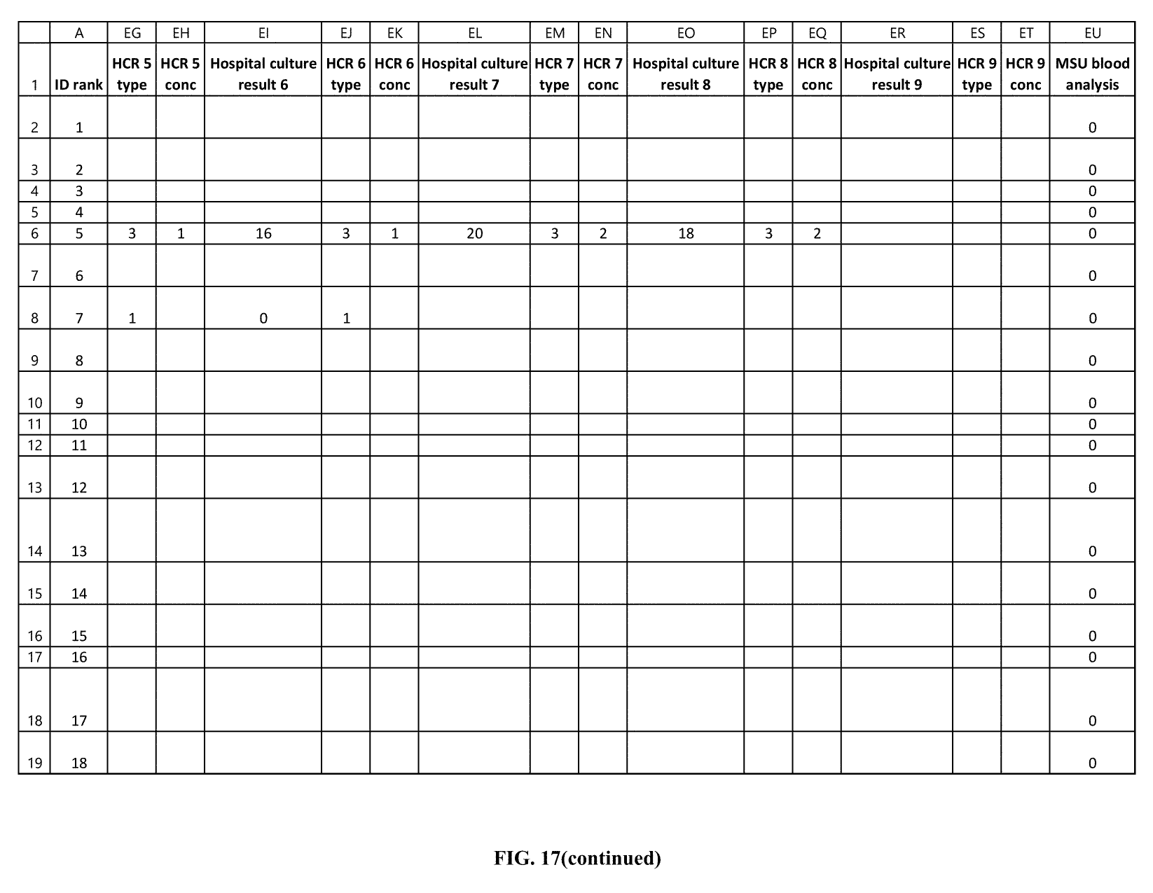

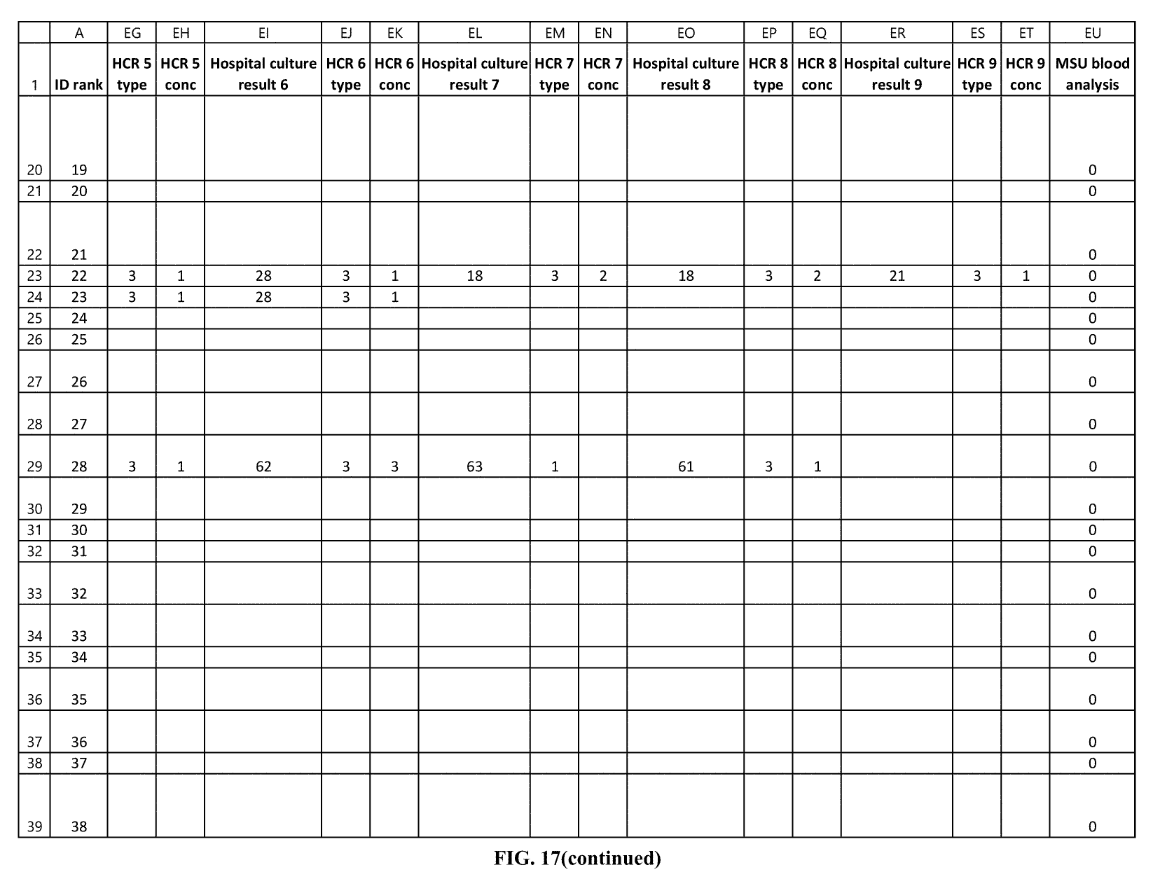

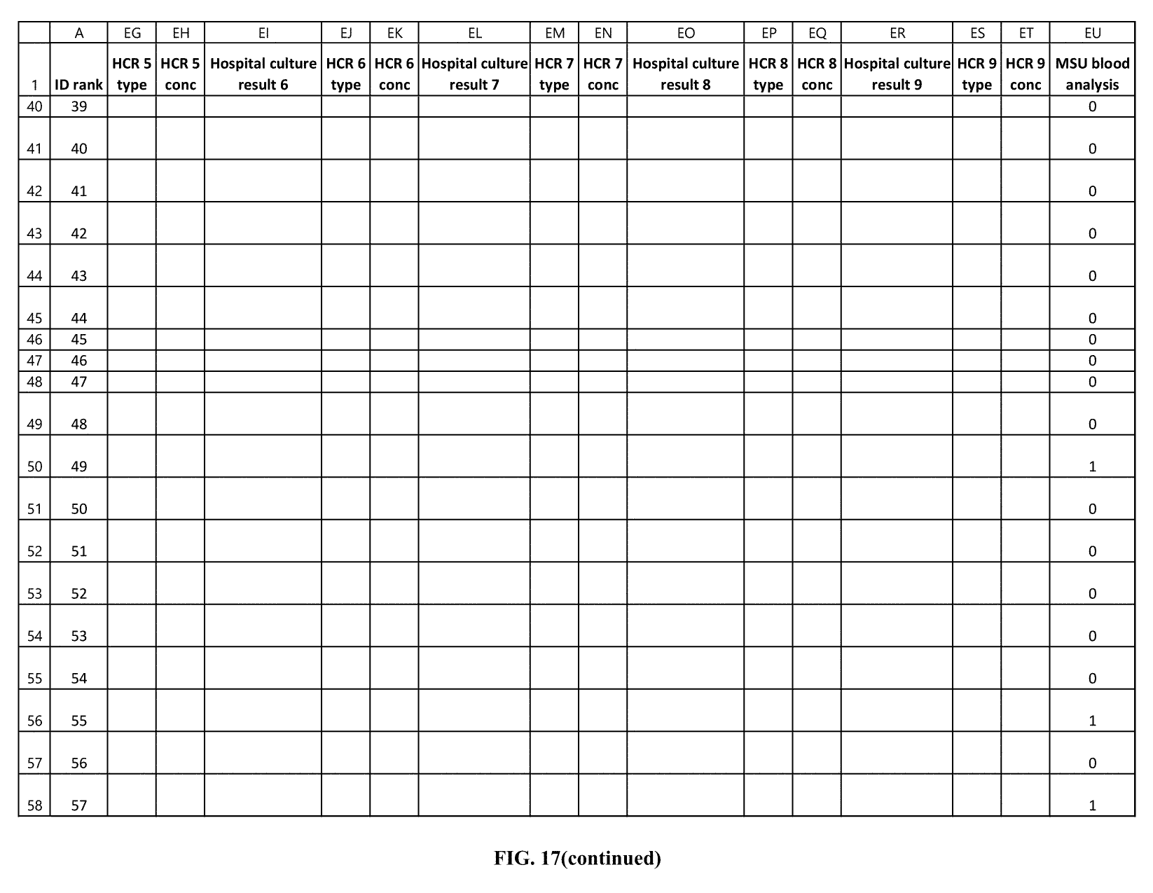

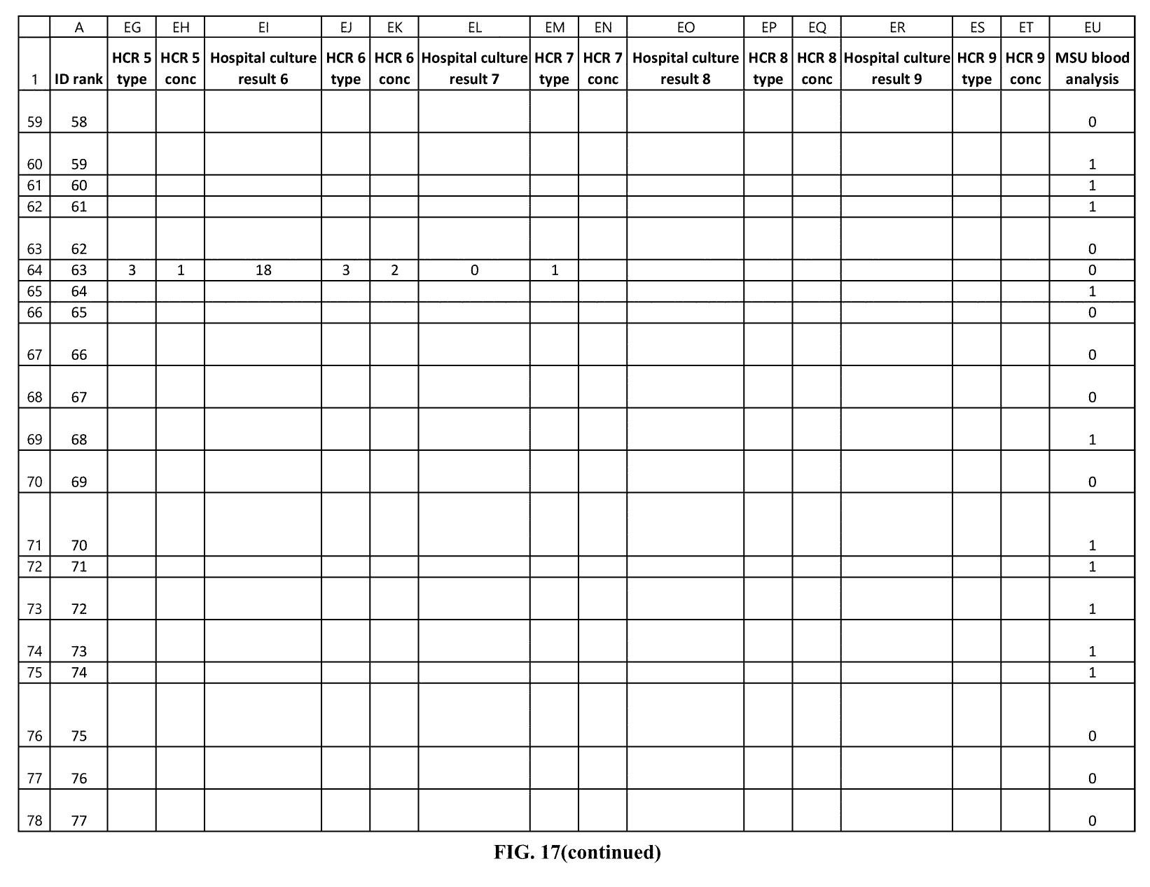

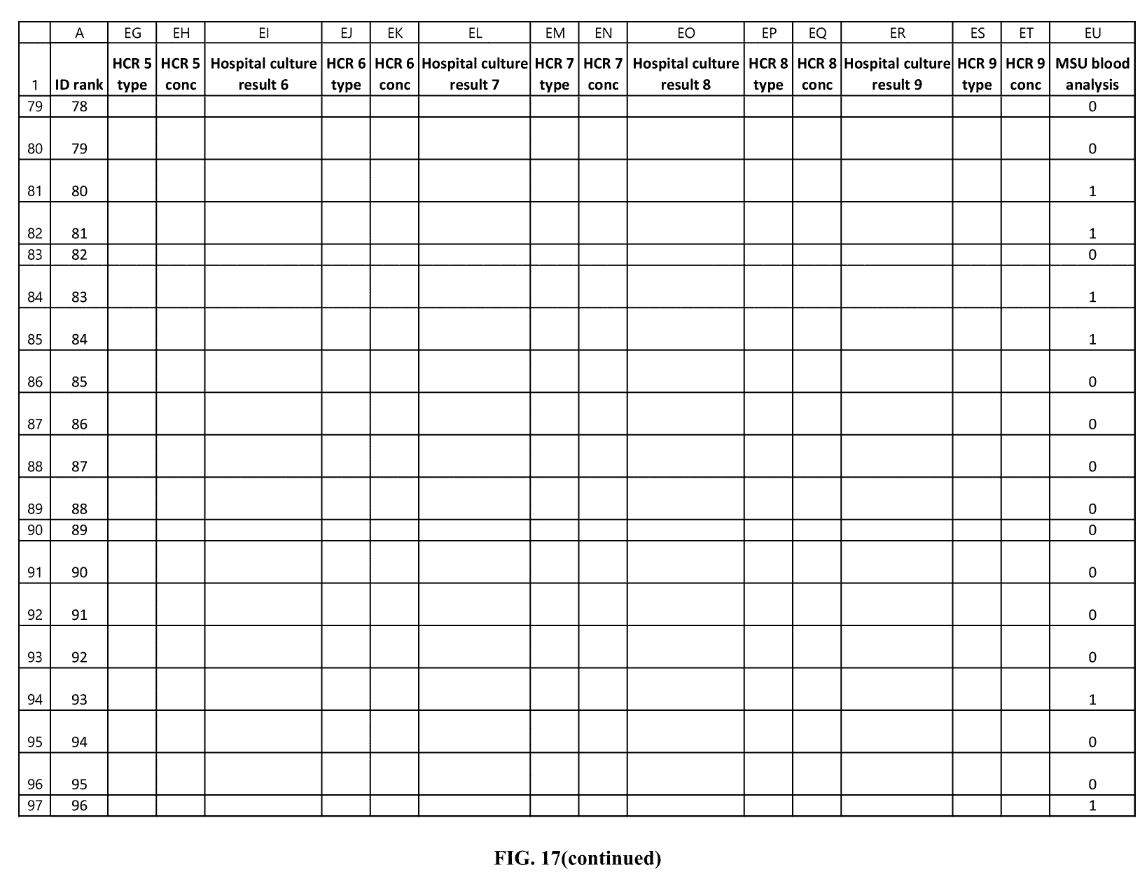

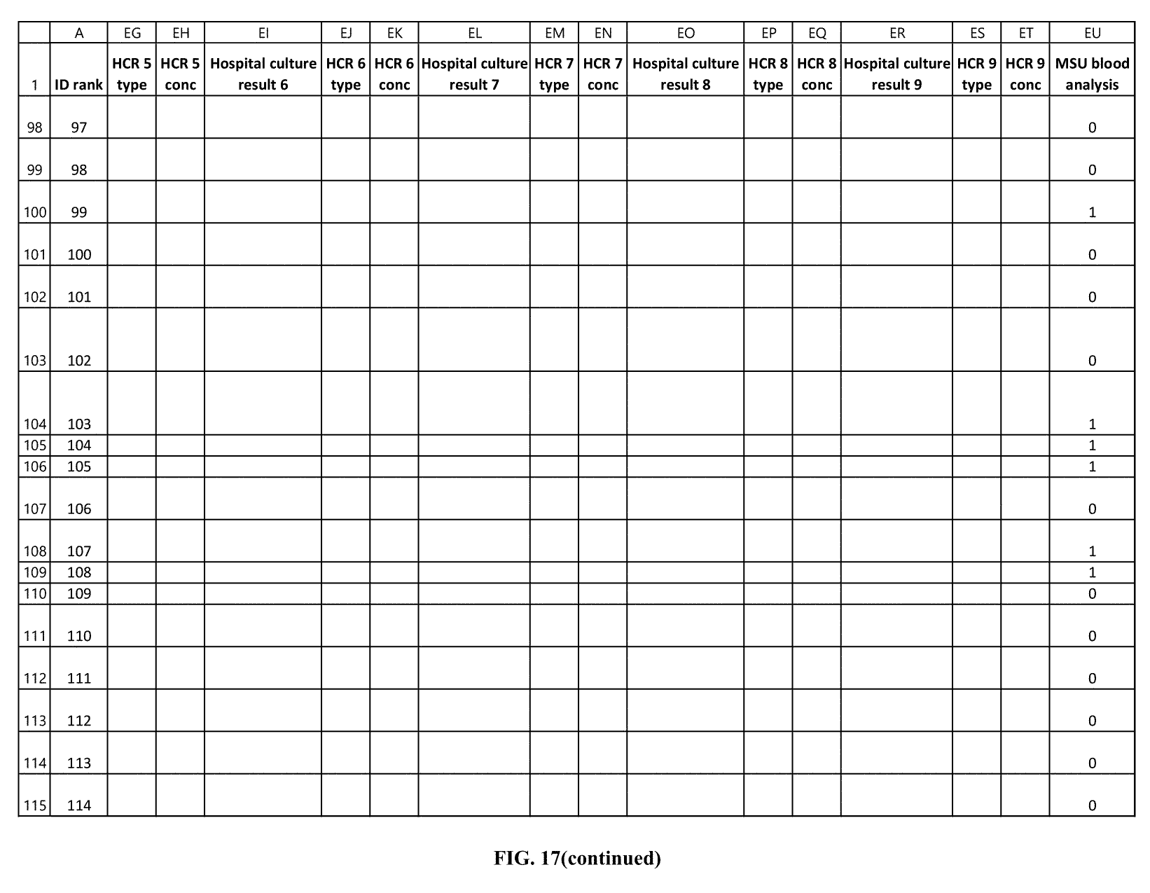

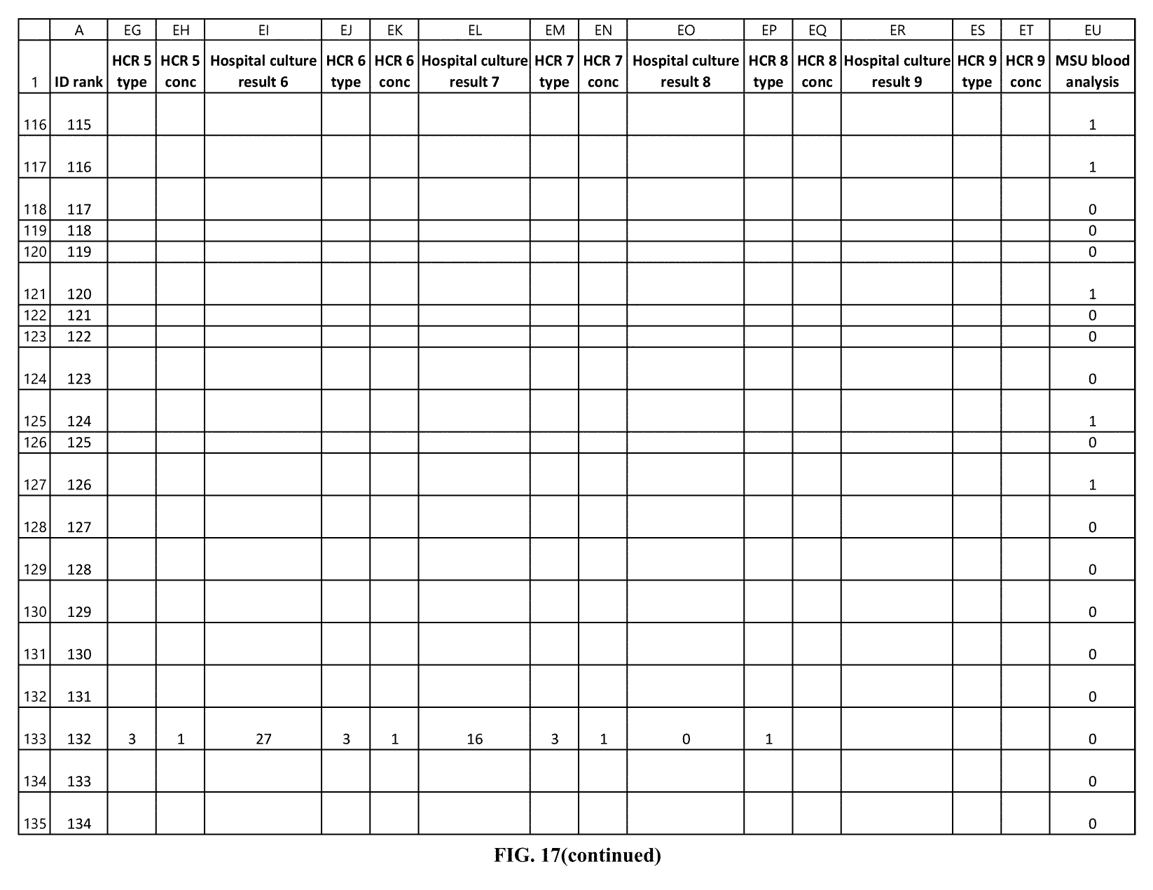

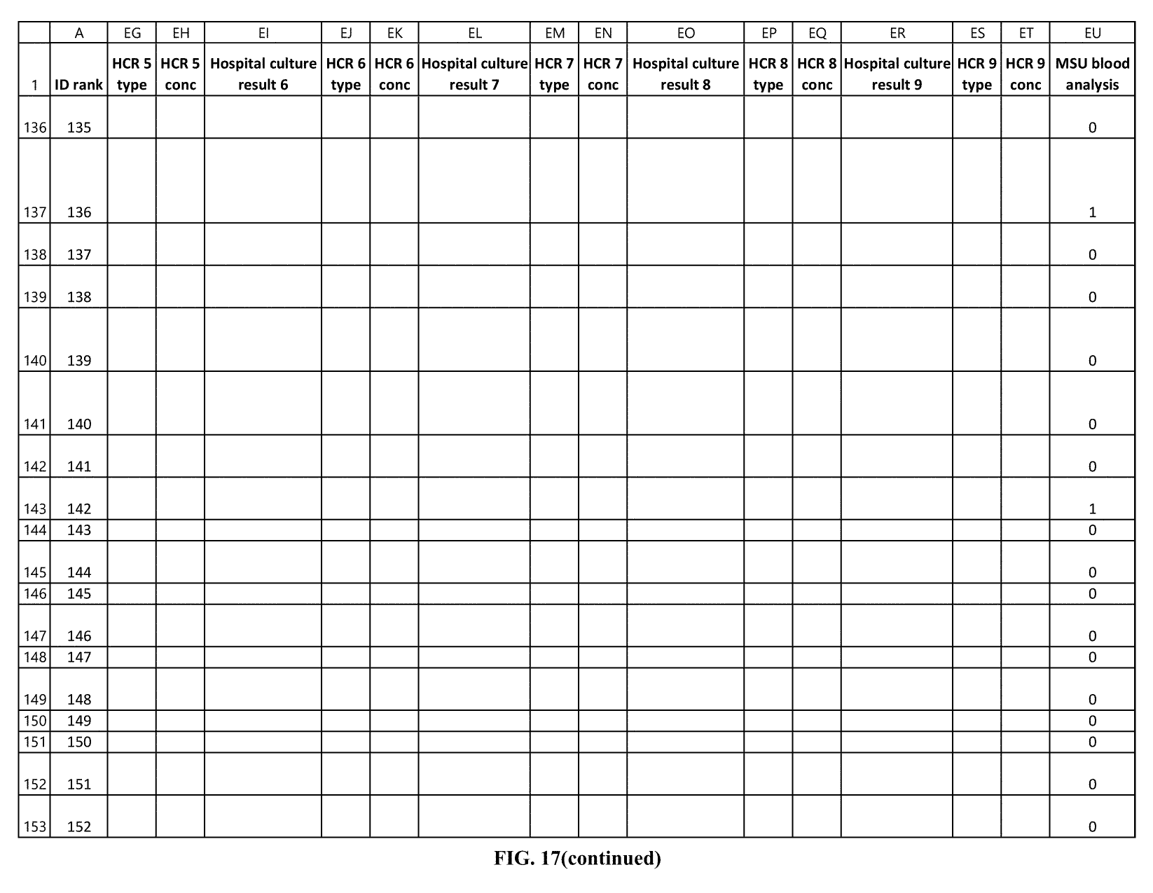

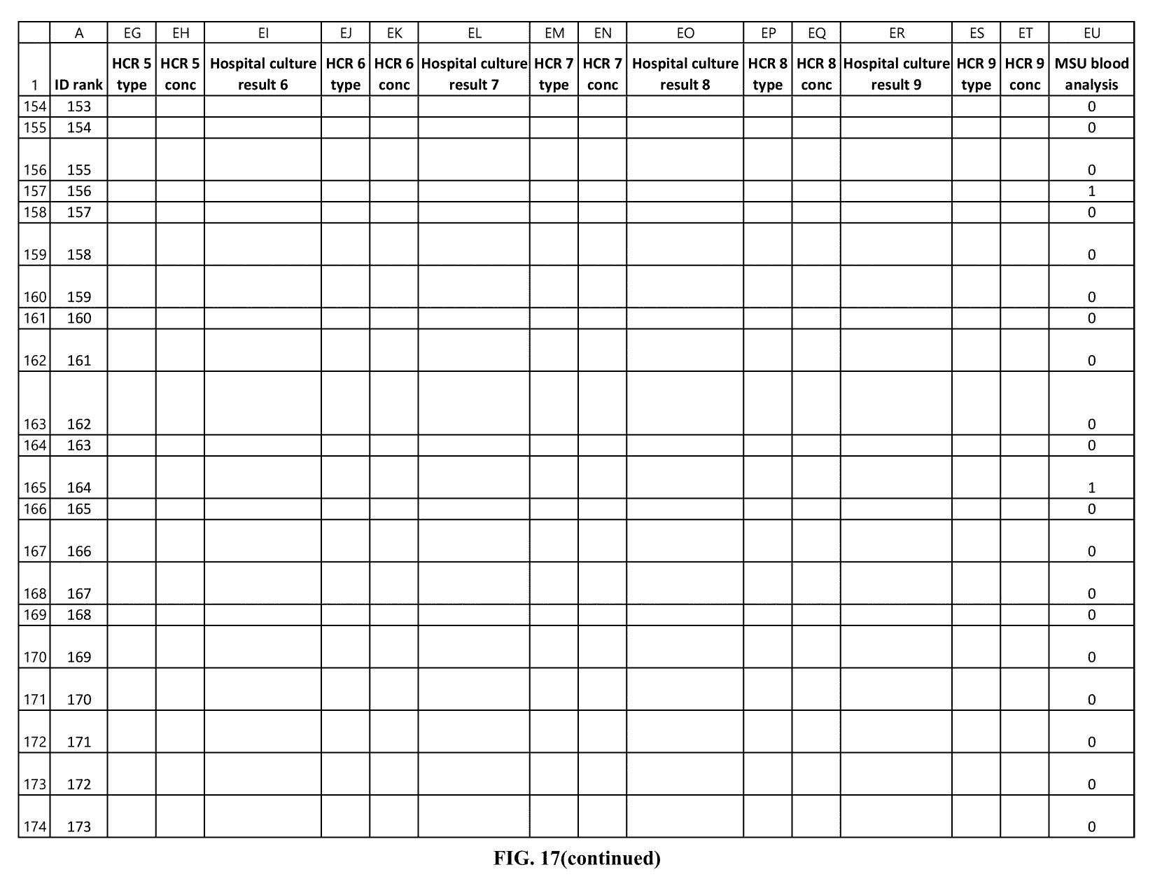

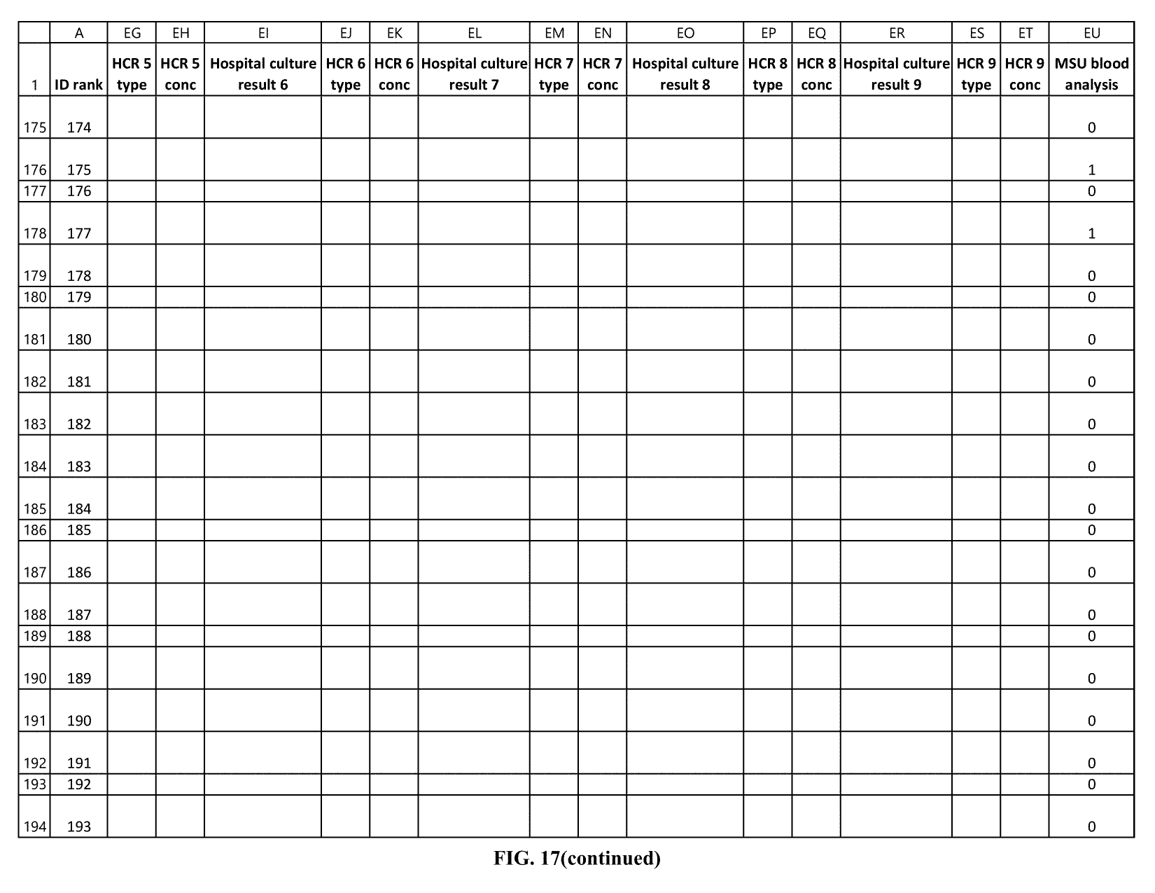

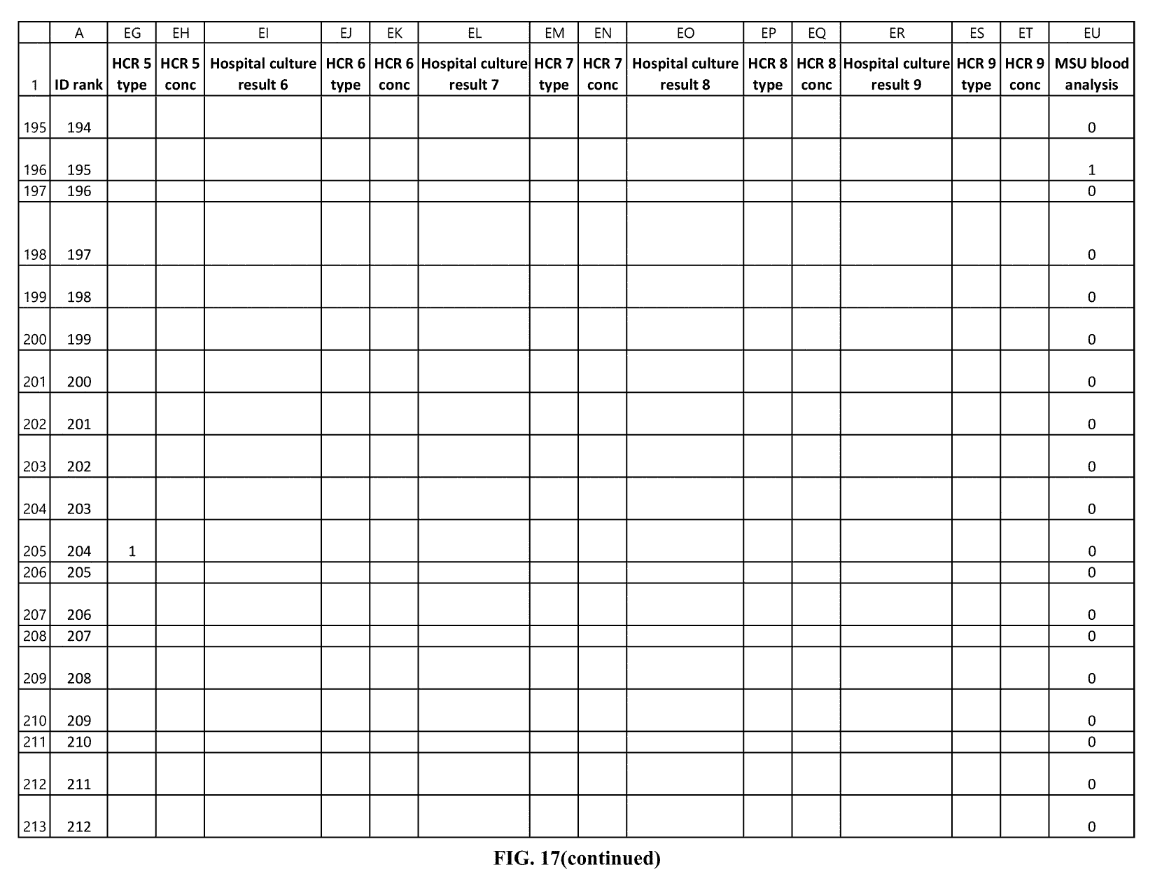

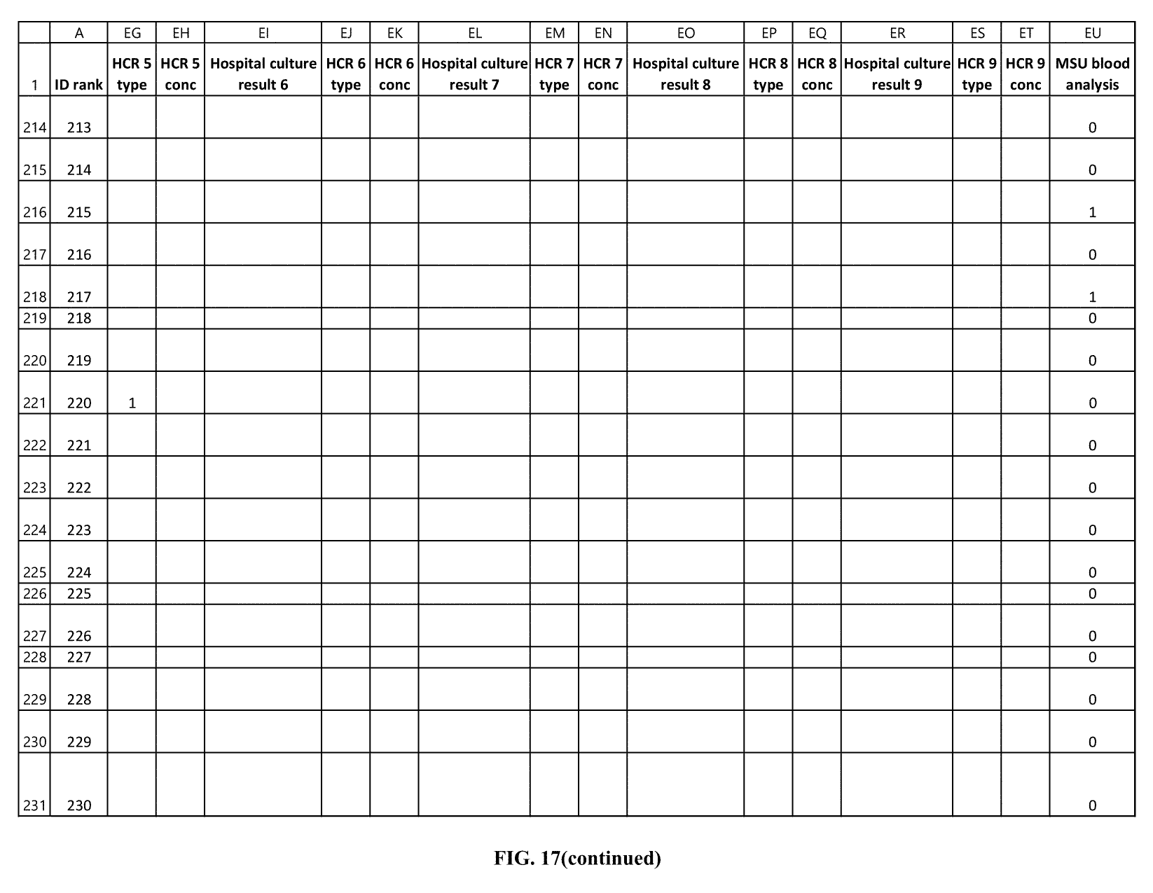

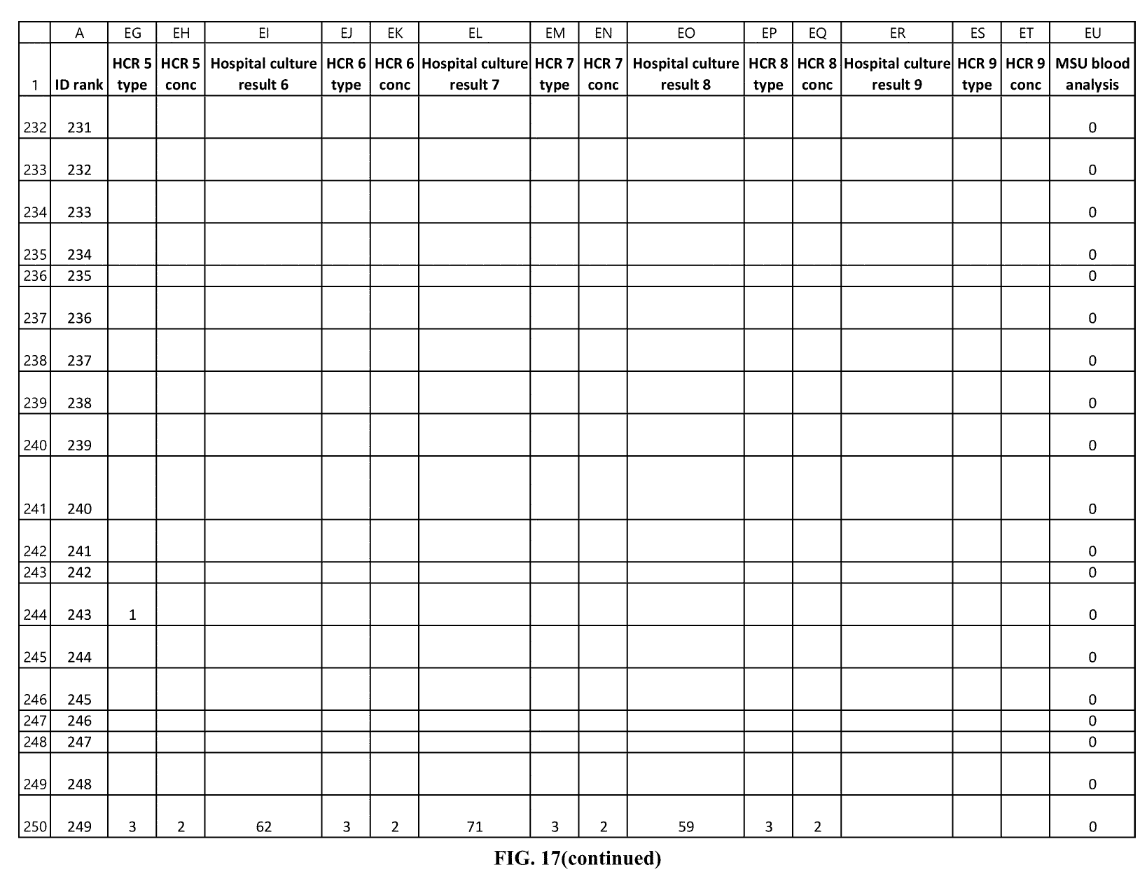

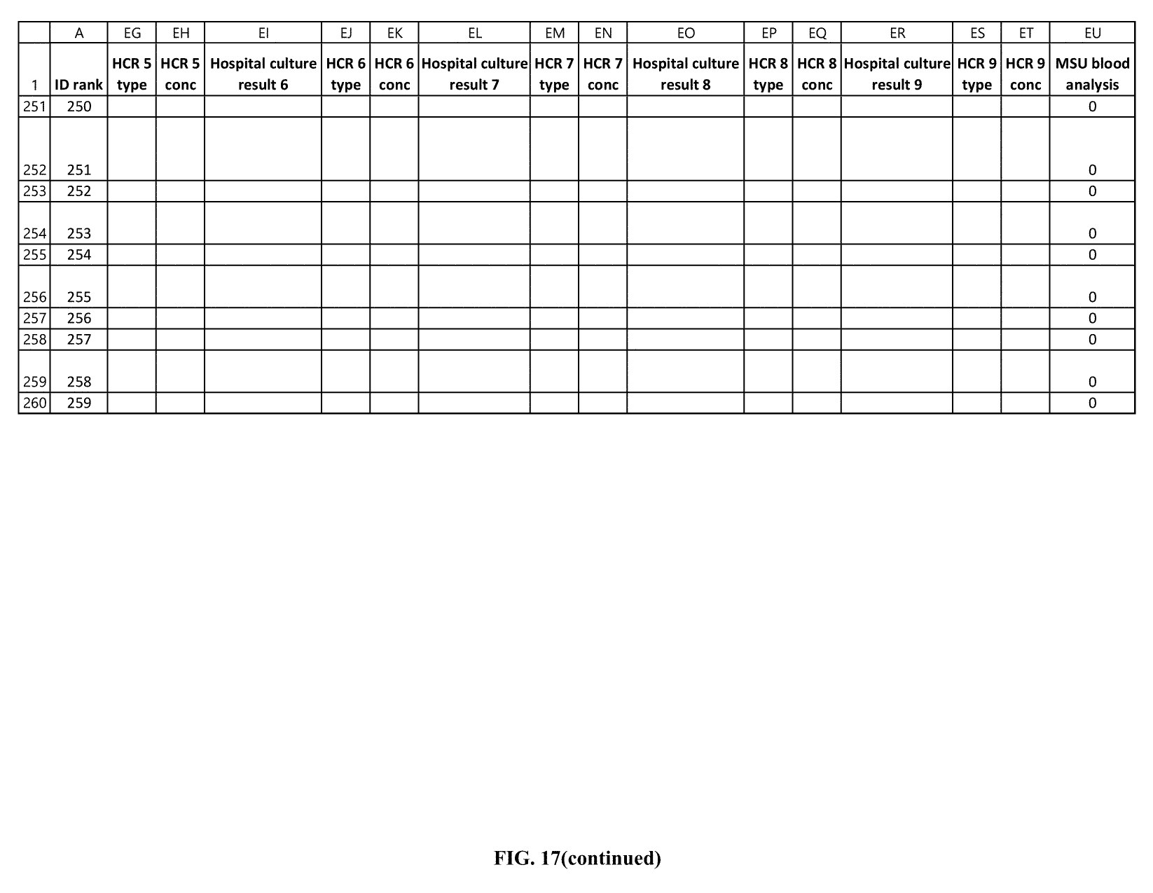

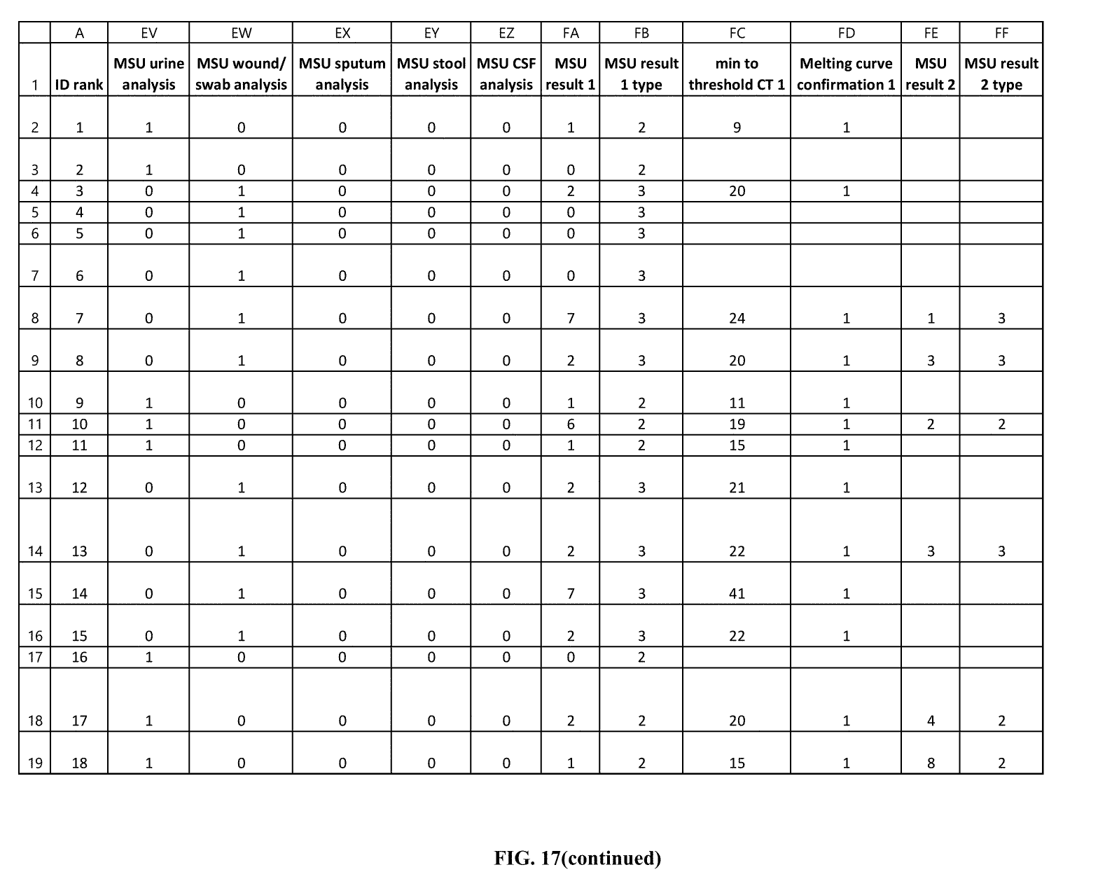

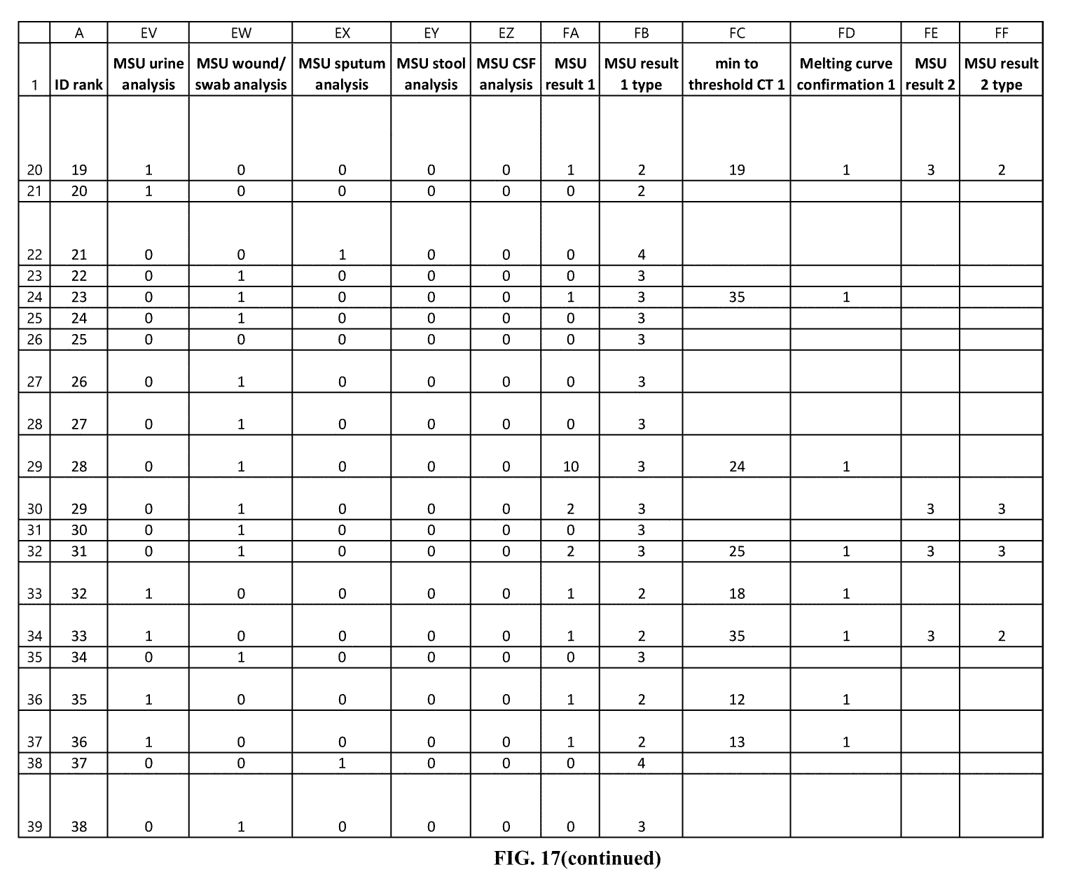

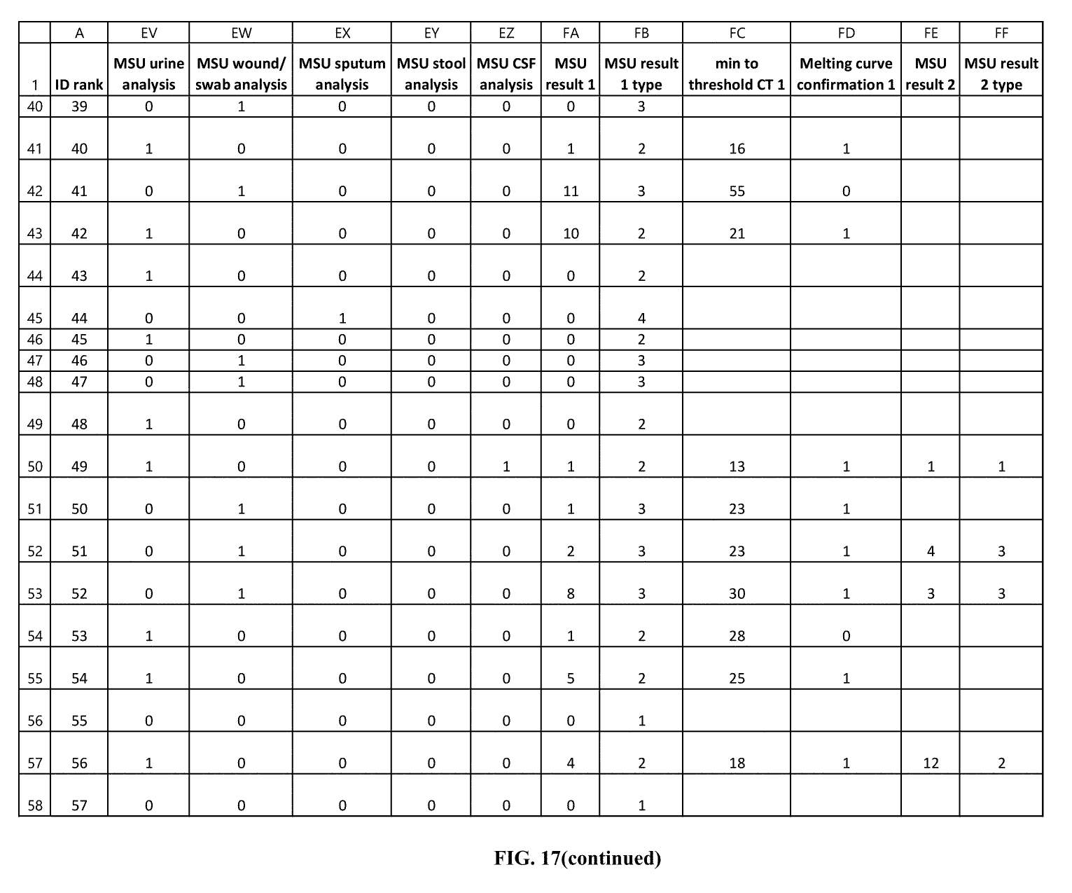

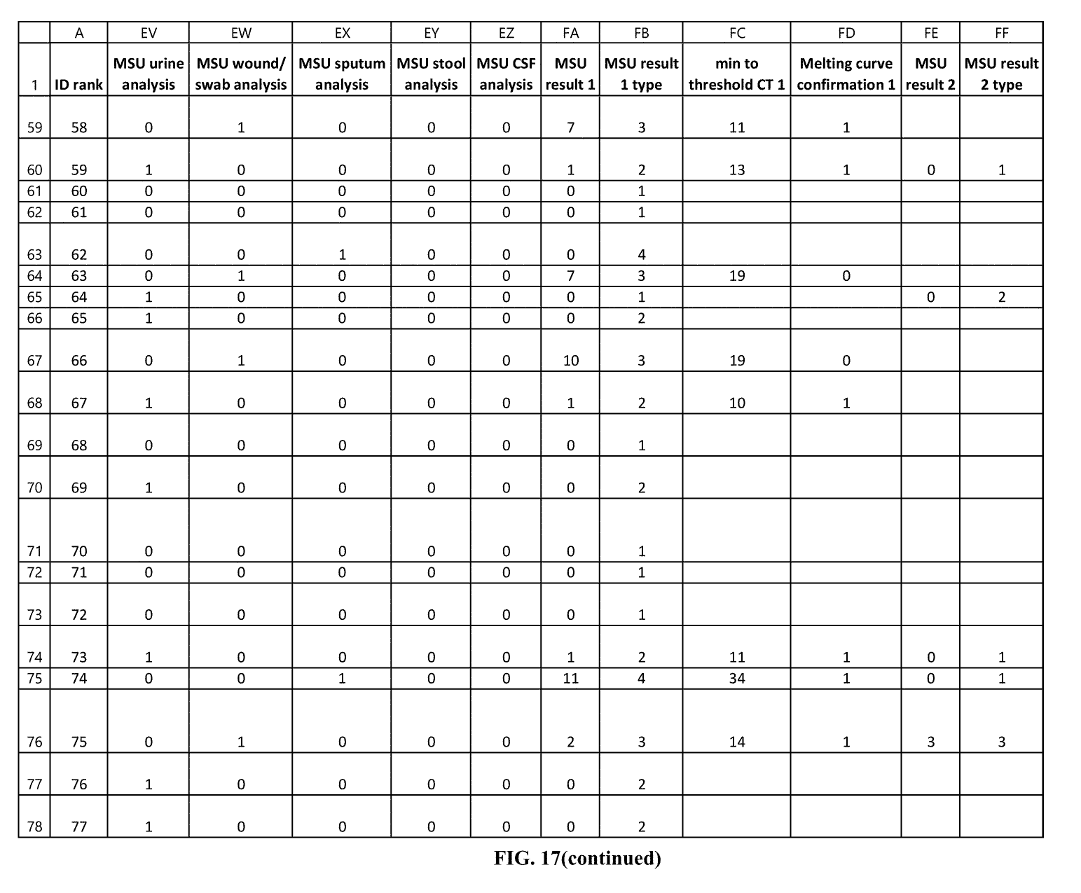

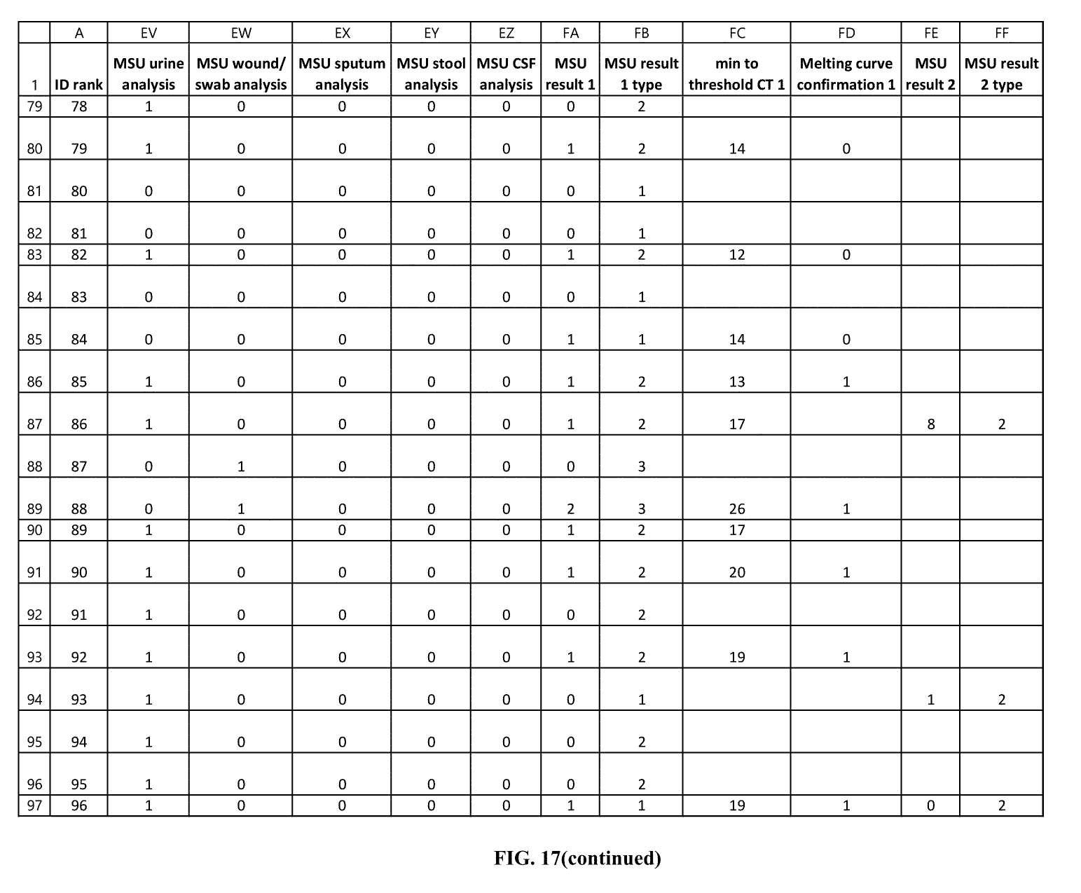

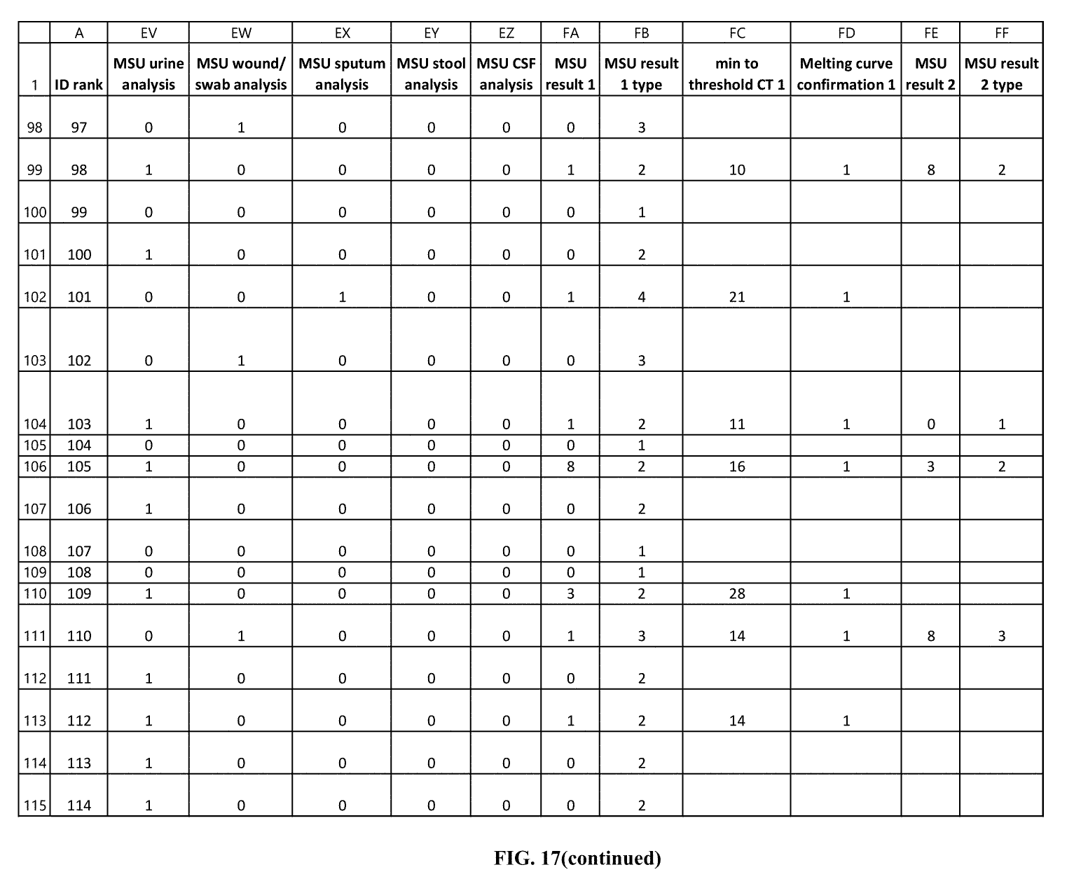

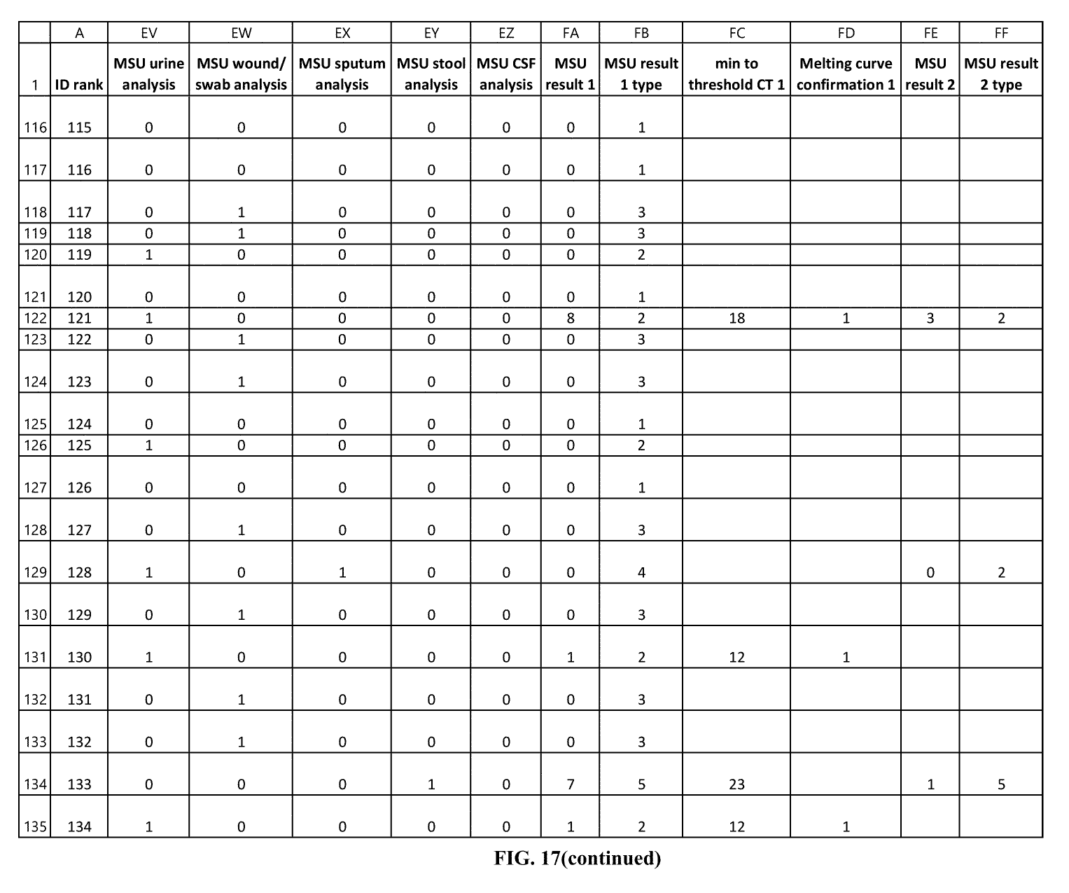

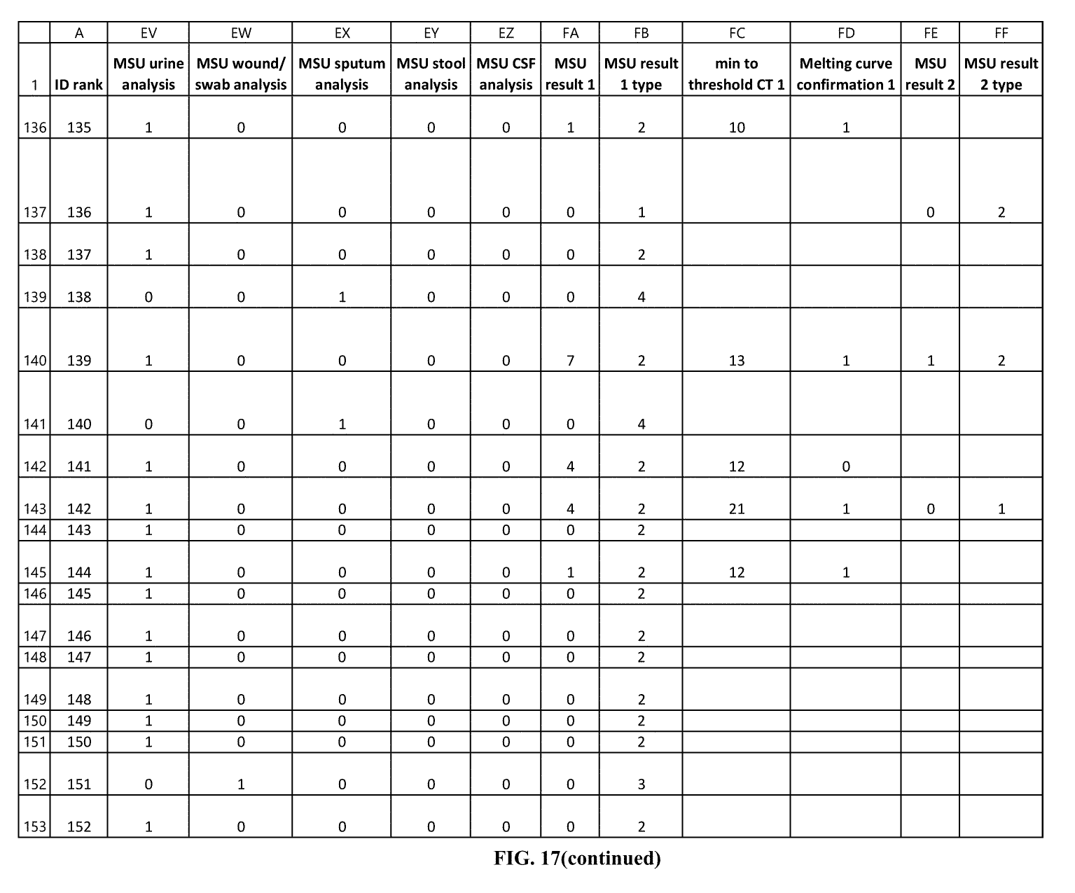

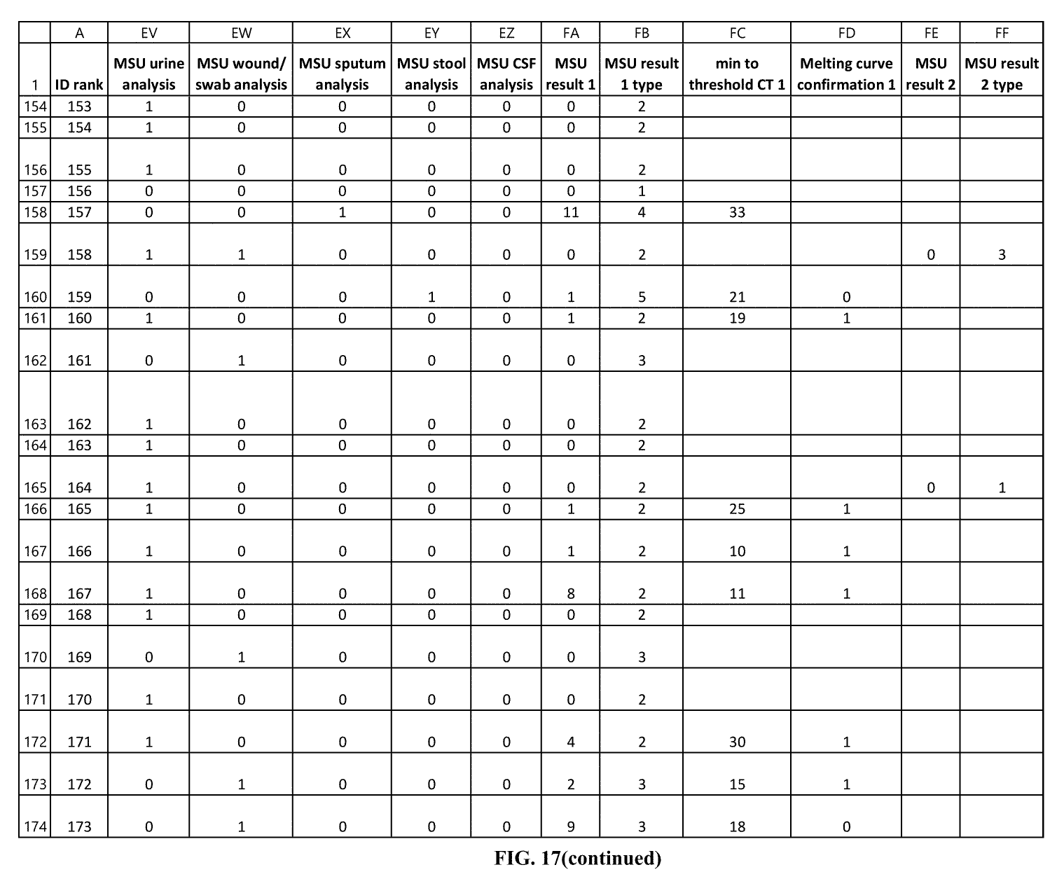

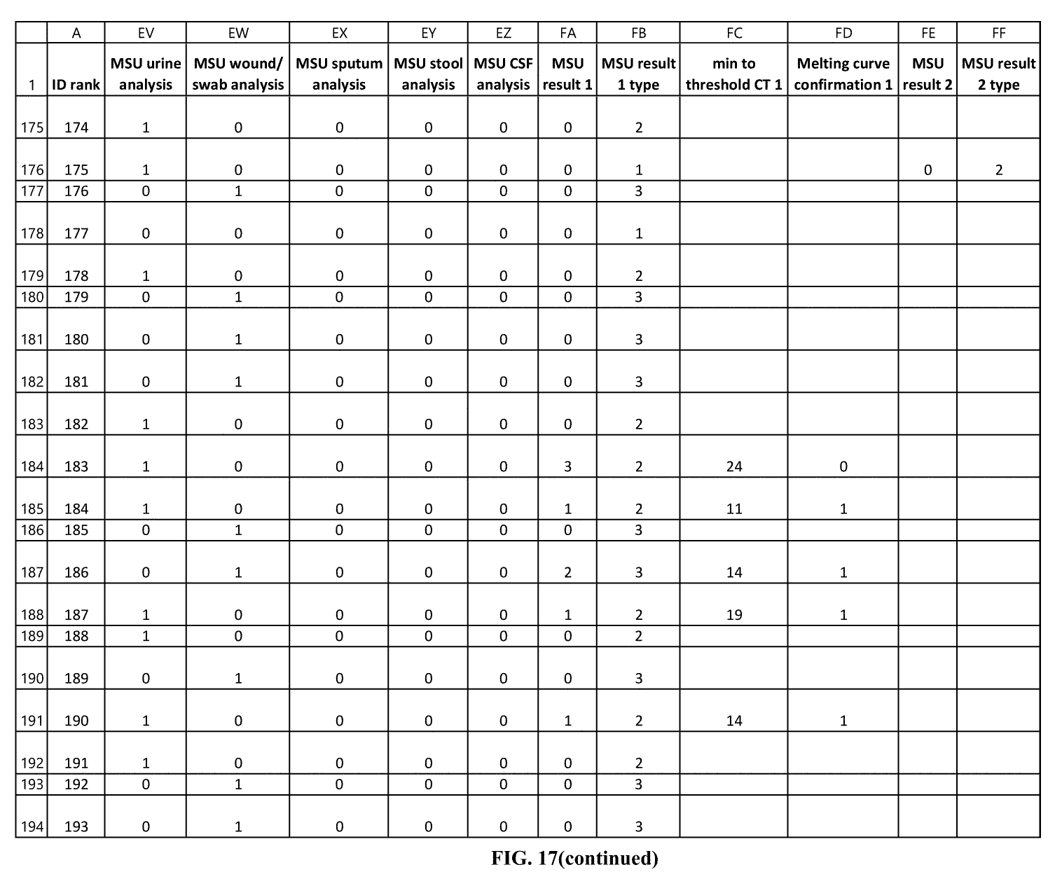

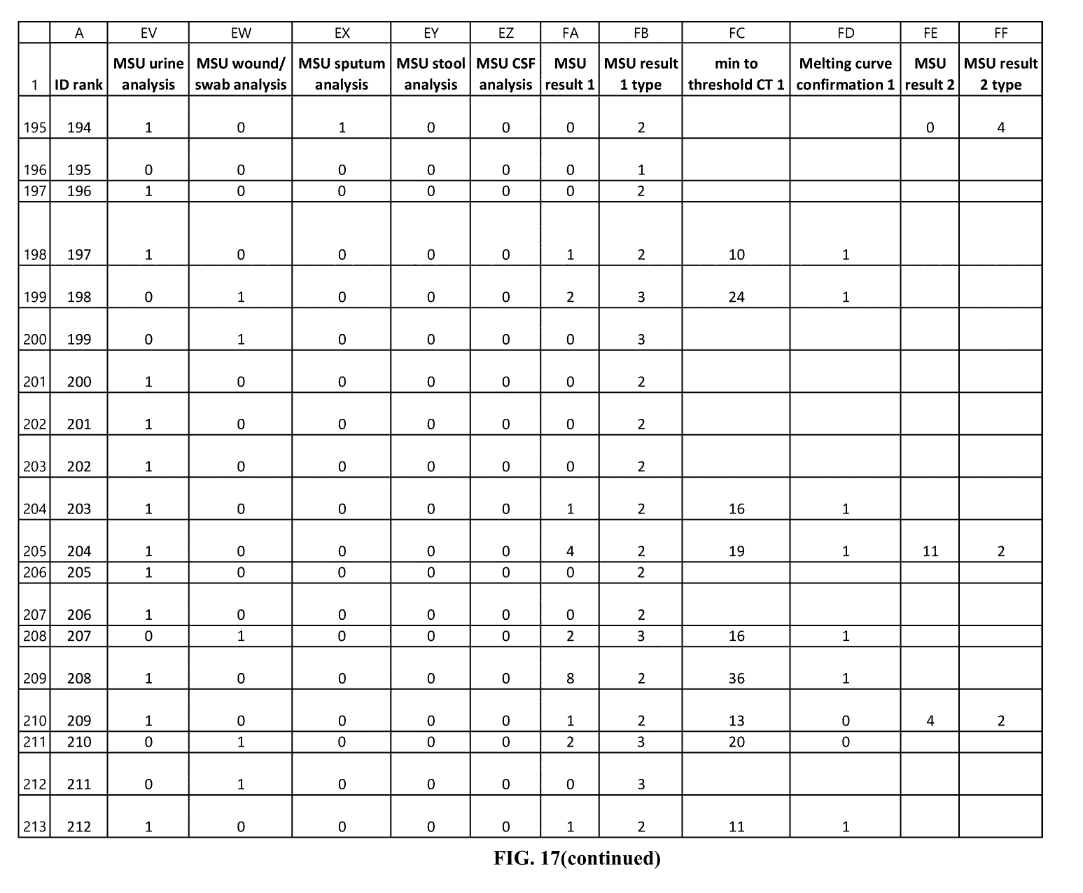

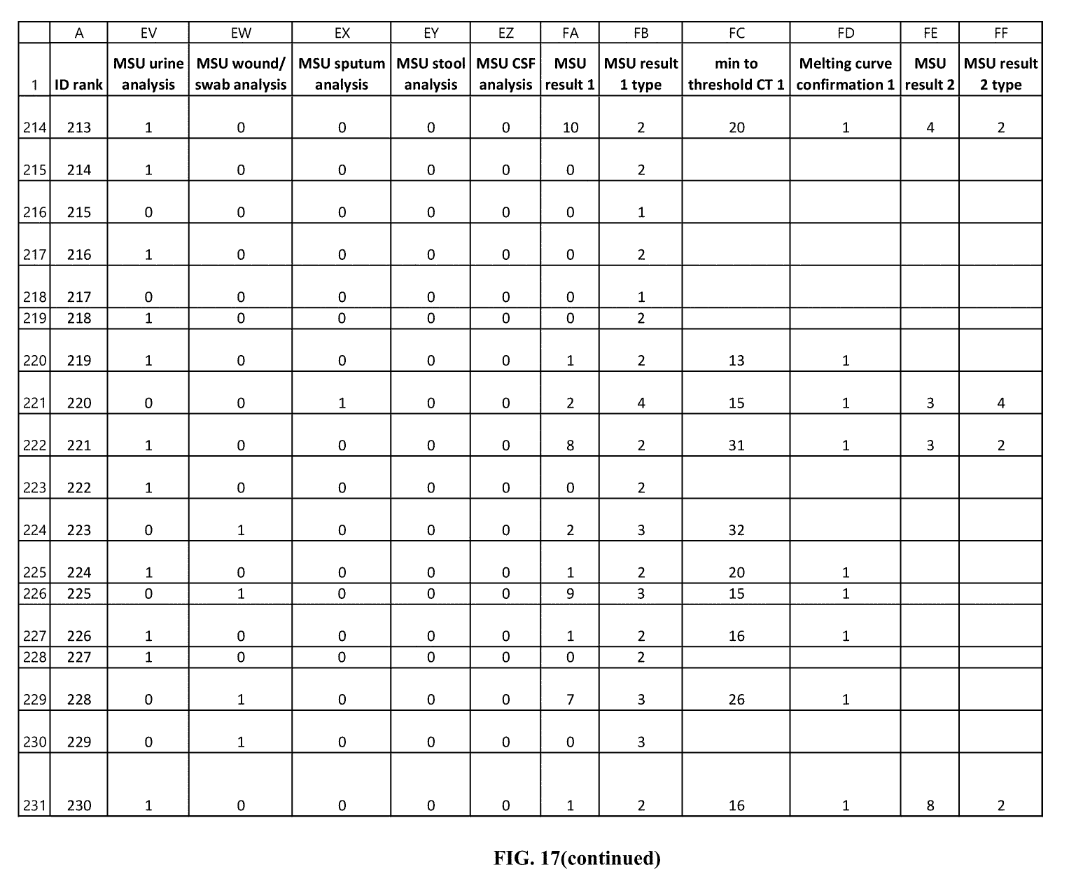

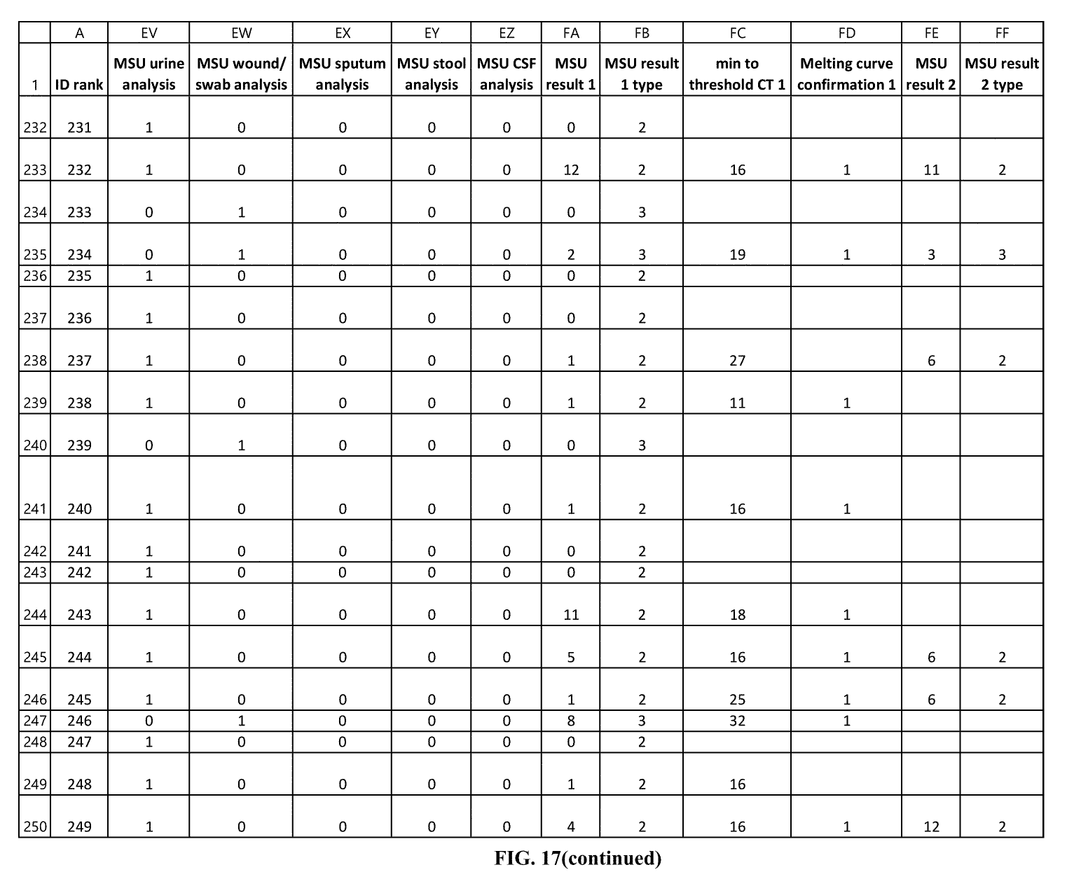

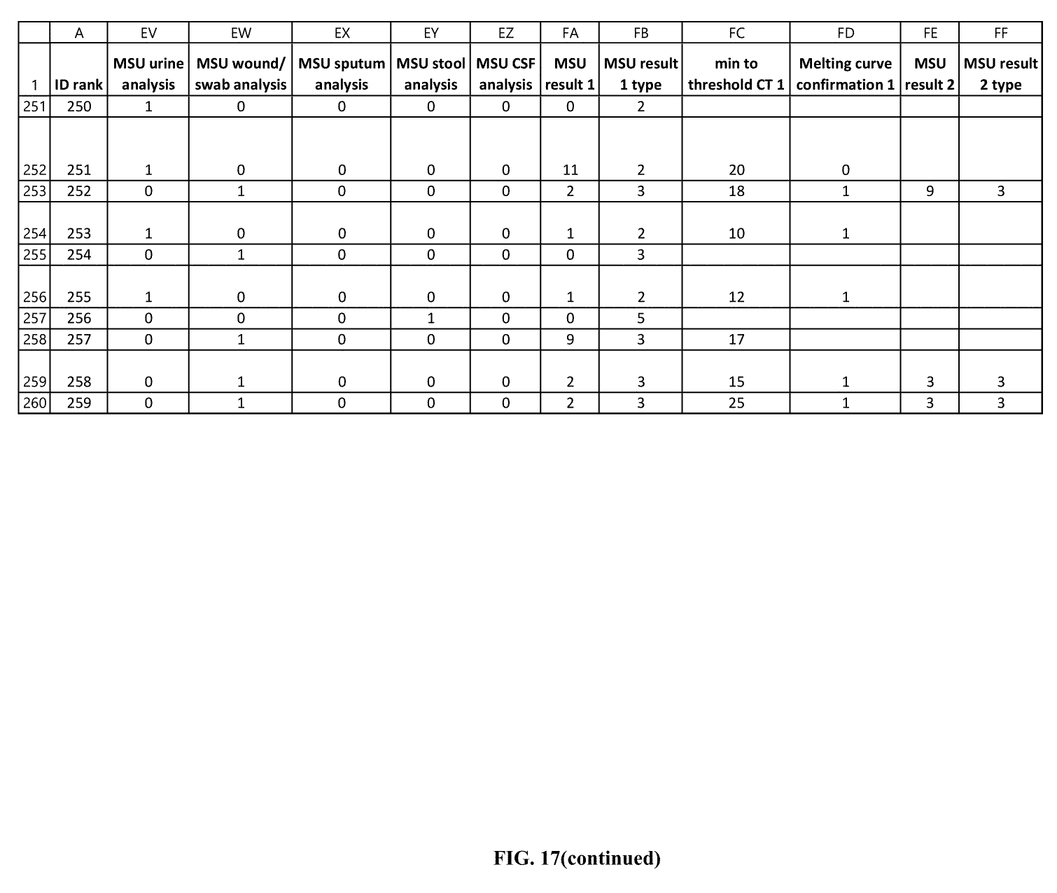

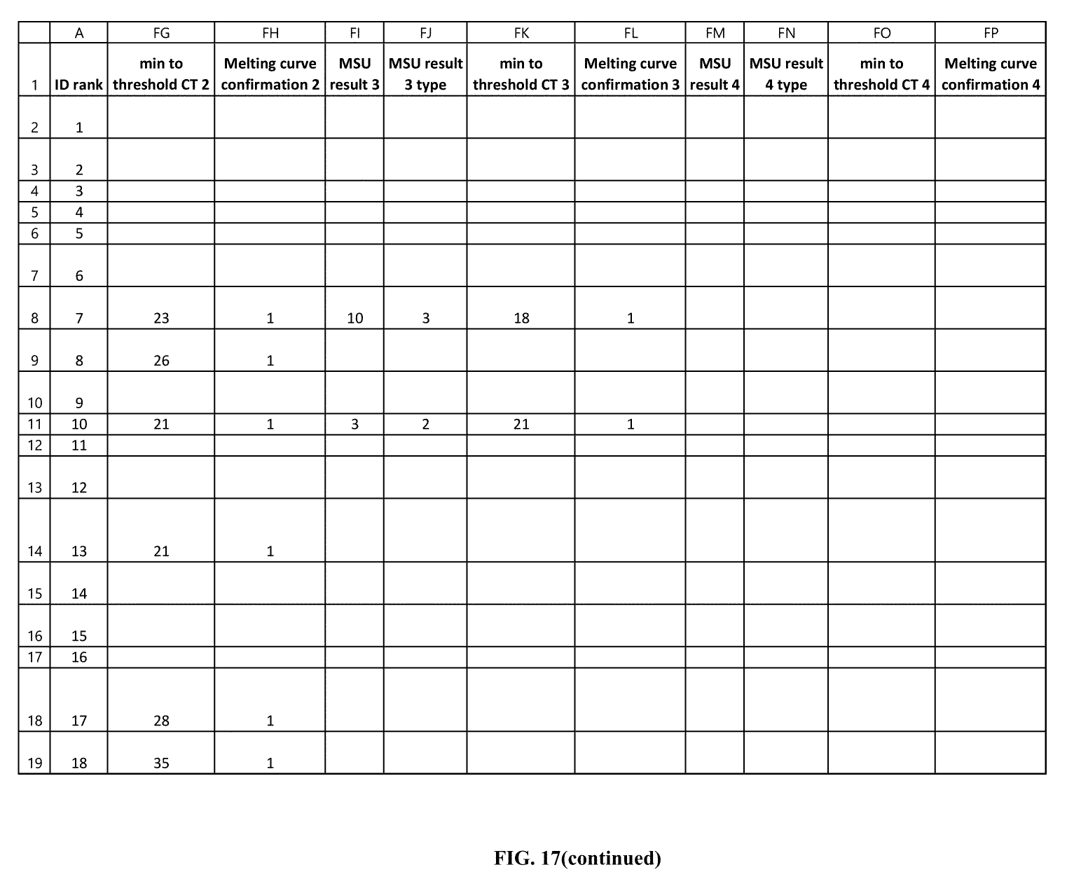

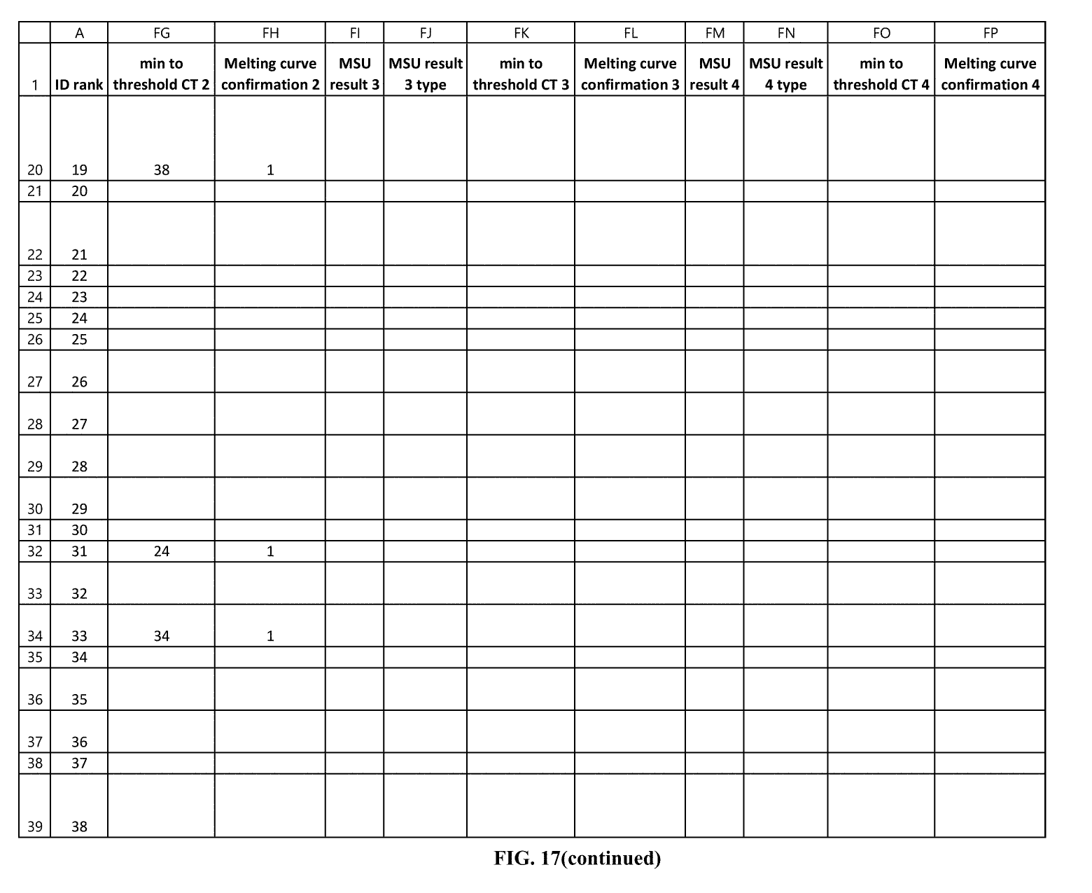

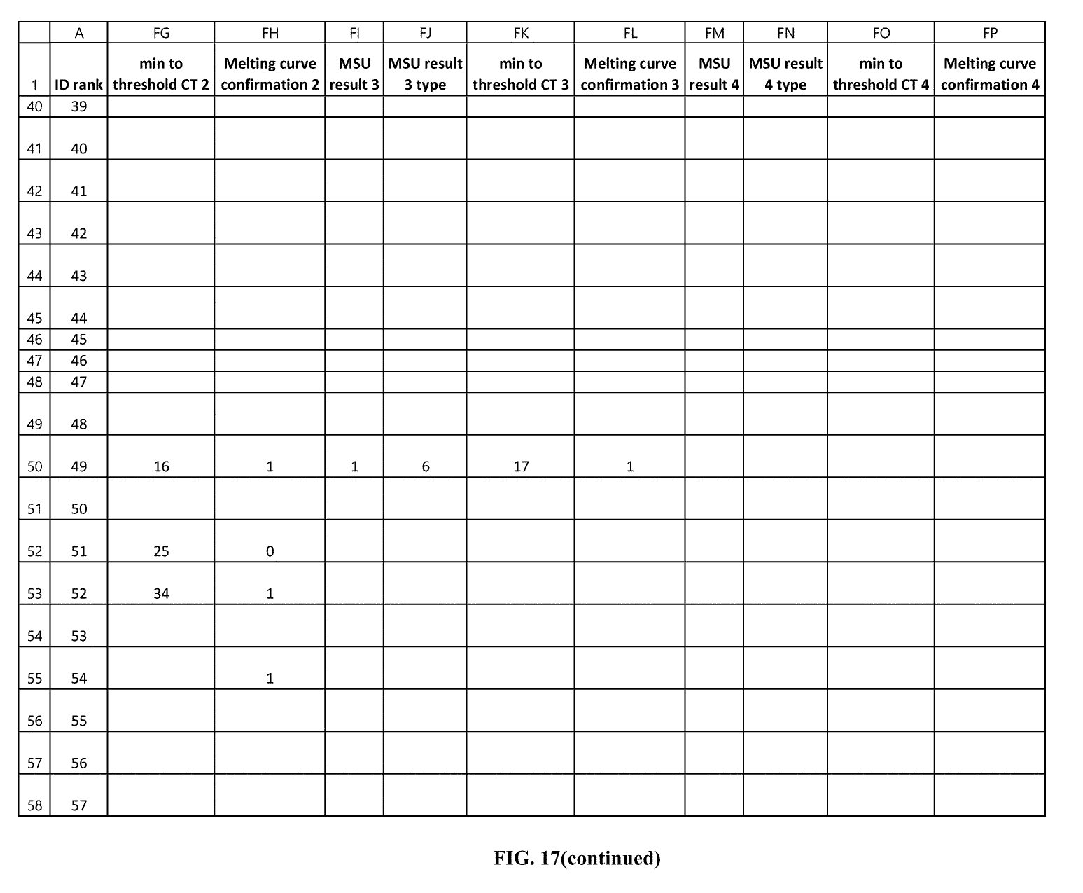

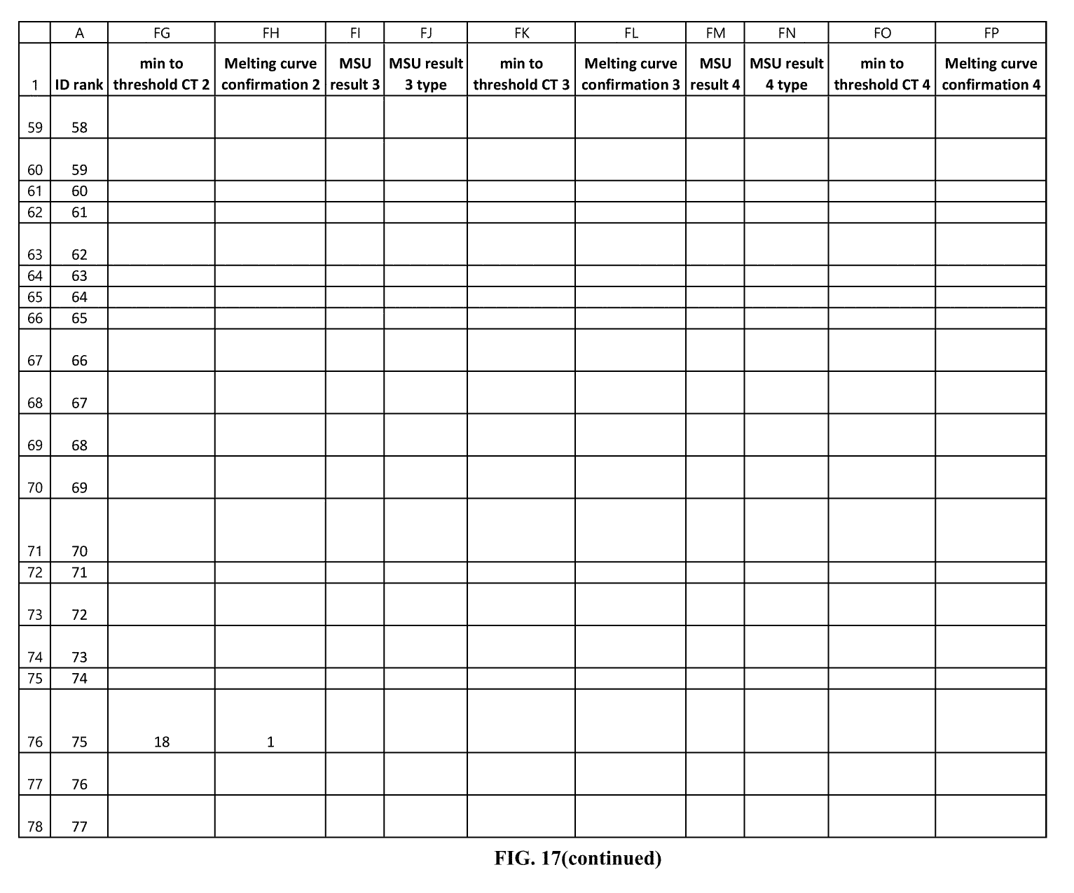

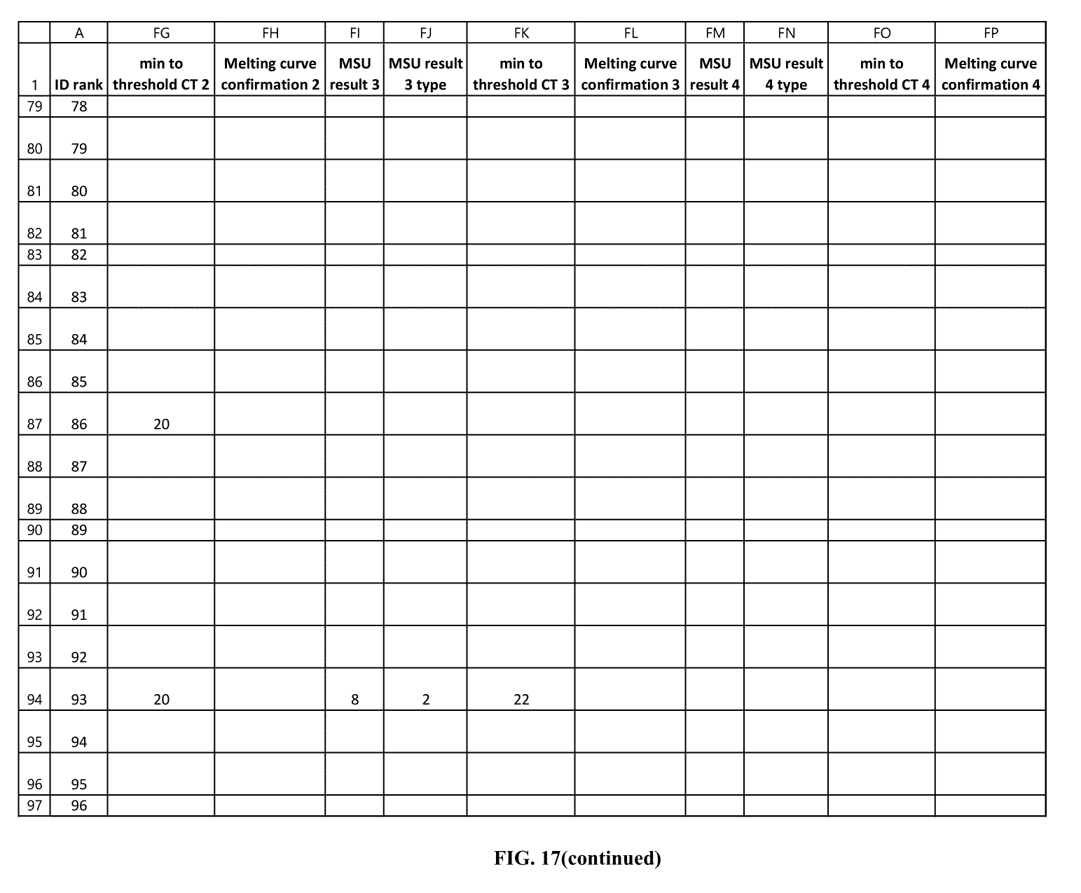

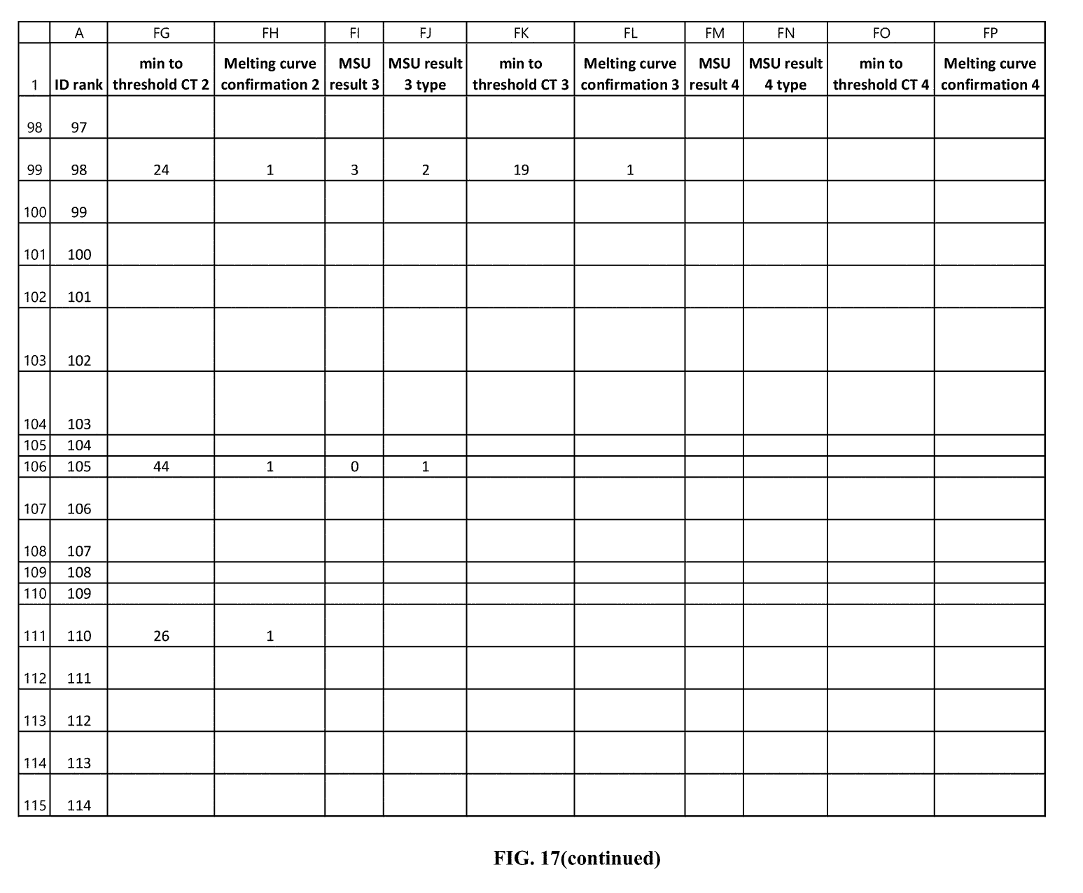

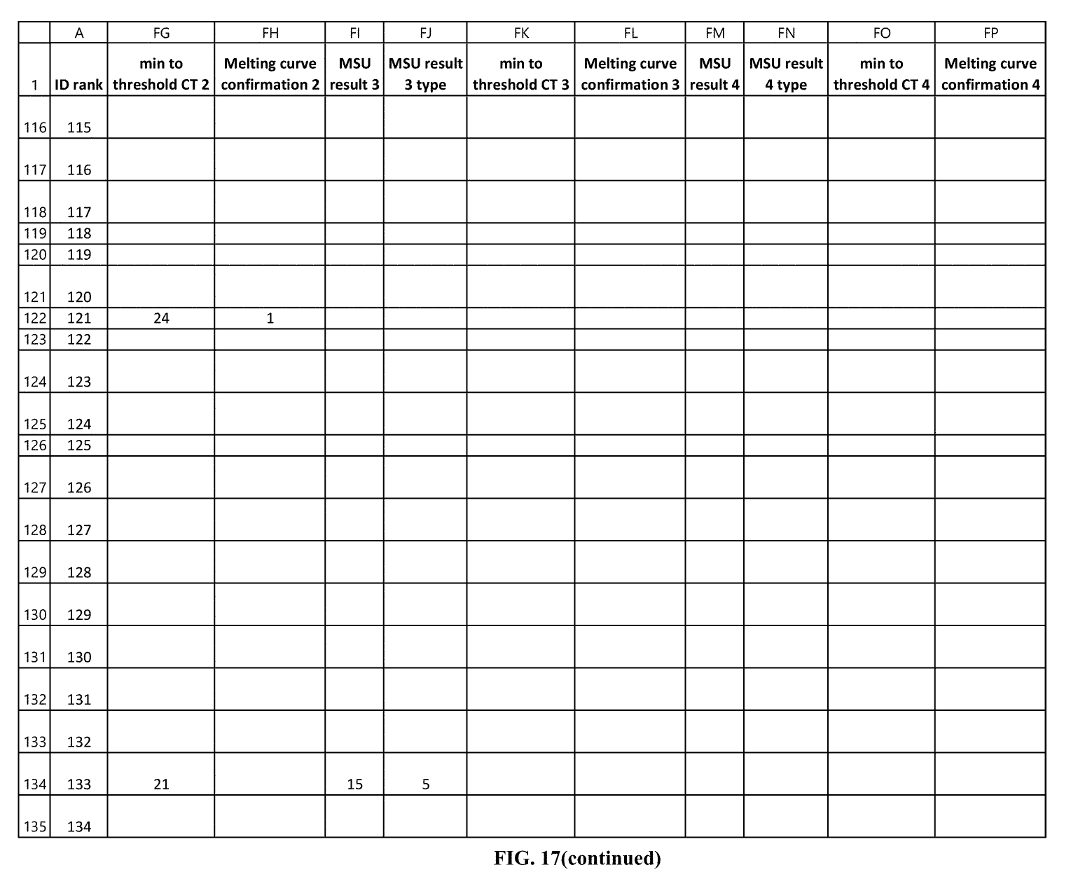

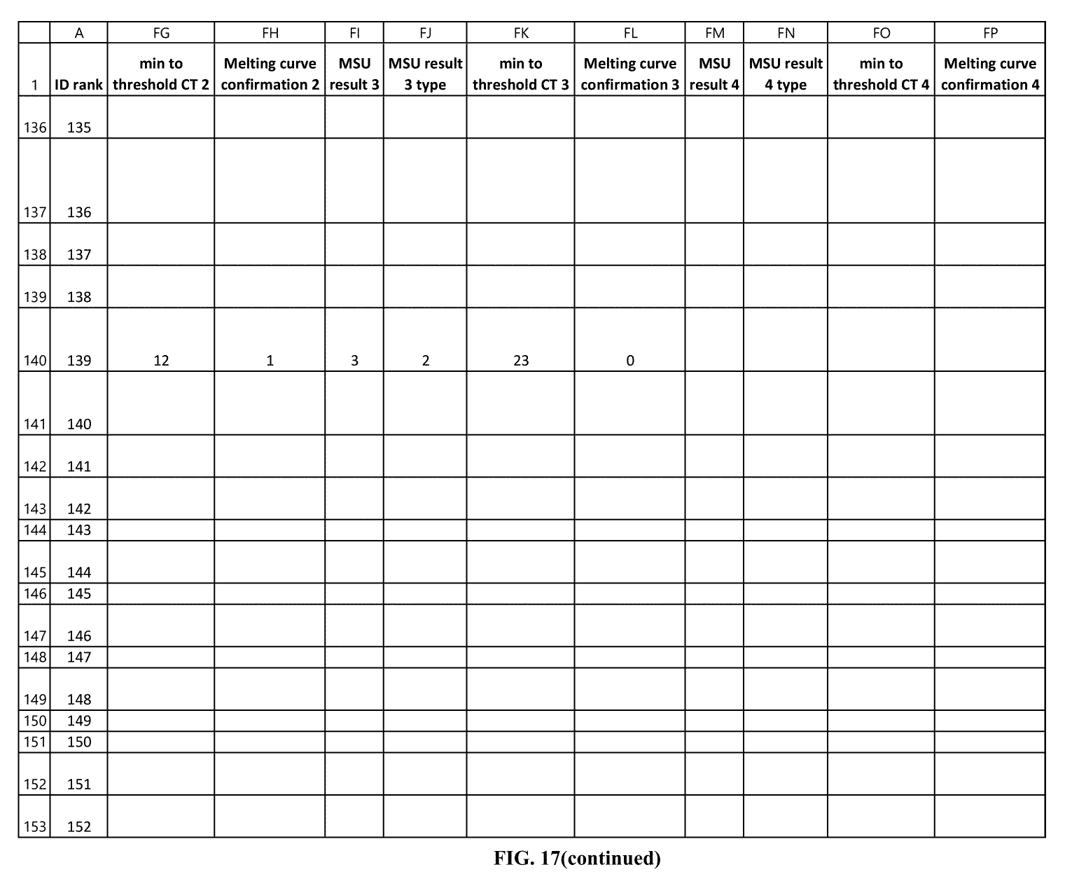

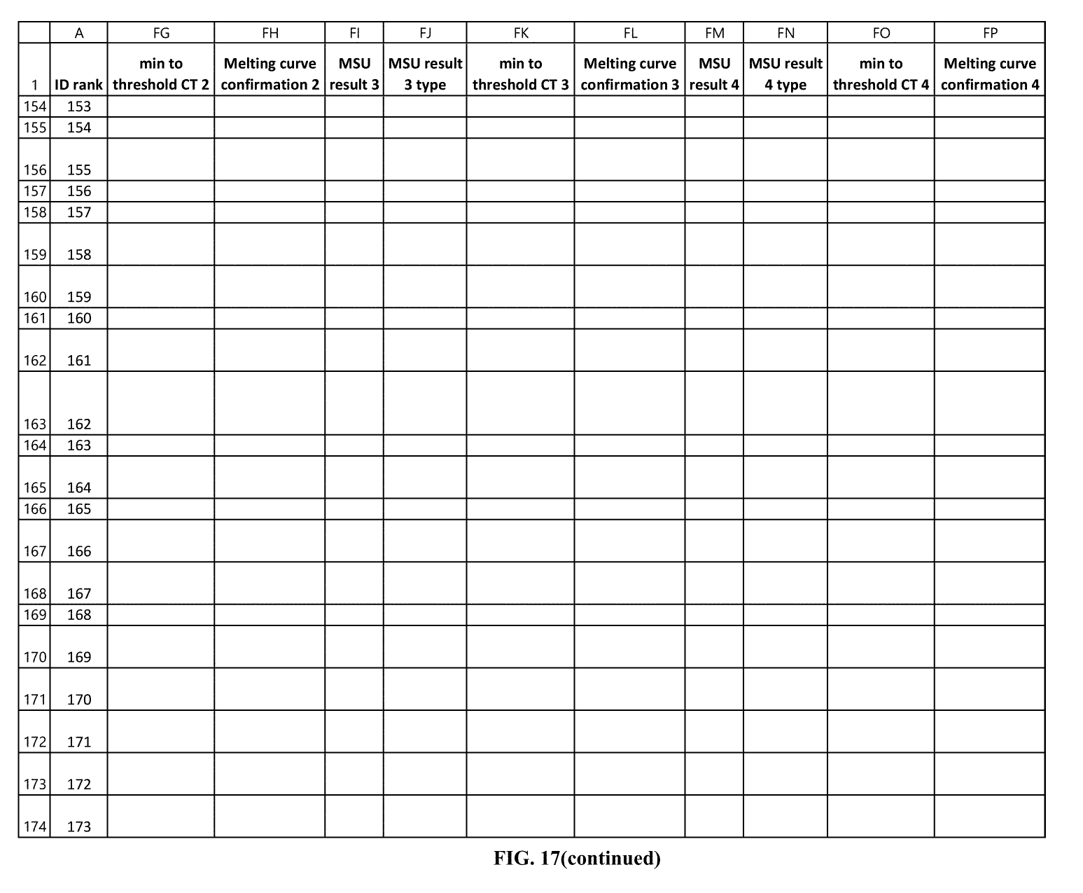

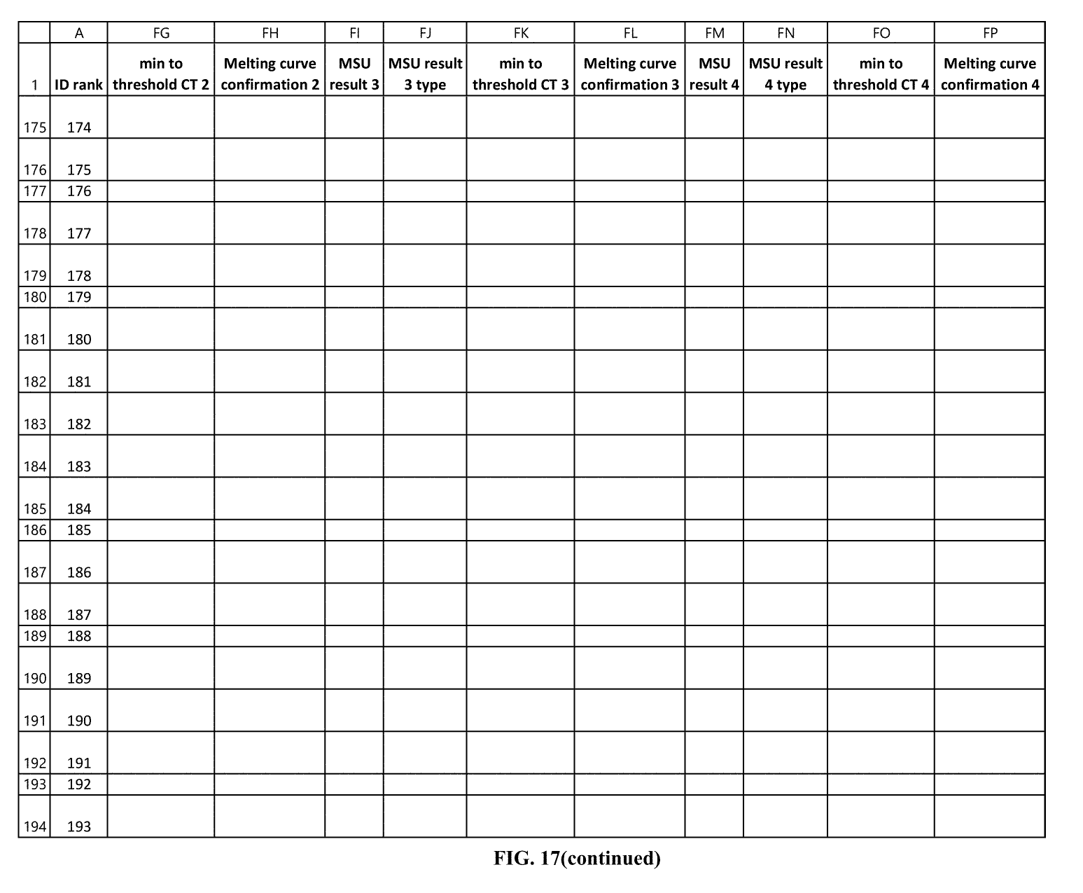

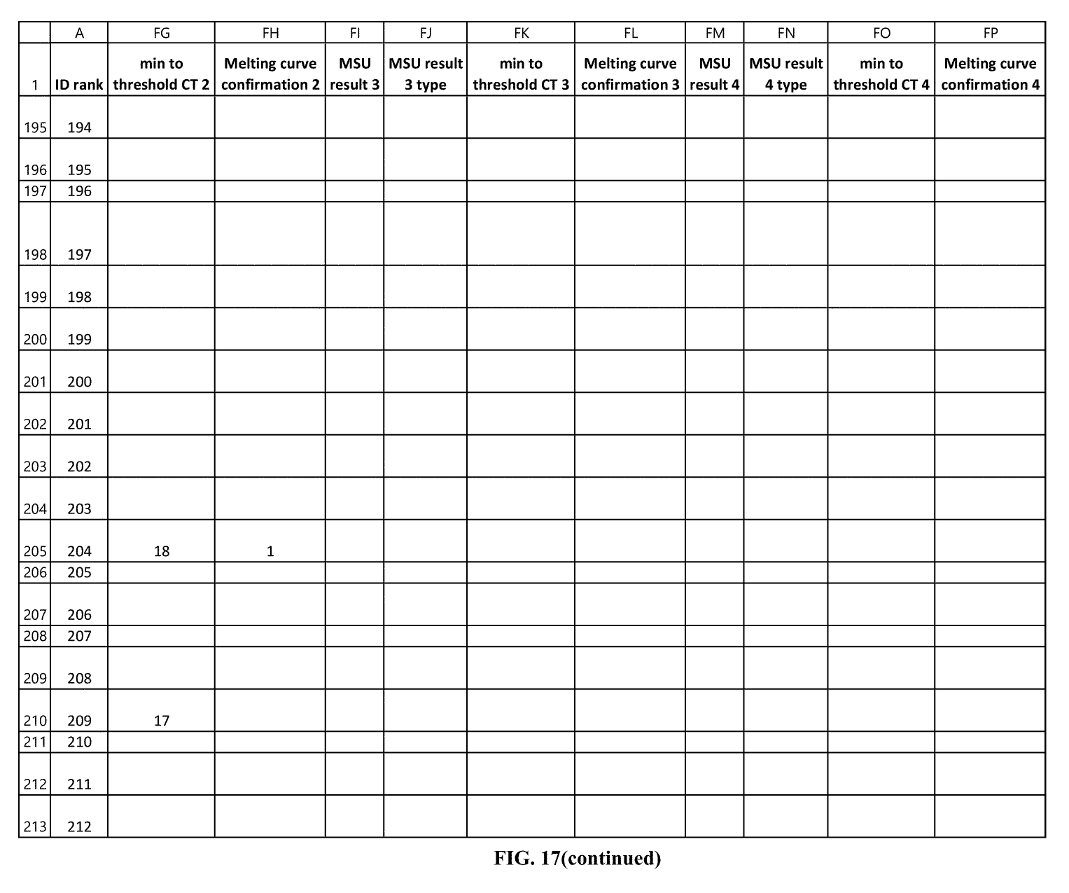

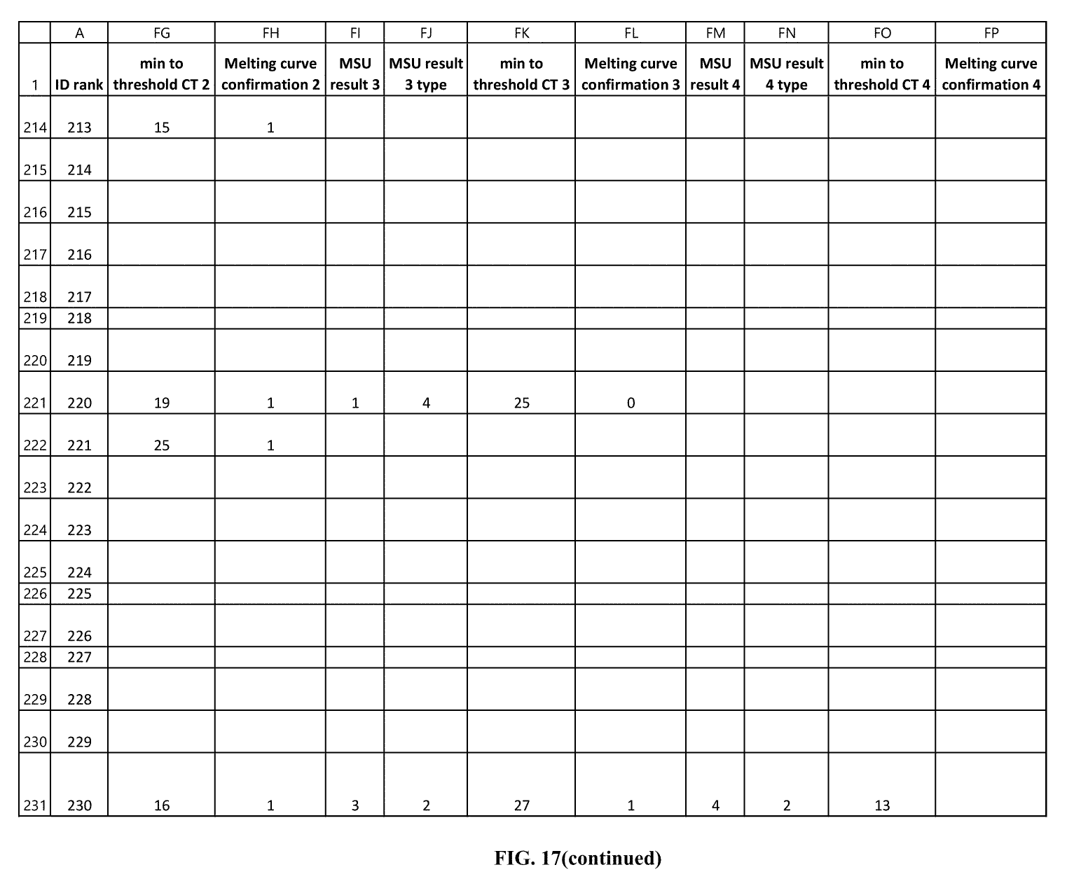

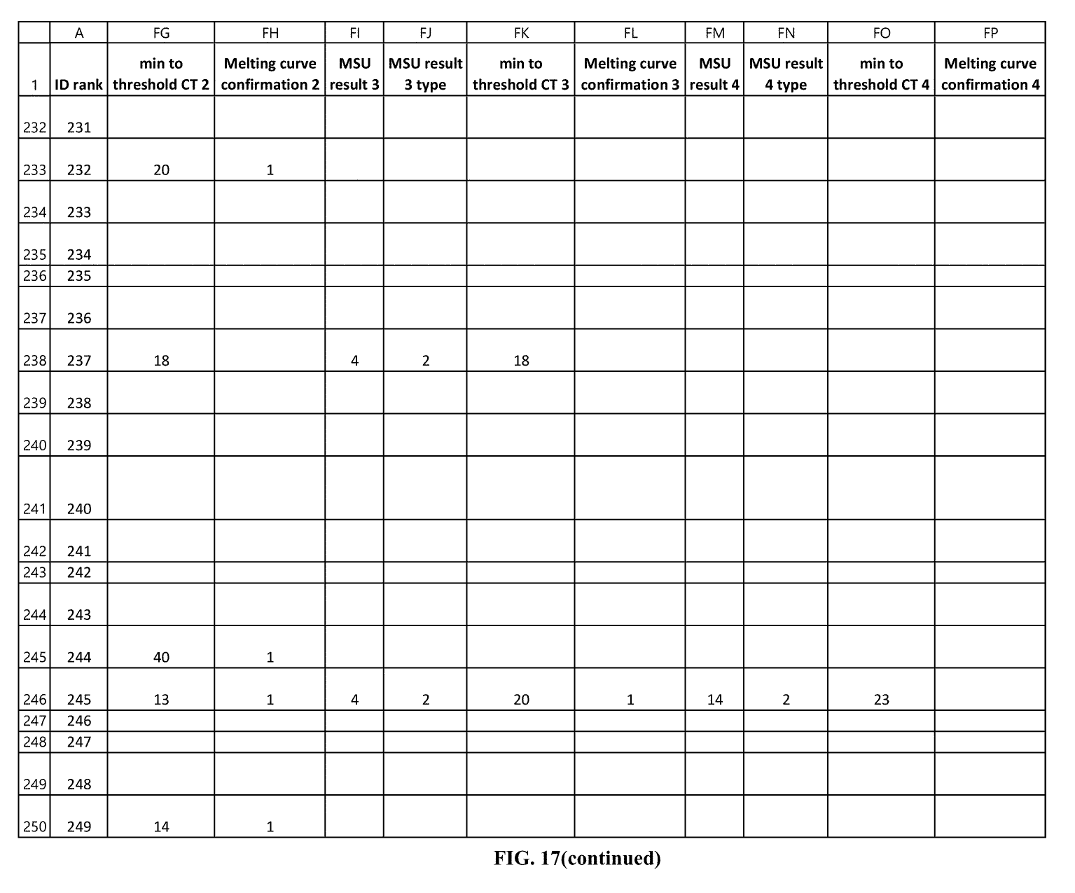

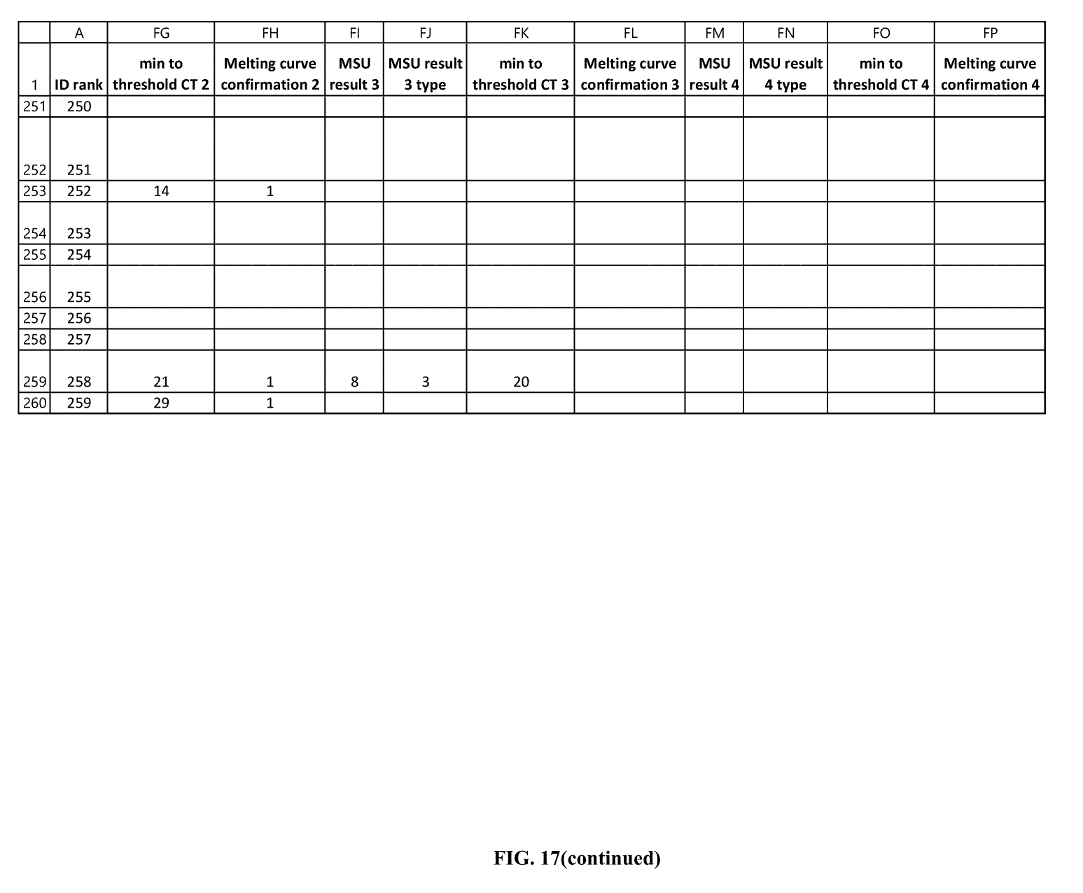

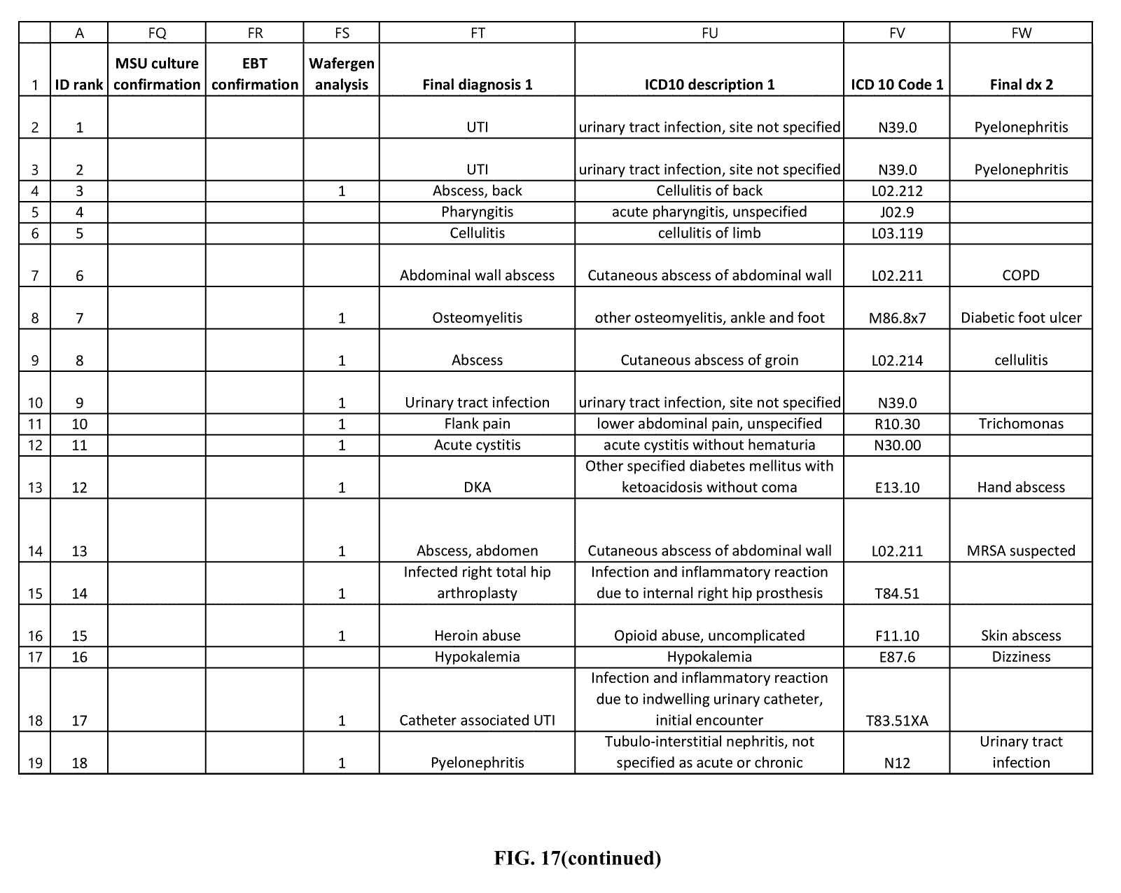

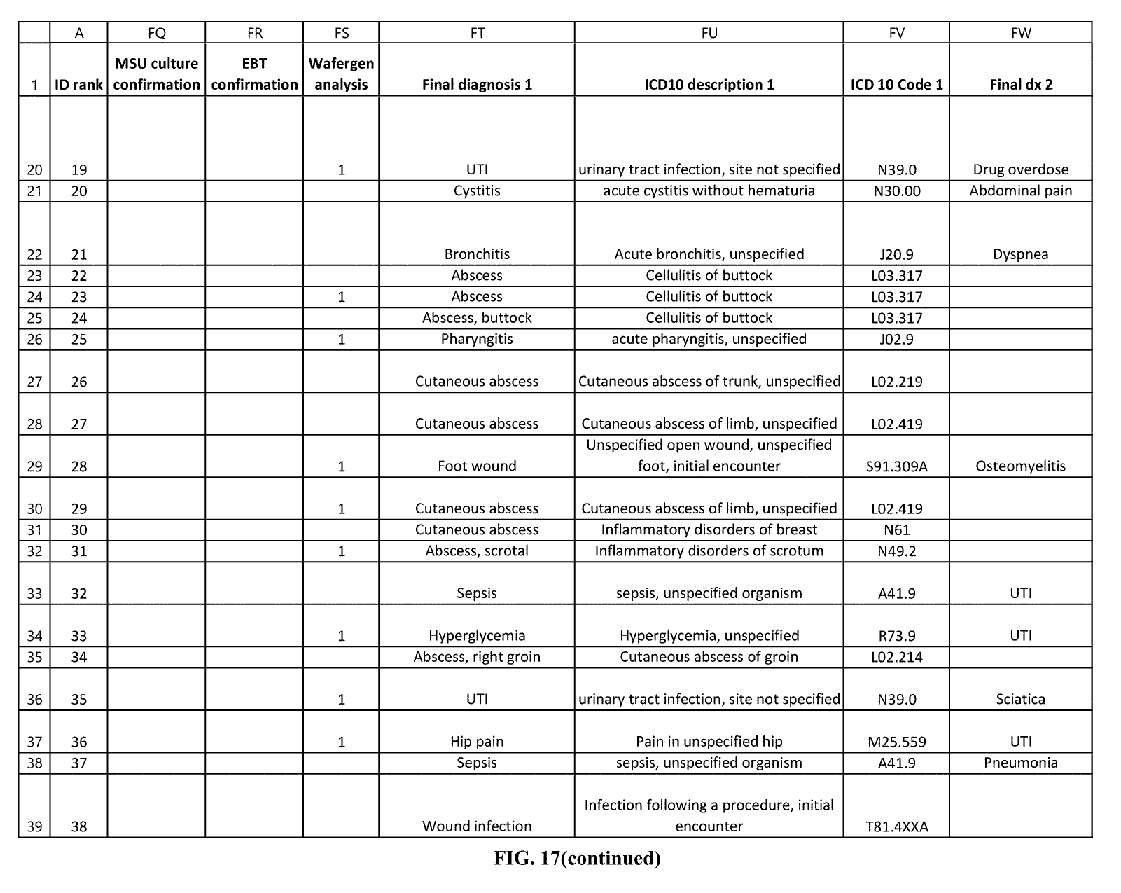

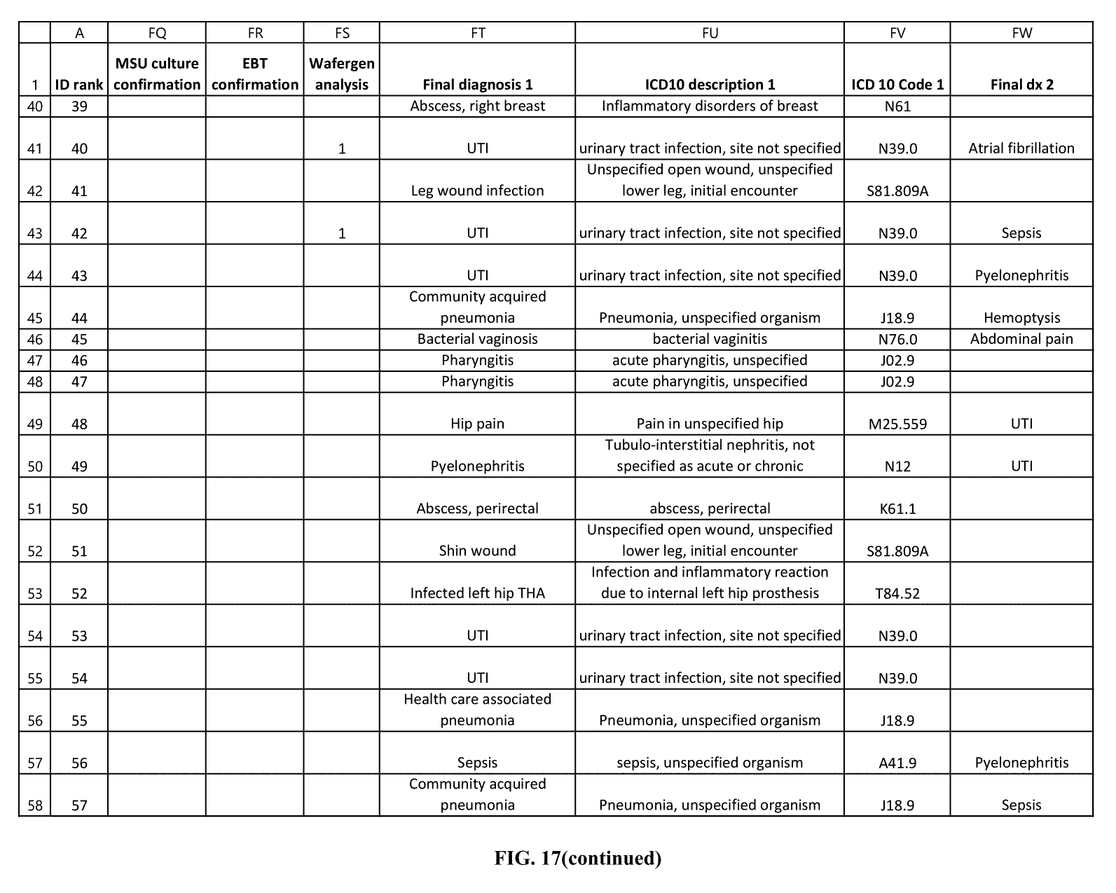

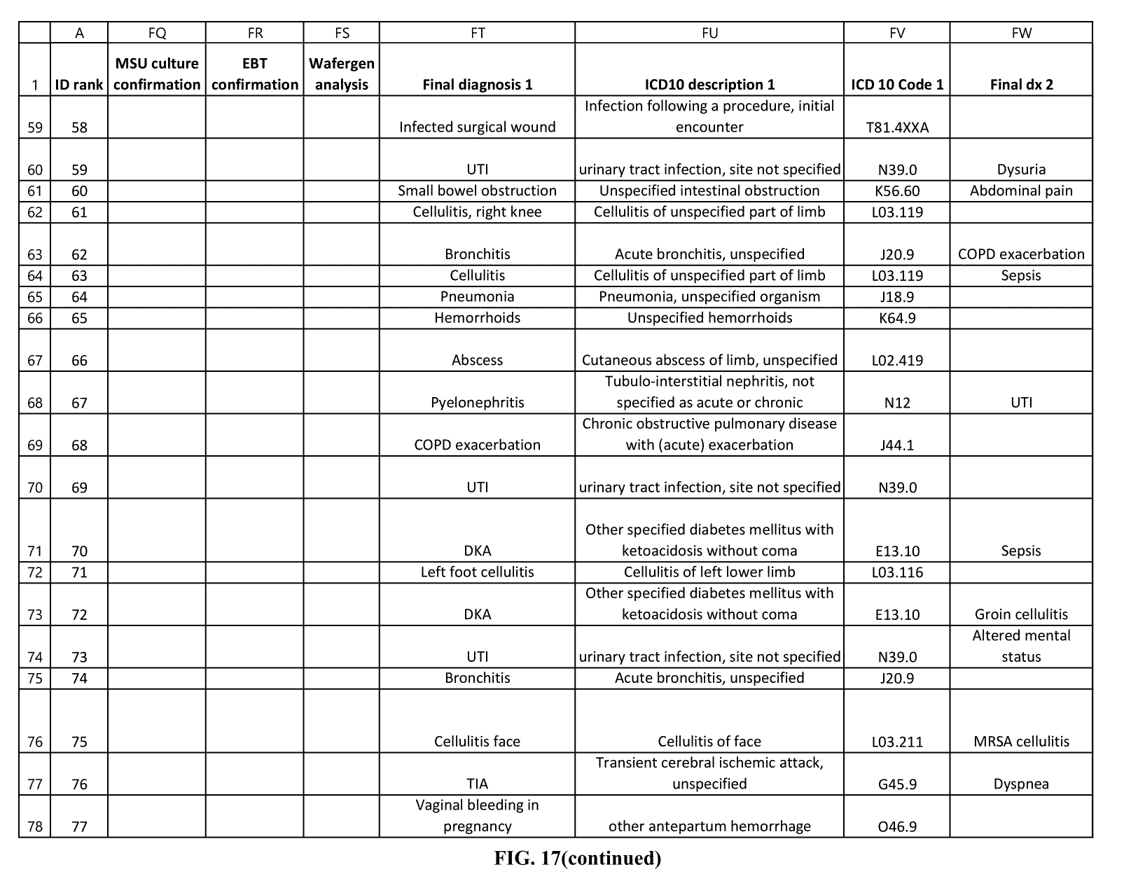

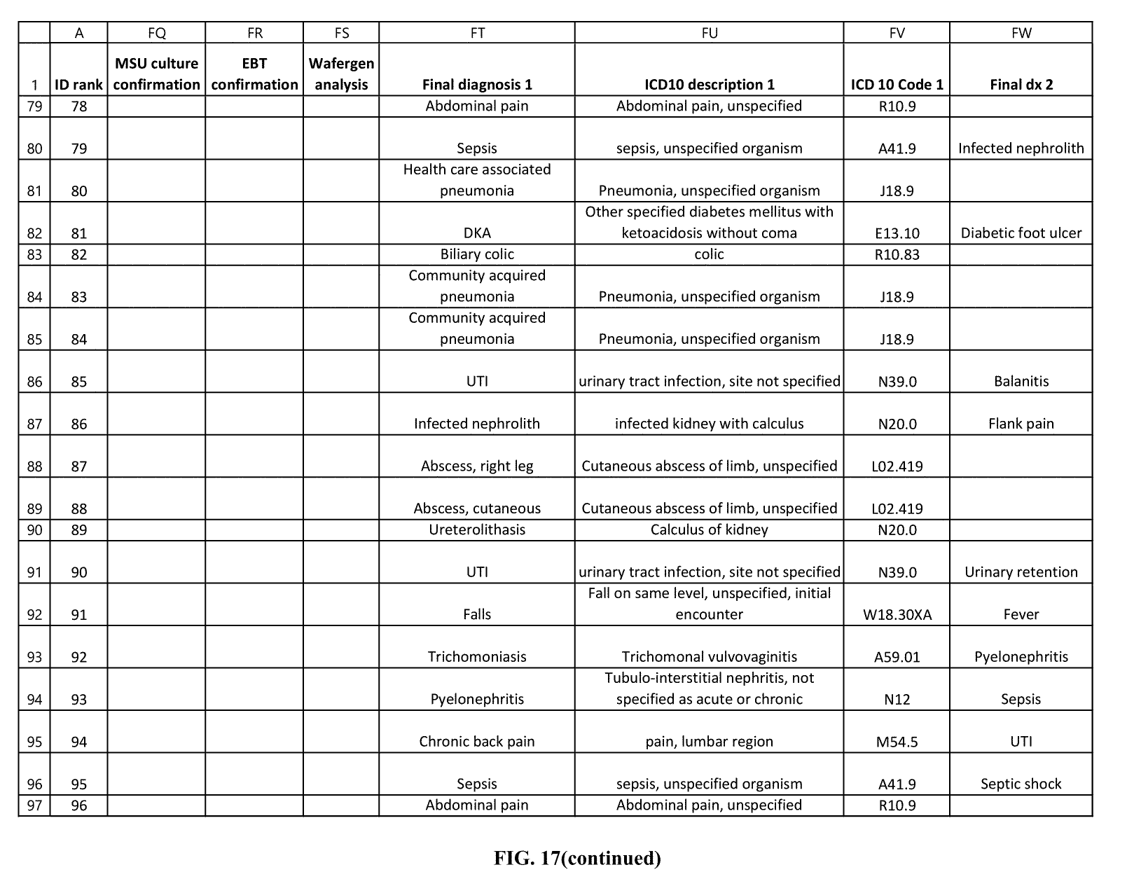

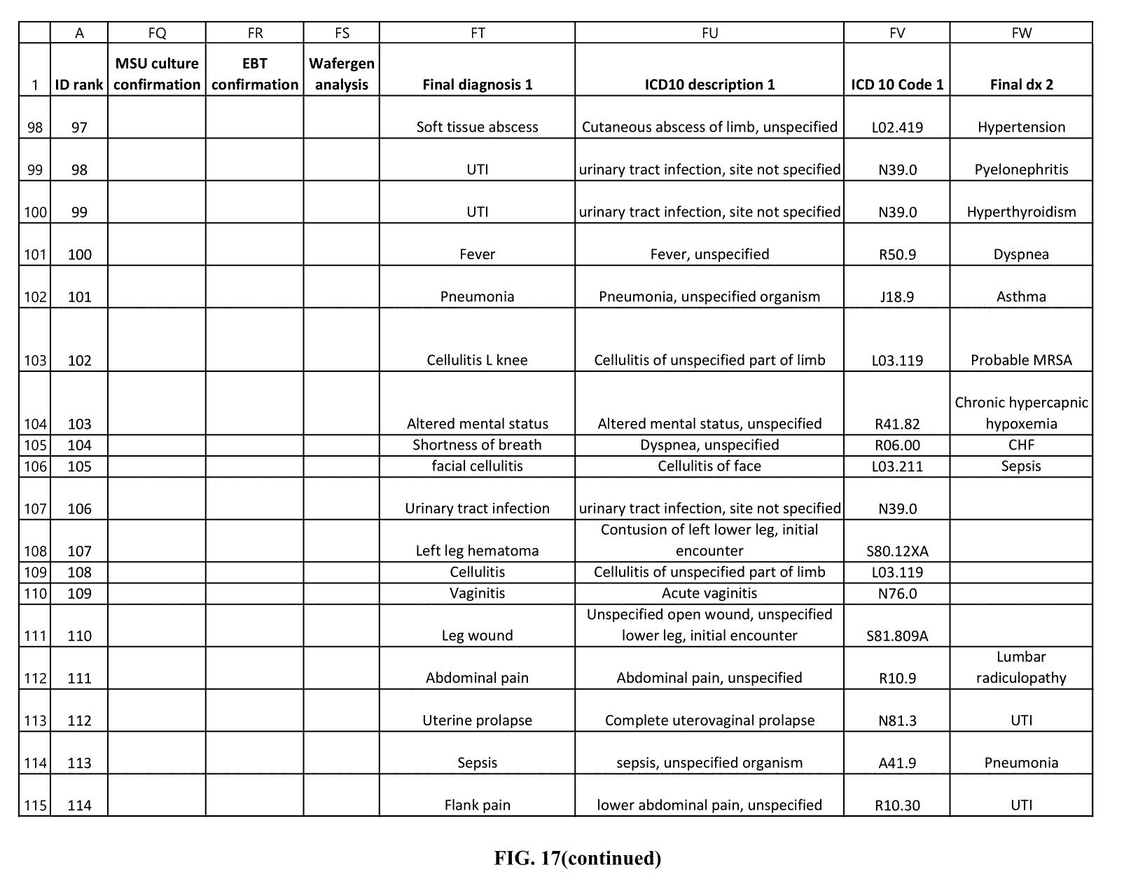

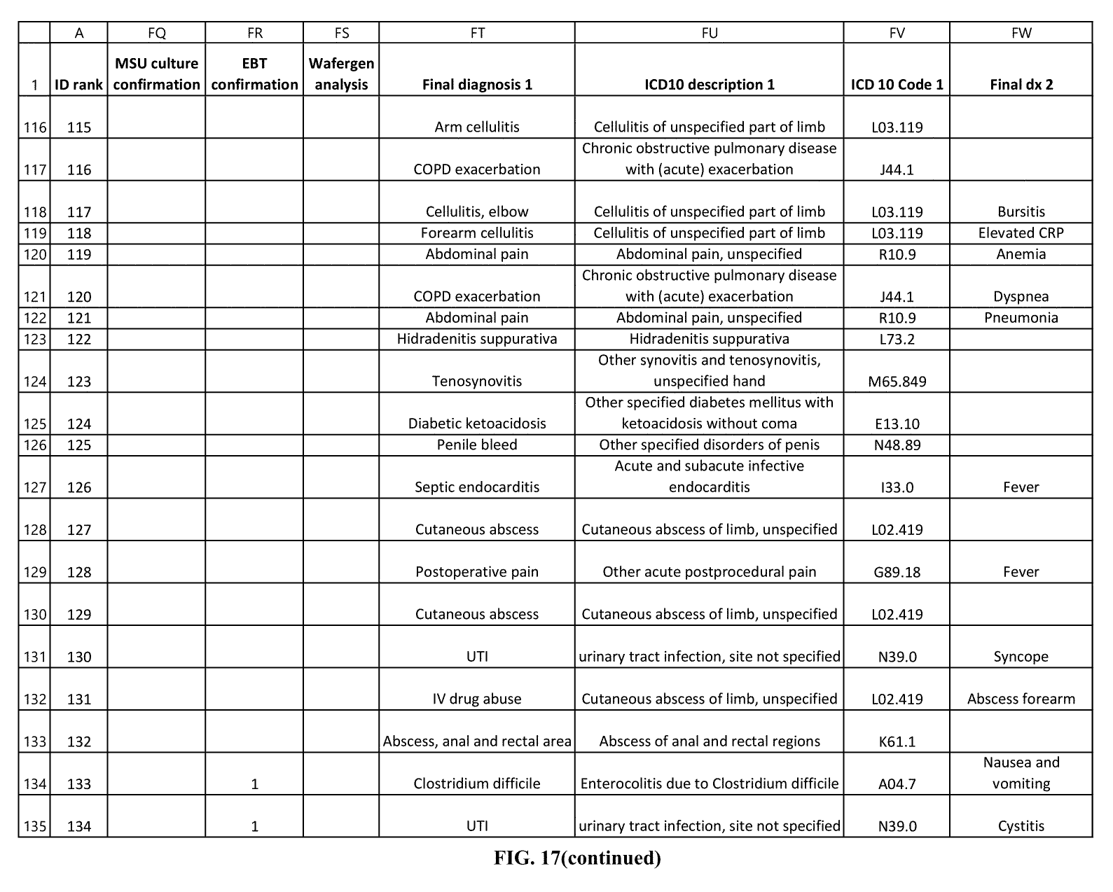

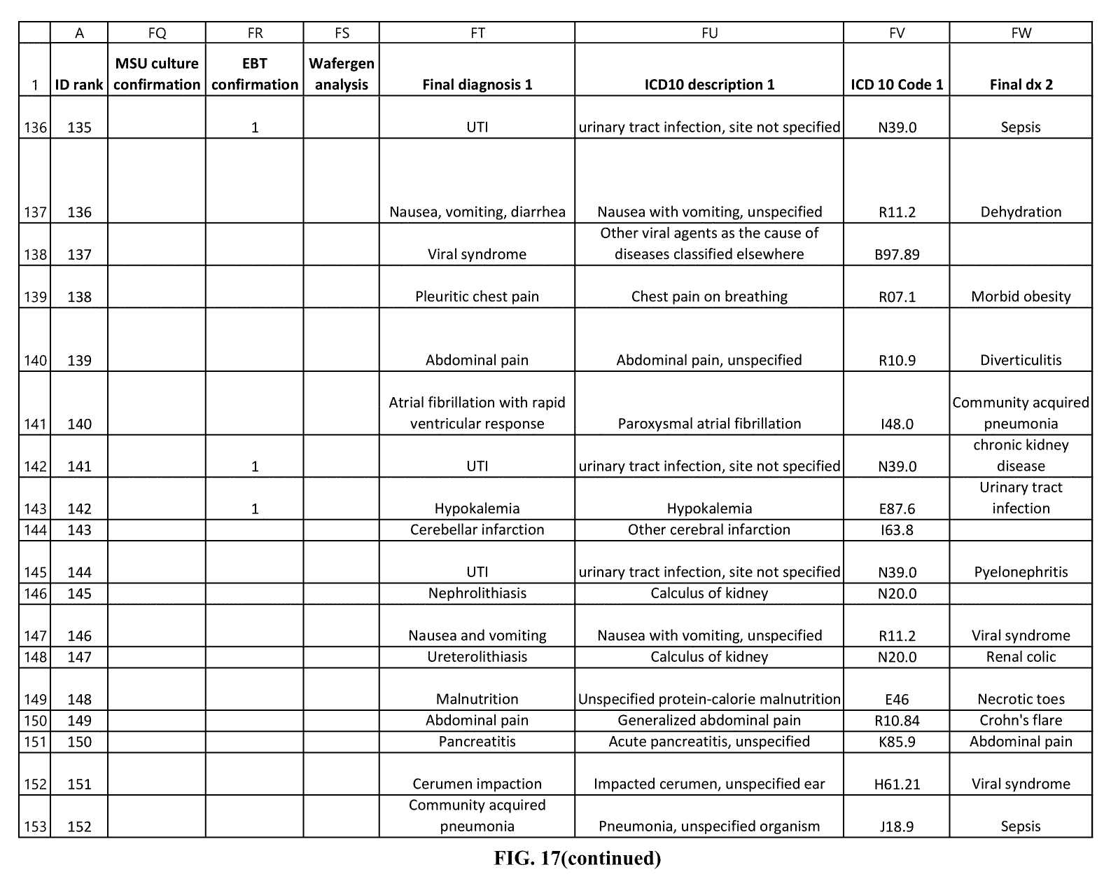

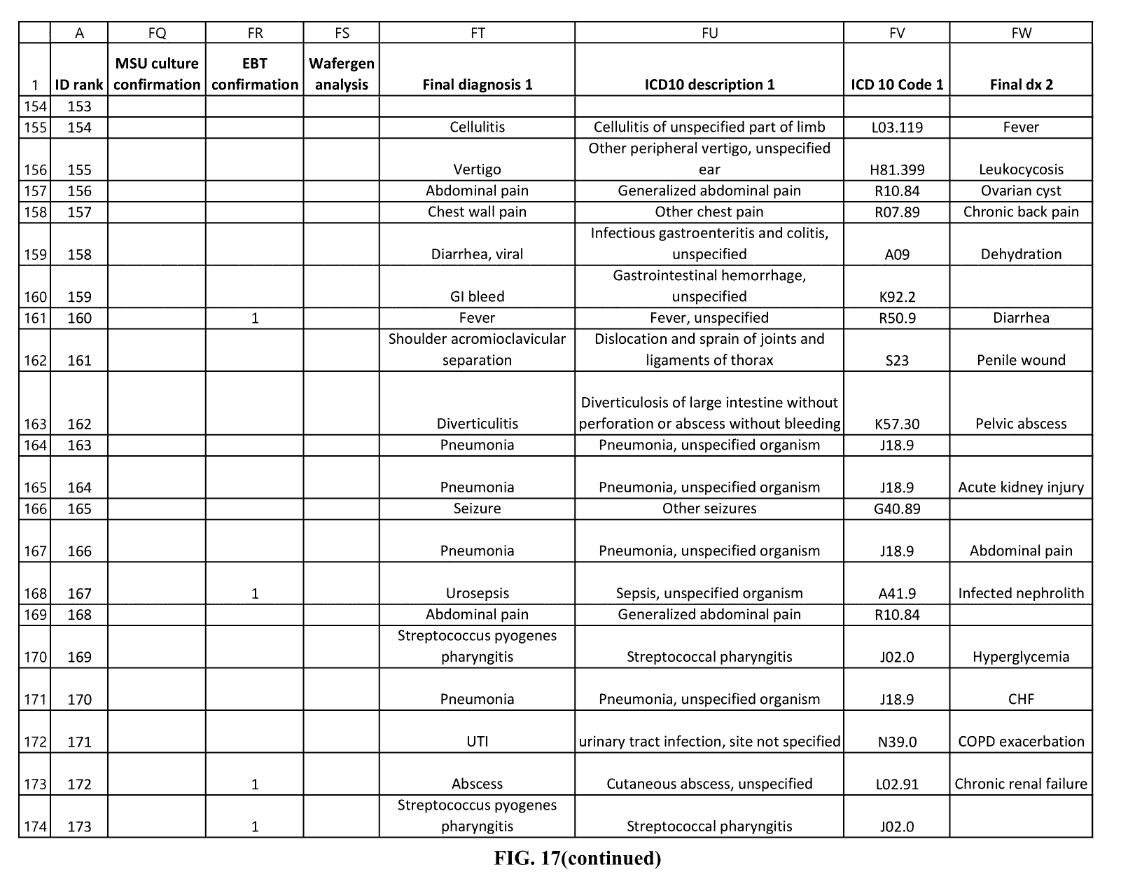

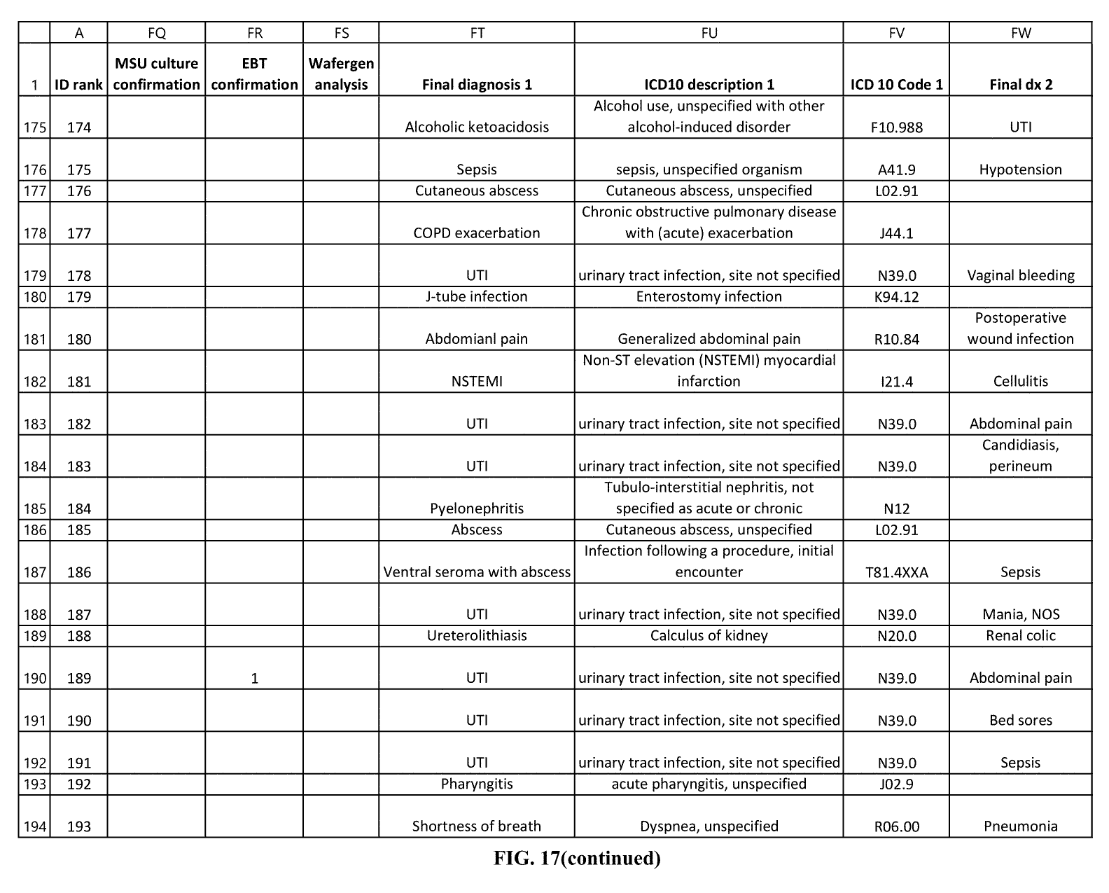

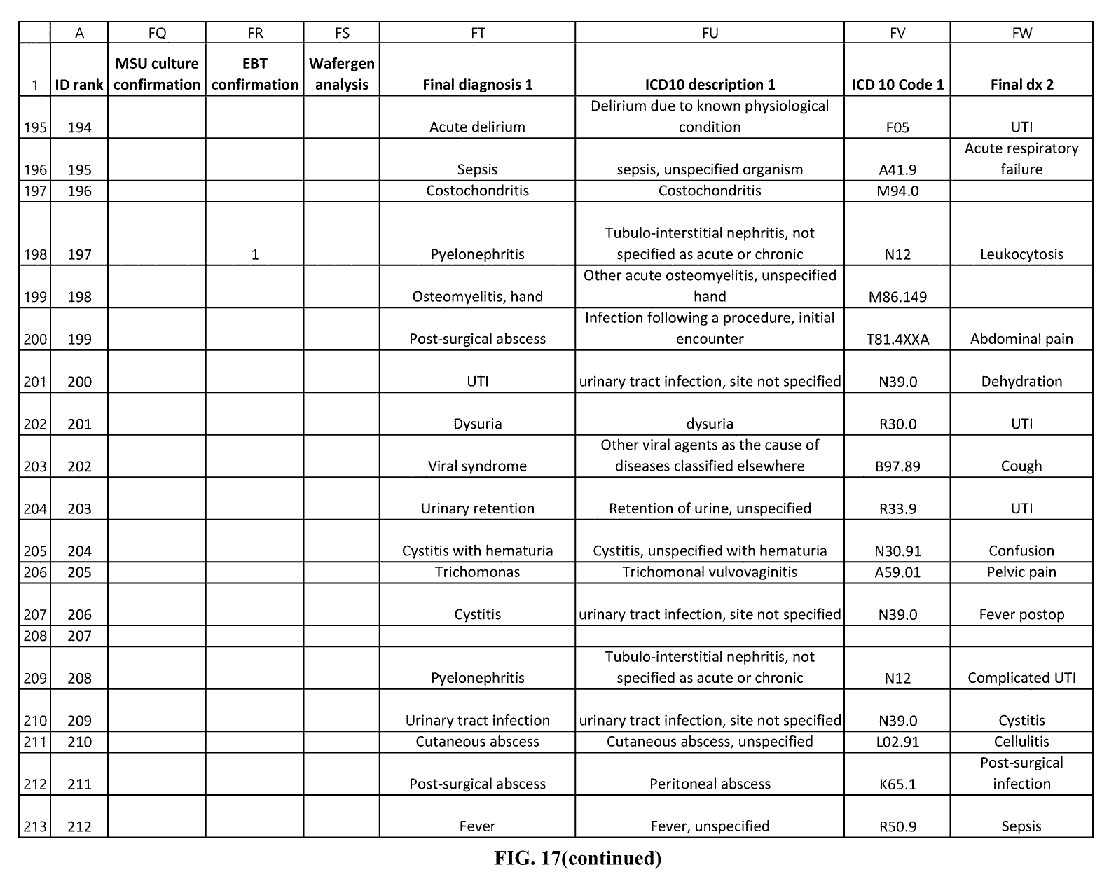

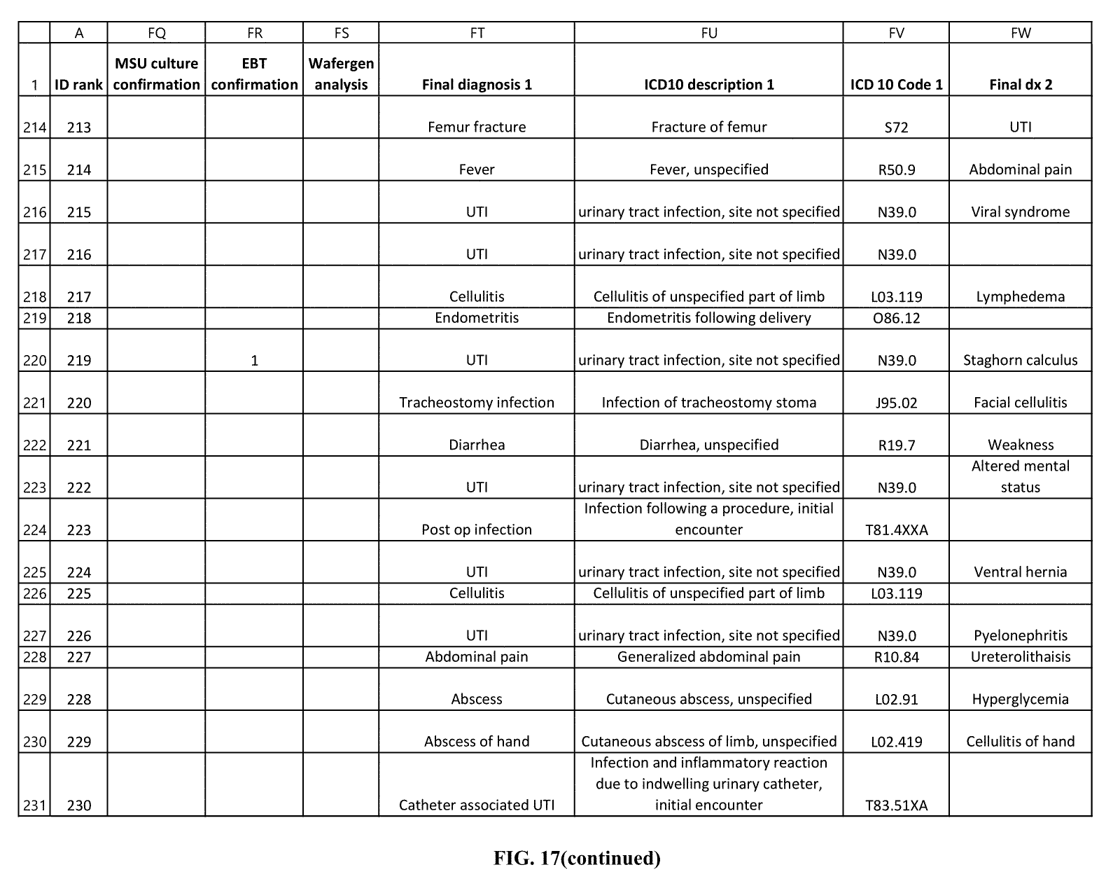

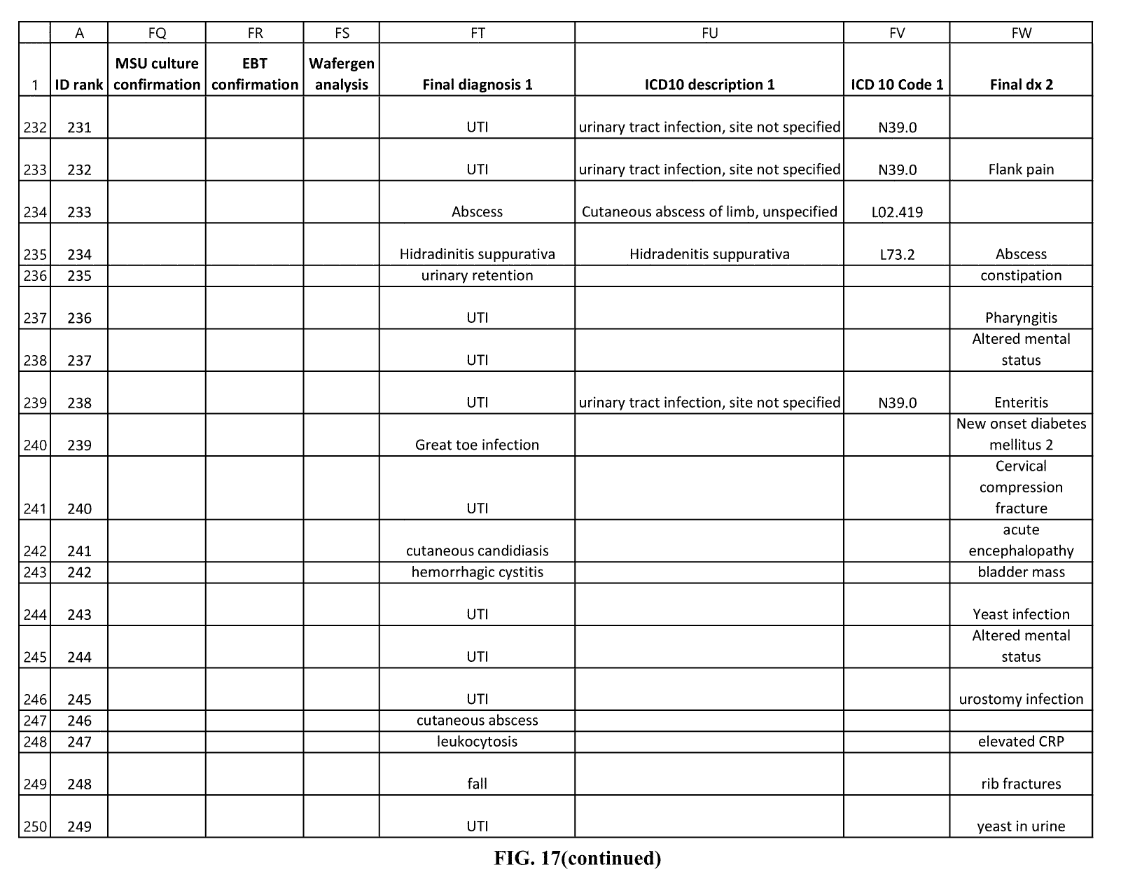

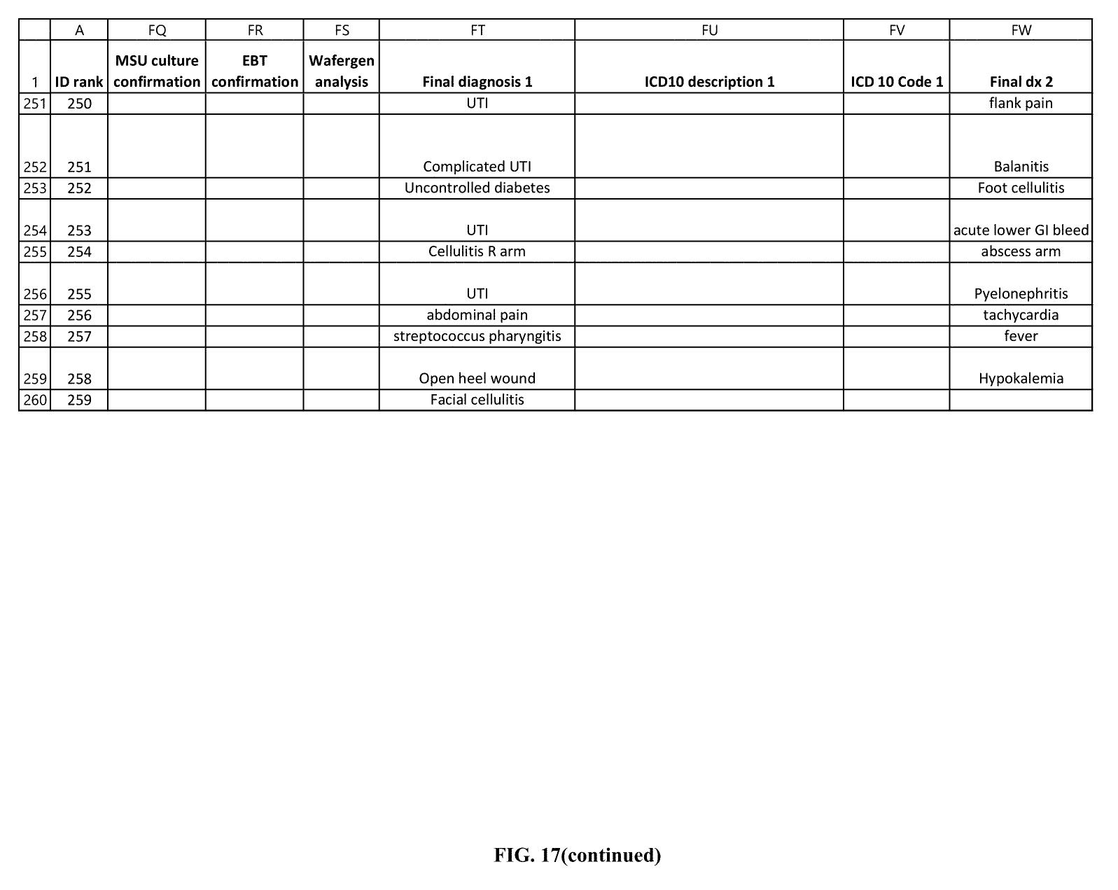

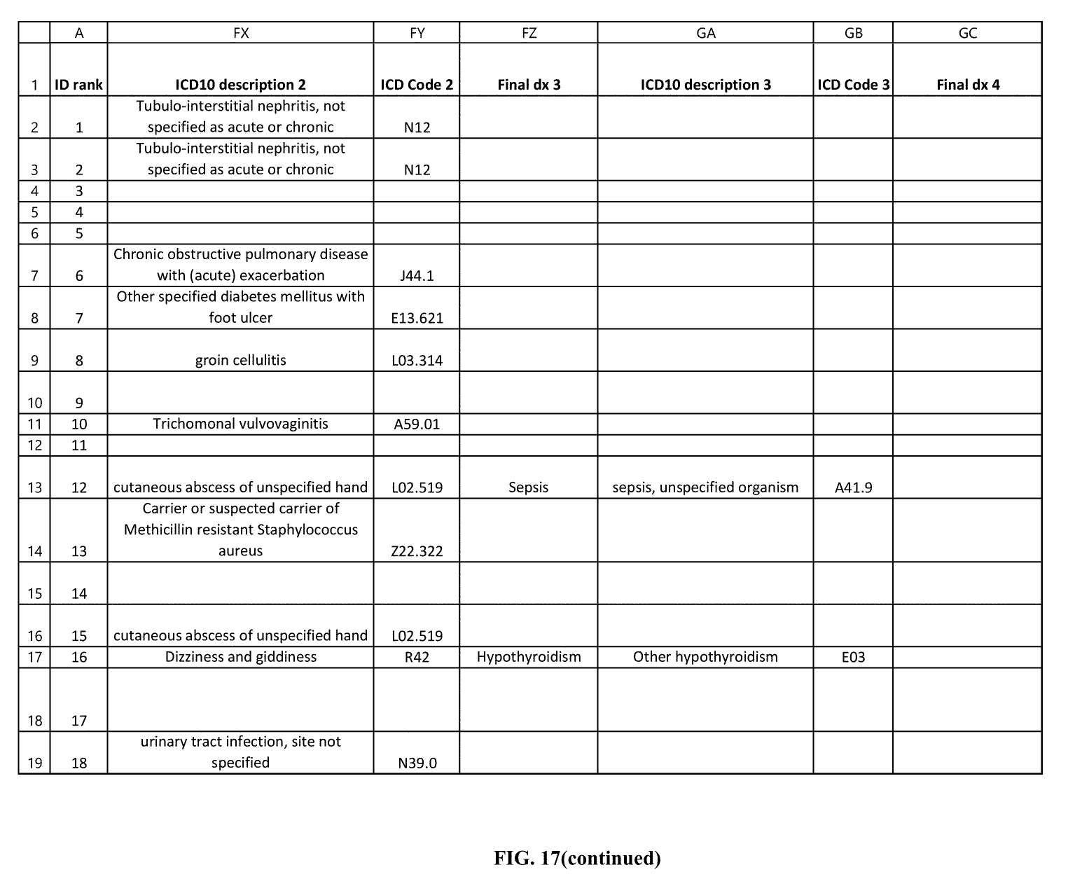

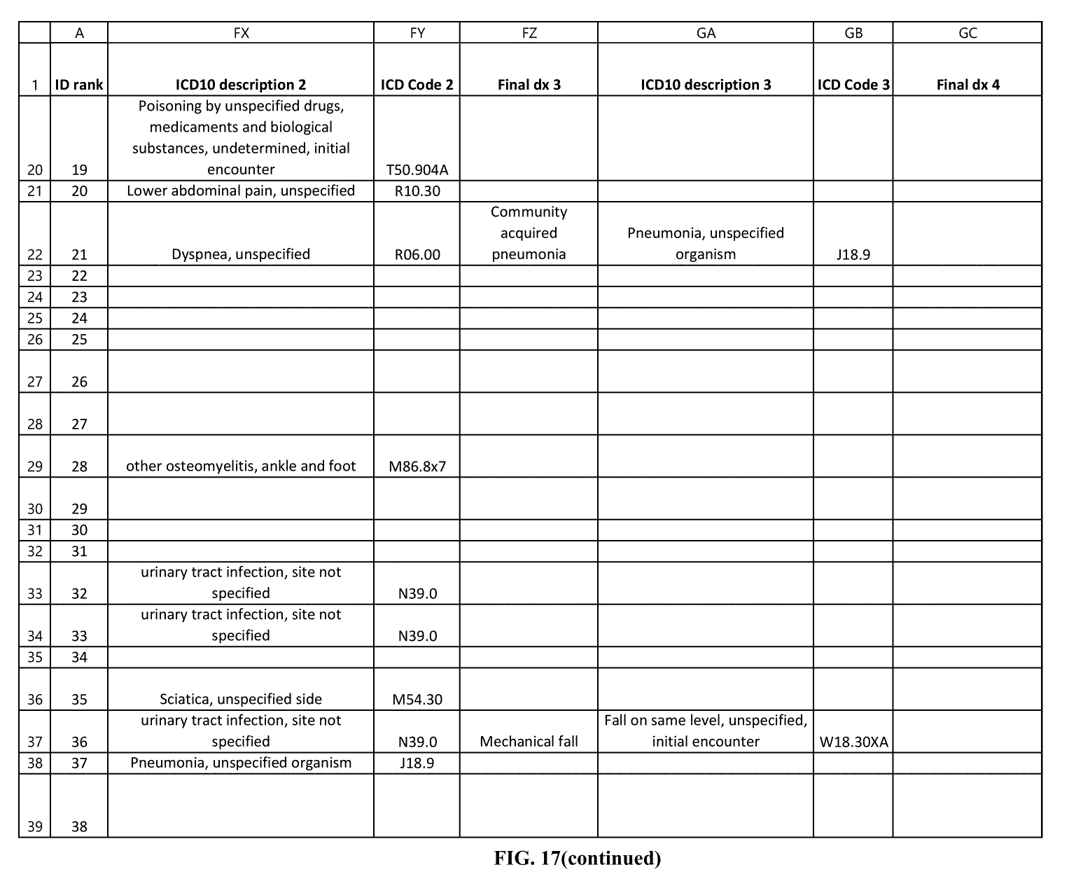

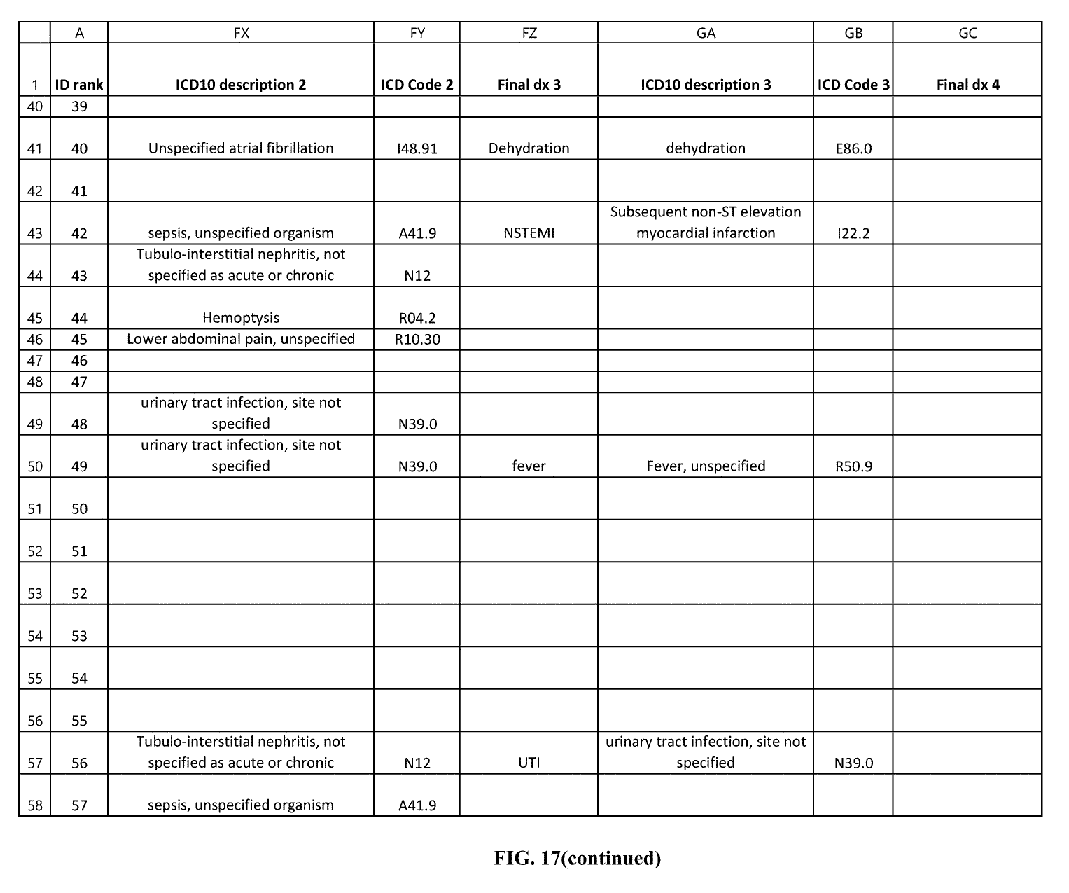

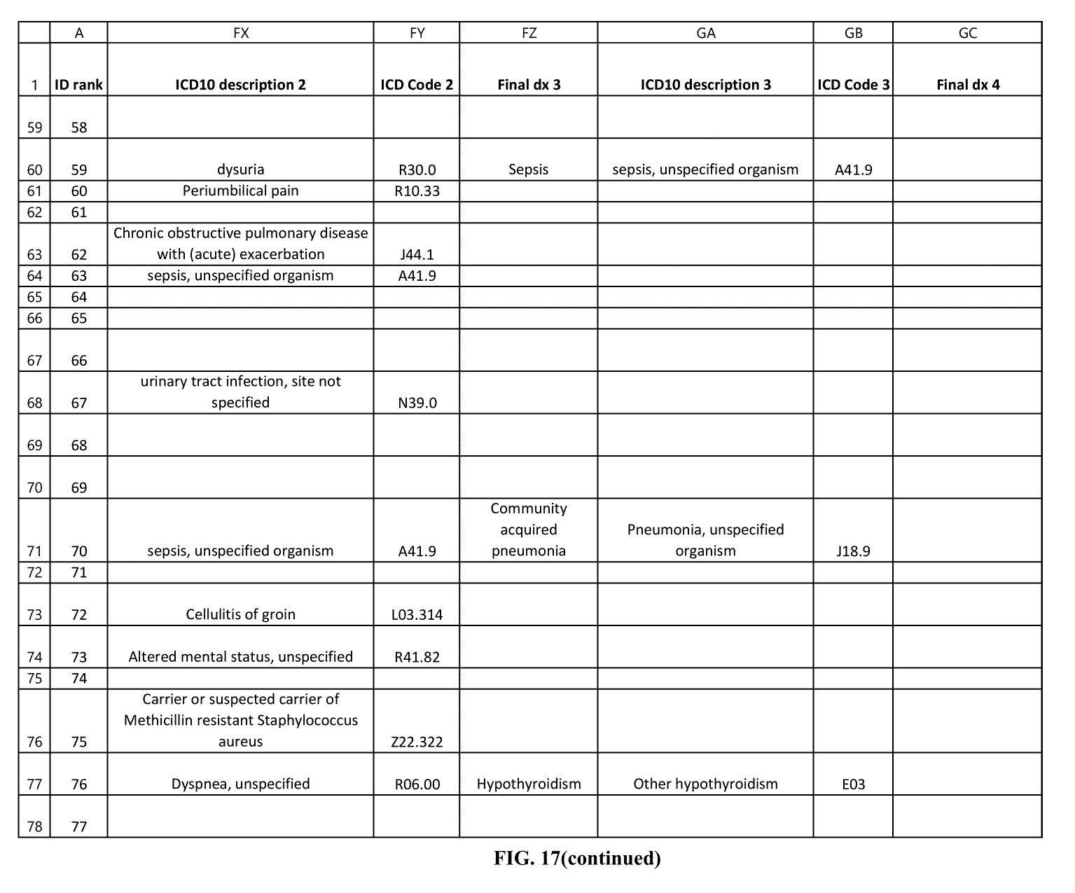

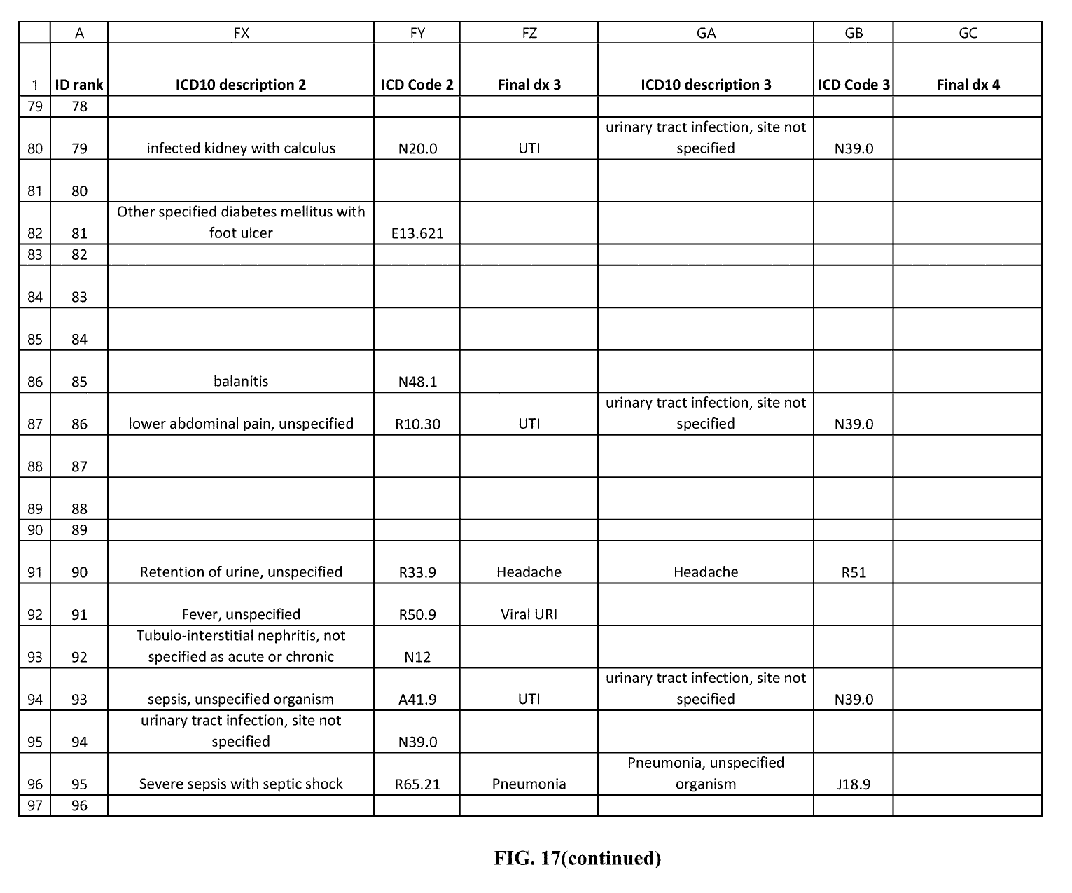

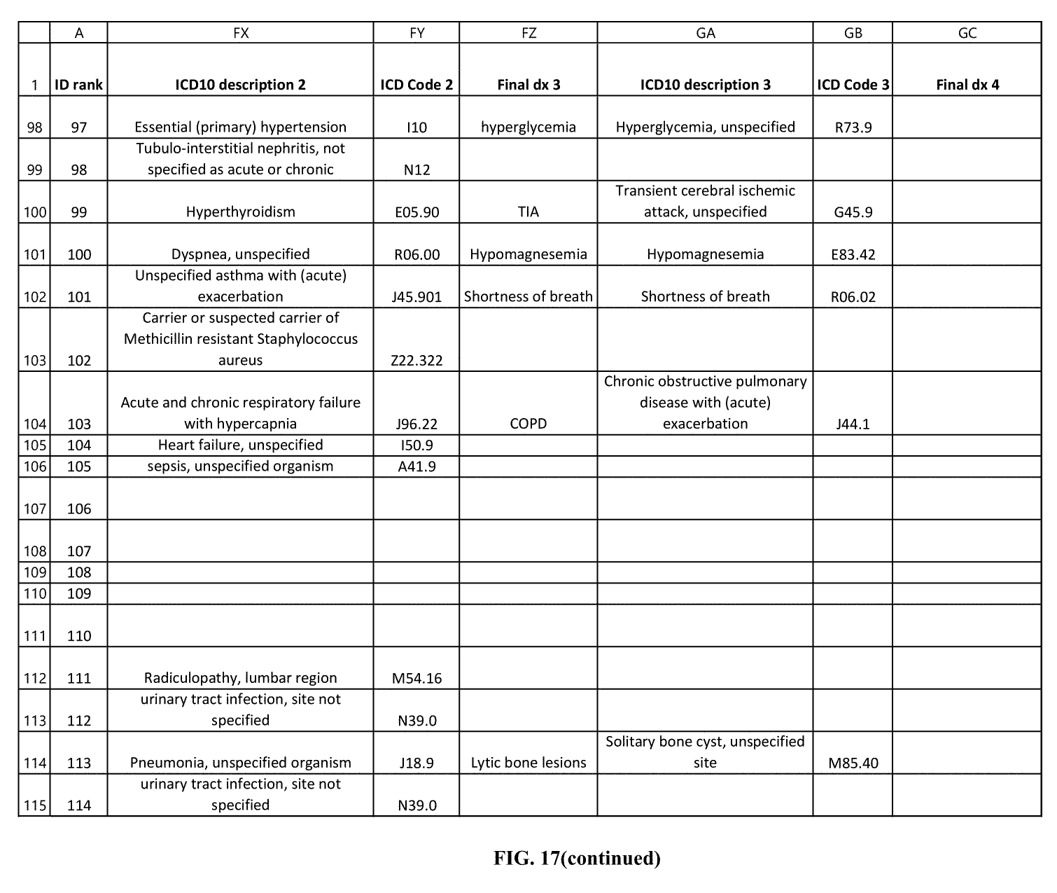

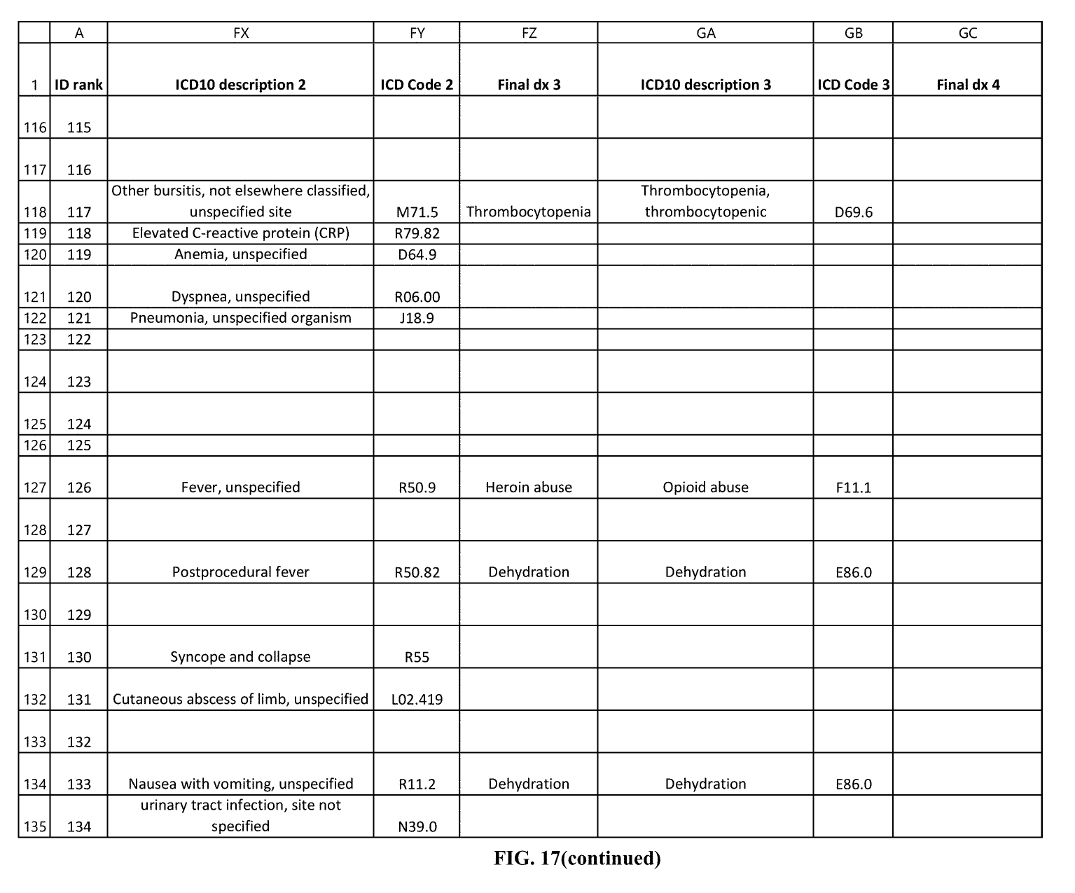

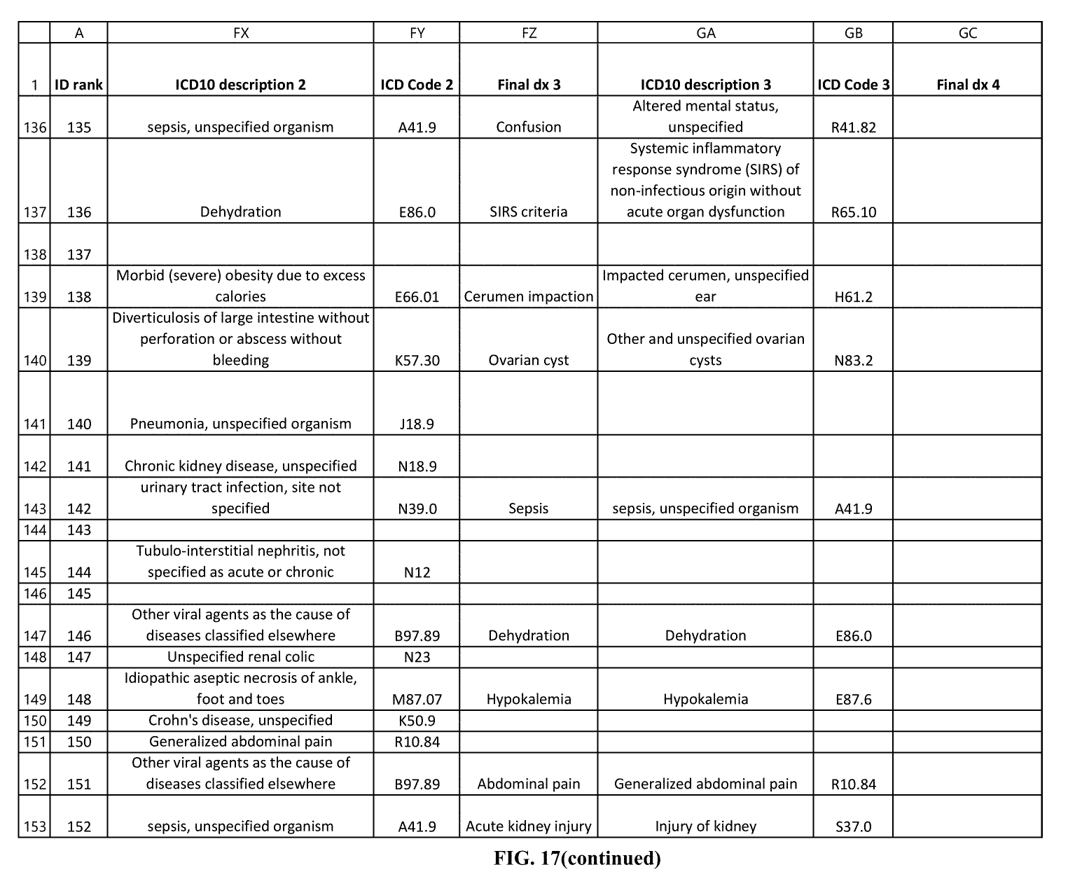

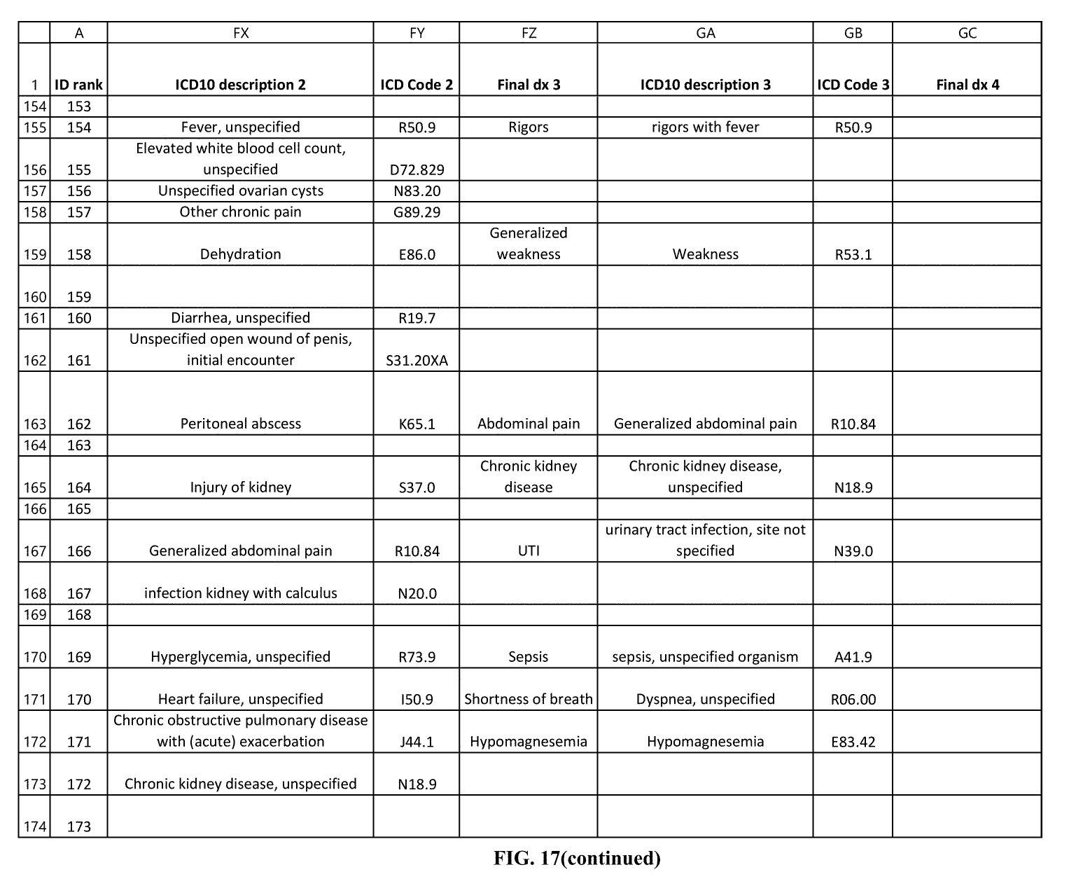

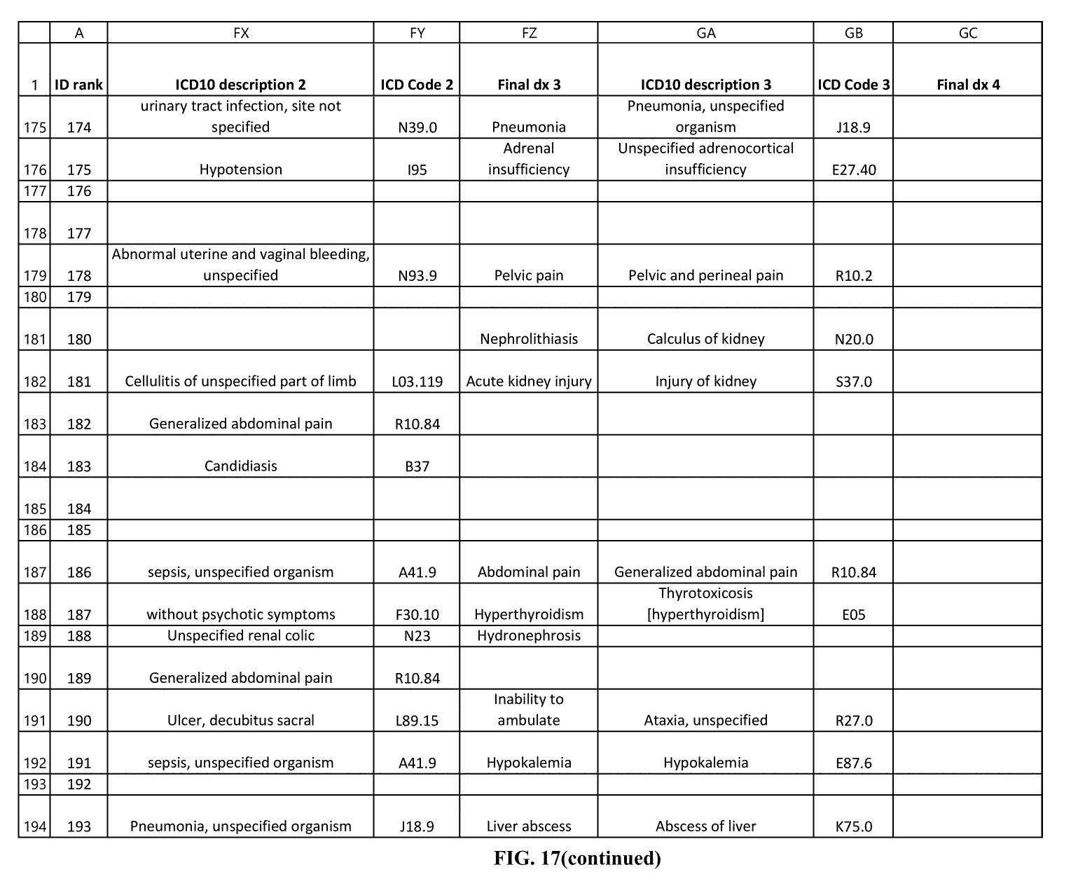

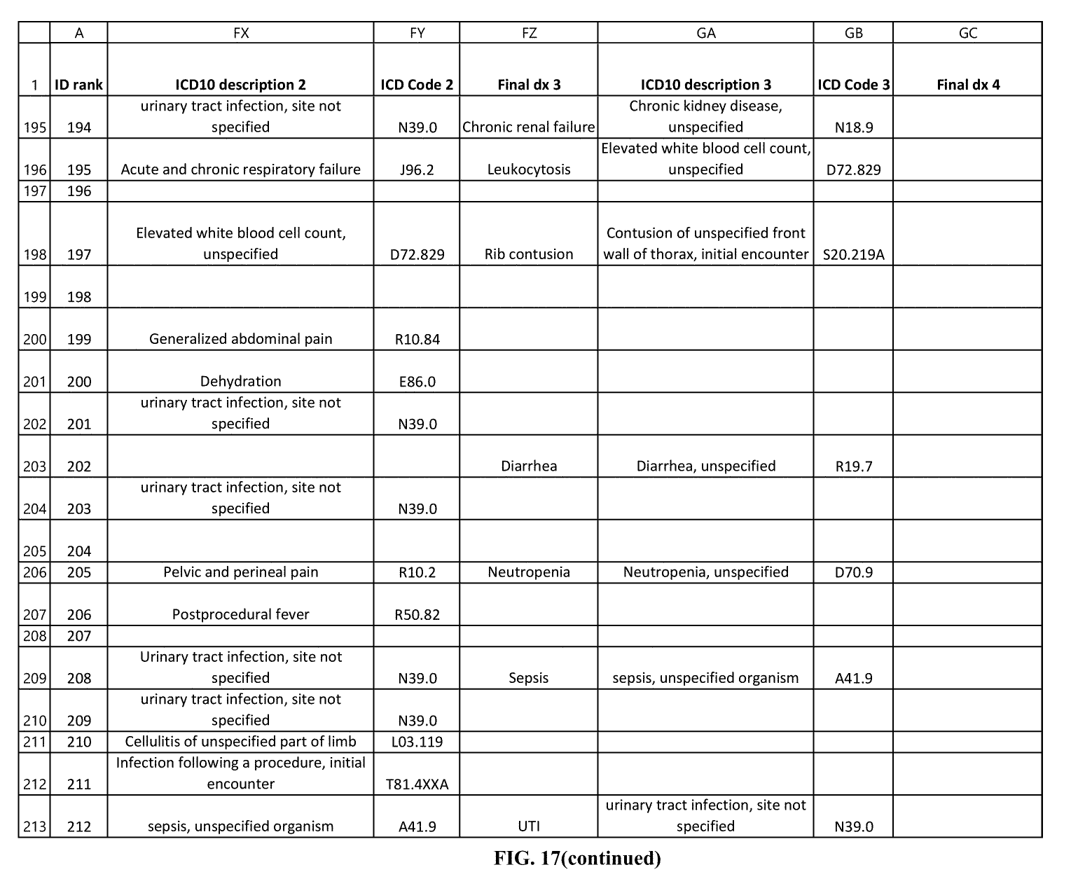

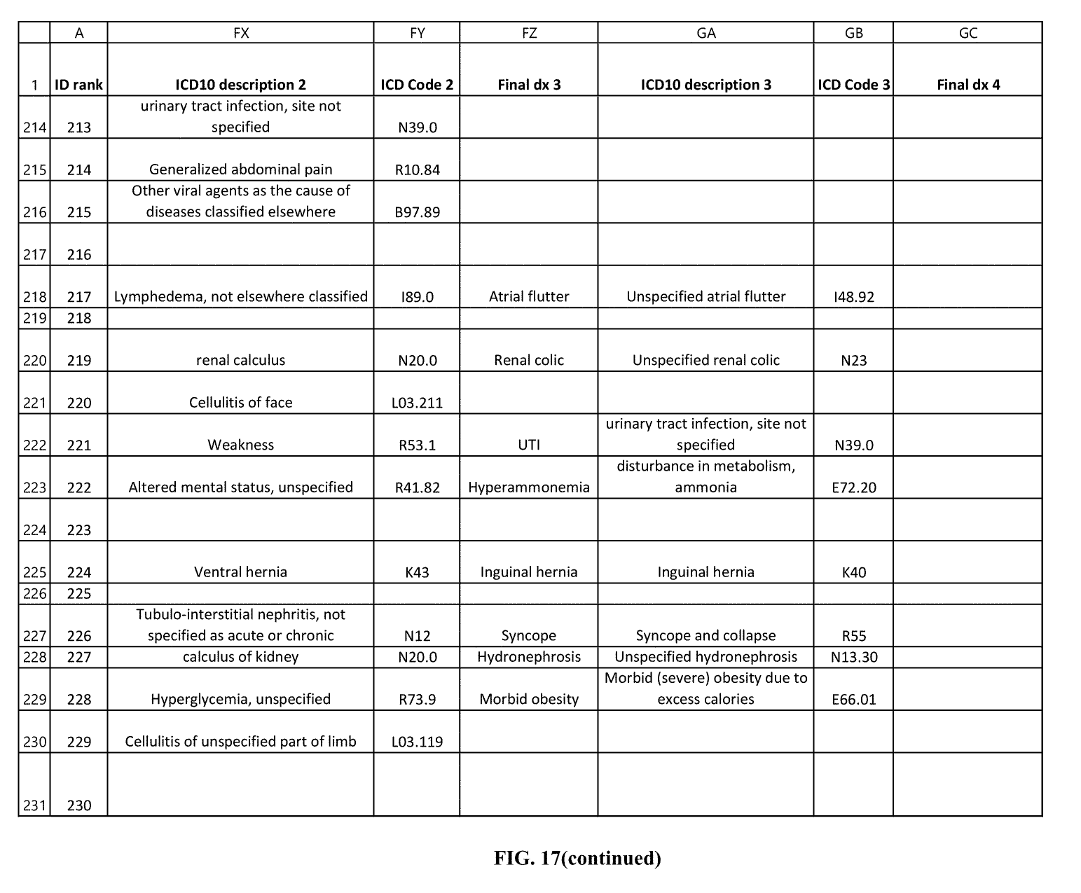

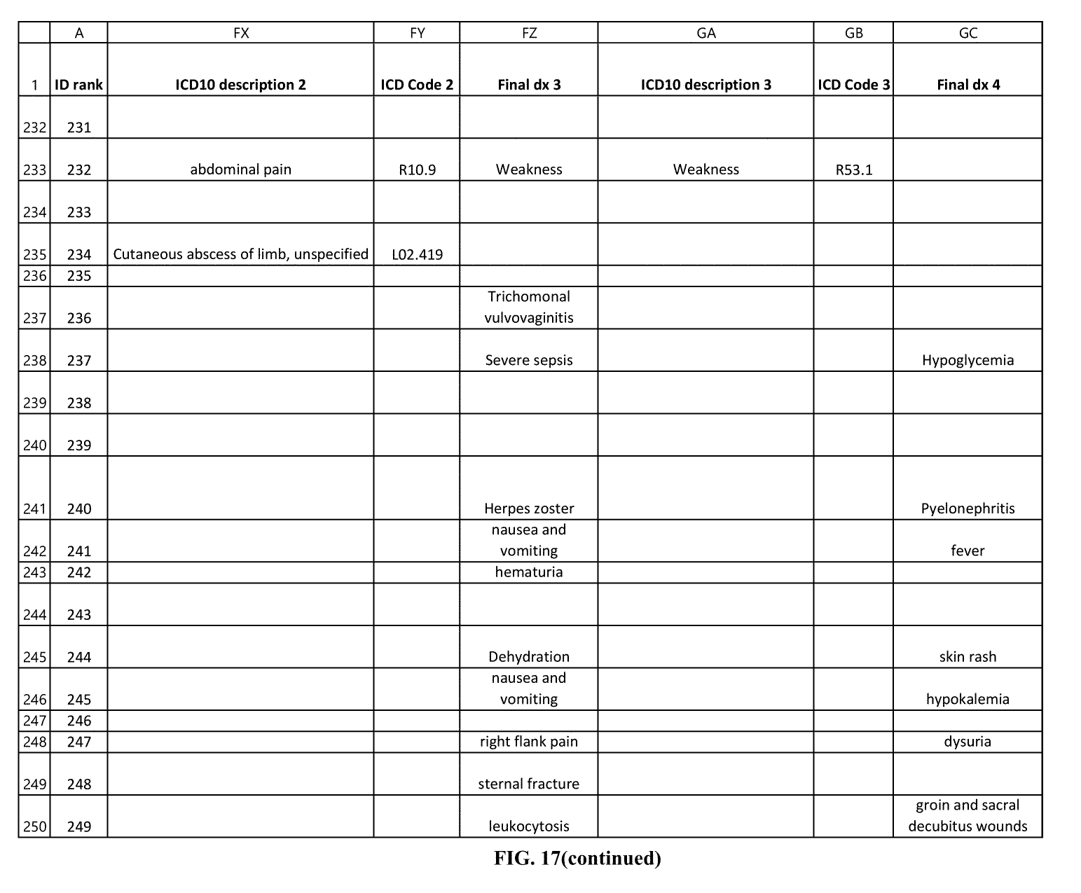

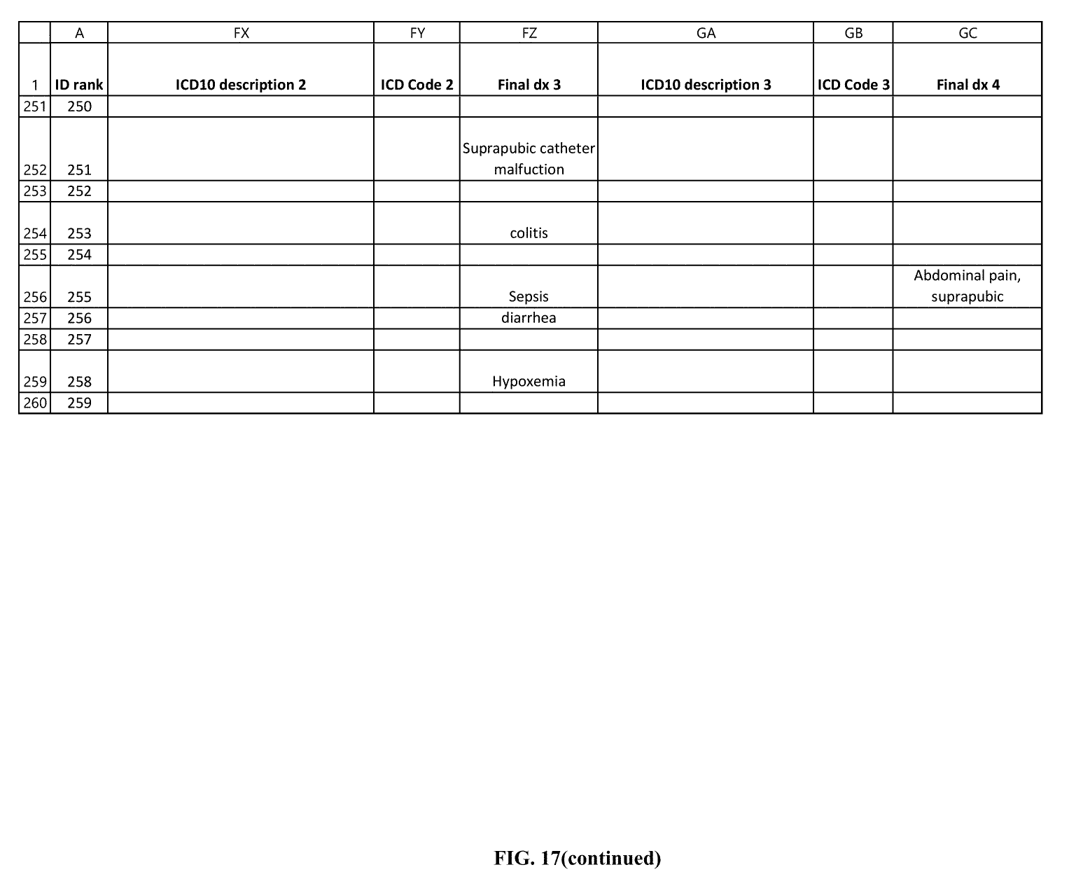

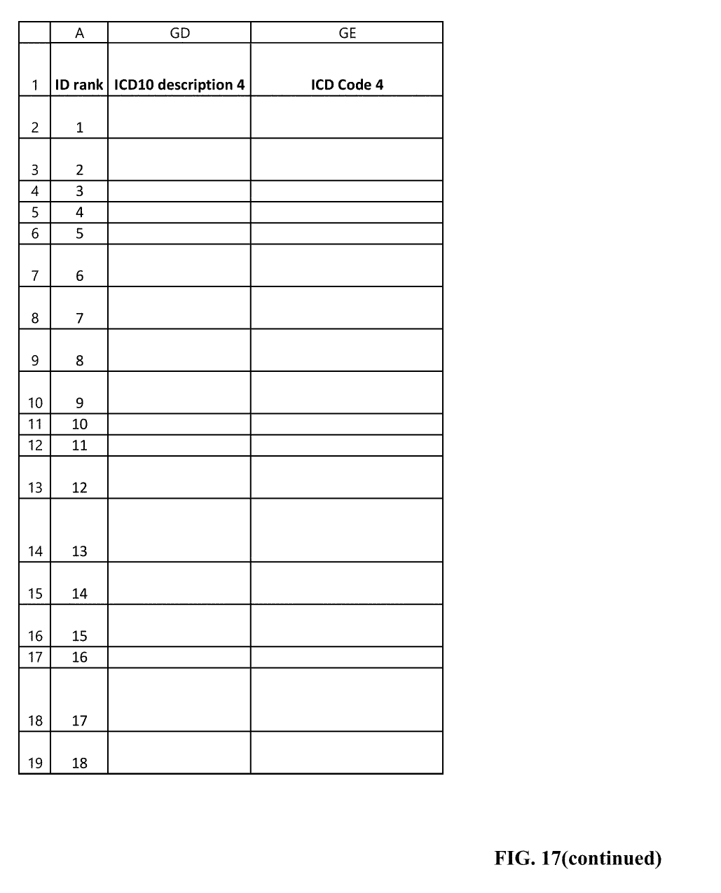

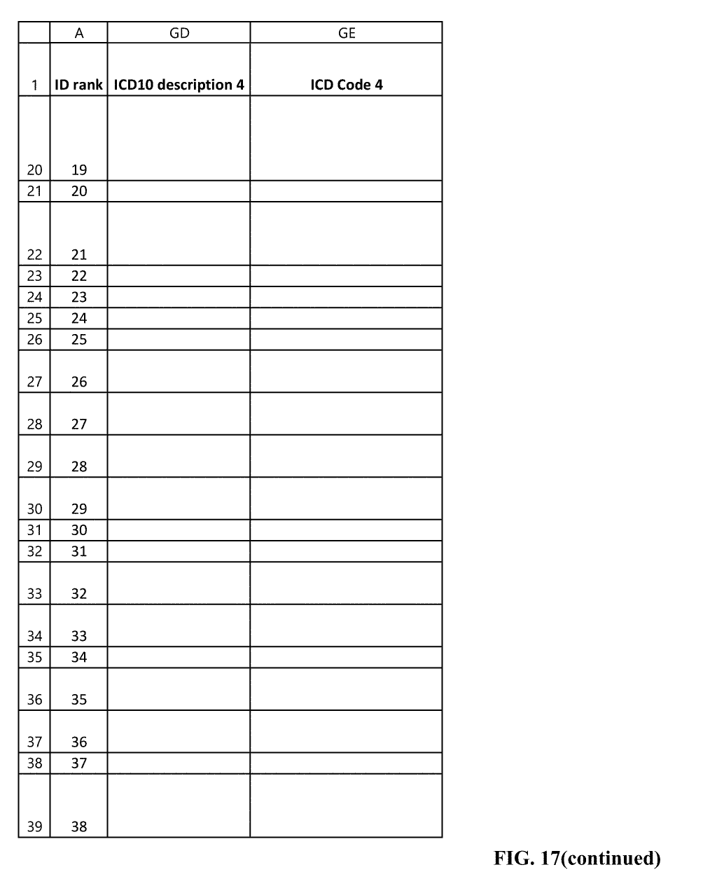

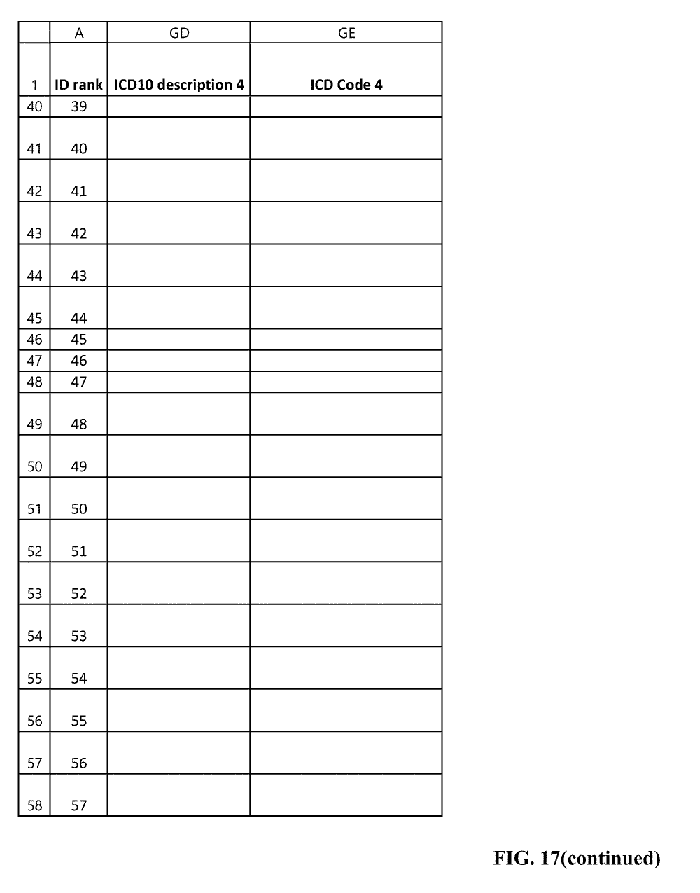

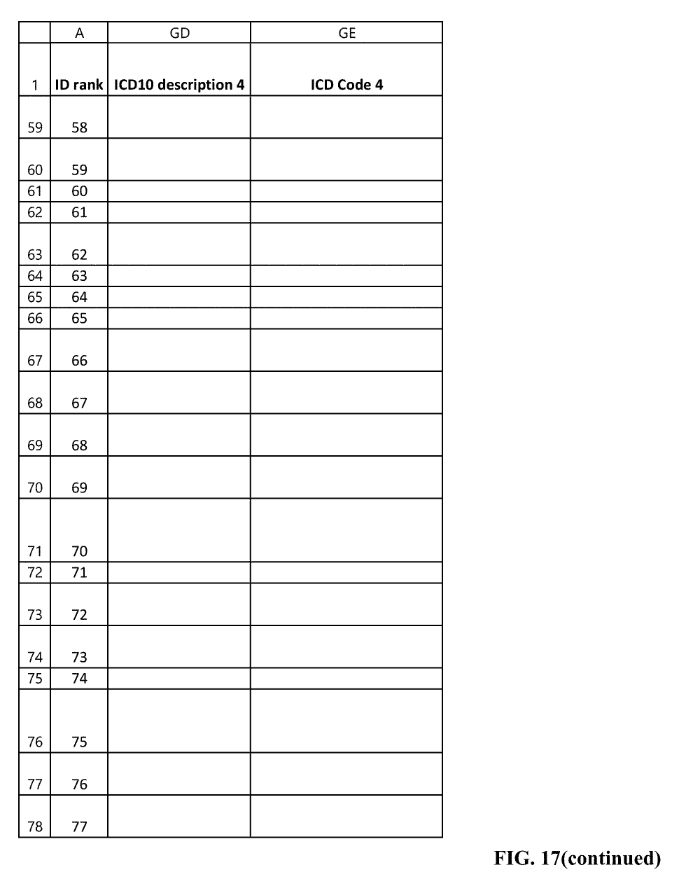

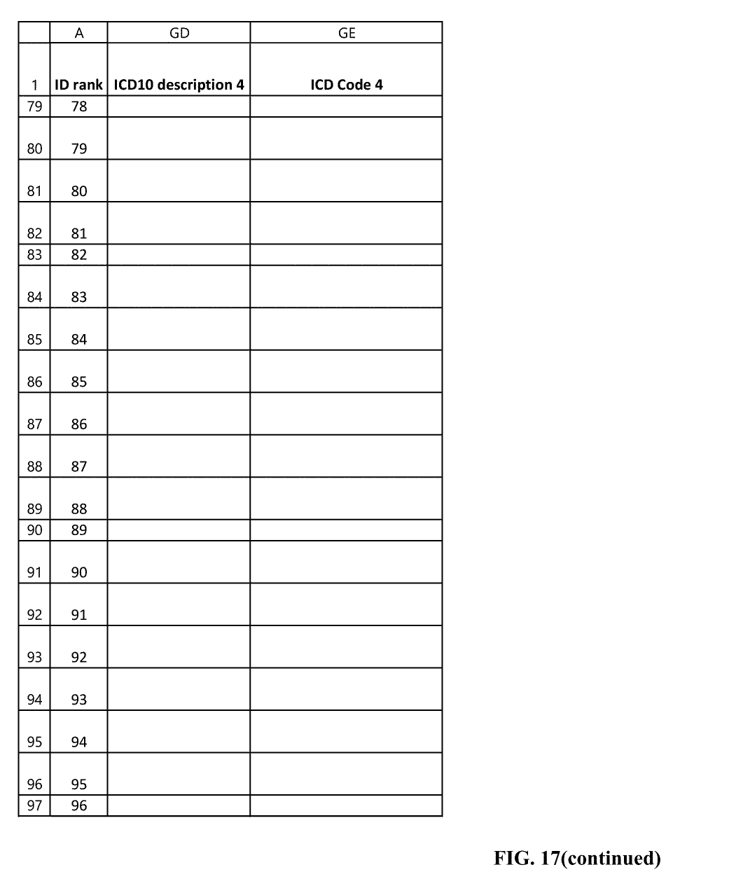

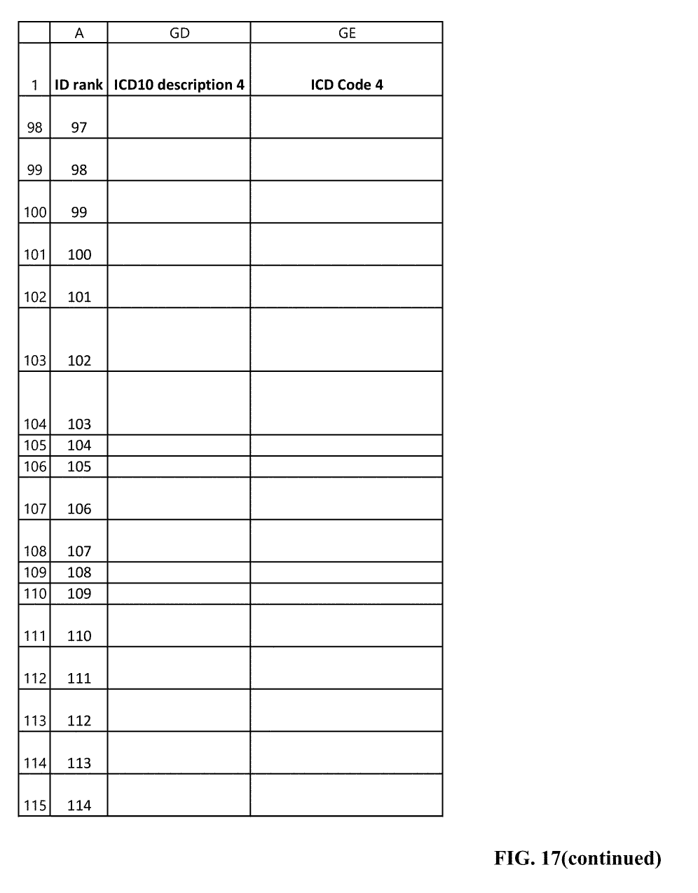

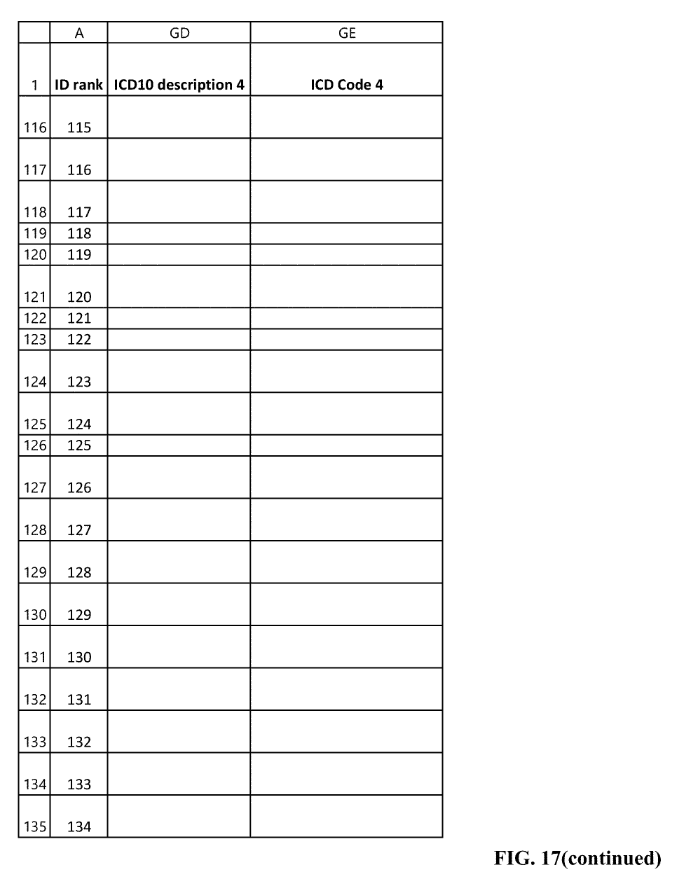

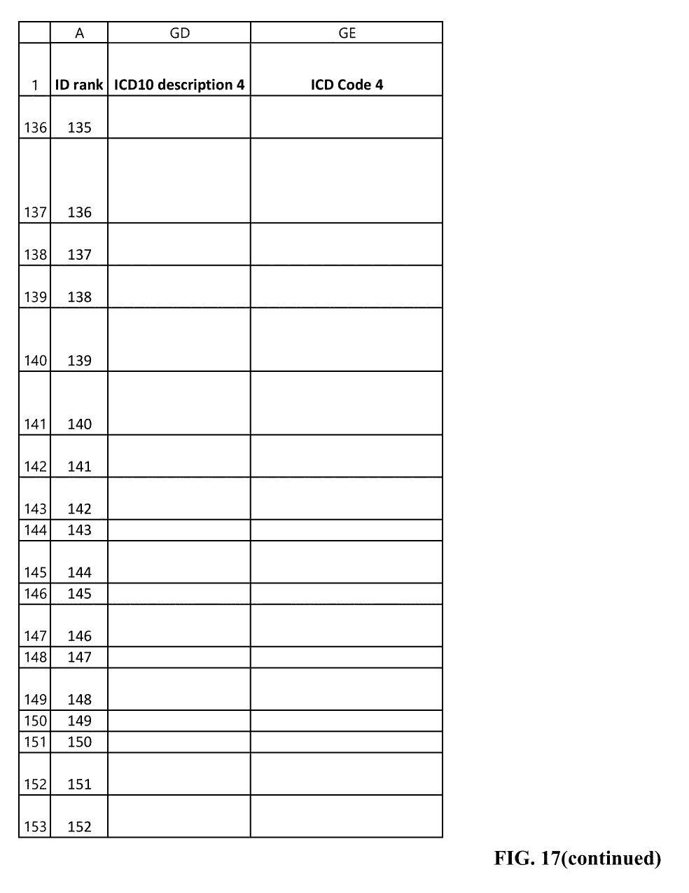

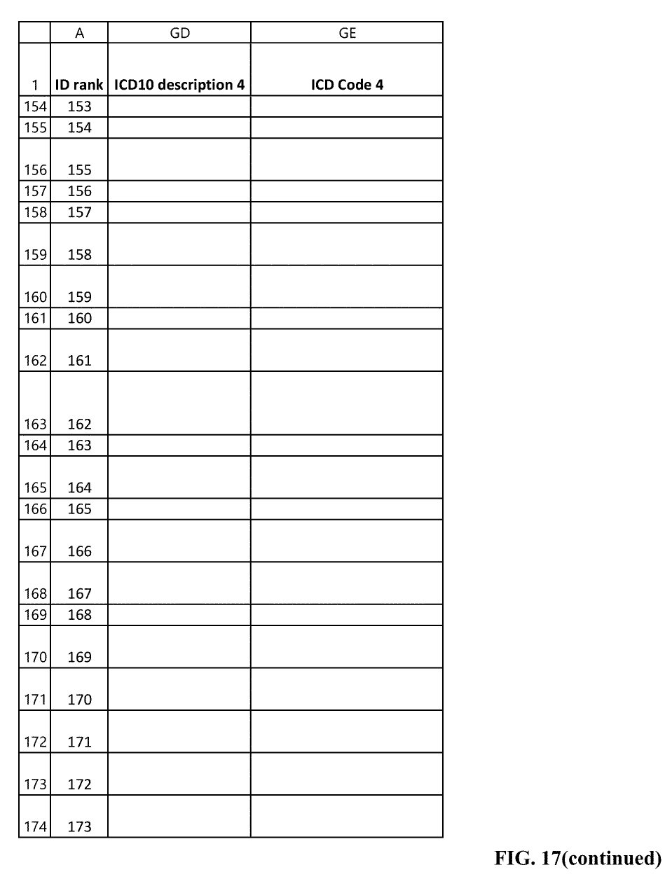

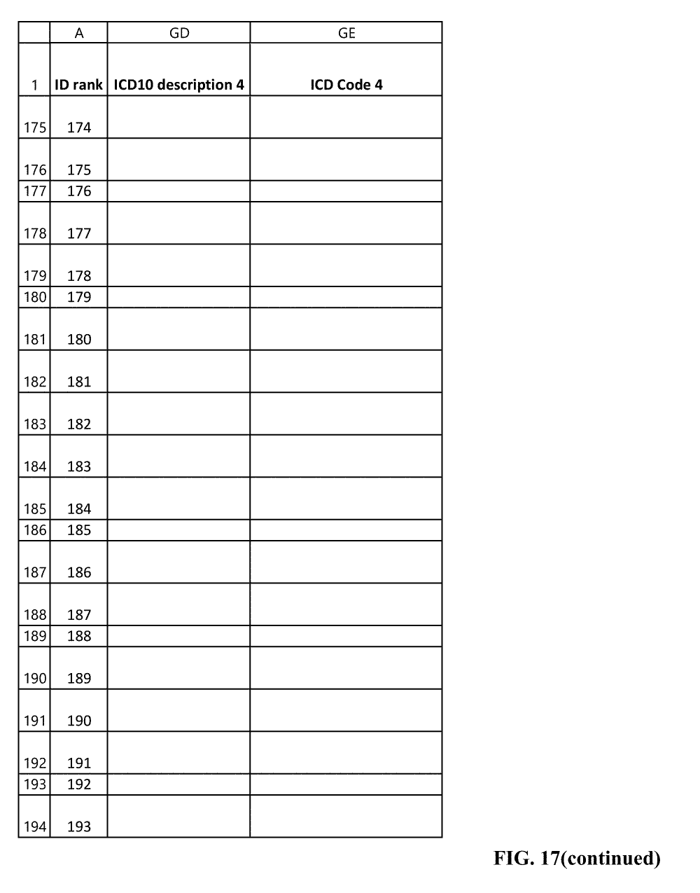

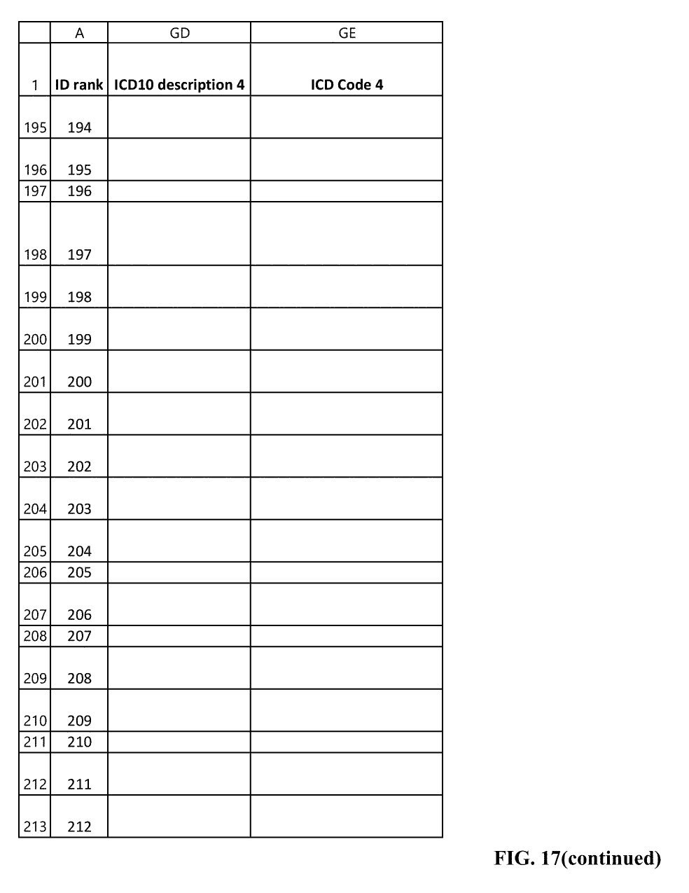

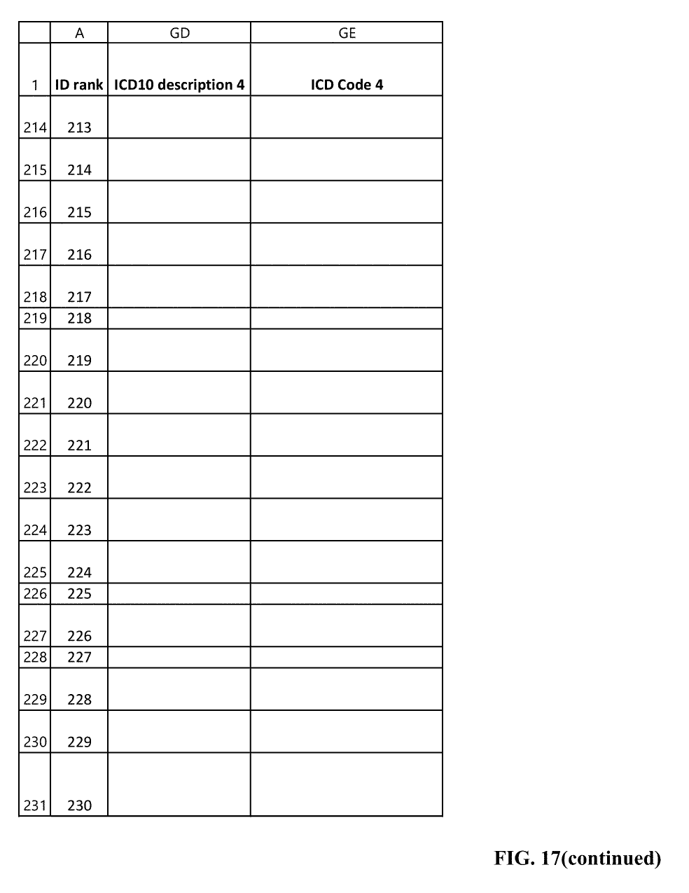

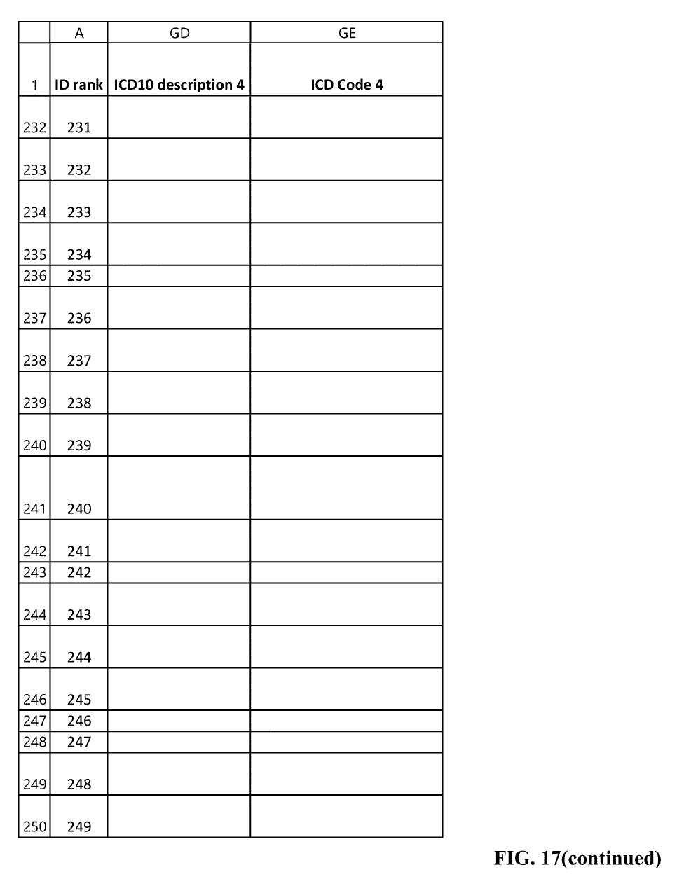

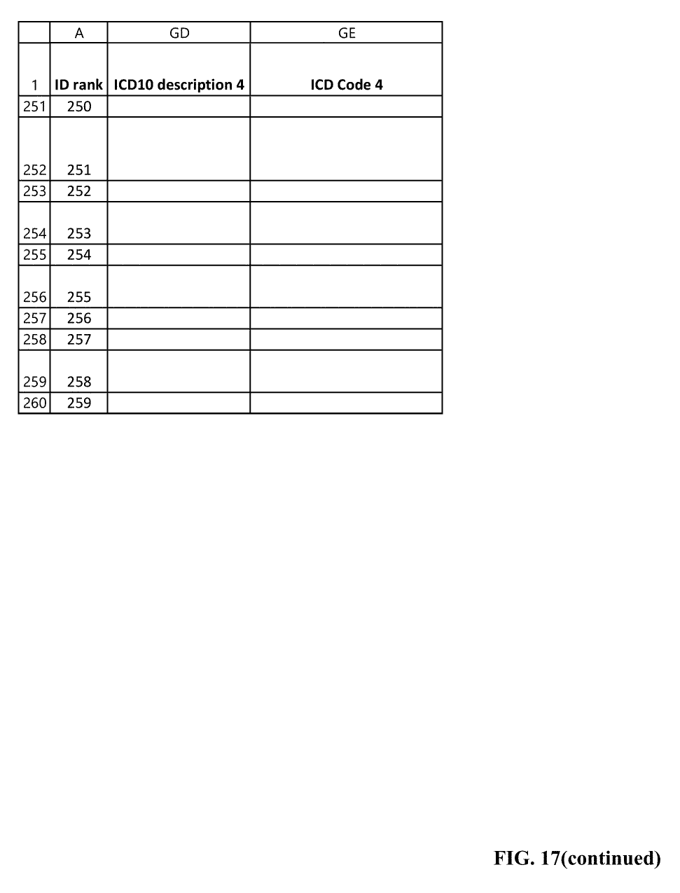

A total of 239 duplicate clinical blood (n=27), urine (n=122), wound/throat swab (n=73), expectorated sputum (n=16), stool (n=2) samples from 229 consecutive consenting Emergency Department patients with suspected infection were collected over a period of 16 months from McLaren of Greater Lansing Hospital and Sparrow Hospital. No cerebrospinal fluid samples met inclusion criteria for this study. The clinical samples used in this study were stored at 4° C. immediately after collection until nucleic acid template preparation. The stored samples were processed within 24 hours of clinical collection time. Clinical template extracts were applied to loop-mediated isothermal amplification (LAMP) reaction immediately following template preparation. Clinical samples were excluded in cases of suspected or confirmed external contamination and or other sample collection and processing problems. The clinical samples from patients with missing or erroneous consent forms were discarded (as shown in FIG. 17).

The preparation of the clinical fluid sample includes an initial processing time that was focused on the filtration of blood and urine through EconoSpin spin Column filter tubes (approximately 10 min) and the collection of samples from mucocutaneous swab tubes (approx. 3 min).

### Preparing the Clinical Fluid Sample for Heat Lysing:

In another embodiment, the LAMP assays for detection of 14 clinical pathogens as well as the mecA gene were compared with conventional hospital culture and PCR-based assays for sensitivity and specificity of detection directly from clinical samples obtained in the Emergency Department from consenting patients at two regional hospitals, Sparrow Hospital, a Level One Trauma Center, and McLaren of Greater Lansing Hospital, a Level Two Trauma Center, both in Lansing, Mich. The samples were first analyzed using LightCycler amplification and a subset were run using EBT-dye methods. The clinical samples with less than 6 mL of urine or an undetectable signal by LightCycler were excluded from EBT analysis.

### Clinical Sample: Blood

In one embodiment, the blood samples were prepared using 3 mL of blood from each Red-top BD Vacutainer Plus venous Blood collection Tube Serum Clot Activator, Purple-top BD Vacutainer K2 EDTA Venous Blood Collection Tube or BD BACTEC Plus Aerobic/F blood culture solution sample was taken and passed through an EconoSpin Column for crude DNA extraction. Then 100 μl of water was added to the column within a collection tube and heated on a heating block of from 90° C. to 110° C. for 10 to 20 min.

### Clinical Sample: Urine

In another embodiment, the urine samples were prepared using 3 mL of whole urine. Each clinical urine sample was passed through an EconoSpin Column for collection of genetic material. Then, 100 μl of water was resuspended on the column filter and heated on a heating block of from 90° C. to 110° C. for 10 to 20 min.

### Clinical Sample: Wound/Swab

In one embodiment, the would/swab samples were prepared by adding to the tube 100 μl of liquid was added and then aspirated from the bottom of a BD BBL CultureSwab EZ tube using 200 μl pipettes and transferred to a 1.5 mL Eppendorf tube. The repeated aspirations were to extract 100 μl from most tubes. The samples were then heated on a heating block of from 90° C. to 110° C. for 10 to 20 min.

### Clinical Sample: Stool and Sputum

In another embodiment, the stool and sputum samples were prepared by adding 400 IA of the sample directly from the clinical sterile hospital collection container to a 1.5 mL Eppendorf tube. The sample was then heated on a heating block of from 90° C. to 110° C. for 10 to 20 min.

### Heat Lysing the Clinical Fluid Sample to Form a Lysate:

After the preparation of the clinical fluid sample, the sample is heat lysed from 90° C. to 110° C. for 10 to 20 min. Lysis is the process by which a cell membrane is opened up to release its genetic material. Generally, lysing occurs by adding a chemical to the sample creating a mixture and heating the mixture.

Mixing the Lysate with a Target Primer, an Indicator, a Polymerase Enzyme, a Chemical Reaction Buffer in a Vial Forming a Reaction Mixture:

The reagent is an isothermal reaction reagent. The first mixture is pipetted onto 96-well plates preloaded with the target primers. A lysate is the fluid containing the contents of lysed cells.

In various embodiments, 8 μL of H₂O, 1 μl of 10× Isothermal Amplification buffer and 1 μl of sample or positive control were added to each well or PCR reaction tube.

In each reaction well the final reagents include 1× Isothermal Amplification Buffer; 6 mM MgSO₄; 1.4 mM of dATP; 1.4 mM of dGTP; 1.4 mM of dCTP; 1.4 mM of dTTP; 1 μg/ul of BSA; 0.4% of Pluronic F-68; 0.284% Glycerol; 0.16 M trehalose; 1.6 μM FIP primer; 1.6 μM BIP primer; 0.8 μM LF primer; 0.8 μM LB primer; 0.2 μM F3 primer; 0.2 uM B3 primer; 75 μM EBT or 20 μM Syto 82; 1 μl of template or positive control.

The mixture, a target DNA primer or a target RNA primer, an indicator, a polymerase enzyme, and a chemical reaction buffer is loaded into the vial to form the reaction mixture.

In various embodiments, the LAMP primers were pre-dispensed in 96-well plates to result in a final concentration of 1.6 μM FIP primer, 1.6 μM BIP primer, 0.8 μM LF primer, 0.8 μM LB primer, 0.2 μM F3 primer and 0.2 μM B3 primer, including one target assay per well or vial. In another embodiment, the indicator is EBT Dye or Syto-82 Dye. In yet another embodiment, the reaction buffer includes 10× Isothermal Amplification Buffer (New England Biolabs), Pluronic F68 and magnesium sulfate.

In various embodiments, a subset of positive clinical samples identified by the LightCycler method was tested in 96-well PCR plate format using the colorimetric color indicator azo dye Eriochrome Black (n=12). EBT is an indicator that causes a color change of solution according to calcium or magnesium concentration. As Mg²⁺ concentration decreases during a positive LAMP reaction in the presence of EBT, the solution changes from purple to blue. In one embodiment, EBT dye is a complexometric indicator that is used in complexometric titrations. In addition, EBT dye is an azo dye. Azo dyes are organic compounds. The color change measurements were identified visually and using a spectrophotometer (Genesys 10 UV) with measurements recorded at 5 minute intervals from 0 to 45 minutes at 380 nm, 400 nm, 420 nm, and 475 nm wavelengths.

Once the reaction mixture is mixed and loaded into the vial (well or PCR tube), the vial is then sealed and loaded onto the well plates.

### Incubating the Reaction Mixture:

In one embodiment, the data related to incubating the reaction mixture applies to the methods 300 and 500.

In another embodiment, limitations include a longer incubation time to detect low abundance targets (such as resistance genes including mecA) when the clinical sample volume is limited. The reaction mixture was incubated from 60° C. to 70° C. for 30 to 40 minutes on a 96-well block heater (Thermo Scientific Compact Digital Dry Bath/Block Heater).

In one embodiment, the incubating of the vial containing the reaction mixture placed in an incubator. The incubator maintains the optimal temperature, humidity, and other conditions.

### Amplifying the Reaction Mixture Using LAMP:

In another embodiment, the data related to amplifying the reaction mixture using loop mediated isothermal amplification applies to the methods 300, 400, and 500.

The step of amplifying the reaction mixture using LAMP is maintained at a temperature from 55° C. to 67° C. for 30 to 40 minutes. Any steps known in the art are used for LAMP amplification. The step is performed with a heating block capable of reaching isothermal amplification temperatures.

Loop mediated isothermal amplification (LAMP) is a single tube technique for the amplification of DNA or RNA. LAMP is carried out at a constant temperature from 55° C. to 67° C. for 30 to 40 minutes. The target sequence is amplified at the constant temperature using either two or three sets of primers (discussed above). Typically, 4 different primers are used to identify 6 distinct regions on the target gene, which adds highly to the specificity.

In one embodiment, EBT-based color change reactions offer the strengths of detection by direct amplification without the potential limitations present with advanced electronics utilized with a thermocycler-based platform. A longer amplification time from 40 to 45 minutes is used on a separate plate to identify lower abundance genes. In yet another embodiment, to prevent nonspecific amplification from species-specific metabolic genes and increase chances of false positive identification of the targets running concurrently amplify in the same timeframe for naked eye detection of color change. When samples are pre-cultured for increased target abundance, such template concentration-restricted scarcity are overcome.

In various embodiments, after heating and amplifying 1 μl of template from each clinical sample type was added per 10 μl reaction well in a 96-well plate format and analyzed on by LAMP Thermocycler and/or Eriochrome Black T (EBT) colorimetric change.

### Cooling the Reaction Mixture Thereby Stopping the Amplification:

In yet another embodiment, the data related to immersing the reaction mixture in an ice bath to stop the amplification applies to the methods 300 and 500. In addition, the amplification of the reaction mixture can be stopped by placing the reaction mixture in a refrigerator or a freezer for a predetermined amount of time.

The temperature of the ice bath is from 8° C. to 15° C. The predetermined amount of time is from 30 second to a minute and 30 seconds. The immersion is performed with an ice bath.

In one embodiment, a cooling bath or an ice bath is a liquid mixture which is used to maintain a low temperature. The low temperature are used to perform a chemical reaction below room temperature, e.g. EBT colorimetric change.

The LAMP reaction mixture was incubated at 63° C. for 40 minutes on a 96-well block heater (Thermo Scientific Compact Digital Dry Bath/Block Heater) followed by immediate immersion in an ice water bath (to stop the amplification and decrease condensation on clear sealing tape) for one minute before immediate visual color change discrimination from purple to blue by one or more non-color blind human examiners. The colorimetric detection results remained stable for at least 24 hours.

### Identifying a Color Change in the Reaction Mixture:

In various embodiments, the data related to identifying a color change in the reaction mixture applies to the methods 300 and 500.

The identification of a color change is performed with an individual who is able to detect color change from purple to blue.

In another embodiment, the EBT color measurement was performed by a spectrophotometer at the wavelengths 380 nm, 400 nm, 420 nm and 475 nm. In another embodiment, the 70 μL LAMP amplification systems were employed for this purpose. After the LAMP amplification, 64 μL of the reaction mixture was taken to the test cuvettes which were preload with 448 μL of water (8 times dilution). After mixing well, absorbance values of the cuvettes were read by a GENESYS 10 Series spectrophotometers from Spectronic Unicam.

In another embodiment, the EBT dye analysis includes spectrophotometric data recorded by readings for positive and negative samples at 5 minute intervals from 0 to 45 minutes at wavelengths 380 nm, 400 nm, 420 nm and 475 nm. The amplification of at least two of the triplicate wells per sample was for the determination of positive. The cutoff of 45 cycles was used to minimize false-positive and nonspecific primer amplification (e.g. shown in FIGS. 16(a) and 16(b)).

In another embodiment, identical LAMP primers used for the LightCycler method (1.6 μM FIP primer, 1.6 μM BIP primer, 0.8 μM LF primer, 0.8 μM LB primer, 0.2 μM F3 primer and 0.2 μM B3 primer) were pre-dispensed into 96-well plates. Each 10 μL of the reaction mixture contained 10× Isothermal Amplification Buffer (New England BioLabs), 6 mM magnesium sulfate, 0.64 Unit/μl Bst 2.0 DNA polymerase (New England BioLabs), Eriochrome Black 75 μM (Sigma Aldrich), 0.4% Pluronic F-68, 1 μg/μl BSA, 350 μM of each dNTP, and 1 μl Template. Pluronic F-68 is a non-ionic surfactant used to control shear forces in suspension cultures. One target assay was included per well.

In various embodiments, the threshold for identification of a positive amplification was positive color change from purple to blue in at least two out of three wells for each gene target. The results were determined by visual discrimination by one or more non-color blind observers who were blinded to the targets in each well. The of the plates were read immediately after the amplification was compled.

As with real-time cycler analysis, each target was tested triplicate with one positive control and one negative no-template control (5 wells per target). The targets 1 to 14 were always included in clinical urine, mucocutaneous swab and stool analysis, where C. difficile was the target for clinical stool samples (n=1).

### Analyzing the Reaction Mixture by Thermocycler:

In various embodiments, the data related to analyzing the reaction mixture by thermocycler applies to the methods 400 and 500.

In various embodiments, after heating 1 μl of the clinical fluid sample, each clinical sample type was added per 10 μl reaction well in a 96-well plate format and analyzed on by LAMP Thermocycler.

The LAMP Thermocycler reactions were carried out on Roche LightCycler 96 System in 10 μl volume. The LAMP reaction mixture contained 1× Isothermal Amplification Buffer (New England BioLabs), 6 mM magnesium sulfate, 0.64 Unit/μl Bst 2.0 DNA polymerase (New England BioLabs), 20 μM Syto 82 (Molecular probes/Life Technologies), 0.4% Pluronic F-68, 1 μg/μl Bovine Serum Albumin (BSA), 350 μM of each dNTP (Invitrogen), and 1 μl Template. The Bovine Serum Albumin is a serum albumin protein or a globular protein derived from cows that is used in biochemical applications due to its stability and lack of interference within biological reactions.

In another embodiment, the primers were synthesized by Integrated DNA Technologies, Inc. The LAMP LightCycler reactions proceeded at 63° C. for 40 minutes. Each target was tested in triplicate with one positive control and one no-template control (5 wells per target). The targets 1 to 14 were always included in clinical blood, urine, mucocutaneous swab, and sputum analysis, where C. difficile was the target for clinical stool samples (n=2).

In one embodiment, for real-time LAMP analysis, the cycles to threshold (Cₜ) was calculated from the starting point of the amplification signal curve increased 0.01 (arbitrary units) above the baseline signal calculated by LightCycler default auto analysis. The amplification of at least two of the triplicate wells per target was for determination of a sample as positive. The latest of the triplicate positives for each sample was recorded as the amplification time for this sample. A cutoff of 40 cycles (53 seconds per cycle) was used to minimize false-positive and nonspecific primer amplification. The exception was for C. difficile primers, which were found to amplify later. The cutoff for the C. difficile primer set was set to 40 min (a shown in FIG. 13).

In various embodiments, the threshold cycle (Cₜ) was calculated as time in which the amplification signal increased 0.01 arbitrary units above the original signal for the real-time analysis. Moreover, one cycle is completed every 53 seconds. Overall, results were considered positive if two out of three wells exhibited amplification.

In another embodiment, the thermocycler Syto-82 method is completed of from 60 to 120 minutes.

### EBT Dye Method 300 and 500 Example:

In various embodiments, the EBT-LAMP reactions were completed within 120 min from start to finish per clinical sample. The time savings was gained when multiple samples were processed simultaneously in batches of up to three 96-well plates. In various embodiments, described herein, the clinical samples were prepared based on the type of clinical sample being tested. Once the clinical samples were prepared for the method 300, the clinical samples were added to the pre-prepared tubes (described above).

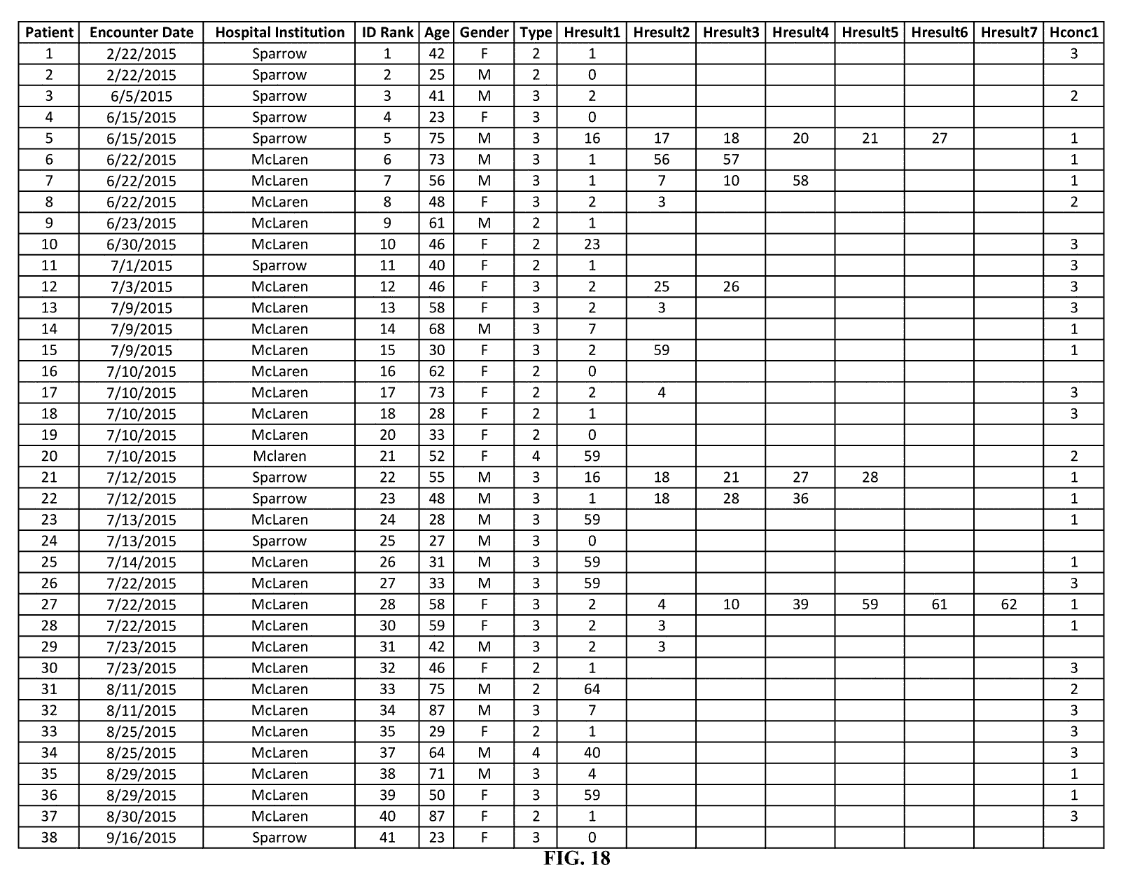

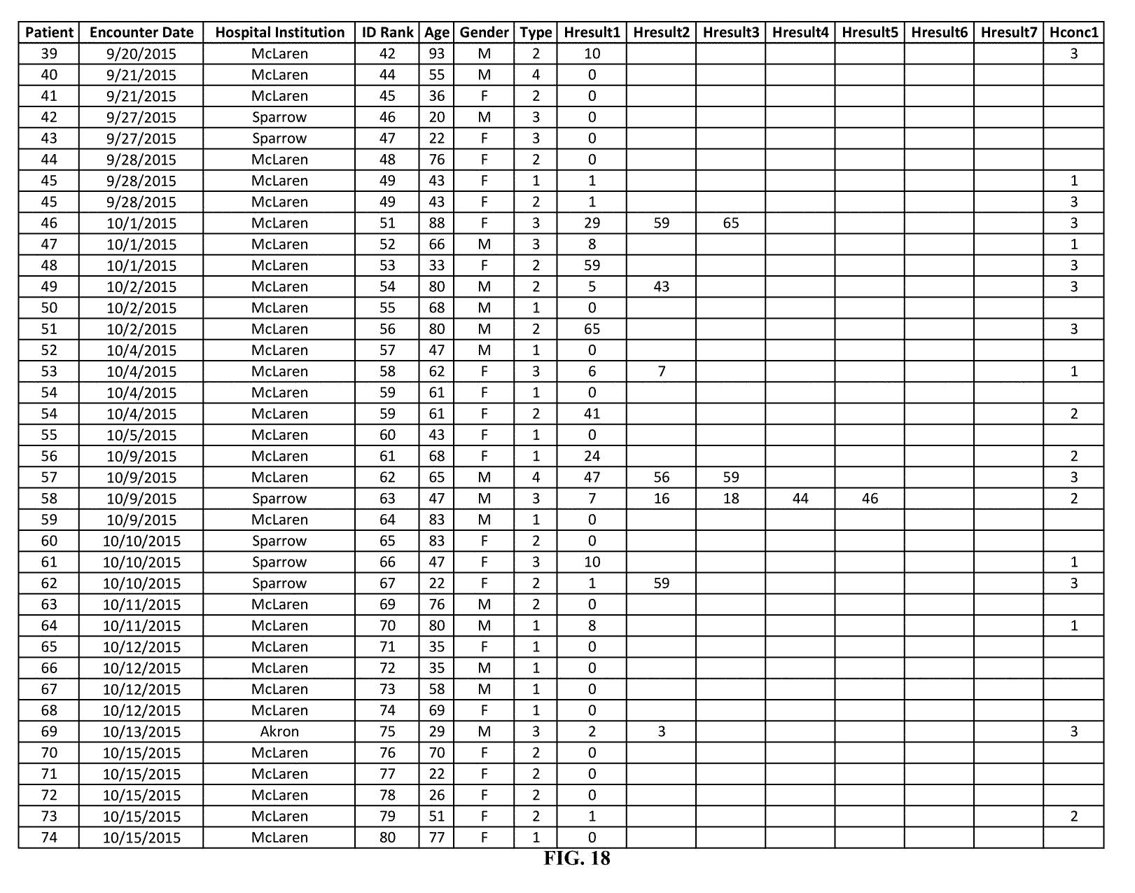

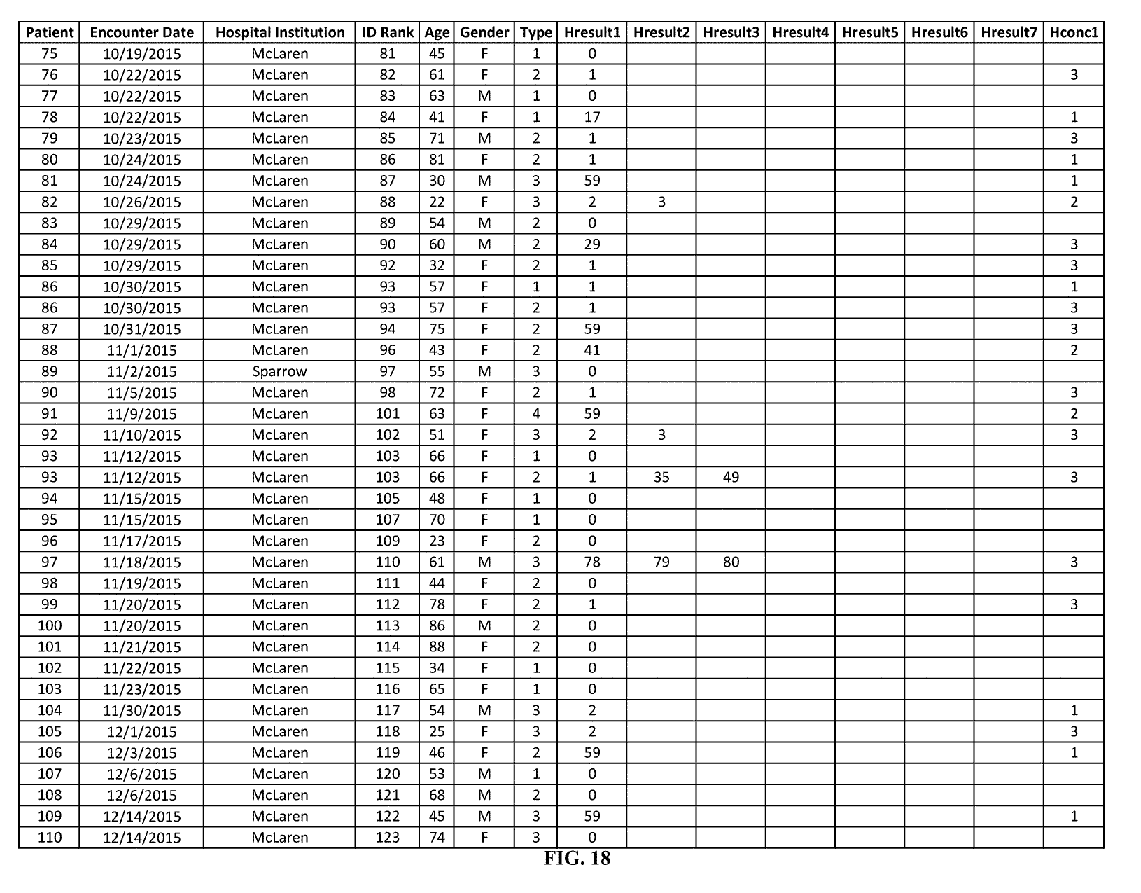

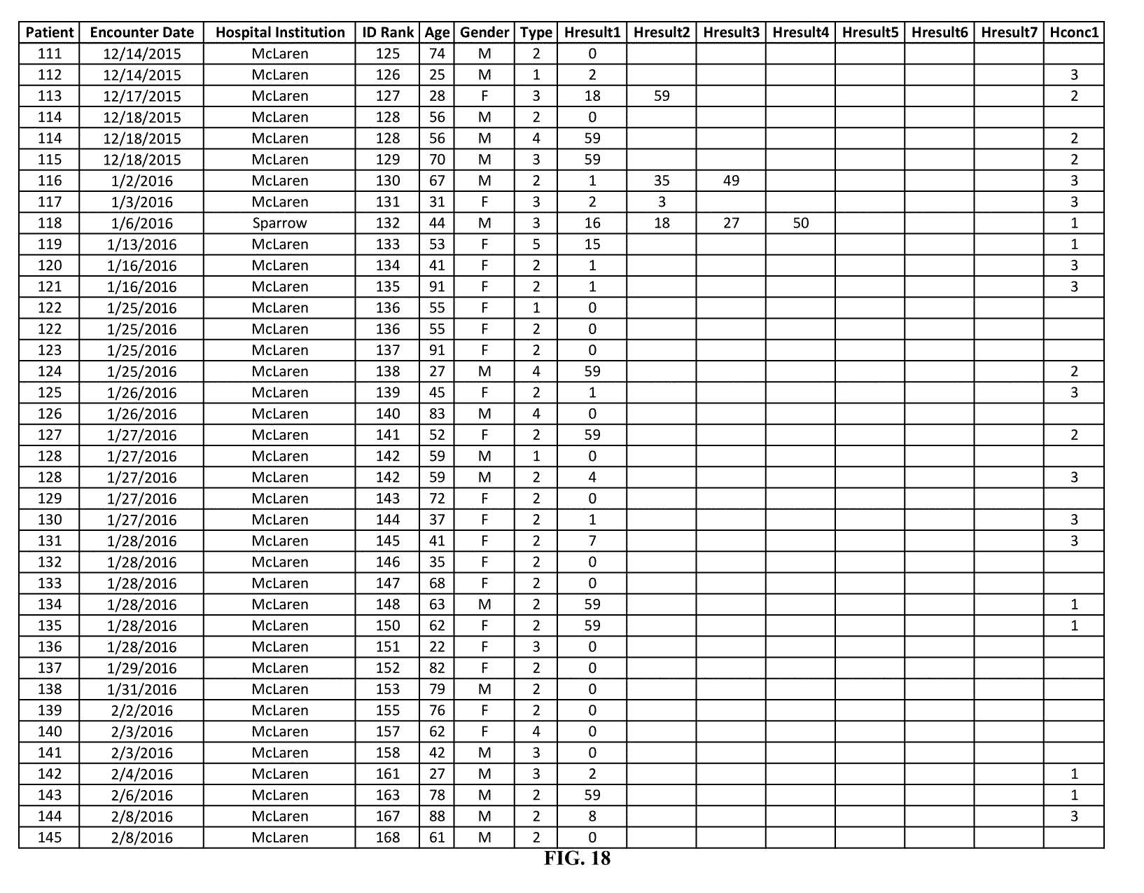

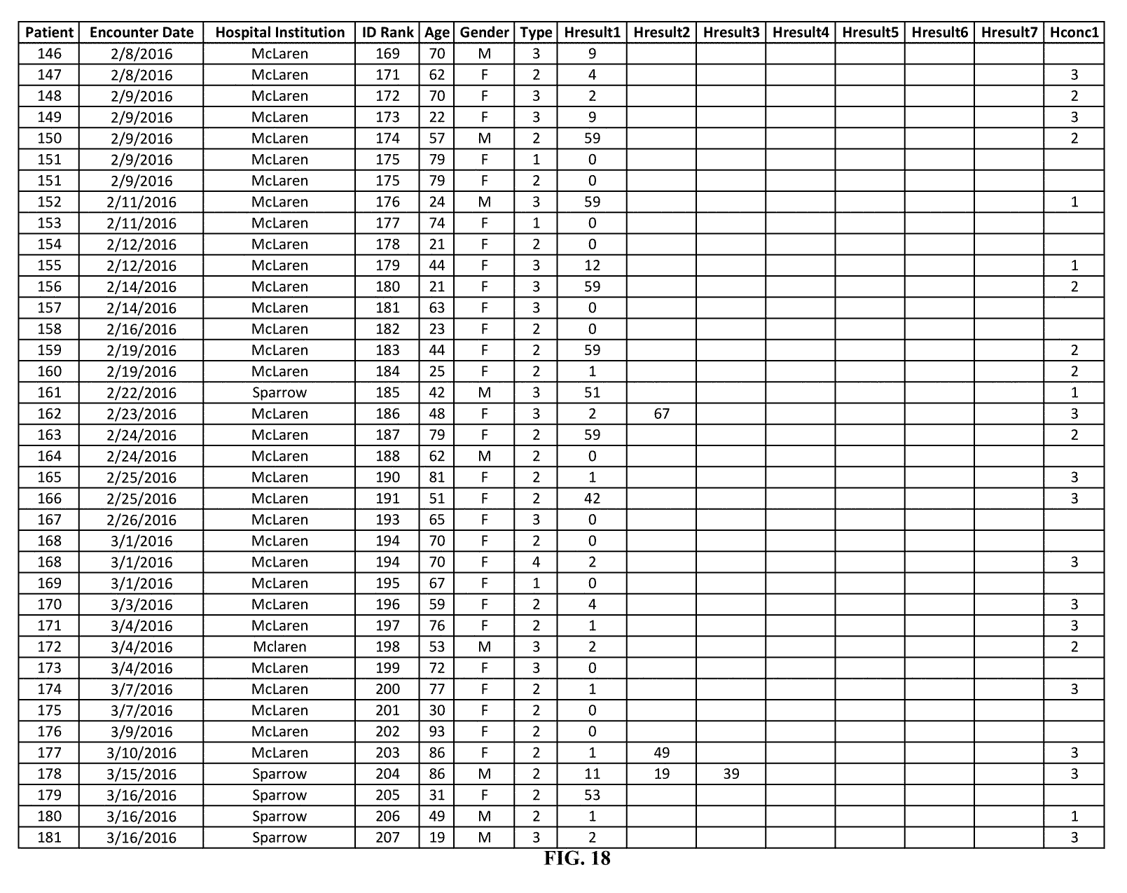

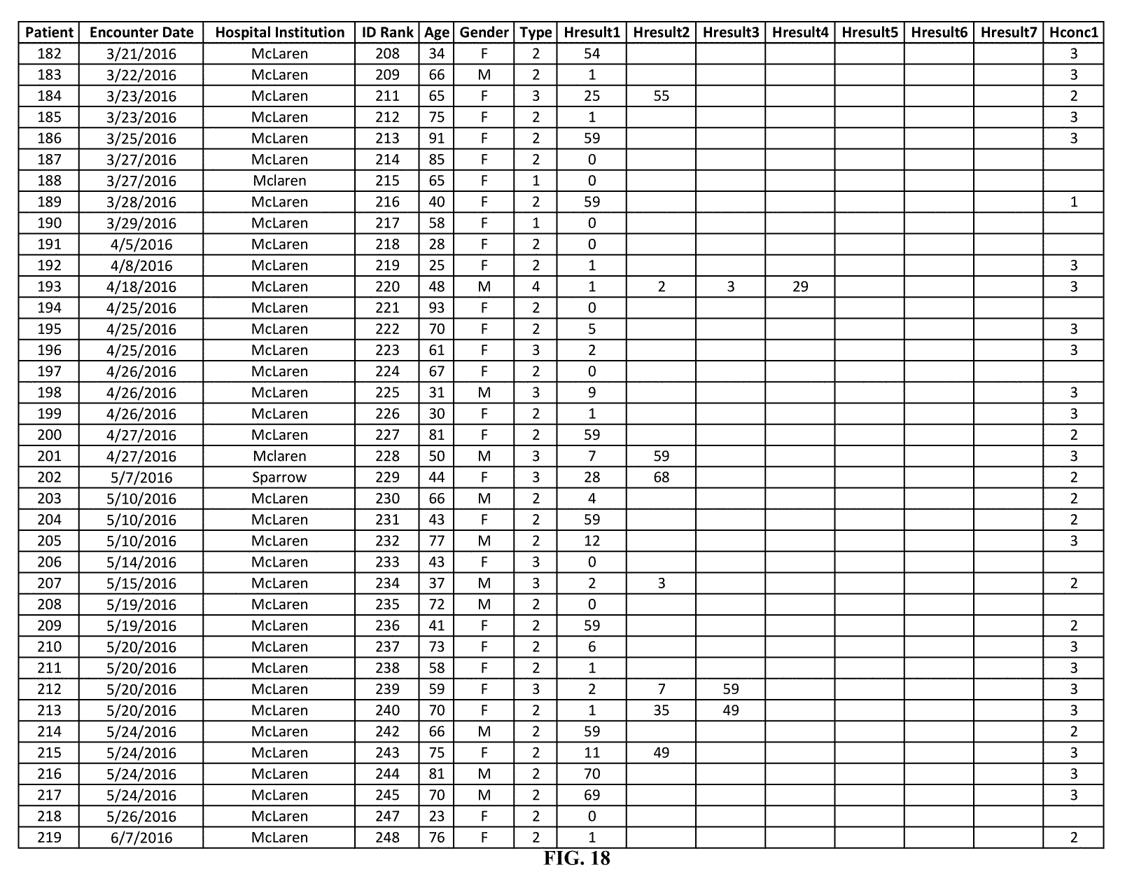

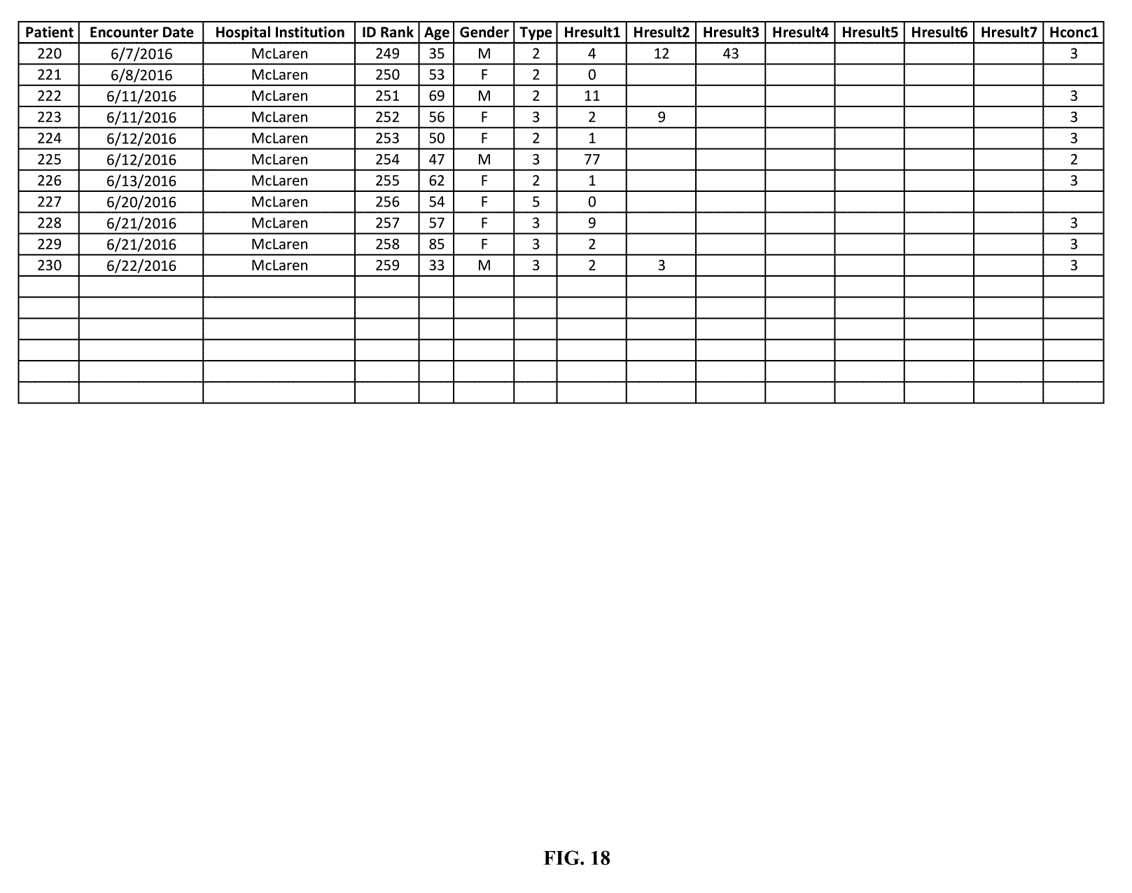

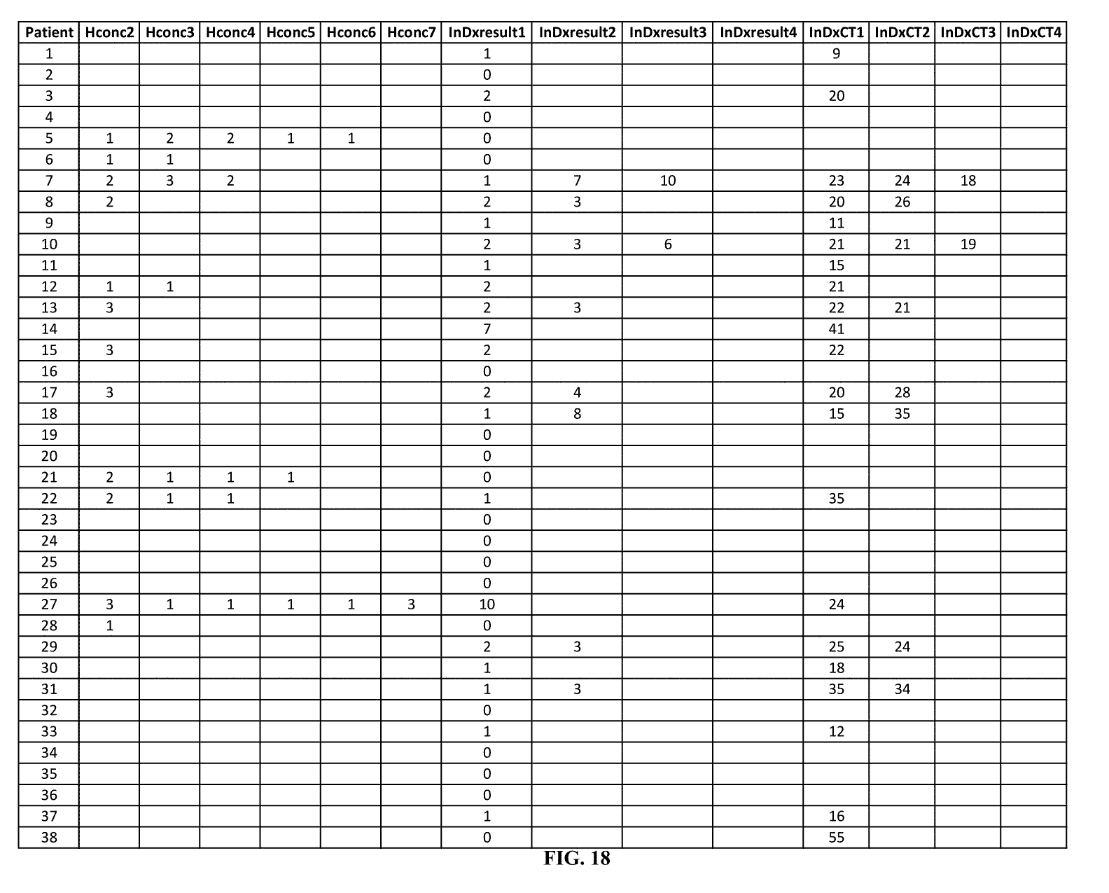

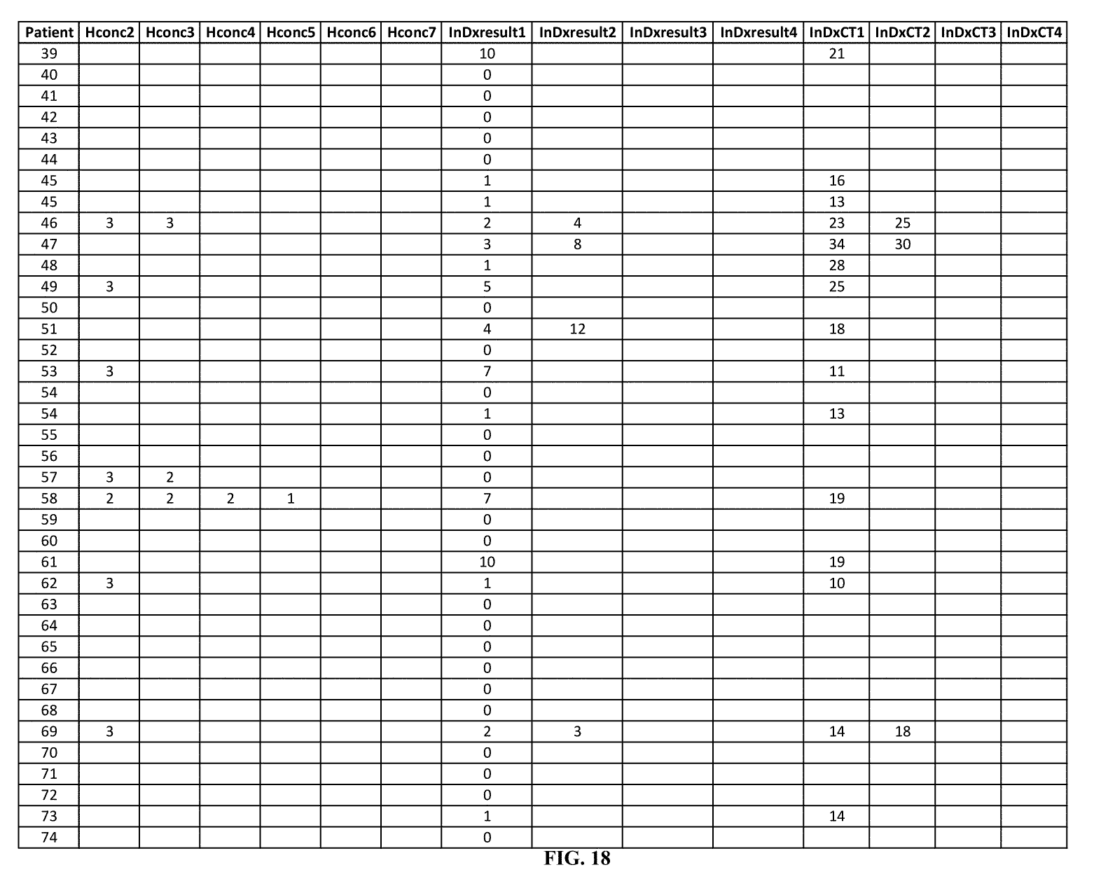

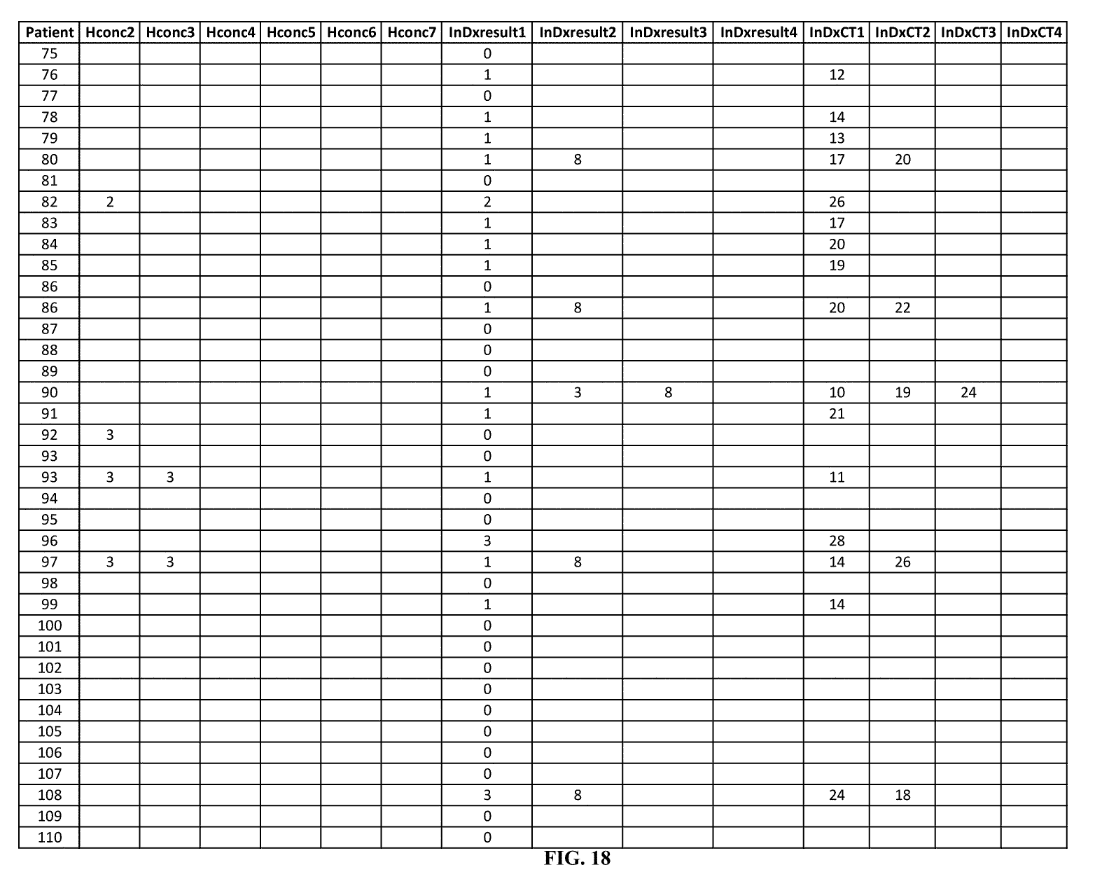

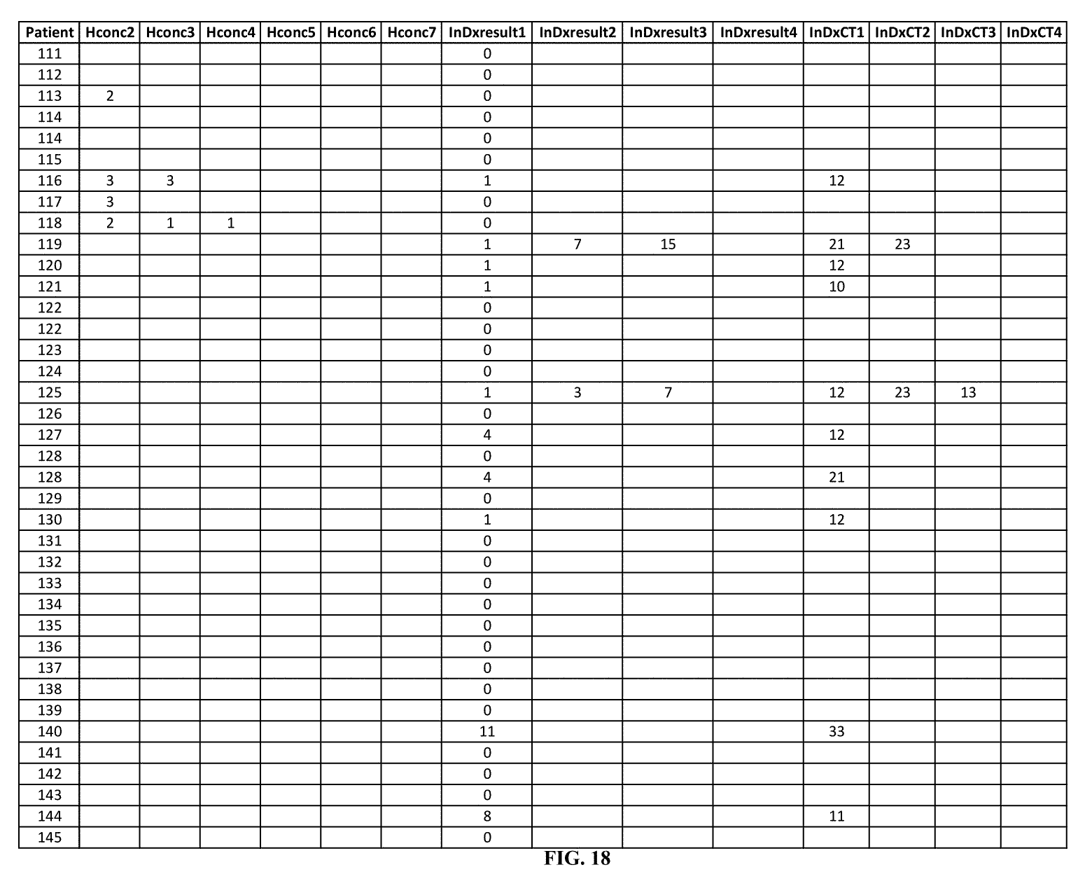

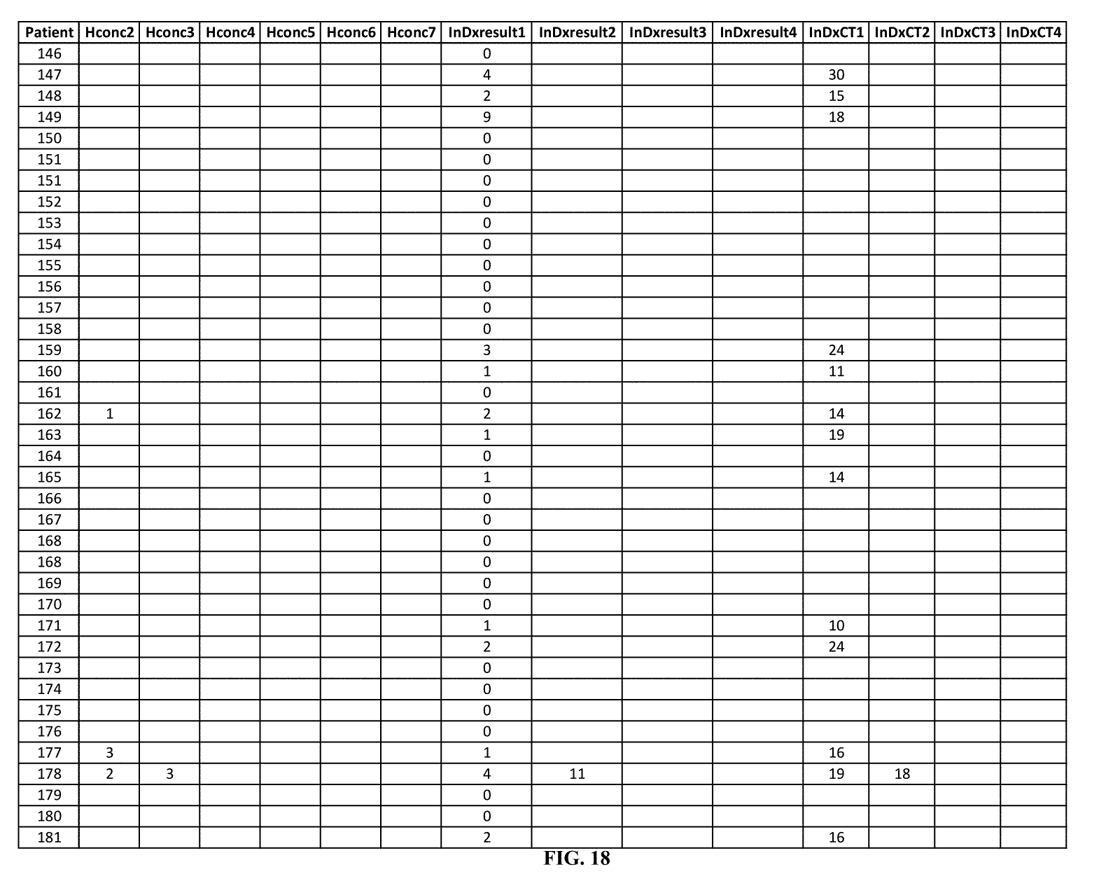

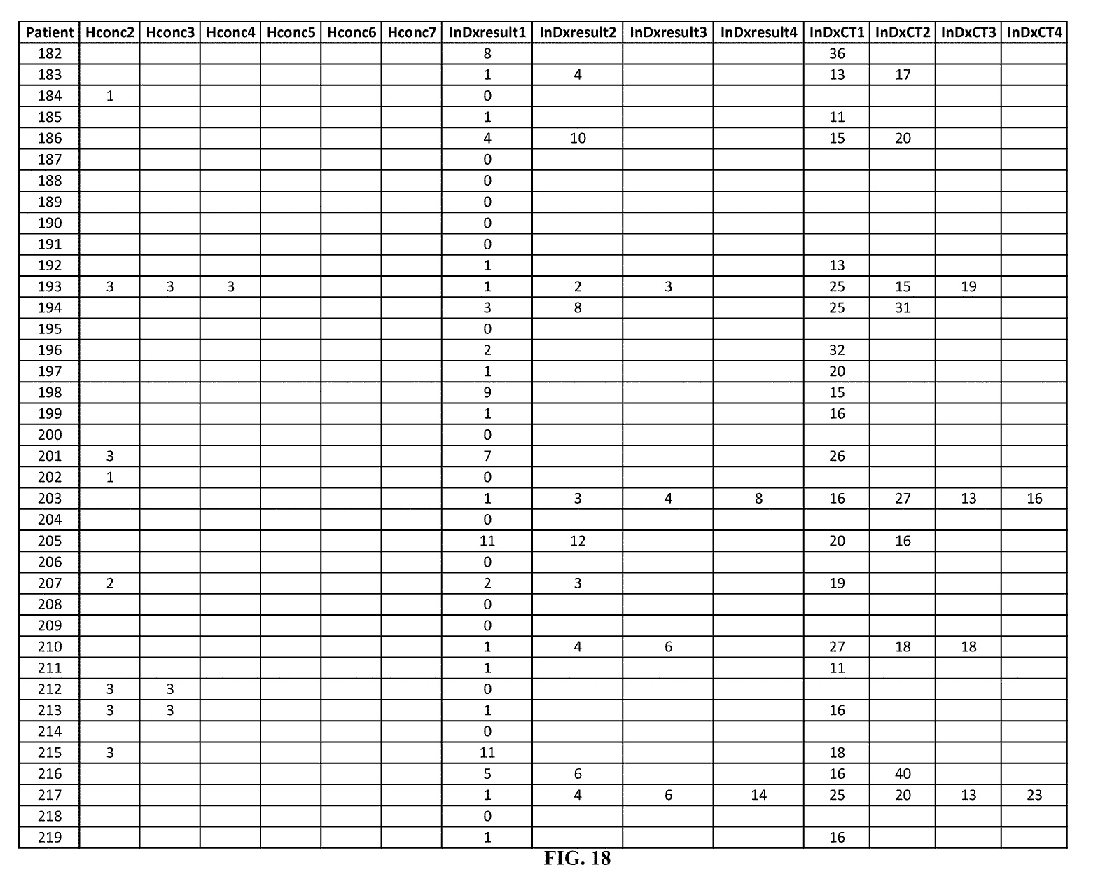

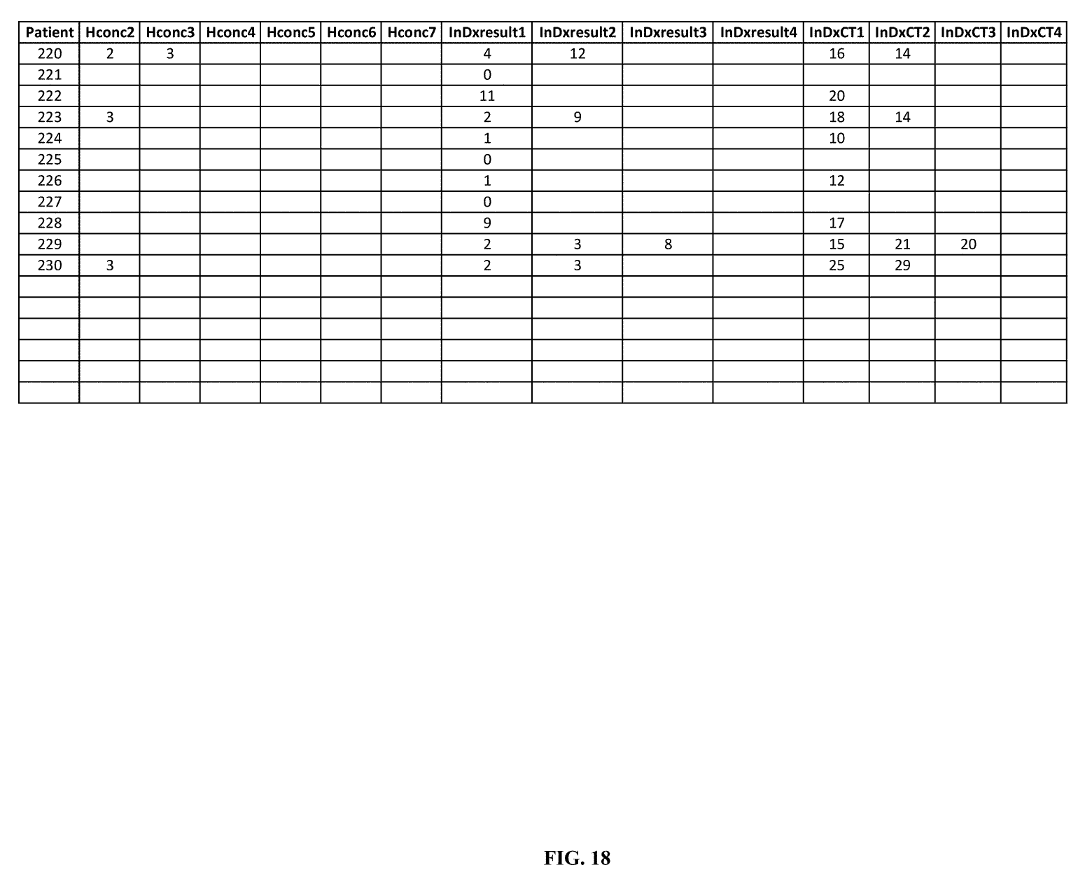

In one embodiment, the color change detection by EBT-LAMP was tested as a secondary direct amplification testing technique for 12 clinical samples included in the present disclosure (8 urine samples, 3 mucocutaneous swabs and 1 stool sample tested for C. difficile). In another embodiment, eleven of these were confirmed positives by the hospital testing including the stool sample that tested positive for the C. difficile toxin by the hospital testing. In addition, none of the samples were positive for mecA by the hospital testing or the EBT-LAMP analysis (as shown in FIG. 18).

### Method for Preparing a Shelf Stable Target Primer Example:

In various embodiments, the data related to the method for improving shelf stable target primers applies to the method 300, 400, 500 and 600.

In another embodiment, if the shelf stable target DNA/RNA primer is for the methods 300 and 500, the components include 86.4 uL of 2M trehalose (Sigma), 84.48 uL Bst 2.0 polymerase (New England Biosciences), 105.6 uL primer mix (Integrated DNA Technologies), 6 uL 50% glycerol (Thermo Fisher Scientific), 42.24 uL Pluronic F-68 (Gibco), 105.6 uL 10% Bovine Serum Albumin (New England Biosciences) and 59.04 uL 25 mM dNTP (Thermo Fisher Scientific), and 63.36 uL 100 mM MgSO4 (New England Biosciences) were added to a 1.5 ml Eppendorf tube. In various embodiments, the indicator is Eriochrome Black T dye or Syto82 dye. In one embodiment, 26.4 uL of 3 mM EBT (in ethanol) (Sigma-Aldrich) was added to the tube including the above listed components. Then the components were vortexed for 10 seconds and 5.48 uL of the above mixture including EBT was delivered to each well of the 96-well plate or PCR reaction tube.

In another embodiment, if the shelf stable target DNA/RNA primer is for the methods 400 and 500, the components include 86.4 μL of 2M trehalose, 84.48 μL Bst2.0 polymerase, 105.6 μL primer mix, 6 μL 50% glycerol, 42.24 μL pluronic F-68, 105.6 μL 10% BSA and 59.04 μL 25 mM dNTP, and 63.36 μL 100 mM MgSO4 were added to a 1.5 mL Eppendorf tube. In another embodiment, 42.24 μL of 500 μM Syto82 (in DMSO) (Thermo Fisher Scientific) was added to the tube including the above listed components. Then 5.63 μL of the above mixture including Syto82 was delivered to each well of 96-well plate or PCR reaction tube (Syto-82—method 400).

After the components and the mixture (EBT and Syto-82) are delivered to each well, the well is exposed to room temperature ±5° C. for 24 hours. By exposing the wells at room temperature (±5° C.) for 24 hours, the contents in the tube are able to dry.

The vials, wells, or the reaction tubes were sealed for future use of the detection of bacterial and fungal pathogens at clinically relevant concentrations in a clinical fluid sample.

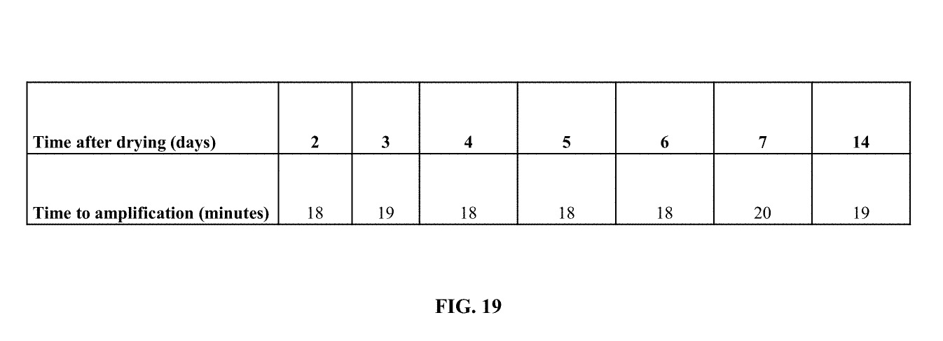

In another embodiment, a shelf-stable method for the methods 300, 400, and 500 assays reaction mixtures were tested for both Syto82 and EBT plates. Analysis of the plates at w, 3, 4, 5, 6, 7 and 14 days post-drying shows that amplification of positive controls is relatively unchanged at these intervals (as shown in FIG. 19). Additionally, the plate stability from room temperature stable Syto82 assay plate preparation was measured at daily intervals for one week and then on week two to assess the time to amplification over time (as shown in FIG. 19). In one embodiment, the time to amplification in minutes does not change significantly within a two week period. The studies are continuing at two week intervals to determine potential degradation over time.

In another embodiment, modification of reactions to a mixture that is stabilized with 2M trehalose obviates the need for fresh preparation of materials for each test. This allows printing of plates in batches with at least a two week room temperature shelf life without delay or degradation of positive signal. This will allow for large scale printing and transport of materials and faster preparation of reactions.

### Sensitivity, Specificity, Positive Predictive Value (PPV) and Negative Predictive Value (NPV)

In various embodiments, the analysis of 224 duplicate clinical blood, urine, wound/swab, sputum, stool, and cerebrospinal fluid samples taken from 248 prospectively-enrolled patients with suspected infection by the method 300, 400. And 500 has shown overall 78% sensitivity and 98% specificity compared to the gold-standard hospital culture and sensitivity testing for detection of the targeted clinical pathogens. The method 300, 400. And 500 across culture types shows a positive predictive value (PPV) of 61% and negative predictive value (NPV) of 99% compared to hospital methods. The methods 300, 400, and 500 are complete in an average of 46 minutes start to finish, and polymicrobial infection detection is equivalent or better than hospital cultures.

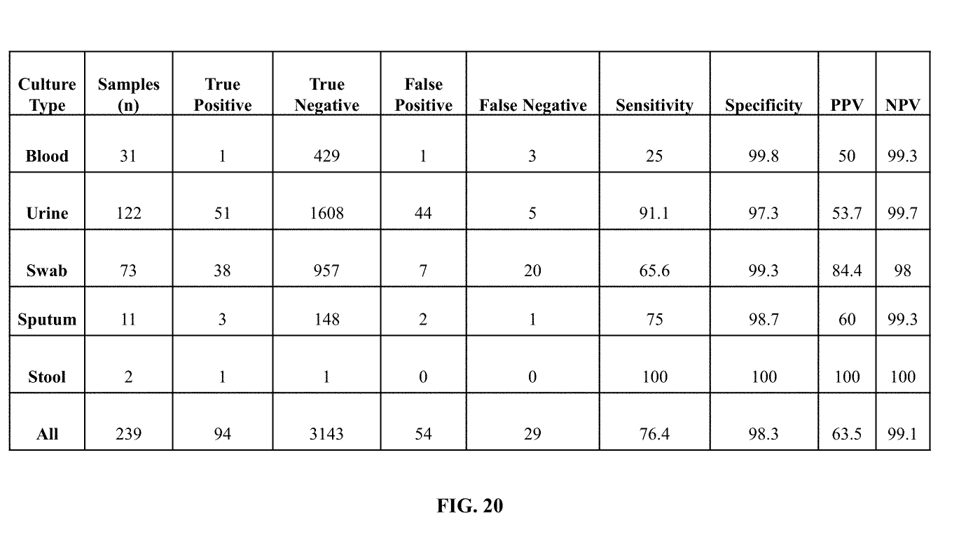

In another embodiment, a total of 239 samples were collected across culture types (31 blood, 122 urine, 73 mucocutaneous wound/swab, 11 sputum and two stool samples) from 229 consecutively enrolled patients with suspected clinical infection with samples analyzed both at the hospital and by the methods 300, 400, and 500. In one embodiment, nine patients had two sample types analyzed by the methods 300, 400, and 500. In another embodiment, the overall sensitivity of the methods 300, 400, and 500 for the detection of the target pathogens in the blood, urine, wound/swab, sputum, and stool samples from 239 clinical samples was 76.4% with a specificity of 98.3%. The positive predictive value for the samples was 63.5% and negative predictive value was 99.1%. (as shown in FIG. 20). In another embodiment, as illustrated in FIG. 20, the methods 300, 400, and 500 results are shown versus the hospital culture results by culture type. In addition, FIG. 20 illustrates a summary of data for performance of the methods 300, 400, and 500 across culture types for the 15 targets of the methods 300, 400, and 500. The samples show the total prospectively matched samples within each sample type. A true positive indicates an equivalent positive result in the hospital results and in the methods 300, 400, and 500 results. A true negative indicates equivalent negative results in both the hospital results and in the methods 300, 400, and 500 results. A false positive indicates negative results by the hospital culture and positive results by the methods 300, 400, and 500. A false negative indicates positive results by the hospital culture and negative results by the methods 300, 400, and 500. In one embodiment, the sensitivity, specificity, and positive and negative predictive values are expressed as percentages as described in the Statistical Analysis section (as illustrated in FIG. 17).

In various embodiments, the present disclosure includes methods (300, 400, and 500) that offer advantages in speed, sensitivity, specificity, scalability, flexibility in target selection, and conservation of resource utilization. Limitations revealed in the sample population studied include low sensitivity for bloodstream infection. This hindrance is due to a low number of samples studied, low positive rate among the samples studied, variability in the types of samples received (included were EDTA and Bactec aerobic culture bottles). Additionally, 3 mL of blood was processed to test against the 15 targets. Processing a larger volume of blood for the methods 300, 400, and 500 aids in clearing the threshold for pathogen detection directly from these samples for potential point-of-care blood testing. Alternative methodologies for target extraction from bloodstream including nanoparticles and microbeads and premixing reagents with stabilization of reactions at room temperature will help in ease of processing to increase potential for bedside diagnostics by clinical staff with limited training.

Clinical data corresponding to duplicate samples obtained for LAMP analysis were abstracted by retrospective chart review. Patient characteristics and laboratory values, including hospital culture results by organism isolated across culture types, were analyzed. The clinical samples with hospital culture or equivalent testing and with a corresponding duplicate sample for LAMP testing were included in the analysis (as shown in FIG. 17).

Standard measurements for statistical analysis were used to calculate:

Sensitivity (SE) or True Positive Rate (TPR)

SE=True Positive (TP)/(True Positive (TP)+False Negative (FN))

Specificity (SP) or true negative rate (TNR)=N targets−TP−False Positive (FP)−FN

SP=True Negative/(TN+FP)

Precision or Positive Predictive Value (PPV)

PPV=TP/(TP+FP)

Negative Predictive Value (NPV)

NPV=TN/(TN+FN)

Sensitivity for each organism was determined based on positive findings in the methods 300, 400, and 500 in comparison versus hospital methods abstracted from clinical results.

The present disclosure results provide evidence that direct amplification methodologies overcome many limitations of detection of infectious pathogens. The present disclosure has shown that low but clinically-relevant pathogen loads do not appear to limit detection of pathogens directly from urine, wound, sputum and stool samples. Relatively high sensitivities were found for the pathogens targeted by the methods 300, 400, and 500, especially with urine samples for which 3 mL of the clinical urine sample was used per panel run.

### Hospital Culture Analysis

Culture identification was performed using Siemens Microscan (Beckman Coulter, Inc.), BD Phoenix Automated Microbiology System (Becton, Dickinson and Company), or biochemical tests. Prior to revival of clinical samples for primer validation, isolates were stored in 15% glycerol stocks at −80° C. Isolates were revived by growing on tryptic soy broth (TSB) media overnight at 37° C. (no agitation) and serial diluted in 1× Phosphate Buffered Saline (PBS). Ten microliters of each serial dilution was plated on trypticase soy agar (TSA) plates (in triplicate) and colony forming units were counted following 24 h of incubation at 37° C.

In various embodiments, the microbial culture samples used for primer validation studies included Methicillin-resistant S. aureus, S. aureus, Streptococcus agalactiae, Streptococcus pyogenes, Enterococcus faecalis, Enterococcus faecium, Escherichia coli, Methicillin-resistant S. epidermidis, Proteus mirabilis, Klebsiella pneumoniae, C. difficile and Candida albicans. Each was a validated clinical culture sample from Sparrow Hospital. Enterococcus casseliflavus (ATCC: 25788), Enterococcus gallinarum (ATCC: 49673), and Pseudomonas aeruginosa (ATCC: 10145) are from American Type Culture Collection (ATCC, Manassas, Va., USA).

### Urine Samples

In one embodiment, 99 clinical urine samples tested by the methods 300, 400, and 500 showed a sensitivity of 91% overall for 14 targets compared to the standard hospital culture. The specificity was calculated to be 96%. The positive predictive value (PPV) for the presence of the 14 targets was 50% and the negative predictive value (NPV) was 99%. The time to completion of the positive urine culture testing was 2,477 minutes for hospitals versus 45 minutes for the methods 300, 400, and 500. Moreover, the methods 300, 400, and 500 detects polymicrobial infections better than hospital cultures.

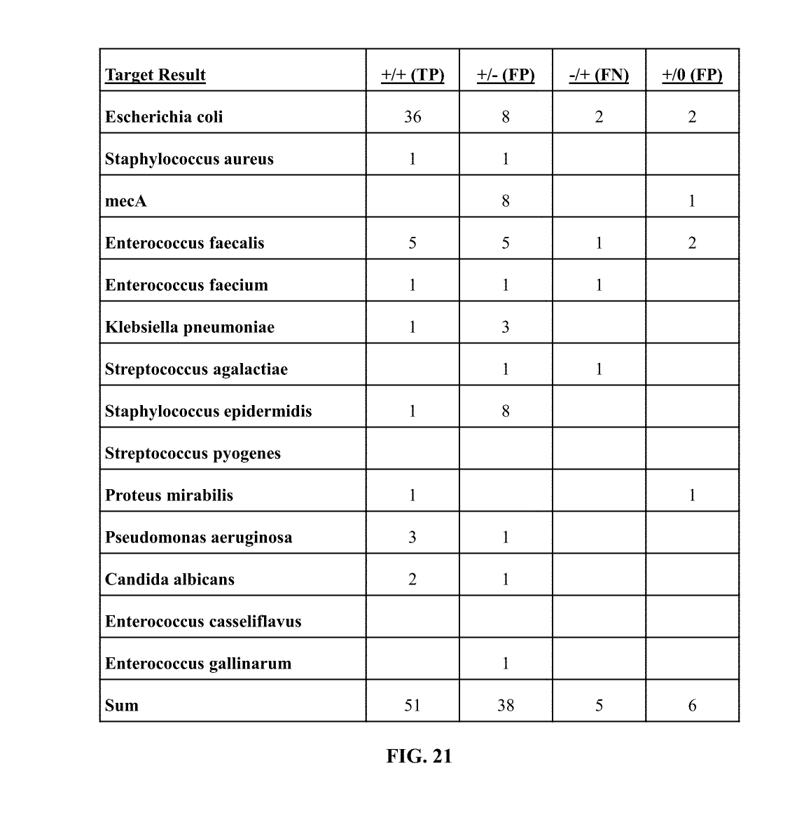

In one embodiment, the urine samples tested included 122 duplicate clinical samples that were tested for the presence of the 14 molecular targets, and 51 targets were equivalently detected by the hospital and the methods 300, 400, and 500 across these matching culture samples. In another embodiment, negative agreement was present for 1608 tests (including mecA identification) (e.g. shown in FIG. 20). The overall sensitivity of the methods 300, 400, and 500 for urine pathogen detection was 91.1% and specificity was 97.3%, with a positive predictive value of 53.7% and a negative predictive value of 99.7%. The methods 300, 400, and 500 identified six target pathogens corresponding to hospital culture samples that resulted as “normal flora” (as shown in FIG. 21). The method 300, 400, and 500 detected an additional 38 pathogen targets from clinical urine samples that were not positive by the hospital cultures. In another embodiment, eight of the pathogens found were S. epidermidis (Cₜ range 16 to 35) (as shown in FIG. 17), and four were associated with a positive mecA amplification. Another eight of the additional positive results by the method 300, 400, and 500 tested for were E. coli (Cₜ range 10 to 28). E. coli was the most common uropathogen detected by both the hospital culture (n=36) and the methods 300, 400, and 500 methods (n=48). The overall sensitivity for E. coli by the methods 300, 400, and 500 was 95% and specificity was 90%. In addition, the E. coli identification by the methods 300, 400, and 500 were positive for an alternate pathogen by the hospital culture results not targeted by the methods 300, 400, and 500 four times (Citrobacter koseri, Enterobacter cloacae, Candida species (non-albicans non-gabralta), and Klebsiella ornitholytica one time each). The methods 300, 400, and 500 did not identify a pathogen by direct amplification that was found by hospital urine culture five times: twice for E. coli, and once each for E. faecalis, E. faecium and S. agalactiae. Each of the tests were confirmed positive by the methods 300, 400, and 500 from cultures grown from residual reserved clinical sample. The cycles to threshold positive ranged from 9 to 28 minutes for samples with concordant positive results and >100,000 CFU/mL reported concentration (as shown in FIG. 18).

In another embodiment, as shown in FIG. 21, the Urinalysis comparison of the hospital culture results versus the thermocycler analysis provides Column 1, +In-Dx/+HCR 1-14, indicates true positive (FP) equivalent identification by both the methods 300, 400, and 500 and by hospital culture results for the 13 urinary tract infection species targets as well as mecA. Column 2, +In-Dx/−HCR 1-14, indicates false positive (FP) clinical samples identified as positive by the methods 300, 400, and 500 and negative by hospital culture result. Column 3 indicates false negative (FN) with negative result by the methods 300, 400, and 500 and positive by hospital culture; and Column 4 shows false positive (FP) samples found positive by the methods 300, 400, and 500 and called “Normal Flora” by hospital.

### Throat, Open Wound, and Abscess Samples

In various embodiments, duplicate throat, open wound, and abscess incision and drainage clinical samples (n=80) were analyzed between the hospital and the methods 300, 400, and 500. The panel shows that the sensitivity was 70% and the specificity 99% for the presence of the 14 target pathogens versus the hospital methods. Overall, the methods 300, 400, and 500 showed a PPV of 86% and NPV of 98% for the presence or the absence of the target organisms compared to the hospital wound/swab culture results. The time to results averaged 3448 minutes for hospitals and 49 minutes for the methods 300, 400, and 500.

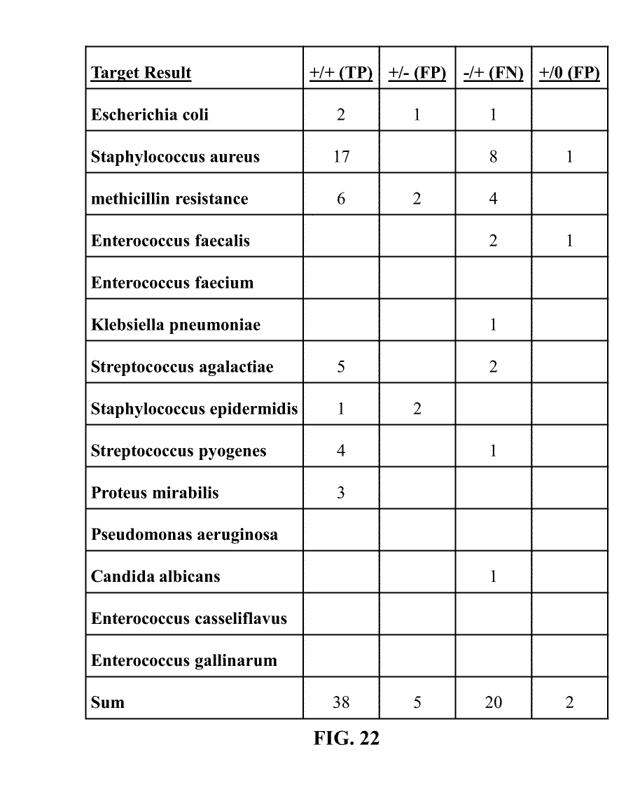

In various embodiments, 73 duplicate wound and throat swab samples were analyzed. The sensitivity for the presence of targets was 65.5% and specificity was 99.3% (e.g. shown in FIG. 11). In one embodiment, S. aureus was the most frequently detected pathogen among wound and swab samples (n=25 by hospital culture) and sensitivity for S. aureus detection by the methods 300, 400, and 500 was 68% with a specificity of 98%. Presence of mecA was found in association with detection of S. aureus and S. epidermidis as well as untargeted Citrobacter koseri (n=1) and unidentified background flora. The targets missed by the methods 300, 400, and 500 for the mucocutaneous swab analysis included S. aureus (n=8), mecA (n=4), E. faecalis (n=2) and S. agalactiae (n=2), E. coli, K pneumoniae, S. pyogenes and C. albicans (n=1 each) (as shown in FIG. 21). In another embodiment, the methods 300, 400, and 500 detected methicillin-resistant S. epidermidis in two samples, which were not identified by the hospital culture methods. The hospital methods identified three samples as “normal flora” but positive by the methods 300, 400, and 500 for S. aureus and E. faecalis once each (as shown in FIG. 22).

As shown in FIG. 22, the mucocutaneous swab comparison includes the hospital culture results versus the methods 300, 400, and 500 thermocycler analysis across targets: Column 1, +In-Dx=+HCR 1 to 14, indicates true positive (TP) equivalent identification by both the methods 300, 400, and 500 and by the hospital culture results for the 13 urinary tract infection species targets as well as mecA by target. Column 2, +In-Dx/−HCR 1-14, indicates a false positive (FP) clinical samples with positive results by the methods 300, 400, and 500 and negative for the hospital culture result. Column 3 indicates false negative (FN) results for the methods 300, 400, and 500 with positive results for the hospital culture and negative by the methods 300, 400, and 500; and Column 4 shows false positive (FP) samples with positive results for the methods 300, 400, and 500 and identification as “Normal Flora” for the hospital methods.

In various embodiments, numerous reasons account for under-detection of wound swabs. In most wound swab samples collected the duplicate second, third or even fourth swab was sent for analysis by the methods 300, 400, and 500. The clinical sample analysis preference was always given toward the hospital testing to avoid potential underdiagnoses of patient infection. Often, the duplicate sample for the methods 300, 400, and 500 analysis had a very small amount of clinical sample to be analyzed as a result of decreased sample availability following replicate collections.

In another embodiment, the amount of clinical swab saturation with sample appears to be directly correlated with direct amplification findings for positive samples. Although very little sample is needed, limits are certainly present for detection. The hospital clinical S. aureus and MRSA sample positives missed by the first pass using the methods 300, 400, and 500 and were retested on cultures grown in trypticase soy broth and were found to confirm the hospital culture findings. The clinical samples were grown in culture and those with positive growth in trypticase soy broth were retained and frozen for retesting. It is also possible that the lower sensitivity of detection from mucocutaneous swab samples is due to interfering substances present with the clinical sample. To determine the influence of interfering factors on reactions an internal control PCR set is included in future studies.

### Blood Samples

In another embodiment, out of 31 matched blood cultures analyzed, the hospital culture methods detected E. coli twice where the methods 300, 400, and 500 detected E. coli in one of the two samples. In another embodiment, the hospital detected MRSA once where the methods 300, 400, and 500 did not detect MRSA. In addition, the hospital detected one of two clinical blood culture samples taken from the same patient was positive for methicillin sensitive S. epidermidis and the methods 300, 400, and 500 was negative for S. epidermidis. The E. coli was detected from one sample by the methods 300, 400, and 500 for which one of two hospital blood cultures came back positive for unnamed micrococcus species. The sensitivity for the positive detection of the 14 targets from blood using the filtration methodology was 25% (n=¼). The negative predictive value was 99.3% as three positive cultures were called negative by the methods 300, 400, and 500 used. It is very likely that the micrococcus and S. epidermidis positive cultures, both positive for one of two blood collection tubes, are contaminants of little clinical value (as shown in FIG. 17).

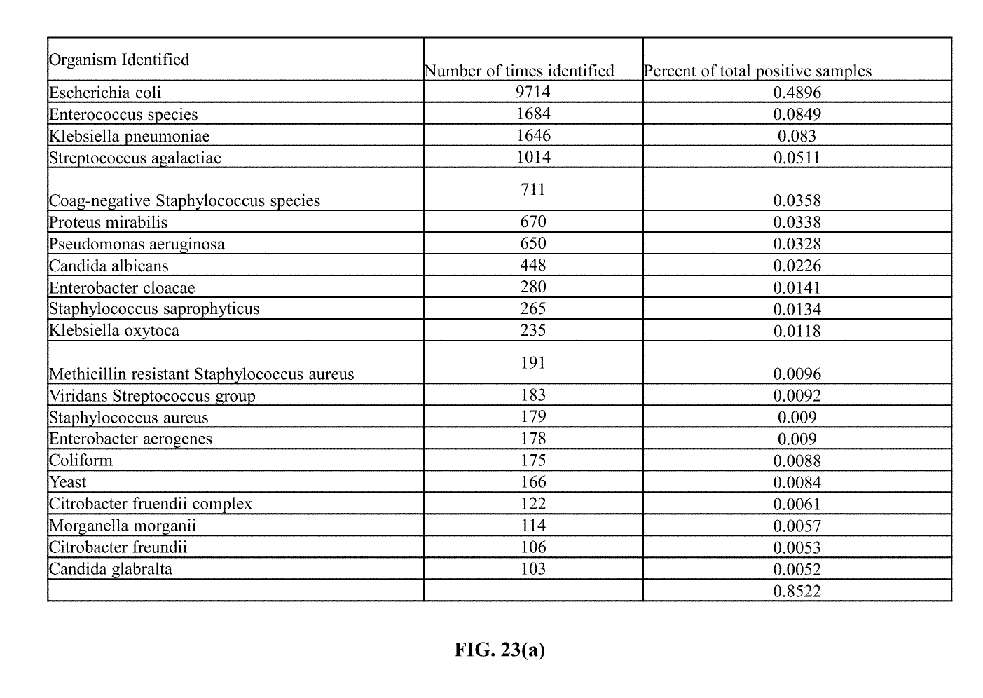

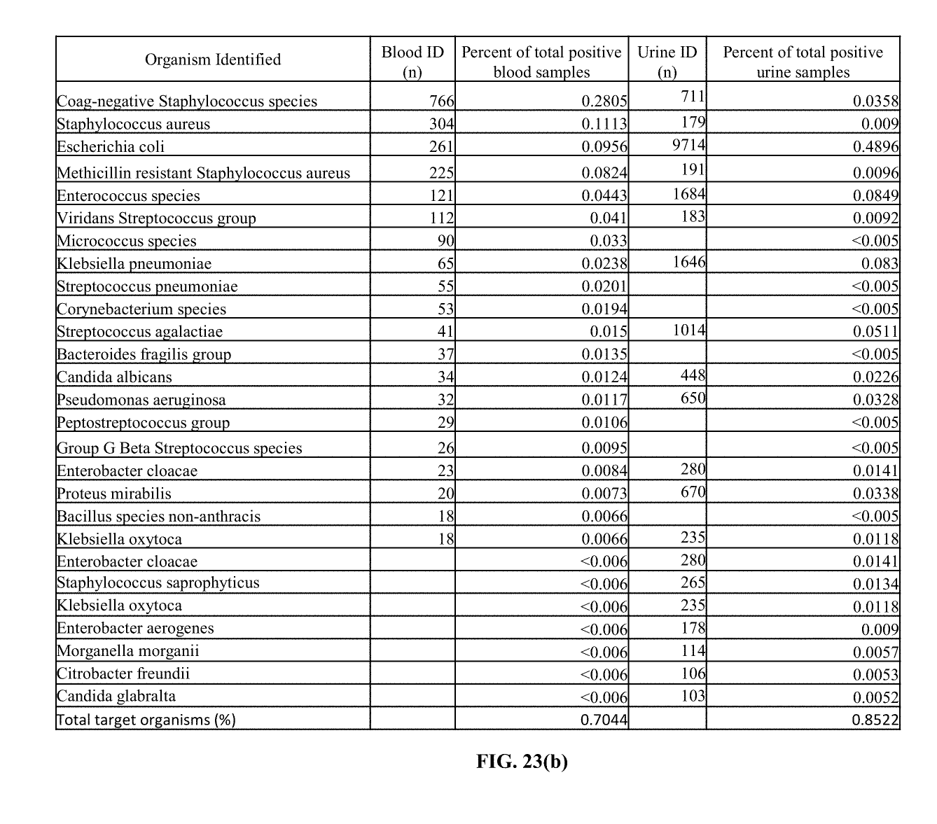

The present methods are robust yet sensitive to pathogen concentrations from samples across a diverse set of tissue types. The organisms targeted by the methods 300, 400, and 500 account for >70% of positive clinical blood and >85% of positive urine culture results in 2013 (as shown in FIG. 23(a)). The most sufficient clinical sample (>5 mL) is available from most urinalysis specimens for a great deal of molecular testing. Positive identification is clear from most samples after 20 minutes by isothermal amplification. In one embodiment, strongly positive clinical samples with >100,000 CFU/mL by culture averaged a Ct value of 14.4 min by LightCycler analysis (n=39). It does not seem likely by these results that false positive of clinical pathogens by the methods 300, 400, and 500 is a problem. The 91% overall sensitivity of the methods 300, 400, and 500 was higher for urine samples when compared to culture results from two different hospital institutions. In another embodiment, the false negative detection by the methods 300, 400, and 500 was low for the targeted organisms as compared to the hospital culture (5/121) (as shown in FIG. 18). The reasons for failure of these samples are unknown but one (Pt ID 222) failure was due to a reaction error as subsequent repeat testing from the culture was positive for E. faecalis as indicated by the hospital culture. The cultures did not show growth for other false negative samples.

In another embodiment, a low sensitivity (25%) was present for blood samples. The clinical sample size is too low to make confident predictions about utility of the process used here for direct amplification. Many of the clinical samples were collected from patients with stable vital signs and of a mild to moderate state of illness. In one embodiment, in-vitro testing suggests that Purple Top EDTA Blood Collection tubes are best for analysis using the methods 300, 400, and 500. A low abundance of target template is expected from the small amount of blood sampled for direct amplification analysis. LAMP methods have been used successfully to identify pathogens from blood that is already pre-cultured and shows signs of colony growth. The threshold for identification with alternate processing methods to enable detection of bacteremia is lowered.

In one embodiment, more critically ill patients would reasonably be expected to carry a higher concentration of pathogens per mL of circulating blood. The median Glasgow Coma Score among patients was 15 (out of 15) with an average shock index (heart rate/systolic blood pressure) of 0.71, an average mean arterial pressure (diastolic blood pressure+⅓ (systolic blood pressure−diastolic blood pressure)) of 99 and an average lactic acid concentration of 1.68 mg/dl. Therefore, a low percentage of these patients would be likely to have significant bacteremia. A larger sample size from a cohort of more critically septic patients will likely improve results of direct blood testing by the methods 300, 400, and 500.

### Sputum Samples

In another embodiment, eleven sputum samples were analyzed using the methods 300, 400, and 500. The pathogenic concentrations of E. coli (n=1), P. aeruginosa (n=1) and MRSA (n=3) were detected by the panel. In one embodiment, samples, agreement was reached for MRSA (n=3) and E. coli (n=1). The methods 300, 400, and 500 identified P. aeruginosa and E. coli one time each where the hospital culture was called negative and normal flora, respectively. In addition, the methods 300, 400, and 500 did not detect S. aureus in one sputum sample identified as positive by the hospital sputum culture methods. The Negative agreement was reached for nine samples (as shown in FIG. 18).

In one embodiment, two stool samples were studied using direct amplification methods for detection of C. difficile (as shown in FIG. 18). The C. difficile toxin was identified by both direct amplification methods and hospital C. difficile toxin screening methods for one of these samples. In addition, gastrointestinal pathogens are added to help guide clinical decision making for infectious diarrheal complaints.

In another embodiment, the sensitivity threshold and range displayed by the direct amplification methods (300, 400, and 500) appears to correlate much more strongly with “moderate” or “many” laboratory reported results for wound and sputum samples, and to >50,000 CFU/mL for urine output results. The outputs from clinical urine samples from Sparrow Hospital are presented in concentration ranges from 10,000-25,000, 25,000-50,000, 50,000-100,000 and >100,000 CFU/mL. E. coli was not detected twice from urine (once at a concentration of 10,000-25,000 CFU/mL, once >100,000 CFU/mL. 10,000-25,000 CFU/mL may be below the limit of detection for the direct amplification method for urine, at least for this probe set, though the 21 additional pathogens identified by the methods 300, 400, and 500 testing and substantially confirmed in culture argue toward potential under-detection by hospital clinical methods.

In various embodiments, results from direct amplification testing of sputum samples show great promise for LAMP methods to rapidly reveal potential infectious respiratory pathogens including P. aeruginosa. A panel with inclusion of more common respiratory pathogens will provide a valuable infection diagnostic tool. Moreover, more samples tested would fully address detection capabilities for sputum samples.

In another embodiment, S. aureus and MRSA were the most under-detected clinical targets. These pathogens are of the highest clinical abundance and were missed on the first pass POC testing about 30% of the time. The lactate dehydrogenase was chosen due to its relative low homology to the other known genetic sequences explored in silico and presumptive high abundance of mRNA and DNA for amplification given its core metabolic genetic function. It is noted that mecA detection lags approximately 4 min in each case likely reflecting lower marker abundance relative to LDH. This apparent decreased sensitivity translates to further under-detection of mecA by EBT color changed analysis from samples found to be positive by LightCycler analysis. The present disclosure uses an adjustment to a longer incubation to determine whether it will accommodate detection of lower-abundance molecular targets.

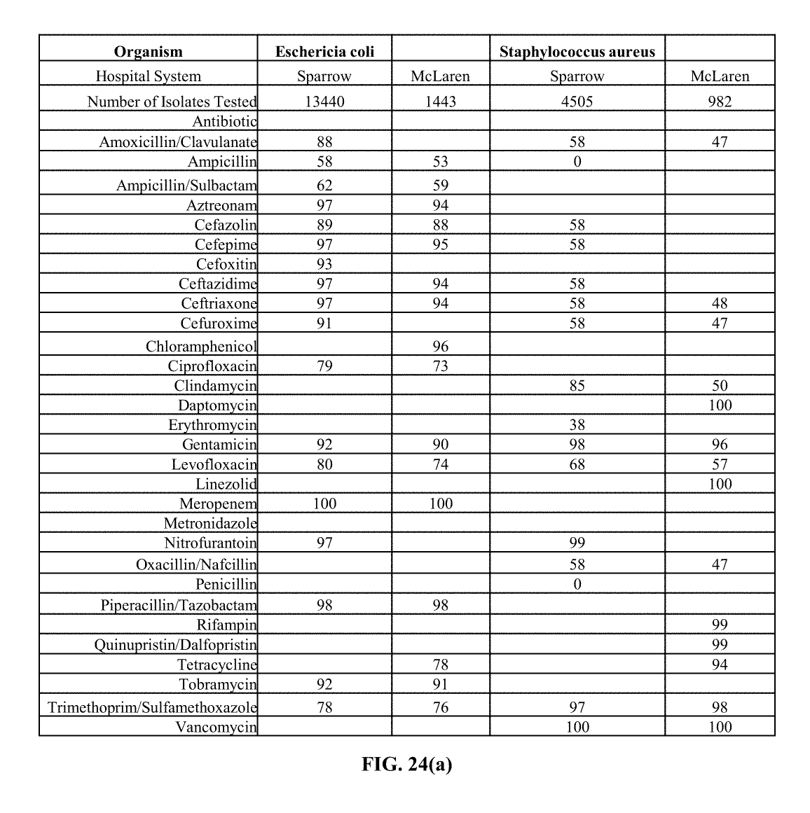

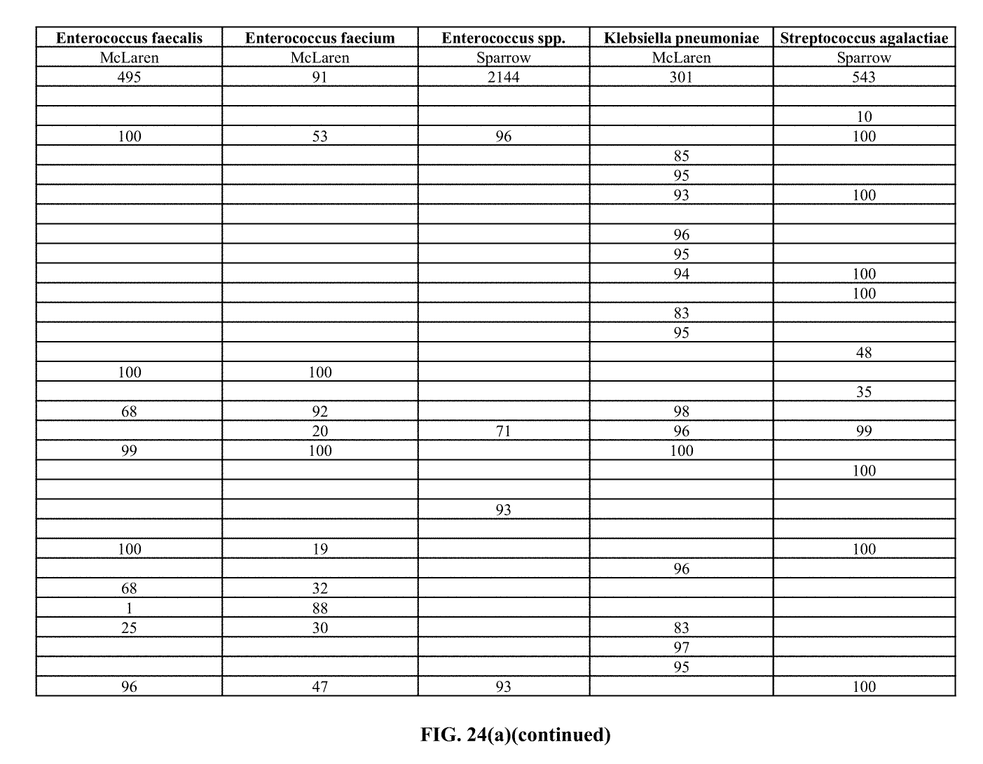

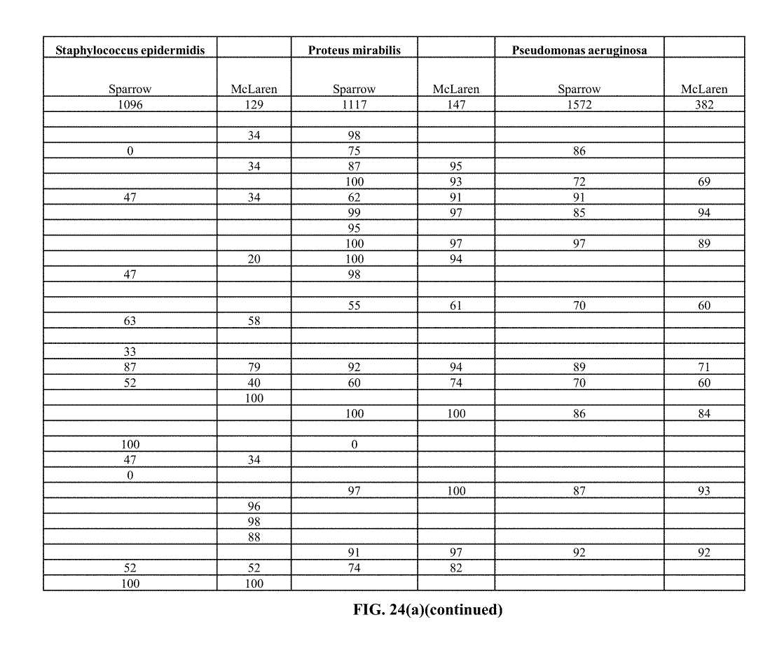

In various embodiments, the advantages to the rapid diagnostic methods (300, 400, and 500) employed in the present disclosure include amelioration of appropriate antimicrobial strategy selections including selection of antibiotics with a high degree of specificity for eradication of infection. For example, E. coli was 97% sensitive to nitrofurantoin and 89-97% sensitive to cephalosporin antibiotics versus lower sensitivities for more commonly prescribed empiric urinary tract infection antimicrobials such as trimethoprim/sulfamethoxazole (78%) and fluoroquinolones (79-80%) (as illustrated in FIG. 24: Sparrow and McLaren of Greater Lansing Hospital Antibiotic Sensitivity Summary 2015). In one embodiment, monomicrobial pathogenic infections by E. coli accounted for 48.96% of the urinary tract infections (n=9714) and 9.56% (n=261) of bloodstream infections in through the Sparrow Hospital clinical laboratory testing (as illustrated in FIG. 23(b): Sparrow Hospital Blood and Urine Culture Summary 2013). Moreover, similar precision with antibiotic prescription selection would be much easier to enact based on the results seen with direct amplification. As the burden of antibiotic resistance transmission increases, particularly within strains of enterobacteriaceae, precise antibiotic prescription is of increasing importance in antimicrobial therapy decision making.

In another embodiment, polymicrobial detection from clinically infected sources is equal to or greater to that of protracted culture methods. Positive polymicrobial cultures from clinical wound samples often revealed microbes of questionable clinical relevance including Corynebacterium species, micrococcus species, and peptostreptococcus species, all of which are likely nonpathogenic normal flora. General microscopic identification results with Gram stain and cell morphology but without genus and species designation assigned as final result outputs are often also presented without associated antibiotic sensitivity and resistance results, begging the question of clinical relevance, especially when reported concentrations are few, rare or moderate after four to six days of culture and modification of antibiotic therapy will continue to be empirical at best.

The results of the clinical samples tested indicate that the LAMP-based methods 300, 400, and 500 aid in the diagnosis of clinical infections for targeted pathogens across culture types. Further testing of additional samples across culture types and from additional institutions along with validation of additional organisms and antibiotic resistance genes for further expansion of the platform and characterization of the testing abilities and limitations is underway.

The ease of use, low cost, universality, and accuracy of the methods 300, 400, and 500 are utilized at the POC for improved patient treatment, outcomes and antibiotic stewardship. An immediate reward form accurate pathogen diagnostics is an inevitable upgrade in antibiotic prescriptive strategy and better patient outcomes, while helping to address the clear and present danger of broad-spectrum antimicrobial strategies threatening to further antibiotic resistance gene proliferation and undermine antimicrobial treatment efforts.

All combinations of the aforementioned embodiments throughout the entire disclosure are hereby expressly contemplated in one or more non-limiting embodiments even if such a disclosure is not described verbatim in a single paragraph or section above. In other words, an expressly contemplated embodiment may include any one or more elements described above selected and combined from any portion of the disclosure. In various non-limiting embodiments, all values and ranges of values between and including the aforementioned values are hereby expressly contemplated.

One or more of the values described above may vary by ±5%, ±10%, ±15%, ±20%, ±25%, etc. Unexpected results may be obtained from each member of a Markush group independent from all other members. Each member may be relied upon individually and or in combination and provides adequate support for specific embodiments within the scope of the appended claims. The subject matter of all combinations of independent and dependent claims, both singly and multiply dependent, is herein expressly contemplated. The disclosure is illustrative including words of description rather than of limitation. Many modifications and variations of the present disclosure are possible in light of the above teachings, and the disclosure may be practiced otherwise than as specifically described herein.

It is also to be understood that any ranges and subranges relied upon in describing various embodiments of the present disclosure independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present disclosure, and such ranges and subranges may be further delineated into relevant halves, thirds, quarters, fifths, and so on. As just one example, a range “of from 0.1 to 0.9” may be further delineated into a lower third, i.e. from 0.1 to 0.3, a middle third, i.e. from 0.4 to 0.6, and an upper third, i.e. from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

1. A method for detecting bacterial and fungal pathogens at clinically relevant concentrations in a clinical fluid sample using a visual color change test, said method comprising the steps of:
preparing the clinical fluid sample for heat lysing with water;
heat lysing the clinical fluid sample to form a lysate that optionally comprises bacterial and/or fungal DNA and RNA forming a first mixture;
mixing the lysate with a target DNA primer and/or target RNA primer, Eriochrome Black T dye, a polymerase enzyme, and a chemical reaction buffer in a vial to form a reaction mixture;
incubating the reaction mixture;
amplifying the optional bacterial and/or fungal DNA and RNA and the target DNA primer and/or the target RNA primer in the reaction mixture using loop mediated isothermal amplification;
cooling the reaction mixture for a predetermined amount of time thereby stopping the amplification; and
identifying a color change in the reaction mixture which is indicative of the presence of bacterial or fungal pathogens in the clinical fluid sample at the clinically relevant concentrations.

2. The method of claim 1 wherein the clinical fluid sample is a clinical blood sample, a clinical urine sample, a clinical mucocutaneous swab/wound sample, a clinical sputum sample, a clinical stool sample and/or a clinical cerebrospinal culture sample.

3. The method of claim 1 wherein the bacterial and fungal pathogens are chosen from Escherichia coli, Staphylococcus aureus, Enterococcus faecalis, Enterococcus faecium, Streptococcus pyogenes, Pseudomonas aeruginosa, Klebsiella pneumonia, Proteus mirabilis, Staphylococcus epidermidis, Streptococcus agalactiae, Candida albicans, Enterococcus casseliflavus, Enterococcus gallinarum, and Clostridium difficile.

4. The method of claim 1 that is completed within 35 to 120 minutes.

5. The method of claim 1 wherein the step of amplifying the reaction mixture using loop mediated isothermal amplification is maintained at a temperature of from 55 to 67° C.

6. The method of claim 1 wherein the step of heat lysing occurs at a temperature of from 90° C. to 110° C. for 10 to 20 minutes.

7. The method of claim 1 wherein the step of preparing the clinical fluid sample includes filtrating the clinical fluid sample to rapidly concentrate bacteria in the clinical fluid sample.

8. The method of claim 1 wherein the reaction mixture amplification can be detected by standard spectrophotometer readings of from 300 to 475 nm wavelengths.

9. The method of claim 1 wherein the step of cooling the reaction mixture occurs by immersing the reaction mixture in an ice bath or by placing the reaction mixture in a refrigerator or a freezer.

10. A method for detecting bacterial and fungal pathogens at clinically relevant concentrations in a clinical fluid sample, said method comprising the steps of:
preparing the clinical fluid sample for heat lysing with water;
heat lysing the clinical fluid sample to form a lysate that optionally comprises bacterial and/or fungal DNA and RNA forming a first mixture;
mixing the lysate with a target DNA primer and/or a target RNA primer, Syto 82 dye, a polymerase enzyme, and a chemical reaction buffer in a vial to form a reaction mixture;
incubating the reaction mixture;
amplifying the optional bacterial and/or fungal DNA and RNA and the target DNA primer and/or the target RNA primer in the reaction mixture using loop mediated isothermal amplification; and
analyzing the reaction mixture by thermocycler to detect the presence of bacterial or fungal pathogens in the clinical fluid sample at the clinically relevant concentrations.

11. The method of claim 10 wherein the clinical fluid sample is a clinical blood sample, a clinical urine sample, a clinical mucocutaneous swab/wound sample, a clinical sputum sample, a clinical stool sample and/or a clinical cerebrospinal culture sample.

12. The method of claim 10 wherein the bacterial and fungal pathogens are chosen from Escherichia coli, Staphylococcus aureus, Enterococcus faecalis, Enterococcus faecium, Streptococcus pyogenes, Pseudomonas aeruginosa, Klebsiella pneumonia, Proteus mirabilis, Staphylococcus epidermidis, Streptococcus agalactiae, Candida albicans, Enterococcus casseliflavus, Enterococcus gallinarum, and Clostridium difficile.

13. The method of claim 10 that is completed within 35 to 120 minutes.

14. The method of claim 10 wherein the step of amplifying the reaction mixture using loop mediated isothermal amplification is maintained at a temperature of from 55 to 67° C.

15. The method of claim 10 wherein the step of heat lysing occurs at a temperature of from 90° C. to 110° C. for 10 to 20 minutes.

16. The method of claim 10 wherein the step of preparing the clinical fluid sample includes filtrating the clinical fluid sample to rapidly concentrate bacteria in the clinical fluid sample.

17. A method for detecting bacterial and fungal pathogens at clinically relevant concentrations in clinical fluid samples using a visual color change test and a fluorescence test, said method comprising the steps of:
preparing a first clinical fluid sample for heat lysing with water;
heat lysing the first clinical fluid sample to form a first lysate that optionally comprises bacterial and/or fungal DNA and/or RNA forming a first mixture;
mixing the first lysate with a target DNA primer and/or a target RNA primer, Eriochrome Black T dye, a polymerase enzyme, and a chemical reaction buffer in a vial to form a first reaction mixture;
incubating the first reaction mixture;
amplifying the optional bacterial and/or fungal DNA and/or RNA and the target DNA primer and/or the target RNA primer in the first reaction mixture using loop mediated isothermal amplification;
cooling the first reaction mixture for a predetermined amount of time thereby stopping the amplification;
identifying a color change in the first reaction mixture which is indicative of the presence of bacterial or fungal pathogens in the first clinical fluid sample at the clinically relevant concentrations;
preparing a second clinical fluid sample for heat lysing with water;
heat lysing the second clinical fluid sample to form a second lysate that optionally comprises bacterial and/or fungal DNA and/or RNA forming a second mixture;
mixing the second lysate with a second target DNA primer and/or a second target RNA primer, Syto 82 dye, a polymerase enzyme, and a chemical reaction buffer in a vial to form a second reaction mixture;
amplifying the optional bacterial and/or fungal DNA and/or RNA and the second target DNA primer and/or the second target RNA primer in the second reaction mixture using loop mediated isothermal amplification; and
analyzing the second reaction mixture by thermocycler to detect the presence of bacterial or fungal pathogens in the second clinical fluid sample at the clinically relevant concentrations.

18. The method of claim 17 wherein the predetermined clinical fluid samples each independently include a clinical blood sample, a clinical urine sample, a clinical mucocutaneous swab/wound sample, a clinical sputum sample, a clinical stool sample and/or a clinical cerebrospinal culture sample.

19. The method of claim 17 wherein the bacterial and fungal pathogens are chosen from Escherichia coli, Staphylococcus aureus, Enterococcus faecalis, Enterococcus faecium, Streptococcus pyogenes, Pseudomonas aeruginosa, Klebsiella pneumonia, Proteus mirabilis, Staphylococcus epidermidis, Streptococcus agalactiae, Candida albicans, Enterococcus casseliflavus, Enterococcus gallinarum, and Clostridium difficile.

20. The method of claim 17 wherein the step of amplifying is maintained at a temperature of from 55 to 67° C.

21. The method of claim 17 wherein the step of heat lysing occurs at a temperature of from 90° C. to 110° C. for 10 to 20 minutes.

22. The method of claim 17 wherein the reaction mixture amplification can be detected by standard spectrophotometer readings of from 300 to 475 nm wavelengths.

23. The method of claim 17 wherein the step of cooling the reaction mixture occurs by immersing the reaction mixture in an ice bath or by placing the reaction mixture in a refrigerator or a freezer.

24. A method for improving a shelf stable target DNA/RNA primer for the detection of bacterial and fungal pathogens at clinically relevant concentrations in a clinical fluid sample, said method comprising the steps of:
adding a plurality of components to a vial, wherein the components are chosen from a predetermined amount of: trehalose, polymerase, a primer mix, glycerol, a surfactant, a serum albumin, dNTP, and magnesium sulfate;
adding an indicator and a reaction buffer to the vial thereby forming a mixture;
vortexing the vial comprising the mixture for a predetermined amount of time;
exposing the mixture to room temperature ±5° C. for 24 hours; and
sealing the vial for future use of the detection of bacterial and fungal pathogens at clinically relevant concentrations in a clinical fluid sample.

25. The method of claim 24 wherein the indicator is Eriochrome Black T dye or Syto82 dye.


SEQUENCE LISTING (54 sequences)

SEQ ID NO: 1 - DNA, 19 bp, Escherichia coli
  primer_bind (1)..(19)
gagatatcgacccctcttg

SEQ ID NO: 2 - DNA, 22 bp, Escherichia coli
  primer_bind (1)..(22)
aatctgaaaaacggtagaaagt

SEQ ID NO: 3 - DNA, 42 bp, Escherichia coli
  primer_bind (1)..(42)
tccacagcaaaataactgcccaacatatatctcaggggacca

SEQ ID NO: 4 - DNA, 40 bp, Escherichia coli
  primer_bind (1)..(40)
gatgtctatcaggcgcgttttgccgtattaacgaacccgg

SEQ ID NO: 5 - DNA, 24 bp, Escherichia coli
  primer_bind (1)..(24)
tgtggttaataacagacaccgatg

SEQ ID NO: 6 - DNA, 24 bp, Escherichia coli
  primer_bind (1)..(24)
accatcttcgtctgattattgagc

SEQ ID NO: 7 - DNA, 18 bp, Escherichia coli
  primer_bind (1)..(18)
tatctaccgctcgcgtcg

SEQ ID NO: 8 - DNA, 18 bp, Escherichia coli
  primer_bind (1)..(18)
cgagcatctcttcagcgt

SEQ ID NO: 9 - DNA, 41 bp, Escherichia coli
  primer_bind (1)..(41)
tcctttgcccgaatcgcatcttagtgaaggcgaacagttcc

SEQ ID NO: 10 - DNA, 40 bp, Escherichia coli
  primer_bind (1)..(40)
tcgataacgtgctgatggtgcatgcgagtcggtagggttg

SEQ ID NO: 11 - DNA, 25 bp, Escherichia coli
  primer_bind (1)..(25)
cgtaaagtagaacggtttgtggtta

SEQ ID NO: 12 - DNA, 22 bp, Escherichia coli
  primer_bind (1)..(22)
cacgcataatggactggattgg

SEQ ID NO: 13 - DNA, 19 bp, Staphylococcus aureus
  primer_bind (1)..(19)
gatgctggtacaggtatyc

SEQ ID NO: 14 - DNA, 19 bp, Staphylococcus aureus
  primer_bind (1)..(19)
tttgcatgtgttgttacgt

SEQ ID NO: 15 - DNA, 46 bp, Staphylococcus aureus
  primer_bind (1)..(46)
gcrtttgtttctgatggcttattgagtgaatacaacgatggaacat

SEQ ID NO: 16 - DNA, 44 bp, Staphylococcus aureus
  primer_bind (1)..(44)
taacgacaaatcaagatggcacagcatttgttttgcttggtttg

SEQ ID NO: 17 - DNA, 23 bp, Staphylococcus aureus
  primer_bind (1)..(23)
tcttggtctcgcttcatatccaa

SEQ ID NO: 18 - DNA, 22 bp, Staphylococcus aureus
  primer_bind (1)..(22)
gtawcatatggcgctcgcccaa

SEQ ID NO: 19 - DNA, 23 bp, Staphylococcus aureus
  primer_bind (1)..(23)
aacagtatatagtgcaacttcaa

SEQ ID NO: 20 - DNA, 21 bp, Staphylococcus aureus
  primer_bind (1)..(21)
ctttgtcaaactcgacttcaa

SEQ ID NO: 21 - DNA, 47 bp, Staphylococcus aureus
  primer_bind (1)..(47)
atgtcattggttgacctttgtacataaattacataaagaacctgcga

SEQ ID NO: 22 - DNA, 48 bp, Staphylococcus aureus
  primer_bind (1)..(48)
tattggtkgatacacctgaaacaaaatttttttcgtaaatgcacttgc

SEQ ID NO: 23 - DNA, 25 bp, Staphylococcus aureus
  primer_bind (1)..(25)
atttaaccgtatcaccatcaatcgc

SEQ ID NO: 24 - DNA, 25 bp, Staphylococcus aureus
  primer_bind (1)..(25)
aggtgtagagaaatatggtcctgaa

SEQ ID NO: 25 - DNA, 21 bp, Staphylococcus aureus
  primer_bind (1)..(21)
atctcatatgctgttcctgta

SEQ ID NO: 26 - DNA, 21 bp, Staphylococcus aureus
  primer_bind (1)..(21)
aaaaaacgagtagatgctcaa

SEQ ID NO: 27 - DNA, 45 bp, Staphylococcus aureus
  primer_bind (1)..(45)
aatgcagaaagaccaaagcatacatgccaattccacattgtttcg

SEQ ID NO: 28 - DNA, 44 bp, Staphylococcus aureus
  primer_bind (1)..(44)
tgacgctatgatcccaatctaactactacggtaacattgatcgc

SEQ ID NO: 29 - DNA, 31 bp, Staphylococcus aureus
  primer_bind (1)..(31)
tttaaaatcagaacgtggtaaaattttagac

SEQ ID NO: 30 - DNA, 33 bp, Staphylococcus aureus
  primer_bind (1)..(33)
ccacataccatcttctttaacaaaattaaattg

SEQ ID NO: 31 - DNA, 25 bp, Streptococcus spp
  primer_bind (1)..(25)
tgtatagattgtagctctatcagtt

SEQ ID NO: 32 - DNA, 21 bp, Streptococcus spp
  primer_bind (1)..(21)
aagccttaacagatgtgattg

SEQ ID NO: 33 - DNA, 50 bp, Streptococcus spp
  primer_bind (1)..(50)
tccatttgcttcagttgattcaattcaggataagttaaaaccttttgttc

SEQ ID NO: 34 - DNA, 48 bp, Streptococcus spp
  primer_bind (1)..(48)
tgcgaataaccagcttagttatcccactttttcaactcaacatttagc

SEQ ID NO: 35 - DNA, 27 bp, Streptococcus spp
  primer_bind (1)..(27)
gctcaagttaacgatgtaaaggcatta

SEQ ID NO: 36 - DNA, 28 bp, Streptococcus spp
  primer_bind (1)..(28)
tcccatatcaatatttgcttgactaacc

SEQ ID NO: 37 - DNA, 25 bp, Streptococcus spp
  primer_bind (1)..(25)
gctgatgtgattttctataatggta

SEQ ID NO: 38 - DNA, 21 bp, Streptococcus spp
  primer_bind (1)..(21)
caatcaattgtttggcaatgt

SEQ ID NO: 39 - DNA, 45 bp, Streptococcus spp
  primer_bind (1)..(45)
acggcaaagtaatctttgtttttcgcaatctagaagatggtgggc

SEQ ID NO: 40 - DNA, 41 bp, Streptococcus spp
  primer_bind (1)..(41)
acttggaaggtgcaagcgaaaaatgattccgttttcgagat

SEQ ID NO: 41 - DNA, 25 bp, Streptococcus spp
  primer_bind (1)..(25)
gcatttttcactagtttggtgaacc

SEQ ID NO: 42 - DNA, 24 bp, Streptococcus spp
  primer_bind (1)..(24)
gaaaagaagatccacatgcttggt

SEQ ID NO: 43 - DNA, 18 bp, Streptococcus spp
  primer_bind (1)..(18)
cacacgtgtcagggatct

SEQ ID NO: 44 - DNA, 19 bp, Streptococcus spp
  primer_bind (1)..(19)
ccttagctcccaagttgac

SEQ ID NO: 45 - DNA, 38 bp, Streptococcus spp
  primer_bind (1)..(38)
tcaggcatcgcaccttctagtgtcaggaaatgctccat

SEQ ID NO: 46 - DNA, 42 bp, Streptococcus spp
  primer_bind (1)..(42)
tcaattgcttttgatgcgtgtctctctgatagcttgagcgta

SEQ ID NO: 47 - DNA, 23 bp, Streptococcus spp
  primer_bind (1)..(23)
gcggtaaggttctttcgtttcag

SEQ ID NO: 48 - DNA, 25 bp, Streptococcus spp
  primer_bind (1)..(25)
aatggactagcagactatgctcgta

SEQ ID NO: 49 - DNA, 18 bp, Streptococcus spp
  primer_bind (1)..(18)
cacaaggcattgaccctg

SEQ ID NO: 50 - DNA, 23 bp, Streptococcus spp
  primer_bind (1)..(23)
gcacctttttaaatttttgagtg

SEQ ID NO: 51 - DNA, 45 bp, Streptococcus spp
  primer_bind (1)..(45)
agctctttagcgatattaacagctttttatgacccacatacctgg

SEQ ID NO: 52 - DNA, 44 bp, Streptococcus spp
  primer_bind (1)..(44)
aggacgtttggatcctaaacacaatcttcagttagttgctctgc

SEQ ID NO: 53 - DNA, 20 bp, Streptococcus spp
  primer_bind (1)..(20)
ccagctaaaacgggatccgt

SEQ ID NO: 54 - DNA, 25 bp, Streptococcus spp
  primer_bind (1)..(25)
acagttacactaaaaaggctaaggc